SEQUENCE LISTING (144 sequences)

SEQ ID NO: 1 - DNA, 1647 bp, Lactococcus lactis
  misc_feature: kivD gene from Lactococcus lactis IFPL730
atgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataa

SEQ ID NO: 2 - DNA, 2433 bp, Artificial Sequence
  Synthetic: Tet-kivD construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataatacgcatggcatggatgaattgtataaataa

SEQ ID NO: 3 - DNA, 5739 bp, Artificial Sequence
  Synthetic: Tet-bkd construct sequence
gtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgtccgactacgagccactccgcttgcacgtgccggagccgacaggtcgtcccggctgcaaaacggatttctcttacctgcacttatctcccgcaggtgaagtccgcaaaccgcctgtcgacgtggagcctgcagaaaccagcgatttggcatattcgctggtgcgtgtgctcgatgatgatggacatgcagtgggtccgtggaatccgcagctctcaaacgaacagctgctgcgtggaatgcgcgcgatgctgaagacgcgtctgttcgatgctcgcatgttgactgcgcagcgccaaaaaaaattgagtttttatatgcagtgcttaggagaagaggcaatcgcgactgcccatacactggccctgcgcgatggtgatatgtgttttccgacgtaccgtcagcaggggattcttattacacgtgagtatccgcttgtggatatgatctgccagctgctgtcgaatgaagcggaccccctgaaaggccgtcaactgccgatcatgtacagcagtaaggaggctggcttctttagcatctcgggcaatcttgcgactcagtttattcaggcggtggggtgggggatggcaagcgcaatcaaaggggatacccgcattgcatccgcatggattggcgatggcgctaccgcggaaagcgattttcatacggcgctgacctttgctcacgtttatcgcgcaccggtgatcctcaatgtggtcaacaaccagtgggcgatttcgacgtttcaggccatcgcgggcggcgagggcaccacgttcgcgaaccgtggcgtgggttgcggcattgcgagcctccgtgtggacgggaacgattttttggccgtgtatgcggcgagcgaatgggcggcagaacgcgcacgccgtaacttgggaccgtccctgatcgaatgggtaacttatcgcgcgggcccacacagcacgagcgacgatccgtcaaagtatcgccctgcggatgattggaccaattttccgctgggtgacccgattgcgcgtctgaaacgtcacatgatcggtttgggtatttggagcgaagaacagcacgaagctacgcacaaagcgctggaagcggaagtcctggcggcgcagaagcaggccgaaagccatggcactctgattgacggccgtgtgccgtctgcagcctctatgttcgaagatgtttatgccgagttacccgagcacttacgtcgccagcgccaggagctcggggtatgaacgccatgaacccgcagcatgaaaacgcgcaaaccgtgacctccatgacgatgattcaggccctgcgctcggcgatggatattatgttagaacgtgacgatgacgtcgtggtgtttggtcaggacgtagggtattttgggggagtgtttcgttgtaccgaggggttgcaaaagaagtatggtacgagtcgcgtcttcgatgcaccgatcagcgaatcaggcattatcggcgctgccgtgggcatgggtgcatatggcttgcgccctgtggttgaaattcagtttgcagattatgtatatcccgcgtctgaccaactgattagtgaggcggcacgcctccgctaccgtagcgcgggcgatttcattgtcccgatgaccgtccgcatgccttgtggagggggcatttacggtggccaaacgcattctcagagtccagaagccatgttcacacaagtgtgcggtcttcgcaccgtgatgccatctaatccttatgacgccaaaggattactgattgcgtgcatcgaaaacgacgatccggttatctttttagaacccaaacgtctgtacaacggtcctttcgacggtcatcacgaccgtcctgtcacgccgtggagcaaacatccggcatcgcaagtcccggatgggtattataaagtgcctctggacaaagcagcgattgtccgccctggtgcagcccttacagtcctgacgtatggtaccatggtgtacgtggcgcaggccgcggcagatgaaaccggcctcgatgcggagattatcgacctccgcagtctgtggccgctggacttggaaactatcgtcgcgagtgtgaaaaagaccggtcgttgtgttattgcccatgaagcgactcgtacctgcggctttggcgccgaactgatgtccctggtgcaggaacactgttttcaccatcttgaggctccgattgaacgcgtcactggctgggacacaccgtaccctcatgcgcaggaatgggcctatttcccgggcccagcgcgcgtgggagccgcctttaaacgcgtgatggaggtctgaatgggtacccacgttattaaaatgcctgatattggtgaaggcatcgcggaggtagagctggttgaatggcacgttcaagtgggtgatagcgtgaatgaagatcaggtactcgcggaagtaatgacggacaaagcaacggttgaaatcccgtcccctgttgctggccgcatcttggcactgggtggccagccgggacaagttatggcggtgggaggagaattaattcgcctggaagtggagggtgccggaaacctggcggagtctccggccgcagctacgcccgccgctccggtggcagcaactccggaaaaacctaaagaagcaccggttgcagcgccaaaagcagctgccgaagcaccccgtgcgcttcgtgattctgaagcgccgcgccaacgccgccagccgggggaacgcccattagcatcaccggccgtccgtcagcgtgcccgcgacctgggaatcgagctgcagtttgttcagggctctggcccagccggccgcgtgcttcatgaggacctggatgcgtatcttacgcaggatggaagtgttgctcgttcaggcggcgctgcgcagggttacgcggaacgccatgatgaacaggcagtcccggtgatcggtctgcgccgcaaaattgcccagaagatgcaggatgctaaacgccgcattcctcacttcagttacgtcgaagagattgacgtaaccgatctggaagccctgcgcgctcacttgaatcagaaatggggcgggcaacgtggtaaactgacgctgctgcctttcctcgtccgcgcaatggtcgtcgcattacgcgatttcccgcaactgaatgctcgctatgatgatgaagcggaagtagtgacgcgttacggggccgttcatgttggtatcgcgacccagtcagataatgggctcatggttccggtgttgcgccatgcagaaagccgtgacctgtggggtaatgcgtcggaagttgcgcgtctggccgaagcggcgcgttccggtaaagcgcaacgtcaggaactgagcggctccacgattaccctgtcaagccttggtgtgttgggagggattgtatccacgccagtcattaatcacccggaagttgcaatcgttggtgttaaccgtattgtggagcgccctatggttgttggtggtaatattgtagtacgtaaaatgatgaatctgagctcttcgtttgatcatcgcgtggtggacggcatggatgctgcggcttttattcaagccgtgcgcggtttgttagaacatcctgccaccctgttcctggagtaagcgatgagtcagattttaaaaacctcgctcctgatcgttggcggcgggccaggcggctatgtggcggcgatccgcgccggccagctggggattccaacggtgttggttgagggcgccgctttgggcggtacttgcctgaatgtggggtgcattccgagcaaagcgttgatccatgctgccgaagagtaccttaaagcgcgccactatgcatcacgttccgcgctgggcatccaggtgcaagcaccttcaattgacatcgcccgcaccgtggaatggaaagacgccattgtggaccgtttgacttcgggtgtggcggctctgctgaaaaagcatggtgtggatgtagtacaaggatgggcacgcatcctcgacggcaagagcgtggcggttgaactggcgggcggggggtcgcagcgcatcgagtgtgaacatctgcttctggcggcgggctcacaaagcgttgaattacccatcctgcctctggggggcaaagtaatcagcagcaccgaagcattagctccggggtcgttgccaaaacgtctggtggttgtgggtggcggttatattggtctggagctgggcactgcatatcgcaagctgggtgttgaagttgctgtggtggaggcacaaccccgcatcctgccgggctacgatgaggaactgactaagccggtggcccaagcgctgcgccgtctgggtgtagaactgtacctgggtcattcattgctgggaccgagtgaaaacggcgttcgcgtgcgtgatggggctggcgaagaacgtgagatcgccgcggaccaggtccttgtcgcagttggccgcaaaccgcgttcagagggttggaacctggagtctctcggtttagacatgaatgggcgtgccgtaaaagtggacgatcagtgccgtacaagcatgcgtaacgtatgggccattggcgacctggcgggcgaaccgatgctggcgcaccgcgctatggcgcaaggagaaatggtcgccgaattgattgcgggcaaacgccgtcagtttgcgccggttgcaattcctgcagtctgttttacggatccggaagtggtggtggcgggtctgagtccggaacaggccaaagatgcgggtctggattgcctggtcgcgtcattcccgttcgcagccaacggccgcgccatgacgttggaagctaacgaaggctttgtccgcgtggtggcacgtcgtgacaaccatctggtggttggttggcaggcggtcggtaaagctgtgtctgaattaagcaccgcgttcgcacaatctctggaaatgggcgctcgcctcgaagacattgcaggcacaatccacgcgcaccccaccctgggtgaagctgttcaggaagcggcactccgtgccttaggtcacgccctgcacatttga

SEQ ID NO: 4 - DNA, 6781 bp, Artificial Sequence
  Synthetic: Tet-leuDH-bkd construct
gtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgttcgatatgatggatgcagcccgcctggaaggcctgcacctcgcccaggatccagcgacgggcctgaaagcgatcatcgcgatccattccactcgcctcggcccggccttaggcggctgtcgttacctcccatatccgaatgatgaagcggccatcggcgatgccattcgcctggcgcagggcatgtcctacaaagctgcacttgcgggtctggaacaaggtggtggcaaggcggtgatcattcgcccaccccacttggataatcgcggtgccttgtttgaagcgtttggacgctttattgaaagcctgggtggccgttatatcaccgccgttgactcaggaacaagtagtgccgatatggattgcatcgcccaacagacgcgccatgtgacttcaacgacacaagccggcgacccatctccacatacggctctgggcgtctttgccggcatccgcgcctccgcgcaggctcgcctggggtccgatgacctggaaggcctgcgtgtcgcggttcagggccttggccacgtaggttatgcgttagcggagcagctggcggcggtcggcgcagaactgctggtgtgcgacctggaccccggccgcgtccagttagcggtggagcaactgggggcgcacccactggcccctgaagcattgctctctactccgtgcgacattttagcgccttgtggcctgggcggcgtgctcaccagccagtcggtgtcacagttgcgctgcgcggccgttgcaggcgcagcgaacaatcaactggagcgcccggaagttgcagacgaactggaggcgcgcgggattttatatgcgcccgattacgtgattaactcgggaggactgatttatgtggcgctgaagcatcgcggtgctgatccgcatagcattaccgcccacctcgctcgcatccctgcacgcctgacggaaatctatgcgcatgcgcaggcggatcatcagtcgcctgcgcgcatcgccgatcgtctggcggagcgcattctgtacggcccgcaataatgaaggagatatacatatgtccgactacgagccactccgcttgcacgtgccggagccgacaggtcgtcccggctgcaaaacggatttctcttacctgcacttatctcccgcaggtgaagtccgcaaaccgcctgtcgacgtggagcctgcagaaaccagcgatttggcatattcgctggtgcgtgtgctcgatgatgatggacatgcagtgggtccgtggaatccgcagctctcaaacgaacagctgctgcgtggaatgcgcgcgatgctgaagacgcgtctgttcgatgctcgcatgttgactgcgcagcgccaaaaaaaattgagtttttatatgcagtgcttaggagaagaggcaatcgcgactgcccatacactggccctgcgcgatggtgatatgtgttttccgacgtaccgtcagcaggggattcttattacacgtgagtatccgcttgtggatatgatctgccagctgctgtcgaatgaagcggaccccctgaaaggccgtcaactgccgatcatgtacagcagtaaggaggctggcttctttagcatctcgggcaatcttgcgactcagtttattcaggcggtggggtgggggatggcaagcgcaatcaaaggggatacccgcattgcatccgcatggattggcgatggcgctaccgcggaaagcgattttcatacggcgctgacctttgctcacgtttatcgcgcaccggtgatcctcaatgtggtcaacaaccagtgggcgatttcgacgtttcaggccatcgcgggcggcgagggcaccacgttcgcgaaccgtggcgtgggttgcggcattgcgagcctccgtgtggacgggaacgattttttggccgtgtatgcggcgagcgaatgggcggcagaacgcgcacgccgtaacttgggaccgtccctgatcgaatgggtaacttatcgcgcgggcccacacagcacgagcgacgatccgtcaaagtatcgccctgcggatgattggaccaattttccgctgggtgacccgattgcgcgtctgaaacgtcacatgatcggtttgggtatttggagcgaagaacagcacgaagctacgcacaaagcgctggaagcggaagtcctggcggcgcagaagcaggccgaaagccatggcactctgattgacggccgtgtgccgtctgcagcctctatgttcgaagatgtttatgccgagttacccgagcacttacgtcgccagcgccaggagctcggggtatgaacgccatgaacccgcagcatgaaaacgcgcaaaccgtgacctccatgacgatgattcaggccctgcgctcggcgatggatattatgttagaacgtgacgatgacgtcgtggtgtttggtcaggacgtagggtattttgggggagtgtttcgttgtaccgaggggttgcaaaagaagtatggtacgagtcgcgtcttcgatgcaccgatcagcgaatcaggcattatcggcgctgccgtgggcatgggtgcatatggcttgcgccctgtggttgaaattcagtttgcagattatgtatatcccgcgtctgaccaactgattagtgaggcggcacgcctccgctaccgtagcgcgggcgatttcattgtcccgatgaccgtccgcatgccttgtggagggggcatttacggtggccaaacgcattctcagagtccagaagccatgttcacacaagtgtgcggtcttcgcaccgtgatgccatctaatccttatgacgccaaaggattactgattgcgtgcatcgaaaacgacgatccggttatctttttagaacccaaacgtctgtacaacggtcctttcgacggtcatcacgaccgtcctgtcacgccgtggagcaaacatccggcatcgcaagtcccggatgggtattataaagtgcctctggacaaagcagcgattgtccgccctggtgcagcccttacagtcctgacgtatggtaccatggtgtacgtggcgcaggccgcggcagatgaaaccggcctcgatgcggagattatcgacctccgcagtctgtggccgctggacttggaaactatcgtcgcgagtgtgaaaaagaccggtcgttgtgttattgcccatgaagcgactcgtacctgcggctttggcgccgaactgatgtccctggtgcaggaacactgttttcaccatcttgaggctccgattgaacgcgtcactggctgggacacaccgtaccctcatgcgcaggaatgggcctatttcccgggcccagcgcgcgtgggagccgcctttaaacgcgtgatggaggtctgaatgggtacccacgttattaaaatgcctgatattggtgaaggcatcgcggaggtagagctggttgaatggcacgttcaagtgggtgatagcgtgaatgaagatcaggtactcgcggaagtaatgacggacaaagcaacggttgaaatcccgtcccctgttgctggccgcatcttggcactgggtggccagccgggacaagttatggcggtgggaggagaattaattcgcctggaagtggagggtgccggaaacctggcggagtctccggccgcagctacgcccgccgctccggtggcagcaactccggaaaaacctaaagaagcaccggttgcagcgccaaaagcagctgccgaagcaccccgtgcgcttcgtgattctgaagcgccgcgccaacgccgccagccgggggaacgcccattagcatcaccggccgtccgtcagcgtgcccgcgacctgggaatcgagctgcagtttgttcagggctctggcccagccggccgcgtgcttcatgaggacctggatgcgtatcttacgcaggatggaagtgttgctcgttcaggcggcgctgcgcagggttacgcggaacgccatgatgaacaggcagtcccggtgatcggtctgcgccgcaaaattgcccagaagatgcaggatgctaaacgccgcattcctcacttcagttacgtcgaagagattgacgtaaccgatctggaagccctgcgcgctcacttgaatcagaaatggggcgggcaacgtggtaaactgacgctgctgcctttcctcgtccgcgcaatggtcgtcgcattacgcgatttcccgcaactgaatgctcgctatgatgatgaagcggaagtagtgacgcgttacggggccgttcatgttggtatcgcgacccagtcagataatgggctcatggttccggtgttgcgccatgcagaaagccgtgacctgtggggtaatgcgtcggaagttgcgcgtctggccgaagcggcgcgttccggtaaagcgcaacgtcaggaactgagcggctccacgattaccctgtcaagccttggtgtgttgggagggattgtatccacgccagtcattaatcacccggaagttgcaatcgttggtgttaaccgtattgtggagcgccctatggttgttggtggtaatattgtagtacgtaaaatgatgaatctgagctcttcgtttgatcatcgcgtggtggacggcatggatgctgcggcttttattcaagccgtgcgcggtttgttagaacatcctgccaccctgttcctggagtaagcgatgagtcagattttaaaaacctcgctcctgatcgttggcggcgggccaggcggctatgtggcggcgatccgcgccggccagctggggattccaacggtgttggttgagggcgccgctttgggcggtacttgcctgaatgtggggtgcattccgagcaaagcgttgatccatgctgccgaagagtaccttaaagcgcgccactatgcatcacgttccgcgctgggcatccaggtgcaagcaccttcaattgacatcgcccgcaccgtggaatggaaagacgccattgtggaccgtttgacttcgggtgtggcggctctgctgaaaaagcatggtgtggatgtagtacaaggatgggcacgcatcctcgacggcaagagcgtggcggttgaactggcgggcggggggtcgcagcgcatcgagtgtgaacatctgcttctggcggcgggctcacaaagcgttgaattacccatcctgcctctggggggcaaagtaatcagcagcaccgaagcattagctccggggtcgttgccaaaacgtctggtggttgtgggtggcggttatattggtctggagctgggcactgcatatcgcaagctgggtgttgaagttgctgtggtggaggcacaaccccgcatcctgccgggctacgatgaggaactgactaagccggtggcccaagcgctgcgccgtctgggtgtagaactgtacctgggtcattcattgctgggaccgagtgaaaacggcgttcgcgtgcgtgatggggctggcgaagaacgtgagatcgccgcggaccaggtccttgtcgcagttggccgcaaaccgcgttcagagggttggaacctggagtctctcggtttagacatgaatgggcgtgccgtaaaagtggacgatcagtgccgtacaagcatgcgtaacgtatgggccattggcgacctggcgggcgaaccgatgctggcgcaccgcgctatggcgcaaggagaaatggtcgccgaattgattgcgggcaaacgccgtcagtttgcgccggttgcaattcctgcagtctgttttacggatccggaagtggtggtggcgggtctgagtccggaacaggccaaagatgcgggtctggattgcctggtcgcgtcattcccgttcgcagccaacggccgcgccatgacgttggaagctaacgaaggctttgtccgcgtggtggcacgtcgtgacaaccatctggtggttggttggcaggcggtcggtaaagctgtgtctgaattaagcaccgcgttcgcacaatctctggaaatgggcgctcgcctcgaagacattgcaggcacaatccacgcgcaccccaccctgggtgaagctgttcaggaagcggcactccgtgccttaggtcacgccctgcacatttga

SEQ ID NO: 5 - DNA, 5597 bp, Artificial Sequence
  Synthetic: Tet-livKHMGF construct
ccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtgatcatcccaccgcccgtaaaatgcgggcgggtttagaaaggttaccttatgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataagatggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaa

SEQ ID NO: 6 - DNA, 4657 bp, Artificial Sequence
  Synthetic: pKIKO-lacZ
agattgcagcattacacgtcttgagcgattgtgtaggctggagctgcttcgaagttcctatactttctagagaataggaacttcggaataggaacttcatttaaatggcgcgccttacgccccgccctgccactcatcgcagtactgttgtattcattaagcatctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatggaccatggctaattccttgccgttttcatcatatttaatcagcgactgatccacccagtcccagacgaagccgccctgtaaacgggggtactgacgaaacgcctgccagtatttagcgaagccgccaagactgttacccatcgcgtgggcatattcgcaaaggatcagcgggcgcatttctccaggcagcgaaagccattttttgatggaccatttcggcaccgccgggaagggctggtcttcatccacgcgcgcgtacatcgggcaaataatatcggtggccgtggtgtcggctccgccgccttcatactgtaccgggcgggaaggatcgacagatttgatccagcgatacagcgcgtcgtgattagcgccgtggcctgattcattccccagcgaccagatgatcacactcgggtgattacgatcgcgctgcaccatccgcgttacgcgttcgctcatcgcgggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgccgcgaatcagcaacggcttgccgttcagcagcagcagaccattttcaatccgcacctcgcggaaaccgacgtcgcaggcttctgcttcaatcagcgtgccgtcggcggtgtgcagttcaaccactgcacgatagagattcgggatttcggcgctccacagttccggattttcaacgttcaggcgtagtgtgacgcgatcggcataaccgccacgctcatcgataatttcacccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgactgacgggctccaggagtcgtcgccaccaatccccatatggaaaccgtcgatattcagccatgtgccttcttccgcgtgcagcagatggcgatggctggtttccatcagttgttgttggctgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggctgcagtaaggcggtcgggatagttttcttgcggccccaggccgagccagtttacccgctctgagacctgcgccagctggcaggtcaggccaatccgcgccggatgcggtgtatcgcttgccaccgcaacatccacattgatgaccatctcaccgtgcccatcaatccggtaggttttccggctgataaataaggttttcccctgatgctgccacgcgtgggcggttgtaatcagcaccgcgtcggcaagtgtatctgccgtgcactgcaacaacgccgcttcggcctggtaatggcccgccgccttccagcgttcgacccaggcgttagggtcaatgcgggtcgcttcacttacgccaatgtcgttatccagcggcgcacgggtgaactgatcgcgcagcggggtcagcagttgtttttcatcgccaatccacatctgtgaaagaaagcctgactggcggttaaattgccaacgcttattacccagctcgatgcaaaaatccgttccgctggtggtcagttgagggatggcgtgggacgcggaggggagtgtcacgctgaggttttccgccagacgccattgctgccaggcgctgatgtgtccggcttctgaccatgcggtcgcgtttggttgcactacgcgtaccgttagccagagtcacatttccccgaaaagtgccacctgcatcgatggccccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 7 - DNA, 10254 bp, Artificial Sequence
  Synthetic: pTet-livKHMGF sequence
agattgcagcattacacgtcttgagcgattgtgtaggctggagctgcttcgaagttcctatactttctagagaataggaacttcggaataggaacttcatttaaatggcgcgccttacgccccgccctgccactcatcgcagtactgttgtattcattaagcatctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatggaccatggctaattccttgccgttttcatcatatttaatcagcgactgatccacccagtcccagacgaagccgccctgtaaacgggggtactgacgaaacgcctgccagtatttagcgaagccgccaagactgttacccatcgcgtgggcatattcgcaaaggatcagcgggcgcatttctccaggcagcgaaagccattttttgatggaccatttcggcaccgccgggaagggctggtcttcatccacgcgcgcgtacatcgggcaaataatatcggtggccgtggtgtcggctccgccgccttcatactgtaccgggcgggaaggatcgacagatttgatccagcgatacagcgcgtcgtgattagcgccgtggcctgattcattccccagcgaccagatgatcacactcgggtgattacgatcgcgctgcaccatccgcgttacgcgttcgctcatcgcgggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgccgcgaatcagcaacggcttgccgttcagcagcagcagaccattttcaatccgcacctcgcggaaaccgacgtcgcaggcttctgcttcaatcagcgtgccgtcggcggtgtgcagttcaaccactgcacgatagagattcgggatttcggcgctccacagttccggattttcaacgttcaggcgtagtgtgacgcgatcggcataaccgccacgctcatcgataatttcacccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgactgacgggctccaggagtcgtcgccaccaatccccatatggaaaccgtcgatattcagccatgtgccttcttccgcgtgcagcagatggcgatggctggtttccatcagttgttgttggctgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggctgcagtaaggcggtcgggatagttttcttgcggccccaggccgagccagtttacccgctctgagacctgcgccagctggcaggtcaggccaatccgcgccggatgcggtgtatcgcttgccaccgcaacatccacattgatgaccatctcaccgtgcccatcaatccggtaggttttccggctgataaataaggttttcccctgatgctgccacgcgtgggcggttgtaatcagcaccgcgtcggcaagtgtatctgccgtgcactgcaacaacgccgcttcggcctggtaatggcccgccgccttccagcgttcgacccaggcgttagggtcaatgcgggtcgcttcacttacgccaatgtcgttatccagcggcgcacgggtgaactgatcgcgcagcggggtcagcagttgtttttcatcgccaatccacatctgtgaaagaaagcctgactggcggttaaattgccaacgcttattacccagctcgatgcaaaaatccgttccgctggtggtcagttgagggatggcgtgggacgcggaggggagtgtcacgctgaggttttccgccagacgccattgctgccaggcgctgatgtgtccggcttctgaccatgcggtcgcgtttggttgcactacgcgtaccgttagccagagtcacatttccccgaaaagtgccacctgcatcgatggccccccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtgatcatcccaccgcccgtaaaatgcgggcgggtttagaaaggttaccttatgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataagatggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 8 - DNA, 639 bp, E. coli
  misc_feature: E. coli Nissle 1917 leucine exporter gene leuE
gtgttcgctgaatacggggttctgaattactggacctatctggttggggccatttttattgtgttggtgccagggccaaataccctgtttgtactcaaaaatagcgtcagtagcggtatgaaaggcggttatcttgcggcctgtggtgtatttattggcgatgcggtattgatgtttctggcatgggctggagtggcgacattaattaagaccaccccgatattattcaacatcgtacgttatcttggtgcgttttatttgctctatctggggagtaaaattctctacgcgaccctgaaaggtaaaaatagcgagaccaaatccgatgagccccaatacggtgccatttttaaacgcgcgttaattttgagcctgactaatccgaaagccattttgttctatgtgtcgtttttcgtacagtttatcgatgttaatgccccacatacgggaatttcattctttattctggcgacgacgctggaactggtgagtttctgctatttgagcttcctgattatttctggggcttttgtcacgcagtacatacgtaccaaaaagaaactggctaaagtgggcaactcactgattggtttgatgttcgtgggtttcgccgcccgactggcgacgctgcaatcctga

SEQ ID NO: 9 - DNA, 1707 bp, Artificial Sequence
  Synthetic: leuE deletion construct
cattttaaataccatttattggttactttttagcaccatatcagcgaagaatcagggaggattatagatgggaagcccatgcagattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggaccatggctaattcccaattaacctctttaattatctttcgatcatgcgcgattaaaggtgaatatgctaaccaatctgtagcggcttagaaaggagaaaatcaggttttaacctga

SEQ ID NO: 10 - DNA, 8864 bp, Artificial Sequence
  Synthetic: Tet-livKHMGF fragment
aataggggttccgcgactgacgggctccaggagtcgtcgccaccaatccccatatggaaaccgtcgatattcagccatgtgccttcttccgcgtgcagcagatggcgatggctggtttccatcagttgttgttggctgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggctgcagtaaggcggtcgggatagttttcttgcggccccaggccgagccagtttacccgctctgagacctgcgccagctggcaggtcaggccaatccgcgccggatgcggtgtatcgcttgccaccgcaacatccacattgatgaccatctcaccgtgcccatcaatccggtaggttttccggctgataaataaggttttcccctgatgctgccacgcgtgggcggttgtaatcagcaccgcgtcggcaagtgtatctgccgtgcactgcaacaacgccgcttcggcctggtaatggcccgccgccttccagcgttcgacccaggcgttagggtcaatgcgggtcgcttcacttacgccaatgtcgttatccagcggcgcacgggtgaactgatcgcgcagcggggtcagcagttgtttttcatcgccaatccacatctgtgaaagaaagcctgactggcggttaaattgccaacgcttattacccagctcgatgcaaaaatccgttccgctggtggtcagttgagggatggcgtgggacgcggaggggagtgtcacgctgaggttttccgccagacgccattgctgccaggcgctgatgtgtccggcttctgaccatgcggtcgcgtttggttgcactacgcgtaccgttagccagagtcacatttccccgaaaagtgccacctgcatcgatggccccccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtgatcatcccaccgcccgtaaaatgcgggcgggtttagaaaggttaccttatgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataagatggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaacttcatttaaatggcgcgccttacgccccgccctgccactcatcgcagtactgttgtattcattaagcatctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatggaccatggctaattccttgccgttttcatcatatttaatcagcgactgatccacccagtcccagacgaagccgccctgtaaacgggggtactgacgaaacgcctgccagtatttagcgaagccgccaagactgttacccatcgcgtgggcatattcgcaaaggatcagcgggcgcatttctccaggcagcgaaagccattttttgatggaccatttcggcaccgccgggaagggctggtcttcatccacgcgcgcgtacatcgggcaaataatatcggtggccgtggtgtcggctccgccgccttcatactgtaccgggcgggaaggatcgacagatttgatccagcgatacagcgcgtcgtgattagcgccgtggcctgattcattccccagcgaccagatgatcacactcgggtgattacgatcgcgctgcaccatccgcgttacgcgttcgctcatcgcgggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgccgcgaatcagcaacggcttgccgttcagcagcagcagaccattttcaatccgcacctcgcggaaaccgacgtcgcaggcttctgcttcaatcagcgtgccgtcggcggtgtgcagttcaaccactgcacgatagagattcgggatttcggcgctccacagttccggattttcaacgttcaggcgtagtgtgacgcgatcggcataaccgccacgctcatcgataatttcacccatgtcagccgttaag

SEQ ID NO: 11 - DNA, 2344 bp, Artificial Sequence
  Synthetic: Ptac-livJ construct
agacaacaagtccacgttgcaggaactggctgaccgttacggtgtttccgctgagcgtgtgcgtcagctggaaaagaacgcgatgaaaaaattgcgcgctgccattgaagcgtaatttccgctattaagcagagaaccctggatgagagtccggggtttttgttttttgggcctctacaataatcaattccccctccggcaaaacgccaatccccacgcagattgttaataaactgtcaaaatagctataacacatttccccgaaaagtgccgatggccccccgatggtagtgtggcccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggaccatggctaattcccatgttgacaattaatcatcggctcgtataatgttagcagagtatgctgctaaagcacgggtagctacgtataaaacgaaataaagtgctgcacaacaacatcacaacacacgtaataaccagaagagtggggattctcaggatgaacataaagggtaaagcgttactggcaggatgtatcgcgctggcattcagcaatatggctctggcagaagatattaaagtcgccgtcgtaggcgcaatgtccggtccggtggcgcag

SEQ ID NO: 12 - DNA, 1104 bp, Artificial Sequence
  Synthetic: livJ sequence
atgaacataaagggtaaagcgttactggcaggatgtatcgcgctggcattcagcaatatggctctggcagaagatattaaagtcgcggtcgtgggcgcaatgtccggtccggttgcgcagtacggtgaccaggagtttaccggcgcagagcaggcggttgcggatatcaacgctaaaggcggcattaaaggcaacaaactgcaaatcgtaaaatatgacgatgcctgtgacccgaaacaggcggttgcggtggcgaacaaagtcgttaacgacggcattaaatatgtgattggtcacctctgttcttcatcaacgcagcctgcgtctgacatctacgaagacgaaggcattttaatgatcaccccagcggcaaccgcgccggagctgaccgcccgtggctatcagctgatcctgcgcaccaccggcctggactccgaccaggggccgacggcggcgaaatatattcttgagaaagtgaaaccgcagcgtattgctatcgttcacgacaaacagcaatacggcgaaggtctggcgcgagcggtgcaggacggcctgaagaaaggcaatgcaaacgtggtgttctttgatggcatcaccgccggggaaaaagatttctcaacgctggtggcgcgtctgaaaaaagagaatatcgacttcgtttactacggcggttatcacccggaaatggggcaaatcctgcgtcaggcacgcgcggcagggctgaaaactcagtttatggggccggaaggtgtggctaacgtttcgctgtctaacattgcgggcgaatcagcggaagggctgctggtgaccaagccgaagaactacgatcaggttccggcgaacaaacccattgttgacgcgatcaaagcgaaaaaacaggacccaagtggcgcattcgtttggaccacctacgccgcgctgcaatctttgcaggcgggcctgaatcagtctgacgatccggctgaaatcgccaaatacctgaaagcgaactccgtggataccgtaatgggaccgctgacctgggatgagaaaggcgatctgaaaggctttgagttcggcgtatttgactggcacgccaacggcacggcgaccgatgcgaagtaa

SEQ ID NO: 13 - DNA, 1921 bp, Artificial Sequence
  Synthetic: Prp promoter
ttacccgtctggattttcagtacgcgcttttaaacgacgccacagcgtggtacggctgatccccaaataacgtgcggcggcgcgcttatcgccattaaagcgtgcgagcacctcctgcaatggaagcgcttctgctgacgagggcgtgatttctgctgtggtccccaccagttcaggtaataattgccgcataaattgtctgtccagtgttggtgcgggatcgacgcttaaaaaaagcgccaggcgttccatcatattccgcagttcgcgaatattaccgggccaatgatagttcagtagaagcggctgacactgcgtcagcccatgacgcaccgattcggtaaaagggatctccatcgcggccagcgattgttttaaaaagttttccgccagaggcagaatatcaggctgtcgctcgcgcaaggggggaagcggcagacgcagaatgctcaaacggtaaaacagatcggtacgaaaacgtccttgcgttatctcccgatccagatcgcaatgcgtggcgctgatcacccggacatctaccgggatcggctgatgcccgccaacgcgggtgacggctttttcctccagtacgcgtagaaggcgggtttgtaacggcagcggcatttcgccaatttcgtcaagaaacagcgtgccgccgtgggcgacctcaaacagccccgcacgtccacctcgtcttgagccggtaaacgctccctcctcatagccaaacagttcagcctccagcaacgactcggtaatcgcgccgcaattaacggcgacaaagggcggagaaggcttgttctgacggtggggctgacggttaaacaacgcctgatgaatcgcttgcgccgccagctctttcccggtccctgtttccccctgaatcagcactgccgcgcgggaacgggcatagagtgtaatcgtatggcgaacctgctccatttgtggtgaatcgccgaggatatcgctcagcgcataacgggtctgtaatcccttgctggaggtatgctggctatactgacgccgtgtcaggcgggtcatatccagcgcatcatggaaagcctgacgtacggtggccgctgaataaataaagatggcggtcattcctgcctcttccgccaggtcggtaattagtcctgccccaattacagcctcaatgccgttagctttgagctcgttaatttgcccgcgagcatcctcttcagtgatatagcttcgctgttcaagacggaggtgaaacgttttctgaaaggcgaccagagccggaatggtctcctgataggtcacgattcccattgaggaagtcagctttcccgcttttgccagagcctgtaatacatcgaatccgctgggtttgatgaggatgacaggtaccgacagtcggctttttaaataagcgccgttggaacctgccgcgataatcgcgtcgcagcgttcggttgccagttttttgcgaatgtaggctactgccttttcaaaaccgagctgaataggcgtgatcgtcgccagatgatcaaactccaggctgatatcccgaaatagttcgaacaggcgcgttaccgagaccgtccagatcaccggtttatcgctattatcgcgcgaagcgctatgcacagtaaccatcgtcgtagattcatgtttaaggaacgaattcttgttttatagatgtttcgttaatgttgcaatgaaacacaggcctccgtttcatgaaacgttagctgactcgtttttcttgtgactcgtctgtcagtattaaaaaagatttttcatttaactgattgtttttaaattgaattttatttaatggtttctcggtttttgggtctggcatatcccttgctttaatgagtgcatcttaattaacaattcaataacaagagggctgaatagtaatttcaacaaaataacgagcattcgaatg

SEQ ID NO: 14 - DNA, 290 bp, Artificial Sequence
  Synthetic: FNR Responsive Promoter
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 15 - DNA, 173 bp, Artificial Sequence
  Synthetic: FNR Responsive Promoter
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 16 - DNA, 305 bp, Artificial Sequence
  Synthetic: FNR Responsive Promoter
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 17 - DNA, 180 bp, Artificial Sequence
  Synthetic: FNR Responsive Promoter
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 18 - DNA, 199 bp, Artificial Sequence
  Synthetic: FNR Responsive Promoter
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 19 - protein, 341 aa, Pseudomonas aeruginosa PA01
  misc_feature: LeuDH Amino acid sequence;  Leucine dehydrogenase LeuDH
MFDMMDAARLEGLHLAQDPATGLKAIIAIHSTRLGPALGGCRYLPYPNDEAAIGDAIRLAQGMSYKAALAGLEQGGGKAVIIRPPHLDNRGALFEAFGRFIESLGGRYITAVDSGTSSADMDCIAQQTRHVTSTTQAGDPSPHTALGVFAGIRASAQARLGSDDLEGLRVAVQGLGHVGYALAEQLAAVGAELLVCDLDPGRVQLAVEQLGAHPLAPEALLSTPCDILAPCGLGGVLTSQSVSQLRCAAVAGAANNQLERPEVADELEARGILYAPDYVINSGGLIYVALKHRGADPHSITAHLARIPARLTEIYAHAQADHQSPARIADRLAERILYGPQ

SEQ ID NO: 20 - DNA, 1026 bp, Artificial Sequence
  Synthetic: leuDH codon-optimized nucleotide sequence
atgttcgacatgatggatgcagcccgcctggaaggcctgcacctcgcccaggatccagcgacgggcctgaaagcgatcatcgcgatccattccactcgcctcggcccggccttaggcggctgtcgttacctcccatatccgaatgatgaagcggccatcggcgatgccattcgcctggcgcagggcatgtcctacaaagctgcacttgcgggtctggaacaaggtggtggcaaggcggtgatcattcgcccaccccacttggataatcgcggtgccttgtttgaagcgtttggacgctttattgaaagcctgggtggccgttatatcaccgccgttgactcaggaacaagtagtgccgatatggattgcatcgcccaacagacgcgccatgtgacttcaacgacacaagccggcgacccatctccacatacggctctgggcgtctttgccggcatccgcgcctccgcgcaggctcgcctggggtccgatgacctggaaggcctgcgtgtcgcggttcagggccttggccacgtaggttatgcgttagcggagcagctggcggcggtcggcgcagaactgctggtgtgcgacctggaccccggccgcgtccagttagcggtggagcaactgggggcgcacccactggcccctgaagcattgctctctactccgtgcgacattttagcgccttgtggcctgggcggcgtgctcaccagccagtcggtgtcacagttgcgctgcgcggccgttgcaggcgcagcgaacaatcaactggagcgcccggaagttgcagacgaactggaggcgcgcgggattttatatgcgcccgattacgtgattaactcgggaggactgatttatgtggcgctgaagcatcgcggtgctgatccgcatagcattaccgcccacctcgctcgcatccctgcacgcctgacggaaatctatgcgcatgcgcaggcggatcatcagtcgcctgcgcgcatcgccgatcgtctggcggagcgcattctgtacggcccgcagtga

SEQ ID NO: 21 - protein, 360 aa, E. coli Nissle
  misc_feature: IlvE Amino acid sequence;  Branched-chain amino acid aminotransferase IlvE
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 22 - DNA, 930 bp, Artificial Sequence
  Synthetic: ilvE nucleotide sequence
atgaccacgaagaaagctgattacatttggttcaatggggagatggttcgctgggaagacgcgaaggtgcatgtgatgtcgcacgcgctgcactatggcacctcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgttggtgatgttggcatgggcgtaaacccgccagcgggatactcaaccgacgtgattatcgccgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgttggatgtgaatggttacatctctgaaggcgcaggcgaaaacctgtttgaagtgaaagacggcgtgctgttcaccccaccgttcacctcatccgcgctgccgggtattacccgtgatgccatcatcaaactggcaaaagagctgggaattgaagtgcgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataa

SEQ ID NO: 23 - protein, 471 aa, Proteus vulgaris
  misc_feature: L-AAD Amino acid sequence
MAISRRKFIIGGTVVAVAAGAGILTPMLTREGRFVPGTPRHGFVEGTEGALPKQADVVVVGAGILGIMTAINLVERGLSVVIVEKGNIAGEQSSRFYGQAISYKMPDETFLLHHLGKHRWREMNAKVGIDTTYRTQGRVEVPLDEEDLVNVRKWIDERSKNVGSDIPFKTRIIEGAELNQRLRGATTDWKIAGFEEDSGSFDPEVATFVMAEYAKKMGVRIYTQCAARGLETQAGVISDVVTEKGAIKTSQVVVAGGVWSRLFMQNLNVDVPTLPAYQSQQLISGSPTAPGGNVALPGGIFFREQADGTYATSPRVIVAPVVKESFTYGYKYLPLLALPDFPVHISLNEQLINSFMQSTHWNLDEVSPFEQFRNMTALPDLPELNASLEKLKAEFPAFKESKLIDQWSGAMAIAPDENPIISEVKEYPGLVINTATGWGMTESPVSAELTADLLLGKKPVLDPKPFSLYRF

SEQ ID NO: 24 - DNA, 1416 bp, Artificial Sequence
  Synthetic: L-AAD Codon-optimized nucleotide sequence
atggccatcagtcgtcgcaaattcattatcggtggaacggtcgtcgccgttgccgccggtgcggggattttgaccccgatgctgacgcgcgaagggcgctttgtgccgggcactccacgccacggtttcgttgaagggaccgagggggctttacccaaacaagcggacgtggtggtcgtaggcgctggaattcttggtattatgacggccattaatttggttgagcgtgggctgtcagtggtaattgtggagaagggcaatatcgcgggagaacaaagctctcgcttctacggacaggcaattagctataagatgccagatgagacatttttgctgcaccatcttgggaagcaccgctggcgtgagatgaatgcgaaagtagggattgatacaacgtaccgtactcaaggacgcgtggaagtaccgcttgacgaggaagatttggtaaacgtccgcaaatggattgacgaacgttcaaaaaatgttggatctgacattccttttaagacccgcattatcgagggggcagaattaaatcagcgtctgcgcggcgccacaacagattggaagatcgctggcttcgaggaggacagcgggtcattcgatcccgaggtagcgacctttgtaatggcagagtacgcgaagaagatgggtgttcgtatctatacgcaatgcgcggcccgcggtctggaaacccaggccggtgtcatttcagatgttgtcacggaaaaaggtgcgattaagacctcccaagtggtagtggctggtggggtgtggagtcgtctgttcatgcagaatttaaacgtcgacgtcccaacccttcctgcgtatcagtcacagcagttgattagtggttcccctaccgcaccgggggggaacgtcgcattacctggtggaatcttcttccgcgaacaggcggacgggacatacgcgacttctcctcgtgtgattgttgccccagttgtgaaggagagcttcacttatggttacaagtacttaccattattagcattgcctgatttccctgttcacattagcctgaatgaacagttaattaattcgtttatgcaaagtacccactggaacttagacgaagtgtcgccgttcgaacaatttcgcaacatgacagcattacctgacttgcccgaacttaacgccagcttagaaaagttaaaggcagagttccctgctttcaaagaatccaagttgatcgaccagtggtctggagcaatggcaattgcgcccgacgaaaatccaatcatttccgaggtgaaggagtacccaggtctggtaattaacacggcgacaggttggggcatgactgaaagtccagtgtctgctgaacttaccgccgatcttctgctggggaagaagccggtgttagatcctaagccattctcactttatcgcttttga

SEQ ID NO: 25 - protein, 471 aa, Proteus mirabilis
  misc_feature: L-AAD Amino acid sequence
MAISRRKFILGGTVVAVAAGAGVLTPMLTREGRFVPGTPRHGFVEGTGGPLPKQDDVVVIGAGILGIMTAINLAERGLSVTIVEKGNIAGEQSSRFYGQAISYKMPDETFLLHHLGKHRWREMNAKVGIDTTYRTQGRVEVPLDEEDLENVRKWIDAKSKDVGSDIPFRTKMIEGAELKQRLRGATTDWKIAGFEEDSGSFDPEVATFVMAEYAKKMGIKIFTNCAARGLETQAGVISDVVTEKGPIKTSRVVVAGGVGSRLFMQNLNVDVPTLPAYQSQQLISAAPNAPGGNVALPGGIFFRDQADGTYATSPRVIVAPVVKESFTYGYKYLPLLALPDFPVHISLNEQLINSFMQSTHWDLNEESPFEKYRDMTALPDLPELNASLEKLKKEFPAFKESTLIDQWSGAMAIAPDENPIISDVKEYPGLVINTATGWGMTESPVSAEITADLLLGKKPVLDAKPFSLYRF

SEQ ID NO: 26 - DNA, 1416 bp, Artificial Sequence
  Synthetic: L-AAD Nucleotide sequence
atggcaataagtagaagaaaatttattcttggtggcacagtggttgctgttgctgcaggcgctggggttttaacacctatgttaacgcgagaagggcgttttgttcctggtacgccgagacatggttttgttgagggaactggcggtccattaccgaaacaagatgatgttgttgtaattggtgcgggtattttaggtatcatgaccgcgattaaccttgctgagcgtggcttatctgtcacaatcgttgaaaaaggaaatattgccggcgaacaatcatctcgattctatggtcaagctattagctataaaatgccagatgaaaccttcttattacatcacctcgggaagcaccgctggcgtgagatgaacgctaaagttggtattgataccacttatcgtacacaaggtcgtgtagaagttcctttagatgaagaagatttagaaaacgtaagaaaatggattgatgctaaaagcaaagatgttggctcagacattccatttagaacaaaaatgattgaaggcgctgagttaaaacagcgtttacgtggcgctaccactgattggaaaattgctggtttcgaagaagactcaggaagcttcgatcctgaagttgcgacttttgtgatggcagaatatgccaaaaaaatgggtatcaaaattttcacaaactgtgcagcccgtggtttagaaacgcaagctggtgttatttctgatgttgtaacagaaaaaggaccaattaaaacctctcgtgttgttgtcgccggtggtgttgggtcacgtttatttatgcagaacctaaatgttgatgtaccaacattacctgcttatcaatcacagcaattaattagcgcagcaccaaatgcgccaggtggaaacgttgctttacccggcggaattttctttcgtgatcaagcggatggaacgtatgcaacttctcctcgtgtcattgttgctccggtagtaaaagaatcatttacttacggctataaatatttacctctgctggctttacctgatttcccagtacatatttcgttaaatgagcagttgattaattcctttatgcaatcaacacattgggatcttaatgaagagtcgccatttgaaaaatatcgtgatatgaccgctctgcctgatctgccagaattaaatgcctcactggaaaaactgaaaaaagagttcccagcatttaaagaatcaacgttaattgatcagtggagtggtgcgatggcgattgcaccagatgaaaacccaattatctctgatgttaaagagtatccaggtctagttattaatactgcaacaggttggggaatgactgaaagccctgtatcagcagaaattacagcagatttattattaggcaaaaaaccagtattagatgccaaaccatttagtctgtatcgtttctaa

SEQ ID NO: 27 - protein, 548 aa, lactococcus lactis strain IFPL730
  misc_feature: KivD Amino acid sequence
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 28 - DNA, 1647 bp, Artificial Sequence; identical to SEQ ID NO: 1
  Synthetic: kivD Nucleotide sequence

SEQ ID NO: 29 - DNA, 1647 bp, Artificial Sequence
  Synthetic: kivD Codon-optimized sequence
atgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataa

SEQ ID NO: 30 - protein, 548 aa, lactococcus lactis strain B1157
  misc_feature: KdcA Amino acid sequence
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNKS

SEQ ID NO: 31 - DNA, 1647 bp, Artificial Sequence
  Synthetic: kdcA Nucleotide sequence
atgtatacagtaggagattacctattagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggcaaactatttgctgaacaaaataaatcataa

SEQ ID NO: 32 - DNA, 1647 bp, Artificial Sequence
  Synthetic: kdcA Codon-optimized kdcA sequence
atgtatacagtaggagattaccttttagatcgtttgcacgaattgggcattgaggaaatttttggcgtccctggcgactacaatttacaattcttagatcagattatttcacgtgaggatatgaagtggattgggaatgccaatgagctgaacgcgagctatatggcggacggttacgctcgtacaaaaaaggcagcagcgtttcttactacttttggcgtaggcgaattgtcggccatcaacgggcttgcgggttcgtatgcggaaaacttaccggttgtcgagattgtcggttcccctacttcgaaggtgcagaatgatggcaaattcgttcatcacaccttggcagacggcgactttaaacatttcatgaaaatgcacgaacctgtgactgccgcccgcacacttctgacagctgaaaacgcgacatacgaaattgatcgcgtgctttcgcagttgttgaaagagcgtaaacccgtatatatcaatctgccggtggatgtagcggctgcaaaagccgaaaaaccggcgctgtcactggaaaaagaatcgtctacgactaatacaacggaacaagtaatcctgtcaaaaatcgaagagagcttgaaaaacgcccagaagcctgtcgtgattgccgggcacgaggtcattagttttgggttagaaaagactgttacccagttcgtgagtgagacgaagttgcccatcaccacccttaactttggcaagtctgcggtagacgagagcttaccgtcttttttaggtatctacaatgggaaactttcagaaatttcactgaaaaacttcgtggagtcggcagactttattttaatgttgggtgttaaattaactgatagcagcactggcgcgttcacgcatcacttggatgagaataaaatgatctcgcttaacatcgacgaaggtatcatttttaataaagttgtagaggacttcgactttcgtgctgttgtatcgagcctttccgaattaaagggtatcgagtacgaaggtcagtacattgacaagcaatacgaggaatttatcccctccagcgcgcctcttagccaagaccgcctttggcaggccgtagagagtcttacacaaagtaatgaaactattgttgcagaacagggtacaagcttctttggcgcctcgacgattttcttaaaatcgaacagtcgctttatcgggcaacctctttgggggtcgattgggtacacctttcctgcggccttaggctctcaaattgcggacaaagaatctcgccatttattattcatcggcgacggctcgttacagcttacagtgcaagagttgggattatcgattcgcgagaagctgaatccgatttgctttatcattaacaacgacgggtacacagtcgaacgcgaaatccatggcccgacacaatcatataatgacatccctatgtggaattattctaagcttccagagacattcggcgcaactgaagaccgcgtcgtgtcaaaaattgtccgcactgagaatgaattcgtgtcagtgatgaaggaagctcaggccgatgtcaaccgcatgtactggattgaattagttttggagaaagaggatgcccccaaattacttaagaagatggggaaactatttgctgaacaaaataaatcataa

SEQ ID NO: 33 - protein, 609 aa, Saccharomyces cerevisiae
  misc_feature: THI3/KID1 Amino acid sequence
MNSSYTQRYALPKCIAISDYLFHRLNQLNIHTIFGLSGEFSMPLLDKLYNIPNLRWAGNSNELNAAYAADGYSRLKGLGCLITTFGVGELSAINGVAGSYAEHVGILHIVGMPPTSAQTKQLLLHHTLGNGDFTVFHRIASDVACYTTLIIDSELCADEVDKCIKKAWIEQRPVYMGMPVNQVNLPIESARLNTPLDLQLHKNDPDVEKEVISRILSFIYKSQNPAIIVDACTSRQNLIEETKELCNRLKFPVFVTPMGKGTVNETDPQFGGVFTGSISAPEVREVVDFADFIIVIGCMLSEFSTSTFHFQYKTKNCALLYSTSVKLKNATYPDLSIKLLLQKILANLDESKLSYQPSEQPSMMVPRPYPAGNVLLRQEWVWNEISHWFQPGDIIITETGASAFGVNQTRFPVNTLGISQALWGSVGYTMGACLGAEFAVQEINKDKFPATKHRVILFMGDGAFQLTVQELSTIVKWGLTPYIFVMNNQGYSVDRFLHHRSDASYYDIQPWNYLGLLRVFGCTNYETKKIITVGEFRSMISDPNFATNDKIRMIEIMLPPRDVPQALLDRWVVEKEQSKQVQEENENSSAVNTPTPEFQPLLKKNQVGY

SEQ ID NO: 34 - DNA, 1830 bp, Artificial Sequence
  Synthetic: THI3/KID1 Nucleotide sequence
atgaattctagctatacacagagatatgcactgccgaagtgtatagcaatatcagattatcttttccatcggctcaaccagctgaacatacataccatatttggactctccggagaatttagcatgccgttgctggataaactatacaacattccgaacttacgatgggccggtaattctaatgagttaaatgctgcctacgcagcagatggatactcacgactaaaaggcttgggatgtctcataacaacctttggtgtaggcgaattatcggcaatcaatggcgtggccggatcttacgctgaacatgtaggaatacttcacatagtgggtatgccgccaacaagtgcacaaacgaaacaactactactgcatcatactctgggcaatggtgatttcacggtatttcatagaatagccagtgatgtagcatgctatacaacattgattattgactctgaattatgtgccgacgaagtcgataagtgcatcaaaaaggcttggatagaacagaggccagtatacatgggcatgcctgtcaaccaggtaaatctcccgattgaatcagcaaggcttaatacacctctggatttacaattgcataaaaacgacccagacgtagagaaagaagttatttctcgaatattgagttttatatacaaaagccagaatccggcaatcatcgtagatgcatgtactagtcgacagaatttaatcgaggagactaaagagctttgtaataggcttaaatttccagtttttgttacacctatgggtaagggtacagtaaacgaaacagacccgcaatttgggggcgtattcacgggctcgatatcagccccagaagtaagagaagtagttgattttgccgattttatcatcgtcattggttgcatgctctccgaattcagcacgtcaactttccacttccaatataaaactaagaattgtgcgctactatattctacatctgtgaaattgaaaaatgccacatatcctgacttgagcattaaattactactacagaaaatattagcaaatcttgatgaatctaaactgtcttaccaaccaagcgaacaacccagtatgatggttccaagaccttacccagcaggaaatgtcctcttgagacaagaatgggtctggaatgaaatatcccattggttccaaccaggtgacataatcataacagaaactggtgcttctgcatttggagttaaccagaccagatttccggtaaatacactaggtatttcgcaagctctttggggatctgtcggatatacaatgggggcgtgtcttggggcagaatttgctgttcaagagataaacaaggataaattccccgcaactaaacatagagttattctgtttatgggtgacggtgctttccaattgacagttcaagaattatccacaattgttaagtggggattgacaccttatatttttgtgatgaataaccaaggttactctgtggacaggtttttgcatcacaggtcagatgctagttattacgatatccaaccttggaactacttgggattattgcgagtatttggttgcacgaactacgaaacgaaaaaaattattactgttggagaattcagatccatgatcagtgacccaaactttgcgaccaatgacaaaattcggatgatagagattatgctaccaccaagggatgttccacaggctctgcttgacaggtgggtggtagaaaaagaacagagcaaacaagtgcaagaggagaacgaaaattctagcgcagtaaatacgccaactccagaattccaaccacttctaaaaaaaaatcaagttggatactga

SEQ ID NO: 35 - protein, 635 aa, Saccharomyces cerevisiae
  misc_feature: ARO10 Amino acid sequence
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 36 - DNA, 1908 bp, Artificial Sequence
  Synthetic: ARO10 Nucleotide sequence
atggcacctgttacaattgaaaagttcgtaaatcaagaagaacgacaccttgtttccaaccgatcagcaacaattccgtttggtgaatacatatttaaaagattgttgtccatcgatacgaaatcagttttcggtgttcctggtgacttcaacttatctctattagaatatctctattcacctagtgttgaatcagctggcctaagatgggtcggcacgtgtaatgaactgaacgccgcttatgcggccgacggatattcccgttactctaataagattggctgtttaataaccacgtatggcgttggtgaattaagcgccttgaacggtatagccggttcgttcgctgaaaatgtcaaagttttgcacattgttggtgtggccaagtccatagattcgcgttcaagtaactttagtgatcggaacctacatcatttggtcccacagctacatgattcaaattttaaagggccaaatcataaagtatatcatgatatggtaaaagatagagtcgcttgctcggtagcctacttggaggatattgaaactgcatgtgaccaagtcgataatgttatccgcgatatttacaagtattctaaacctggttatatttttgttcctgcagattttgcggatatgtctgttacatgtgataatttggttaatgttccacgtatatctcaacaagattgtatagtatacccttctgaaaaccaattgtctgacataatcaacaagattactagttggatatattccagtaaaacacctgcgatccttggagacgtactgactgataggtatggtgtgagtaactttttgaacaagcttatctgcaaaactgggatttggaatttttccactgttatgggaaaatctgtaattgatgagtcaaacccaacttatatgggtcaatataatggtaaagaaggtttaaaacaagtctatgaacattttgaactgtgcgacttggtcttgcattttggagtcgacatcaatgaaattaataatgggcattatacttttacttataaaccaaatgctaaaatcattcaatttcatccgaattatattcgccttgtggacactaggcagggcaatgagcaaatgttcaaaggaatcaattttgcccctattttaaaagaactatacaagcgcattgacgtttctaaactttctttgcaatatgattcaaatgtaactcaatatacgaacgaaacaatgcggttagaagatcctaccaatggacaatcaagcattattacacaagttcacttacaaaagacgatgcctaaatttttgaaccctggtgatgttgtcgtttgtgaaacaggctcttttcaattctctgttcgtgatttcgcgtttccttcgcaattaaaatatatatcgcaaggatttttcctttccattggcatggcccttcctgccgccctaggtgttggaattgccatgcaagaccactcaaacgctcacatcaatggtggcaacgtaaaagaggactataagccaagattaattttgtttgaaggtgacggtgcagcacagatgacaatccaagaactgagcaccattctgaagtgcaatattccactagaagttatcatttggaacaataacggctacactattgaaagagccatcatgggccctaccaggtcgtataacgacgttatgtcttggaaatggaccaaactatttgaagcattcggagacttcgacggaaagtatactaatagcactctcattcaatgtccctctaaattagcactgaaattggaggagcttaagaattcaaacaaaagaagcgggatagaacttttagaagtcaaattaggcgaattggatttccccgaacagctaaagtgcatggttgaagcagcggcacttaaaagaaataaaaaatag

SEQ ID NO: 37 - protein, 348 aa, Saccharomyces cerevisae
  misc_feature: Adh2 Amino acid sequence
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 38 - DNA, 1047 bp, Artificial Sequence
  Synthetic: adh2 Nucleotide sequence
atgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataa

SEQ ID NO: 39 - DNA, 1047 bp, Artificial Sequence
  Synthetic: adh2 Codon-optimized sequence
atgtctattccagaaacgcagaaagccatcatattttatgaatcgaacggaaaacttgagcacaaggacatccccgtcccgaagccaaaacctaatgagttgcttatcaacgttaagtattcgggcgtatgccacacagacttgcacgcatggcacggggattggcccttaccgactaagttgccgttagtgggcggacatgagggggcgggagtcgtagtgggaatgggagagaacgtgaagggttggaagattggagattatgctgggattaagtggttgaatgggagctgcatggcctgcgaatattgtgaacttggaaatgagagcaattgcccacatgctgacttgtccggttacacacatgacggttcattccaggaatatgctacggctgatgcagtccaagcagcgcatatcccgcaagggacggacttagcagaagtagcgcccattctttgcgctgggatcaccgtatataaagcgttaaagagcgcaaatttacgggccggacattgggcggcgatcagcggggccgcaggggggctgggcagcttggccgtccagtacgctaaagctatgggttatcgggttttgggcattgacggaggaccgggaaaggaggaattattcacgtccttgggaggagaggtattcattgactttaccaaggaaaaagatatcgtctctgctgtagtaaaggctaccaatggcggtgcccacggaatcataaatgtttcagtttctgaagcggcgatcgaagcgtccactagatattgccgtgcaaatgggacagtcgtacttgtaggacttccggctggcgccaaatgcagctccgatgtatttaatcatgtcgtgaagtcaatctctatcgttggttcatatgtaggaaaccgcgccgatactcgtgaggctcttgacttttttgccagaggcctggttaagtcccccataaaagttgttggcttatccagcttacccgaaatatacgagaagatggagaagggccagatcgcggggagatacgttgttgacacttctaaataa

SEQ ID NO: 40 - protein, 360 aa, Saccharomyces cerevisae; identical to SEQ ID NO: 21
  misc_feature: Adh6 Amino acid sequence

SEQ ID NO: 41 - DNA, 1083 bp, Artificial Sequence
  Synthetic: adh6 Codon-optimized sequence
atgtcataccctgaaaaattcgagggtatcgccattcagagtcacgaagattggaagaatcccaagaagaccaaatacgaccccaagccgttctatgaccatgatatcgacatcaaaatcgaggcatgtggtgtgtgtggcagtgatattcattgcgcagcgggccattgggggaacatgaagatgcctctggtagtaggacatgagatcgttggaaaggttgtgaaattgggtccgaaaagtaactccggtcttaaagtaggtcagcgtgttggggtcggggcgcaagttttcagttgcctggagtgtgatcgttgtaagaacgataacgagccgtactgcacaaagtttgtaacgacgtattcacagccatatgaggatgggtatgtttctcaagggggctatgcaaactacgtccgcgtacatgaacactttgtggtgcctattcctgagaacattccgtctcacttggccgctcctttgttgtgcggaggtcttaccgtctactcgccattggttcgcaatgggtgcggtccgggcaaaaaggtagggatcgttggccttggtggtatcggatctatgggaacgttaatcagtaaggcgatgggagctgagacctacgttatttcccgttcatcacgtaagcgtgaggatgcgatgaagatgggtgcagatcactacatcgcaacgttagaagagggagattggggcgaaaaatattttgacacttttgacttgattgtggtttgtgcatcgtcacttacagacattgactttaatattatgccaaaggcaatgaaggtaggtgggcgtattgtgtccatttctatcccggaacaacacgagatgctttctctgaaaccctacggacttaaagctgtgtccatttcgtacagtgcccttggatctatcaaggaactgaatcagctgctgaagcttgtttcggagaaagacattaagatttgggtggagacattgccagtgggggaggccggcgttcacgaggcgtttgaacgcatggagaagggagatgttcgctatcgcttcacgctggttggttatgataaagaattcagtgattag

SEQ ID NO: 42 - protein, 348 aa, Saccharomyces cerevisae
  misc_feature: Adh1 Amino acid sequence
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 43 - DNA, 1047 bp, Artificial Sequence
  Synthetic: adh1 Nucleotide sequence
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 44 - protein, 375 aa, Saccharomyces cerevisae [sequence omitted]

SEQ ID NO: 45 - DNA, 1128 bp, Artificial Sequence
  Synthetic: adh3 Nucleotide sequence
atgttgagaacgtcaacattgttcaccaggcgtgtccaaccaagcctattttctagaaacattcttagattgcaatccacagctgcaatccctaagactcaaaaaggtgtcatcttttatgagaataaggggaagctgcattacaaagatatccctgtccccgagcctaagccaaatgaaattttaatcaacgttaaatattctggtgtatgtcacaccgatttacatgcttggcacggcgattggccattacctgttaaactaccattagtaggtggtcatgaaggtgctggtgtagttgtcaaactaggttccaatgtcaagggctggaaagtcggtgatttagcaggtatcaaatggctgaacggttcttgtatgacatgcgaattctgtgaatcaggtcatgaatcaaattgtccagatgctgatttatctggttacactcatgatggttctttccaacaatttgcgaccgctgatgctattcaagccgccaaaattcaacagggtaccgacttggccgaagtagccccaatattatgtgctggtgttactgtatataaagcactaaaagaggcagacttgaaagctggtgactgggttgccatctctggtgctgcaggtggcttgggttccttggccgttcaatatgcaactgcgatgggttacagagttctaggtattgatgcaggtgaggaaaaggaaaaacttttcaagaaattggggggtgaagtattcatcgactttactaaaacaaagaatatggtttctgacattcaagaagctaccaaaggtggccctcatggtgtcattaacgtttccgtttctgaagccgctatttctctatctacggaatatgttagaccatgtggtaccgtcgttttggttggtttgcccgctaacgcctacgttaaatcagaggtattctctcatgtggtgaagtccatcaatatcaagggttcttatgttggtaacagagctgatacgagagaagccttagacttctttagcagaggtttgatcaaatcaccaatcaaaattgttggattatctgaattaccaaaggtttatgacttgatggaaaagggcaagattttgggtagatacgtcgtcgatactagtaaataa

SEQ ID NO: 46 - protein, 382 aa, Saccharomyces cerevisae
  misc_feature: Adh4 Amino acid sequence
MSSVTGFYIPPISFFGEGALEETADYIKNKDYKKALIVTDPGIAAIGLSGRVQKMLEERDLNVAIYDKTQPNPNIANVTAGLKVLKEQNSEIVVSIGGGSAHDNAKAIALLATNGGEIGDYEGVNQSKKAALPLFAINTTAGTASEMTRFTIISNEEKKIKMAIIDNNVTPAVAVNDPSTMFGLPPALTAATGLDALTHCIEAYVSTASNPITDACALKGIDLINESLVAAYKDGKDKKARTDMCYAEYLAGMAFNNASLGYVHALAHQLGGFYHLPHGVCNAVLLPHVQEANMQCPKAKKRLGEIALHFGASQEDPEETIKALHVLNRTMNIPRNLKELGVKTEDFEILAEHAMHDACHLTNPVQFTKEQVVAIIKKAYEY

SEQ ID NO: 47 - DNA, 1149 bp, Artificial Sequence
  Synthetic: adh4 Nucleotide sequence
atgtcttccgttactgggttttacattccaccaatctctttctttggtgaaggtgctttagaagaaaccgctgattacatcaaaaacaaggattacaaaaaggctttgatcgttactgatcctggtattgcagctattggtctctccggtagagtccaaaagatgttggaagaacgtgacttaaacgttgctatctatgacaaaactcaaccaaacccaaatattgccaatgtcacagctggtttgaaggttttgaaggaacaaaactctgaaattgttgtttccattggtggtggttctgctcacgacaatgctaaggccattgctttattggctactaacggtggggaaatcggagactatgaaggtgtcaatcaatctaagaaggctgctttaccactatttgccatcaacactactgctggtactgcttccgaaatgaccagattcactattatctctaatgaagaaaagaaaatcaagatggctatcattgacaacaacgtcactccagctgttgctgtcaacgatccatctaccatgtttggtttgccacctgctttgactgctgctactggtctagatgctttgactcactgtatcgaagcttatgtttccaccgcctctaacccaatcaccgatgcctgtgctttgaagggtattgatttgatcaatgaaagcttagtcgctgcatacaaagacggtaaagacaagaaggccagaactgacatgtgttacgctgaatacttggcaggtatggctttcaacaatgcttctctaggttatgttcatgcccttgctcatcaacttggtggtttctaccacttgcctcatggtgtttgtaacgctgtcttgttgcctcatgttcaagaggccaacatgcaatgtccaaaggccaagaagagattaggtgaaattgctttgcatttcggtgcttctcaagaagatccagaagaaaccatcaaggctttgcacgttttaaacagaaccatgaacattccaagaaacttgaaagaattaggtgttaaaaccgaagattttgaaattttggctgaacacgccatgcatgatgcctgccatttgactaacccagttcaattcaccaaagaacaagtggttgccattatcaagaaagcctatgaatattaa

SEQ ID NO: 48 - protein, 351 aa, Saccharomyces cerevisae
  misc_feature: Adh5 Amino acid sequence
MPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSK

SEQ ID NO: 49 - DNA, 1056 bp, Artificial Sequence
  Synthetic: adh5 Nucleotide sequence
atgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaatga

SEQ ID NO: 50 - protein, 361 aa, Saccharomyces cerevisae
  misc_feature: Adh7 Amino acid sequence
MLYPEKFQGIGISNAKDWKHPKLVSFDPKPFGDHDVDVEIEACGICGSDFHIAVGNWGPVPENQILGHEIIGRVVKVGSKCHTGVKIGDRVGVGAQALACFECERCKSDNEQYCTNDHVLTMWTPYKDGYISQGGFASHVRLHEHFAIQIPENIPSPLAAPLLCGGITVFSPLLRNGCGPGKRVGIVGIGGIGHMGILLAKAMGAEVYAFSRGHSKREDSMKLGADHYIAMLEDKGWTEQYSNALDLLVVCSSSLSKVNFDSIVKIMKIGGSIVSIAAPEVNEKLVLKPLGLMGVSISSSAIGSRKEIEQLLKLVSEKNVKIWVEKLPISEEGVSHAFTRMESGDVKYRFTLVDYDKKFHK

SEQ ID NO: 51 - DNA, 1086 bp, Artificial Sequence
  Synthetic: adh7 Nucleotide sequence
atgctttacccagaaaaatttcagggcatcggtatttccaacgcaaaggattggaagcatcctaaattagtgagttttgacccaaaaccctttggcgatcatgacgttgatgttgaaattgaagcctgtggtatctgcggatctgattttcatatagccgttggtaattggggtccagtcccagaaaatcaaatccttggacatgaaataattggccgcgtggtgaaggttggatccaagtgccacactggggtaaaaatcggtgaccgtgttggtgttggtgcccaagccttggcgtgttttgagtgtgaacgttgcaaaagtgacaacgagcaatactgtaccaatgaccacgttttgactatgtggactccttacaaggacggctacatttcacaaggaggctttgcctcccacgtgaggcttcatgaacactttgctattcaaataccagaaaatattccaagtccgctagccgctccattattgtgtggtggtattacagttttctctccactactaagaaatggctgtggtccaggtaagagggtaggtattgttggcatcggtggtattgggcatatggggattctgttggctaaagctatgggagccgaggtttatgcgttttcgcgaggccactccaagcgggaggattctatgaaactcggtgctgatcactatattgctatgttggaggataaaggctggacagaacaatactctaacgctttggaccttcttgtcgtttgctcatcatctttgtcgaaagttaattttgacagtatcgttaagattatgaagattggaggctccatcgtttcaattgctgctcctgaagttaatgaaaagcttgttttaaaaccgttgggcctaatgggagtatcaatctcaagcagtgctatcggatctaggaaggaaatcgaacaactattgaaattagtttccgaaaagaatgtcaaaatatgggtggaaaaacttccgatcagcgaagaaggcgtcagccatgcctttacaaggatggaaagcggagacgtcaaatacagatttactttggtcgattatgataagaaattccataaatag

SEQ ID NO: 52 - protein, 386 aa, Saccharomyces cerevisae
  misc_feature: SFA1 Amino acid sequence
MSAATVGKPIKCIAAVAYDAKKPLSVEEITVDAPKAHEVRIKIEYTAVCHTDAYTLSGSDPEGLFPCVLGHEGAGIVESVGDDVITVKPGDHVIALYTAECGKCKFCTSGKTNLCGAVRATQGKGVMPDGTTRFHNAKGEDIYHFMGCSTFSEYTVVADVSVVAIDPKAPLDAACLLGCGVTTGFGAALKTANVQKGDTVAVFGCGTVGLSVIQGAKLRGASKIIAIDINNKKKQYCSQFGATDFVNPKEDLAKDQTIVEKLIEMTDGGLDFTFDCTGNTKIMRDALEACHKGWGQSIIIGVAAAGEEISTRPFQLVTGRVWKGSAFGGIKGRSEMGGLIKDYQKGALKVEEFITHRRPFKEINQAFEDLHNGDCLRTVLKSDEIK

SEQ ID NO: 53 - DNA, 1161 bp, Artificial Sequence
  Synthetic: sfa1 Nucleotide sequence
atgtccgccgctactgttggtaaacctattaagtgcattgctgctgttgcgtatgatgcgaagaaaccattaagtgttgaagaaatcacggtagacgccccaaaagcgcacgaagtacgtatcaaaattgaatatactgctgtatgccacactgatgcgtacactttatcaggctctgatccagaaggacttttcccttgcgttctgggccacgaaggagccggtatcgtagaatctgtaggcgatgatgtcataacagttaagcctggtgatcatgttattgctttgtacactgctgagtgtggcaaatgtaagttctgtacttccggtaaaaccaacttatgtggtgctgttagagctactcaagggaaaggtgtaatgcctgatgggaccacaagatttcataatgcgaaaggtgaagatatataccatttcatgggttgctctactttttccgaatatactgtggtggcagatgtctctgtggttgccatcgatccaaaagctcccttggatgctgcctgtttactgggttgtggtgttactactggttttggggcggctcttaagacagctaatgtgcaaaaaggcgataccgttgcagtatttggctgcgggactgtaggactctccgttatccaaggtgcaaagttaaggggcgcttccaagatcattgccattgacattaacaataagaaaaaacaatattgttctcaatttggtgccacggattttgttaatcccaaggaagatttggccaaagatcaaactatcgttgaaaagttaattgaaatgactgatgggggtctggattttacttttgactgtactggtaataccaaaattatgagagatgctttggaagcctgtcataaaggttggggtcaatctattatcattggtgtggctgccgctggtgaagaaatttctacaaggccgttccagctggtcactggtagagtgtggaaaggctctgcttttggtggcatcaaaggtagatctgaaatgggcggtttaattaaagactatcaaaaaggtgccttaaaagtcgaagaatttatcactcacaggagaccattcaaagaaatcaatcaagcctttgaagatttgcataacggtgattgcttaagaaccgtcttgaagtctgatgaaataaaatag

SEQ ID NO: 54 - protein, 491 aa, E. coli
  misc_feature: IlvC amino acid sequence from E. coli Nissle
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVVNLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNAQLRDVNEAIRSHAIEQVGKKLRGYMTDMKRIAVAG

SEQ ID NO: 55 - DNA, 1476 bp, E. coli
  misc_feature: ilvC gene from E. coli Nissle Nucleotide sequence
atggctaactacttcaatacactgaatctgcgccagcagttggcacagctgggcaaatgtcgctttatggggcgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgctgagaagcgcgcatcctggcgtaaagcaaccgaaaatggttttaaagtgggtacttacgaagaactgatcccgcaggcggatctggtggttaacctgacgccggacaagcagcactctgatgtagtgcgcaccgtacagccactgatgaaagacggcgcggcgctgggctactctcatggtttcaatatcgtagaagtgggtgagcagatccgtaaagacatcaccgtcgtaatggttgcgccgaaatgccctggcaccgaagtacgtgaagagtacaaacgtggattcggcgtaccgacgctgattgccgttcacccggaaaacgatccgaaaggcgaaggcatggcgatcgctaaagcatgggcggctgcaaccggcggtcaccgtgcgggcgttctggaatcctctttcgttgcggaagtgaaatctgacctgatgggcgagcaaaccatcctgtgcggtatgttgcaagctggttctctgctgtgcttcgacaagctggtggaagaaggcaccgatccggcatacgcagaaaaactgattcagttcggttgggaaaccatcaccgaagcgctgaaacagggcggcatcaccctgatgatggaccgtctttctaacccggcgaaactgcgtgcttacgcgctttctgagcaactgaaagagatcatggcgccgctgttccagaaacatatggacgacatcatctccggcgaattctcctccggcatgatggctgactgggccaacgacgataagaaactgctgacctggcgtgaagagactggcaaaaccgcattcgaaaccgcgccgcagtatgaaggcaaaatcggtgaacaggagtacttcgataaaggcgtactgatgatcgcgatggtaaaagcaggcgttgagttggcgtttgaaaccatggttgattccggcatcatcgaagaatctgcttactatgaatcactgcacgaactgccgctgattgccaacaccatcgcccgtaagcgtctgtacgaaatgaacgtggttatctccgatactgccgagtacggtaactatctgttctcttacgcttgtgtgccactgctgaaaccgtttatggcagagctgcaaccgggcgacctgggtaaagctattccggaaggtgcggtagataacgcgcagctgcgtgatgtaaatgaagcgattcgcagccatgcgattgagcaggtaggtaagaaactgcgcggctatatgacggatatgaaacgtattgctgttgcgggttaa

SEQ ID NO: 56 - protein, 1416 aa, Proteus vulgaris
  misc_feature: L-amino acid deaminase L-AAD
ATGGCCATCAGTCGTCGCAAATTCATTATCGGTGGAACGGTCGTCGCCGTTGCCGCCGGTGCGGGGATTTTGACCCCGATGCTGACGCGCGAAGGGCGCTTTGTGCCGGGCACTCCACGCCACGGTTTCGTTGAAGGGACCGAGGGGGCTTTACCCAAACAAGCGGACGTGGTGGTCGTAGGCGCTGGAATTCTTGGTATTATGACGGCCATTAATTTGGTTGAGCGTGGGCTGTCAGTGGTAATTGTGGAGAAGGGCAATATCGCGGGAGAACAAAGCTCTCGCTTCTACGGACAGGCAATTAGCTATAAGATGCCAGATGAGACATTTTTGCTGCACCATCTTGGGAAGCACCGCTGGCGTGAGATGAATGCGAAAGTAGGGATTGATACAACGTACCGTACTCAAGGACGCGTGGAAGTACCGCTTGACGAGGAAGATTTGGTAAACGTCCGCAAATGGATTGACGAACGTTCAAAAAATGTTGGATCTGACATTCCTTTTAAGACCCGCATTATCGAGGGGGCAGAATTAAATCAGCGTCTGCGCGGCGCCACAACAGATTGGAAGATCGCTGGCTTCGAGGAGGACAGCGGGTCATTCGATCCCGAGGTAGCGACCTTTGTAATGGCAGAGTACGCGAAGAAGATGGGTGTTCGTATCTATACGCAATGCGCGGCCCGCGGTCTGGAAACCCAGGCCGGTGTCATTTCAGATGTTGTCACGGAAAAAGGTGCGATTAAGACCTCCCAAGTGGTAGTGGCTGGTGGGGTGTGGAGTCGTCTGTTCATGCAGAATTTAAACGTCGACGTCCCAACCCTTCCTGCGTATCAGTCACAGCAGTTGATTAGTGGTTCCCCTACCGCACCGGGGGGGAACGTCGCATTACCTGGTGGAATCTTCTTCCGCGAACAGGCGGACGGGACATACGCGACTTCTCCTCGTGTGATTGTTGCCCCAGTTGTGAAGGAGAGCTTCACTTATGGTTACAAGTACTTACCATTATTAGCATTGCCTGATTTCCCTGTTCACATTAGCCTGAATGAACAGTTAATTAATTCGTTTATGCAAAGTACCCACTGGAACTTAGACGAAGTGTCGCCGTTCGAACAATTTCGCAACATGACAGCATTACCTGACTTGCCCGAACTTAACGCCAGCTTAGAAAAGTTAAAGGCAGAGTTCCCTGCTTTCAAAGAATCCAAGTTGATCGACCAGTGGTCTGGAGCAATGGCAATTGCGCCCGACGAAAATCCAATCATTTCCGAGGTGAAGGAGTACCCAGGTCTGGTAATTAACACGGCGACAGGTTGGGGCATGACTGAAAGTCCAGTGTCTGCTGAACTTACCGCCGATCTTCTGCTGGGGAAGAAGCCGGTGTTAGATCCTAAGCCATTCTCACTTTATCGCTTTTGA

SEQ ID NO: 57 - protein, 471 aa, Artificial Sequence; identical to SEQ ID NO: 23
  Synthetic: amino acid sequence

SEQ ID NO: 58 - DNA, 1101 bp, Bacillus cereus
  misc_feature: Leucine dehydrogenase leuDH
atgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctga

SEQ ID NO: 59 - protein, 366 aa, Artificial Sequence
  Synthetic: amino acid sequence
MTLEIFEYLEKYDYEQVVFCQDKESGLKAIIAIHDTTLGPALGGTRMWTYDSEEAAIEDALRLAKGMTYKNAAAGLNLGGAKTVIIGDPRKDKSEAMFRALGRYIQGLNGRYITAEDVGTTVDDMDIIHEETDFVTGISPSFGSSGNPSPVTAYGVYRGMKAAAKEAFGTDNLEGKVIAVQGVGNVAYHLCKHLHAEGAKLIVTDINKEAVQRAVEEFGASAVEPNEIYGVECDIYAPCALGATVNDETIPQLKAKVIAGSANNQLKEDRHGDIIHEMGIVYAPDYVINAGGVINVADELYGYNRERALKRVESIYDTIAKVIEISKRDGIATYVAADRLAEERIASLKNSRSTYLRNGHDIISRR

SEQ ID NO: 60 - DNA, 1164 bp, E. coli
  misc_feature: Alcohol dehydrogenase YqhD
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtactggaatttggcggtattgaaccaaacccggcttatgaaacgctgatgaacgccgtgaaactggttcgcgaacagaaagtgacgttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagcccgtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctgatcgaagatggtccgaaagccctgaaagagccagaaaactacgatgtgcgcgccaacgtcatgtgggcggcgactcaggcgctgaacggtttgatcggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttcagacgatgagcgtattgacgccgcgattgccgcaacccgcaatttctttgagcaattaggcgtgctgacccacctctccgactacggtctggacggcagctccatcccggctttgctgaaaaaactggaagagcacggcatgacccaactgggcgaaaatcatgacattacgctggatgtcagccgccgtatatacgaagccgcccgctaa

SEQ ID NO: 61 - protein, 387 aa, Artificial Sequence
  Synthetic: amino acid sequence
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVLTHLSDYGLDGSSIPALLKKLEEHGMTQLGENHDITLDVSRRIYEAAR

SEQ ID NO: 62 - DNA, 1500 bp, E. coli
  misc_feature: Aldehyde dehydrogenase PadA
atgacagagccgcatgtagcagtattaagccaggtccaacagtttctcgatcgtcaacacggtctttatattgatggtcgtcctggccccgcacaaagtgaaaaacggttggcgatctttgatccggccaccgggcaagaaattgcgtctactgctgatgccaacgaagcggatgtagataacgcagtcatgtctgcctggcgggcctttgtctcgcgtcgctgggccgggcgattacccgcagagcgtgaacgtattctgctacgttttgctgatctggtggagcagcacagtgaggagctggcgcaactggaaaccctggagcaaggcaagtcaattgccatttcccgtgcttttgaagtgggctgtacgctgaactggatgcgttataccgccgggttaacgaccaaaatcgcgggtaaaacgctggacttgtcgattcccttaccccagggggcgcgttatcaggcctggacgcgtaaagagccggttggcgtagtggcgggaattgtgccatggaactttccgttgatgattggtatgtggaaggtgatgccagcactggcagcaggctgttcaatcgtgattaagccttcggaaaccacgccactgacgatgttgcgcgtggcggaactggccagcgaggctggtatccctgatggcgtttttaatgtcgtcaccgggtcaggtgctgtatgcggcgcggccctgacgtcacatcctcatgttgcgaaaatcagttttaccggttcaaccgcgacgggaaaaggtattgccagaactgctgctgatcacttaacgcgtgtaacgctggaactgggcggtaaaaacccggcaattgtattaaaagatgctgatccgcaatgggttattgaaggcttgatgaccggaagcttcctgaatcaagggcaagtatgcgccgccagttcgcgaatttatattgaagcgccgttgtttgacacgctggttagtggatttgagcaggcggtaaaatcgttgcaagtgggaccggggatgtcacctgttgcacagattaaccctttggtttctcgtgcgcactgcgacaaagtgtgttcattcctcgacgatgcgcaggcacagcaagcagagctgattcgcgggtcgaatggaccagccggagaggggtattatgttgcgccaacgctggtggtaaatcccgatgctaaattgcgcttaactcgtgaagaggtgtttggtccggtggtaaacctggtgcgagtagcggatggagaagaggcgttacaactggcaaacgacacggaatatggcttaactgccagtgtctggacgcaaaatctctcccaggctctggaatatagcgatcgcttacaggcagggacggtgtgggtaaacagccataccttaattgacgctaacttaccgtttggtgggatgaagcagtcaggaacgggccgtgattttggccccgactggctggacggttggtgtgaaactaagtcggtgtgtgtacggtattaa

SEQ ID NO: 63 - protein, 499 aa, Artificial Sequence
  Synthetic: amino acid sequence
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 64 - DNA, 1320 bp, E. coli
  misc_feature: BCAA transporter BrnQ
atgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaa

SEQ ID NO: 65 - protein, 439 aa, Artificial Sequence
  Synthetic: amino acid sequence
MTHQLRSRDIIALGFMTFALFVGAGNIIFPPMVGLQAGEHVWTAAFGFLITAVGLPVLTVVALAKVGGGVDSLSTPIGKVAGVLLATVCYLAVGPLFATPRTATVSFEVGIAPLTGDSALPLFIYSLVYFAIVILVSLYPGKLLDTVGNFLAPLKIIALVILSVAAIIWPAGSISTATEAYQNAAFSNGFVNGYLTMDTLGAMVFGIVIVNAARSRGVTEARLLTRYTVWAGLMAGVGLTLLYLALFRLGSDSASLVDQSANGAAILHAYVQHTFGGGGSFLLAALIFIACLVTAVGLTCACAEFFAQYVPLSYRTLVFILGGFSMVVSNLGLSQLIQISVPVLTAIYPPCIALVVLSFTRSWWHNSSRVIAPPMFISLLFGILDGIKASAFSDILPSWAQRLPLAEQGLAWLMPTVVMVVLAIIWDRAAGRQVTSSAH

SEQ ID NO: 66 - protein, 541 aa, Streptomyces lividans
  misc_feature: Isovaleryl-CoA synthetase LbuL
MTAPAPQPSYAHGTSTTPLLGDTVGANLGRAIAAHPDREALVDVPSGRRWTYAEFGAAVDELARGLLAKGVTRGDRVGIWAVNCPEWVLVQYATARIGVIMVNVNPAYRAHELEYVLQQSGISLLVASLAHKSSDYRAIVEQVRGRCPALRETVYIGDPSWDALTAGAAAVEQDRVDALAAELSCDDPVNIQYTSGTTGFPKGATLSHHNILNNGYWVGRTVGYTEQDRVCLPVPFYHCFGMVMGNLGATSHGACIVIPAPSFEPAATLEAVQRERCTSLYGVPTMFIAELNLPDFASYDLTSLRTGIMAGSPCPVEVMKRVVAEMHMEQVSICYGMTETSPVSLQTRMDDDLEHRTGTVGRVLPHIEVKVVDPVTGVTLPRGEAGELRTRGYSVMLGYWEEPGKTAEAIDPGRWMHTGDLAVMREDGYVEIVGRIKDMIIRGGENIYPREVEEFLYAHPKIADVQVVGVPHERYGEEVLACVVVRDAADPLTLEELRAYCAGQLAHYKVPSRLQLLDSFPMTVSGKVRKVELRERYGTRP

SEQ ID NO: 67 - DNA, 1626 bp, Artificial Sequence
  Synthetic: codon-optimized Nucleotide sequence
atgactgcaccagcacctcaaccctcttatgcacatggcacttctaccactccgcttcttggtgatacggtgggggcaaacctgggtcgtgccatcgcggctcatcccgatcgtgaggcactggtcgatgtacccagcggacgccgttggacttacgcagagtttggcgcggccgtagatgaattagcacgcggcctgttagccaaaggggtaactcgcggtgaccgtgtgggtatttgggctgtgaactgtcccgaatgggttttggtgcaatacgctacagcccgtattggggtaatcatggttaatgtaaatcccgcttatcgcgcccacgagcttgaatatgtactgcaacagagtggcatttccttattagtggcttcacttgcacacaaaagttcagattaccgcgcaattgtggagcaagtgcgcggccgctgtcccgccttacgtgaaactgtgtacatcggtgatccatcatgggatgccttgactgcaggcgcagcggctgtcgaacaagatcgtgttgacgctctggcggcggagctttcatgtgacgaccctgtcaacattcagtacactagcggtacgactggttttccgaaaggagcaacattatctcaccataacatcttgaacaacggttattgggtagggcgcacagtcggctacactgagcaagaccgtgtctgcttaccagtcccgttctatcattgctttgggatggtgatgggaaatcttggagccacatcccatggggcctgtattgtgatcccggccccctccttcgagcctgccgcgactttagaagctgttcagcgcgaacgttgtacaagcctgtacggcgttcccacaatgtttattgcggagcttaacctgccggactttgcctcatacgatttgacgagcctgcgcactggcatcatggcagggtcgccctgcccagtagaagtcatgaagcgtgtcgttgctgagatgcatatggagcaggtctcgatttgttatggtatgacggagaccagtcccgtgagtcttcaaactcgcatggacgacgacttagaacaccgtacaggtacggtcggtcgtgtacttccgcacattgaagtcaaagtagtggaccccgtgacaggtgtaacccttccccgcggggaggcaggggagcttcgcactcgtggatacagcgtaatgctgggttattgggaggaacctggcaagacggctgaggctatcgatccgggtcgttggatgcacacaggcgatcttgcggtgatgcgtgaagatgggtatgttgagattgttgggcgcatcaaggacatgattattcgcggcggtgaaaacatttatcctcgcgaggttgaagaatttttatatgcacacccaaagatcgcagacgtacaggtagtcggcgtgccacatgagcgttatggagaagaggtactggcgtgcgttgtcgttcgcgacgcggccgacccactgaccctggaagaattacgcgcctactgtgcaggccagcttgctcattataaagtcccttcgcgtttacaacttttggattcgttccctatgaccgtgtcaggaaaggtacgtaaggttgagttacgtgagcgctacgggacacgcccgtga

SEQ ID NO: 68 - protein, 387 aa, Pseudomonas aeruginosa
  misc_feature: liuA
MTYPSLNFALGETIDMLRDQVRGFVAAELQPRAAQIDQDNQFPMDMWRKFGEMGLLGITVDEEYGGSALGYLAHAVVMEEISRASASVALSYGAHSNLCVNQIKRNGNAEQKARYLPALVSGEHIGALAMSEPNAGSDVVSMKLRADRVGDRFVLNGSKMWITNGPDAHTYVIYAKTDADKGAHGITAFIVERDWKGFSRGPKLDKLGMRGSNTCELIFQDVEVPEENVLGAVNGGVKVLMSGLDYERVVLSGGPVGIMQACMDVVVPYIHDRRQFGQSIGEFQLVQGKVADMYTALNASRAYLYAVAAACDRGETTRKDAAGVILYSAERATQMALDAIQILGGNGYINEFPTGRLLRDAKLYEIGAGTSEIRRMLIGRELFNETR

SEQ ID NO: 69 - protein, 535 aa, Pseudomonas aeruginosa
  misc_feature: LiuB
MAILHTQINPRSAEFAANAATMLEQVNALRTLLGRIHEGGGSAAQARHSARGKLLVRERINRLLDPGSPFLELSALAAHEVYGEEVAAAGIVAGIGRVEGVECMIVGNDATVKGGTYYPLTVKKHLRAQAIALENRLPCIYLVDSGGANLPRQDEVFPDREHFGRIFFNQANMSARGIPQIAVVMGSCTAGGAYVPAMSDETVMVREQATIFLAGPPLVKAATGEVVSAEELGGADVHCKVSGVADHYAEDDDHALAIARRCVANLNWRKQGQLQCRAPRAPLYPAEELYGVIPADSKQPYDVREVIARLVDGSEFDEFKALFGTTLVCGFAHLHGYPIAILANNGILFAEAAQKGAHFIELACQRGIPLLFLQNITGFMVGQKYEAGGIAKHGAKLVTAVACARVPKFTVLIGGSFGAGNYGMCGRAYDPRFLWMWPNARIGVMGGEQAAGVLAQVKREQAERAGQQLGVEEEAKIKAPILEQYEHQGHPYYSSARLWDDGVIDPAQTREVLALALSAALNAPIEPTAFGVFRM

SEQ ID NO: 70 - protein, 265 aa, Pseudomonas aeruginosa
  misc_feature: LiuC
MSEFQTIQLEIDPRGVATLWLDRAEKNNAFNAVVIDELLQAIDRVGSDPQVRLLVLRGRGRHFCGGADLAWMQQSVDLDYQGNLADAQRIAELMTHLYNLPKPTLAVVQGAVFGGGVGLVSCCDMAIGSDDATFCLSEVRIGLIPATIAPFVVKAIGQRAARRYSLTAERFDGRRASELGLLSESCPAAELESQAEAWIANLLQNSPRALVACKALYHEVEAAELSPALRRYTEAAIARIRISPEGQEGLRAFLEKRTPAWRNDA

SEQ ID NO: 71 - protein, 655 aa, Pseudomonas aeruginosa
  misc_feature: LiuD
MNPDYRSIQRLLVANRGEIACRVMRSARALGIGSVAVHSDIDRHARHVAEADIAVDLGGAKPADSYLRGDRIIAAALASGAQAIHPGYGFLSENADFARACEEAGLLFLGPPAAAIDAMGSKSAAKALMEEAGVPLVPGYHGEAQDLETFRREAGRIGYPVLLKAAAGGGGKGMKVVEREAELAEALSSAQREAKAAFGDARMLVEKYLLKPRHVEIQVFADRHGHCLYLNERDCSIQRRHQKVVEEAPAPGLGAELRRAMGEAAVRAAQAIGYVGAGTVEFLLDERGQFFFMEMNTRLQVEHPVTEAITGLDLVAWQIRVARGEALPLTQEQVPLNGHAIEVRLYAEDPEGDFLPASGRLMLYREAAAGPGRRVDSGVREGDEVSPFYDPMLAKLIAWGETREEARQRLLAMLAETSVGGLRTNLAFLRRILGHPAFAAAELDTGFIARHQDDLLPAPQALPEHFWQAAAEAWLQSEPGHRRDDDPHSPWSRNDGWRSALARESDLMLRCRDERRCVRLRHASPSQYRLDGDDLVSRVDGVTRRSAALRRGRQLFLEWEGELLAIEAVDPIAEAEAAHAHQGGLSAPMNGSIVRVLVEPGQTVEAGATLVVLEAMKMEHSIRAPHAGVVKALYCSEGELVEEGTPLVELDENQA

SEQ ID NO: 72 - protein, 300 aa, Pseudomonas aeruginosa
  misc_feature: LiuE
MNLPKKVRLVEVGPRDGLQNEKQPIEVADKIRLVDDLSAAGLDYIEVGSFVSPKWVPQMAGSAEVFAGIRQRPGVTYAALAPNLKGFEAALESGVKEVAVFAAASEAFSQRNINCSIKDSLERFVPVLEAARQHQVRVRGYISCVLGCPYDGDVDPRQVAWVARELQQMGCYEVSLGDTIGVGTAGATRRLIEAVASEVPRERLAGHFHDTYGQALANIYASLLEGIAVFDSSVAGLGGCPYAKGATGNVASEDVLYLLNGLEIHTGVDMHALVDAGQRICAVLGKSNGSRAAKALLAKA

SEQ ID NO: 73 - DNA, 6595 bp, Pseudomonas aeruginosa
  misc_feature: liuABCDE codon optimized sequence
atgacttacccgtccctgaattttgcgctgggcgaaaccattgacatgttgcgcgaccaagttcgtggcttcgttgcagcggaactgcaacctcgcgcggctcaaattgaccaggataatcagtttccgatggatatgtggcgtaagttcggtgagatggggctcttaggtattacggttgatgaggaatacggaggtagcgcgctcggttacttagcccatgcggtcgtaatggaagaaatttcccgtgcctctgcgagcgtagcgctgtcttatggtgcgcattcaaacctgtgcgttaaccagatcaaacgcaatggtaacgctgaacagaaagcgcgttatctgccggctttggtgtccggcgaacacattggcgccctcgctatgtcggaacctaacgcagggtcggatgtggtgtctatgaaactgcgcgcggatcgcgttggcgatcgtttcgtgctgaatggttccaaaatgtggatcaccaacgggcctgatgcacatacgtatgtgatctacgctaaaaccgacgcagataaaggggcccatggcatcaccgcatttattgttgagcgtgactggaaagggtttagccgtggcccaaaactggataaactcggtatgcgtggttcaaatacatgtgaactgattttccaagacgtcgaagtccccgaagaaaatgtgctgggtgcagtgaatgggggggtcaaagtgttaatgtctggtctcgattatgaacgtgtagtgctgagcggtggtccggttggtattatgcaagcctgtatggacgtggtagtgccgtacattcatgatcgccgccagttcggccagtcgatcggagaatttcagctggtgcagggtaaggttgcggacatgtataccgctctgaatgcttctcgtgcgtacttgtatgctgtcgctgcagcctgcgatcgtggagaaacgactcgcaaagacgctgctggtgtgattctctacagcgcagaacgtgctacccaaatggcacttgacgcgatccagatcttgggaggcaatgggtatatcaatgagttccccacgggccgcctgctgcgcgatgcgaagctgtatgagatcggcgcgggtacgagcgaaatccgccgtatgttaatcggtcgtgaattatttaacgagactcgctgaagcctcgctcttcccggcccttttccgccagggagagggcattccattgcatcgacaggcgcatcgccaggtcgggagcgggcgccaaccgcttccgcccacctcgacacggagccaccgccatggccatccttcacacgcagattaacccgcgttctgctgaattcgcggcgaatgccgcgaccatgctggagcaagttaacgcattgcgtacgctccttggtcgcatccacgaaggtggtggttcggcggctcaggctcgccattcggcacgtggcaaattgttggttcgcgaacgcatcaaccgcctgctggaccccggtagcccgtttttggagttgagcgcgttagcagctcatgaggtgtatggggaagaagtcgcagcagcaggtatcgtggccgggatcgggcgtgtagaaggagtagaatgtatgatcgttggtaatgatgccactgtgaaaggaggtacgtattacccgctgaccgtgaagaagcatctgcgcgcccaagcaatcgcattagaaaatcgtttgccgtgtatctatctggtcgattcgggtggcgccaatctgcctcgccaggacgaggtctttccggatcgcgagcatttcggccgcatctttttcaaccaagccaatatgagcgcccgcggtatcccgcagattgcggtggtaatgggctcatgtactgcgggtggcgcctatgtcccggccatgtccgatgaaactgtgatggtccgtgagcaggcgacgatcttcctggctggaccgcctctcgtgaaagcggccacgggtgaagtggtttcagcagaggaattgggtggcgccgacgtgcattgtaaagtgtcaggcgtggcggaccactatgccgaagatgatgaccatgcattggcgattgcgcgtcgctgtgttgcgaatttaaattggcgcaaacagggtcagcttcagtgccgtgcgccgcgtgctccgctgtatccggcggaagaactgtatggtgtgattccggcggatagcaaacagccgtatgatgtgcgcgaggtcattgcacgcctggttgatggatctgaatttgatgaattcaaggcgctgttcggaaccaccctggtgtgcggctttgcacacctgcatggctacccaattgccattctcgcaaataatggcattctgttcgcggaggcggcccagaaaggggcccatttcattgaactggcctgccaacgcggtattccattactgttcctgcaaaatatcaccggcttcatggttggtcagaagtatgaagctggcggtattgccaagcatggcgcgaaactggtcaccgcggtcgcctgcgcccgcgtgccgaaatttacagtgctgattggcggaagtttcggggcagggaactacggaatgtgtggtcgcgcgtacgatccgcgcttcctctggatgtggccgaatgcacgcattggcgtgatgggcggcgagcaggctgccggcgtcctggcacaggtcaaacgtgagcaagcggaacgcgctggccaacagctgggggtggaggaagaagcgaaaattaaagcgccgatccttgaacagtatgaacatcagggccatccgtactattcgtcagcacgtttgtgggacgatggcgtcattgatcctgcccagacacgcgaagtccttgcgctggcgctgagtgcggcgcttaacgctccgatcgaaccaactgcattcggtgtatttcgcatgtgacgagtagaccagcatgagcgaatttcagacgatccagctggaaattgatccacgtggagtggcaaccctgtggctggaccgtgctgaaaaaaataacgcatttaacgccgtcgtgatcgatgaactgctgcaggcgatcgaccgcgtaggcagcgacccccaggtccgtttgctggtcttgcgtgggcgtggccgtcatttctgtggcggcgccgacctggcgtggatgcagcagtctgttgacctggattatcagggtaaccttgctgacgcccagcgcatcgcagagctcatgacccacttgtataatctgcccaaacctactttagcggtagttcaaggcgcagttttcggcggcggggtcggtttggtgagctgctgcgacatggcaattggtagtgatgacgccactttttgcttgtcagaggtacgcattgggctgattccagcaaccatcgccccgttcgtggtgaaagctattggtcaacgcgcagcgcgccgttattcactgactgctgaacgttttgatgggcgccgcgcgtccgaactgggactgcttagcgagtcttgcccggccgcagaactggaatcccaagcggaagcatggatcgcgaatcttctccagaactctccacgtgcactcgtggcatgtaaagcgctgtatcacgaggtagaagcggctgaactgtcccctgcactgcgtcgctatacggaagccgcaattgcacgtatccgtatttcaccagaaggtcaagaaggcttgcgtgcctttttagaaaaacgcacaccggcgtggagaaacgacgcatgaacccggactaccgttcaattcagcgtctcttagtagctaaccgtggcgagattgcctgtcgcgtaatgcgttcggcccgcgcgttaggtattggatcagttgcagttcattcggatatcgaccgccacgcacgtcacgtggctgaagctgatattgcggttgacctgggcggcgccaaaccggcagattcgtatctgcgtggcgaccgtatcattgcagctgcactggcttcaggagcccaggccattcatccggggtatggctttctgtctgagaatgctgattttgcccgcgcgtgcgaagaagcaggtttactgtttttgggcccaccggctgcggcaattgatgctatggggtctaagtcagcggcgaaagctttgatggaagaggcgggagtccccctggttccaggttaccacggtgaagcgcaggacttggaaacctttcgtcgcgaggccggacgcatcggctatcccgtgctcttaaaggccgcggccggtggcggcggaaaagggatgaaagtcgtggaacgcgaggccgagctcgcagaagcgctgtccagcgcccaacgcgaagccaaagcggcctttggcgatgcgcgcatgctggtggagaagtatttgttaaaaccgcgtcacgtcgaaattcaggtctttgcagatcgtcatggtcactgtttatacctcaacgaacgtgactgttcgatccaacgtcgccatcaaaaagttgtagaagaagcgccggctcccggtttgggcgcggaactgcgtcgtgccatgggcgaagcggccgttcgcgcagcgcaagcgatcggctatgtgggggcgggcactgtagagtttctcctggacgagcgcggtcaattcttttttatggaaatgaacactcgcctgcaggttgaacaccctgtaactgaggccatcactggtctcgatttagtcgcgtggcagatccgtgtggcgcgtggtgaagcccttccgttgactcaagaacaagtaccgctgaacgggcacgcgatcgaagtccgcctgtacgcggaagaccctgaaggggattttcttccggcaagtggacgcctgatgctgtatcgtgaagccgctgcaggtccgggccgccgcgtggattcgggagtccgtgagggcgacgaagtcagccccttctacgatccgatgctggcaaaattgatcgcatggggggaaacccgtgaggaagctcgccaacgcctgctcgccatgttggccgagacctcggtcgggggcttgcgtacgaacctggcttttttacgtcgtatcttaggccatcccgcttttgccgccgctgaactggataccgggttcattgctcgtcatcaagatgacctgctgccagcaccccaggctctgccagaacacttctggcaagcagcagcagaagcttggctgcaaagcgaacctggtcatcgtcgcgatgacgatccgcattccccttggagccgtaacgatggttggcgctctgctttggcacgcgaatctgatctgatgctgcgctgtcgcgatgaacgccgttgtgtgcgtctgcgccatgcttccccatctcaatatcgtcttgacggtgatgatctggtatcccgtgttgatggcgttacccgccgctccgcagcgttgcgtcgcggccgccagctgttcttagaatgggaaggtgaactgttagcgatcgaagctgttgatccgattgcagaagccgaagcggcgcatgcccatcaaggcggtttgagcgcgccaatgaacgggtctattgtacgcgttctggttgagccggggcaaaccgtagaggcgggtgcgactcttgtggttttagaagcaatgaaaatggagcacagtatccgtgcgccacatgccggcgttgttaaagcgctgtactgttcagaaggagaattagttgaagagggcactcctctggttgaactggacgaaaaccaggcctgacagccaagacgaggaacagcatgaacctgccgaagaaagttcgtctggttgaagttggtccgcgcgatggacttcagaacgaaaaacagccgatcgaagtggctgacaaaattcgccttgttgatgacttgtcggcagccggcttagattatattgaagtgggcagtttcgtctcaccgaaatgggttccgcagatggccgggagcgccgaagtgtttgctggcattcgtcaacgccctggcgtgacctacgcggcactcgccccgaatttgaaaggcttcgaagcagctctggaatcgggtgtaaaagaagttgccgtgttcgcagcagcctccgaagcattctcccaacgcaacatcaactgctcgattaaagactcccttgagcgcttcgtcccggttctggaagcggctcgccaacatcaggtacgcgtccgcggatatatttcctgcgtattgggttgcccgtatgatggcgacgtagatccgcgccaggtcgcatgggtcgcacgtgaactccagcagatgggctgctatgaggtcagtctcggcgatacaatcggtgtgggtaccgcgggcgcgacccgccgtttaattgaggcggtggcatctgaggttccccgcgaacgccttgcaggccactttcatgatacatatggacaggcgctggctaacatctatgcttctttgctggagggcattgctgtcttcgacagttccgtagctggcctcggtggctgcccatatgcaaaaggcgctaccggcaacgtcgcgagtgaggatgtgctgtatcttttaaatggtcttgaaattcataccggtgtggacatgcatgccctggtagacgcgggacagcgcatctgtgcggtgctcggaaagtcgaatggctcccgtgctgcgaaggccctgctggccaaagcttaatga

SEQ ID NO: 74 - skipped

SEQ ID NO: 75 - DNA, 3443 bp, Artificial Sequence
  Synthetic: Tet-kivD-leuDH construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataagaaggagatatacatatgttcgacatgatggatgcagcccgcctggaaggcctgcacctcgcccaggatccagcgacgggcctgaaagcgatcatcgcgatccattccactcgcctcggcccggccttaggcggctgtcgttacctcccatatccgaatgatgaagcggccatcggcgatgccattcgcctggcgcagggcatgtcctacaaagctgcacttgcgggtctggaacaaggtggtggcaaggcggtgatcattcgcccaccccacttggataatcgcggtgccttgtttgaagcgtttggacgctttattgaaagcctgggtggccgttatatcaccgccgttgactcaggaacaagtagtgccgatatggattgcatcgcccaacagacgcgccatgtgacttcaacgacacaagccggcgacccatctccacatacggctctgggcgtctttgccggcatccgcgcctccgcgcaggctcgcctggggtccgatgacctggaaggcctgcgtgtcgcggttcagggccttggccacgtaggttatgcgttagcggagcagctggcggcggtcggcgcagaactgctggtgtgcgacctggaccccggccgcgtccagttagcggtggagcaactgggggcgcacccactggcccctgaagcattgctctctactccgtgcgacattttagcgccttgtggcctgggcggcgtgctcaccagccagtcggtgtcacagttgcgctgcgcggccgttgcaggcgcagcgaacaatcaactggagcgcccggaagttgcagacgaactggaggcgcgcgggattttatatgcgcccgattacgtgattaactcgggaggactgatttatgtggcgctgaagcatcgcggtgctgatccgcatagcattaccgcccacctcgctcgcatccctgcacgcctgacggaaatctatgcgcatgcgcaggcggatcatcagtcgcctgcgcgcatcgccgatcgtctggcggagcgcattctgtacggcccgcagtga

SEQ ID NO: 76 - DNA, 3467 bp, Artificial Sequence
  Synthetic: Tet-kivD-adh2 construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataataagaaggagatatacatatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataa

SEQ ID NO: 77 - skipped

SEQ ID NO: 78 - DNA, 4530 bp, Artificial Sequence
  Synthetic: Tet-leuDH-kivD-adh2 construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgttcgacatgatggatgcagcccgcctggaaggcctgcacctcgcccaggatccagcgacgggcctgaaagcgatcatcgcgatccattccactcgcctcggcccggccttaggcggctgtcgttacctcccatatccgaatgatgaagcggccatcggcgatgccattcgcctggcgcagggcatgtcctacaaagctgcacttgcgggtctggaacaaggtggtggcaaggcggtgatcattcgcccaccccacttggataatcgcggtgccttgtttgaagcgtttggacgctttattgaaagcctgggtggccgttatatcaccgccgttgactcaggaacaagtagtgccgatatggattgcatcgcccaacagacgcgccatgtgacttcaacgacacaagccggcgacccatctccacatacggctctgggcgtctttgccggcatccgcgcctccgcgcaggctcgcctggggtccgatgacctggaaggcctgcgtgtcgcggttcagggccttggccacgtaggttatgcgttagcggagcagctggcggcggtcggcgcagaactgctggtgtgcgacctggaccccggccgcgtccagttagcggtggagcaactgggggcgcacccactggcccctgaagcattgctctctactccgtgcgacattttagcgccttgtggcctgggcggcgtgctcaccagccagtcggtgtcacagttgcgctgcgcggccgttgcaggcgcagcgaacaatcaactggagcgcccggaagttgcagacgaactggaggcgcgcgggattttatatgcgcccgattacgtgattaactcgggaggactgatttatgtggcgctgaagcatcgcggtgctgatccgcatagcattaccgcccacctcgctcgcatccctgcacgcctgacggaaatctatgcgcatgcgcaggcggatcatcagtcgcctgcgcgcatcgccgatcgtctggcggagcgcattctgtacggcccgcagtgataagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataataagaaggagatatacatatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataatacgcatggcatggatgaa

SEQ ID NO: 79 - DNA, 4434 bp, Artificial Sequence
  Synthetic: Tet-ilvE-kivD-adh2 construct
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaccacgaagaaagctgattacatttggttcaatggggagatggttcgctgggaagacgcgaaggtgcatgtgatgtcgcacgcgctgcactatggcacctcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgttggtgatgttggcatgggcgtaaacccgccagcgggatactcaaccgacgtgattatcgccgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgttggatgtgaatggttacatctctgaaggcgcaggcgaaaacctgtttgaagtgaaagacggcgtgctgttcaccccaccgttcacctcatccgcgctgccgggtattacccgtgatgccatcatcaaactggcaaaagagctgggaattgaagtgcgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataataagaaggagatatacatatgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataataagaaggagatatacatatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataatacgcatggcatggatgaa

SEQ ID NO: 80 - DNA, 117 bp, Artificial Sequence
  Synthetic: FNR-responsive regulatory region Sequence
atccccatcactcttgatggagatcaattccccaagctgctagagcgttaccttgcccttaaacattagcaatgtcgatttatcagagggccgacaggctcccacaggagaaaaccg

SEQ ID NO: 81 - DNA, 108 bp, Artificial Sequence
  Synthetic: FNR-responsive regulatory region Sequence
ctcttgatcgttatcaattcccacgctgtttcagagcgttaccttgcccttaaacattagcaatgtcgatttatcagagggccgacaggctcccacaggagaaaaccg

SEQ ID NO: 82 - DNA, 290 bp, Artificial Sequence; identical to SEQ ID NO: 14
  Synthetic: FNR-responsive regulatory region Sequence: nirB1

SEQ ID NO: 83 - DNA, 433 bp, Artificial Sequence
  Synthetic: FNR-responsive regulatory region Sequence: nirB2
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 84 - DNA, 290 bp, Artificial Sequence
  Synthetic: FNR-responsive regulatory region Sequence: nirB3
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 85 - DNA, 173 bp, Artificial Sequence; identical to SEQ ID NO: 15
  Synthetic: FNR-responsive regulatory region Sequence: ydfZ

SEQ ID NO: 86 - DNA, 305 bp, Artificial Sequence; identical to SEQ ID NO: 16
  Synthetic: FNR-responsive regulatory region Sequence: nirB+RBS

SEQ ID NO: 87 - DNA, 180 bp, Artificial Sequence; identical to SEQ ID NO: 17
  Synthetic: FNR-responsive regulatory region Sequence: ydfZ+RBS

SEQ ID NO: 88 - DNA, 199 bp, Artificial Sequence; identical to SEQ ID NO: 18
  Synthetic: FNR-responsive regulatory region Sequence: fnrS1

SEQ ID NO: 89 - DNA, 207 bp, Artificial Sequence
  Synthetic: FNR-responsive regulatory region Sequence: fnrS2
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 90 - DNA, 390 bp, Artificial Sequence
  Synthetic: FNR-responsive regulatory region Sequence: nirB+crp
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 91 - DNA, 4837 bp, E. coli
  misc_feature: livKHMGF operon
atgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtgatcatcccaccgcccgtaaaatgcgggcgggtttagaaaggttaccttatgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataagatggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaa

SEQ ID NO: 92 - protein, 369 aa, E. coli
  misc_feature: LivK
MKRNAKTIIAGMIALAISHTAMADDIKVAVVGAMSGPIAQWGDMEFNGARQAIKDINAKGGIKGDKLVGVEYDDACDPKQAVAVANKIVNDGIKYVIGHLCSSSTQPASDIYEDEGILMISPGATNPELTQRGYQHIMRTAGLDSSQGPTAAKYILETVKPQRIAIIHDKQQYGEGLARSVQDGLKAANANVVFFDGITAGEKDFSALIARLKKENIDFVYYGGYYPEMGQMLRQARSVGLKTQFMGPEGVGNASLSNIAGDAAEGMLVTMPKRYDQDPANQGIVDALKADKKDPSGPYVWITYAAVQSLATALERTGSDEPLALVKDLKANGANTVIGPLNWDEKGDLKGFDFGVFQWHADGSSTAAK

SEQ ID NO: 93 - DNA, 1110 bp, E. coli
  misc_feature: LivK
atgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtga

SEQ ID NO: 94 - protein, 308 aa, E. coli
  misc_feature: LivH
MSEQFLYFLQQMFNGVTLGSTYALIAIGYTMVYGIIGMINFAHGEVYMIGSYVSFMIIAALMMMGIDTGWLLVAAGFVGAIVIASAYGWSIERVAYRPVRNSKRLIALISAIGMSIFLQNYVSLTEGSRDVALPSLFNGQWVVGHSENFSASITTMQAVIWIVTFLAMLALTIFIRYSRMGRACRACAEDLKMASLLGINTDRVIALTFVIGAAMAAVAGVLLGQFYGVINPYIGFMAGMKAFTAAVLGGIGSIPGAMIGGLILGIAEALSSAYLSTEYKDVVSFALLILVLLVMPTGILGRPEVEKV

SEQ ID NO: 95 - DNA, 927 bp, E. coli
  misc_feature: LivH
atgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatga

SEQ ID NO: 96 - protein, 425 aa, E. coli
  misc_feature: LivM
MKPMHIAMALLSAAMFFVLAGVFMGVQLELDGTKLVVDTASDVRWQWVFIGTAVVFFFQLLRPAFQKGLKSVSGPKFILPAIDGSTVKQKLFLVALLVLAVAWPFMVSRGTVDIATLTMIYIILGLGLNVVVGLSGLLVLGYGGFYAIGAYTFALLNHYYGLGFWTCLPIAGLMAAAAGFLLGFPVLRLRGDYLAIVTLGFGEIVRILLLNNTEITGGPNGISQIPKPTLFGLEFSRTAREGGWDTFSNFFGLKYDPSDRVIFLYLVALLLVVLSLFVINRLLRMPLGRAWEALREDEIACRSLGLSPRRIKLTAFTISAAFAGFAGTLFAARQGFVSPESFTFAESAFVLAIVVLGGMGSQFAVILAAILLVVSRELMRDFNEYSMLMLGGLMVLMMIWRPQGLLPMTRPQLKLKNGAAKGEQA

SEQ ID NO: 97 - DNA, 1278 bp, E. coli
  misc_feature: LivM
atgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatga

SEQ ID NO: 98 - protein, 255 aa, E. coli
  misc_feature: LivG
MSQPLLSVNGLMMRFGGLLAVNNVNLELYPQEIVSLIGPNGAGKTTVFNCLTGFYKPTGGTILLRDQHLEGLPGQQIARMGVVRTFQHVRLFREMTVIENLLVAQHQQLKTGLFSGLLKTPSFRRAQSEALDRAATWLERIGLLEHANRQASNLAYGDQRRLEIARCMVTQPEILMLDEPAAGLNPKETKELDELIAELRNHHNTTILLIEHDMKLVMGISDRIYVVNQGTPLANGTPEQIRNNPDVIRAYLGEA

SEQ ID NO: 99 - skipped

SEQ ID NO: 100 - DNA, 768 bp, E. coli
  misc_feature: LivG
atgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataa

SEQ ID NO: 101 - protein, 237 aa, E. coli [sequence omitted]

SEQ ID NO: 102 - DNA, 714 bp, E. coli
  misc_feature: LivF
atggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaa

SEQ ID NO: 103 - DNA, 305 bp, Artificial Sequence
  Synthetic: Arabinose Promoter region
cagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacctctagaaataattttgtttaactttaagaaggagatatacat

SEQ ID NO: 104 - DNA, 897 bp, Artificial Sequence
  Synthetic: AraC
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcat

SEQ ID NO: 105 - protein, 298 aa, Artificial Sequence
  Synthetic: AraC polypeptide
MQYGQLVSSLNGGSMKSMAEAQNDPLLPGYSFNAHLVAGLTPIEANGYLDFFIDRPLGMKGYILNLTIRGQGVVKNQGREFVCRPGDILLFPPGEIHHYGRHPEAHEWYHQWVYFRPRAYWHEWLNWPSIFANTGFFRPDEAHQPHFSDLFGQIINAGQGEGRYSELLAINLLEQLLLRRMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCE

SEQ ID NO: 106 - DNA, 280 bp, Artificial Sequence
  Synthetic: Region comprising rhamnose inducible promoter
cggtgagcatcacatcaccacaattcagcaaattgtgaacatcatcacgttcatctttccctggttgccaatggcccattttcctgtcagtaacgagaaggtcgcgaatcaggcgctttttagactggtcgtaatgaaattcagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 107 - DNA, 326 bp, Artificial Sequence
  Synthetic: Lac Promoter region
attcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 108 - DNA, 22 bp, Artificial Sequence
  Synthetic: LacO
ggaattgtgagcgctcacaatt

SEQ ID NO: 109 - DNA, 1083 bp, Artificial Sequence
  Synthetic: LacI
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcat

SEQ ID NO: 110 - protein, 360 aa, Artificial Sequence
  Synthetic: LacI polypeptide sequence
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 111 - DNA, 748 bp, Artificial Sequence
  Synthetic: TetR-tet promoter construct
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacat

SEQ ID NO: 112 - DNA, 222 bp, Artificial Sequence
  Synthetic: Region comprising Temperature sensitive promoter
acgttaaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 113 - DNA, 714 bp, Artificial Sequence
  Synthetic: mutant cI857 repressor
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 114 - DNA, 43 bp, Artificial Sequence
  Synthetic: RBS and leader region
ctctagaaataattttgtttaactttaagaaggagatatacat

SEQ ID NO: 115 - protein, 237 aa, Artificial Sequence
  Synthetic: mutant cI857 repressor polypeptide sequence
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 116 - DNA, 225 bp, Artificial Sequence
  Synthetic: PssB promoter
tcacctttcccggattaaacgcttttttgcccggtggcatggtgctaccggcgatcacaaacggttaattatgacacaaattgacctgaatgaatatacagtattggaatgcattacccggagtgttgtgtaacaatgtctggccaggtttgtttcccggaaccgaggtcacaacatagtaaaagcgctattggtaatggtacaatcgcgcgtttacacttattc

SEQ ID NO: 117 - DNA, 207 bp, Artificial Sequence; identical to SEQ ID NO: 89
  Synthetic: FNR promoter with RBS and leader region

SEQ ID NO: 118 - DNA, 14 bp, Artificial Sequence
  Synthetic: FNR binding site
ttgagcgaagtcaa

SEQ ID NO: 119 - DNA, 164 bp, Artificial Sequence
  Synthetic: FNR promoter without RBS and leader region
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaa

SEQ ID NO: 120 - DNA, 43 bp, Artificial Sequence; identical to SEQ ID NO: 114
  Synthetic: RBS and leader region

SEQ ID NO: 121 - DNA, 5169 bp, Artificial Sequence
  Synthetic: LeuDH-kivD-adh2-brnQ construct
atgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgtctattccagaaacgcagaaagccatcatattttatgaatcgaacggaaaacttgagcacaaggacatccccgtcccgaagccaaaacctaatgagttgcttatcaacgttaagtattcgggcgtatgccacacagacttgcacgcatggcacggggattggcccttaccgactaagttgccgttagtgggcggacatgagggggcgggagtcgtagtgggaatgggagagaacgtgaagggttggaagattggagattatgctgggattaagtggttgaatgggagctgcatggcctgcgaatattgtgaacttggaaatgagagcaattgcccacatgctgacttgtccggttacacacatgacggttcattccaggaatatgctacggctgatgcagtccaagcagcgcatatcccgcaagggacggacttagcagaagtagcgcccattctttgcgctgggatcaccgtatataaagcgttaaagagcgcaaatttacgggccggacattgggcggcgatcagcggggccgcaggggggctgggcagcttggccgtccagtacgctaaagctatgggttatcgggttttgggcattgacggaggaccgggaaaggaggaattattcacgtccttgggaggagaggtattcattgactttaccaaggaaaaagatatcgtctctgctgtagtaaaggctaccaatggcggtgcccacggaatcataaatgtttcagtttctgaagcggcgatcgaagcgtccactagatattgccgtgcaaatgggacagtcgtacttgtaggacttccggctggcgccaaatgcagctccgatgtatttaatcatgtcgtgaagtcaatctctatcgttggttcatatgtaggaaaccgcgccgatactcgtgaggctcttgacttttttgccagaggcctggttaagtcccccataaaagttgttggcttatccagcttacccgaaatatacgagaagatggagaagggccagatcgcggggagatacgttgttgacacttctaaataataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaa

SEQ ID NO: 122 - DNA, 5532 bp, Artificial Sequence
  Synthetic: Pfnrs-LeuDH-kivD-adh2-brnQ construct (with terminator)
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgtctattccagaaacgcagaaagccatcatattttatgaatcgaacggaaaacttgagcacaaggacatccccgtcccgaagccaaaacctaatgagttgcttatcaacgttaagtattcgggcgtatgccacacagacttgcacgcatggcacggggattggcccttaccgactaagttgccgttagtgggcggacatgagggggcgggagtcgtagtgggaatgggagagaacgtgaagggttggaagattggagattatgctgggattaagtggttgaatgggagctgcatggcctgcgaatattgtgaacttggaaatgagagcaattgcccacatgctgacttgtccggttacacacatgacggttcattccaggaatatgctacggctgatgcagtccaagcagcgcatatcccgcaagggacggacttagcagaagtagcgcccattctttgcgctgggatcaccgtatataaagcgttaaagagcgcaaatttacgggccggacattgggcggcgatcagcggggccgcaggggggctgggcagcttggccgtccagtacgctaaagctatgggttatcgggttttgggcattgacggaggaccgggaaaggaggaattattcacgtccttgggaggagaggtattcattgactttaccaaggaaaaagatatcgtctctgctgtagtaaaggctaccaatggcggtgcccacggaatcataaatgtttcagtttctgaagcggcgatcgaagcgtccactagatattgccgtgcaaatgggacagtcgtacttgtaggacttccggctggcgccaaatgcagctccgatgtatttaatcatgtcgtgaagtcaatctctatcgttggttcatatgtaggaaaccgcgccgatactcgtgaggctcttgacttttttgccagaggcctggttaagtcccccataaaagttgttggcttatccagcttacccgaaatatacgagaagatggagaagggccagatcgcggggagatacgttgttgacacttctaaataataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaat

SEQ ID NO: 123 - DNA, 6223 bp, Artificial Sequence
  Synthetic: Tet-LeuDH-kivD-adh2-brnQ construct
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgtctattccagaaacgcagaaagccatcatattttatgaatcgaacggaaaacttgagcacaaggacatccccgtcccgaagccaaaacctaatgagttgcttatcaacgttaagtattcgggcgtatgccacacagacttgcacgcatggcacggggattggcccttaccgactaagttgccgttagtgggcggacatgagggggcgggagtcgtagtgggaatgggagagaacgtgaagggttggaagattggagattatgctgggattaagtggttgaatgggagctgcatggcctgcgaatattgtgaacttggaaatgagagcaattgcccacatgctgacttgtccggttacacacatgacggttcattccaggaatatgctacggctgatgcagtccaagcagcgcatatcccgcaagggacggacttagcagaagtagcgcccattctttgcgctgggatcaccgtatataaagcgttaaagagcgcaaatttacgggccggacattgggcggcgatcagcggggccgcaggggggctgggcagcttggccgtccagtacgctaaagctatgggttatcgggttttgggcattgacggaggaccgggaaaggaggaattattcacgtccttgggaggagaggtattcattgactttaccaaggaaaaagatatcgtctctgctgtagtaaaggctaccaatggcggtgcccacggaatcataaatgtttcagtttctgaagcggcgatcgaagcgtccactagatattgccgtgcaaatgggacagtcgtacttgtaggacttccggctggcgccaaatgcagctccgatgtatttaatcatgtcgtgaagtcaatctctatcgttggttcatatgtaggaaaccgcgccgatactcgtgaggctcttgacttttttgccagaggcctggttaagtcccccataaaagttgttggcttatccagcttacccgaaatatacgagaagatggagaagggccagatcgcggggagatacgttgttgacacttctaaataataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 124 - DNA, 6676 bp, Artificial Sequence
  Synthetic: Tet-LeuDH-kivD-padA-brnQ construct
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgacagagccgcatgtagcagtattaagccaggtccaacagtttctcgatcgtcaacacggtctttatattgatggtcgtcctggccccgcacaaagtgaaaaacggttggcgatctttgatccggccaccgggcaagaaattgcgtctactgctgatgccaacgaagcggatgtagataacgcagtcatgtctgcctggcgggcctttgtctcgcgtcgctgggccgggcgattacccgcagagcgtgaacgtattctgctacgttttgctgatctggtggagcagcacagtgaggagctggcgcaactggaaaccctggagcaaggcaagtcaattgccatttcccgtgcttttgaagtgggctgtacgctgaactggatgcgttataccgccgggttaacgaccaaaatcgcgggtaaaacgctggacttgtcgattcccttaccccagggggcgcgttatcaggcctggacgcgtaaagagccggttggcgtagtggcgggaattgtgccatggaactttccgttgatgattggtatgtggaaggtgatgccagcactggcagcaggctgttcaatcgtgattaagccttcggaaaccacgccactgacgatgttgcgcgtggcggaactggccagcgaggctggtatccctgatggcgtttttaatgtcgtcaccgggtcaggtgctgtatgcggcgcggccctgacgtcacatcctcatgttgcgaaaatcagttttaccggttcaaccgcgacgggaaaaggtattgccagaactgctgctgatcacttaacgcgtgtaacgctggaactgggcggtaaaaacccggcaattgtattaaaagatgctgatccgcaatgggttattgaaggcttgatgaccggaagcttcctgaatcaagggcaagtatgcgccgccagttcgcgaatttatattgaagcgccgttgtttgacacgctggttagtggatttgagcaggcggtaaaatcgttgcaagtgggaccggggatgtcacctgttgcacagattaaccctttggtttctcgtgcgcactgcgacaaagtgtgttcattcctcgacgatgcgcaggcacagcaagcagagctgattcgcgggtcgaatggaccagccggagaggggtattatgttgcgccaacgctggtggtaaatcccgatgctaaattgcgcttaactcgtgaagaggtgtttggtccggtggtaaacctggtgcgagtagcggatggagaagaggcgttacaactggcaaacgacacggaatatggcttaactgccagtgtctggacgcaaaatctctcccaggctctggaatatagcgatcgcttacaggcagggacggtgtgggtaaacagccataccttaattgacgctaacttaccgtttggtgggatgaagcagtcaggaacgggccgtgattttggccccgactggctggacggttggtgtgaaactaagtcggtgtgtgtacggtattaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 125 - DNA, 5622 bp, Artificial Sequence
  Synthetic: LeuDH-kivD-padA-brnQ
atgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgacagagccgcatgtagcagtattaagccaggtccaacagtttctcgatcgtcaacacggtctttatattgatggtcgtcctggccccgcacaaagtgaaaaacggttggcgatctttgatccggccaccgggcaagaaattgcgtctactgctgatgccaacgaagcggatgtagataacgcagtcatgtctgcctggcgggcctttgtctcgcgtcgctgggccgggcgattacccgcagagcgtgaacgtattctgctacgttttgctgatctggtggagcagcacagtgaggagctggcgcaactggaaaccctggagcaaggcaagtcaattgccatttcccgtgcttttgaagtgggctgtacgctgaactggatgcgttataccgccgggttaacgaccaaaatcgcgggtaaaacgctggacttgtcgattcccttaccccagggggcgcgttatcaggcctggacgcgtaaagagccggttggcgtagtggcgggaattgtgccatggaactttccgttgatgattggtatgtggaaggtgatgccagcactggcagcaggctgttcaatcgtgattaagccttcggaaaccacgccactgacgatgttgcgcgtggcggaactggccagcgaggctggtatccctgatggcgtttttaatgtcgtcaccgggtcaggtgctgtatgcggcgcggccctgacgtcacatcctcatgttgcgaaaatcagttttaccggttcaaccgcgacgggaaaaggtattgccagaactgctgctgatcacttaacgcgtgtaacgctggaactgggcggtaaaaacccggcaattgtattaaaagatgctgatccgcaatgggttattgaaggcttgatgaccggaagcttcctgaatcaagggcaagtatgcgccgccagttcgcgaatttatattgaagcgccgttgtttgacacgctggttagtggatttgagcaggcggtaaaatcgttgcaagtgggaccggggatgtcacctgttgcacagattaaccctttggtttctcgtgcgcactgcgacaaagtgtgttcattcctcgacgatgcgcaggcacagcaagcagagctgattcgcgggtcgaatggaccagccggagaggggtattatgttgcgccaacgctggtggtaaatcccgatgctaaattgcgcttaactcgtgaagaggtgtttggtccggtggtaaacctggtgcgagtagcggatggagaagaggcgttacaactggcaaacgacacggaatatggcttaactgccagtgtctggacgcaaaatctctcccaggctctggaatatagcgatcgcttacaggcagggacggtgtgggtaaacagccataccttaattgacgctaacttaccgtttggtgggatgaagcagtcaggaacgggccgtgattttggccccgactggctggacggttggtgtgaaactaagtcggtgtgtgtacggtattaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaa

SEQ ID NO: 126 - DNA, 6135 bp, Artificial Sequence
  Synthetic: Fnrs-LeuDH-kivD-padA-brnQ
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgacagagccgcatgtagcagtattaagccaggtccaacagtttctcgatcgtcaacacggtctttatattgatggtcgtcctggccccgcacaaagtgaaaaacggttggcgatctttgatccggccaccgggcaagaaattgcgtctactgctgatgccaacgaagcggatgtagataacgcagtcatgtctgcctggcgggcctttgtctcgcgtcgctgggccgggcgattacccgcagagcgtgaacgtattctgctacgttttgctgatctggtggagcagcacagtgaggagctggcgcaactggaaaccctggagcaaggcaagtcaattgccatttcccgtgcttttgaagtgggctgtacgctgaactggatgcgttataccgccgggttaacgaccaaaatcgcgggtaaaacgctggacttgtcgattcccttaccccagggggcgcgttatcaggcctggacgcgtaaagagccggttggcgtagtggcgggaattgtgccatggaactttccgttgatgattggtatgtggaaggtgatgccagcactggcagcaggctgttcaatcgtgattaagccttcggaaaccacgccactgacgatgttgcgcgtggcggaactggccagcgaggctggtatccctgatggcgtttttaatgtcgtcaccgggtcaggtgctgtatgcggcgcggccctgacgtcacatcctcatgttgcgaaaatcagttttaccggttcaaccgcgacgggaaaaggtattgccagaactgctgctgatcacttaacgcgtgtaacgctggaactgggcggtaaaaacccggcaattgtattaaaagatgctgatccgcaatgggttattgaaggcttgatgaccggaagcttcctgaatcaagggcaagtatgcgccgccagttcgcgaatttatattgaagcgccgttgtttgacacgctggttagtggatttgagcaggcggtaaaatcgttgcaagtgggaccggggatgtcacctgttgcacagattaaccctttggtttctcgtgcgcactgcgacaaagtgtgttcattcctcgacgatgcgcaggcacagcaagcagagctgattcgcgggtcgaatggaccagccggagaggggtattatgttgcgccaacgctggtggtaaatcccgatgctaaattgcgcttaactcgtgaagaggtgtttggtccggtggtaaacctggtgcgagtagcggatggagaagaggcgttacaactggcaaacgacacggaatatggcttaactgccagtgtctggacgcaaaatctctcccaggctctggaatatagcgatcgcttacaggcagggacggtgtgggtaaacagccataccttaattgacgctaacttaccgtttggtgggatgaagcagtcaggaacgggccgtgattttggccccgactggctggacggttggtgtgaaactaagtcggtgtgtgtacggtattaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 127 - DNA, 6340 bp, Artificial Sequence
  Synthetic: Ptet-LeuDH-kivD-yqhD-brnQ construct
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtactggaatttggcggtattgaaccaaacccggcttatgaaacgctgatgaacgccgtgaaactggttcgcgaacagaaagtgacgttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagcccgtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctgatcgaagatggtccgaaagccctgaaagagccagaaaactacgatgtgcgcgccaacgtcatgtgggcggcgactcaggcgctgaacggtttgatcggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttcagacgatgagcgtattgacgccgcgattgccgcaacccgcaatttctttgagcaattaggcgtgctgacccacctctccgactacggtctggacggcagctccatcccggctttgctgaaaaaactggaagagcacggcatgacccaactgggcgaaaatcatgacattacgctggatgtcagccgccgtatatacgaagccgcccgctaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 128 - DNA, 5286 bp, Artificial Sequence
  Synthetic: LeuDH-kivD-yqhD-brnQ construct
atgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtactggaatttggcggtattgaaccaaacccggcttatgaaacgctgatgaacgccgtgaaactggttcgcgaacagaaagtgacgttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagcccgtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctgatcgaagatggtccgaaagccctgaaagagccagaaaactacgatgtgcgcgccaacgtcatgtgggcggcgactcaggcgctgaacggtttgatcggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttcagacgatgagcgtattgacgccgcgattgccgcaacccgcaatttctttgagcaattaggcgtgctgacccacctctccgactacggtctggacggcagctccatcccggctttgctgaaaaaactggaagagcacggcatgacccaactgggcgaaaatcatgacattacgctggatgtcagccgccgtatatacgaagccgcccgctaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaa

SEQ ID NO: 129 - DNA, 5799 bp, Artificial Sequence
  Synthetic: Pfnrs-LeuDH-kivD-yqhD-brnQ construct
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacatatgactcttgaaatctttgaatatttagaaaagtacgactacgagcaggttgtattttgtcaagacaaggagtctgggctgaaggccatcattgccatccacgacacaaccttaggcccggcgcttggcggaacccgcatgtggacctacgactccgaggaggcggccatcgaggacgcacttcgtcttgctaagggtatgacctataagaacgcggcagccggtctgaatctggggggtgctaagactgtaatcatcggtgatccacgcaaggataagagtgaagcaatgtttcgcgctttagggcgctatattcagggcttgaacggccgctacattaccgcagaagacgtagggacaacagtagacgacatggacatcatccatgaggaaactgatttcgtgaccggtatttcaccttcattcgggtcatccggtaacccttcccccgtaacagcctatggggtttatcgcggaatgaaggccgcagccaaggaggcatttggcactgacaatttagaaggaaaagtaattgctgtccaaggcgtgggcaatgtggcctaccatttgtgtaaacaccttcacgcggaaggtgcaaaattgatcgttacggatattaacaaggaggcagtccagcgcgctgtagaggaatttggagcatcggctgtggaaccaaatgagatctacggtgtagaatgtgacatttacgctccatgcgcacttggtgccacggtgaatgacgagaccatcccccaacttaaggcgaaggtaatcgctggttcagctaacaaccaattaaaagaggaccgtcacggagatatcatccacgaaatgggtatcgtgtacgcccccgattatgttatcaacgcgggcggcgtaatcaacgtagccgatgagctttatggatacaaccgcgaacgtgcgctgaaacgcgtggaaagcatttatgacacgatcgcaaaggtaatcgagatcagtaagcgcgacggcattgcgacatacgtggcagcggaccgtctggccgaagaacgcatcgcgagtttgaagaatagccgtagtacctacttgcgcaacgggcacgatattatcagccgtcgctgataagaaggagatatacatatgtatacagtaggagattacttattggaccggttgcacgaacttggaattgaggaaatttttggagttccgggtgactacaacctgcagttccttgaccaaatcatctcccataaggacatgaaatgggtcggcaatgccaatgagctgaacgcatcatatatggcagacgggtatgctcggaccaaaaaggctgcagcattccttaccacgtttggcgtgggggaattaagtgctgtaaatggactggcaggatcctatgcggagaatttaccggtagtcgaaattgttggctcgcctacgtccaaggtgcagaatgaggggaaattcgtccatcacacacttgcagacggtgattttaagcactttatgaagatgcatgagccggtaacggctgcgcggacgcttcttactgcggaaaacgcaacagtagagattgatcgcgttctgagcgcactgcttaaggaacggaagcccgtctatattaacttaccggtagacgtggccgcagccaaagccgaaaaaccaagcctgcctcttaagaaggagaattccacgtccaacaccagtgaccaagagattttgaacaaaattcaagagtctttgaagaacgcgaagaagcccatcgtaattacaggacatgagattatctcgtttggcctggagaaaacggttacacagtttatttccaaaacgaagttacctataacgacgttaaactttggaaagagctctgtggatgaggcacttcctagtttcttaggaatctataatgggaccctttcagagccaaacttaaaggaattcgttgaaagtgcggattttatcttaatgcttggggttaaattgactgattccagcaccggagcttttacgcaccatttaaacgagaacaaaatgatctctttgaatatcgacgaaggcaaaatttttaatgaaagaattcagaactttgattttgaatcccttattagttcacttttagatttaagtgaaatagagtataagggaaagtatatagacaagaagcaagaggatttcgttccgtctaatgctcttttaagtcaagacagactttggcaggcggttgagaaccttacacaatccaatgaaacgatagtcgccgaacaagggaccagtttcttcggcgcttcttccatattcctgaagtctaagtctcatttcattggacagcccctgtgggggtctataggatatacgtttcccgcagctcttggaagccagatcgccgataaggagagcagacacctgttgttcatcggggacggctcgttgcagctgactgttcaggaactggggttggcgatcagagagaagattaatcccatttgctttatcataaataatgatggttataccgtagaacgtgagattcatggacctaatcagagctataatgacattcctatgtggaactattcaaaattgccagagagttttggtgcaactgaggatcgcgttgttagtaaaatagtccgcacggagaacgagtttgtcagcgtaatgaaggaggcccaagcggaccctaatcggatgtactggatcgaacttattctggctaaagaaggagcacctaaagttttaaagaagatgggaaaactttttgctgaacaaaataaatcataataagaaggagatatacatatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtactggaatttggcggtattgaaccaaacccggcttatgaaacgctgatgaacgccgtgaaactggttcgcgaacagaaagtgacgttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagcccgtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctgatcgaagatggtccgaaagccctgaaagagccagaaaactacgatgtgcgcgccaacgtcatgtgggcggcgactcaggcgctgaacggtttgatcggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttcagacgatgagcgtattgacgccgcgattgccgcaacccgcaatttctttgagcaattaggcgtgctgacccacctctccgactacggtctggacggcagctccatcccggctttgctgaaaaaactggaagagcacggcatgacccaactgggcgaaaatcatgacattacgctggatgtcagccgccgtatatacgaagccgcccgctaataagaaggagatatacatatgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaatacgcatggcatggatgaccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcgtgc

SEQ ID NO: 130 - DNA, 60 bp, Artificial Sequence
  Synthetic: SR36 Primer
tagaactgatgcaaaaagtgctcgacgaaggcacacagatgtgtaggctggagctgcttc

SEQ ID NO: 131 - DNA, 60 bp, Artificial Sequence
  Synthetic: SR38 Primer
gtttcgtaattagatagccaccggcgctttaatgcccggacatatgaatatcctccttag

SEQ ID NO: 132 - DNA, 52 bp, Artificial Sequence
  Synthetic: SR33 Primer
caacacgtttcctgaggaaccatgaaacagtatttagaactgatgcaaaaag

SEQ ID NO: 133 - DNA, 46 bp, Artificial Sequence
  Synthetic: SR34 Primer
cgcacactggcgtcggctctggcaggatgtttcgtaattagatagc

SEQ ID NO: 134 - DNA, 36 bp, Artificial Sequence
  Synthetic: SR43 Primer
atatcgtcgcagcccacagcaacacgtttcctgagg

SEQ ID NO: 135 - DNA, 47 bp, Artificial Sequence
  Synthetic: SR44 Primer
aagaatttaacggagggcaaaaaaaaccgacgcacactggcgtcggc

SEQ ID NO: 136 - DNA, 3383 bp, Artificial Sequence
  Synthetic: Nucleotide sequence of Pfnr1-lacZ construct, low-copy
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaaatgagcaaagtcagactcgcaattatggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 137 - DNA, 3258 bp, Artificial Sequence
  Synthetic: Nucleotide sequences of Pfnr2-lacZ construct, low-copy
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacctatgacgacctacgatcgggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 138 - DNA, 3386 bp, Artificial Sequence
  Synthetic: Nucleotide sequences of Pfnr3-lacZ construct, low-copy
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 139 - DNA, 3261 bp, Artificial Sequence
  Synthetic: Nucleotide sequences of Pfnr4-lacZ construct, low-copy
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 140 - DNA, 3279 bp, Artificial Sequence
  Synthetic: Nucleotide sequences of Pfnrs-lacZ construct, low-copy
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacatatgctatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 141 - DNA, 967 bp, Artificial Sequence
  Synthetic: Wild-type clbA
caaatatcacataatcttaacatatcaataaacacagtaaagtttcatgtgaaaaacatcaaacataaaatacaagctcggaatacgaatcacgctatacacattgctaacaggaatgagattatctaaatgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattgaatcgccacggataatctagacacttctgagccgtcgataatattgattttcatattccgtcggtggtgtaagtatcccgcataatcgtgccattcacatttag

SEQ ID NO: 142 - DNA, 424 bp, Artificial Sequence
  Synthetic: clbA knockout
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 143 - DNA, 200 bp, Artificial Sequence
  Synthetic: FNR-responsive regulatory region Sequence: fnrS+crp
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 144 - DNA, 18 bp, Artificial Sequence
  Synthetic: consensus sequence
ttgttgayryrtcaacwa

(19) United States
(12) Patent Application Publication
(10) Pub. No.: US 2017/0232043 A1
(43) Pub. Date: Aug. 17, 2017

(54) BACTERIA ENGINEERED TO TREAT DISORDERS INVOLVING THE CATABOLISM OF A BRANCHED CHAIN AMINO ACID

(71) Applicant: Synlogic, Inc., Cambridge, MA (US)

(72) Inventors: Dean Falb, Sherborn, MA (US); Paul F. Miller, Salem, CT (US); Yves Millet, Newton, MA (US); Vincent M. Isabella, Cambridge, MA (US); Jonathan W. Kotula, Somerville, MA (US); Alex Tucker, Somerville, MA (US)

(21) Appl. No.: 15/379,445

(22) Filed: Dec. 14, 2016

Related U.S. Application Data
(63) Continuation-in-part of application No. PCT/US2016/037098, filed on Jun. 10, 2016.
Application No. PCT/US2016/037098 is a continuation of application No. PCT/US2016/032565, filed on May 13, 2016.
(60) Provisional application No. 62/336,338, filed on May 13, 2016.
(60) Provisional application No. 62/173,761, filed on Jun. 10, 2015.

(51) Int. Cl. A61K 35/74; A61K 38/44; C12N 9/88; C12N 15/70; C12N 9/10; C12N 9/04; C07K 14/245; A61K 9/00; C12N 9/06

(52) U.S. Cl. CPC A61K 35/74; A61K 9/0053; A61K 38/44; A61K 38/443; A61K 9/0019; C12Y 104/03002; C12Y 104/01009; C12N 9/88; C12N 9/0016; C12N 9/1096; C12N 9/0022; C12N 9/0006; C12Y 101/01; C12Y 401/01; C07K 14/245; C12Y 101/01086; C12Y 401/01001; C12Y 206/01042; C12Y 101/01001; C12N 15/70

(57) ABSTRACT

The present disclosure provides recombinant bacterial cells that have been engineered with genetic circuitry which allow the recombinant bacterial cells to sense a patient's internal environment and respond by turning an engineered metabolic pathway on or off. When turned on, the recombinant bacterial cells complete all of the steps in a metabolic pathway to achieve a therapeutic effect in a host subject. These recombinant bacterial cells are designed to drive therapeutic effects throughout the body of a host from a point of origin of the microbiome. Specifically, the present disclosure provides recombinant bacterial cells comprising a heterologous gene encoding a branched chain amino acid catabolism enzyme. The disclosure further provides pharmaceutical compositions comprising the recombinant bacteria, and methods for treating disorders involving the catabolism of branched chain amino acids using the pharmaceutical compositions disclosed herein.

### RELATED APPLICATIONS

This application is a continuation-in-part of PCT Application No. PCT/US2016/037098, filed Jun. 10, 2016, which claims priority to U.S. Provisional Patent Application No. 62/173,761, filed on Jun. 10, 2015, U.S. Provisional Patent Application No. 62/336,338, filed May 13, 2016, PCT Application No. PCT/US2016/032565, filed May 13, 2016, the entire contents of each of which are expressly incorporated by reference.

### SEQUENCE LISTING

The instant application contains a Sequence Listing which has been submitted electronically in ASCII format and is hereby incorporated by reference in its entirety. Said ASCII copy, created on Feb. 24, 2017, is named 126046-00204_SL.txt and is 420,301 bytes in size.

### BACKGROUND

The branched chain amino acids (BCAAs), e.g., leucine, isoleucine, and valine, play an important role in the metabolism of living organisms. Transamination of branched chain amino acids gives rise to their corresponding branched chain α-keto acids (BCKAs) (α-keto-β-methylvalerate, α-ketoisocaproate, and α-ketoisovalerate), which undergo further oxidative decarboxylation to produce acyl-CoA derivatives that enter the TCA cycle. Branched chain amino acids provide a nonspecific carbon source of oxidation for production of energy and also act as a precursor for muscle protein synthesis (Monirujjaman and Ferdouse, Advances in Molec. Biol., 2014, Article ID 36976, 6 pages, 2014).

Enzyme deficiencies or mutations which lead to the toxic accumulation of branched chain amino acids and their corresponding alpha-keto acids in the blood, cerebrospinal fluid, and tissues result in the development of metabolic disorders associated with the abnormal catabolism of branched chain amino acids in subjects, such as maple syrup urine disease (MSUD), isovaleric acidemia, propionic acidemia, methylmalonic acidemia, and diabetes ketoacidosis. Clinical manifestations of the disease vary depending on the degree of enzyme deficiency and include neurological dysfunction, seizures and death (Homanics et al. 2009).

Branched chain amino acids, such as leucine, or their corresponding alpha-keto acids, have also been linked to mTor activation (see, for example, Harlan et al., Cell Metabolism, 17:599-606, 2013) which is, in turn, associated with diseases such as cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia (see Laplante and Sabatini, Cell, 149(2):74-293, 2012).

Currently available treatments for disorders involving the catabolism of branched chain amino acids are inadequate for the long term management of the disorders and have severe limitations (Svkvorak, J. Inherit. Metab. Dis., 32(2):229-246, 2009). A low protein/BCAA-restricted diet, with micronutrient and vitamin supplementation, as necessary, is the widely accepted long-term disease management strategy for many such disorders (Homanics et al., BMC Med. Genet., 7:33, 2006). However, BCAA-intake restrictions can be particularly problematic since branched chain amino acids can only be acquired through diet and are necessary for metabolic activities including protein synthesis and branched-chain fatty acid synthesis (Skvorak, 2009). Thus, even with proper monitoring and patient compliance, branched chain amino acid dietary restrictions result in a high incidence of mental retardation and mortality (Skvorak, 2009; Homanics et al., 2009). A few cases of MSUD have been treated by liver transplantation (Popescu and Dima, Liver Transpl., 1:22-28, 2012). However, the limited availability of donor organs, the costs associated with the transplantation itself, and the undesirable effects associated with continued immunosuppressant therapy limit the practicality of liver transplantation for treatment of disorders involving the catabolism of a branched chain amino acid (Homanics et al., 2012; Popescu and Dima, 2012). Therefore, there is significant unmet need for effective, reliable, and/or long-term treatment for disorders involving the catabolism of branched chain amino acids.

### SUMMARY

The present disclosure relates to compositions and therapeutic methods for reducing one or more excess branched chain amino acids, and/or an accumulated metabolite(s) thereof, for example, by converting the one or more excess branched chain amino acid(s) or accumulated metabolite(s) into alternate by product(s). In certain aspects, the disclosure relates to genetically engineered microorganisms, e.g., bacteria, yeast or viruses, that are capable of reducing one or more excess branched chain amino acids, and/or an accumulated metabolite(s) thereof, particularly in low-oxygen conditions, such as in the mammalian gut. In certain aspects, the compositions and methods disclosed herein may be used for modulating excess branched chain amino acids and/or an accumulated metabolite(s) thereof. In certain aspects, the compositions and methods disclosed herein may be used to treat disorders associated with excess branched chain amino acids and/or an accumulated metabolite(s) thereof, e.g., MSUD, isovaleric acidemia (IVA), propionic acidemia, methylmalonic acidemia, and diabetes ketoacidosis, as well as other diseases, for example, 3-MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria. In certain aspects, the compositions and methods disclosed herein may be used to treat disorders associated with excess branched chain amino acids, such as leucine, and/or an accumulated metabolite(s) thereof, e.g., corresponding alpha-keto acids of BCAA, which are associated with diseases such as cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia.

In certain aspects, the invention provides genetically engineered bacteria that are capable of reducing one or more branched chain amino acids (BCAA) or metabolite(s) thereof. In some aspects, the engineered bacteria can convert the BCAA, or metabolite thereof, into one or more alternate byproduct(s). In some aspects, the branched chain amino acid(s) or metabolite(s) thereof are present in excess amount(s) compared with a normal or reference range amount. For example, in certain aspects, the invention provides genetically engineered bacteria that are capable of reducing one or more leucine, isoleucine, and/or valine or a metabolite(s) thereof, including, for example, α-ketoisocaproate, α-keto-β-methylvalerate, and/or α-ketovalerate. In certain embodiments, the genetically engineered bacteria reduce excess BCAA and convert BCAA, or one or more metabolites thereof, into alternate byproducts selectively in low-oxygen environments, e.g., in the gut. In certain embodiments, the genetically engineered bacteria are non-pathogenic and may be introduced into the gut in order to reduce excess levels of BCAA. Another aspect of the invention provides methods for selecting or targeting genetically engineered bacteria based on increased levels of BCAA or metabolite consumption, and/or increase of uptake of branched chain amino acid into the bacterial cell. The invention also provides pharmaceutical compositions comprising the genetically engineered bacteria, methods for modulating the levels of BCAA(s), e.g., reducing excess levels of BCAA(s), and methods for treating diseases or disorders associated with one or more excess BCAA(s), e.g., MSUD, isovaleric acidemia (IVA), propionic acidemia, methylmalonic acidemia, diabetes ketoacidosis, 3-MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria.

The present disclosure provides recombinant microorganisms that have been engineered with genetic circuitry which allow the recombinant microorganism to import and/or metabolize one or more branched chain amino acid and/or one or more metabolite(s) thereof. In some embodiments, the engineered microorganism is capable of sensing a patient's internal environment, e.g., the gut, and responding by turning an engineered metabolic pathway on or off. When turned on, the engineered microorganism, e.g., bacterial, yeast or virus cell, expresses one or more enzymes in a metabolic pathway to achieve a therapeutic effect in a host subject.

In certain aspects, the present disclosure provides engineered bacterial cells, pharmaceutical compositions thereof, and methods of modulating BCAA(s) and/or metabolite(s) thereof and treating diseases associated with the catabolism of branched chain amino acids. Specifically, the engineered bacteria disclosed herein have been modified to comprise gene sequence(s) encoding one or more enzymes involved in branched chain amino acid catabolism, as well as other circuitry, e.g., to regulate gene expression, including, for example, sequences for one or more inducible promoter(s), sequences for importing one or more BCAA(s) and/or metabolite(s) thereof into the bacterial cell (e.g., transporter sequence(s)), sequences for the secretion or non-secretion of BCAA(s), metabolites or by-products (e.g., exporter(s) or exporter knockouts), and circuitry to guarantee the safety and non-colonization of the subject that is administered the recombinant bacteria, such as auxotrophies, kill switches, etc. These engineered bacteria are safe and well tolerated and augment the innate activities of the subject's microbiome to achieve a therapeutic effect.

In some embodiments, the present disclosure provides a bacterium comprising gene sequence(s) encoding one or more branched chain amino acid catabolism enzyme(s). In some embodiments, the present disclosure provides a bacterium comprising gene sequence(s) encoding one or more branched chain amino acid catabolism enzyme(s) operably linked to a directly or indirectly inducible promoter that is not associated with the branched chain amino acid catabolism enzyme gene in nature. In some embodiments, the present disclosure provides a bacterium comprising gene sequence(s) encoding one or more branched chain amino acid catabolism enzyme(s) that are capable of converting a branched chain amino acid α-ketoacid, e.g., α-ketoisocaproate, α-keto-β-methylvalerate, and/or α-ketoisovalerate to its corresponding branched chain amino acid aldehyde, e.g., isovaleraldehyde, 2-methylbutyraldehyde, and/or isobutyraldehyde, respectively. In some embodiments, the bacterium comprises gene sequence(s) encoding one or more α-ketoacid decarboxylase(s). In some embodiments, the α-ketoacid decarboxylase is KivD, e.g., the bacterium comprises gene sequence(s) encoding one or more kivD genes. In some embodiments, the kivD gene is derived from a Lactococcus lactis, e.g., lactococcus lactis IFPL730.

In some embodiments, the bacterium comprises gene sequence(s) encoding one or more branched chain amino acid catabolism enzyme(s) that are capable of converting leucine, isoleucine and/or valine to α-ketoisocaproate, α-keto-β-methylvalerate, and/or α-ketoisovalerate, respectively. In some embodiments, the bacterium comprises gene sequence(s) encoding one or more branched chain amino acid deamination enzymes. In some embodiments, the branched chain amino acid deamination enzyme is selected from a branched chain amino acid dehydrogenase, branched chain amino acid aminotransferase, and amino acid oxidase. Thus, in some embodiments, the bacterium comprises gene sequence(s) encoding a gene selected from a branched chain amino acid dehydrogenase, branched chain amino acid aminotransferase, and amino acid oxidase. In some embodiments, the branched chain amino acid dehydrogenase is leucine dehydrogenase. In some embodiments, the leucine dehydrogenase is a bacillus cereus leucine dehydrogenase. In some embodiments, the branched chain amino acid aminotransferase is ilvE. In some embodiments, the amino acid oxidase is L-AAD. In some embodiments, the L-AAD gene is derived from proteus vulgaris or proteus mirabilis.

In some embodiments, the present disclosure provides a bacterium comprising gene sequence(s) encoding one or more branched chain amino acid catabolism enzyme(s) that are capable of converting a branched chain amino acid α-ketoacid, e.g., α-ketoisocaproate, α-keto-β-methylvalerate, and/or α-ketoisovalerate to its corresponding branched chain amino acid aldehyde, e.g., isovaleraldehyde, 2-methylbutyraldehyde, and/or isobutyraldehyde, respectively and gene sequence(s) encoding one or more branched chain amino acid catabolism enzyme(s) that are capable of converting leucine, isoleucine and/or valine to α-ketoisocaproate, α-keto-β-methylvalerate, and/or α-ketoisovalerate, respectively. In some embodiments, the branched chain amino acid catabolism enzyme(s) capable of converting a branched chain amino acid α-ketoacid, to its corresponding branched chain amino acid aldehyde is a α-ketoacid decarboxylase. In some embodiments, the α-ketoacid decarboxylase is KivD, e.g., the bacterium comprises gene sequence(s) encoding one or more kivD genes. In some embodiments, the kivD gene is derived from a Lactococcus lactis, e.g., lactococcus lactis IFPL730. In some embodiments, the branched chain amino acid catabolism enzyme(s) capable of converting leucine, isoleucine and/or valine to their corresponding branched chain α-ketoacids is a branched chain amino acid deamination enzyme. In some embodiments, the branched chain amino acid deamination enzyme is a branched chain amino acid dehydrogenase, branched chain amino acid aminotransferase, and/or amino acid oxidase. Thus, in some embodiments, the bacterium comprises gene sequence(s) encoding a gene selected from a branched chain amino acid dehydrogenase, branched chain amino acid aminotransferase, and amino acid oxidase. In some embodiments, the branched chain amino acid dehydrogenase is leucine dehydrogenase. In some embodiments, the leucine dehydrogenase is a bacillus cereus leucine dehydrogenase. In some embodiments, the branched chain amino acid aminotransferase is ilvE. In some embodiments, the amino acid oxidase is L-AAD. In some embodiments, the L-AAD gene is derived from proteus vulgaris or proteus mirabilis.

In some embodiments, the bacterium comprises gene sequence(s) encoding one or more branched chain amino acid catabolism enzyme(s) that are capable of converting a branched chain amino acid aldehyde, e.g., isovaleraldehyde, isobutyraldehyde and/or 2-methylbutyraldehyde to its corresponding alcohol, e.g., isopentanol, isobutanol, and/or 2-methybutanol, respectively. In some embodiments, the bacterium comprises gene sequence(s) encoding one or more branched chain amino acid alcohol dehydrogenases. In some embodiments, the branched chain amino acid alcohol dehydrogenase gene is selected from adh1, adh2, adh2, adh4, adh5, adh6, adh7, sfa1, and yqhD. In some embodiments, the branched chain amino acid alcohol dehydrogenase gene is adh2. In some embodiments, the adh2 is derived from S. cerevisiae adh2. In some embodiments, the branched chain amino acid alcohol dehydrogenase gene is yqhD. In some embodiments, the yqhD gene is derived from E. Coli. In any of these embodiments wherein the bacteria comprises gene sequence(s) encoding a branched chain amino acid alcohol dehydrogenase, the bacterium may further comprise gene sequence(s) encoding one or more branched chain amino acid catabolism enzyme(s) that are capable of converting a branched chain amino acid α-ketoacid to its corresponding branched chain amino acid aldehyde and/or gene sequence(s) encoding one or more branched chain amino acid catabolism enzyme(s) that are capable of converting leucine, isoleucine and/or valine to α-ketoisocaproate, α-keto-β-methylvalerate, and/or α-ketoisovalerate, respectively. In some embodiments, the α-ketoacid decarboxylase is KivD, e.g., the bacterium comprises gene sequence(s) encoding one or more kivD genes. In some embodiments, the branched chain amino acid deamination enzyme is a branched chain amino acid dehydrogenase, branched chain amino acid aminotransferase, and/or amino acid oxidase. Thus, in some embodiments, the bacterium comprises gene sequence(s) encoding a gene selected from a branched chain amino acid dehydrogenase, branched chain amino acid aminotransferase, and amino acid oxidase. In some embodiments, the branched chain amino acid dehydrogenase is leucine dehydrogenase, e.g., derived from bacillus cereus. In some embodiments, the branched chain amino acid aminotransferase is ilvE. In some embodiments, the amino acid oxidase is L-AAD, e.g., derived from proteus vulgaris or proteus mirabilis.

In some embodiments, the bacterium comprises gene sequence(s) encoding one or more branched chain amino acid catabolism enzyme(s) that are capable of converting isovaleraldehyde, isobutyraldehyde and/or 2-methylbutyraldehyde to isovalerate, isobutyrate, and/or 2-methybutyrate, respectively. In some embodiments, the branched chain amino acid catabolism enzyme that is capable of converting isovaleraldehyde, isobutyraldehyde and/or 2-methylbutyraldehyde to it corresponding branched chain amino acid carboxylic acid is an aldehyde dehydrogenase. Thus, in some embodiments, the bacterium comprises gene sequence(s) encoding one or more branched chain amino acid aldehyde dehydrogenases.

In some embodiments, the branched chain amino acid aldehyde dehydrogenase gene is padA. In some embodiments, the padA is an E. Coli padA. In any of these embodiments wherein the bacteria comprises gene sequence(s) encoding a branched chain amino acid aldehyde dehydrogenase, the bacterium may further comprise gene sequence(s) encoding one or more branched chain amino acid catabolism enzyme(s) that are capable of converting a branched chain amino acid α-ketoacid to its corresponding branched chain amino acid aldehyde and/or gene sequence(s) encoding one or more branched chain amino acid catabolism enzyme(s) that are capable of converting leucine, isoleucine and/or valine to α-ketoisocaproate, α-keto-β-methylvalerate, and/or α-ketoisovalerate, respectively, and/or gene sequence(s) encoding one or more branched chain amino acid catabolism enzyme(s) that are capable of converting a branched chain amino acid aldehyde to its corresponding alcohol. In some embodiments, the α-ketoacid decarboxylase is KivD, e.g., the bacterium comprises gene sequence(s) encoding one or more kivD genes. In some embodiments, the branched chain amino acid deamination enzyme is a branched chain amino acid dehydrogenase, branched chain amino acid aminotransferase, and/or amino acid oxidase. Thus, in some embodiments, the bacterium comprises gene sequence(s) encoding a gene selected from a branched chain amino acid dehydrogenase, branched chain amino acid aminotransferase, and amino acid oxidase. In some embodiments, the branched chain amino acid dehydrogenase is leucine dehydrogenase, e.g., derived from bacillus cereus. In some embodiments, the branched chain amino acid aminotransferase is ilvE. In some embodiments, the amino acid oxidase is L-AAD, e.g., derived from proteus vulgaris or proteus mirabilis. In some embodiments, the bacterium comprises gene sequence(s) encoding one or more branched chain amino acid alcohol dehydrogenases, e.g., selected from adh1, adh2, adh3, adh4, adh5, adh6, adh7, sfa1, and yqhD. In some embodiments, the branched chain amino acid alcohol dehydrogenase gene is adh2. In some embodiments, the adh2 is derived from S. cerevisiae adh2. In some embodiments, the branched chain amino acid alcohol dehydrogenase gene is yqhD. In some embodiments, the yqhD gene is derived from E. Coli.

In some embodiments, the bacterium comprises gene sequence(s) encoding one or more transporter(s) of a branched chain amino acid. In some embodiments, the bacterium comprises gene sequence(s) encoding one or more transporter(s) of a branched chain amino acid operably linked to a promoter that is not associated with the transporter gene in nature. In some embodiments, the promoter is a directly or indirectly inducible promoter. In some embodiments, the transporter of branched chain amino acid is selected from livKHMGF and brnQ. In some embodiments, the livKHMGF is an E. Coli livKHMGF gene. In some embodiments, the gene sequence encoding one or more transporters is present in a chromosome in the bacteria. In some embodiments, the gene sequence encoding one or more transporters is present in one or more plasmids.

In some embodiments, the bacterium comprises gene sequence(s) encoding one or more transporter(s) of a branched chain amino acid and gene sequence encoding one or more branched chain amino acid catabolism enzymes. In some embodiments, in which the bacterium comprises gene sequence(s) encoding one or more transporter(s) of a branched chain amino acid and gene sequence encoding one or more branched chain amino acid catabolism enzymes, the branched chain amino acid catabolism enzyme(s) is capable of converting α-ketoisocaproate, α-keto-β-methylvalerate, and/or α-ketoisovalerate to isovaleraldehyde, 2-methylbutyraldehyde, and/or isobutyraldehyde, respectively, e.g., the bacterium comprises gene sequence(s) encoding one or more α-ketoacid decarboxylase(s). In some embodiments, the α-ketoacid decarboxylase is kivD. In some embodiments, in which the bacterium comprises gene sequence(s) encoding one or more transporter(s) of a branched chain amino acid and gene sequence encoding one or more branched chain amino acid catabolism enzymes, the branched chain amino acid catabolism enzyme(s) is capable of converting leucine, isoleucine and/or valine to α-ketoisocaproate, α-keto-β-methylvalerate, and/or α-ketoisovalerate, respectively, e.g., the bacterium comprises gene sequence(s) encoding one or more branched chain amino acid deamination enzymes, for example, selected from a branched chain amino acid dehydrogenase, branched chain amino acid aminotransferase, and amino acid oxidase. In some embodiments, the branched chain amino acid dehydrogenase is leucine dehydrogenase. In some embodiments, the branched chain amino acid aminotransferase is ilvE. In some embodiments, the amino acid oxidase is L-AAD. In some embodiments, in which the bacterium comprises gene sequence(s) encoding one or more transporter(s) of a branched chain amino acid and gene sequence encoding one or more branched chain amino acid catabolism enzymes, the branched chain amino acid catabolism enzyme(s) is capable of converting isovaleraldehyde, isobutyraldehyde and/or 2-methylbutyraldehyde to isopentanol, isobutanol, and/or 2-methybutanol, respectively, e.g., is a branched chain amino acid alcohol dehydrogenases, for example, selected from adh1, adh2, adh3, adh4, adh5, adh6, adh7, sfa1, and yqhD and/or is capable of converting isovaleraldehyde, isobutyraldehyde and/or 2-methylbutyraldehyde to isovalerate, isobutyrate, and/or 2-methybutyrate, respectively, e.g., is a branched chain amino acid aldehyde dehydrogenase, for example, padA.

Thus, in some embodiments, the bacterium comprising gene sequence(s) encoding one or more transporters of branched chain amino acids, e.g., livKHMGF and/or brnQ and gene sequence(s) encoding one or more α-ketoacid decarboxylase(s), e.g., kivD. In some embodiments, the bacterium comprising gene sequence(s) encoding one or more transporters of branched chain amino acids, e.g., livKHMGF and/or brnQ, and gene sequence(s) encoding kivD. In some embodiments, the bacterium comprising gene sequence(s) encoding one or more transporters of branched chain amino acids, e.g., livKHMGF and/or brnQ, and gene sequence(s) encoding one or more branched chain amino acid deamination enzymes, for example, selected from a branched chain amino acid dehydrogenase, e.g., LeuDH, branched chain amino acid aminotransferase, e.g., ilvE, and amino acid oxidase, e.g., L-AAD. In some embodiments, the bacterium comprising gene sequence(s) encoding one or more transporters of branched chain amino acids, e.g., livKHMGF and/or brnQ, and gene sequence(s) encoding one or more α-ketoacid decarboxylase(s), e.g., kivD and gene sequence(s) encoding a branched chain amino acid dehydrogenase, e.g., LeuDH, a branched chain amino acid aminotransferase, e.g., ilvE, and/or an amino acid oxidase, e.g., L-AAD. Thus, in some embodiments, the bacterium comprise gene sequence(s) encoding one or more transporters of branched chain amino acids, e.g., livKHMGF and/or brnQ, gene sequence(s) encoding kivD, and gene sequence(s) encoding LeuDH. In some embodiments, the bacterium comprise gene sequence(s) encoding one or more transporters of branched chain amino acids, e.g., livKHMGF and/or brnQ, gene sequence(s) encoding kivD, and gene sequence(s) encoding LeuDH and/or ilvE, and/or L-AAD. In some embodiments, the bacterium comprising gene sequence(s) encoding one or more transporters of branched chain amino acids, e.g., livKHMGF and/or brnQ, and gene sequence(s) encoding one or more branched chain amino acid alcohol dehydrogenases, for example, selected from adh1, adh2, adh3, adh4, adh5, adh6, adh7, sfa1, and yqhD. In some embodiments, the bacterium comprising gene sequence(s) encoding one or more transporters of branched chain amino acids, e.g., livKHMGF and/or brnQ, gene sequence(s) encoding one or more branched chain amino acid deamination enzymes, for example, selected from a branched chain amino acid dehydrogenase, e.g., LeuDH, branched chain amino acid aminotransferase, e.g., ilvE, and amino acid oxidase, e.g., L-AAD, and gene sequence(s) encoding one or more branched chain amino acid aldehyde dehydrogenase, for example, padA and/or gene sequence(s) encoding one or more branched chain amino acid alcohol dehydrogenases, for example, selected from adh1, adh2, adh3, adh4, adh5, adh6, adh7, sfa1, and yqhD. In some embodiments, the bacterium comprising gene sequence(s) encoding one or more transporters of branched chain amino acids, e.g., livKHMGF and/or brnQ, gene sequence(s) encoding one or more α-ketoacid decarboxylase(s), e.g., kivD, and gene sequence(s) encoding one or more branched chain amino acid aldehyde dehydrogenase, for example, padA and/or gene sequence(s) encoding one or more branched chain amino acid alcohol dehydrogenases, for example, selected from adh1, adh2, adh3, adh4, adh5, adh6, adh7, sfa1, and yqhD. In some embodiments, the bacterium comprising gene sequence(s) encoding one or more transporters of branched chain amino acids, e.g., livKHMGF and/or brnQ, gene sequence(s) encoding one or more α-ketoacid decarboxylase(s), e.g., kivD, gene sequence(s) encoding one or more branched chain amino acid deamination enzymes, for example, selected from a branched chain amino acid dehydrogenase, e.g., LeuDH, branched chain amino acid aminotransferase, e.g., ilvE, and amino acid oxidase, e.g., L-AAD, and gene sequence(s) encoding one or more branched chain amino acid aldehyde dehydrogenase, for example, padA and/or gene sequence(s) encoding one or more branched chain amino acid alcohol dehydrogenases, for example, selected from adh1, adh2, adh3, adh4, adh5, adh6, adh7, sfa1, and yqhD. Thus, in some embodiments the bacterium comprises gene sequence(s) encoding one or more transporters of branched chain amino acids, e.g., livKHMGF and/or brnQ, gene sequence(s) encoding kivD, and gene sequence(s) encoding LeuDH and/or ilvE, and/or L-AAD. In some embodiments, the bacterium comprises gene sequence(s) encoding one or more transporters of branched chain amino acids, e.g., livKHMGF and/or brnQ, gene sequence(s) encoding kivD, and gene sequence(s) encoding LeuDH. In some embodiments, the bacterium comprises gene sequence(s) encoding one or more transporters of branched chain amino acids, e.g., livKHMGF and/or brnQ, gene sequence(s) encoding kivD, gene sequence(s) encoding LeuDH and/or ilvE, and/or L-AAD, and gene sequence encoding padA. In some embodiments, the bacterium comprises gene sequence(s) encoding one or more transporters of branched chain amino acids, e.g., livKHMGF and/or brnQ, gene sequence(s) encoding kivD, gene sequence(s) encoding LeuDH, and gene sequence encoding padA. In some embodiments, the bacterium comprises gene sequence(s) encoding one or more transporters of branched chain amino acids, e.g., livKHMGF and/or brnQ, gene sequence(s) encoding kivD, gene sequence(s) encoding LeuDH and/or ilvE, and/or L-AAD, and gene sequence encoding adh2 or yqhD. In some embodiments, the bacterium comprises gene sequence(s) encoding one or more transporters of branched chain amino acids, e.g., livKHMGF and/or brnQ, gene sequence(s) encoding kivD, gene sequence(s) encoding LeuDH, and gene sequence encoding adh2 or yqhD. In some embodiments, the bacterium comprising gene sequence(s) encoding one or more transporters of branched chain amino acids, e.g., livKHMGF and/or brnQ, gene sequence(s) encoding kivD, gene sequence(s) encoding LeuDH and/or ilvE, and/or L-AAD, gene sequence encoding adh2 or yqhD, and gene sequence encoding padA. In some embodiments, the bacterium comprising gene sequence(s) encoding one or more transporters of branched chain amino acids, e.g., livKHMGF and/or brnQ, gene sequence(s) encoding kivD, gene sequence(s) encoding LeuDH, gene sequence encoding adh2 or yqhD, and gene sequence encoding padA.

In any of these embodiments, the bacterium further comprises a genetic modification that reduces export of a branched chain amino acid from the bacterium. In some embodiments, the genetic modification that reduces export of a branched chain amino acid from the bacterium is gene modification in the leuE gene, for example, the leuE gene is deleted from the bacterium. In any of these embodiments, the bacterium further comprises a genetic modification that reduces endogenous biosynthesis of a branched chain amino acid in the bacterium. In some embodiments, the genetic modification that reduces endogenous biosynthesis of a branched chain amino acid in the bacterium is a gene modification in the ilvC gene, e.g., the ilvC gene is deleted from the bacterium. In any of these embodiments, the bacterium further comprises gene sequence(s) encoding one or more branched chain amino acid binding protein(s), e.g., further comprises gene sequence(s) encoding ilvJ.

In any of these embodiments, wherein the promoter operably linked to the gene sequence(s) encoding a branched chain amino acid catabolism enzyme and the promoter operably linked to the gene sequence(s) encoding a transporter of a branched chain amino acid are separate copies of the same promoter. In some embodiments, the promoter operably linked to the gene sequence(s) encoding a branched chain amino acid catabolism enzyme and the promoter operably linked to the gene sequence(s) encoding a transporter of a branched chain amino acid are the same copy of the same promoter. Ins some embodiments, the promoter operably linked to the gene sequence(s) encoding a branched chain amino acid catabolism enzyme and the promoter operably linked to the gene sequence(s) encoding a transporter of a branched chain amino acid are different promoters. In some embodiment, the promoter operably linked to the gene sequence(s) encoding a branched chain amino acid catabolism enzyme is directly or indirectly induced by exogenous environmental conditions found in the mammalian gut. In some embodiments, the promoter operably linked to the gene sequence(s) encoding a branched chain amino acid catabolism enzyme is directly or indirectly induced under low-oxygen or anaerobic conditions. In some embodiments, the promoter operably linked to the gene sequence(s) encoding a branched chain amino acid catabolism enzyme is selected from the group consisting of an FNR-responsive promoter, an ANR-responsive promoter, and a DNR-responsive promoter. In some embodiments, the promoter operably linked to the gene sequence(s) encoding a branched chain amino acid catabolism enzyme is an FNRS promoter. In some embodiments, he promoter operably linked to the gene sequence(s) encoding a transporter of a branched chain amino acid is directly or indirectly induced by exogenous environmental conditions found in the mammalian gut. In some embodiments, the promoter operably linked to the gene sequence(s) encoding a transporter of a branched chain amino acid is directly or indirectly induced under low-oxygen or anaerobic conditions. In some embodiments, the promoter operably linked to the gene sequence(s) encoding a transporter of a branched chain amino acid is selected from the group consisting of an FNR-responsive promoter, an ANR-responsive promoter, and a DNR-responsive promoter. In some embodiments, the promoter operably linked to the gene sequence(s) encoding a transporter of a branched chain amino acid catabolism enzyme is an FNRS promoter.

In some embodiments, the gene sequence(s) encoding a branched chain amino acid catabolism enzyme is located on a chromosome in the bacterium. In some embodiments, the gene sequence(s) encoding a branched chain amino acid catabolism enzyme is located on a plasmid in the bacterium. In some embodiments, at least one gene sequence(s) encoding a branched chain amino acid catabolism enzyme is located on a plasmid in the bacterium and at least one gene sequence(s) encoding a branched chain amino acid catabolism enzyme is located on a chromosome in the bacterium. In some embodiments, the gene sequence(s) encoding a transporter of a branched chain amino acid is located on a chromosome in the bacterium. In some embodiments, the gene sequence(s) encoding a transporter of a branched chain amino acid is located on a plasmid in the bacterium.

In some embodiments, the gene sequence(s) encoding a transporter of a branched chain amino acid is located on a plasmid in the bacterium and at least one gene sequence(s) encoding a transporter of a branched chain amino acid is located on a chromosome in the bacterium.

In any of these embodiments, the bacterium is an auxotroph in diaminopimelic acid or an enzyme in the thymidine biosynthetic pathway. In any of these embodiments, the bacterium is further engineered to harbor a gene encoding a substance toxic to the bacterium, wherein the gene is under the control of a promoter that is directly or indirectly induced by an environmental factor not naturally present in a mammalian gut.

The present disclosure provides a method of reducing the level of a branched amino acid or treating a disease associated with excess branched chain amino acid comprising the step of administering to a subject in need thereof, a composition comprising any of the bacterium described herein. In some embodiments, the disclosure provides a method of reducing the level of a branched amino acid metabolite or treating a disease associated with excess branched chain amino acid metabolite comprising the step of administering to a subject in need thereof, a composition comprising any of the bacterium described herein. In some embodiments, the branch chain amino acid metabolite is selected from α-ketoisocaproate, α-keto-β-methylyalerate, and α-ketoisovalerate. In some embodiments, the disease is selected from the group consisting of: MSUD, isovaleric acidemia (IVA), propionic acidemia, methylmalonic acidemia, and diabetes ketoacidosis, as well as other diseases, for example, 3-MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria. In some embodiments, the disclosure provides a method for treating a metabolic disorder involving the abnormal catabolism of a branched amino acid in a subject, the method comprising administering a composition comprising any of the bacterium described herein and thereby treating the metabolic disorder involving the abnormal catabolism of a branched chain amino acid in the subject.

### BRIEF DESCRIPTION OF THE DRAWINGS

FIG. 1 depicts various branched chain amino acid degradative pathways and the metabolites and associated diseases relating to BCAA metabolism.

FIG. 2 depicts aspects of the branched chain amino acid degradative pathways for leucine, isoleucine, and valine.

FIG. 3 depicts aspects of alternate branched chain amino acid degradative pathways for leucine, isoleucine, and valine involving a ketoacid decarboxylase and an alcohol dehydrogenase, resulting in isopentanol, isobutanol, and 2-methylbutanol, respectively.

FIG. 4 depicts aspects of alternate branched chain amino acid degradative pathways for leucine, isoleucine, and valine involving a ketoacid decarboxylase and an aldehyde dehydrogenase, resulting in isovalerate, isobutyrate, and 2-methylbutyrate, respectively.

FIG. 5. depicts aspects of alternate branched chain amino acid degradative pathways for leucine, isoleucine, and valine involving a branched chain keto acid dehydrogenase complex (bkd), and the Liu operon from Pseudomonas aeruginosa, resulting in the acylCoA derivative of BCAA. In the case of leucine, the Liu operon coverts isovalerylCoA into acetoacetate and acetyl CoA.

FIG. 6 depicts the conversion of isovaleryCoA to acetoacetate and acetylCoA by the Liu operon enzymes. In the case of isovaleric acidemia, accumulating isovaleric acid can be activated into isovalerylCoA by an acylCoA synthetase, such as LbuL from Streptomyces lividans.

FIG. 7 depicts possible components of a branched chain amino acid synthetic biotic disclosed herein. An exemplary modified bacterium (E. Coli Nissle 1917) for metabolizing leucine to isopentanol may comprise gene sequence(s) for encoding one or more of the following: (1) livKHMGF (a high affinity leucine transporter that can transport leucine into the bacterial cell); (2) LivJHMGF (a high affinity BCAA transporter that can transport leucine, isoleucine, and valine into the bacterial cell); (3) leuDH (leucine dehydrogenase, e.g., derived from P. aeruginosa PA01 or Bacillus cereus which converts the BCAA into its corresponding α-ketoacid); (4) IlvE (branched chain amino acid aminotransferase, which also converts BCAA into its corresponding α-ketoacid); (5) KivD (branched chain α-ketoacid decarboxylase, e.g., derived from Lactococcus lactis IFPL730, which converts the α-ketoacid to its corresponding aldehyde); and (6) Adh2 (an alcohol dehydrogenase, e.g., derived from S. cerevisiae; which converts the aldehyde to its corresponding alcohol). The bacterium may further be a gene knockout for the gene encoding LeuE (leucine exporter; knocking out this gene keeps intracellular leucine concentration high) and/or the gene encoding IlvC (keto acid reductoisomerase, which is required for BCAA synthesis; knocking out this gene creates an auxotroph and requires the bacterial cell to import isoleucine and valine to survive).

FIG. 8 depicts possible components of a branched chain amino acid synthetic biotic disclosed herein. An exemplary modified bacterium for metabolizing leucine to isopentanol may comprise gene sequence(s) for encoding one or more of the following: (1) livKHMGF (a high affinity leucine transporter that can transport leucine into the bacterial cell); (2) BrnQ (a low affinity BCAA transporter that can transport branched chain amino acids into the bacterial cell); (3) leuDH (leucine dehydrogenase, e.g., derived from P. aeruginosa PA01 or Bacillus cereus, which converts the BCAA into its corresponding α-ketoacid); (4) IlvE (branched chain amino acid aminotransferase, which also converts BCAA into its corresponding α-ketoacid); (5) L-AAD (amino acid oxidase, which also converts BCAA into its corresponding α-ketoacid; LAAD(Pv)/LAAD(Pm) are from Proteus vulgaris and Proteus mirabilis, respectively); (6) KivD (branched chain α-ketoacid decarboxylase, e.g., derived from Lactococcus lactis IFPL730, which converts the α-ketoacid to its corresponding aldehyde); and (7) an alcohol dehydrogenase (e.g., Adh2, e.g., derived from S. cerevisiae; YghD, e.g., derived from E. coli, which converts the aldehyde to its corresponding alcohol). The bacterium may further be a gene knockout for the gene encoding LeuE (leucine exporter; knocking out this gene keeps intracellular leucine concentration high) and/or the gene encoding IlvC (keto acid reductoisomerase, which is required for BCAA synthesis; knocking out this gene creates an auxotroph and requires the bacterial cell to import isoleucine and valine to survive). An exemplary modified bacterium for metabolizing leucine to isovalerate may comprise gene sequence(s) for encoding one or more of the following: (1) livKHMGF (a high affinity leucine transporter that can transport leucine into the bacterial cell); (2) BrnQ (a low affinity BCAA transporter that can transport branched chain amino acids into the bacterial cell); (3) leudh (leucine dehydrogenase, e.g., derived from P. aeruginosa PA01 or Bacillus cereus, which converts the BCAA into its corresponding α-ketoacid); (4) IlvE (branched chain amino acid aminotransferase, which also converts BCAA into its corresponding α-ketoacid); (5) L-AAD (amino acid oxidase, which also converts BCAA into its corresponding α-ketoacid); (6) KivD (branched chain α-ketoacid decarboxylase, e.g., derived from Lactococcus lactis IFPL730, which converts the α-ketoacid to its corresponding aldehyde); and (7) an aldehyde dehydrogenase (e.g., PadA, e.g., derived from E. coli K12, which converts the aldehyde to its corresponding carboxylic acid). The bacterium may further be a gene knockout for the gene encoding LeuE (leucine exporter; knocking out this gene keeps intracellular leucine concentration high) and/or the gene encoding IlvC (keto acid reductoisomerase, which is required for BCAA synthesis; knocking out this gene creates an auxotroph and requires the bacterial cell to import isoleucine and valine to survive).

FIG. 9 depicts possible components of a branched chain amino acid synthetic biotic disclosed herein. An exemplary modified bacterium for metabolizing leucine to isopentanol is shown. Leucine is transported into the bacterium via the high affinity leucine transporter, LivKHMGF, where it is converted to alpha-ketoisocaproic acid using leuDH (Leucine dehydrogenase). The alpha-ketoisocaproic acid is converted to isovalderaldehyde using KivD (BCAA α-ketoacid decarboxylase) and further converted to isopentanol using Adh (alcohol dehydrogenase 2). One or more of the catabolic enzymes, transporters, or other genes may be under the control of an inducible promoter that is induced under exogenous environmental conditions, such as any of the inducible promoters provided herein, e.g., a promoter induced under low oxygen or anaerobic conditions.

FIG. 10 depicts one exemplary branched chain amino acid circuit. Genes shown are high affinity leucine transporter complex (LivKHMGF), leucine dehydrogenase (leuDH), e.g., from Pseudomonas aeruginosa, the branched chain α-ketoacid decarboxylase (KivD), e.g., from Lactococcus lactis, and alcohol dehydrogenase 2 (Adh2), e.g., from Saccharomyces cerevisiae, the genes for the leucine exporter (LeuE) and IlvC (keto acid reductoisomerase, required for BCAA synthesis) have been deleted. The gene for LivJ (a BCAA binding protein that can transport branched chain amino acids into the bacterial cell) is added which can be under the control of the native promoter or the constitutive promoter Ptac. One or more of the genes encoding a catabolic enzyme, transporter, and/or other genes (e.g., livJ) may be under the control of an inducible promoter that is induced under exogenous environmental conditions, such as any of the inducible promoters provided herein, e.g., a promoter induced under low oxygen or anaerobic conditions.

FIG. 11 depicts one exemplary branched chain amino acid circuit. Genes shown are high affinity leucine transporter complex (LivKHMGF), BCAA amino transferase (ilvE), the branched chain α-ketoacid decarboxylase (KivD), e.g., from Lactococcus lactis, and alcohol dehydrogenase 2 (Adh2), e.g., from Saccharomyces cerevisiae. The genes for the leucine exporter (LeuE) and keto acid reductoisomerase (IlvC) have been deleted. The gene for LivJ is added which can be under the control of the native promoter or the constitutive promoter Ptac. One or more of the genes encoding a catabolic enzyme, transporter, and/or other genes (e.g., livJ) may be under the control of an inducible promoter that is induced under exogenous environmental conditions, such as any of the inducible promoters provided herein, e.g., a promoter induced under low oxygen or anaerobic conditions.

FIG. 12 depicts one exemplary branched chain amino acid circuit. Genes shown are high affinity leucine transporter complex (LivKHMGF), BCAA amino transferase (ilvE), the branched chain α-ketoacid decarboxylase (KivD), e.g., from Lactococcus lactis, and alcohol dehydrogenase 2 (Adh2), e.g., from Saccharomyces cerevisiae. The genes for the leucine exporter (LeuE) and keto acid reductoisomerase (ilvC) have been deleted. The gene for LivJ is added which can be under the control of the native promoter or the constitutive promoter Ptac. One or more of the genes encoding a catabolic enzyme, transporter, and/or other genes (e.g., livJ) may be under the control of an inducible promoter that is induced under exogenous environmental conditions, such as any of the inducible promoters provided herein, e.g., a promoter induced under low oxygen or anaerobic conditions. In certain embodiments, any of the genes may be under the control of a tetR/tetA promoter. For example, the construct may comprise a (constitutive or inducible) promoter driving expression of the Tet repressor (TetR) from the tetR gene, which is linked to a second promoter comprising a TetR binding site that drives expression of any of the leucine import cassette(s) described above. TetR is (either constitutively or inducibly) expressed and inhibits the expression of the leucine import cassette(s). Upon addition of anhydrotetracylcine (ATC), TetR binds to ATC removing the inhibition by TetR allowing expression of the leucine import cassette(s).

FIGS. 13A-13F depict exemplary components of branched chain amino acid synthetic biotics. FIG. 13A and FIG. 13B depicts 2 exemplary components of a branched chain amino acid synthetic biotic disclosed herein for leucine catabolism to isopentanol or isovalerate (FIG. 13A) or alpha-ketoisocaproic acid (FIG. 13B), wherein the second step is catalyzed by Ketoacid decarboxylase (KivD). FIG. 13C depicts a schematic of the corresponding metabolic pathway for FIG. 13A and FIG. 13B. In some embodiments, both circuits can be expressed in the same strain. Alternatively, the circuits can each be expressed individually. Genes shown in FIGS. 13A and B are amino transferase (ilvE), leuDH (derived from P. aeruginosa PA01 or Bacillus cereus) and/or LAAD (derived from Proteus mirabilis or Proteus vulgaris) for conversion of BCAA to the α-keto acid; the branched chain α-ketoacid decarboxylase (KivD) for conversion from the α-keto acid to the corresponding aldehyde; and alcohol dehydrogenase 2 (Adh2; yqhD) for conversion to the corresponding alcohol or aldehyde dehydrogenase (padA) for conversion to the corresponding carboxylic acid. The genes for the leucine exporter (LeuE) and keto acid reductoisomerase (ilvC) can be deleted. FIG. 13D and FIG. 13E depict 2 exemplary components of a branched chain amino acid synthetic biotic disclosed herein for leucine catabolism to isovalerylCoA (FIG. 13E) or alpha-ketoisocaproic acid (FIG. 13E and FIG. 13F), wherein the second step is catalyzed by Bkd complex from Pseudomonas aeruginosa. FIG. 13F depicts a schematic of the corresponding metabolic pathway for FIG. 13D and FIG. 13E. In some embodiments, both circuits can be expressed in the same strain. Alternatively, the circuit shown in FIG. 13D can each be expressed individually, without the circuit of FIG. 13E. The circuit in FIG. 13E (the Liu operon) requires the circuit of FIG. 13D to generate its substrate, isovalerylCoA, and therefore is used together with the circuit of FIG. 13E. Genes shown in FIG. 13D and FIG. 13E are amino transferase (ilvE), leuDH (derived from P. aeruginosa PA01 or Bacillus cereus) and/or LAAD (derived from Proteus mirabilis or Proteus vulgaris) for conversion of BCAA to the α-keto acid; the Bkd complex (comprising bkdA1, bkdA2, bkdB, and lpdV) for conversion from the α-keto acid to the corresponding CoA thioester, and the Liu operon (comprising liuA, liuB, liuC, liuD, and liuE) for conversion of isovaleryl-CoA to acetoacetate and acetylCoA.

One or more of the genes encoding a catabolic enzyme, transporter, and/or other genes may be under the control of an inducible promoter that is induced under exogenous environmental conditions, such as any of the inducible promoters provided herein, e.g., a promoter induced under low oxygen or anaerobic conditions. In certain embodiments, the constructs are expressed on a high copy plasmid. In certain embodiments, any of the genes may be under the control of a tetR promoter. For example, the construct may comprise a (constitutive or inducible) promoter driving expression of the Tet repressor (TetR) from the tetR gene, which is linked to a second promoter comprising a TetR binding site that drives expression of any of the BCAA catabolic cassettes described above. TetR is (either constitutively or inducibly) expressed and inhibits the expression of the BCAA catabolic cassette(s). Upon addition of anhydrotetracylcine (ATC), TetR binds to ATC and binds removing the inhibition by TetR allowing expression of the BCAA catabolic cassettes.

FIGS. 14A and 14B depicts exemplary components of a branched chain amino acid synthetic biotic disclosed herein for leucine import. Genes shown are high affinity leucine transporter complex (LivKHMGF) (FIG. 14A) and low affinity BCAA transporter (brnQ) (FIG. 14B). One or more of the genes encoding a transporter may be under the control of an inducible promoter that is induced under exogenous environmental conditions, such as any of the inducible promoters provided herein, e.g., a promoter induced under low oxygen or anaerobic conditions. In certain embodiments, any of the genes may be under the control of a tetR promoter. In some embodiments, both circuits can be expressed in the same strain. Alternatively, the circuits can each be expressed individually. In some embodiments, the high affinity leucine transporter complex (LivKHMGF) and/or the low affinity BCAA transporter (brnQ) is integrated into the chromosome. Exemplary chromosomal insertion sites are shown in FIG. 68B, e.g., lacZ. In some embodiments, the high affinity leucine transporter complex (LivKHMGF) and/or the low affinity BCAA transporter (brnQ) is located on a plasmid, e.g., a low copy plasmid.

FIG. 15 depicts one exemplary branched chain amino acid circuit. Genes shown are high affinity leucine transporter complex (LivKHMGF), leuDH (e.g., derived from P. aeruginosa PA01 or Bacillus cereus), the branched chain α-ketoacid decarboxylase (KivD), e.g., from Lactococcus lactis, and alcohol dehydrogenase 2 (Adh2), e.g., from Saccharomyces cerevisiae. The genes for the leucine exporter (LeuE) and keto acid reductoisomerase (IlvC) have been deleted. The gene for LivJ is added which can be under the control of the native promoter or the constitutive promoter Ptac (a hybrid synthetic promoter derived from trp and lac). In certain embodiments, any of the genes may be under the control of a tetR/tetA promoter. For example, the construct may comprise a (constitutive or inducible) promoter driving expression of the Tet repressor (TetR) from the tetR gene, which is linked to a second promoter comprising a TetR binding site that drives expression of any of the leucine catabolic cassettes described above. TetR is (either constitutively or inducibly) expressed and inhibits the expression of the leucine catabolic cassette(s). Upon addition of anhydrotetracylcine (ATC), TetR binds to ATC and binds removing the inhibition by TetR allowing expression of the leucine catabolic cassettes.

FIG. 16 depicts one exemplary branched chain amino acid circuit. Genes shown are high affinity leucine transporter complex (LivKHMGF), leuDH (e.g., derived from Pseudomonas aeruginosa PA01 or Bacillus cereus), the branched chain α-ketoacid decarboxylase (KivD) e.g., from Lactococcus lactis, aldehyde dehydrogenase (PadA) e.g., from E. Coli K12. The genes for the leucine exporter (LeuE) and IlvC have been deleted. In certain embodiments, any of the genes may be under the control of a tetR/tetA promoter. For example, the construct may comprise a (constitutive or inducible) promoter driving expression of the Tet repressor (TetR) from the tetR gene, which is linked to a second promoter comprising a TetR binding site that drives expression of any of the leucine catabolic cassettes described above. TetR is (either constitutively or inducibly) expressed and inhibits the expression of the leucine catabolic cassette(s). Upon addition of anhydrotetracylcine (ATC), TetR binds to ATC and binds removing the inhibition by TetR allowing expression of the leucine catabolic cassettes.

FIG. 17 depicts one exemplary branched chain amino acid circuit. Genes shown are low affinity BCAA transporter (BrnQ), leuDH (e.g., derived from Pseudomonas aeruginosa PA01 or Bacillus cereus), the branched chain α-ketoacid decarboxylase (KivD), e.g., from Lactococcus lactis, aldehyde dehydrogenase (PadA), e.g., from E. Coli K-12. The genes for the leucine exporter (LeuE) and IlvC have been deleted. The gene for ilvE is added. In certain embodiments, any of the genes may be under the control of a tetR/tetA promoter. For example, the construct may comprise a (constitutive or inducible) promoter driving expression of the Tet repressor (TetR) from the tetR gene, which is linked to a second promoter comprising a TetR binding site that drives expression of any of the leucine catabolic cassettes described above. TetR is (either constitutively or inducibly) expressed and inhibits the expression of the leucine catabolic cassette(s). Upon addition of anhydrotetracylcine (ATC), TetR binds to ATC and binds removing the inhibition by TetR allowing expression of the leucine catabolic cassettes.

FIG. 18 depicts one exemplary branched chain amino acid circuit. Genes shown are high affinity leucine transporter complex (LivKHMGF), leuDH, e.g., derived from Pseudomonas aeruginosa PA01 or Bacillus cereus, the branched chain α-ketoacid decarboxylase (KivD), e.g., from Lactococcus lactis, and aldehyde dehydrogenase (PadA), e.g., from E. Coli K-12. The genes for the leucine exporter (LeuE) and IlvC have been deleted. The gene for BrnQ is added. In certain embodiments, any of the genes may be under the control of a tetR/tetA promoter. In some embodiments, the high affinity leucine transporter complex (LivKHMGF) and/or the low affinity BCAA transporter (brnQ) is integrated into the chromosome. Exemplary chromosomal insertion sites are shown in FIG. 68B, e.g., lacZ. In some embodiments, the transporters are expressed from a plasmid.

FIG. 19 depicts one exemplary branched chain amino acid circuit. Genes shown are high affinity leucine transporter complex (LivKHMGF), L-AAD, e.g., derived from Proteus vulgaris or Proteus mirabilis, the branched chain α-ketoacid decarboxylase (KivD), e.g., from Lactococcus lactis, and either aldehyde dehydrogenase (PadA), e.g., from E. Coli K-12, alcohol dehydrogenase YqhD, e.g., from E. coli, or alcohol dehydrogenase Adh2, e.g., from S. cerevisiae. The genes for the leucine exporter (LeuE) and IlvC have been deleted. The gene for BrnQ is added. In certain embodiments, any of the genes may be under the control of a tetR/tetA promoter.

FIG. 20 depicts one exemplary branched chain amino acid circuit. Genes shown are high affinity leucine transporter complex (LivKHMGF), L-AAD, e.g., derived from Proteus vulgaris or Proteus mirabilis, the branched chain α-ketoacid decarboxylase (KivD), e.g., from Lactococcus lactis, and either aldehyde dehydrogenase (PadA), e.g., from E. Coli K-12, alcohol dehydrogenase YqhD, e.g., from E. coli, or alcohol dehydrogenase Adh2 from S. cerevisiae. The genes for the leucine exporter (LeuE) and IlvC have been deleted. The gene for BrnQ is added. In some embodiments, any of the genes may be under the control of a promoter inducible under low oxygen or anaerobic conditions, e.g., an FNR promoter.

FIG. 21 depicts one exemplary branched chain amino acid circuit. Genes shown are high affinity leucine transporter complex (LivKHMGF), LeuDh, e.g., derived from Pseudomonas aeruginosa PA01 or Bacillus cereus, the branched chain α-ketoacid decarboxylase (KivD), e.g., from Lactococcus lactis, and either aldehyde dehydrogenase (PadA), e.g., from E. Coli K-12, alcohol dehydrogenase YqhD from E. coli, or alcohol dehydrogenase Adh2, e.g., from S. cerevisiae. The genes for the leucine exporter (LeuE) and IlvC have been deleted. The gene for BrnQ is added. In some embodiments, any of the genes may be under the control of a promoter inducible under low oxygen or anaerobic conditions, e.g., an FNR promoter.

FIG. 22 depicts one exemplary branched chain amino acid circuit. Genes shown are high affinity leucine transporter complex (LivKHMGF), low affinity BCAA transporter (brnQ), a leucine dehydrogenase leuDH (from Pseudomonas aeruginosa or Bacillus cereus), Bkd complex (comprising bkdA1, bkdA2, bkdB, and lpdV) for conversion from the α-keto acid to the corresponding CoA thioester. The genes for the leucine exporter (LeuE) and IlvC have been deleted. The gene for BrnQ is added. In some embodiments, any of the genes may be under the control of a promoter inducible under low oxygen or anaerobic conditions, e.g., an FNR promoter.

FIG. 23 depicts one exemplary branched chain amino acid circuit. Genes shown are high affinity leucine transporter complex (LivKHMGF), low affinity BCAA transporter (brnQ), a leucine dehydrogenase leuDH (from Pseudomonas aeruginosa or Bacillus cereus), the Bkd complex (comprising bkdA1, bkdA2, bkdB, and lpdV) for conversion from the α-keto acid to the corresponding CoA thioester, and Liu operon (comprising liuA, liuB, liuC, liuD, and liuE) for conversion of isovaleryl-CoA to acetoacetate and acetylCoA. The genes for the leucine exporter (LeuE) and IlvC have been deleted. The gene for BrnQ is added. In some embodiments, any of the genes may be under the control of a promoter inducible under low oxygen or anaerobic conditions, e.g., an FNR promoter.

FIG. 24 depicts one exemplary branched chain amino acid circuit.

FIGS. 25A, 25B, and 25C depict exemplary constructs of circuit components for LeuDH, kivD and livKHMGF inducible expression in E. coli. FIG. 25A depicts kivD under the control of the Tet promoter, e.g., cloned in a high-copy plasmid. FIG. 25B depicts kivD and LeuDH under the control of the Tet promoter, e.g., cloned into a high-copy plasmid. FIG. 25C depicts livKHMGF operon under the control of the Tet promoter, flanked by the lacZ homologous region for chromosomal integration by lamb-red recombination.

FIG. 26 depicts the gene organization of a Tet-kivD-adh2 construct.

FIG. 27 depicts the gene organization of a Tet-LeuDH-kivD-adh2 construct.

FIG. 28 depicts the gene organization of aTet-ilvE-kivD-adh2 construct.

FIG. 29 depicts the gene organization of the Tet-bkd operon construct.

FIG. 30 depicts the gene organization of the Tet-Leudh-bkd operon construct.

FIG. 31 depicts the gene organization of the Tet-livKHMGF construct.

FIG. 32 depicts the gene organization of the pKIKO-lacZ plasmid used to clone the Tet-livKHMGF construct.

FIG. 33 depicts the gene organization of the pTet-livKHMGF plasmid used to generate the PCR fragment used to integrate the Tet-livKHMGF into E. coli Nissle lacZ locus.

FIG. 34 depicts the gene organization of the DNA fragment used to generate the E. coli Nissle ΔleuE deletion strain.

FIG. 35 depicts the gene organization of the DNA fragment used to integrate the Tet-livKHMGF into the E. coli Nissle lacZ locus.

FIG. 36 depicts the organization of the DNA fragment used to exchange the endogenous livJ promoter with the constitutive promoter Ptac.

FIG. 37 depicts the gene organization of a LBUL construct.

FIG. 38 depicts leucine levels in the Nissle ΔleuE deletion strain harboring a high-copy plasmid expressing kivD from the Tet promoter or further with a copy of the livKHMGF operon driven by the Tet promoter integrated into the chromosome at the lacZ locus, which were induced with ATC and incubated in culture medium supplemented with 2 mM leucine. Samples were removed at 0, 1.5, 6 and 18 h, and leucine concentration was determined by liquid chromatography tandem mass spectrometry.

FIG. 39 depicts leucine degradation in the Nissle ΔleuE deletion strain harboring a high-copy plasmid expressing the branch-chain keto-acid dehydrogenase (bkd) complex (comprising bkdA1, bkdA2, bkdB, and lpdV) with or without expression of a leucine dehydrogenase (LeuDH) from the Tet promoter or further with a copy of the leucine importer livKHMGF driven by the Tet promoter integrated into the chromosome at the lacZ locus, which were induced with ATC and incubated in culture medium supplemented with 2 mM leucine. Samples were removed at 0, 1.5, 6 and 18 h, and leucine concentration was determined by liquid chromatography tandem mass spectrometry.

FIGS. 40A, 40B, and 40C depict the simultaneous degradation of leucine (FIG. 40A), isoleucine (FIG. 40B), and valine (FIG. 40C) by E. coli Nissle and its ΔleuE deletion strain harboring a high-copy plasmid expressing the keto-acid decarboxylase kivD from the Tet promoter or further with a copy of the livKHMGF operon driven by the Tet promoter integrated into the chromosome at the lacZ locus, which were induced with ATC and incubated in culture medium supplemented with 2 mM leucine, 2 mM isoleucine and 2 mM valine. Samples were removed at 0, 1.5, 6 and 18 h, and BCAA concentration was determined by liquid chromatography tandem mass spectrometry. The strains were grown overnight at 37° C. in LB media, and the overnight culture was used to inoculate a new batch at a 1/100 dilution in LB, which was grown for three hours at 37° C. Induction was for two hours with 100 ng/mL ATC. The cells were then collected by centrifugation and resuspended in M9+0.5% glucose and 2 mM each of leucine, isoleucine, and valine. Samples were removed at 0, 1.5, 6 and 18 h, and BCAA concentration was determined by liquid chromatography tandem mass spectrometry. The results demonstrate that isoleucine and valine were also consumed by leucine-degrading strains. Moreover, deletion of leuE and expression of livKHMGF improved the rate of BCAA degradation.

FIG. 41 depicts a bar graph showing that the expression of kivD in E. coli Nissle leads to leucine degradation in vitro. The strains were grown overnight at 37° C. in LB media, and the overnight culture was used to inoculate a new batch at a 1/100 dilution in LB. Induction was for two hours with 100 ng/mL ATC. The cells were then collected by centrifugation and resuspended in M9+0.5% glucose and 2 mM leucine. Aliquots were removed at the indicated times for leucine determination by mass spectrometry. Inclusion of kivD resulted in increased bacterial cell consumption of leucine.

FIGS. 42A and 42B depict the determination of the leucine degradation rate, as mediated by KivD. The strains were grown overnight at 37° C. in LB media, and the overnight culture was used to inoculate a new batch at a 1/100 dilution in LB, which was grown for two hours at 37° C. Induction was for one hour with 100 ng/mL ATC. The cells were then collected by centrifugation and resuspended in M9+0.5% glucose and 2 mM leucine at OD₆₀₀=1. Samples were collected at 3 hours. The total degradation rate was about 250 nmol/10⁹ CFU/hour. The degradation rate attributable to KivD was about 50 nmol/10⁹ CFU/hour.

FIGS. 43A, 43B, and 43C depict bar graphs which show the efficient degradation of leucine (FIG. 43A), isoleucine (FIG. 43B), and valine (FIG. 43C) by the engineered strains. FIG. 43D depicts a bar graph showing that expression of leucine dehydrogenase (LeuDH from Pseudomonas aeruginosa) improves the rate of leucine degradation to about 160 nmol/10⁹ CFU/hour. The background strain is Nissle ΔleuE, lacZ:tet-livKHMGF.

FIG. 44 depicts the pathway of leucine degradation and KIC degradation engineered into the SYN469 strain.

FIGS. 45A and 45B depict the rate of leucine degradation or KIC degradation in several different engineered bacteria. The background strain used was SYN469 (ΔleuEΔilvC,lacZ::tet-livKHMGF), and the circuit was under the control of the Tet promoter on a high-copy plasmid. SYN479, SYN467, SYN949, SYN954, and SYN950 strains were fed leucine (FIG. 45A) or ketoisocaproate (MC, also known as 4-methyl-2-oxopentanoate) (FIG. 45B), and products were monitored. A higher conversion of MC than leucine to end-products demonstrates that leucine uptake and/or conversion to MC is rate-limiting.

FIG. 46 depicts the use of valine sensitivity in E. coli as a genetic screening tool. There are three AHAS (acetohydroxybutanoate synthase) isozymes in E. coli (AHAS I: ilvBN, AHAS II: ilvGM, and AHAS III: ilvIH). Valine and leucine exert feedback inhibition on AHAS I and AHAS III; AHAS II is resistant to Val and Leu inhibition. E. coli K12 has a frameshift mutation in ilvG (AHAS II) and is unable to produce isoleucine and leucine in the presence of valine. Nissle has a functional ilvG and is insensitive to valine and leucine. A genetically engineered strain derived from E. coli K12, which more efficiently degrades leucine, has a greater reduction in sensitivity to leucine (through relieving the feedback inhibition on AHAS I and III). As a result, this pathway can be used as a tool to select and identify a strain with improved resistance to leucine.

FIG. 47A depicts a bar graph showing the leucine degradation rates for various engineered bacterial strains. Bacterial strain SYN469 is a leuE and ilvC knockout and comprises the leucine transporter under the control of tet promoter. Other tested engineered bacterial strains include: (1) strain having ilvE, kivD, and adh2; (2) strain having leuDh, kivD, and adh2; and (3) strain having L-AAD, kivD, and adh2. The strains are tet-inducible constructs on a high copy plasmid. The results show that L-amino acid deaminase (L-AAD) provides the best leucine degradation rate. FIG. 47B depicts a schematic of the corresponding pathways.

FIG. 48A shows the leucine degradation rates for various engineered bacterial strains. Bacterial strain SYN469 is a leuE and ilvC knockout and comprises the leucine transporter under the control of tet promoter. Other tested engineered bacterial strains include: (1) strain having L-AAD derived from P. vulgaris, kivD, and adh2; (2) strain having L-AAD derived from P. vulgaris (LAAD_{Pv}), kivD, and yqhD; (3) strain having L-AAD derived from P. vulgaris, kivD, and padA and (4) strain having L-AAD derived from P. mirabilis (LAAD_{Pm}). The results show that yqhD, adh2, and padA have similar activities and that LAAD_{Pm} is a good alternative to LAAD_{PV}. FIG. 48B depicts a schematic of the corresponding pathways.

FIG. 49A shows the leucine degradation rates for various engineered bacterial strains. Bacterial strain SYN458 is a leuE knockout. SYN452 is a leuE knockout and comprises the leucine transporter under the control of tet promoter. These background strains were tested with bacterial strains additionally having leuDH derived from P. aeruginosa, kivD, and padA. The results show that overexpression of the high affinity leucine transporter livKHMGF does not dramatically improved the rate of leucine degradation in a LeuE knockout strain having LeuDH, kivD, and padA with and without the leucine transporter livKHMGF under the control of tet promoter as measured by leucine degradation, KIC production, and isovalerate production. FIG. 49B depicts a schematic of the corresponding pathways.

FIGS. 50A and 50B depict a bar graph which shows the leucine degradation rates for various engineered bacterial strains. SYN469 is a LeuE and ilvC knockout bacterial strain and comprises the leucine transporter under the control of a tet promoter. The tet inducible leuDH-kivD-padA construct was expressed on a high copy plasmid. Two different leucine dehydrogenases were used in the tested constructs: leuDH_{PA} derived from P. aeruginosa and leuDH_{BC} derived from Bacillus cereus. The tet inducible brnQ construct was expressed on a low copy plasmid. FIG. 50A depicts a bar graph which shows that overexpression of the low-affinity BCAA transporter BrnQ greatly improves the rate of leucine degradation in a LeuE and ilvC knockout bacterial strain having either LeuDH derived from P. aeruginosa or LeuDH derived from Bacillus cereus, kivD, and padA with and without the BCAA transporter brnQ under the control of tet promoter as measured by leucine degradation, KIC production, and isovalerate production. FIG. 50B depicts a bar graph which shows the overexpression of the low-affinity BCAA transporter BrnQ greatly improves the rate of leucine degradation in leuDH-kivD-padA constructs. FIG. 50C depicts a schematic of the corresponding pathways.

FIG. 51 depicts a screening strategy used to identify bacterial mutants with increased Leucine transport into the bacterial cell using a leucine auxotroph. L-leucine is replaced with D-Leucine in the media. The bacteria can grow in the presence of D-leucine, because the bacterial stain has a racemase, which can convert D-leucine to L-leucine. However, the uptake of D-leucine through LivKHMGF is less efficient than the uptake of L-leucine. The leucine auxotroph can still grow if high concentrations of D-Leucine are provided, even though the D-leucine uptake is less efficient than L-leucine uptake. When concentrations of D-leucine in the media are lowered, the cells can no longer grow, unless transport efficiency is increased, ergo mutants with increased D-leucine uptake can be selected.

FIG. 52 depicts a graph which shows that leucine is able to recirculate from the periphery into the small intestine. BL6 animals were subjected to subcutaneous injection of isotopic leucine (¹³C₆) (0.1 mg/g). Plasma, small intestine (SI), large intestine (LI) and cecum effluent was tested for the presence of ¹³C₆-Leucine. This experiment can be repeated in iMSUD animals (−/− or −/+).

FIG. 53 depicts a bar graph showing the efficient import of valine by the expression of an inducible leucine high affinity transporter, livKHMGF, and the constitutive expression of livJ encoding for the BCAA binding protein of the BCAA high affinity transporter livJHMHGF. The natural secretion of valine by E. coli Nissle is observed for the ΔleuE strain. The secretion of valine is strongly reduced for ΔleuE, lacZ:Ptet-livKHMGF in the presence of ATC. This strongly suggests that the secreted valine is efficiently imported back into the cell by livKHMGF. The secretion of valine is abolished in the ΔleuE, lacZ:Ptet-livKHMGF, Ptac-livJ strain, with or without ATC. This strongly suggests that the constitutive expression of livJ is sufficient to import back the entire amount of valine secreted by the cell via the livJHMGF transporter.

FIG. 54A and FIG. 54B depict bar graphs of leucine concentrations (FIG. 54A) and degradation rates (FIG. 54B) measured in an in vitro leucine degradation assay comparing strains with (SYN1980) and without (SYN1992) a tetracycline inducible brnQ construct. FIG. 54A depicts a bar graph of leucine concentrations present at 0, 1.5 and 3 h in the media of SYN1992 (ΔleuE, ΔilvC, lacZ:tetR-Ptet-livKHMGF, tetR-Ptet-leuDH(Bc)-kivD-adh2-rrnB ter (pSC101)) and SYN1980 (ΔleuE, ΔilvC, lacZ:tetR-Ptet-livKHMGF, tetR-Ptet-leuDH(Bc)-kivD-adh2-brnQ-rrnB ter (pSC101)). SYN469 (comprising ΔleuE, ΔilvC, and integrated lacZ:tetR-Ptet-livKHMGF) was used as a control. FIG. 54B depicts a bar graph showing the leucine degradation rates for SYN1992, SYN1980, and SYN469 in the presence and absence of ATC. Leucine degradation rates were increased in both SYN1992 and SYN1980 upon addition of tetracycline, with SYN1980 (comprising tet-inducible BrnqQ) having a greater overall degradation rate. FIG. 54C depicts a schematic of a construct comprising codon optimized LeuDH-kivD-adh2-brnQ construct driven by a tetracycline inducible promoter, e.g., as used in FIG. 54A and FIG. 54B. FIG. 54D depicts a schematic of a construct comprising codon optimized LeuDH-kivD-padA-brnQ construct driven by a tetracycline inducible promoter; in other embodiments, the construct can be driven by a different promoter, e.g., an FNR promoter. FIG. 54E depicts a schematic of a construct comprising codon optimized LeuDH-kivD-yqhD-brnQ construct driven by a tetracycline inducible promoter; in other embodiments, the construct can be driven by a different promoter, e.g., an FNR promoter.

FIG. 55A and FIG. 55B depict bar graphs of leucine concentrations (FIG. 55A) and degradation rates (FIG. 55B) measured in an in vitro leucine degradation assay comparing strains with (SYN1981) and without (SYN1993) an anaerobic inducible brnQ construct. FIG. 55A depicts a bar graph of leucine concentrations present at 0, 1.5 and 3 h in the media of SYN1993 (ΔleuE, ΔilvC, lacZ:tetR-Ptet-livKHMGF, PfnrS-leuDH(Bc)-kivD-adh2-rrnB ter (pSC101)) and SYN1981 (ΔleuE, ΔilvC, lacZ:tetR-Ptet-livKHMGF, PfnrS-leuDH(Bc)-kivD-adh2-brnQ-rrnB ter (pSC101)). SYN469 (comprising ΔleuE, ΔilvC, and integrated lacZ:tetR-Ptet-livKHMGF) was used as a control. FIG. 55B depicts a bar graph showing the leucine degradation rates for SYN1993, SYN1981, and SYN469 with or without anaerobic induction of FNR mediated expression. Leucine degradation rates were increased in both SYN1993 and SYN1981 upon anaerobic induction, with SYN1981 (comprising FNR-inducible BrnQ) having a greater overall degradation rate. FIG. 55C depicts a schematic of a construct comprising codon optimized LeuDH-kivD-adh2-brnQ construct driven by an FNR promoter, e.g., as used in FIG. 55A and FIG. 55B.

FIG. 56A, FIG. 56B, FIG. 56C, FIG. 56D, FIG. 56E, FIG. 56F, FIG. 56G, FIG. 56H, FIG. 56I depict graphs showing the ability of engineered strain SYN1980 (comprising ΔleuE, ΔilvC, lacZ:tetR-Ptet-livKHMGF, tetR-Ptet-leuDH(Bc)-kivD-adh2-brnQ-rrnB ter (in low-copy pSC101 plasmid) to decrease plasma BCAA levels in vivo in the intermediate MSUD (iMSUD) animal model. SYN1980 was compared to wild type Nissle with a streptomycin resistance in this study. FIG. 56A, FIG. 56B, and FIG. 56C show plasma leucine, valine and isoleucine concentrations on day 1 and day 3 of the study. FIG. 56D, FIG. 56E, and FIG. 56F show the changes in leucine, valine and isoleucine concentrations observed in plasma. FIG. 56G, FIG. 56H, and FIG. 56I show the changes in leucine, valine and isoleucine concentrations observed in the brain. Levels of Leu and Val remained lower in the plasma of SYN1980-treated animals, resulting in a lower ΔLeu and ΔVal (FIG. 56A, FIG. 56B, FIG. 56D, FIG. 56E), as compared to animals treated with streptomycin resistant Nissle or vehicle control, where the switch to high protein diet lead to increased levels Leu and Val. Similar trend of lower Leu and Val and reduced ΔLeu and ΔVal was found in the brain (FIG. 56G, FIG. 56H). No significant changes in Ile concentrations in plasma or brain were observed; the switch to high protein chow did not seem to increase Ile levels (FIG. 56C, FIG. 56F, and FIG. 56I), consistent with the observations in Zinnanti et al for the iMSUD model.

FIG. 57 depicts a graph showing the scoring of videos for the number of ambulations of iMSUD mice switched to high protein chow and either gavaged with the BCAA consuming strain SYN1980 or with streptomycin resistant wild type Nissle on day one and three after the switch to high protein chow. The surviving mouse gavaged with SYN1980 showed reduced activity on day 3 as compared to day 1, but significantly greater activity than mice gavaged with streptomycin resistant E. coli Nissle. A second mouse gavaged with SYN1980 died of unrelated causes during the study procedure.

FIG. 58A and FIG. 58B depict schematics of the states of non-limiting embodiments of the disclosure. FIG. 58A depicts a schematic of the state of exemplary kivD and livKHMGF constructs under non-inducing conditions, and relatively low KivD and LivKHMGF production under aerobic conditions due to oxygen (O2) preventing FNR from dimerizing and activating the FNR responsive promoter and the kivD or livKHMGF genes under its control. FIG. 58B depicts a schematic of the state of one non-limiting embodiment of the kivD or livKHMGF construct under inducing (low oxygen or anaerobic) conditions. FIG. 58B depicts up-regulated KivD and LivHKMGF production under anaerobic conditions due to FNR dimerizing and inducing FNR responsive promoter-mediated expression of kivD and livKHMGF (squiggle above kivD and livKHMGF). Each arrow adjacent to one or a cluster of rectangles depicts the promoter responsible for driving transcription, in the direction of the arrow, of such gene(s). Arrows above each rectangle depict the expression product of each gene.

FIG. 59A and FIG. 59B depict schematics of non-limiting embodiments of the disclosure. FIG. 59A depicts a schematic of one exemplary branched chain amino acid circuit and an exemplary of kill switch design combined in one strain. Genes shown are high affinity leucine transporter complex (LivKHMGF), BCAA amino transferase (ilvE), the branched chain α-ketoacid decarboxylase (KivD), e.g., from Lactococcus lactis, and alcohol dehydrogenase 2 (Adh2), e.g., from Saccharomyces cerevisiae. The genes for the leucine exporter (LeuE) and keto acid reductoisomerase (IlvC) have been deleted. The gene for LivJ is added which can be under the control of the native promoter or the constitutive promoter Ptac. One or more of the genes encoding a catabolic enzyme, transporter, and/or other genes (e.g., livJ) may be under the control of an inducible promoter that is induced under exogenous environmental conditions, such as any of the inducible promoters provided herein, e.g., a promoter induced under low oxygen or anaerobic conditions. The strain also comprises a repression-based kill switch in which the AraC transcription factor is activated in the presence of arabinose and induces expression of TetR and an anti-toxin. TetR prevents the expression of the toxin. When arabinose is removed, TetR and the anti-toxin do not get made and the toxin is produced which kills the cell. FIG. 59B depicts a schematic of one exemplary branched chain amino acid circuit and a ThyA auxotrophy. Genes shown are high affinity leucine transporter complex (LivKHMGF), BCAA amino transferase (ilvE), the branched chain α-ketoacid decarboxylase (KivD), e.g., from Lactococcus lactis, and alcohol dehydrogenase 2 (Adh2), e.g., from Saccharomyces cerevisiae. The genes for the leucine exporter (LeuE) and keto acid reductoisomerase (IlvC) have been deleted. The gene for LivJ is added which can be under the control of the native promoter or the constitutive promoter Ptac. One or more of the genes encoding a catabolic enzyme, transporter, and/or other genes (e.g., livJ) may be under the control of an inducible promoter that is induced under exogenous environmental conditions, such as any of the inducible promoters provided herein, e.g., a promoter induced under low oxygen or anaerobic conditions.

FIG. 60A, FIG. 60B, FIG. 60C and FIG. 60D depict schematics of non-limiting examples of embodiments of the disclosure. FIG. 60A depicts a schematic of a non-limiting embodiment of the disclosure, wherein the expression of a heterologous gene is activated by an exogenous environmental signal, e.g., low-oxygen conditions. In the absence of arabinose, the AraC transcription factor adopts a conformation that represses transcription. In the presence of arabinose, the AraC transcription factor undergoes a conformational change that allows it to bind to and activate the araBAD promoter, which induces expression of TetR (tet repressor) and an anti-toxin. The anti-toxin builds up in the recombinant bacterial cell, while TetR prevents expression of a toxin (which is under the control of a promoter having a TetR binding site). However, when arabinose is not present, both the anti-toxin and TetR are not expressed. Since TetR is not present to repress expression of the toxin, the toxin is expressed and kills the cell. FIG. 60A also depicts another non-limiting embodiment of the disclosure, wherein the expression of an essential gene not found in the recombinant bacteria is activated by an exogenous environmental signal. In the absence of arabinose, the AraC transcription factor adopts a conformation that represses transcription of the essential gene under the control of the araBAD promoter and the bacterial cell cannot survive. In the presence of arabinose, the AraC transcription factor undergoes a conformational change that allows it to bind to and activate the araBAD promoter, which induces expression of the essential gene and maintains viability of the bacterial cell. FIG. 60B depicts a schematic of a non-limiting embodiment of the disclosure, where an anti-toxin is expressed from a constitutive promoter, and expression of a heterologous gene is activated by an exogenous environmental signal. In the absence of arabinose, the AraC transcription factor adopts a conformation that represses transcription. In the presence of arabinose, the AraC transcription factor undergoes a conformational change that allows it to bind to and activate the araBAD promoter, which induces expression of TetR, thus preventing expression of a toxin. However, when arabinose is not present, TetR is not expressed, and the toxin is expressed, eventually overcoming the anti-toxin and killing the cell. The constitutive promoter regulating expression of the anti-toxin should be a weaker promoter than the promoter driving expression of the toxin. The araC gene is under the control of a constitutive promoter in this circuit. FIG. 60C depicts a schematic of a repression-based kill switch in which the AraC transcription factor is activated in the presence of arabinose and induces expression of TetR and an anti-toxin. TetR prevents the expression of the toxin. When arabinose is removed, TetR and the anti-toxin do not get made and the toxin is produced which kills the cell. FIG. 60D depicts another non-limiting embodiment of the disclosure, wherein the expression of a heterologous gene is activated by an exogenous environmental signal. In the absence of arabinose, the AraC transcription factor adopts a conformation that represses transcription. In the presence of arabinose, the AraC transcription factor undergoes a conformational change that allows it to bind to and activate the araBAD promoter, which induces expression of TetR (tet repressor) and an anti-toxin. The anti-toxin builds up in the recombinant bacterial cell, while TetR prevents expression of a toxin (which is under the control of a promoter having a TetR binding site). However, when arabinose is not present, both the anti-toxin and TetR are not expressed. Since TetR is not present to repress expression of the toxin, the toxin is expressed and kills the cell. The araC gene is under the control of a constitutive promoter in this circuit.

FIG. 61 depicts a schematic of one non-limiting embodiment of the disclosure, where an exogenous environmental condition, e.g., low-oxygen conditions, or one or more environmental signals activates expression of a heterologous gene and at least one recombinase from an inducible promoter or inducible promoters. The recombinase then flips a toxin gene into an activated conformation, and the natural kinetics of the recombinase create a time delay in expression of the toxin, allowing the heterologous gene to be fully expressed. Once the toxin is expressed, it kills the cell.

FIG. 62 depicts a schematic of another non-limiting embodiment of the disclosure, where an exogenous environmental condition, e.g., low-oxygen conditions, or one or more environmental signals activates expression of a heterologous gene, an anti-toxin, and at least one recombinase from an inducible promoter or inducible promoters. The recombinase then flips a toxin gene into an activated conformation, but the presence of the accumulated anti-toxin suppresses the activity of the toxin. Once the exogenous environmental condition or cue(s) is no longer present, expression of the anti-toxin is turned off. The toxin is constitutively expressed, continues to accumulate, and kills the bacterial cell.

FIG. 63 depicts a schematic of one non-limiting embodiment of the disclosure, in which the genetically engineered bacteria produces equal amount of a Hok toxin and a short-lived Sok anti-toxin. When the cell loses the plasmid, the anti-toxin decays, and the cell dies. In the upper panel, the cell produces equal amounts of toxin and anti-toxin and is stable. In the center panel, the cell loses the plasmid and anti-toxin begins to decay. In the lower panel, the anti-toxin decays completely, and the cell dies.

FIG. 64 depicts a schematic of another non-limiting embodiment of the disclosure, where an exogenous environmental condition, e.g., low-oxygen conditions, or one or more environmental signals activates expression of a heterologous gene and at least one recombinase from an inducible promoter or inducible promoters. The recombinase then flips at least one excision enzyme into an activated conformation. The at least one excision enzyme then excises one or more essential genes, leading to senescence, and eventual cell death. The natural kinetics of the recombinase and excision genes cause a time delay, the kinetics of which can be altered and optimized depending on the number and choice of essential genes to be excised, allowing cell death to occur within a matter of hours or days. The presence of multiple nested recombinases can be used to further control the timing of cell death.

FIG. 65 depicts a schematic of another non-limiting embodiment of the disclosure, where an exogenous environmental condition, e.g., low-oxygen conditions, or one or more environmental signals activates expression of a heterologous gene, an anti-toxin, and at least one recombinase from an inducible promoter or inducible promoters. The recombinase then flips a toxin gene into an activated conformation, but the presence of the accumulated anti-toxin suppresses the activity of the toxin. Once the exogenous environmental condition or cue(s) is no longer present, expression of the anti-toxin is turned off. The toxin is constitutively expressed, continues to accumulate, and kills the bacterial cell.

FIG. 66 depicts an example of a genetically engineered bacteria that comprises a plasmid that has been modified to create a host-plasmid mutual dependency, such as the GeneGuard system described in more detail herein.

FIG. 67A, FIG. 67B, FIG. 67C, and FIG. 67D depict schematics of non-limiting examples of the gene organization of plasmids, which function as a component of a biosafety system (FIG. 67A and FIG. 67B), which also contains a chromosomal component (shown in FIG. 67C and FIG. 67D). The Biosafety Plasmid System Vector comprises Kid Toxin and R6K minimal ori, dapA (FIG. 67A) and thyA (FIG. 67B) and promoter elements driving expression of these components. In a non-limiting example, the plasmid comprises SEQ ID NO: 81. In a non-limiting example, the plasmid comprises SEQ ID NO: 82. In some embodiments, bla is knocked out and replaced with one or more constructs described herein, in which PAL3 and/or PheP and/or LAAD are expressed from an inducible or constitutive promoter. FIG. 67C and FIG. 67D depict schematics of the gene organization of the chromosomal component of a biosafety system. FIG. 67C depicts a construct comprising low copy Rep (Pi) and Kis antitoxin, in which transcription of Pi (Rep), which is required for the replication of the plasmid component of the system, is driven by a low copy RBS containing promoter. In some embodiments, the construct comprises SEQ ID NO: 89. FIG. 67D depicts a construct comprising a medium-copy Rep (Pi) and Kis antitoxin, in which transcription of Pi (Rep), which is required for the replication of the plasmid component of the system, is driven by a medium copy RBS containing promoter. In some embodiments, the construct comprises SEQ ID NO: 90. If the plasmid containing the functional DapA is used (as shown in FIG. 67A), then the chromosomal constructs shown in FIG. 67C and FIG. 67D are knocked into the DapA locus. If the plasmid containing the functional ThyA is used (as shown in FIG. 67B), then the chromosomal constructs shown in FIG. 67C and FIG. 67D are knocked into the ThyA locus. In this system, the bacteria comprising the chromosomal construct and a knocked out dapA or thyA gene can grow in the absence of dap or thymidine only in the presence of the plasmid.

FIG. 68A depicts an exemplary schematic of the E. coli 1917 Nissle chromosome comprising multiple mechanisms of action (MoAs). FIG. 68B depicts a map of exemplary integration sites within the E. coli 1917 Nissle chromosome. These sites indicate regions where circuit components may be inserted into the chromosome without interfering with essential gene expression. Backslashes (/) are used to show that the insertion will occur between divergently or convergently expressed genes. Insertions within biosynthetic genes, such as thyA, can be useful for creating nutrient auxotrophies. In some embodiments, an individual circuit component is inserted into more than one of the indicated sites.

FIG. 69 depicts three bacterial strains which constitutively express red fluorescent protein (RFP). In strains 1-3, the rfp gene has been inserted into different sites within the bacterial chromosome, and results in varying degrees of brightness under fluorescent light. Unmodified E. coli Nissle (strain 4) is non-fluorescent.

FIG. 70 depicts a graph of Nissle residence in vivo. Streptomycin-resistant Nissle was administered to mice via oral gavage without antibiotic pre-treatment. Fecal pellets from six total mice were monitored post-administration to determine the amount of administered Nissle still residing within the mouse gastrointestinal tract. The bars represent the number of bacteria administered to the mice. The line represents the number of Nissle recovered from the fecal samples each day for 10 consecutive days.

FIG. 71 depicts a bar graph of residence over time for streptomycin resistant Nissle in various compartments of the intestinal tract at 1, 4, 8, 12, 24, and 30 hours post gavage. Mice were treated with approximately 10⁹ CFU, and at each timepoint, animals (n=4) were euthanized, and intestine, cecum, and colon were removed. The small intestine was cut into three sections, and the large intestine and colon each into two sections. Intestinal effluents gathered and CFUs in each compartment were determined by serial dilution plating.

FIG. 72 depicts a schematic of a secretion system based on the flagellar type III secretion in which an incomplete flagellum is used to secrete a therapeutic peptide of interest (star) by recombinantly fusing the peptide to an N-terminal flagellar secretion signal of a native flagellar component so that the intracellularly expressed chimeric peptide can be mobilized across the inner and outer membranes into the surrounding host environment.

FIG. 73 depicts a schematic of a type V secretion system for the extracellular production of recombinant proteins in which a therapeutic peptide (star) can be fused to an N-terminal secretion signal, a linker and the beta-domain of an autotransporter. In this system, the N-terminal signal sequence directs the protein to the SecA-YEG machinery which moves the protein across the inner membrane into the periplasm, followed by subsequent cleavage of the signal sequence. The beta-domain is recruited to the Bam complex where the beta-domain is folded and inserted into the outer membrane as a beta-barrel structure. The therapeutic peptide is then thread through the hollow pore of the beta-barrel structure ahead of the linker sequence. The therapeutic peptide is freed from the linker system by an autocatalytic cleavage or by targeting of a membrane-associated peptidase (scissors) to a complementary protease cut site in the linker.

FIG. 74 depicts a schematic of a type I secretion system, which translocates a passenger peptide directly from the cytoplasm to the extracellular space using HlyB (an ATP-binding cassette transporter); HlyD (a membrane fusion protein); and TolC (an outer membrane protein) which form a channel through both the inner and outer membranes. The secretion signal-containing C-terminal portion of HlyA is fused to the C-terminal portion of a therapeutic peptide (star) to mediate secretion of this peptide.

FIG. 75 depicts a schematic of the outer and inner membranes of a gram-negative bacterium, and several deletion targets for generating a leaky or destabilized outer membrane, thereby facilitating the translocation of a therapeutic polypeptides to the extracellular space, e.g., therapeutic polypeptides of eukaryotic origin containing disulphide bonds. Deactivating mutations of one or more genes encoding a protein that tethers the outer membrane to the peptidoglycan skeleton, e.g., lpp, ompC, ompA, ompF, tolA, tolB, pal, and/or one or more genes encoding a periplasmic protease, e.g., degS, degP, nlpI, generates a leaky phenotype. Combinations of mutations may synergistically enhance the leaky phenotype.

FIG. 76 depicts a modified type 3 secretion system (T3SS) to allow the bacteria to inject secreted therapeutic proteins into the gut lumen. An inducible promoter (small arrow, top), e.g. a FNR-inducible promoter, drives expression of the T3 secretion system gene cassette (3 large arrows, top) that produces the apparatus that secretes tagged peptides out of the cell. An inducible promoter (small arrow, bottom), e.g. a FNR-inducible promoter, drives expression of a regulatory factor, e.g. T7 polymerase, that then activates the expression of the tagged therapeutic peptide (hexagons).

FIG. 77A, FIG. 77B, and FIG. 77C depict schematics of the gene organization of exemplary circuits of the disclosure for the expression of therapeutic polypeptides, which are secreted using components of the flagellar type III secretion system. A therapeutic polypeptide of interest, such as a BCAA catabolism enzyme, is assembled behind a fliC-5′UTR, and is driven by the native fliC and/or fliD promoter (FIG. 77A and FIG. 77B) or a Tet-inducible promoter (FIG. 77C). In alternate embodiments, an inducible promoter such as oxygen level-dependent promoters (e.g., FNR-inducible promoter), and promoters induced by a metabolite that may or may not be naturally present (e.g., can be exogenously added) in the gut, e.g., arabinose can be used. The therapeutic polypeptide of interest is either expressed from a plasmid (e.g., a medium copy plasmid) or integrated into fliC loci (thereby deleting all or a portion of fliC and/or fliD). Optionally, an N terminal part of FliC is included in the construct, as shown in FIG. 77B and FIG. 77C.

FIG. 78A and FIG. 78B depict schematics of the gene organization of exemplary circuits of the disclosure for the expression of therapeutic polypeptides, which are secreted via a diffusible outer membrane (DOM) system. The therapeutic polypeptide of interest, e.g., a BCAA catabolism enzyme, is fused to a prototypical N-terminal Sec-dependent secretion signal or Tat-dependent secretion signal, which is cleaved upon secretion into the periplasmic space. Exemplary secretion tags include sec-dependent PhoA, OmpF, OmpA, cvaC, and Tat-dependent tags (TorA, FdnG, DmsA). In certain embodiments, the genetically engineered bacteria comprise deletions in one or more of lpp, pal, tolA, and/or nlpI. Optionally, periplasmic proteases are also deleted, including, but not limited to, degP and ompT, e.g., to increase stability of the polypeptide in the periplasm. A FRT-KanR-FRT cassette is used for downstream integration. Expression is driven by a Tet promoter (FIG. 78A) or an inducible promoter, such as oxygen level-dependent promoters (e.g., FNR-inducible promoter, FIG. 78B), and promoters induced by a metabolite that may or may not be naturally present (e.g., can be exogenously added) in the gut, e.g., arabinose.

FIG. 79A depicts a “Oxygen bypass switch” useful for aerobic pre-induction of a strain comprising one or proteins of interest (POI), e.g., one or more BCAA catabolism enzyme(s) (POI1) and/or one or more BCAA transporter(s) (POI2) under the control of a low oxygen FNR promoter in vitro in a culture vessel (e.g., flask, fermenter or other vessel, e.g., used during with cell growth, cell expansion, fermentation, recovery, purification, formulation, and/or manufacture). In some embodiments, it is desirable to pre-load a strain with active BCAA catabolism enzyme(s) and/or BCAA transporter(s) prior to administration. This can be done by pre-inducing the expression of these enzymes as the strains are propagated, (e.g., in flasks, fermenters or other appropriate vesicles) and are prepared for in vivo administration. In some embodiments, strains are induced under anaerobic and/or low oxygen conditions, e.g. to induce FNR promoter activity and drive expression of one or more proteins of interest. In some embodiments, it is desirable to prepare, pre-load and pre-induce the strains under aerobic or microaerobic conditions with one or more proteins of interest. This allows more efficient growth and, in some cases, reduces the build-up of toxic metabolites.

FNRS24Y is a mutated form of FNR which is more resistant to inactivation by oxygen, and therefore can activate FNR promoters under aerobic conditions (see e.g., Jervis A J, The O2 sensitivity of the transcription factor FNR is controlled by Ser24 modulating the kinetics of [4Fe-4S] to [2Fe-2S] conversion, Proc Natl Acad Sci USA. 2009 Mar. 24; 106(12):4659-64, the contents of which is herein incorporated by reference in its entirety). The O2 sensitivity of the transcription factor FNR is controlled by Ser24 modulating the kinetics of [4Fe-4S] to [2Fe-2S] conversion, Proc Natl Acad Sci USA. 2009 Mar. 24; 106(12):4659-64, the contents of which is herein incorporated by reference in its entirety). In this oxygen bypass system, FNRS24Y is induced by addition of arabinose and then drives the expression of one or more POIs by binding and activating the FNR promoter under aerobic conditions. Thus, strains can be grown, produced or manufactured efficiently under aerobic conditions, while being effectively pre-induced and pre-loaded, as the system takes advantage of the strong FNR promoter resulting in of high levels of expression of one or more POIs. This system does not interfere with or compromise in vivo activation, since the mutated FNRS24Y is no longer expressed in the absence of arabinose, and wild type FNR then binds to the FNR promoter and drives expression of the POIs in vivo.

In some embodiments, a Lad promoter and IPTG induction are used in this system (in lieu of Para and arabinose induction). In some embodiments, a rhamnose inducible promoter is used in this system. In some embodiments, a temperature sensitive promoter is used to drive expression of FNRS24Y. Other inducible promoters may be used in this system, as are known in the art.

FIG. 79B depicts a strategy to allow the expression of one or more POI(s) under aerobic conditions through the arabinose inducible expression of FNRS24Y. By using a ribosome binding site optimization strategy, the levels of Fnr^{S24Y} expression can be fine-tuned, e.g., under optimal inducing conditions (adequate amounts of arabinose for full induction). Fine-tuning is accomplished by selection of an appropriate RBS with the appropriate translation initiation rate. Bioinformatics tools for optimization of RBS are known in the art.

FIG. 79C depicts a strategy to fine-tune the expression of a Para-POI construct by using a ribosome binding site optimization strategy. Bioinformatics tools for optimization of RBS are known in the art. In one strategy, arabinose controlled POI genes can be integrated into the chromosome to provide for efficient aerobic growth and pre-induction of the strain (e.g., in flasks, fermenters or other appropriate vesicles), while integrated versions of P_{fnrS}-POI constructs are maintained to allow for strong in vivo induction.

FIG. 80A depicts a schematic of the gene organization of a PssB promoter. The ssB gene product protects ssDNA from degradation; SSB interacts directly with numerous enzymes of DNA metabolism and is believed to have a central role in organizing the nucleoprotein complexes and processes involved in DNA replication (and replication restart), recombination and repair. The PssB promoter was cloned in front of a LacZ reporter and beta-galactosidase activity was measured. FIG. 80B depicts a bar graph showing the reporter gene activity for the PssB promoter under aerobic and anaerobic conditions. Briefly, cells were grown aerobically overnight, then diluted 1:100 and split into two different tubes. One tube was placed in the anaerobic chamber, and the other was kept in aerobic conditions for the length of the experiment. At specific times, the cells were analyzed for promoter induction. The Pssb promoter is active under aerobic conditions, and shuts off under anaerobic conditions. This promoter can be used to express a gene of interest under aerobic conditions. This promoter can also be used to tightly control the expression of a gene product such that it is only expressed under anaerobic and/or low oxygen conditions. In this case, the oxygen induced PssB promoter induces the expression of a repressor, which represses the expression of a gene of interest. Thus, the gene of interest is only expressed in the absence of the repressor, i.e., under anaerobic and/or low oxygen conditions. This strategy has the advantage of an additional level of control for improved fine-tuning and tighter control. In one non-limiting example, this strategy can be used to control expression of thyA and/or dapA, e.g., to make a conditional auxotroph. The chromosomal copy of dapA or ThyA is knocked out. Under anaerobic and/or low oxygen conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can, for example be employed to allow survival of bacteria under anaerobic and/or low oxygen conditions, e.g., the gut, but prevent survival under aerobic conditions (biosafety switch).

FIG. 81 depicts β-galactosidase levels in samples comprising bacteria harboring a low-copy plasmid expressing lacZ from an FNR-responsive promoter selected from the exemplary FNR promoters. Different FNR-responsive promoters were used to create a library of anaerobic-inducible reporters with a variety of expression levels and dynamic ranges. These promoters included strong ribosome binding sites. Bacterial cultures were grown in either aerobic (+O2) or anaerobic conditions (−O2). Samples were removed at 4 hrs and the promoter activity based on β-galactosidase levels was analyzed by performing standard β-galactosidase colorimetric assays.

FIG. 82A depicts a schematic representation of the lacZ gene under the control of an exemplary FNR promoter (P_{fnrS}). LacZ encodes the β-galactosidase enzyme and is a common reporter gene in bacteria. FIG. 82B depicts FNR promoter activity as a function of β-galactosidase activity in SYN340. SYN340, an engineered bacterial strain harboring a low-copy fnrS-lacZ fusion gene, was grown in the presence or absence of oxygen. Values for standard β-galactosidase colorimetric assays are expressed in Miller units (Miller, 1972). These data suggest that the fnrS promoter begins to drive high-level gene expression within 1 hr under anaerobic conditions. FIG. 82C depicts the growth of bacterial cell cultures expressing lacZ over time, both in the presence and absence of oxygen.

FIGS. 83A and 83B depict ATC (FIG. 83A) or nitric oxide-inducible (FIG. 83B) reporter constructs. These constructs, when induced by their cognate inducer, lead to expression of GFP. Nissle cells harboring plasmids with either the control, ATC-inducible Pₜₑₜ-GFP reporter construct or the nitric oxide inducible P_{nsrR}-GFP reporter construct induced across a range of concentrations. Promoter activity is expressed as relative florescence units. FIG. 83C depicts a schematic of the constructs. FIG. 83D depicts a dot blot of bacteria harboring a plasmid expressing NsrR under control of a constitutive promoter and the reporter gene gfp (green fluorescent protein) under control of an NsrR-inducible promoter. DSS-treated mice serve as exemplary models for HE. As in HE subjects, the guts of mice are damaged by supplementing drinking water with 2-3% dextran sodium sulfate (DSS). Chemiluminescent is shown for NsrR-regulated promoters induced in DSS-treated mice.

FIG. 84 depicts the prpR propionate-responsive inducible promoter. The sequence for one propionate-responsive promoter is also disclosed herein as SEQ ID NO: 13.

FIGS. 85A and 85B depict a schematic diagram of a wild-type clbA construct (upper panel) and a schematic diagram of a clbA knockout construct (lower panel).

FIG. 86 depicts exemplary sequences of a wild-type clbA construct (SEQ ID NO: 141) and a clbA knock-out construct (SEQ ID NO: 142).

FIG. 87 depicts a schematic of non-limiting processes for designing and producing the genetically engineered bacteria of the present disclosure. The step of “defining” comprises 1. Identification of diverse candidate approaches based on microbial physiology and disease biology; 2. Use of bioinformatics to determine candidate metabolic pathways; the use of prospective tools to determine performance targets required of optimized engineered synthetic biotics. The step of “designing” comprises the use of 1. Cutting-edge DNA assembly to enable combinatorial testing of pathway organization; 2. Mathematical models to predict pathway efficiency; 3. Internal stable of proprietary switches and parts to permit control and tuning of engineered circuits. The step of “Building” comprises 1. Building core structures “chassies” 2. Stably integrating engineered circuits into optimal chromosomal locations for efficient expression; 3. Employing unique functional assays to assess genetic circuit fidelity and activity. The step of “integrating” comprises 1. Use of chromosomal markers, which enable monitoring of synthetic biotic localization and transit times in animal models; 2. Leveraging expert microbiome network and bioinformatics support to expand understanding of how specific disease states affect GI microbial flora and the behaviors of synthetic biotics in that environment; 3. Activating process development research and optimization in-house during the discovery phase, enabling rapid and seamless transition of development candidates to pre-clinical progression; Drawing upon extensive experience in specialized disease animal model refinement, which supports prudent, high quality testing of candidate synthetic biotics.

FIG. 88 depicts a schematic of non-limiting manufacturing processes for upstream and downstream production of the genetically engineered bacteria of the present disclosure. Step A depicts the parameters for starter culture 1 (SC1): loop full—glycerol stock, duration overnight, temperature 37° C., shaking at 250 rpm. Step B depicts the parameters for starter culture 2 (SC2): 1/100 dilution from SC1, duration 1.5 hours, temperature 37° C., shaking at 250 rpm. Step C depicts the parameters for the production bioreactor: inoculum—SC2, temperature 37° C., pH set point 7.00, pH dead band 0.05, dissolved oxygen set point 50%, dissolved oxygen cascade agitation/gas FLO, agitation limits 300-1200 rpm, gas FLO limits 0.5-20 standard liters per minute, duration 24 hours. Step D depicts the parameters for harvest: centrifugation at speed 4000 rpm and duration 30 minutes, wash 1×10% glycerol/PBS, centrifugation, re-suspension 10% glycerol/PBS. Step E depicts the parameters for vial fill/storage: 1-2 mL aliquots, −80° C.

### DETAILED DESCRIPTION

The disclosure includes engineered and programmed microorganisms, e.g., bacteria, yeast, and viruses, pharmaceutical compositions thereof, and methods of modulating and treating disorders involving the catabolism of a branched chain amino acid, such as leucine, valine, and isoleucine. In some embodiments, the microorganism, e.g., bacterium, yeast, or virus, has been engineered to comprise heterologous gene sequence(s) encoding one or more branched chain amino acid catabolism enzyme(s). In some embodiments, the engineered microorganism, e.g., engineered bacterium, comprises heterologous gene sequence(s) encoding one or more branched chain amino acid catabolism enzyme(s) and is capable of reducing the level of one or more branched chain amino acids and/or corresponding alpha-keto acid(s) and/or other corresponding metabolite(s). For example, the engineered bacterium, may comprise a BCAA transporter, such as livKHMGF and/or BrnQ. In some embodiments, the engineered bacterium comprises heterologous gene sequence(s) encoding one or more branched chain amino acid catabolism enzyme(s) and is capable of metabolizing one or more branched chain amino acids and/or corresponding alpha-keto acid(s) and/or other corresponding metabolite(s). In some embodiments, the engineered bacterium comprises heterologous gene sequence(s) encoding one or more branched chain amino acid catabolism enzyme(s) and is capable of transporting one or more branched chain amino acids and/or corresponding alpha-keto acid(s) and/or other corresponding metabolite(s) into the bacterium. In some embodiments, the engineered bacterium comprises heterologous gene sequence(s) encoding one or more branched chain amino acid catabolism enzyme(s) and is capable of reducing the level of and/or metabolizing one or more branched chain amino acids and/or corresponding alpha-keto acid(s) and/or other corresponding metabolite(s) in low-oxygen environments, e.g., the gut. In some embodiments, the engineered bacteria convert the branched chain amino acid(s) and/or corresponding alpha-keto acid(s) and/or other corresponding metabolite(s) to a non-toxic or low toxicity metabolite, e.g., isovaleraldehyde, isobutyraldehyde, 2-methylbutyraldehyde, isovaleric acid, isobutyric acid, 2-methylbutyric acid, isopentanol, isobutanol, and 2-methylbutanol. In some embodiments, the engineered bacterium comprises a genetic modification that reduces export of a branched chain amino acid from the bacterial cell, for example, the bacterial cell may comprise a knockout or knock-down of a gene that encodes a BCAA exporter, such as leuE (which encodes a leucine exporter). In some embodiments, the engineered bacterium comprises gene sequence(s) or gene cassette(s) encoding one or more transporters of a branched chain amino acid, which transporters function to import one or more BCAA(s) into the bacterial cell. In some embodiments, the bacterium has been engineered to comprise a genetic modification that reduces or inhibits endogenous production of one or more branched chain amino acids and/or one or more corresponding alpha-keto acids or other metabolite(s), for example, the bacterium may comprise a knockout or knock-down of a gene that encodes a molecule required for BCAA synthesis, such as IlvC (which encodes keto acid reductoisomerase). In some embodiments, the bacterium has been engineered to comprise an auxotroph, including, for example, a BCAA auxotrophy, such as IlvC (which is required for BCAA synthesis and requires the cell to import isoleucine and valine to survive) or other auxotrophy, as provided herein and known in the art, e.g., thyA auxotrophy. In some embodiments, the bacterium has been engineered to comprise a kill-switch, such as any of the kill-switches provided herein and known in the art. In some embodiments, the bacterium has been engineered to comprise antibiotic resistance, such as any of the antibiotic resistance provided herein and known in the art. In any of these embodiments, the gene sequence(s) encoding one or more branched chain amino acid catabolism enzyme(s), transporter(s), and other molecules can be integrated into the bacterial chromosome and/or can be present on a plasmid(s) (low copy and/or high copy). In any of these embodiments, the gene sequence(s) encoding one or more branched chain amino acid catabolism enzyme(s), transporter(s), and other molecules can be under the control of an inducible or constitutive promoter. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline.

Thus, the recombinant bacterial cells and pharmaceutical compositions comprising the bacterial cells disclosed herein may be used to catabolize branched chain amino acids, e.g., leucine, isoleucine, valine, and/or their corresponding alpha-keto acid counterparts, to modify, ameliorate, treat and/or prevent conditions associated with disorders involving the catabolism of a branched chain amino acid. In one embodiment, the disorder involving the catabolism of a branched chain amino acid is a metabolic disorder involving the abnormal catabolism of a branched chain amino acid, including but not limited to maple syrup urine disease (MSUD), isovaleric acidemia, propionic acidemia, methylmalonic acidemia, or diabetes ketoacidosis, 3-MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria. In another embodiment, the disorder involving the catabolism of a branched chain amino acid is a disorder caused by the activation of mTor, for example, cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “recombinant microorganism” refers to a microorganism, e.g., bacterial, yeast or viral cell, or bacteria, yeast or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial, yeast, or viral cell, or bacteria, yeast, or virus, that has been genetically modified from its native state to perform a specific function. Thus, a “programmed or engineered bacterial cell” or “programmed or engineered bacteria” refers to a bacterial cell or bacteria that has been genetically modified from its native state to perform a specific function, e.g., to metabolize a branched chain amino acid. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell and can be a native sequence (naturally found or expressed in the cell) or non-native sequence (not naturally found or expressed in the cell) and can be a natural or wild-type sequence or a variant, non-natural, or synthetic sequence. “Heterologous gene” includes a native gene, or fragment thereof, that has been introduced into the host cell in a form that is different from the corresponding native gene. For example, a heterologous gene may include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria, yeast, or virus, or a sequence that is modified and/or mutated as compared to the unmodified sequence from bacteria, yeast, or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered microorganism of the disclosure comprises a gene that is operably linked to a promoter that is not associated with said gene in nature. For example, in some embodiments, the genetically engineered bacteria disclosed herein comprise a gene that is operably linked to a directly or indirectly inducible promoter that is not associated with said gene in nature, e.g., an FNR responsive promoter (or other promoter disclosed herein) operably linked to a gene encoding a branched chain amino acid catabolism enzyme. In some embodiments, the genetically engineered yeast or virus of the disclosure comprises a gene that is operably linked to a directly or indirectly inducible promoter that is not associated with said gene in nature, e.g., a promoter operably linked to a gene encoding a branched chain amino acid catabolism enzyme.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, signal sequences, and stem-loop structures. In one embodiment, the regulatory sequence comprises a promoter, e.g., an FNR responsive promoter or another promoter disclosed herein.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding a branched chain amino acid catabolism enzyme, that is joined to a regulatory sequence in a manner which allows expression of the nucleic acid sequence, e.g., the gene encoding the branched chain amino acid catabolism enzyme. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive. A “constitutive promoter” refers to a promoter that allows for continual transcription of the coding sequence or gene under its control.

“Constitutive promoter” refers to a promoter that is capable of facilitating continuous transcription of a coding sequence or gene under its control and/or to which it is operably linked. Constitutive promoters and variants are well known in the art and include, but are not limited to, Ptac promoter, BBa_J23100, a constitutive Escherichia coli σ^{S} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{azaC} promoter, a P_{araBAD} promoter, and a P_{TetR} promoter, each of which are described in more detail herein. Examples of other inducible promoters are provided herein below.

As used herein, “stably maintained” or “stable” bacterium is used to refer to a bacterial host cell carrying non-native genetic material, e.g., a gene encoding a branched chain amino acid catabolism enzyme, which is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising a gene encoding a branched chain amino acid catabolism enzyme, in which the plasmid or chromosome carrying the gene is stably maintained in the bacterium, such that branched chain amino acid catabolism enzyme can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide.

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene encoding a branched chain amino acid catabolism enzyme.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, base substitution, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising a branched chain amino acid catabolism enzyme operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

Specifically, the term “genetic modification that reduces export of a branched chain amino acid from the bacterial cell” refers to a genetic modification that reduces the rate of export or quantity of export of a branched chain amino acid from the bacterial cell, as compared to the rate of export or quantity of export of the branched chain amino acid from a bacterial cell not having said modification, e.g., a wild-type bacterial cell. In one embodiment, a recombinant bacterial cell having a genetic modification that reduces export of a branched chain amino acid from the bacterial cell comprises a genetic mutation in a native gene, e.g., a leuE gene. In another embodiment, a recombinant bacterial cell having a genetic modification that reduces export of a branched chain amino acid from the bacterial cell comprises a genetic mutation in a native promoter, e.g., a leuE promoter, which reduces or inhibits transcription of the leuE gene. In another embodiment, a recombinant bacterial cell having a genetic modification that reduces export of a branched chain amino acid from the bacterial cell comprises a genetic mutation leading to overexpression of a repressor of an exporter of a branched chain amino acid. In another embodiment, a recombinant bacterial cell having a genetic modification that reduces export of a branched chain amino acid from the bacterial cell comprises a genetic mutation which reduces or inhibits translation of the gene encoding the exporter, e.g., the leuE gene.

Moreover, the term “genetic modification that increases import of a branched chain amino acid into the bacterial cell” refers to a genetic modification that increases the uptake rate or increases the uptake quantity of a branched chain amino acid into the cytosol of the bacterial cell, as compared to the uptake rate or uptake quantity of the branched chain amino acid into the cytosol of a bacterial cell not having said modification, e.g., a wild-type bacterial cell. In some embodiments, an engineered bacterial cell having a genetic modification that increases import of a branched chain amino acid into the bacterial cell refers to a bacterial cell comprising heterologous gene sequence (native or non-native) encoding one or more importer(s) (transporter(s)) of a branched chain amino acid. In some embodiments, the genetically engineered bacteria comprising genetic modification that increases import of a branched chain amino acid into the bacterial cell comprise gene sequence(s) encoding a BCAA transporter or other amino acid transporter that transports one or more BCAA(s) into the bacterial cell, for example a transporter that is capable of transporting leucine, valine, and/or isoleucine into a bacterial cell. The transporter can be any transporter that assists or allows import of a BCAA into the cell. In certain embodiments, the BCAA transporter is a leucine transporter, e.g., a high-affinity leucine transporter, e.g., LivKHMGF. In certain embodiments, the engineered bacterial cell contains gene sequence encoding one or more livK, livH, livM, livG, and livF genes. In certain embodiments, the BCAA transporter is a BCAA transporter, e.g., a low affinity BCAA transporter, e.g., BrnQ. In certain embodiments, the engineered bacterial cell contains gene sequence encoding brnQ gene. In some embodiments, the engineered bacteria comprise more than one copy of gene sequence encoding a BCAA transporter. In some embodiments, the engineered bacteria comprise gene sequence(s) encoding more than one BCAA transporter, e.g., two or more different BCAA transporters.

As used herein, the term “transporter” is meant to refer to a mechanism, e.g., protein, proteins, or protein complex, for importing a molecule, e.g., amino acid, peptide (di-peptide, tri-peptide, polypeptide, etc.), toxin, metabolite, substrate, as well as other biomolecules into the microorganism from the extracellular milieu.

As used herein, the term “polypeptide of interest” or “polypeptides of interest”, “protein of interest”, “proteins of interest”, “payload”, “payloads” includes, but is not limited to, any or a plurality of any of the branched chain amino acid catabolism enzymes, and/or branched chain amino acid transporters, and/or branched chain amino acid binding proteins and/or branched chain amino acid exporters described herein. As used herein, the term “gene of interest” or “gene sequence of interest” includes any or a plurality of any of the gene(s) an/or gene sequence(s) and or gene cassette(s) encoding one or more branched chain amino acid catabolism enzymes, ranched chain amino acid transporters, branched chain amino acid binding proteins, and branched chain amino acid exporters described herein.

The term “branched chain amino acid” or “BCAA,” as used herein, refers to an amino acid which comprises a branched side chain. Leucine, isoleucine, and valine are naturally occurring amino acids comprising a branched side chain. However, non-naturally occurring, usual, and/or modified amino acids comprising a branched side chain are also encompassed by the term branched chain amino acid.

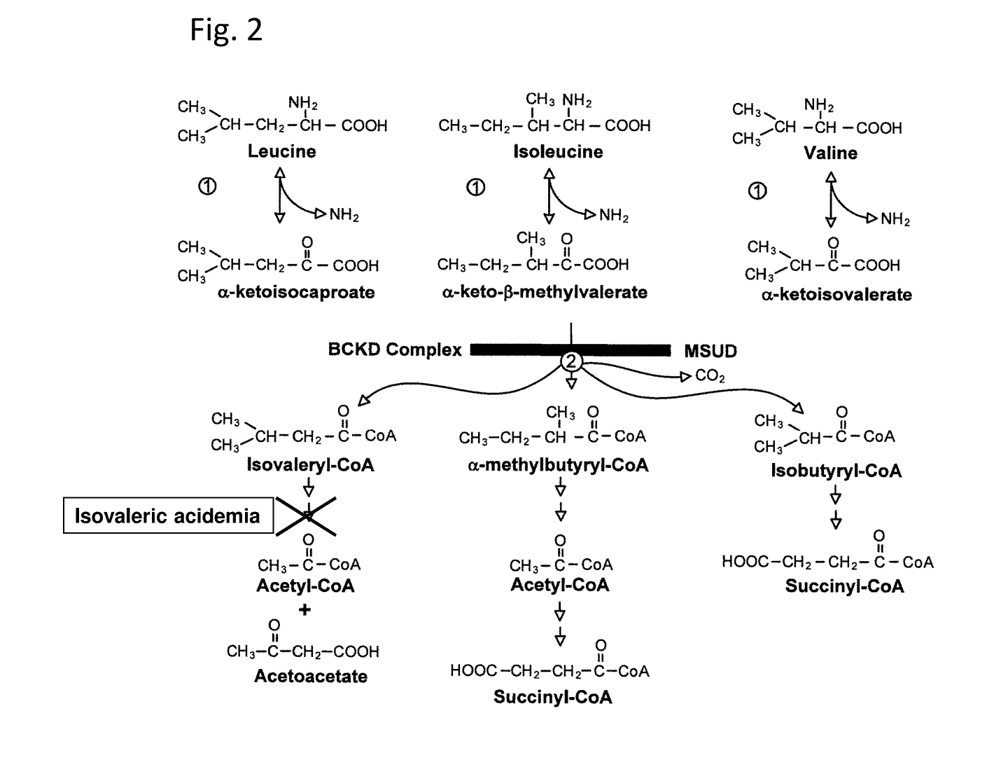

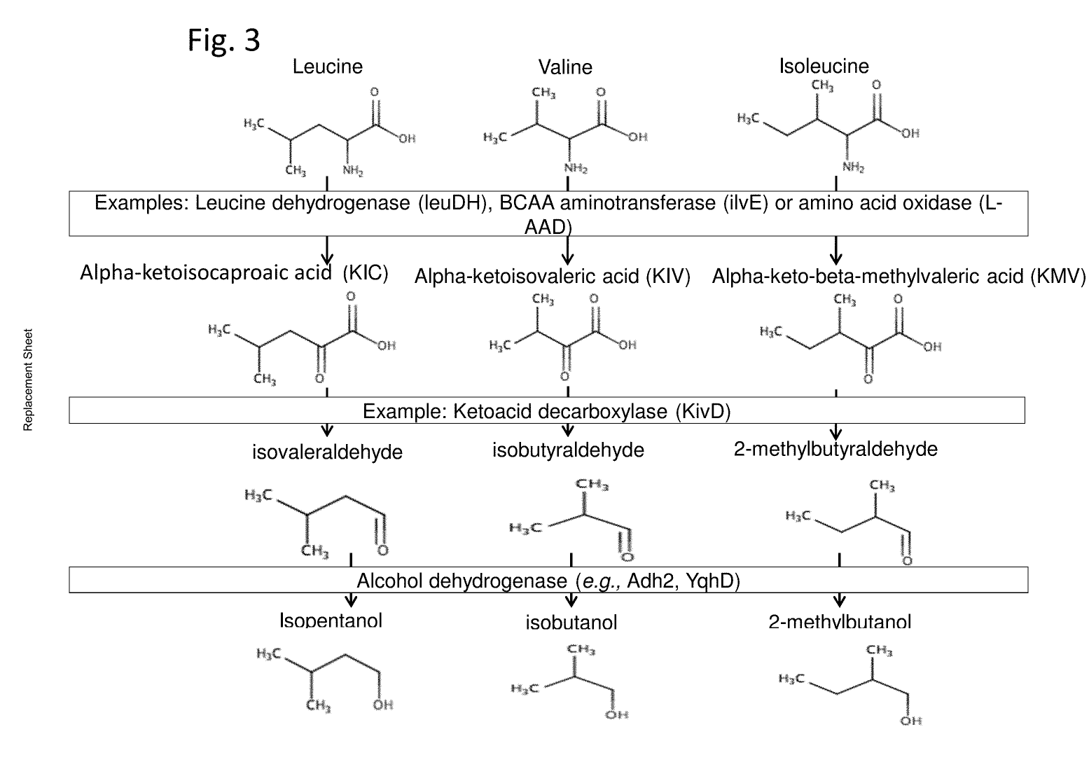

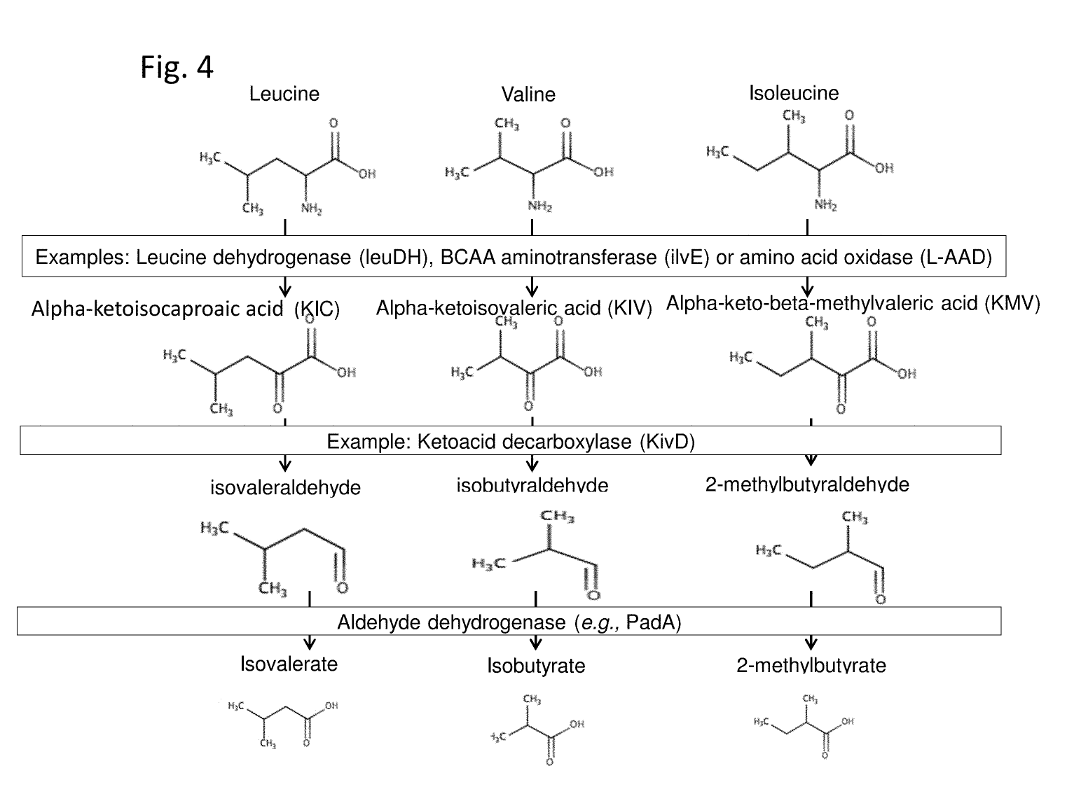

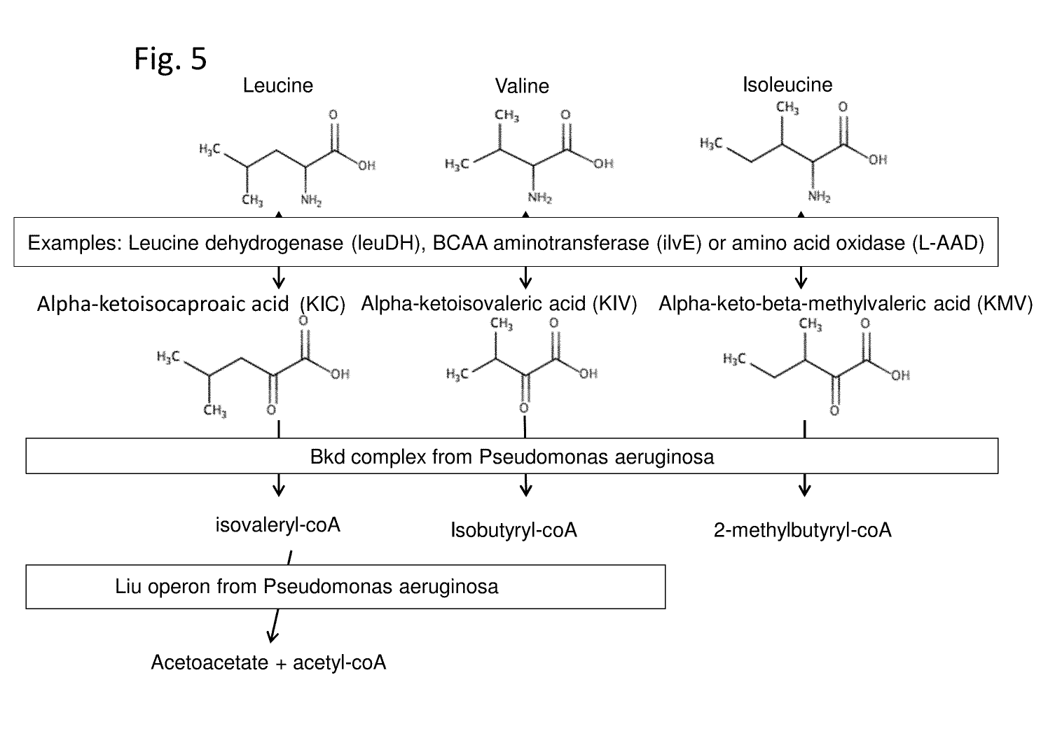

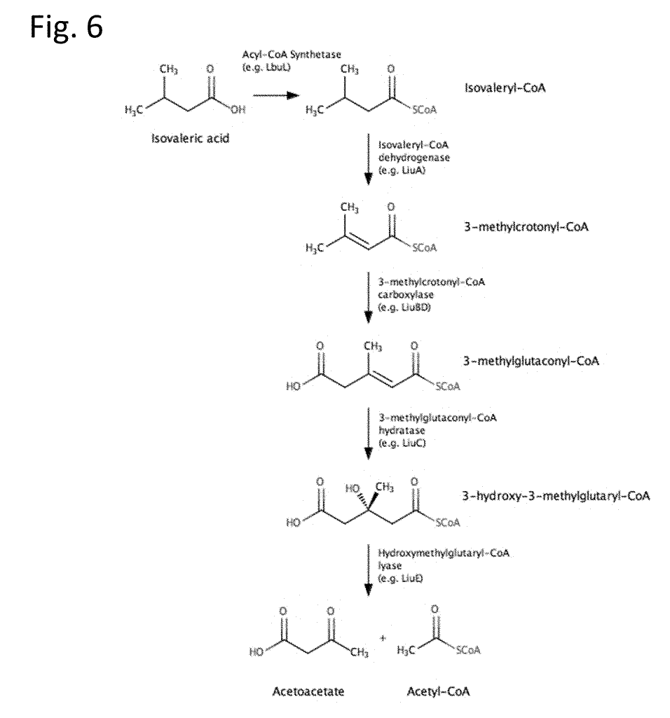

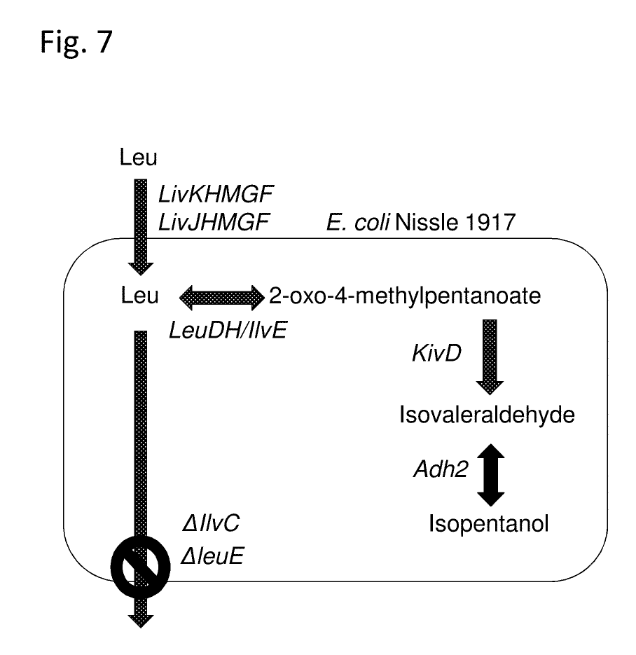

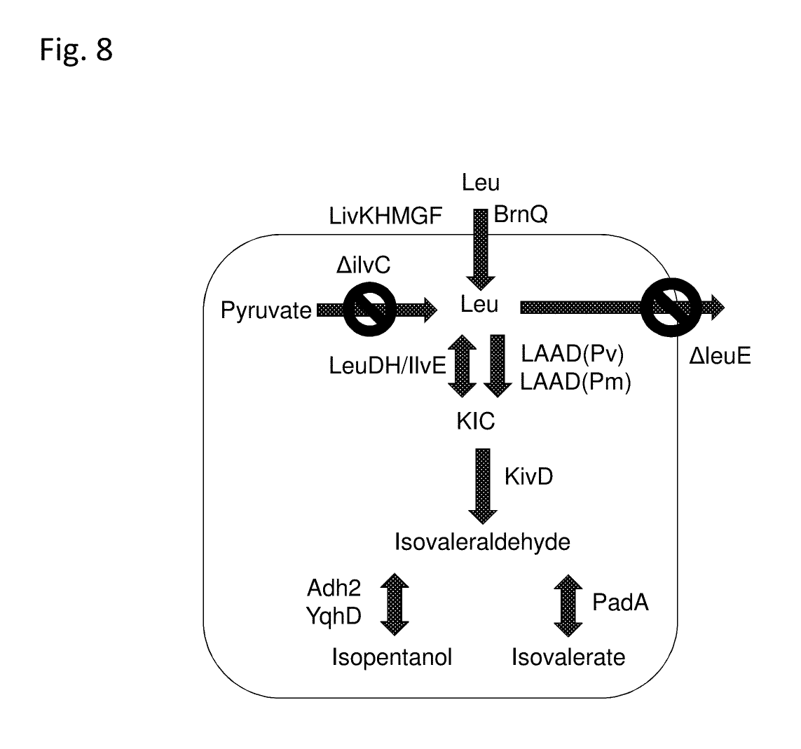

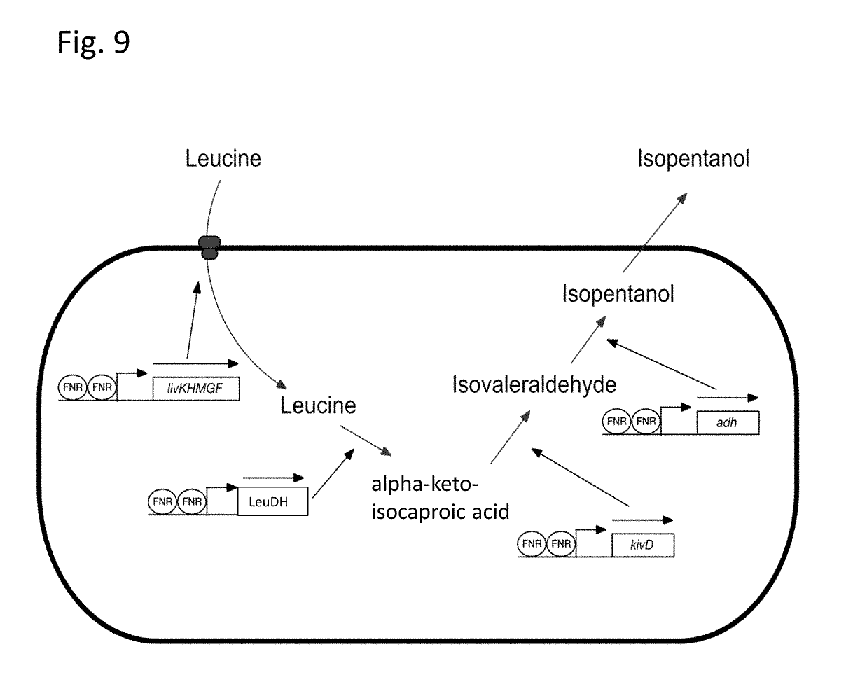

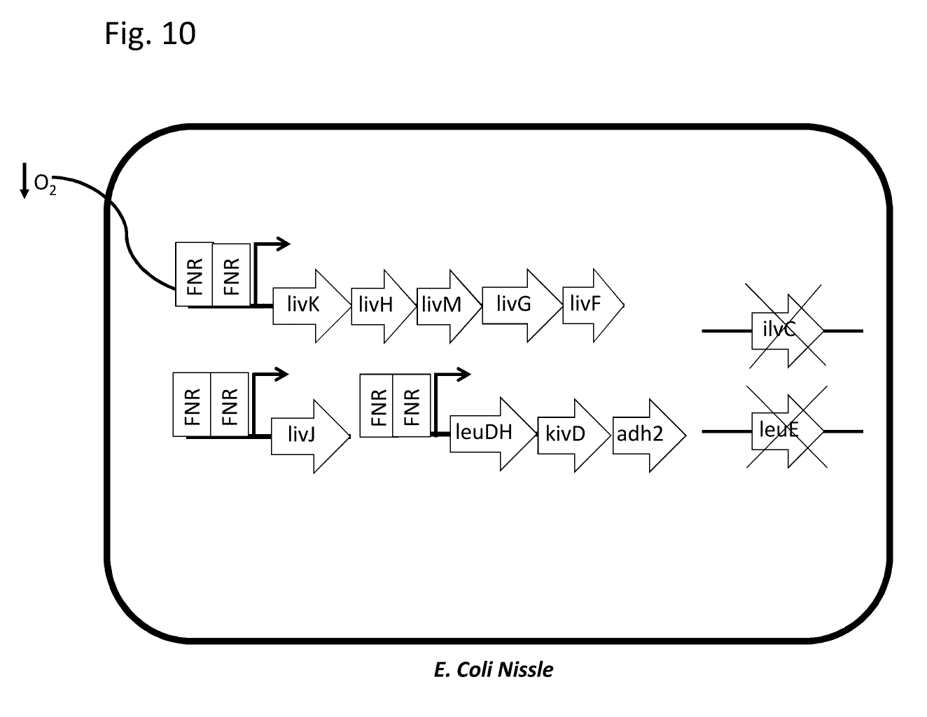

Conversion of a branched chain amino acid to its corresponding alpha-keto acid is the first step in branched chain amino acid catabolism and is reversible when catalyzed by a leucine dehydrogenase (leuDH) or a branched chain amino acid aminotransferase (ilvE), or irreversible when catalyzed by an amino acid oxidase (also referred as amino acid deaminase, e.g., L-AAD). As used herein, the terms “alpha-keto acid” or “α-keto acid” refers to the molecules which are produced after deamination of a branched chain amino acid, and include the naturally occurring alpha-keto acids: α-ketoisocaproic acid (KIC) (also known as 4-methyl-2-oxopentanoate), α-ketoisovaleric acid (KIV) (also known as 2-oxoisopentanoate), and α-keto-beta-methylvaleric acid (KMV) (also known as 3-methyl-2-oxopentanoate). However, non-naturally occurring, unusual, or modified alpha-keto acids are also encompassed by the term “alpha-keto acid.” See FIGS. 2-4.

Conversion of a branched chain alpha-keto acid to its corresponding branched acyl-CoA derivative is the second step in branched chain amino acid catabolism and is irreversible. As used herein, the term “acyl-CoA derivative” refers to the molecules which are produced after dehydrogenation of a branched chain alpha-keto acid, and include the naturally occurring acyl-CoA derivatives, propionyl-CoA and acetyl-CoA (See FIG. 2) However, non-naturally occurring, unusual, or modified acyl-CoA derivatives are also encompassed by the term “acyl-CoA derivative.”

In an alternative BCAA catabolism pathway, known as the “Ehrlich pathway”, a branched chain alpha-keto acid is irreversibly decarboxylated to its corresponding branched chain amino acid-derived aldehyde. This irreversible catabolic conversion of a branched chain alpha-keto acid, e.g., alpha-keto acids α-ketoisocaproic acid (KIC), α-ketoisovaleric acid (KIV), or α-keto-beta-methylvaleric acid (KMV) to its corresponding branched chain amino acid-derived aldehyde, e.g., isovaleraldehyde, isobutyraldehyde, or 2-methylbutyraldehyde, is catalyzed by ketoacid decarboxylase (KivD). As used herein, the term “branched chain amino acid-derived aldehyde” refers to the molecules which are produced after decarboxylation of a branched chain alpha-keto acid, and include the naturally occurring aldehydes isovaleraldehyde, 2-methyl butyraldehyde, and isobutyraldehyde. However, non-naturally occurring, unusual, or modified aldehydes are also encompassed by the term “aldehyde.” BCAA-derived aldehydes can then be converted to alcohols (e.g., isopentanol, isobutanol, 2-methylbutanol) by an alcohol dehydrogenase, e.g., Adh2 or Ygh.D. Alternatively, BCAA-derived aldehydes can be converted to their respective carboxylic acids (e.g., isovalerate, isobutyrate, and 2-methylbutyrate) by an aldehyde dehydrogenase, e.g., PadA.

As used herein, the phrase “exogenous environmental condition” or “exogenous environment signal” refers to settings, circumstances, stimuli, or biological molecules under which a promoter described herein is directly or indirectly induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). In some embodiments, the exogenous environmental condition is specific to a branched chain amino acid catabolic enzyme disease, e.g., MSUD. In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrS, FNR, P-FNRS promoter and other such related designations to indicate the promoter PfnrS.

TABLE 1
Examples of transcription factors and
responsive genes and regulatory regions

| Transcription | Examples of responsive genes, |
|---|---|
| Factor | promoters, and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

In some embodiments, the exogenous environmental conditions are the presence or absence of reactive oxygen species (ROS). In other embodiments, the exogenous environmental conditions are the presence or absence of reactive nitrogen species (RNS). In some embodiments, exogenous environmental conditions are biological molecules that are involved in the inflammatory response, for example, molecules present in an inflammatory disorder of the gut. In some embodiments, the exogenous environmental conditions or signals exist naturally or are naturally absent in the environment in which the recombinant bacterial cell resides. In some embodiments, the exogenous environmental conditions or signals are artificially created, for example, by the creation or removal of biological conditions and/or the administration or removal of biological molecules.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut). “Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

As used herein, the term “low oxygen” is meant to refer to a level, amount, or concentration of oxygen (O2) that is lower than the level, amount, or concentration of oxygen that is present in the atmosphere (e.g., <21% O2; <160 torr O2)). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O2) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O2 that is 0-60 mmHg O2 (0-60 torr O2) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O2), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg O2, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorportated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table A summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stiffing) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE A

| Compartment | Oxygen Tension |
|---|---|
| stomach | ~60 torr (e.g., 58 +/− 15 torr) |
| duodenum and first | ~30 torr (e.g., 32 +/− 8 torr); |
| part of jejunum | ~20% oxygen in ambient air |
| Ileum (mid- small | ~10 torr; ~6% oxygen in ambient air |
| intestine) | (e.g., 11 +/− 3 torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, viruses, parasites, fungi, certain algae, yeast, and protozoa. In some aspects, the microorganism is engineered (“engineered microorganism”) to produce one or more therapeutic molecules, e.g., lysosomal enzyme(s). In certain embodiments, the engineered microorganism is an engineered bacterium. In certain embodiments, the engineered microorganism is an engineered yeast or virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis and Saccharomyces boulardii (Sonnenborn et al., 2009; Dinleyici et al., 2014; U.S. Pat. No. 6,835,376; U.S. Pat. No. 6,203,797; U.S. Pat. No. 5,589,168; U.S. Pat. No. 7,731,976). Non-pathogenic bacteria also include commensal bacteria, which are present in the indigenous microbiota of the gut. In one embodiment, the disclosure further includes non-pathogenic Saccharomyces, such as Saccharomyces boulardii. Naturally pathogenic bacteria may be genetically engineered to reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to, certain strains belonging to the genus Bifidobacteria, Escherichia Coli, Lactobacillus, and Saccharomyces e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum, and Saccharomyces boulardii (Dinleyici et al., 2014; U.S. Pat. No. 5,589,168; U.S. Pat. No. 6,203,797; U.S. Pat. No. 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “auxotroph” or “auxotrophic” refers to an organism that requires a specific factor, e.g., an amino acid, a sugar, or other nutrient) to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Diseases associated with the catabolism of a branched chain amino acid, e.g., MSUD, may be caused by inborn genetic mutations for which there are no known cures. Diseases can also be secondary to other conditions, e.g., liver diseases. Treating diseases involving the catabolism of a branched chain amino acid, such as MSUD, may encompass reducing normal levels of branched chain amino acids, reducing excess levels of branched chain amino acids, or eliminating branched chain amino acids, e.g., leucine, and does not necessarily encompass the elimination of the underlying disease.

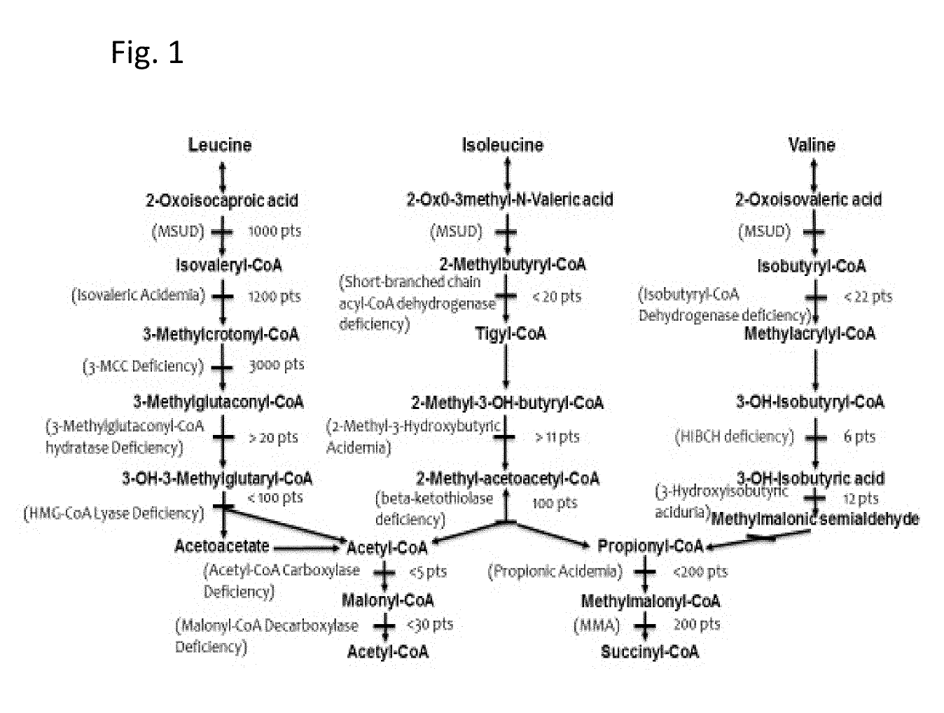

As used herein, the term “catabolism” refers to the breakdown of a molecule into a smaller unit. As used herein, the term “branched chain amino acid catabolism” refers to the conversion of a branched chain amino acid, such as leucine, isoleucine, or valine, into a corresponding metabolite, for example a corresponding alpha keto acid, acyl-CoA derivative, aldehyde, alcohol, or other metabolite, such as any of the BCAA metabolites disclosed herein; or the conversion of a branched chain alpha keto acid into its corresponding acyl-CoA derivative, aldehydes, alcohols or other metabolites, such as any of the BCAA metabolites disclosed herein. The “branched chain amino acid catabolism” refers to both native and non-native conversion of a branched chain amino acid, such as leucine, isoleucine, or valine, into a corresponding metabolite. Thus, the term additionally covers catabolism of BCAA that may not occur in nature and is artificially induced as a result of genetic engineering. In one embodiment, “abnormal catabolism” refers to a decrease in the rate or the level of conversion of a branched chain amino acid or its corresponding alpha-keto acid to a corresponding metabolite, leading to the accumulation of the branched chain amino acid, accumulation of the branched chain alpha-keto acid, and/or accumulation of a BCAA metabolite that is toxic or that accumulates to a toxic level in a subject (e.g., see FIG. 1). In one embodiment, the branched chain amino acid, branched chain alpha-keto acid, or other metabolite thereof, accumulates to a toxic level in the blood or the brain of a subject, leading to the development of a disease or disorder associated with the abnormal catabolism of the branched chain amino acid in the subject. In one embodiment, “abnormal leucine catabolism” refers to a level of greater than 4 mg/dL of leucine in the plasma of a subject. In another embodiment, “normal leucine catabolism” refers to a level of less than 4 mg/dL of leucine in the plasma of a subject.

As used herein, the term “disorder involving the abnormal catabolism of a branched amino acid” or “disease involving the abnormal catabolism of a branched amino acid” or “branched chain amino acid disease” or “disease associated with excess branched chain amino acid” refers to a disease or disorder wherein the catabolism of a branched chain amino acid or a branched chain alpha-keto acid is abnormal. Such diseases are genetic disorders that result from deficiency in at least one of the enzymes required to catabolize a branched chain amino acid, e.g., leucine, isoleucine, or valine. As a result, individuals suffering from branched chain amino acid disease have accumulated branched chain amino acids in their cells and tissues. Examples of branched chain amino acid diseases include, but are not limited to, MSUD, isovaleric acidemia, 3-MCC deficiency, 3-methylglutaconyl-CoA hydrolase deficiency, HMG-CoA lysate deficiency, Acetyl CoA carboxylase deficiency, malonylCoA decarboxylase deficiency, short branched chain acyl-CoA dehydrogenase, 2-methyl-3-hydroxybutyric acidemia, beta-ketothiolase deficiency, isobutyl-CoA dehydrogenase deficiency, HIBCH deficiency, 3-hydroxyisobutyric aciduria, proprionic acidemis, methylmalonic acidemia, as well as those diseases resulting from mTor activation, including but not limited to cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia. In one embodiment, a “disease or disorder involving the catabolism of a branched chain amino acid” is a metabolic disease or disorder involving the abnormal catabolism of a branched chain amino acid. In another embodiment, a “disease or disorder involving the catabolism of a branched chain amino acid” is a disease or disorder caused by the activation of mTor. In one embodiment, the activation of mTor is abnormal.

In one embodiment, “abnormal catabolism” refers to a decrease in the rate or the level of conversion of the branched chain amino acid or its alpha-keto acid counterpart, leading to the accumulation of the branched chain amino acid or alpha-keto acid in a subject. In one embodiment, accumulation of the branched chain amino acid in the blood or the brain of a subject becomes toxic and leads to the development of a disease or disorder associated with the abnormal catabolism of the branched chain amino acid in the subject.

As used herein, the term “disease caused by the activation of mTor” or “disorder caused by the activation of mTor” refers to a disease or a disorder wherein the levels of branched chain amino acid may be normal, and wherein the branched chain amino acid causes the activation of mTor at a level higher than the normal level of mTor activity. In another embodiment, the subject having a disorder caused by the activation of mTor may have higher levels of a branched chain amino acid than normal. Diseases caused by the activation of mTor are known in the art. See, for example, Laplante and Sabatini, Cell, 149(2):74-293, 2012. As used herein, the term “disease caused by the activation of mTor” includes cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia. In one embodiment, the subject has normal levels of a branched chain amino acid, such as leucine, before administration of the engineered bacteria of the present disclosure. In another embodiment, the subject has decreased levels of the branched chain amino acid after the administration of the engineered bacteria of the present disclosure, thereby decreasing the levels of mTor or the activity of mTor, thereby treating the disorder in the subject. In one embodiment, the pharmaceutical composition disclosed herein decreases the activity of mTor by at least about 2-fold, 3-fold, 4-fold, or 5-fold in the subject.

As used herein, the term “anabolism” refers the conversion of a branched chain alpha-keto acid or an acyl-CoA derivative or other metabolite into its corresponding branched chain amino acid, such as leucine, isoleucine, or valine or alpha-keto acid, respectively.

As used herein a “pharmaceutical composition” refers to a preparation of genetically engineered microorganism of the disclosure, e.g., genetically engineered bacteria yeast or virus, with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, sodium bicarbonate calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., lysosomal storage disease (LSD). A therapeutically effective amount may, for example, be sufficient to treat, prevent, reduce the severity, delay the onset, and/or reduce the risk of occurrence of one or more symptoms of a disorder associated with lysosomal storage disease. A therapeutically effective amount, as well as a therapeutically effective frequency of administration, can be determined by methods known in the art and discussed below.

As used herein, the term “bacteriostatic” or “cytostatic” refers to a molecule or protein which is capable of arresting, retarding, or inhibiting the growth, division, multiplication or replication of recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “branched chain amino acid catabolic or catabolism enzyme” or “BCAA catabolic or catabolism enzyme” or “branched chain or BCAA amino acid metabolic enzyme” refers to any enzyme that is capable of metabolizing a branched chain amino acid or capable of reducing accumulated branched chain amino acid or that can lessen, ameliorate, or prevent one or more branched chain amino acid diseases or disease symptoms. Examples of branched chain amino acid metabolic enzymes include, but are not limited to, leucine dehydrogenase (e.g., LeuDH), branched chain amino acid aminotransferase (e.g., IlvE), branched chain α-ketoacid dehydrogenase (e.g., KivD), L-Amino acid deaminase (e.g., L-AAD), alcohol dehydrogenase (e.g., Adh2, YqhD)), and aldehyde dehydrogenase (e.g., PadA), and any other enzymes that catabolizes BCAA. Functional deficiencies in these proteins result in the accumulation of BCAA or its corresponding α-keto acid in cells and tissues. BCAA metabolic enzymes of the present disclosure include both wild-type or modified BCAA metabolic enzymes and can be produced using recombinant and synthetic methods or purified from nature sources. BCAA metabolic enzymes include full-length polypeptides and functional fragments thereof, as well as homologs and variants thereof. BCAA metabolic enzymes include polypeptides that have been modified from the wild-type sequence, including, for example, polypeptides having one or more amino acid deletions, insertions, and/or substitutions and may include, for example, fusion polypeptides and polypeptides having additional sequence, e.g., regulatory peptide sequence, linker peptide sequence, and other peptide sequence.

As used herein, “payload” refers to one or more molecules of interest to be produced by a genetically engineered microorganism, such as a bacterium, yeast, or a virus. In some embodiments, the payload is a therapeutic payload, e.g., a branched chain amino acid catabolic enzyme or a BCAA transporter polypeptide. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments, the payload comprises a repressor element, such as a kill switch. In some embodiments, the payload comprises an antibiotic resistance gene or genes. In some embodiments, the payload is encoded by a gene, multiple genes, gene cassette, or an operon. In alternate embodiments, the payload is produced by a biosynthetic or biochemical pathway, wherein the biosynthetic or biochemical pathway may optionally be endogenous to the microorganism. In alternate embodiments, the payload is produced by a biosynthetic or biochemical pathway, wherein the biosynthetic or biochemical pathway is not endogenous to the microorganism. In some embodiments, the genetically engineered microorganism comprises two or more payloads.

As used herein, the term “conventional branched chain amino acid or BCAA disease treatment” or “conventional branched chain amino acid or BCAA disease therapy” refers to treatment or therapy that is currently accepted, considered current standard of care, and/or used by most healthcare professionals for treating a disease or disorder associated with BCAA. It is different from alternative or complementary therapies, which are not as widely used.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria, yeast, or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting a biomolecule, e.g., polypeptide from the microbial, e.g., bacterial cytoplasm. The secretion system may comprise a single protein or may comprise two or more proteins assembled in a complex e.g. HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In some embodiments, the polypeptide to be secreted include a “secretion tag” of either RNA or peptide origin to direct the polypeptide to specific secretion systems. In some embodiments, the secretion system is able to remove this tag before secreting the polypeptide from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the lysosomal enzyme(s) into the extracellular milieu. In some embodiments, the secretion system involves the generation of a “leaky” or de-stabilized outer membrane, which may be accomplished by deleting or mutagenizing genes responsible for tethering the outer membrane to the rigid peptidoglycan skeleton, including for example, lpp, ompC, ompA, ompF, tolA, tolB, pal, degS, degP, and nlpI. Lpp functions as the primary ‘staple’ of the bacterial cell wall to the peptidoglycan. TolA-PAL and OmpA complexes function similarly to Lpp and are other deletion targets to generate a leaky phenotype. Additionally, leaky phenotypes have been observed when periplasmic proteases, such as degS, degP or nIpI, are deactivated. Thus, in some embodiments, the engineered bacteria have one or more deleted or mutated membrane genes, e.g., selected from lpp, ompA, ompA, ompF, tolA, tolB, and pal genes. In some embodiments, the engineered bacteria have one or more deleted or mutated periplasmic protease genes, e.g., selected from degS, degP, and nlpI. In some embodiments, the engineered bacteria have one or more deleted or mutated gene(s), selected from lpp, ompA, ompA, ompF, tolA, tolB, pal, degS, degP, and nlpI genes.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Recombinant Bacteria

The genetically engineered microorganisms, or programmed microorganisms, such as genetically engineered bacteria of the disclosure are capable of producing one or more enzymes for metabolizing a branched amino acid and/or a metabolite thereof. In some aspects, the disclosure provides a bacterial cell that comprises one or more heterologous gene sequence(s) encoding a branched chain amino acid catabolic enzyme or other protein that results in a decrease in BCAA levels.

In certain embodiments, the genetically engineered bacteria are obligate anaerobic bacteria. In certain embodiments, the genetically engineered bacteria are facultative anaerobic bacteria. In certain embodiments, the genetically engineered bacteria are aerobic bacteria. In some embodiments, the genetically engineered bacteria are Gram-positive bacteria. In some embodiments, the genetically engineered bacteria are Gram-positive bacteria and lack LPS. In some embodiments, the genetically engineered bacteria are Gram-negative bacteria. In some embodiments, the genetically engineered bacteria are Gram-positive and obligate anaerobic bacteria. In some embodiments, the genetically engineered bacteria are Gram-positive and facultative anaerobic bacteria. In some embodiments, the genetically engineered bacteria are non-pathogenic bacteria. In some embodiments, the genetically engineered bacteria are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the genetically engineered bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the genetically engineered bacteria are selected from Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli, Escherichia coli Nissle, Lactobacillus acidophilus, Lactobacillus plantarum, Lactobacillus reuteri, and Lactococcus lactis bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell.

In some embodiments, the genetically engineered bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

One of ordinary skill in the art would appreciate that the genetic modifications disclosed herein may be adapted for other species, strains, and subtypes of bacteria. Furthermore, genes from one or more different species can be introduced into one another, e.g., the kivD gene from Lactococcus lactis (SEQ ID NO: 1) can be expressed in Escherichia coli. In one embodiment, the recombinant bacterial cell does not colonize the subject having the disorder. Unmodified E. coli Nissle and the genetically engineered bacteria of the invention may be destroyed, e.g., by defense factors in the gut or blood serum (Sonnenborn et al., 2009). In some embodiments, the residence time is calculated for a human subject. In some embodiments, residence time in vivo is calculated for the genetically engineered bacteria of the invention.

In some embodiments, the bacterial cell is a genetically engineered bacterial cell. In another embodiment, the bacterial cell is a recombinant bacterial cell. In some embodiments, the disclosure comprises a colony of bacterial cells disclosed herein.

In another aspect, the disclosure provides a recombinant bacterial culture which comprises bacterial cells disclosed herein. In one aspect, the disclosure provides a recombinant bacterial culture which reduces levels of a branched chain amino acid, e.g., leucine, in the media of the culture. In one embodiment, the levels of the branched chain amino acid, e.g., leucine, are reduced by about 50%, about 75%, or about 100% in the media of the cell culture. In another embodiment, the levels of the branched chain amino acid, e.g., leucine, are reduced by about two-fold, three-fold, four-fold, five-fold, six-fold, seven-fold, eight-fold, nine-fold, or ten-fold in the media of the cell culture. In one embodiment, the levels of the branched chain amino acid, e.g., leucine, are reduced below the limit of detection in the media of the cell culture.

In some embodiments of the above described genetically engineered bacteria, the gene encoding a branched chain amino acid catabolism enzyme is present on a plasmid in the bacterium and operatively linked on the plasmid to a promoter that is induced under low-oxygen or anaerobic conditions, such as any of the promoters disclosed herein. In other embodiments, the gene encoding a branched chain amino acid catabolism enzyme is present in the bacterial chromosome and is operatively linked in the chromosome to the promoter that is induced under low-oxygen or anaerobic conditions, such as any of the promoters disclosed herein. In some embodiments of the above described genetically engineered bacteria, the gene encoding a branched chain amino acid catabolic enzyme is present on a plasmid in the bacterium and operatively linked on the plasmid to the promoter that is induced under inflammatory conditions, such as any of the promoters disclosed herein. In other embodiments, the gene encoding a branched chain amino acid catabolic enzyme is present in the bacterial chromosome and is operatively linked in the chromosome to the promoter that is induced under inflammatory conditions, such as any of the promoters disclosed herein.

In some embodiments, the genetically engineered bacteria comprising gene sequence encoding a branched chain amino acid catabolic enzyme is an auxotroph. In one embodiment, the genetically engineered bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the engineered bacteria have more than one auxotrophy, for example, they may be a ΔthyA and ΔdapA auxotroph. In some embodiments, the genetically engineered bacteria comprising gene sequence encoding a branched chain amino acid catabolic enzyme lacks functional ilvC gene sequence, e.g., is a ilvC auxotroph. IlvC encodes keto acid reductoisomerase, which enzyme is required for BCAA synthesis. Knock out of ilvC creates an auxotroph and requires the bacterial cell to import isoleucine and valine to survive.

In some embodiments, the genetically engineered bacteria comprising gene sequence encoding a branched chain amino acid catabolism enzyme further comprise gene sequence(s) encoding a BCAA transporter or other amino acid transporter that transports one or more BCAA(s) into the bacterial cell, for example a transporter that is capable of transporting leucine, valine, and/or isoleucine into a bacterial cell. In certain embodiments, the BCAA transporter is a leucine transporter, e.g., a high-affinity leucine transporter. In certain embodiments, the bacterial cell contains gene sequence encoding livK, livH, livM, livG, and livF genes. In certain embodiments, the BCAA transporter is a BCAA transporter, e.g., a low affinity BCAA transporter. In certain embodiments, the bacterial cell contains gene sequence encoding brnQ gene.

In some embodiments, the genetically engineered bacteria comprising gene sequence encoding a branched chain amino acid catabolism enzyme further comprise gene sequence(s) encoding a secretion protein or protein complex for secreting a biomolecule, such as any of the secretion systems disclosed herein.

In some embodiments, the genetically engineered bacteria comprising gene sequence encoding a branched chain amino acid catabolism enzyme further comprise gene sequence(s) encoding one or more antibiotic gene(s), such as any of the antibiotic genes disclosed herein.

In some embodiments, the genetically engineered bacteria comprising a branched chain amino acid catabolism enzyme further comprise a kill-switch circuit, such as any of the kill-switch circuits provided herein. For example, in some embodiments, the genetically engineered bacteria further comprise one or more genes encoding one or more recombinase(s) under the control of an inducible promoter, and an inverted toxin sequence. In some embodiments, the genetically engineered bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the genetically engineered bacteria further comprise one or more genes encoding an antitoxin. In some embodiments, the engineered bacteria further comprise one or more genes encoding a toxin under the control of a promoter having a TetR repressor binding site and a gene encoding the TetR under the control of an inducible promoter that is induced by arabinose, such as P_{araBAD}. In some embodiments, the genetically engineered bacteria further comprise one or more genes encoding an antitoxin.

In some embodiments, the genetically engineered bacteria are an auxotroph comprising gene sequence encoding a branched chain amino acid catabolism enzyme and further comprises a kill-switch circuit, such as any of the kill-switch circuits described herein.

In some embodiments of the above described genetically engineered bacteria, the gene encoding a branched chain amino acid catabolism enzyme is present on a plasmid in the bacterium. In some embodiments, the gene encoding a branched chain amino acid catabolism enzyme is present in the bacterial chromosome. In some embodiments, the gene sequence(s) encoding a BCAA transporter or other amino acid transporter that transports one or more BCAA(s) into the bacterial cell, for example a transporter that is capable of transporting leucine, valine, and/or isoleucine into a bacterial cell, is present on a plasmid in the bacterium. In some embodiments, the gene sequence(s) encoding a BCAA transporter or other amino acid transporter that transports one or more BCAA(s) into the bacterial cell, for example a transporter that is capable of transporting leucine, valine, and/or isoleucine into a bacterial cell, is present in the bacterial chromosome. In some embodiments, the gene sequence encoding a secretion protein or protein complex for secreting a biomolecule, such as any of the secretion systems disclosed herein, is present on a plasmid in the bacterium. In some embodiments, the gene sequence encoding a secretion protein or protein complex for secreting a biomolecule, such as any of the secretion systems disclosed herein, is present in the bacterial chromosome. In some embodiments, the gene sequence(s) encoding an antibiotic resistance gene is present on a plasmid in the bacterium. In some embodiments, the gene sequence(s) encoding an antibiotic resistance gene is present in the bacterial chromosome.

Branched Chain Amino Acid Catabolism Enzymes

As used herein, the term “branched chain amino acid catabolic or catabolism enzyme” refers to an enzyme involved in the catabolism of a branched chain amino acid to its corresponding α-keto acid counterpart; or the catabolism of an alpha-keto acid to its corresponding aldehyde, acyl-CoA, alcohol, carboxylic acid, or other metabolite counterpart. In some embodiments, the present disclosure provides an engineered bacterium comprising gene sequence(s) encoding one or more branched chain amino acid catabolism enzyme(s). In some embodiments, the branched chain amino acid catabolism enzyme is used to convert a branched chain amino acid, e.g., leucine, valine, isoleucine, to its corresponding α-keto-acid, e.g., α-ketoisocaproate, α-keto-β-methylvalerate, and α-ketoisovalerate. In some embodiments, wherein a branched chain amino acid catabolism enzyme is used to convert a branched chain amino acid, e.g., leucine, valine, isoleucine, to its corresponding α-keto-acid, the engineered bacteria further comprise one or more branched chain amino acid catabolism enzyme(s) to convert an α-keto-acid to its corresponding acetyl CoA, e.g., isovaleryl-CoA, α-methylbutyryl-CoA, and isobutyryl-CoA. In some embodiments, wherein a branched chain amino acid catabolism enzyme is used to convert a branched chain amino acid, e.g., leucine, valine, isoleucine, to its corresponding α-keto-acid, the engineered bacteria further comprise one or more branched chain amino acid catabolism enzyme(s) to convert an α-keto-acid to its corresponding aldehyde, e.g., isovaleraldehyde, isobutyraldehyde, and 2-methylbutyraldehyde. In some embodiments, the engineered bacteria may further comprise an alcohol dehydrogenase enzyme in order to convert the branched chain amino acid-derived aldehyde (e.g., isovaleraldehyde, isobutyraldehyde, 2-methylbutyraldehyde) to its respective alcohol. In some embodiments, the engineered bacteria may further comprise an aldehyde dehydrogenase enzyme in order to convert the branched chain amino acid-derived aldehyde (e.g., isovaleraldehyde, isobutyraldehyde, 2-methylbutyraldehyde) to its respective carboxylic acid.

Enzymes involved in the catabolism of branched chain amino acids are well known to those of skill in the art. For example, in bacteria, leucine dehydrogenase (LeuDH), branched chain amino acid transferase (IlvE), amino acid oxidase (also known as amino acid deaminase) (L-AAD), as well as other known enzymes, can be used to convert a BCAA to its corresponding α-keto acid, e.g., ketoisocaproate (KIC), ketoisovalerate (KIV), and ketomethylvalerate (KMV). Leucine dehydrogenases, branched chain amino acid transamination enzymes (EC 2.6.1.42), and L-amino acid deaminases (L-AAD), which oxidatively deaminate branched chain amino acids into their respective alpha-keto acid, are known (Baker et al., Structure, 3(7):693-705, 1995; Peng et al., J. Bact., 139(2):339-45, 1979; and Kline et al., J. Bact., 130(2):951-3, 1977). In bacteria, branched chain keto acid dehydrogenases (“BCKDs”) are enzyme complexes that oxidatively decarboxylate all three branched chain keto acids into their respective acyl-CoA derivatives. Thus, in one embodiment, the branched chain amino acid catabolism enzyme is a branched chain keto acid dehydrogenase (BCKD). Moreover, in mammals, dehydrogenases specific for 2-ketoisovalerate (EC 1.2.4.4) and 2-keto-3-methylvalerate and 2-keto-isocaproate (EC 1.2.4.3) have been identified (see, for example, Massey et al., Bacteriol Rev., 40(1):42-54, 1976). In bacteria, branched chain keto acid dehydrogenases (“BCKDs”) are enzyme complexes that oxidatively decarboxylate all three branched chain keto acids into their respective acyl-CoA derivatives. Also, for example, in bacteria, α-ketoisovalerate decarboxylase (KivD) enzymes are capable of converting α-keto acids into aldehydes (e.g., isovaleraldehyde, isobutyraldehyde, 2-methylbutyraldehyde). Specifically, the α-ketoisovalerate decarboxylase enzyme KivD is capable of metabolizing valine by converting α-ketoisovalerate to isobutyraldehyde (see, for example, de la Plaza et al., FEMS Microbiol. Lett. 2004, 238(2):367-374). KivD is capable of metabolizing leucine by converting α-ketoisocaproate (KIC) to isovaleraldehyde. KivD is also capable of metabolizing isoleucine by converting α-ketomethylvalerate (KMV) to 2-methylbutyraldehyde. In addition, enzymes for converting isovaleraldehyde, isobutyraldehyde, and 2-methylbutyraldehyde to their respective alcohols or carboxylic acids are known and available. For example, alcohol dehydrogenases (e.g., Adh2, YqhD) can convert isovaleraldehyde, isobutyraldehyde, 2-methylbutyraldehyde to isopentanol, isobutanol, and 2-methylbutanol, respectively. Aldehyde dehydrogenases (e.g., PadA) can convert isovaleraldehyde, isobutyraldehyde, 2-methylbutyraldehyde to isovalerate, isobutyrate, and 2-methylbutyrate, respectively.

In some embodiments, the branched chain amino acid catabolism enzyme increases the rate of branched chain amino acid catabolism. In some embodiments, the branched chain amino acid catabolism enzyme decreases the level of one or more branched chain amino acids, e.g., leucine, isoleucine, and/or valine, in a cell, tissue, or organism. In some embodiments, the branched chain amino acid catabolism enzyme decreases the level of alpha-keto acid derived from BCAA in a cell, tissue, or organism. In some embodiments, the branched chain amino acid catabolism enzyme decreases the level of branched chain amino acid as compared to the level of its corresponding alpha-keto acid in a cell, tissue, or organism. In other embodiments, the branched chain amino acid catabolism enzyme increases the level of alpha-keto acid as compared to the level of its corresponding branched chain amino acid in a cell, tissue, or organism. In some embodiments, the branched chain amino acid catabolism enzyme decreases the level of the branched chain amino acid as compared to the level of its corresponding Acyl-CoA derivative in a cell, tissue, or organism. In some embodiments, the branched chain amino acid catabolism enzyme increases the level of the Acyl-CoA derivative as compared to the level of the branched chain amino acid in a cell, tissue, or organism. In some embodiments, the branched chain amino acid catabolism enzyme decreases the level of alpha-keto aldehyde derived from BCAA, e.g., isovaleraldehyde, isobutyraldehyde, and 2-methylbutyraldehyde, in a cell, tissue, or organism. In some embodiments, the branched chain amino acid catabolism enzyme decreases the level of branched chain amino acid as compared to the level of its corresponding alpha-keto aldehyde, e.g., isovaleraldehyde, isobutyraldehyde, and 2-methylbutyraldehyde, in a cell, tissue, or organism. In other embodiments, the branched chain amino acid catabolism enzyme increases the level of alpha-keto aldehyde, e.g., isovaleraldehyde, isobutyraldehyde, and 2-methylbutyraldehyde, as compared to the level of its corresponding branched chain amino acid in a cell, tissue, or organism. In some embodiments, the branched chain amino acid catabolism enzyme decreases the level of a corresponding downstream metabolite, e.g., isovalerate, isobutyrate, 2-methylbutyrate, isopentanol, isobutanol, and 2-methylbutanol, in a cell, tissue, or organism. In some embodiments, the branched chain amino acid catabolism enzyme decreases the level of branched chain amino acid as compared to the level of a corresponding downstream metabolite, e.g., isovalerate, isobutyrate, 2-methylbutyrate, isopentanol, isobutanol, and 2-methylbutanol, in a cell, tissue, or organism. In other embodiments, the branched chain amino acid catabolism enzyme increases the level of a downstream metabolite, e.g., isovalerate, isobutyrate, 2-methylbutyrate, isopentanol, isobutanol, and 2-methylbutanol, as compared to the level of its corresponding branched chain amino acid in a cell, tissue, or organism.

In some embodiments, the branched chain amino acid catabolism enzyme is a leucine catabolism enzyme. In other embodiments, the branched chain amino acid catabolism enzyme is an isoleucine catabolism enzyme. In other embodiments, the branched chain amino acid catabolism enzyme is a valine catabolism enzyme. In some embodiments, the branched chain amino acid catabolism enzyme is involved in the catabolism of leucine, isoleucine, and valine. In another embodiment, the branched chain amino acid catabolism enzyme is involved in the catabolism of leucine and valine, isoleucine and valine, or leucine and isoleucine. In some embodiments, the branched chain amino acid catabolism enzyme converts leucine, isoleucine, and/or valine into its corresponding α-keto acid. In certain specific embodiments, the present disclosure provides an engineered bacterium comprising gene sequence(s) encoding one or more catabolism enzymes selected from leucine dehydrogenase (LeuDH), BCAA aminotransferase (IlvE), and/or amino acid oxidase (L-AAD).

In some embodiments, the branched chain amino acid catabolism enzyme is an alpha-ketoisocaproic acid (MC) catabolism enzyme. In other embodiments, the branched chain amino acid catabolism enzyme is an alpha-ketoisovaleric acid (KIV) catabolism enzyme. In other embodiments, the branched chain amino acid catabolism enzyme is an alpha-keto-beta-methylvaleric acid (KMV) catabolism enzyme. In other embodiments, the branched chain amino acid catabolism enzyme is involved in the catabolism of alpha-ketoisocaproic acid (KIC), alpha-ketoisovaleric acid (MV), and alpha-keto-beta-methylvaleric acid (KMV). In other embodiments, the branched chain amino acid catabolism enzyme is involved in the catabolism of KIC and KIV, KIC and KMV, or KIV and KMV. In some embodiments, the branched chain amino acid catabolism enzyme converts alpha-ketoisocaproic acid (KIC), alpha-ketoisovaleric acid (MV), and/or alpha-keto-beta-methylvaleric acid (KMV) into its corresponding aldehyde, e.g., isovaleraldehyde, isobutyraldehyde, and/or 2-methylbutyraldehyde. In some embodiments, the present disclosure provides an engineered bacterium comprising gene sequence(s) encoding KivD.

In one embodiment, the branched chain amino acid catabolism enzyme is an isovaleraldehyde catabolism enzyme. In another embodiment, the branched chain amino acid catabolism enzyme is an isobutyraldehyde catabolism enzyme. In another embodiment, the branched chain amino acid catabolism enzyme is 2-methylbutyraldehyde catabolism enzyme. In another embodiment, the branched chain amino acid catabolism enzyme is involved in the catabolism of isovaleraldehyde, isobutyraldehyde, and 2-methylbutyraldehyde. In another embodiment, the branched chain amino acid catabolism enzyme is involved in the catabolism of isovaleraldehyde and isobutyraldehyde, isovaleraldehyde and 2-methylbutyraldehyde, or isobutyraldehyde and 2-methylbutyraldehyde. In some embodiments, the present disclosure provides an engineered bacterium comprising gene sequence(s) encoding one or more alcohol dehydrogenase(s), e.g., Ahd2, YqhD. In some embodiments, the present disclosure provides an engineered bacterium comprising gene sequence(s) encoding one or more aldehyde dehydrogenase(s), e.g., PadA. In some embodiments, the present disclosure provides an engineered bacterium comprising gene sequence(s) encoding one or more alcohol dehydrogenase(s), e.g., Ahd2, YqhD and one or more aldehyde dehydrogenase(s), e.g., PadA.

In some embodiments, the present disclosure provides an engineered bacterium comprising gene sequence(s) encoding one or more branched chain amino acid catabolism enzyme(s) involved in the catabolism of leucine, isoleucine, and/or valine, and further comprises gene sequence(s) encoding one or more branched chain amino acid catabolism enzyme(s) involved in the catabolism of KIC, KIV, and/or KMV. In some embodiments, the present disclosure provides an engineered bacteria comprising gene sequence(s) encoding one or more branched chain amino acid catabolism enzyme(s) involved in the catabolism of leucine, isoleucine, and/or valine, further comprises gene sequence(s) encoding one or more branched chain amino acid catabolism enzyme(s) involved in the catabolism of KIC, KIV, and/or KMV, and further comprises gene sequence(s) encoding one or more branched chain amino acid catabolism enzyme(s) involved in the catabolism of. isovaleraldehyde, isobutyraldehyde, and/or 2-methylbutyraldehyde. In some embodiments, the present disclosure provides an engineered bacterium comprising gene sequence(s) encoding one or more branched chain amino acid catabolism enzyme(s) selected from LeuDH, IlvE, L-AAD, KivD, PadA, Adh2, and YqhD.

In some embodiments, the present disclosure provides an engineered bacterium comprising gene sequence(s) encoding one or more branched chain amino acid catabolism enzyme(s) involved in the conversion of leucine, isoleucine, and/or valine to KIC, KIV, and/or KMV, respectively. In some embodiments, the present disclosure provides an engineered bacterium comprising gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively. In some embodiments, the present disclosure provides an engineered bacterium comprising gene sequence(s) encoding one or more branched chain amino acid catabolism enzyme(s) involved in the conversion of isovaleraldehyde, isobutyraldehyde, and/or 2-methylbutyraldehyde to isovalerate, isobutyrate, and/or 2-methylbutyrate, respectively. In some embodiments, the present disclosure provides an engineered bacterium comprising gene sequence(s) encoding one or more branched chain amino acid catabolism enzyme(s) involved in the conversion of. isovaleraldehyde, isobutyraldehyde, and/or 2-methylbutyraldehyde to isopentanol, isobutanol, and/or 2-methylbutanol respectively.

In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively, and further comprises gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively. In some embodiments, the present disclosure provides an engineered bacterium comprising gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of. isovaleraldehyde, isobutyraldehyde, and/or 2-methylbutyraldehyde to isovalerate, isobutyrate, and/or 2-methylbutyrate, respectively. In some embodiments, the present disclosure provides an engineered bacteria comprising gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of isovaleraldehyde, isobutyraldehyde, and/or 2-methylbutyraldehyde to isopentanol, isobutanol, and/or 2-methylbutanol respectively.

In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively, further comprises gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of isovaleraldehyde, isobutyraldehyde, and/or 2-methylbutyraldehyde to isovalerate, isobutyrate, and/or 2-methylbutyrate, respectively. In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively, further comprises gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of. isovaleraldehyde, isobutyraldehyde, and/or 2-methylbutyraldehyde to isopentanol, isobutanol, and/or 2-methylbutanol, respectively. In some embodiments, the present disclosure provides an engineered bacterium comprising gene sequence(s) encoding one or more branched chain amino acid catabolism enzyme(s) selected from LeuDH, IlvE, and/or L-AAD, KivD, PadA, Adh2, and YqhD.

Enzymes involved in the catabolism of a branched chain amino acid may be expressed or modified in the bacteria disclosed herein to enhance catabolism of a branched chain amino acid, e.g., leucine. Specifically, when a branched chain amino acid catabolism enzyme is expressed in the engineered bacteria disclosed herein, the engineered bacteria are able to convert (deaminate) more branched chain amino acids (e.g., leucine, valine, isoleucine) into their respective alpha-keto acids (KIC, KIV, KMV) and/or convert more BCAA alpha-keto acids (e.g., KIC, KIV, KMV) into respective BCAA-derived aldehydes (e.g., isovaleraldehyde, isobutyraldehyde, 2-methylbutyraldehyde) and/or convert more BCAA-derived aldehydes into respective alcohols (e.g., isopentanol, isobutanol, 2-methylbutanol) and/or convert more BCAA-derived aldehydes into respective carboxylic acids (isovalerate, isobutyrate, 2-methylbutyrate), and/or convert (decarboxylate) more branched chain alpha-keto acids into their respective acyl-CoA derivatives when the catabolism enzyme(s) is expressed, in comparison with unmodified bacteria of the same bacterial subtype under the same conditions. Thus, for example, the genetically engineered bacteria comprising gene sequence encoding a branched chain amino acid catabolism enzyme can catabolize the branched chain amino acid, e.g., leucine, and/or its corresponding alpha-keto acid, e.g., alpha-ketoisocaproate, to treat diseases associated with catabolism of branched chain amino acids, such as Maple Syrup Urine Disease (MSUD) and others described herein.

In some embodiments, the engineered bacteria comprise gene sequence(s) encoding one or more branched chain amino acid catabolism enzyme(s) and gene sequence(s) encoding one or more transporter(s) capable of importing a BCAA or metabolite thereof. In some embodiments, the engineered bacteria comprise gene sequence(s) encoding at least one branched chain amino acid catabolism enzyme and gene sequence(s) encoding two or more copies of a transporter capable of importing a BCAA or metabolite thereof. In some embodiments, the engineered bacteria comprise gene sequence(s) encoding at least one branched chain amino acid catabolism enzyme and gene sequence(s) encoding two or more different transporter(s) capable of importing a BCAA or metabolite thereof. In certain embodiments, the transporter is a leucine transporter. In certain embodiments, the transporter is a valine transporter. In certain embodiments, the transporter is an isoleucine transporter. In certain embodiments, the transporter is a branched chain amino acid transporter, e.g., capable of importing leucine, isoleucine, and valine. In certain specific embodiments, the transporter is selected from LivKHMGF and BrnQ.

In some embodiments, the engineered bacteria comprise gene sequence(s) encoding at least one branched chain amino acid catabolism enzyme and gene sequence(s) encoding one or more BCAA binding proteins, e.g., a BCAA binding protein that assists in bringing BCAA(s) into the bacterial cell. In some embodiments, the engineered bacteria comprise gene sequence(s) encoding at least one branched chain amino acid catabolism enzyme, gene sequence(s) encoding one or more transporter(s) capable of importing one or more BCAAs, and gene sequence(s) encoding one or more BCAA binding proteins, e.g., a BCAA binding protein that assists in bringing BCAA(s) into the bacterial cell. In any of these embodiments, the engineered bacteria comprise gene sequence(s) encoding two or more copies of a BCAA binding protein. In any of these embodiments, the engineered bacteria comprise gene sequence(s) encoding two or more different BCAA binding proteins. In certain embodiments, the BCAA binding protein is LivJ.

In any of the embodiments described above and herein, the engineered bacteria may further comprise one or more genetic modification(s) that reduces export of a branched chain amino acid from the bacteria, e.g., a deletion or mutation in at least one gene associated with the export of a BCAA, e.g., deletion or mutation in leuE gene and/or its promoter (which reduces or eliminates the export of leucine). In some embodiments, the engineered bacteria comprise gene sequence(s) encoding at least one branched chain amino acid catabolism enzyme and at least one genetic modification that reduces export of a branched chain amino acid. In certain specific embodiments, the engineered bacteria comprise gene sequence(s) encoding at least one branched chain amino acid catabolism enzyme, gene sequence(s) encoding one or more transporter(s) capable of importing a BCAA, and at least one genetic modification that reduces export of a branched chain amino acid. In certain specific embodiments, the engineered bacteria comprise gene sequence(s) encoding at least one branched chain amino acid catabolism enzyme, gene sequence(s) encoding one or more transporter(s) capable of importing a BCAA, gene sequence(s) encoding one or more BCAA binding proteins, and at least one genetic modification that reduces export of a branched chain amino acid. In some embodiments, the engineered bacteria comprise gene sequence(s) encoding at least one branched chain amino acid catabolism enzyme, gene sequence(s) encoding one or more BCAA binding proteins, and at least one genetic modification that reduces export of a branched chain amino acid. In any of these embodiments, the genetic modification may be a deletion or mutation in one or more gene(s) that allow or assist in the export of a BCAA. In any of these embodiment, the genetic modification may be a deletion or mutation in a leuE gene and/or its promoter.

In any of the embodiments described above and herein, the engineered bacteria comprise gene sequence(s) encoding one or more branched chain amino acid catabolism enzyme(s), and at least one genetic modification that reduces endogenous biosynthesis of a branched chain amino acid, for example, a deletion or mutation in at least one gene required for BCAA synthesis, e.g., deletion or mutation in ilvC gene and/or its promoter, which gene is required for BCAA synthesis and whose absence creates an auxotroph requiring the bacterial cell to import leucine. In some embodiments, the engineered bacteria comprise gene sequence(s) encoding one or more branched chain amino acid catabolism enzyme(s), gene sequence(s) encoding one or more transporter(s) capable of importing a BCAA, and at least one genetic modification that reduces endogenous biosynthesis of a branched chain amino acid, for example, a deletion or mutation in at least one gene required for BCAA synthesis. In some embodiments, the engineered bacteria comprise gene sequence(s) encoding at least one branched chain amino acid catabolism enzyme, gene sequence(s) encoding one or more BCAA binding proteins, and at least one genetic modification that reduces endogenous biosynthesis of a branched chain amino acid. In some embodiments, the engineered bacteria comprise gene sequence(s) encoding at least one branched chain amino acid catabolism enzyme, at least one genetic modification that reduces export of a branched chain amino acid, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more transporter(s) capable of importing a BCAA, gene sequence(s) encoding one or more BCAA binding proteins, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more transporter(s) capable of importing a BCAA, at least one genetic modification that reduces export of a branched chain amino acid, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more BCAA binding proteins, at least one genetic modification that reduces export of a branched chain amino acid, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more transporter(s) capable of importing a BCAA, gene sequence(s) encoding one or more BCAA binding proteins, at least one genetic modification that reduces export of a branched chain amino acid, and at least one genetic modification that reduces endogenous biosynthesis of a branched chain amino acid. In any of these embodiments, the at least one genetic modification that reduces endogenous biosynthesis of a branched chain amino acid can be a deletion or mutation in at least one gene required for BCAA synthesis, e.g., deletion or mutation in ilvC gene and/or its promoter.

In any of the embodiments described above and herein, the gene sequence(s) encoding at least one branched chain amino acid catabolism enzyme, and/or gene sequence(s) encoding one or more transporter(s) capable of importing a BCAA, and/or gene sequence(s) encoding one or more BCAA binding proteins, and/or other sequence can be present in the bacterial chromosome. In any of the embodiments described above and herein, the gene sequence(s) encoding at least one branched chain amino acid catabolism enzyme, and/or gene sequence(s) encoding one or more transporter(s) capable of importing a BCAA, and/or gene sequence(s) encoding one or more BCAA binding proteins, and/or other sequence can be present in one or more plasmids.

In some embodiments, the engineered bacteria comprise gene sequence(s) encoding one or more branched chain amino acid catabolism enzyme(s) in which the one or more enzymes are from a different organism, e.g., a different species of bacteria. In some embodiments, the engineered bacteria comprise gene sequence(s) encoding one or more branched chain amino acid catabolism enzyme(s) in which the one or more enzymes are native to the bacterium. In some embodiments, the engineered bacteria comprise gene sequence(s) encoding one or more branched chain amino acid catabolism enzyme(s) in which one or more of the enzymes are native and one or more of the enzymes are from a different organism, e.g., a different species of bacteria. In other embodiments, the bacterial cell comprises more than one copy of a native gene encoding a branched chain amino acid catabolism enzyme. In other embodiments, the bacterial cell comprises more than one copy of a non-native gene encoding a branched chain amino acid catabolism enzyme. In other embodiments, the bacterial cell comprises at least one, two, three, four, five, six or more copies of a gene encoding a branched chain amino acid catabolism enzyme, which can be native or non-native. In other embodiments, the bacterial cell comprises multiple copies of a gene encoding a branched chain amino acid catabolism enzyme. In some embodiments, the bacterial cell comprises gene sequence(s) encoding multiple copies of two or more different branched chain amino acid catabolism enzymes.

In some embodiments, the engineered bacteria comprise gene sequence(s) encoding one or more branched chain amino acid transporters in which the one or more transporters are from a different organism, e.g., a different species of bacteria. In some embodiments, the engineered bacteria comprise gene sequence(s) encoding one or more branched chain amino acid transporter(s) in which the one or more transporters are native to the bacterium. In some embodiments, the engineered bacteria comprise gene sequence(s) encoding one or more branched chain amino acid transporters(s) in which one or more of the transporters are native and one or more of the transporters are from a different organism, e.g., a different species of bacteria. In other embodiments, the bacterial cell comprises more than one copy of a native gene encoding a branched chain amino acid transporter. In other embodiments, the bacterial cell comprises more than one copy of a non-native gene encoding a branched chain amino acid transporter. In other embodiments, the bacterial cell comprises at least one, two, three, four, five, six or more copies of a gene encoding a branched chain amino acid transporter, which can be native or non-native. In other embodiments, the bacterial cell comprises multiple copies of a gene encoding a branched chain amino acid transporters. In some embodiments, the bacterial cell comprises gene sequence(s) encoding multiple copies of two or more different branched chain amino acid transporters.

In some embodiments, the engineered bacteria comprise gene sequence(s) encoding one or more branched chain amino acid binding protein(s) in which the one or more binding protein(s) are from a different organism, e.g., a different species of bacteria. In some embodiments, the engineered bacteria comprise gene sequence(s) encoding one or more branched chain amino acid binding protein(s) in which the one or more binding protein(s) are native to the bacterium. In some embodiments, the engineered bacteria comprise gene sequence(s) encoding one or more branched chain amino acid binding protein(s) in which one or more of the binding protein(s) are native and one or more of the binding protein(s) are from a different organism, e.g., a different species of bacteria. In other embodiments, the bacterial cell comprises more than one copy of a native gene encoding a branched chain amino acid binding protein. In other embodiments, the bacterial cell comprises more than one copy of a non-native gene encoding a branched chain amino acid binding protein. In other embodiments, the bacterial cell comprises at least one, two, three, four, five, six or more copies of a gene encoding a branched chain amino acid binding protein, which can be native or non-native. In other embodiments, the bacterial cell comprises multiple copies of a gene encoding a branched chain amino acid binding protein. In some embodiments, the bacterial cell comprises gene sequence(s) encoding multiple copies of two or more different branched chain amino acid binding proteins.

Branched chain amino acid catabolism enzymes are known in the art. In some embodiments, the branched chain amino acid catabolism enzyme is encoded by a gene encoding a branched chain amino acid catabolism enzyme derived from a bacterial species. In some embodiments, a branched chain amino acid catabolism enzyme is encoded by a gene encoding a branched chain amino acid catabolism enzyme derived from a non-bacterial species. In some embodiments, a branched chain amino acid catabolism enzyme is encoded by a gene derived from a eukaryotic species, e.g., a yeast species or a plant species. In one embodiment, a branched chain amino acid catabolism enzyme is encoded by a gene derived from a mammalian species, e.g., a human. In some embodiments, the engineered bacteria comprise gene sequence(s) encoding at least one branched chain amino acid catabolism enzyme derived from a bacterial species and at least one branched chain amino acid catabolism enzyme derived from a non-bacterial species. In one embodiment, the gene encoding the branched chain amino acid catabolism enzyme is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Pseudomonas, Psychrobacter, Ralstonia, Saccharomyces, Salmonella, Sarcina, Serratia, Staphylococcus, and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Pseudomonas aeruginosa, Psychrobacter anticus, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Yersinia enterocolitica, Yersinia mollaretii, Yersinia kristensenii, Yersinia rohdei, and Yersinia aldovae.

In some embodiments, the gene encoding a branched chain amino acid catabolism enzyme, BCAA transporter, BCAA binding protein, and/or other sequence has been codon-optimized for use in the host organism. In one embodiment, the gene encoding a branched chain amino acid catabolism enzyme has been codon-optimized for use in Escherichia coli. Examples of codon-optimized sequences include SEQ ID NO:29, SEQ ID NO:32, SEQ ID NO:39, and SEQ ID NO:41.

In some embodiments, the branched chain amino acid catabolism enzyme converts a branched chain amino acid to its corresponding alpha-keto acid. Such enzymes include, for example, LeuDH (SEQ ID NOs:19 and 20), IlvE (SEQ ID NO:s 21 and 22), L-AAD (SEQ ID NOs: 23-26). In other embodiments, the branched chain amino acid catabolism enzyme converts a branched chain keto acid to its corresponding aldehyde. For example, in some embodiments, the branched chain amino acid catabolism enzyme is an α-ketoisovalerate decarboxylase (KivD) (SEQ ID NO:27, 28, and 29). In other embodiments, the branched chain amino acid catabolism enzyme converts a branched chain keto acid to its corresponding acetyl-CoA. For example, in some embodiments, the branched chain amino acid catabolism enzyme is a branched chain keto acid dehydrogenase (BCKD). In some embodiments, the branched chain amino acid catabolism enzyme is a branched chain amino acid deaminase, such as an amino acid dehydrogenase, or a branched chain amino acid aminotransferase. In some embodiments, the branched chain amino acid catabolism enzyme is KdcA (SEQ ID NOs:30, 31, and 32). In other embodiments, the branched chain amino acid catabolism enzyme is THI3/KID1 (SEQ ID NOs:33 and 34). In other embodiments, the branched chain amino acid catabolism enzyme is ARO10 (SEQ ID NOs:35 and 36).

In other embodiments, the branched chain amino acid catabolism enzyme converts an aldehyde into its corresponding alcohol. For example, the branched chain amino acid catabolism enzyme may be an alcohol dehydrogenase. In one embodiment, the alcohol dehydrogenase is Adh2 (SEQ ID NOs: 37, 38, and 39). In another embodiment, the alcohol dehydrogenase is Adh6 (SEQ ID NOs: 40 and 41). In another embodiment, the alcohol dehydrogenase is Adh1 (SEQ ID NOs: 42 and 43). In another embodiment, the alcohol dehydrogenase is Adh3 (SEQ ID NOs: 44 and 45). In another embodiment, the alcohol dehydrogenase is Adh4 (SEQ ID NOs:46 and 47). In another embodiment, the alcohol dehydrogenase is SFA1 (SEQ ID NOs:52 and 53). In another embodiment, the alcohol dehydrogenase is YqhD (SEQ ID NOs: 60 and 61) In other embodiments, the branched chain amino acid catabolism enzyme converts an aldehyde into its corresponding carboxylic acid. For example, the branched chain amino acid catabolism enzyme may be an aldehyde dehydrogenase. In one embodiment, the aldehyde dehydrogenase is PadA (SEQ ID NOs: 62 and 63).

In some embodiments, the engineered bacteria comprise gene sequence(s) encoding one or more transporter(s) capable of importing a BCAA or metabolite thereof. In certain embodiments, the transporter is a leucine transporter. In certain embodiments, the transporter is a valine transporter. In certain embodiments, the transporter is an isoleucine transporter. In certain embodiments, the transporter is a branched chain amino acid transporter, e.g., capable of importing leucine, isoleucine, and valine. The term “BCAA transporter” is meant to refer to a transporter that specifically transports leucine, isoleucine, or valine, and also to a transporter that is able to transport any BCAA, including for example, the ability to transport leucine, isoleucine, and valine. For example, in some embodiments, the transporter is LivKHMGF (as comprised in SEQ ID NOs: 5, 7, and 10). In some embodiments, the transporter is BrnQ (SEQ ID Nos: 64 and 65).

In some embodiments, the engineered bacteria comprise gene sequence(s) encoding one or more BCAA binding proteins, e.g., a BCAA binding protein that assists in bringing BCAA(s) into the bacterial cell. For example, in some embodiments, the BCAA binding protein is LivJ (SEQ ID NO: 12).

The present disclosure further comprises genes encoding functional fragments of a branched chain amino acid catabolism enzyme, BCAA transporter, BCAA binding protein, and/or other sequence or functional variants of a branched chain amino acid catabolism enzyme, BCAA transporter, BCAA binding protein, and/or other sequence. As used herein, the term “functional fragment thereof” or “functional variant thereof” of a branched chain amino acid catabolism enzyme, BCAA transporter, BCAA binding protein, and/or other sequence refers to fragment or variant sequence having qualitative biological activity in common with the wild-type branched chain amino acid catabolism enzyme, BCAA transporter, BCAA binding protein, and/or other sequence from which the fragment or variant was derived. For example, a functional fragment or a functional variant of a mutated branched chain amino acid catabolism enzyme is one which retains essentially the same ability to catabolize a branched chain amino acid and/or its corresponding alpha-keto acid or aldehyde or other metabolite as the branched chain amino acid catabolism enzyme from which the functional fragment or functional variant was derived. For example, a polypeptide having branched chain amino acid catabolism enzyme activity may be truncated at the N-terminus or C-terminus and the retention of branched chain amino acid catabolism enzyme activity assessed using assays known to those of skill in the art, including the exemplary assays provided herein. In one embodiment, the recombinant bacterial cell disclosed herein comprises a heterologous gene encoding a branched chain amino acid catabolism enzyme functional variant. In another embodiment, the recombinant bacterial cell disclosed herein comprises a heterologous gene encoding a branched chain amino acid catabolism enzyme functional fragment.

The present disclosure encompasses genes encoding a branched chain amino acid catabolism enzyme, BCAA transporter, BCAA binding protein, and/or other sequence comprising amino acids in its sequence that are substantially the same as an amino acid sequence described herein Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Be, Asn, Gln, Phe, Cys, Pro, Trp, Tyr, Val).

The present disclosure encompasses branched chain amino acid catabolism enzymes, BCAA transporters, BCAA binding proteins, and/or other sequences which have a certain percent identity to a gene or protein sequence described herein. For example, the disclosure encompasses branched chain amino acid catabolism enzymes, BCAA transporters, BCAA binding proteins, and/or other sequences having at least 60%, 65%, 70%, 75%, 80%, 85%, 90%, 91%, 92%, 93%, 94%, 95%, 96%, 97%, 98%, 99%, or 100% identity to a nucleic acid sequence or amino acid sequence disclosed herein. As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

Assays for testing the activity of a branched chain amino acid catabolism enzyme, BCAA transporter, BCAA binding protein, and/or other sequence, or a functional variant, or a functional fragment thereof are well known to one of ordinary skill in the art. For example, branched chain amino acid catabolism, BCAA transporter, BCAA binding protein, and/or other sequence can be assessed by expressing the protein, functional variant, or fragment thereof, in a recombinant bacterial cell that lacks endogenous branched chain amino acid catabolism enzyme activity. Branched chain amino acid catabolism can be assessed using the coupled enzymatic assay method as described by Zhang et al. (see, for example, Zhang et al., Proc. Natl. Acad. Sci., 105(52):20653-58, 2008). Furthermore, catabolism of branched chain amino acids can also be assessed in vitro by measuring the disappearance of alpha-ketoisovalerate as described by de la Plaza (see, for example, de la Plaza et al., FEMS Microbiol. Letters, 2004, 238(2):367-374). BCAA as well as the branched chain keto acid can be quantified by liquid chromatography tandem mass spectrometry (LC-MS/MS), as described herein.

In some embodiments, the gene encoding a branched chain amino acid catabolism enzyme, BCAA transporter, BCAA binding protein, and/or other sequence is mutagenized; mutants exhibiting increased activity are selected; and the mutagenized gene encoding the branched chain amino acid catabolism enzyme, BCAA transporter, BCAA binding protein, and/or other sequence is isolated and inserted into the bacterial cell. In some embodiments, the gene encoding an α-ketoisovalerate decarboxylase, e.g., kivD, is mutagenized; mutants exhibiting decreased activity are selected; and the mutagenized gene encoding the α-ketoisovalerate decarboxylase, e.g., kivD, is isolated and inserted into the bacterial cell. The gene comprising the modifications described herein may be present on a plasmid or chromosome.

In some embodiments, the gene encoding the branched chain amino acid catabolism enzyme, BCAA transporter, BCAA binding protein, and/or other sequence is directly operably linked to a first promoter. In other embodiments, the gene encoding the branched chain amino acid catabolism enzyme, BCAA transporter, BCAA binding protein, and/or other sequence is indirectly operably linked to a first promoter. In some embodiment, the gene encoding the branched chain amino acid catabolism enzyme, BCAA transporter, BCAA binding protein, and/or other sequence is operably linked to a promoter that is not its native promoter.

In some embodiments, the gene encoding the branched chain amino acid catabolism enzyme, BCAA transporter, BCAA binding protein, and/or other sequence is expressed under the control of a constitutive promote. In other embodiments, the gene encoding the branched chain amino acid catabolism enzyme, BCAA transporter, BCAA binding protein, and/or other sequence is expressed under the control of an inducible promoter. In some embodiments, the gene encoding the branched chain amino acid catabolism enzyme, BCAA transporter, BCAA binding protein, and/or other sequence is expressed under the control of a promoter that is directly or indirectly induced by exogenous environmental conditions. In some embodiments, the gene encoding the branched chain amino acid catabolism enzyme, BCAA transporter, BCAA binding protein, and/or other sequence is expressed under the control of a promoter that is directly or indirectly induced by low-oxygen or anaerobic conditions, wherein expression of the gene encoding the branched chain amino acid catabolism enzyme is activated under low-oxygen or anaerobic environments, such as the environment of the mammalian gut. In some embodiments, the gene encoding the branched chain amino acid catabolism enzyme, BCAA transporter, BCAA binding protein, and/or other sequence is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein. Examples of other inducible promoters are provided herein below.

The gene encoding the branched chain amino acid catabolism enzyme, BCAA transporter, BCAA binding protein, and/or other sequence may be present on a plasmid or chromosome in the bacterial cell. In some embodiments, the gene encoding the branched chain amino acid catabolism enzyme, BCAA transporter, BCAA binding protein, and/or other sequence is located on a plasmid in the bacterial cell. In other embodiments, the gene encoding the branched chain amino acid catabolism enzyme, BCAA transporter, BCAA binding protein, and/or other sequence is located in the chromosome of the bacterial cell. In other embodiments, a native copy of the gene encoding the branched chain amino acid catabolism enzyme, BCAA transporter, BCAA binding protein, and/or other sequence is located in the chromosome of the bacterial cell, and a gene encoding a branched chain amino acid catabolism enzyme, BCAA transporter, BCAA binding protein, and/or other sequence from the same or a different species of bacteria is located on a plasmid in the bacterial cell. In other embodiments, a native copy of the gene encoding the branched chain amino acid catabolism enzyme, BCAA transporter, BCAA binding protein, and/or other sequence is located on a plasmid in the bacterial cell, and a gene encoding the branched chain amino acid catabolism enzyme, BCAA transporter, BCAA binding protein, and/or other sequence from a different species of bacteria is located on a plasmid in the bacterial cell. In other embodiments, a native copy of the gene encoding the branched chain amino acid catabolism enzyme, BCAA transporter, BCAA binding protein, and/or other sequence is located in the chromosome of the bacterial cell, and a gene encoding the branched chain amino acid catabolism enzyme, BCAA transporter, BCAA binding protein, and/or other sequence from a different species of bacteria is located in the chromosome of the bacterial cell.

In some embodiments, the gene encoding the branched chain amino acid catabolism enzyme, BCAA transporter, BCAA binding protein, and/or other sequence is expressed on a low-copy plasmid. In some embodiments, the gene encoding the branched chain amino acid catabolism enzyme, BCAA transporter, BCAA binding protein, and/or other sequence is expressed on a high-copy plasmid. In some embodiments, the high-copy plasmid may be useful for increasing expression of the branched chain amino acid catabolism enzyme, BCAA transporter, BCAA binding protein, and/or other sequence, thereby increasing the catabolism of the branched chain amino acid, e.g., leucine.

In some embodiments, the engineered bacteria convert the branched chain amino acid(s) and/or corresponding alpha-keto acid(s) and/or other corresponding metabolite(s) to a non-toxic or low toxicity metabolite, e.g., isovaleraldehyde, isobutyraldehyde, 2-methylbutyraldehyde, isovaleric acid, isobutyric acid, 2-methylbutyric acid, isopentanol, isobutanol, and 2-methylbutanol. Table 2 chart showing that the products of BCAA degradation by the engineered bacteria have very low oral toxicity.

TABLE 2
Toxicity of BCAA degradation products

| | Oral LD50 | Oral NOAEL* |
|---|---|---|
| | (rat) | (rat) |
| Compound | (mg/kg) | (mg/kg/d) |
| Isovaleraldehyde | 5740 | N/D |
| Isolbutyraldehyde | 3730 | N/D |
| 2-methylbutyraldehyde | 6884 | N/D |
| Isovaleric acid | 2500 | N/D |
| Isobutyric acid | 2230 | N/D |
| 2-metylbutyric acid | 1750 | N/D |
| Isopentanol | >5000 | 1250 |
| Isobutanol | 3350 | >1450 |
| 2-methylbutanol | 4170 | N/D |

*No-Observed-Adverse-Effect

A. Branched Chain Ketoacid Decarboxylase

In one embodiment, the branched chain amino acid catabolism enzyme is a branched chain ketoacid decarboxylase, including but not limited to, KivD. In a non-limiting example, KivD is from Lactococcus lactis. Another non-limiting example is KdcA (e.g., from Lactococcus lactis). Thus, in some embodiments, the engineered bacteria comprise gene sequence(s) encoding one or more copies of a branched chain ketoacid decarboxylase. In some embodiments, the engineered bacteria comprise gene sequence(s) encoding one, two, three, four, five, six, or more copies of a branched chain ketoacid decarboxylase. The one or more copies of a branched chain ketoacid decarboxylase can be one or more copies of the same gene or can be different genes encoding α-ketoisovalerate decarboxylase, e.g., gene(s) from a different species or otherwise having a different gene sequence. The one or more copies of a branched chain ketoacid decarboxylase can be present in the bacterial chromosome or can be present in one or more plasmids. As used herein “α-ketoisovalerate decarboxylase” or “alpha-ketoisovalerate decarboxylase” or “branched-chain α-keto acid decarboxylase” or “α-ketoacid decarboxylase” or “branched chain ketoacid decarboxylase” or “2-ketoisovalerate decarboxylase” (referred to herein also as KivD or ketoisovalerate decarboxylase) refers to any polypeptide having enzymatic activity that catalyzes the conversion of a branched chain alpha-keto acid (BCKA), such as α-ketoisovalerate (2-oxoisopentanoate), α-ketomethylvalerate (3-methyl-2-oxopentanoate), or α-ketoisocaproate 4-methyl-2-oxopentanoate), to its corresponding aldehyde, such as isobutyraldehyde, 2-methylbutyraldehyde, or isovaleraldehyde, and carbon dioxide. Branched chain ketoacid decarboxylase enzymes are available from many microorganism sources, including those disclosed herein. Branched chain ketoacid decarboxylase employs the co-factor thiamine diphosphate (also known as thiamine pyrophosphate or “TPP” or “TDP”). Thiamine is the vitamin form of the co-factor which, when transported into a cell, is converted to thiamine diphosphate. Alpha-ketoisovalerate decarboxylase also employs Mg²⁺. Branched chain ketoacid decarboxylase may be a homotetramer.

The bacterial cells disclosed herein may comprise a heterologous gene encoding a branched chain ketoacid decarboxylase enzyme and are capable of converting α-keto acids into aldehydes. For example, the branched chain ketoacid decarboxylase enzyme KivD is capable of metabolizing leucine (see, for example, de la Plaza et al., FEMS Microbiol. Lett. 2004, 238(2):367-374), and a cytosolically active KivD should generally exhibit the ability to convert ketoisovalerate, ketomethylvalerate, and ketoisocaproate to isobutyraldehyde, 2-methylbutyraldehyde, and isovaleraldehyde.

Multiple distinct a branched chain ketoacid decarboxylase proteins are known in the art (see, e.g., US Pat. Appl. Publ. No. 2013/0203138, the entire contents of which are incorporated herein by reference). In some embodiments, branched chain ketoacid decarboxylase is encoded by a branched chain ketoacid decarboxylase gene derived from a bacterial species. In some embodiments, a branched chain ketoacid decarboxylase is encoded by a branched chain ketoacid decarboxylase gene derived from a non-bacterial species. In some embodiments, a branched chain ketoacid decarboxylase is encoded by a branched chain ketoacid decarboxylase gene derived from a eukaryotic species, e.g., a yeast species or a plant species. In some embodiments, a branched chain ketoacid decarboxylase is encoded by a branched chain ketoacid decarboxylase gene derived from a mammalian species. In one embodiment, the a branched chain ketoacid decarboxylase gene is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Psychrobacter, Ralstonia, Saccharomyces, Salmonella, Sarcina, Serratia, Staphylococcus, and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Psychrobacter anticus, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Yersinia enterocolitica, Yersinia mollaretii, Yersinia kristensenii, Yersinia rohdei, and Yersinia aldovae. In some embodiments, the branched chain ketoacid decarboxylase is encoded by an a branched chain ketoacid decarboxylase gene derived from Lactococcus lactis, e.g., IFPL730. In another embodiment, the branched chain ketoacid decarboxylase, e.g., kivD gene, is derived from Enterobacter cloacae (Accession No. P23234.1), Mycobacterium smegmatis (Accession No. A0R480.1), Mycobacterium tuberculosis (Accession NO. 053865.1), Mycobacterium avium (Accession No. Q742Q2.1), Azospirillum brasilense (Accession No. P51852.1), or Bacillus subtilis (see Oku et al., J. Biol. Chem. 263: 18386-96, 1988).

In one embodiment, the branched chain ketoacid decarboxylase gene has been codon-optimized for use in the recombinant bacterial cell. In one embodiment, the branched chain ketoacid decarboxylase gene has been codon-optimized for use in Escherichia coli. For example, a codon-optimized kivD sequence is set forth as SEQ ID NO: 29.

In one embodiment, the branched chain ketoacid decarboxylase gene is a kivD gene. In another embodiment, the kivD gene is a Lactococcus lactis kivD gene or kivD gene derived from Lactococcus lactis. When a branched chain ketoacid decarboxylase is expressed in the recombinant bacterial cells disclosed herein, the bacterial cells catabolize more branched chain amino acid, e.g., leucine, than unmodified bacteria of the same bacterial subtype under the same conditions (e.g., culture or environmental conditions). Thus, the genetically engineered bacteria comprising a heterologous gene encoding a branched chain ketoacid decarboxylase may be used to catabolize excess branched chain amino acids, e.g., leucine, to treat a disease associated with the catabolism of a branched chain amino acid, including Maple Syrup Urine Disease (MSUD).

The present disclosure further comprises genes encoding functional fragments of a branched chain ketoacid decarboxylase or functional variants of a branched chain ketoacid decarboxylase gene. As used herein, the term “functional fragment thereof” or “functional variant thereof” of a branched chain ketoacid decarboxylase gene relates to a sequence having qualitative biological activity in common with the wild-type branched chain ketoacid decarboxylase from which the fragment or variant was derived. For example, a functional fragment or a functional variant of a mutated branched chain ketoacid decarboxylase protein is one which retains essentially the same ability to catabolize BCKAs as a branched chain ketoacid decarboxylase protein from which the functional fragment or functional variant was derived. For example, a polypeptide having branched chain ketoacid decarboxylase activity may be truncated at the N-terminus or C-terminus and the retention of branched chain ketoacid decarboxylase activity assessed using assays known to those of skill in the art, including the exemplary assays provided herein. In one embodiment, the recombinant bacterial cell disclosed herein comprises a heterologous gene encoding a branched chain ketoacid decarboxylase functional variant. In another embodiment, the recombinant bacterial cell disclosed herein comprises a heterologous gene encoding a branched chain ketoacid decarboxylase functional fragment.

Assays for testing the activity of a branched chain ketoacid decarboxylase, a branched chain ketoacid decarboxylase functional variant, or a branched chain ketoacid decarboxylase functional fragment are well known to one of ordinary skill in the art. For example, branched chain ketoacid decarboxylase activity can be assessed by expressing the protein, functional variant, or fragment thereof, in a recombinant bacterial cell that lacks endogenous branched chain ketoacid decarboxylase activity. Also, branched chain ketoacid decarboxylase activity can be assessed using the coupled enzymatic assay method as described by Zhang et al. (see, for example, Zhang et al., Proc. Natl. Acad. Sci., 105(52):20653-58, 2008). Alpha-ketoisovalerate decarboxylase activity can also be assessed in vitro by measuring the disappearance of alpha-ketoisovalerate as described by de la Plaza (see, for example, de la Plaza et al., FEMS Microbiol. Letters, 2004, 238(2):367-374).

In some embodiments, the gene encoding a branched chain ketoacid decarboxylase, e.g., kivD, is mutagenized; mutants exhibiting increased activity are selected; and the mutagenized gene encoding the α-ketoisovalerate decarboxylase, e.g., kivD, is isolated and inserted into the bacterial cell. The gene comprising the modifications described herein may be present on a plasmid or chromosome.

Accordingly, in some embodiments, the kivD gene has at least about 80% identity with the entire sequence of SEQ ID NO:1. Accordingly, in one embodiment, the kivD gene has at least about 90% identity with the entire sequence of SEQ ID NO:1. Accordingly, in one embodiment, the kivD gene has at least about 95% identity with the entire sequence of SEQ ID NO:1. Accordingly, in one embodiment, the kivD gene has at least about 85%, 86%, 87%, 88%, 89%, 90%, 91%, 92%, 93%, 94%, 95%, 96%, 97%, 98%, or 99% identity with the entire sequence of SEQ ID NO:1. In another embodiment, the kivD gene comprises the sequence of SEQ ID NO:1. In yet another embodiment, the kivD gene consists of the sequence of SEQ ID NO:1.

In other embodiments, the engineered bacteria comprise gene sequence(s) encoding at least one branched chain ketoacid decarboxylase enzyme and further comprise gene sequence encoding one or more polypeptides selected from other branched chain amino acid catabolism enzyme(s), BCAA transporter(s), and BCAA binding protein(s). Thus, in some embodiments, the at least one branched chain ketoacid decarboxylase enzyme is coexpressed with an additional branched chain amino acid catabolism enzyme, e.g., a branched chain amino acid dehydrogenase, amino acid oxidase (also known as amino acid deaminase), and/or aminotransferase. In some embodiments, the at least one α-ketoisovalerate decarboxylase gene is coexpressed with a leucine dehydrogenase, e.g., (leuDH or ldh), described in more detail below. In other embodiments, the at least one branched chain ketoacid decarboxylase gene is coexpressed with a branched chain amino acid aminotransferase, e.g., ilvE, described in more detail below. In other embodiments, the at least one branched chain ketoacid decarboxylase gene is coexpressed with an amino acid deaminase, e.g., L-AAD, described in more detail below. In some embodiments, the engineered bacteria comprise gene sequence(s) encoding at least one branched chain ketoacid decarboxylase enzyme and gene sequence(s) encoding one or more branched chain amino acid dehydrogenase(s) (e.g., leuDH). In some embodiments, the engineered bacteria comprise gene sequence(s) encoding at least one branched chain ketoacid decarboxylase enzyme and gene sequence(s) encoding one or more amino acid oxidase(s) (e.g. L-AAD)). In some embodiments, the engineered bacteria comprise gene sequence(s) encoding at least one branched chain ketoacid decarboxylase enzyme and gene sequence(s) encoding one or more aminotransferase(s) (e.g., ilvE).

In some embodiments, the at least one branched chain ketoacid decarboxylase enzyme is coexpressed with an aldehyde dehydrogenase, e.g., padA, described in more detail below. In some embodiments, the at least one branched chain ketoacid decarboxylase enzyme is coexpressed with an alcohol dehydrogenase, e.g., adh2, yqhD, described in more detail below. In some embodiments, the engineered bacteria comprise gene sequence(s) encoding at least one branched chain ketoacid decarboxylase enzyme and gene sequence(s) encoding one or more aldehyde dehydrogenase(s) (e.g., padA)). In some embodiments, the engineered bacteria comprise gene sequence(s) encoding at least one branched chain ketoacid decarboxylase enzyme and gene sequence(s) encoding one or more alcohol dehydrogenase(s) (e.g., adh2, yqhD).

In some embodiments, the at least one α-ketoisovalerate decarboxylase gene is coexpressed with a leucine dehydrogenase, e.g., (leuDH or ldh) and an aldehyde dehydrogenase, e.g., padA and/or an alcohol dehydrogenase, e.g., adh2, yqhD. In other embodiments, the at least one α-ketoisovalerate decarboxylase gene is coexpressed with a branched chain amino acid aminotransferase, e.g., ilvE and an aldehyde dehydrogenase, e.g., padA and/or an alcohol dehydrogenase, e.g., adh2, yqhD. In other embodiments, the at least one α-ketoisovalerate decarboxylase gene is coexpressed with an amino acid deaminase, e.g., L-AAD and an aldehyde dehydrogenase, e.g., padA and/or an alcohol dehydrogenase, e.g., adh2, yqhD. In some embodiments, the engineered bacteria comprise gene sequence(s) encoding at least one branched chain ketoacid decarboxylase enzyme and gene sequence(s) encoding one or more leucine dehydrogenase(s), e.g., (leuDH), gene sequence encoding one or more aldehyde dehydrogenase(s) (e.g., padA) and/or gene sequence encoding one or more alcohol dehydrogenases (e.g., adh2, yqhD). In some embodiments, the engineered bacteria comprise gene sequence(s) encoding at least one branched chain ketoacid decarboxylase enzyme and gene sequence(s) encoding one or more branched chain amino acid aminotransferase, (e.g., ilvE), gene sequence encoding one or more aldehyde dehydrogenase(s) (e.g., padA) and/or gene sequence encoding one or more alcohol dehydrogenases (e.g., adh2, yqhD). In some embodiments, the engineered bacteria comprise gene sequence(s) encoding at least one branched chain ketoacid decarboxylase enzyme and gene sequence(s) encoding one or more an amino acid deaminase, e.g., L-AAD, gene sequence encoding one or more aldehyde dehydrogenase(s) (e.g., padA) and/or gene sequence encoding one or more alcohol dehydrogenases (e.g., adh2, yqhD).

In some embodiments, the at least one branched chain ketoacid decarboxylase enzyme is coexpressed with one or more BCAA transporter(s), for example, a high affinity leucine transporter, e.g., LivKHMGF or low affinity BCAA transporter BrnQ. In some embodiments, the at least one branched chain ketoacid decarboxylase enzyme is coexpressed with one or more BCAA binding protein(s), for example, LivJ. In some embodiments, the engineered bacteria comprise gene sequence(s) encoding at least one branched chain ketoacid decarboxylase enzyme and gene sequence(s) encoding one or more BCAA transporter(s) (e.g., LivKHMGF, brnQ). In some embodiments, the engineered bacteria comprise gene sequence(s) encoding at least one branched chain ketoacid decarboxylase enzyme and gene sequence(s) encoding one or more BCAA binding protein(s) (e.g., livJ). In some embodiments, the engineered bacteria comprise gene sequence(s) encoding at least one branched chain ketoacid decarboxylase enzyme, gene sequence(s) encoding one or more BCAA transporter(s) (e.g., LivKHMGF, brnQ), and gene sequence(s) encoding one or more BCAA binding protein(s) (e.g., livJ). In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one branched chain ketoacid decarboxylase enzyme and genetic modification that reduces export of a branched chain amino acid, e.g., a genetic mutation in a leuE gene or promoter thereof. In one embodiment, the engineered bacteria comprise gene sequence(s) encoding at least one branched chain ketoacid decarboxylase enzyme and a genetic modification that reduces or eliminates branched chain amino acid synthesis, e.g., a genetic mutation in a ilvC gene or promoter thereof.

In some embodiments, the gene sequence(s) encoding the one or more branched chain ketoacid decarboxylase enzyme(s) is expressed under the control of a constitutive promoter. In some embodiments, the gene sequence(s) encoding the one or more branched chain ketoacid decarboxylase enzyme(s) is expressed under the control of an inducible promoter. In some embodiments, the gene sequence(s) encoding the one or more branched chain ketoacid decarboxylase enzyme(s) is expressed under the control of a promoter that is directly or indirectly induced by exogenous environmental conditions. In some embodiments, the gene sequence(s) encoding the one or more branched chain ketoacid decarboxylase enzyme(s) is expressed under the control of a promoter that is directly or indirectly induced by low-oxygen or anaerobic conditions, wherein expression of the gene encoding the branched chain ketoacid decarboxylase enzyme is activated under low-oxygen or anaerobic environments, such as the environment of the mammalian gut. In some embodiments, the gene sequence(s) encoding the one or more branched chain ketoacid decarboxylase enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

B. Branched Chain Keto Acid Dehydrogenase (BCKD)

In one embodiment, the branched chain amino acid catabolism enzyme is a branched chain keto acid dehydrogenase (“BCKD”). Thus, in some embodiments, the engineered bacteria comprise gene sequence(s) encoding one or more copies of a branched chain keto acid dehydrogenase. In some embodiments, the engineered bacteria comprise gene sequence(s) encoding one, two, three, four, five, six, or more copies of a branched chain keto acid dehydrogenase. The one or more copies of branched chain keto acid dehydrogenase can be one or more copies of the same gene or can be different genes encoding branched chain keto acid dehydrogenase, e.g., gene(s) from a different species or otherwise having a different gene sequence. The one or more copies of branched chain keto acid dehydrogenase can be present in the bacterial chromosome or can be present in one or more plasmids. As used herein “branched chain keto acid dehydrogenase” or “BCKD” refers to any polypeptide having enzymatic activity that oxidatively decarboxylates a branched chain keto acid into its respective acyl-CoA derivative. Multiple distinct branched chain keto acid dehydrogenases are known in the art and are available from many microorganism sources, including those disclosed herein, as well as eukaryotic sources, including mammalian sources, e.g. human. In bacteria, branched chain keto acid dehydrogenases are enzyme complexes that oxidatively decarboxylate all three branched chain keto acids (α-ketoisocaproate, α-keto-β-methylvalerate, and α-ketoisovalerate) into their respective acyl-CoA derivatives, (isovaleryl-CoA, α-methylbutyryl-CoA, isobutyryl-CoA). See, for example, Massey et al., Bacteriol Rev., 40(1):42-54, 1976. Moreover, in mammals, dehydrogenases specific for 2-ketoisovalerate (EC 1.2.4.4) and 2-keto-3-methylvalerate and 2-keto-isocaproate (EC 1.2.4.3) have been identified (see, for example, Massey et al., Bacteriol Rev., 40(1):42-54, 1976). In one embodiment, the branched chain amino acid catabolism enzyme is a leucine catabolism enzyme.

In some embodiments, the branched chain keto acid dehydrogenase is encoded by at least one gene encoding a branched chain keto acid dehydrogenase derived from a bacterial species. In some embodiments, the branched chain keto acid dehydrogenase is encoded by at least one gene encoding a branched chain keto acid dehydrogenase derived from a non-bacterial species. In some embodiments, the branched chain keto acid dehydrogenase is encoded by at least one gene derived from a eukaryotic species, e.g., a yeast species or a plant species. In another embodiment, the branched chain keto acid dehydrogenase is encoded by at least one gene derived from a mammalian species, e.g., human

In one embodiment, the at least one gene encoding the branched chain keto acid dehydrogenase is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Proteus, Pseudomonas, Psychrobacter, Ralstonia, Saccharomyces, Salmonella, Sarcina, Serratia, Staphylococcus, Streptococcus, and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Enterococcus faecalis, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Pseudomonas putida, Pseudomonas aeruginosa, Psychrobacter anticus, Proteus vulgaris, Proteus mirabilis, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Streptococcus faecalis, Yersinia enterocolitica, Yersinia mollaretii, Yersinia kristensenii, Yersinia rohdei, and Yersinia aldovae. In some embodiments, the BCKD is encoded by at least one gene derived from Pseudomonas putida. In another embodiment, the BCKD is encoded by at least one gene derived from Pseudomonas aeruginosa. In another embodiment, the BCKD is encoded by at least one gene derived from Streptococcus faecalis. In another embodiment, the BCKD is encoded by at least one gene derived from Proteus vulgaris. In another embodiment, the BCKD is encoded by at least one gene derived from Bacillus subtilis. In another embodiment, the BCKD is encoded by at least one gene derived from Streptococcus faecalis. In another embodiment, the BCKD is encoded by at least one gene derived from Bacillus subtilis.

In some embodiments, the at least one gene encoding the branched chain keto acid dehydrogenase has been codon-optimized for use in the recombinant bacterial cell. In one embodiment, the at least one gene encoding the branched chain keto acid dehydrogenase has been codon-optimized for use in Escherichia coli. In one embodiment, the at least one gene encoding the branched chain keto acid dehydrogenase is a branched chain keto acid dehydrogenase gene from Pseudomonas aeruginosa PAO1. In one embodiment, the at least one gene encoding the branched chain keto acid dehydrogenase comprises the bkdA1-bkdA2-bkdB-lpdV operon. In one embodiment, the bkdA1-bkdA2-bkdB-lpdV operon is at least 90% identical to the uppercase sequence set forth in SEQ ID NO:3. In another embodiment, the bkdA1-bkdA2-bkdB-lpdV operon comprises the uppercase sequence set forth in SEQ ID NO:3. In another embodiment, the at least one gene encoding the branched chain keto acid dehydrogenase comprises the LeuDH-bkdA1-bkdA2-bkdB-lpdV operon. In one embodiment, the LeuDH-bkdA1-bkdA2-bkdB-lpdV operon is at least 90% identical to the uppercase sequence set forth in SEQ ID NO:4. In another embodiment, the LeuDH-bkdA1-bkdA2-bkdB-lpdV operon comprises the uppercase sequence as set forth in SEQ ID NO:4. In another embodiment, the at least one gene encoding is Alpha-ketoisovalerate dehydrogenase (EC 1.2.4.4). In another embodiment, the at least one gene encoding the branched chain keto acid dehydrogenase is 2-oxoisocaproate dehydrogenase (EC 1.2.4.3). In yet another embodiment, the at least one gene encoding the branched chain keto acid dehydrogenase is the human dehydrogenase/decarboxylase (E1). In another embodiment, the at least one gene encoding the branched chain keto acid dehydrogenase comprises the human E1α and two E1β subunits. In another embodiment, the at least one gene encoding the branched chain keto acid dehydrogenase comprises the human dihydrolipoyl transacylase (E2) gene. In yet another embodiment, the at least one gene encoding the branched chain keto acid dehydrogenase comprises the human dihydrolipoamide dehydrogenase (E3) gene. In another embodiment, the at least one gene encoding the branched chain keto acid dehydrogenase comprises the human dehydrogenase/decarboxylase (E1) gene, the human dihydrolipoly transacylase (E2) gene, and the human dihydrolipoamide dehydrogenase (E3) gene.

When a branched chain amino acid catabolism enzyme is expressed in the recombinant bacterial cells disclosed herein, the bacterial cells catabolize more branched chain amino acid, e.g., leucine, than unmodified bacteria of the same bacterial subtype under the same conditions (e.g., culture or environmental conditions). Thus, the genetically engineered bacteria comprising at least one heterologous gene encoding a branched chain keto acid dehydrogenase may be used to catabolize excess branched chain amino acids, e.g., leucine, to treat a disease associated with the catabolism of a branched chain amino acid, including Maple Syrup Urine Disease (MSUD). In some embodiments, the branched chain keto acid dehydrogenase is co-expressed with an additional branched chain amino acid dehydrogenase, e.g., a leucine dehydrogenase, e.g., leuDH, described in more detail below.

The present disclosure further comprises genes encoding functional fragments of a branched chain keto acid dehydrogenase or functional variants of branched chain keto acid dehydrogenase. As used herein, the term “functional fragment thereof” or “functional variant thereof” of branched chain keto acid dehydrogenase relates to a sequence having qualitative biological activity in common with the wild-type branched chain keto acid dehydrogenase from which the fragment or variant was derived. For example, a functional fragment or a functional variant of a mutated branched chain keto acid dehydrogenase protein is one which retains essentially the same ability to catabolize leucine or other BCAA as the protein from which the functional fragment or functional variant was derived. For example, a polypeptide having branched chain keto acid dehydrogenase activity may be truncated at the N-terminus or C-terminus and the retention of enzyme activity assessed using assays known to those of skill in the art, including the exemplary assays provided herein. In one embodiment, the recombinant bacterial cell disclosed herein comprises a heterologous gene encoding a branched chain keto acid dehydrogenase functional variant. In another embodiment, the recombinant bacterial cell disclosed herein comprises a heterologous gene encoding a branched chain keto acid dehydrogenase functional fragment.

Assays for testing the activity of a branched chain keto acid dehydrogenase, a branched chain keto acid dehydrogenase functional variant, or a branched chain keto acid dehydrogenase functional fragment are well known to one of ordinary skill in the art. For example, branched chain keto acid dehydrogenase activity can be assessed by expressing the protein, functional variant, or fragment thereof, in a recombinant bacterial cell that lacks endogenous branched chain keto acid dehydrogenase activity. Also, activity can be assessed using the enzymatic assay methods as described by Sykes et al. (J. Bacteriol., 169(4):1619-1625, 1987), Sokatch et al. (J. Bacteriol., 148:639-646, 1981), and Massey et al. (Bacteriol. Rev., 40(1):42-54, 1976).

The present disclosure encompasses genes encoding a branched chain keto acid dehydrogenase comprising amino acids in its sequence that are substantially the same as an amino acid sequence described herein. In some embodiments, the at least one gene encoding a branched chain keto acid dehydrogenase is mutagenized, mutants exhibiting increased activity are selected, and the mutagenized gene(s) encoding the branched chain keto acid dehydrogenase are isolated and inserted into the bacterial cell. In some embodiments, the at least one gene encoding a branched chain keto acid dehydrogenase is mutagenized, mutants exhibiting decreased activity are selected, and the mutagenized gene(s) encoding the branched chain keto acid dehydrogenase are isolated and inserted into the bacterial cell. The gene comprising the modifications described herein may be present on a plasmid or chromosome.

In one embodiment, the at least one gene encoding the branched chain keto acid dehydrogenase comprises the bkdA1-bkdA2-bkdB-lpdV operon. In one embodiment, the at least one BCKD gene has at least about 80% identity with the entire uppercase sequence of SEQ ID NO:3. Accordingly, in one embodiment, the at least one BCKD gene has at least about 90% identity with the entire uppercase sequence of SEQ ID NO:3. Accordingly, in one embodiment, the at least one BCKD gene has at least about 95% identity with the entire uppercase sequence of SEQ ID NO:3. Accordingly, in one embodiment, the at least one BCKD gene has at least about 85%, 86%, 87%, 88%, 89%, 90%, 91%, 92%, 93%, 94%, 95%, 96%, 97%, 98%, or 99% identity with the entire uppercase sequence of SEQ ID NO:3. In another embodiment, the at least one BCKD gene comprises the uppercase sequence of SEQ ID NO:3. In yet another embodiment the at least one BCKD gene consists of the uppercase sequence of SEQ ID NO:3.

In another embodiment, the at least one BCKD gene is coexpressed with an additional branched chain amino acid dehydrogenase. In one embodiment, the at least one BCKD gene is coexpressed with a leucine dehydrogenase, e.g., leuDH. In another embodiment, the at least one gene encoding the branched chain keto acid dehydrogenase comprises the leuDH-bkdA1-bkdA2-bkdB-lpdV operon. In one embodiment, the at least one BCKD gene has at least about 80% identity with the entire uppercase sequence of SEQ ID NO:4. Accordingly, in one embodiment, the at least one BCKD gene has at least about 90% identity with the entire uppercase sequence of SEQ ID NO:4. Accordingly, in one embodiment, the at least one BCKD gene has at least about 95% identity with the entire uppercase sequence of SEQ ID NO:4. Accordingly, in one embodiment, the at least one BCKD gene has at least about 85%, 86%, 87%, 88%, 89%, 90%, 91%, 92%, 93%, 94%, 95%, 96%, 97%, 98%, or 99% identity with the entire uppercase sequence of SEQ ID NO:4. In another embodiment, the at least one BCKD gene comprises the uppercase sequence of SEQ ID NO:4. In yet another embodiment the at least one BCKD gene consists of the uppercase sequence of SEQ ID NO:4. In another embodiment, the at least one BCKD gene is coexpressed with a branched chain amino acid aminotransferase, e.g., ilvE, described in more detail below.

In other embodiments, the engineered bacteria comprise gene sequence(s) encoding at least one branched chain keto acid dehydrogenase enzyme and further comprise gene sequence encoding one or more polypeptides selected from other branched chain amino acid catabolism enzyme(s), BCAA transporter(s), and BCAA binding protein(s). Thus, in some embodiments, the at least one branched chain keto acid dehydrogenase enzyme is coexpressed with an additional branched chain amino acid catabolism enzyme, e.g., a branched chain amino acid dehydrogenase, amino acid oxidase (also known as amino acid deaminase), and/or aminotransferase. In some embodiments, the at least one branched chain keto acid dehydrogenase gene is coexpressed with a leucine dehydrogenase, e.g., (leuDH), described in more detail below. In other embodiments, the at least one branched chain keto acid dehydrogenase gene is coexpressed with a branched chain amino acid aminotransferase, e.g., ilvE, described in more detail below. In other embodiments, the at least one branched chain keto acid dehydrogenase is coexpressed with an amino acid deaminase, e.g., L-AAD, described in more detail below. In some embodiments, the engineered bacteria comprise gene sequence(s) encoding at least one branched chain keto acid dehydrogenase enzyme and gene sequence(s) encoding one or more branched chain amino acid dehydrogenase(s) (e.g., leuDH). In some embodiments, the engineered bacteria comprise gene sequence(s) encoding at least one branched chain keto acid dehydrogenase enzyme and gene sequence(s) encoding one or more amino acid oxidase(s) (e.g. L-AAD)). In some embodiments, the engineered bacteria comprise gene sequence(s) encoding at least one branched chain keto acid dehydrogenase enzyme and gene sequence(s) encoding one or more aminotransferase(s) (e.g., ilvE).

In some embodiments, the at least one branched chain keto acid dehydrogenase enzyme is coexpressed with one or more BCAA transporter(s), for example, a high affinity leucine transporter, e.g., LivKHMGF or low affinity BCAA transporter BrnQ. In some embodiments, the at least one branched chain keto acid dehydrogenase enzyme is coexpressed with one or more BCAA binding protein(s), for example, LivJ. In some embodiments, the engineered bacteria comprise gene sequence(s) encoding at least one branched chain keto acid dehydrogenase enzyme and gene sequence(s) encoding one or more BCAA transporter(s) (e.g., LivKHMGF, brnQ). In some embodiments, the engineered bacteria comprise gene sequence(s) encoding at least one branched chain keto acid dehydrogenase enzyme and gene sequence(s) encoding one or more BCAA binding protein(s) (e.g., livJ). In some embodiments, the engineered bacteria comprise gene sequence(s) encoding at least one branched chain keto acid dehydrogenase enzyme, gene sequence(s) encoding one or more BCAA transporter(s) (e.g., LivKHMGF, brnQ), and gene sequence(s) encoding one or more BCAA binding protein(s) (e.g., livJ). In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one branched chain keto acid dehydrogenase enzyme and genetic modification that reduces export of a branched chain amino acid, e.g., a genetic mutation in a leuE gene or promoter thereof. In one embodiment, the engineered bacteria comprise gene sequence(s) encoding at least one branched chain keto acid dehydrogenase enzyme and a genetic modification that reduces or eliminates branched chain amino acid synthesis, e.g., a genetic mutation in a ilvC gene or promoter thereof.

In some embodiments, the gene sequence(s) encoding the one or more branched chain keto acid dehydrogenase enzyme(s) is expressed under the control of a constitutive promoter. In some embodiments, the gene sequence(s) encoding the one or more branched chain keto acid dehydrogenase enzyme(s) is expressed under the control of an inducible promoter. In some embodiments, the gene sequence(s) encoding the one or more branched chain keto acid dehydrogenase enzyme(s) is expressed under the control of a promoter that is directly or indirectly induced by exogenous environmental conditions. In some embodiments, the gene sequence(s) encoding the one or more branched chain keto acid dehydrogenase enzyme(s) is expressed under the control of a promoter that is directly or indirectly induced by low-oxygen or anaerobic conditions, wherein expression of the gene encoding the branched chain amino acid catabolism enzyme is activated under low-oxygen or anaerobic environments, such as the environment of the mammalian gut. In some embodiments, the gene sequence(s) encoding the one or more branched chain keto acid dehydrogenase enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

C. Branched Chain Amino Acid Deamination Enzymes

In one embodiment, the branched chain amino acid catabolism enzyme is a branched chain amino acid deamination enzyme. Thus, in some embodiments, the engineered bacteria comprise gene sequence(s) encoding one or more copies of a branched chain amino acid deamination enzyme. In some embodiments, the engineered bacteria comprise gene sequence(s) encoding one, two, three, four, five, six, or more copies of a branched chain amino acid deamination enzyme. The one or more copies of branched chain amino acid deamination enzyme can be one or more copies of the same gene or can be different genes encoding branched chain amino acid deamination enzyme, e.g., gene(s) from a different species or otherwise having a different gene sequence. The one or more copies of branched chain amino acid deamination enzyme can be present in the bacterial chromosome or can be present in one or more plasmids. As used herein, the term “branched chain amino acid deamination enzyme” refers to an enzyme involved in the deamination, or the removal of an amine group, of a branched chain amino acid, which produces a corresponding branched chain alpha-keto acid (e.g., α-ketoisocaproate, α-keto-βmethylvalerate, α-ketoisovalerate). Enzymes involved in the deamination of branched chain amino acids are well known to those of skill in the art. For example, in bacteria, leucine dehydrogenase (LeuDH, leuDH), e.g., derived from Pseudomonas aeruginosa PA01, is capable of catalyzing the reversible deamination of branched chain amino acids, such as leucine, into their corresponding keto-acid counterpart (Baker et al., Structure, 3(7):693-705, 1995). Similarly, the ilvE gene from E. coli Nissle has also been shown to catalyze the reversible deamination of branched chain amino acids (Peng et al., J. Bact., 139(2):339-45, 1979; Kline et al., J. Bact., 130(2):951-3, 1977). The L-AAD gene, e.g., derived from Proteus vulgaris or Proteus mirabilis, has also been shown to catalyze the irreversible deamination of branched chain amino acids (Song et al., Scientific Reports, Nature, 5:12694; DOI: 10:1038/srep12694 (2015)).

In one embodiment, the branched chain amino acid deamination enzyme increases the rate of branched chain amino acid deamination in the cell. In one embodiment, the branched chain amino acid deamination enzyme decreases the level of branched chain amino acid in the cell as compared to the level of its corresponding alpha-keto acid in the cell. In another embodiment, the branched chain amino acid deamination enzyme increases the level of alpha-keto acid in the cell as compared to the level of its corresponding branched chain amino acid in the cell.

In one embodiment, the branched chain amino acid deamination enzyme is a leucine deamination enzyme. In another embodiment, the branched chain amino acid deamination enzyme is an isoleucine deamination enzyme. In another embodiment, the branched chain amino acid deamination enzyme is a valine deamination enzyme. In another embodiment, the branched chain amino acid deamination enzyme is involved in the deamination of leucine, isoleucine, and valine. In another embodiment, the branched chain amino acid deamination enzyme is involved in the deamination of leucine and valine, isoleucine and valine, or leucine and isoleucine. In some embodiments, the branched chain amino acid deamination enzyme is encoded by a branched chain amino acid deamination enzyme gene derived from a bacterial species. In some embodiments, the branched chain amino acid deamination enzyme is encoded by a branched chain amino acid deamination enzyme gene derived from a non-bacterial species. In some embodiments, the branched chain amino acid deamination enzyme is encoded by a branched chain amino acid deamination enzyme gene derived from a eukaryotic species, e.g., a yeast species or a plant species. In another embodiment, the branched chain amino acid deamination enzyme is encoded by a branched chain amino acid deamination enzyme gene derived from a mammalian species, e.g., human

In other embodiments, the engineered bacteria comprise gene sequence(s) encoding at least one branched chain amino acid deamination enzyme and further comprise gene sequence encoding one or more polypeptides selected from other branched chain amino acid catabolism enzyme(s), BCAA transporter(s), and BCAA binding protein(s). Thus, in some embodiments, the at least one branched chain amino acid deamination enzyme is coexpressed with another branched chain amino acid deamination enzyme. In some embodiments, the at least one branched chain amino acid deamination enzyme is coexpressed with another branched chain amino acid catabolism enzyme, e.g., a ketoacid decarboxylase, such as KivD. In some embodiments, the at least one branched chain amino acid deamination enzyme is coexpressed with an aldehyde dehydrogenase, e.g., PadA, described in more detail below. In some embodiments, the at least one branched chain amino acid deamination enzyme is coexpressed with an alcohol dehydrogenase, e.g., Adh2, YqhD, described in more detail below. In some embodiments, the at least one branched chain amino acid deamination enzyme is coexpressed with one or more BCAA transporter(s), for example, a high affinity leucine transporter, e.g., LivKHMGF (SEQ ID NO: 91) or low affinity BCAA transporter BrnQ (SEQ ID NO: 64). In some embodiments, the at least one branched chain amino acid deamination enzyme is coexpressed with one or more BCAA binding protein(s), for example, LivJ (SEQ ID NO: 12). In some embodiments, the engineered bacteria comprise gene sequence(s) encoding at least one branched chain amino acid deamination enzyme and genetic modification that reduces export of a branched chain amino acid, e.g., a genetic mutation in a leuE gene or promoter thereof. In one embodiment, the engineered bacteria comprise gene sequence(s) encoding at least one branched chain amino acid deamination enzyme and a genetic modification that reduces or eliminates branched chain amino acid synthesis, e.g., a genetic mutation in a ilvC gene or promoter thereof.

In some embodiments, the gene sequence(s) encoding the one or more branched chain amino acid deamination enzyme(s) is expressed under the control of a constitutive promoter. In some embodiments, the gene sequence(s) encoding the one or more branched chain amino acid deamination enzyme(s) is expressed under the control of an inducible promoter. In some embodiments, the gene sequence(s) encoding the one or more branched chain amino acid deamination enzyme(s) is expressed under the control of a promoter that is directly or indirectly induced by exogenous environmental conditions. In some embodiments, the gene sequence(s) encoding the one or more branched chain amino acid deamination enzyme(s) is expressed under the control of a promoter that is directly or indirectly induced by low-oxygen or anaerobic conditions, wherein expression of the gene encoding the branched chain amino acid deamination enzyme is activated under low-oxygen or anaerobic environments, such as the environment of the mammalian gut. In some embodiments, the gene sequence(s) encoding the one or more branched chain amino acid deamination enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

Non-limiting examples of branched chain amino acid deamination enzymes include leucine dehydrogenase, L-amino acid deaminase, and branched chain amino acid aminotransferase, and are described in more detail in the subsections, below.

(1) Branched Chain Amino Acid Dehydrogenases (Leucine Dehydrogenase)

In some embodiment, the branched chain amino acid deamination enzyme is a branch chain amino acid dehydrogenase. Thus, in some embodiments, the engineered bacteria comprise gene sequence(s) encoding one or more copies of a branch chain amino acid dehydrogenase. In some embodiments, the engineered bacteria comprise gene sequence(s) encoding one, two, three, four, five, six, or more copies of a branch chain amino acid dehydrogenase. The one or more copies of branch chain amino acid dehydrogenase can be one or more copies of the same gene or can be different genes encoding branch chain amino acid dehydrogenase, e.g., gene(s) from a different species or otherwise having a different gene sequence. The one or more copies of branch chain amino acid dehydrogenase can be present in the bacterial chromosome or can be present in one or more plasmids.

In some embodiments, the branched chain amino acid deamination enzyme is leucine dehydrogenase (“leuDH” or “leuDH”). As used herein “leucine dehydrogenase” refers to any polypeptide having enzymatic activity that deaminates leucine to its corresponding ketoacid, alpha-ketoisocaproate (KIC), deaminates valine to its corresponding ketoacid, ketoisovalerate (MV), and deaminates isoleucine to its corresponding ketoacid, ketomethylvalerate (KMV). In some embodiments, the bacterial cells disclosed herein comprise a heterologous gene encoding a leucine dehydrogenase enzyme and are capable of converting leucine, valine, and/or isoleucine to their respective α-keto acids. For example, the leucine dehydrogenase enzyme LeuDH is capable of metabolizing leucine and a cytosolically active LeuDH should generally exhibit the ability to convert valine, isoleucine, and leucine to ketoisovalerate, ketomethylvalerate, and ketoisocaproate, respectively. Leucine dehydrogenase employs the co-factor NAD+. In some embodiments, leuDH encodes an octamer.

Multiple distinct leucine dehydrogenases (EC 1.4.1.9) are known in the art and are available from many microorganism sources, including those disclosed herein, as well as from eukaryotic sources (see, for example, Baker et al., Structure, 3(7):693-705, 1995). In some embodiments, the branched chain amino acid deamination enzyme is encoded by at least one gene encoding a branched chain amino acid deamination enzyme derived from a bacterial species. In some embodiments, the branched chain amino acid deamination enzyme is encoded by at least one gene encoding a branched chain amino acid deamination enzyme derived from a non-bacterial species. In some embodiments, the branched chain amino acid deamination enzyme is encoded by at least one gene derived from a eukaryotic species, e.g., a yeast species or a plant species. In another embodiment, the branched chain amino acid deamination enzyme is encoded by at least one gene derived from a mammalian species, e.g., human

In one embodiment, the engineered bacteria comprise gene sequence(s) encoding one or more branch chain amino acid dehydrogenase(s), e.g., leucine dehydrogenase enzyme(s). In some embodiments, the branch chain amino acid dehydrogenase, e.g., leucine dehydrogenase enzyme is derived from an organism of the genus or species that includes, but is not limited to, Bacillus, Brevibacillus, Geobacillus, Lysinibacillus, Moorella, Natrialba, Pseudomonas, Sporosarcinia, and Thermoactinomyces. In some embodiments, the leuDH gene is encoded by a gene derived from Bacillus caldolyticus, Bacillus cereus, Bacillus licheniformis, Bacillus megaterium, Bacillus mycoides, Bacillus niger, Bacillus pumilus, Bacillus subtilis, Brevibacillus brevis, Geobacillus stearothermophilus, Lysinibacillus sphaeriscus, Moorella Thermoacetica, Natrialba magadii, Sporosarcina psychorophila, Thermoactinomyces intermedius, Pseudomonas aeruginosa, or Pseudomonas resinovorans. In some embodiments, the branch chain amino acid dehydrogenase enzyme, e.g., leucine dehydrogenase, is encoded by at least one gene derived from Pseudomonas aeruginosa PA01. In some embodiments, the branch chain amino acid dehydrogenase enzyme, e.g., leucine dehydrogenase, is encoded by at least one gene derived from Bacillus cereus. In some embodiments, the at least one gene encoding the branch chain amino acid dehydrogenase enzyme, e.g., leucine dehydrogenase, has been codon-optimized for use in the recombinant bacterial cell. In one embodiment, the at least one gene encoding the branch chain amino acid dehydrogenase enzyme, e.g., leucine dehydrogenase, has been codon-optimized for use in Escherichia coli. For example, a codon-optimized LeuDH sequence is set forth as SEQ ID NO: 20 and 58.

When a branch chain amino acid dehydrogenase enzyme, e.g., leucine dehydrogenase, is expressed in the recombinant bacterial cells disclosed herein, the bacterial cells catabolize more branched chain amino acid, e.g., leucine, isoleucine, and valine, than unmodified bacteria of the same bacterial subtype under the same conditions (e.g., culture or environmental conditions). Thus, the genetically engineered bacteria comprising at least one heterologous gene encoding a branch chain amino acid dehydrogenase enzyme, e.g., leucine dehydrogenase, may be used to catabolize excess branched chain amino acids, e.g., leucine, valine, and isoleucine, to treat a disease associated with the deamination of a branched chain amino acid, including Maple Syrup Urine Disease (MSUD) as well as other disease provided herein.

The present disclosure further comprises genes encoding functional fragments of a branch chain amino acid dehydrogenase enzyme, e.g., leucine dehydrogenase, or functional variants of branch chain amino acid dehydrogenase enzyme, e.g., leucine dehydrogenase. The present disclosure encompasses genes encoding a branch chain amino acid dehydrogenase enzyme, e.g., leucine dehydrogenase, comprising amino acids in its sequence that are substantially the same as an amino acid sequence described herein. As used herein, the term “functional fragment thereof” or “functional variant thereof” of a branch chain amino acid dehydrogenase enzyme, e.g., a leucine dehydrogenase, gene relates to a sequence having qualitative biological activity in common with the wild-type branch chain amino acid dehydrogenase, e.g., a leucine dehydrogenase, from which the fragment or variant was derived. For example, a functional fragment or a functional variant of a mutated branch chain amino acid dehydrogenase protein, e.g., a leucine dehydrogenase, is one which retains essentially the same ability to catabolize BCAAs as the branch chain amino acid dehydrogenase protein, e.g., a leucine dehydrogenase, from which the functional fragment or functional variant was derived. For example, a polypeptide having branch chain amino acid dehydrogenase enzyme, e.g., a leucine dehydrogenase, activity may be truncated at the N-terminus or C-terminus and the retention of branch chain amino acid dehydrogenase enzyme, e.g., a leucine dehydrogenase, activity assessed using assays known to those of skill in the art, including the exemplary assays provided herein. In one embodiment, the recombinant bacterial cell disclosed herein comprises a heterologous gene encoding an a branch chain amino acid dehydrogenase enzyme, e.g., a leucine dehydrogenase, functional variant. In another embodiment, the recombinant bacterial cell disclosed herein comprises a heterologous gene encoding a branch chain amino acid dehydrogenase enzyme, e.g., a leucine dehydrogenase, functional fragment.

Assays for testing the activity of a branch chain amino acid dehydrogenase enzyme functional variant or functional fragment, e.g., a leucine dehydrogenase functional variant or a leucine dehydrogenase functional fragment are well known to one of ordinary skill in the art. For example, leucine dehydrogenase activity can be assessed by expressing the protein, functional variant, or fragment thereof, in a recombinant bacterial cell that lacks endogenous leucine dehydrogenase activity. Also, activity can be assessed using the enzymatic assay methods as described by Soda et al. (Biochem. Biophys. Res. Commun., 44:931, 1971), and Ohshima et al. (J. Biol. Chem., 253:5719, 1978), the entire contents of each of which are expressly incorporated herein by reference.

In some embodiments, the at least one gene encoding a branch chain amino acid dehydrogenase enzyme, e.g., leucine dehydrogenase is mutagenized, mutants exhibiting increased activity are selected, and the mutagenized gene(s) encoding the branch chain amino acid dehydrogenase enzyme, e.g., leucine dehydrogenase, are isolated and inserted into the bacterial cell. In some embodiments, the at least one gene encoding a branch chain amino acid dehydrogenase enzyme, e.g., leucine dehydrogenase is mutagenized, mutants exhibiting decreased activity are selected, and the mutagenized gene(s) encoding the branch chain amino acid dehydrogenase enzyme, e.g., leucine dehydrogenase, are isolated and inserted into the bacterial cell. The gene comprising the modifications described herein may be present on a plasmid or chromosome.

In some embodiments, the engineered bacteria comprise gene sequence(s) encoding at least one branch chain amino acid dehydrogenase enzyme, e.g., leucine dehydrogenase, and further comprise gene sequence encoding one or more polypeptides selected from other branched chain amino acid catabolism enzyme(s), BCAA transporter(s), and BCAA binding protein(s). Thus, in some embodiments, the at least one branch chain amino acid dehydrogenase enzyme, e.g., leucine dehydrogenase, is coexpressed with an additional branched chain amino acid deamination enzyme, e.g., branched chain amino acid dehydrogenase, a branched chain aminotransferase, and/or amino acid oxidase (also known as amino acid deaminase). For example, in some embodiments, the branch chain amino acid dehydrogenase enzyme, e.g., leucine dehydrogenase, is coexpressed with one or more other branch chain amino acid dehydrogenase enzyme(s). In some embodiments, the branch chain amino acid dehydrogenase enzyme, e.g., leucine dehydrogenase, is coexpressed with one or more branched chain aminotransferase enzyme(s), for example, ilvE. In some embodiments, the branch chain amino acid dehydrogenase enzyme, e.g., leucine dehydrogenase, is coexpressed with one or more amino acid oxidase enzyme(s), e.g., L-AAD. In some embodiments, the engineered bacteria comprise gene sequence(s) encoding at least branch chain amino acid dehydrogenase enzyme, e.g., leucine dehydrogenase enzyme, and gene sequence(s) encoding one or more other branched chain amino acid dehydrogenase(s). In some embodiments, the engineered bacteria comprise gene sequence(s) encoding at least one branch chain amino acid dehydrogenase enzyme, e.g., leucine dehydrogenase enzyme, and gene sequence(s) encoding one or more amino acid oxidase(s) (e.g. L-AAD)). In some embodiments, the engineered bacteria comprise gene sequence(s) encoding at least one branch chain amino acid dehydrogenase enzyme, e.g., leucine dehydrogenase enzyme, and gene sequence(s) encoding one or more BCAA aminotransferase(s) (e.g., ilvE).

In some embodiments, the branch chain amino acid dehydrogenase enzyme, e.g., leucine dehydrogenase, is coexpressed with one or more other branched chain amino acid catabolism enzyme(s), for example, a ketoacid decarboxylase, such as kivD. In some embodiments, the branch chain amino acid dehydrogenase enzyme, e.g., leucine dehydrogenase, is coexpressed with one or more other branched chain amino acid catabolism enzyme(s), for example, a branched chain alcohol dehydrogenase, such as adh2 or yqhD and/or a branched chain aldehyde dehydrogenase, such as padA. In other embodiments, the branch chain amino acid dehydrogenase enzyme, e.g., leucine dehydrogenase, is coexpressed with a second branched chain amino acid catabolism enzyme, for example, kivD, and a branched chain alcohol dehydrogenase, for example, adh2 or YqhD, and/or a branched chain aldehyde dehydrogenase, for example, padA, each of which are described in more detail herein. In some embodiments, the engineered bacteria comprise gene sequence(s) encoding at least one branch chain amino acid dehydrogenase enzyme, e.g., leucine dehydrogenase enzyme, and gene sequence(s) encoding one or more keto-acid decarboxylase(s) (e.g., kivD). In some embodiments, the engineered bacteria comprise gene sequence(s) encoding at least one branch chain amino acid dehydrogenase enzyme, e.g., leucine dehydrogenase enzyme, and gene sequence(s) encoding one or more aldehyde dehydrogenase(s) (e.g., padA). In some embodiments, the engineered bacteria comprise gene sequence(s) encoding at least one branch chain amino acid dehydrogenase enzyme, e.g., leucine dehydrogenase enzyme, and gene sequence(s) encoding one or more alcohol dehydrogenase(s) (e.g., adh2, yqhD). In some embodiments, the engineered bacteria comprise gene sequence(s) encoding at least one branch chain amino acid dehydrogenase enzyme, e.g., leucine dehydrogenase enzyme, gene sequence(s) encoding one or more aldehyde dehydrogenase(s) (e.g., padA), and gene sequence(s) encoding one or more alcohol dehydrogenase(s) (e.g., adh2, yqhD). In some embodiments, the engineered bacteria comprise gene sequence(s) encoding at least one branch chain amino acid dehydrogenase enzyme, e.g., leucine dehydrogenase enzyme, gene sequence(s) encoding one or more keto-acid decarboxylase(s) (e.g., kivD), and gene sequence(s) encoding one or more aldehyde dehydrogenase(s) (e.g., padA). In some embodiments, the engineered bacteria comprise gene sequence(s) encoding at least one branch chain amino acid dehydrogenase enzyme, e.g., leucine dehydrogenase enzyme, gene sequence(s) encoding one or more keto-acid decarboxylase(s) (e.g., kivD), and gene sequence(s) encoding one or more alcohol dehydrogenase(s) (e.g., adh2, yqhD). In some embodiments, the engineered bacteria comprise gene sequence(s) encoding at least branch chain amino acid dehydrogenase enzyme, e.g., leucine dehydrogenase enzyme, gene sequence(s) encoding one or more keto-acid decarboxylase(s) (e.g., kivD), gene sequence(s) encoding one or more aldehyde dehydrogenase(s) (e.g., padA), and gene sequence(s) encoding one or more alcohol dehydrogenase(s) (e.g., adh2, yqhD).

In some embodiments, the at least one branch chain amino acid dehydrogenase enzyme, e.g., leucine dehydrogenase enzyme is coexpressed with one or more BCAA transporter(s), for example, a high affinity leucine transporter, e.g., LivKHMGF and/or low affinity BCAA transporter BrnQ. In some embodiments, the at least one branch chain amino acid dehydrogenase enzyme, e.g., leucine dehydrogenase enzyme is coexpressed with one or more BCAA binding protein(s), for example, LivJ. In some embodiments, the engineered bacteria comprise gene sequence(s) encoding at least one branch chain amino acid dehydrogenase enzyme, e.g., leucine dehydrogenase enzyme and gene sequence(s) encoding one or more BCAA transporter(s) (e.g., LivKHMGF, brnQ). In some embodiments, the engineered bacteria comprise gene sequence(s) encoding at least one branch chain amino acid dehydrogenase enzyme, e.g., leucine dehydrogenase enzyme and gene sequence(s) encoding one or more BCAA binding protein(s) (e.g., livJ). In some embodiments, the engineered bacteria comprise gene sequence(s) encoding at least one branch chain amino acid dehydrogenase enzyme, e.g., leucine dehydrogenase enzyme, gene sequence(s) encoding one or more BCAA transporter(s) (e.g., LivKHMGF, brnQ), and gene sequence(s) encoding one or more BCAA binding protein(s) (e.g., livJ). In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one branch chain amino acid dehydrogenase enzyme, e.g., leucine dehydrogenase enzyme, and genetic modification that reduces export of a branched chain amino acid, e.g., a genetic mutation in a leuE gene or promoter thereof. In one embodiment, the engineered bacteria comprise gene sequence(s) encoding at least one branch chain amino acid dehydrogenase enzyme, e.g., leucine dehydrogenase enzyme, and a genetic modification that reduces or eliminates branched chain amino acid synthesis, e.g., a genetic mutation in a ilvC gene or promoter thereof.

In some embodiments, the gene sequence(s) encoding the one or more branch chain amino acid dehydrogenase enzyme(s), e.g., leucine dehydrogenase enzyme(s) is expressed under the control of a constitutive promoter. In some embodiments, the gene sequence(s) encoding the one or more branch chain amino acid dehydrogenase enzyme(s), e.g., leucine dehydrogenase enzyme(s) is expressed under the control of an inducible promoter. In some embodiments, the gene sequence(s) encoding the one or more branch chain amino acid dehydrogenase enzyme(s), e.g., leucine dehydrogenase enzyme(s) is expressed under the control of a promoter that is directly or indirectly induced by exogenous environmental conditions. In some embodiments, the gene sequence(s) encoding the one or more branch chain amino acid dehydrogenase enzyme(s), e.g., leucine dehydrogenase enzyme(s) is expressed under the control of a promoter that is directly or indirectly induced by low-oxygen or anaerobic conditions, wherein expression of the gene encoding the branch chain amino acid dehydrogenase enzyme, e.g., leucine dehydrogenase enzyme is activated under low-oxygen or anaerobic environments, such as the environment of the mammalian gut. In some embodiments, the gene sequence(s) encoding the one or more branch chain amino acid dehydrogenase enzyme(s), e.g., leucine dehydrogenase enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

In some embodiments, the branched chain amino acid dehydrogenase is leucine dehydrogenase. Thus, income embodiments, the engineered bacteria comprise gene sequence of SEQ ID NO: 20 and/or 58. The present disclosure further comprises genes encoding functional fragments of leucine dehydrogenase, or functional variants of leucine dehydrogenase. The present disclosure encompasses genes encoding leucine dehydrogenase, comprising amino acids in its sequence that are substantially the same as an amino acid sequence described herein. In some embodiments, the at least one leuDH gene has at least about 80% identity with SEQ ID NO:20. Accordingly, in one embodiment, the at least one leuDH gene has at least about 90% identity with SEQ ID NO:20. Accordingly, in one embodiment, the at least one leuDH gene has at least about 95% identity with SEQ ID NO:20. Accordingly, in one embodiment, the at least one leuDH gene has at least about 85%, 86%, 87%, 88%, 89%, 90%, 91%, 92%, 93%, 94%, 95%, 96%, 97%, 98%, or 99% identity with SEQ ID NO:20. In another embodiment, the at least one leuDH gene comprises SEQ ID NO:20. In yet another embodiment the at least one leuDH gene consists of SEQ ID NO:20. In another embodiment, the at least one gene encoding the leucine dehydrogenase belongs to the family oxidoreductases (EC 1.4.1.9). In yet another embodiment, the at least one gene encoding the leucine dehydrogenase is the L-leucine:NAD+oxidoreductase. In one embodiment, the leucine dehydrogenase gene has been codon-optimized for use in the recombinant bacterial cell. In one embodiment, the leucine dehydrogenase gene has been codon-optimized for use in Escherichia coli. For example, a codon-optimized leuDH sequence is set forth as SEQ ID NO:20 and SEQ ID NO: 58.

2) Amino Acid Aminotransferases

In another embodiment, the branched chain amino acid deamination enzyme is a branched chain amino acid aminotransferase. In some embodiments, the engineered bacteria comprise gene sequence(s) encoding one or more copies of a branched chain amino acid aminotransferase. In some embodiments, the engineered bacteria comprise gene sequence(s) encoding one, two, three, four, five, six, or more copies of a branched chain amino acid aminotransferase. The one or more copies of branched chain amino acid aminotransferase can be one or more copies of the same gene or can be different genes encoding branched chain amino acid aminotransferase, e.g., gene(s) from a different species or otherwise having a different gene sequence. The one or more copies of branched chain amino acid aminotransferase can be present in the bacterial chromosome or can be present in one or more plasmids. As used herein “branched chain amino acid aminotransferase” refers to any polypeptide having enzymatic activity that deaminates a branched chain amino acid, e.g., leucine, valine, isoleucine to its corresponding ketoacid, e.g., alpha-ketoisocaproate (KIC) (EC 2.6.1.42), alpha-ketoisovalerate, alpha-keto-beta-methylvalerate. Multiple distinct branched chain amino acid aminotransferases are known in the art and are available from many microorganism sources, including those disclosed herein, as well as eukaryotic sources (see, for example, Peng et al., J. Bact., 139(2):339-45, 1979; Kline et al., J. Bact., 130(2):951-3, 1977, the entire contents of each of which are expressly incorporated herein by reference). branched chain amino acid aminotransferase enzymes are available from many microorganism sources, including those disclosed herein.

In some embodiments, the branched chain amino acid aminotransferase is encoded by at least one gene encoding a branched chain amino acid aminotransferase derived from a bacterial species. In some embodiments, the branched chain amino acid aminotransferase is encoded by at least one gene encoding a branched chain amino acid aminotransferase derived from a non-bacterial species. In some embodiments, the branched chain amino acid aminotransferase is encoded by at least one gene derived from a eukaryotic species, e.g., a yeast species or a plant species. In another embodiment, the branched chain amino acid aminotransferase is encoded by at least one gene derived from a mammalian species, e.g., human

In one embodiment, the at least one gene encoding the branched chain amino acid aminotransferase enzyme is derived from an organism of the genus or species that includes, but is not limited to, Arabidopsis, Bos, Brevibacillus, Canis, Corynebacterium, Cucumis, Deinococcus, Enterobacter, Entodinium, Escherichia, Gluconobacter, Helicobacter, Homo, Lactobacillus, Lactococcus, Macaca, Methanococcus, Mus, Mycobacterium, Neurospora, Nicotiana, Ovis, Pseudomonas, Rattus, Saccharomyces, Salmonella, Schizosaccharomyces, Solanum, Streptococcus, Sus, or Yarrowia species. In one embodiment, the branched chain amino acid aminotransferase is encoded by ilvE. In one embodiment, the ilvE gene is encoded by a gene derived from Arabidopsis thaliana, Bos taurus, Brevibacillus brevis, Brevibacterium flavum, Candida maltose, Canis lupus familiaris, Corynebacterium glutamicum, cucumis sativus, Deinococcus radiodurans, Enterobacter sp. TL3, enterococcus faecalis, Entodinium sp., Escherichia coli, gluconobacter oxydans, Helicobacter pylori, Homo sapiens, Lactobacillus paracasei, Lactococcus lactis, Macaca sp., Methanococcus aeolicus, Methanococcus maripaludis, Methanococcus voltae, Mus musculus, Mycobacterium smegmatis, Mycobacterium tuberculosis, Neurospora crassa, Nicotiana benthamiana, Ovis aries, Pseudomonas sp., Rattus norvegicus, Saccharomyces cerevisiae, Salmonella enterica, Schizosaccharomyces pombe, Solanum lycopersicum, Solanum pennellii, Staphylococcus camosus, Streptococcus mutans, Sus scrofa, or Yarowia lipolytica. In another embodiment, the branched chain amino acid aminotransferase, e.g., livE, is encoded by at least one gene derived from Escherichia coli. In one embodiment, the branched chain amino acid aminotransferase, e.g., livE, is encoded by at least gene from E. coli Nissle. In another embodiment, the branched chain amino acid aminotransferase, e.g., ilvE, is encoded by at least one gene derived from Lactobacillus lactis. In another embodiment, the, branched chain amino acid aminotransferase, e.g, ilvE, is encoded by at least one gene derived from Staphylococcus camosus. In some embodiments, the branched chain amino acid aminotransferase, e.g. ilvE, is encoded by at least one gene derived from Streptococcus mutans. In another embodiment, the branched chain amino acid aminotransferase, e.g., ilvE is encoded by at least one gene derived from Bacillus subtilis. In another embodiment, the branched chain amino acid aminotransferase, e.g., ilvE is encoded by at least one gene derived from Salmonella typhi.

In one embodiment, the at least one gene encoding the branched chain amino acid aminotransferase has been codon-optimized for use in the recombinant bacterial cell. In one embodiment, the at least one gene encoding the branched chain amino acid aminotransferase has been codon-optimized for use in Escherichia coli.

When a branched chain amino acid aminotransferase enzyme is expressed in the recombinant bacterial cells disclosed herein, the bacterial cells catabolize more branched chain amino acid, e.g., leucine, isoleucine, and/or valine, than unmodified bacteria of the same bacterial subtype under the same conditions (e.g., culture or environmental conditions). Thus, the genetically engineered bacteria comprising at least one heterologous gene encoding a branched chain amino acid aminotransferase may be used to catabolize excess branched chain amino acids, e.g., leucine, isoleucine, and/or valine, to treat a disease associated with the deamination of a branched chain amino acid, including Maple Syrup Urine Disease (MSUD).

The present disclosure further comprises genes encoding functional fragments of a branched chain amino acid aminotransferase enzyme, e.g., ilvE, or functional variants of branched chain amino acid aminotransferase enzyme, e.g., ilvE. The present disclosure encompasses genes encoding a branched chain amino acid aminotransferase enzyme, e.g., ilvE, comprising amino acids in its sequence that are substantially the same as an amino acid sequence described herein. As used herein, the term “functional fragment thereof” or “functional variant thereof” of a branched chain amino acid aminotransferase enzyme, e.g., ilvE, gene relates to a sequence having qualitative biological activity in common with the wild-type branched chain amino acid aminotransferase, e.g., ilvE, from which the fragment or variant was derived. For example, a functional fragment or a functional variant of a mutated branched chain amino acid aminotransferase protein, e.g., ilvE, is one which retains essentially the same ability to catabolize BCAAs as the branched chain amino acid aminotransferase protein, e.g., ilvE, from which the functional fragment or functional variant was derived. For example, a polypeptide having branched chain amino acid aminotransferase enzyme, e.g., ilvE, activity may be truncated at the N-terminus or C-terminus and the retention of branched chain amino acid aminotransferase enzyme, e.g., ilvE, activity assessed using assays known to those of skill in the art, including the exemplary assays provided herein. In one embodiment, the recombinant bacterial cell disclosed herein comprises a heterologous gene encoding an a branched chain amino acid aminotransferase enzyme, e.g., ilvE, functional variant. In another embodiment, the recombinant bacterial cell disclosed herein comprises a heterologous gene encoding a branched chain amino acid aminotransferase enzyme, e.g., ilvE, functional fragment.

Assays for testing the activity of a branched chain amino acid aminotransferase, a branched chain amino acid aminotransferase functional variant, or a branched chain amino acid aminotransferase functional fragment, e.g., ilvE, ilvE functional variant, and ilvE functional fragment are well known to one of ordinary skill in the art. For example, branched chain amino acid aminotransferase activity can be assessed by expressing the protein, functional variant, or fragment thereof, in a recombinant bacterial cell that lacks endogenous branched chain amino acid aminotransferase activity. Also, activity can be assessed using the enzymatic assay methods as described by Santiago et al. (J. Bacterial., 195(16):3552-62, 2013), the entire contents of which are expressly incorporated herein by reference.

In some embodiments, the at least one gene encoding a branched chain amino acid aminotransferase enzyme, e.g., ilvE, is mutagenized, mutants exhibiting increased activity are selected, and the mutagenized gene(s) encoding the branched chain amino acid aminotransferase enzyme, e.g., ilvE, are isolated and inserted into the bacterial cell. In some embodiments, the at least one gene encoding a branched chain amino acid aminotransferase enzyme, e.g., ilvE, is mutagenized, mutants exhibiting decreased activity are selected, and the mutagenized gene(s) encoding the branched chain amino acid aminotransferase enzyme, e.g., ilvE, are isolated and inserted into the bacterial cell. The gene comprising the modifications described herein may be present on a plasmid or chromosome.

In some embodiments, the branched chain amino acid aminotransferase is co-expressed with an additional branched chain amino acid catabolism enzyme. In some embodiments, the branched chain amino acid aminotransferase is co-expressed with an another branched chain amino acid deaminase enzyme, e.g. a BCAA dehydrogenase, such as leucine dehydrogenase, an AA aminotransferase, an amino acid oxidase, such as L-AAD. In some embodiments, the branched chain amino acid aminotransferase is co-expressed with one or more keto-acid decarboxylase enzyme(s), e.g., kivD. In some embodiments, the branched chain amino acid aminotransferase is co-expressed with one or more alcohol dehydrogenases, e.g., adh2 and/or yqhD. In some embodiments, the branched chain amino acid aminotransferase is co-expressed with one or more aldehyde dehydrogenases, e.g., padA. In some embodiments, the branched chain amino acid aminotransferase is co-expressed with one or more keto-acid decarboxylase enzymes, e.g., kivD and one or more alcohol dehydrogenase enzymes, e.g., adh2 or YqhD. In some embodiments, the branched chain amino acid aminotransferase is co-expressed with one or more keto-acid decarboxylase enzymes, e.g., kivD and one or more aldehyde dehydrogenase enzymes, e.g., padA. In some embodiments, the branched chain amino acid aminotransferase is co-expressed with one or more keto-acid decarboxylase enzymes, e.g., kivD, one or more aldehyde dehydrogenase enzymes, e.g., padA, and one or more alcohol dehydrogenase enzymes, e.g., adh2 or YqhD, each of which are described in more detail herein. In some embodiments, the branched chain amino acid aminotransferase is co-expressed with another branched chain amino acid deaminase enzyme, e.g. a BCAA dehydrogenase, such as leucine dehydrogenase, a BCAA aminotransferase, and/or an amino acid oxidase, such as L-AAD and is co-expressed with one or more keto-acid decarboxylase enzyme(s), e.g., kivD. In some embodiments, the branched chain amino acid aminotransferase, is co-expressed with another branched chain amino acid deaminase enzyme, e.g. a BCAA dehydrogenase, such as leucine dehydrogenase, a BCAA aminotransferase, and/or an amino acid oxidase, such as L-AAD and is co-expressed with one or more alcohol dehydrogenases, e.g., adh2 and/or yqhD. In some embodiments, the branched chain amino acid aminotransferase is co-expressed with another branched chain amino acid deaminase enzyme, e.g. a BCAA dehydrogenase, such as leucine dehydrogenase, a BCAA aminotransferase, and/or an amino acid oxidase, such as L-AAD and is co-expressed with one or more aldehyde dehydrogenases, e.g., padA. In some embodiments, the branched chain amino acid aminotransferase is co-expressed with another branched chain amino acid deaminase enzyme, e.g. a BCAA dehydrogenase, such as leucine dehydrogenase, a BCAA aminotransferase, and/or an amino acid oxidase, such as L-AAD, is co-expressed with one or more keto-acid decarboxylase enzyme(s), e.g., kivD, and co-expressed with one or more alcohol dehydrogenases, e.g., adh2 and/or yqhD and/or one or more aldehyde dehydrogenases, e.g., padA. In some embodiments, the at least one branched chain amino acid aminotransferase enzyme is coexpressed with one or more BCAA transporter(s), for example, a high affinity leucine transporter, e.g., LivKHMGF and/or low affinity BCAA transporter BrnQ. In some embodiments, the at least one branched chain amino acid aminotransferase enzyme is coexpressed with one or more BCAA binding protein(s), for example, LivJ. In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one branched chain amino acid aminotransferase enzyme and genetic modification that reduces export of a branched chain amino acid, e.g., a genetic mutation in a leuE gene or promoter thereof. In one embodiment, the engineered bacteria comprise gene sequence(s) encoding at least one branched chain amino acid aminotransferase enzyme and a genetic modification that reduces or eliminates branched chain amino acid synthesis, e.g., a genetic mutation in a ilvC gene or promoter thereof.

In some embodiments, the engineered bacteria comprise gene sequence(s) encoding at least one branched chain amino acid aminotransferase enzyme and further comprise gene sequence encoding one or more polypeptides selected from other branched chain amino acid catabolism enzyme(s), BCAA transporter(s), and BCAA binding protein(s). In some embodiments, the engineered bacteria comprise gene sequence(s) encoding at least one branched chain amino acid aminotransferase enzyme, e.g., ilvE, and gene sequence(s) encoding one or more branched chain amino acid dehydrogenase(s) (e.g., leuDH). In some embodiments, the engineered bacteria comprise gene sequence(s) encoding at least one branched chain amino acid aminotransferase enzyme, e.g., ilvE, and gene sequence(s) encoding one or more amino acid oxidase(s) (e.g. L-AAD)). In some embodiments, the engineered bacteria comprise gene sequence(s) encoding at least one branched chain amino acid aminotransferase enzyme, ilvE, and gene sequence(s) encoding one or more other aminotransferase(s). In some embodiments, the engineered bacteria comprise gene sequence(s) encoding at least one branched chain amino acid aminotransferase enzyme, e.g., ilvE, and gene sequence(s) encoding one or more keto-acid decarboxylase enzyme(s), e.g., kivD. In some embodiments, the engineered bacteria comprise gene sequence(s) encoding at least one branched chain amino acid aminotransferase enzyme, e.g., ilvE, and gene sequence(s) encoding one or more aldehyde dehydrogenase(s) (e.g., padA). In some embodiments, the engineered bacteria comprise gene sequence(s) encoding at least one branched chain amino acid aminotransferase enzyme, e.g., ilvE, and gene sequence(s) encoding one or more alcohol dehydrogenase(s) (e.g., adh2, yqhD).

In some embodiments, the engineered bacteria comprise gene sequence(s) encoding at least one branched chain amino acid aminotransferase enzyme, e.g., ilvE, and gene sequence(s) encoding one or more other branched chain amino acid catabolism enzyme(s), for example, branched chain amino acid dehydrogenase(s), such as leuDH, branched chain amino acid aminotransferase enzyme, and/or amino acid oxidase(s), such as L-AAD and gene sequence(s) encoding one or more keto-acid decarboxylase enzyme(s), e.g., kivD. In some embodiments, the engineered bacteria comprise gene sequence(s) encoding at least one branched chain amino acid aminotransferase enzyme, e.g., ilvE, and gene sequence(s) encoding one or more other branched chain amino acid catabolism enzyme(s), for example, branched chain amino acid dehydrogenase(s), such as leuDH, branched chain amino acid aminotransferase enzyme, and/or amino acid oxidase(s), such as L-AAD, gene sequence(s) encoding one or more keto-acid decarboxylase enzyme(s), e.g., kivD, and gene sequence(s) encoding one or more aldehyde dehydrogenase(s) (e.g., padA) and/or gene sequence(s) encoding one or more alcohol dehydrogenase(s) (e.g., adh2, yqhD).

In some embodiments, the engineered bacteria comprise gene sequence(s) encoding at least one branched chain amino acid aminotransferase enzyme, e.g., ilvE, and gene sequence(s) encoding one or more BCAA transporter(s) (e.g., LivKHMGF, brnQ). In some embodiments, the engineered bacteria comprise gene sequence(s) encoding at least one branched chain amino acid aminotransferase enzyme, e.g. ilvE, and gene sequence(s) encoding one or more BCAA binding protein(s) (e.g., livJ). In some embodiments, the engineered bacteria comprise gene sequence(s) encoding at least one branched chain amino acid aminotransferase enzyme, e.g. ilvE, gene sequence(s) encoding one or more BCAA transporter(s) (e.g., LivKHMGF, brnQ), and gene sequence(s) encoding one or more BCAA binding protein(s) (e.g., livJ). In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one branched chain amino acid aminotransferase enzyme, e.g. ilvE, and genetic modification that reduces export of a branched chain amino acid, e.g., a genetic mutation in a leuE gene or promoter thereof. In one embodiment, the engineered bacteria comprise gene sequence(s) encoding at least one branched chain amino acid aminotransferase enzyme, e.g. ilvE, and a genetic modification that reduces or eliminates branched chain amino acid synthesis, e.g., a genetic mutation in a ilvC gene or promoter thereof.

In some embodiments, the gene sequence(s) encoding the one or more branched chain amino acid aminotransferase enzyme(s), e.g. ilvE, is expressed under the control of a constitutive promoter. In some embodiments, the gene sequence(s) encoding the one or more branched chain amino acid aminotransferase enzyme(s), e.g. ilvE, is expressed under the control of an inducible promoter. In some embodiments, the gene sequence(s) encoding the one or more branched chain amino acid aminotransferase enzyme(s), e.g. ilvE, is expressed under the control of a promoter that is directly or indirectly induced by exogenous environmental conditions. In some embodiments, the gene sequence(s) encoding the one or more branched chain amino acid aminotransferase enzyme(s), e.g. ilvE, is expressed under the control of a promoter that is directly or indirectly induced by low-oxygen or anaerobic conditions, wherein expression of the gene encoding the branched chain amino acid aminotransferase enzyme, e.g. ilvE, is activated under low-oxygen or anaerobic environments, such as the environment of the mammalian gut. In some embodiments, the gene sequence(s) encoding the one or more branched chain amino acid aminotransferase enzyme, e.g. ilvE, is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

In some embodiments, the at least one gene encoding the branched chain amino acid aminotransferase comprises the ilvE gene. In a specific embodiment, the ilvE gene has at least about 80% identity with the sequence of SEQ ID NO:22. In one embodiment, the ilvE gene has at least about 90% identity with the sequence of SEQ ID NO:22. In one embodiment, the ilvE gene has at least about 95% identity with the sequence of SEQ ID NO:22. In another embodiment, the ilvE gene has at least about 85%, 86%, 87%, 88%, 89%, 90%, 91%, 92%, 93%, 94%, 95%, 96%, 97%, 98%, or 99% identity with the sequence of SEQ ID NO:22. In another embodiment, the ilvE gene comprises the sequence of SEQ ID NO:22. In yet another embodiment, the ilvE gene consists of the sequence of SEQ ID NO:22.

Amino Acid Oxidase/Amino Acid Deaminase

In other embodiments, the branched chain amino acid deamination enzyme is a branched chain amino acid oxidase (also referred as branched chain amino acid deaminase). In some embodiments, the engineered bacteria comprise gene sequence(s) encoding one or more copies of a branched chain amino acid oxidase. In some embodiments, the engineered bacteria comprise gene sequence(s) encoding one, two, three, four, five, six, or more copies of a branched chain amino acid oxidase. The one or more copies of a branched chain amino acid oxidase can be one or more copies of the same gene or can be different genes encoding branched chain amino acid oxidase, e.g., gene(s) from a different species or otherwise having a different gene sequence. The one or more copies of branched chain amino acid oxidase can be present in the bacterial chromosome or can be present in one or more plasmids. As used herein “branched chain amino acid oxidase” refers to any polypeptide having enzymatic activity that deaminates a branched chain amino acid, e.g., leucine, to its corresponding ketoacid, e.g., alpha-ketoisocaproate (KIC) (EC 1.4.3.2). Multiple distinct branched chain amino acid aminotransferases are known in the art and are available from many microorganism sources, including those disclosed herein, as well as eukaryotic sources (see, for example, Song et al., Scientific Reports, Nature, 5:12694; DOI: 10:1038/srep12694 (2015)) the entire contents of each of which are expressly incorporated herein by reference).

In some embodiments, the branched chain amino acid oxidase is encoded by at least one gene encoding a branched chain amino acid oxidase derived from a bacterial species. In some embodiments, the branched chain amino acid oxidase is encoded by at least one gene encoding a branched chain amino acid oxidase derived from a non-bacterial species. In some embodiments, the branched chain amino acid oxidase is encoded by at least one gene derived from a eukaryotic species, e.g., a yeast species or a plant species. In another embodiment, the branched chain amino acid oxidase is encoded by at least one gene derived from a mammalian species, e.g., a human.

In some embodiments, the at least one gene encoding the branched chain amino acid oxidase enzyme is derived from an organism of the genus or species that includes, but is not limited to, Arabidopsis, Bos, Brevibacillus, Canis, Corynebacterium, Cucumis, Deinococcus, Enterobacter, Entodinium, Escherichia, Gluconobacter, Helicobacter, Homo, Lactobacillus, Lactococcus, Macaca, Methanococcus, Mus, Mycobacterium, Neurospora, Nicotiana, Ovis, Pseudomonas, Rattus, Saccharomyces, Salmonella, Schizosaccharomyces, Solanum, Streptococcus, Sus, or Yarrowia species. In one embodiment, the branched chain amino acid aminotransferase is encoded by L-AAD. In one embodiment, the L-AAD gene is encoded by a gene derived from Arabidopsis thaliana, Bos taurus, Brevibacillus brevis, Brevibacterium flavum, Candida maltose, Canis lupus familiaris, Corynebacterium glutamicum, cucumis sativus, Deinococcus radiodurans, Enterobacter sp. TL3, enterococcus faecalis, Entodinium sp., Escherichia coli, gluconobacter oxydans, Helicobacter pylori, Homo sapiens, Lactobacillus paracasei, Lactococcus lactis, Macaca sp., Methanococcus aeolicus, Methanococcus maripaludis, Methanococcus voltae, Mus musculus, Mycobacterium smegmatis, Mycobacterium tuberculosis, Neurospora crassa, Nicotiana benthamiana, Ovis aries, Pseudomonas sp., Rattus norvegicus, Saccharomyces cerevisiae, Salmonella enterica, Schizosaccharomyces pombe, Solanum lycopersicum, Solanum pennellii, Staphylococcus carnosus, Streptococcus mutans, Sus scrofa, or Yarowia lipolytica. In another embodiment, the L-AAD is encoded by at least one gene derived from Lactobacillus lactis. In another embodiment, the L-AAD is encoded by at least one gene derived from Staphylococcus carnosus. In some embodiments, the L-AAD is encoded by at least one gene derived from Streptococcus mutans. In another embodiment, the L-AAD is encoded by at least one gene derived from Bacillus subtilis. In another embodiment, the L-AAD is encoded by at least one gene derived from Salmonella typhi. In another embodiment, the L-AAD is encoded by at least one gene derived from Proteus vulgaris. In another embodiment, the L-AAD is encoded by at least one gene derived from Proteus mirabilis.

Substrate specificities of selected Proteus L-amino acid deaminases are shown in Table 3 and are described Baek et al., Journal of Basic Microbiology 2011, 51, 129-135; “Expression and characterization of a second L-amino acid deaminase isolated from Proteus mirabilis in Escherichia coli”, the contents of which is herein incorporated by reference in its entirety. Two LAADs exist in P. mirabilis. In certain embodiments of the disclosure, LAAD(Pv) refers to Pma. The amino acid deaminase activities are presented as percentages of the activities against amino acid deaminases, respectively, only perpendicularly.

TABLE 3
Substrate specificities of selected amino
acid deaminases from Proteus species

| AA | Pm1 | LAD | Pma |
|---|---|---|---|
| Ala | 9 | 3.5 | 0.6 |
| Arg | 51.2 | 27.3 | 28.2 |
| Asn | 5.2 | 43.6 | 0 |
| Asp | 2.6 | 55.4 | 10.9 |
| Cys | 9 | — | 1.9 |
| Gln | 5.2 | 1.1 | 1.3 |
| Glu | 35.8 | 1.1 | 0.6 |
| Gly | 7.6 | — | 1.3 |
| His | 100 | 79.9 | 0 |
| Ilu | 6.4 | — | 2.6 |
| Leu | 7.6 | 105 | 41.7 |
| Lys | 7.6 | 3.5 | 1.9 |
| Met | 2.6 | 100 | 16.7 |
| Phe | 46.2 | 37.4 | 100 |
| Pro | 14.2 | 0.7 | 3.2 |
| Ser | 3.8 | — | 1.3 |
| Thr | 12.8 | 1.1 | 0 |
| Trp | 10.2 | 41.6 | 3.2 |
| Tyr | 9 | 92.8 | 0.6 |
| Val | 6.4 | — | 1.3 |

Pm1: amino acid deaminase gene from P. mirabilis KCTC 2566 (Genbank: EU669819.1)
LAD: L-amino acid deaminase of P. vulgaris (Genbank: AB030003)
Pma: amino acid deaminase gene from P. mirabilis (Genbank: U35383)

In one embodiment, the at least one gene encoding the branched chain amino acid oxidase has been codon-optimized for use in the recombinant bacterial cell. In one embodiment, the at least one gene encoding the branched chain amino acid oxidase has been codon-optimized for use in Escherichia coli.

When a branched chain amino acid oxidase enzyme is expressed in the recombinant bacterial cells disclosed herein, the bacterial cells catabolize more branched chain amino acid, e.g., leucine, isoleucine, and/or valine, than unmodified bacteria of the same bacterial subtype under the same conditions (e.g., culture or environmental conditions). Thus, the genetically engineered bacteria comprising at least one heterologous gene encoding a branched chain amino acid oxidase may be used to catabolize excess branched chain amino acids, e.g., leucine, isoleucine, and/or valine, to treat a disease associated with the deamination of a branched chain amino acid, including Maple Syrup Urine Disease (MSUD).

The present disclosure further comprises genes encoding functional fragments of a branched chain amino acid oxidase enzyme, e.g., L-AAD, or functional variants of branched chain amino acid oxidase enzyme, e.g., L-AAD. The present disclosure encompasses genes encoding a branched chain amino acid oxidase enzyme, e.g., L-AAD, comprising amino acids in its sequence that are substantially the same as an amino acid sequence described herein. As used herein, the term “functional fragment thereof” or “functional variant thereof” of a branched chain amino acid oxidase enzyme, e.g., L-AAD, gene relates to a sequence having qualitative biological activity in common with the wild-type branched chain amino acid oxidase, e.g., L-AAD, from which the fragment or variant was derived. For example, a functional fragment or a functional variant of a mutated branched chain amino acid oxidase protein, e.g., L-AAD, is one which retains essentially the same ability to catabolize BCAAs as branched chain amino acid oxidase protein, e.g., L-AAD, from which the functional fragment or functional variant was derived. For example, a polypeptide having branched chain amino acid oxidase enzyme, e.g., L-AAD, activity may be truncated at the N-terminus or C-terminus and the retention branched chain amino acid oxidase enzyme, e.g., L-AAD, activity assessed using assays known to those of skill in the art, including the exemplary assays provided herein. In one embodiment, the recombinant bacterial cell disclosed herein comprises a heterologous gene encoding an a branched chain amino acid oxidase enzyme, e.g., L-AAD, functional variant. In another embodiment, the recombinant bacterial cell disclosed herein comprises a heterologous gene encoding branched chain amino acid oxidase enzyme, e.g., L-AAD, functional fragment.

Assays for testing the activity of a branched chain amino acid oxidase, a branched chain amino acid oxidase functional variant, or a branched chain amino acid oxidase functional fragment are well known to one of ordinary skill in the art. For example, branched chain amino acid oxidase activity can be assessed by expressing the protein, functional variant, or functional fragment thereof, in a recombinant bacterial cell that lacks endogenous branched chain amino acid oxidase activity. Also, activity can be assessed using the enzymatic assay methods as described by Santiago et al. (J. Bacterial., 195(16):3552-62, 2013), the entire contents of which are expressly incorporated herein by reference.

In some embodiments, the at least one gene encoding a branched chain amino acid oxidase is mutagenized, mutants exhibiting increased activity are selected, and the mutagenized gene(s) encoding the branched chain amino acid oxidase are isolated and inserted into the bacterial cell. In some embodiments, the at least one gene encoding the branched chain amino acid oxidase is mutagenized, mutants exhibiting decreased activity are selected, and the mutagenized gene(s) encoding the branched chain amino acid oxidase are isolated and inserted into the bacterial cell. The gene comprising the modifications described herein may be present on a plasmid or chromosome.

In some embodiments, the branched chain amino acid oxidase is co-expressed with an additional branched chain amino acid catabolism enzyme. In some embodiments, the branched chain amino acid oxidase is co-expressed with one or more additional branched chain amino acid catabolism enzyme(s) selected from a branched chain amino acid deaminase enzyme, e.g. a BCAA dehydrogenase, such as leucine dehydrogenase; a BCAA aminotransferase, e.g., ilvE, and a branched chain amino acid oxidase, e.g., L-AAD. In some embodiments, the branched chain amino acid oxidase is co-expressed with one or more keto-acid decarboxylase enzyme(s), e.g., kivD. In some embodiments, the branched chain amino acid oxidase is co-expressed with one or more alcohol dehydrogenases, e.g., adh2 and/or yqhD. In some embodiments, the branched chain amino acid oxidase is co-expressed with one or more aldehyde dehydrogenases, e.g., padA. In some embodiments, the branched chain amino acid oxidase is co-expressed with one or more keto-acid decarboxylase enzymes, e.g., kivD and one or more alcohol dehydrogenase enzymes, e.g., adh2 or YqhD. In some embodiments, the branched chain amino acid oxidase is co-expressed with one or more keto-acid decarboxylase enzymes, e.g., kivD and one or more aldehyde dehydrogenase enzymes, e.g., padA. In some embodiments, the branched chain amino acid oxidase is co-expressed with one or more keto-acid decarboxylase enzymes, e.g., kivD, one or more aldehyde dehydrogenase enzymes, e.g., padA, and one or more alcohol dehydrogenase enzymes, e.g., adh2 or YqhD, each of which are described in more detail herein.

In some embodiments, the branched chain amino acid oxidase is co-expressed with another branched chain amino acid deaminase enzyme, e.g. a BCAA dehydrogenase, such as leucine dehydrogenase, a BCAA aminotransferase, e.g., ilvE, and/or another amino acid oxidase and is co-expressed with one or more keto-acid decarboxylase enzyme(s), e.g., kivD. In some embodiments, branched chain amino acid oxidase, is co-expressed with another branched chain amino acid deaminase enzyme, e.g. a BCAA dehydrogenase, such as leucine dehydrogenase, a BCAA aminotransferase, e.g., ilvE, and/or another amino acid oxidase and is co-expressed with one or more alcohol dehydrogenases, e.g., adh2 and/or yqhD. In some embodiments, the branched chain amino acid oxidase is co-expressed with another branched chain amino acid deaminase enzyme, e.g. a BCAA dehydrogenase, such as leucine dehydrogenase, a BCAA aminotransferase, e.g., ilvE, and/or another amino acid oxidase and is co-expressed with one or more aldehyde dehydrogenases, e.g., padA. In some embodiments, the branched chain amino acid oxidase is co-expressed with another branched chain amino acid deaminase enzyme, e.g. a BCAA dehydrogenase, such as leucine dehydrogenase, a BCAA aminotransferase, e.g., ilvE, and/or another amino acid oxidase, is co-expressed with one or more keto-acid decarboxylase enzyme(s), e.g., kivD, and co-expressed with one or more alcohol dehydrogenases, e.g., adh2 and/or yqhD and/or one or more aldehyde dehydrogenases, e.g., padA. In some embodiments, the at least one branched chain amino acid oxidase enzyme is coexpressed with one or more BCAA transporter(s), for example, a high affinity leucine transporter, e.g., LivKHMGF and/or low affinity BCAA transporter BrnQ. In some embodiments, the at least one branched chain amino acid oxidase enzyme is coexpressed with one or more BCAA binding protein(s), for example, LivJ. In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one branched chain amino acid oxidase enzyme and genetic modification that reduces export of a branched chain amino acid, e.g., a genetic mutation in a leuE gene or promoter thereof. In one embodiment, the engineered bacteria comprise gene sequence(s) encoding at least one branched chain amino acid oxidase enzyme and a genetic modification that reduces or eliminates branched chain amino acid synthesis, e.g., a genetic mutation in a ilvC gene or promoter thereof.

In some embodiments, the engineered bacteria comprise gene sequence(s) encoding at least one branched chain amino acid oxidase enzyme and further comprise gene sequence encoding one or more polypeptides selected from other branched chain amino acid catabolism enzyme(s), BCAA transporter(s), and BCAA binding protein(s). In some embodiments, the engineered bacteria comprise gene sequence(s) encoding at least one branched chain amino acid oxidase enzyme, e.g., L-AAD, and gene sequence(s) encoding one or more branched chain amino acid dehydrogenase(s) (e.g., leuDH). In some embodiments, the engineered bacteria comprise gene sequence(s) encoding at least one branched chain amino acid oxidase enzyme, e.g., L-AAD, and gene sequence(s) encoding one or more other amino acid oxidase(s). In some embodiments, the engineered bacteria comprise gene sequence(s) encoding at least one branched chain amino acid oxidase enzyme, L-AAD, and gene sequence(s) encoding one or more BCAA aminotransferase(s), e.g., ilvE. In some embodiments, the engineered bacteria comprise gene sequence(s) encoding at least one branched chain amino acid oxidase enzyme, e.g., L-AAD, and gene sequence(s) encoding one or more keto-acid decarboxylase enzyme(s), e.g., kivD. In some embodiments, the engineered bacteria comprise gene sequence(s) encoding at least one branched chain amino acid oxidase enzyme, e.g., L-AAD, and gene sequence(s) encoding one or more aldehyde dehydrogenase(s) (e.g., padA). In some embodiments, the engineered bacteria comprise gene sequence(s) encoding at least one branched chain amino acid oxidase enzyme, e.g., L-AAD, and gene sequence(s) encoding one or more alcohol dehydrogenase(s) (e.g., adh2, yqhD).

In some embodiments, the engineered bacteria comprise gene sequence(s) encoding at least one branched chain amino acid oxidase enzyme, e.g., L-AAD, and gene sequence(s) encoding one or more other branched chain amino acid catabolism enzyme(s), for example, branched chain amino acid dehydrogenase(s), such as leuDH, branched chain amino acid aminotransferase enzyme, e.g., ilvE, and/or another amino acid oxidase(s) and gene sequence(s) encoding one or more keto-acid decarboxylase enzyme(s), e.g., kivD. In some embodiments, the engineered bacteria comprise gene sequence(s) encoding at least one branched chain amino acid oxidase enzyme, e.g., L-AAD, and gene sequence(s) encoding one or more other branched chain amino acid catabolism enzyme(s), for example, branched chain amino acid dehydrogenase(s), such as leuDH, branched chain amino acid aminotransferase enzyme, e.g., ilvE, and/or another amino acid oxidase(s), gene sequence(s) encoding one or more keto-acid decarboxylase enzyme(s), e.g., kivD, and gene sequence(s) encoding one or more aldehyde dehydrogenase(s) (e.g., padA) and/or gene sequence(s) encoding one or more alcohol dehydrogenase(s) (e.g., adh2, yqhD).

In some embodiments, the engineered bacteria comprise gene sequence(s) encoding at least one branched chain amino acid oxidase enzyme, e.g., L-AAD, and gene sequence(s) encoding one or more BCAA transporter(s) (e.g., LivKHMGF, brnQ). In some embodiments, the engineered bacteria comprise gene sequence(s) encoding at least one branched chain amino acid oxidase enzyme, e.g. L-AAD, and gene sequence(s) encoding one or more BCAA binding protein(s) (e.g., livJ). In some embodiments, the engineered bacteria comprise gene sequence(s) encoding at least one branched chain amino acid oxidase enzyme, e.g. L-AAD, gene sequence(s) encoding one or more BCAA transporter(s) (e.g., LivKHMGF, brnQ), and gene sequence(s) encoding one or more BCAA binding protein(s) (e.g., livJ). In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one branched chain amino acid oxidase enzyme, e.g. L-AAD, and genetic modification that reduces export of a branched chain amino acid, e.g., a genetic mutation in a leuE gene or promoter thereof. In one embodiment, the engineered bacteria comprise gene sequence(s) encoding at least one branched chain amino acid oxidase enzyme, e.g. L-AAD, and a genetic modification that reduces or eliminates branched chain amino acid synthesis, e.g., a genetic mutation in a ilvC gene or promoter thereof.

In some embodiments, the gene sequence(s) encoding the one or more branched chain amino acid oxidase enzyme(s), e.g. L-AAD, is expressed under the control of a constitutive promoter. In some embodiments, the gene sequence(s) encoding the one or more branched chain amino acid oxidase enzyme(s), e.g. L-AAD, is expressed under the control of an inducible promoter. In some embodiments, the gene sequence(s) encoding the one or more branched chain amino acid oxidase enzyme(s), e.g. L-AAD, is expressed under the control of a promoter that is directly or indirectly induced by exogenous environmental conditions. In some embodiments, the gene sequence(s) encoding the one or more branched chain amino acid oxidase enzyme(s), e.g. L-AAD, is expressed under the control of a promoter that is directly or indirectly induced by low-oxygen or anaerobic conditions, wherein expression of the gene encoding the branched chain amino acid oxidase, e.g. L-AAD, is activated under low-oxygen or anaerobic environments, such as the environment of the mammalian gut. In some embodiments, the gene sequence(s) encoding the one or more branched chain amino acid oxidase enzyme(s), e.g. L-AAD, is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Non-limiting examples of inducible promoters include, but are not limited to, an FNR responsive promoter, a P_{azaC} promoter, a P_{araBAD} promoter, and a P_{TetR} promoter, each of which are described in more detail herein. Other inducible promoters are discussed herein and otherwise known in the art.

In one embodiment, the at least one gene encoding the branched chain amino acid oxidase comprises the L-AAD gene. In one embodiment, the L-AAD gene has at least about 80% identity with the sequence of SEQ ID NO:24, SEQ ID NO:26, and/or SEQ ID NO:56. In one embodiment, the L-AAD gene has at least about 90% identity with the sequence of SEQ ID NO:24, SEQ ID NO:26, and/or SEQ ID NO:56. In one embodiment, the L-AAD gene has at least about 95% identity with the sequence of SEQ ID NO:24, SEQ ID NO:26, and/or SEQ ID NO:56. In another embodiment, the L-AAD gene has at least about 85%, 86%, 87%, 88%, 89%, 90%, 91%, 92%, 93%, 94%, 95%, 96%, 97%, 98%, or 99% identity with the sequence of SEQ ID NO:24, SEQ ID NO:26, and/or SEQ ID NO:56. In another embodiment, the L-AAD gene comprises the sequence of SEQ ID NO:24, SEQ ID NO:26, and/or SEQ ID NO:56. In yet another embodiment, the L-AAD gene consists of the sequence of SEQ ID NO:24, SEQ ID NO:26, and/or SEQ ID NO:56.

D. Alcohol and Aldehyde Dehydrogenase Enzymes

In some embodiments, wherein a branched chain amino acid catabolism enzyme is used to convert a ketoacid to its corresponding aldehyde, the recombinant bacterial cells may further comprise an alcohol dehydrogenase enzyme in order to convert the branched chain amino acid-derived aldehyde to its respective alcohol. In some embodiments, the engineered bacteria comprise gene sequence(s) encoding one or more copies of an alcohol dehydrogenase. In some embodiments, the engineered bacteria comprise gene sequence(s) encoding one, two, three, four, five, six, or more copies of an alcohol dehydrogenase. The one or more copies of an alcohol dehydrogenase can be one or more copies of the same gene or can be different genes encoding alcohol dehydrogenase, e.g., gene(s) from a different species or otherwise having a different gene sequence. The one or more copies of alcohol dehydrogenase can be present in the bacterial chromosome or can be present in one or more plasmids. As used herein, “alcohol dehydrogenase” refers to any polypeptide having enzymatic activity that catalyzes the conversion of a branched chain amino acid-derived aldehyde, e.g., isovaleraldehyde, isobutyraldehyde, and 2-methylbutyraldehyde, into its respective alcohol, e.g., isopentanol, isobutanol, and 2-methylbutanol.

In general, alcohol dehydrogenases (EC 1.1.1.1) belong to a group of dehydrogenase enzymes that facilitate the interconversion between alcohols and aldehydes or ketones with the reduction of nicotinamide adenine dinucleotide (NAD+ to NADH). Multiple distinct alcohol dehydrogenases are known in the art and are available from many microorganism sources, including those disclosed herein, as well as eukaryotic and plant sources (see, for example, Bennetzen et al., J. Biol. Chem., 257(6):3018-25, 1982 and Teng et al., Human Genetics, 53(1):87-90, 1979, the entire contents of each of which are expressly incorporated herein by reference).

In some embodiments, the alcohol dehydrogenase is encoded by at least one gene derived from a bacterial species. In some embodiments, the alcohol dehydrogenase is encoded by at least one gene derived from a non-bacterial species. In some embodiments, the alcohol dehydrogenase is encoded by at least one gene derived from a eukaryotic species, e.g., a yeast species or a plant species. In another embodiment, the alcohol dehydrogenase is encoded by at least one gene derived from a mammalian species, e.g., human.

In one embodiment, the at least one gene encoding the alcohol dehydrogenase is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Proteus, Pseudomonas, Psychrobacter, Ralstonia, Saccharomyces, Salmonella, Sarcina, Serratia, Staphylococcus, Streptococcus, and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Enterococcus faecalis, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Pseudomonas putida, Pseudomonas aeruginosa, Psychrobacter anticus, Proteus vulgaris, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Streptococcus faecalis, Yersinia enterocolitica, Yersinia mollaretii, Yersinia kristensenii, Yersinia rohdei, and Yersinia aldovae. In some embodiments, the alcohol dehydrogenase is selected from adh2 and yqhD. In some embodiments, the alcohol dehydrogenase, e.g., adh2 and yqhD, is encoded by at least one gene derived from Saccharomyces cerevisiae. In another embodiment, the alcohol dehydrogenase, e.g., adh2 and yqhD, is encoded by at least one gene derived from E. coli. In another embodiment, the alcohol dehydrogenase, e.g., adh2 and yqhD, is encoded by at least one gene derived from Oryza sativa. In another embodiment, the alcohol dehydrogenase, e.g., adh2 and yqhD, is encoded by at least one gene derived from Penicillium brasilianum. In another embodiment, the alcohol dehydrogenase, e.g., adh2 and yqhD, is encoded by at least one gene derived from Bifidobacterium longum.

In some embodiments, the at least one gene encoding the alcohol dehydrogenase has been codon-optimized for use in the recombinant bacterial cell. In some embodiments, the at least one gene encoding the alcohol dehydrogenase has been codon-optimized for use in Escherichia coli. For example, SEQ ID NOs:39 and 41 are codon-optimized sequences for adh2 and adh6, respectively.

In some embodiments, the at least one gene encoding the alcohol dehydrogenase is the human alcohol dehydrogenase (ADH1A, ADH1C2, ADH1B1). In another embodiment, the at least one gene encoding the alcohol dehydrogenase comprises the human ADH1α, ADH1β and ADH1 γ subunits. In another embodiment, the at least one gene encoding the alcohol dehydrogenase catalyzes the oxidation of ethanol to acetaldehyde. Niederhut, et al., Protein science, 10:697-706 (2001).

When an alcohol dehydrogenase is expressed in the recombinant bacterial cells disclosed herein, the bacterial cells convert more branched chain amino acid-derived aldehydes to their respective alcohols than unmodified bacteria of the same bacterial subtype under the same conditions (e.g., culture or environmental conditions). Thus, the genetically engineered bacteria comprising at least one heterologous gene encoding an alcohol dehydrogenase may be used to catabolize excess branched chain amino acid-derived aldehydes, e.g., isovaleraldehyde, to treat a disease associated with a branched chain amino acid, including Maple Syrup Urine Disease (MSUD).

The present disclosure encompasses genes encoding an alcohol dehydrogenase comprising amino acids in its sequence that are substantially the same as an amino acid sequence described herein. The present disclosure further comprises genes encoding functional fragments of an alcohol dehydrogenase or functional variants of an alcohol dehydrogenase. As used herein, the term “functional fragment thereof” or “functional variant thereof” of an alcohol dehydrogenase gene relates to a sequence having qualitative biological activity in common with the wild-type alcohol dehydrogenase, from which the fragment or variant was derived. For example, a functional fragment or a functional variant of a mutated alcohol dehydrogenase is one which retains essentially the same ability to catabolize BCAAs as alcohol dehydrogenase from which the functional fragment or functional variant was derived. For example, a polypeptide having alcohol dehydrogenase activity may be truncated at the N-terminus or C-terminus and the retention alcohol dehydrogenase activity assessed using assays known to those of skill in the art, including the exemplary assays provided herein. In one embodiment, the recombinant bacterial cell disclosed herein comprises a heterologous gene encoding an an alcohol dehydrogenase functional variant. In another embodiment, the recombinant bacterial cell disclosed herein comprises a heterologous gene encoding alcohol dehydrogenase functional fragment.

In some embodiments, the at least one gene encoding an alcohol dehydrogenase is mutagenized, mutants exhibiting increased activity are selected, and the mutagenized gene(s) encoding the alcohol dehydrogenase are isolated and inserted into the bacterial cell. In some embodiments, the at least one gene encoding the alcohol dehydrogenase is mutagenized, mutants exhibiting decreased activity are selected, and the mutagenized gene(s) encoding the alcohol dehydrogenase are isolated and inserted into the bacterial cell. The gene comprising the modifications described herein may be present on a plasmid or chromosome.

Assays for testing the activity of an alcohol dehydrogenase, an alcohol dehydrogenase functional variant, or an alcohol dehydrogenase functional fragment are well known to one of ordinary skill in the art. For example, alcohol dehydrogenase activity can be assessed by expressing the protein, functional variant, or fragment thereof, in a recombinant bacterial cell that lacks endogenous alcohol dehydrogenase activity. Also, activity can be assessed using the enzymatic assay methods as described by Kagi et al. (J. Biol. Chem., 235:3188-92, 1960), and Walker (Biochem. Educ., 20(1):42-43, 1992).

In some embodiments, the alcohol dehydrogenase is co-expressed with an additional branched chain amino acid catabolism enzyme. In some embodiments, the alcohol dehydrogenase is co-expressed with one or more additional branched chain amino acid catabolism enzyme(s) selected from a branched chain amino acid deaminase enzyme, e.g. a BCAA dehydrogenase, such as leucine dehydrogenase; a BCAA aminotransferase, e.g., ilvE, and a branched chain amino acid oxidase, e.g., L-AAD. In some embodiments, the alcohol dehydrogenase is co-expressed with one or more keto-acid decarboxylase enzyme(s), e.g., kivD. In some embodiments, the alcohol dehydrogenase is co-expressed with one or more other alcohol dehydrogenases. In some embodiments, the branched chain amino acid oxidase is co-expressed with one or more aldehyde dehydrogenases, e.g., padA. In some embodiments, the alcohol dehydrogenase is co-expressed with one or more keto-acid decarboxylase enzymes, e.g., kivD and one or more other alcohol dehydrogenase enzymes. In some embodiments, the alcohol dehydrogenase is co-expressed with one or more keto-acid decarboxylase enzymes, e.g., kivD and one or more aldehyde dehydrogenase enzymes, e.g., padA. In some embodiments, the alcohol dehydrogenase is co-expressed with one or more keto-acid decarboxylase enzymes, e.g., kivD, one or more aldehyde dehydrogenase enzymes, e.g., padA, and one or more other alcohol dehydrogenase enzymes.

In some embodiments, the alcohol dehydrogenase is co-expressed with another branched chain amino acid deaminase enzyme, e.g. a BCAA dehydrogenase, such as leucine dehydrogenase, a BCAA aminotransferase, e.g., ilvE, and/or amino acid oxidase, e.g., L-AAD, and is co-expressed with one or more keto-acid decarboxylase enzyme(s), e.g., kivD. In some embodiments, the alcohol dehydrogenase is co-expressed with another branched chain amino acid deaminase enzyme, e.g. a BCAA dehydrogenase, such as leucine dehydrogenase, a BCAA aminotransferase, e.g., ilvE, and/or amino acid oxidase, e.g., L-AAD, and is co-expressed with one or more other alcohol dehydrogenases. In some embodiments, the alcohol dehydrogenase is co-expressed with another branched chain amino acid deaminase enzyme, e.g. a BCAA dehydrogenase, such as leucine dehydrogenase, a BCAA aminotransferase, e.g., ilvE, and/or amino acid oxidase, e.g., L-AAD, and is co-expressed with one or more aldehyde dehydrogenases, e.g., padA. In some embodiments, the alcohol dehydrogenase is co-expressed with another branched chain amino acid deaminase enzyme, e.g. a BCAA dehydrogenase, such as leucine dehydrogenase, a BCAA aminotransferase, e.g., ilvE, and/or amino acid oxidase, e.g., L-AAD, is co-expressed with one or more keto-acid decarboxylase enzyme(s), e.g., kivD, and co-expressed with one or more other alcohol dehydrogenases, and/or one or more aldehyde dehydrogenases, e.g., padA. In some embodiments, the at least one alcohol dehydrogenase enzyme is coexpressed with one or more BCAA transporter(s), for example, a high affinity leucine transporter, e.g., LivKHMGF and/or low affinity BCAA transporter BrnQ. In some embodiments, the at least one alcohol dehydrogenase enzyme is coexpressed with one or more BCAA binding protein(s), for example, LivJ. In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one alcohol dehydrogenase enzyme and genetic modification that reduces export of a branched chain amino acid, e.g., a genetic mutation in a leuE gene or promoter thereof. In one embodiment, the engineered bacteria comprise gene sequence(s) encoding at least one alcohol dehydrogenase enzyme and a genetic modification that reduces or eliminates branched chain amino acid synthesis, e.g., a genetic mutation in a ilvC gene or promoter thereof.

In some embodiments, the engineered bacteria comprise gene sequence(s) encoding at least one alcohol dehydrogenase enzyme and further comprise gene sequence encoding one or more polypeptides selected from other branched chain amino acid catabolism enzyme(s), BCAA transporter(s), and BCAA binding protein(s). In some embodiments, the engineered bacteria comprise gene sequence(s) encoding at least one alcohol dehydrogenase and gene sequence(s) encoding one or more branched chain amino acid dehydrogenase(s) (e.g., leuDH). In some embodiments, the engineered bacteria comprise gene sequence(s) encoding at least one alcohol dehydrogenase and gene sequence(s) encoding one or more amino acid oxidase(s), e.g., L-AAD. In some embodiments, the engineered bacteria comprise gene sequence(s) encoding at least one alcohol dehydrogenase and gene sequence(s) encoding one or more BCAA aminotransferase(s), e.g., ilvE. In some embodiments, the engineered bacteria comprise gene sequence(s) encoding at least one alcohol dehydrogenase and gene sequence(s) encoding one or more keto-acid decarboxylase enzyme(s), e.g., kivD. In some embodiments, the engineered bacteria comprise gene sequence(s) encoding at least one alcohol dehydrogenase and gene sequence(s) encoding one or more aldehyde dehydrogenase(s) (e.g., padA). In some embodiments, the engineered bacteria comprise gene sequence(s) encoding at least one alcohol dehydrogenase and gene sequence(s) encoding one or more other alcohol dehydrogenase(s) (e.g., adh2, yqhD).

In some embodiments, the engineered bacteria comprise gene sequence(s) encoding at least one alcohol dehydrogenase and gene sequence(s) encoding one or more other branched chain amino acid catabolism enzyme(s), for example, branched chain amino acid dehydrogenase(s), such as leuDH, branched chain amino acid aminotransferase enzyme, e.g., ilvE, and/or amino acid oxidase(s), e.g. L-AAD, and gene sequence(s) encoding one or more keto-acid decarboxylase enzyme(s), e.g., kivD. In some embodiments, the engineered bacteria comprise gene sequence(s) encoding at least one alcohol dehydrogenase and gene sequence(s) encoding one or more other branched chain amino acid catabolism enzyme(s), for example, branched chain amino acid dehydrogenase(s), such as leuDH, branched chain amino acid aminotransferase enzyme, e.g., ilvE, and/or amino acid oxidase(s), e.g., L-AAD, gene sequence(s) encoding one or more keto-acid decarboxylase enzyme(s), e.g., kivD, and gene sequence(s) encoding one or more aldehyde dehydrogenase(s) (e.g., padA) and/or gene sequence(s) encoding one or more other alcohol dehydrogenase(s).

In some embodiments, the engineered bacteria comprise gene sequence(s) encoding at least one alcohol dehydrogenase and gene sequence(s) encoding one or more BCAA transporter(s) (e.g., LivKHMGF, brnQ). In some embodiments, the engineered bacteria comprise gene sequence(s) encoding at least one alcohol dehydrogenase and gene sequence(s) encoding one or more BCAA binding protein(s) (e.g., livJ). In some embodiments, the engineered bacteria comprise gene sequence(s) encoding at least alcohol dehydrogenase and gene sequence(s) encoding one or more BCAA transporter(s) (e.g., LivKHMGF, brnQ), and gene sequence(s) encoding one or more BCAA binding protein(s) (e.g., livJ). In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one alcohol dehydrogenase and genetic modification that reduces export of a branched chain amino acid, e.g., a genetic mutation in a leuE gene or promoter thereof. In one embodiment, the engineered bacteria comprise gene sequence(s) encoding at least one alcohol dehydrogenase and a genetic modification that reduces or eliminates branched chain amino acid synthesis, e.g., a genetic mutation in a ilvC gene or promoter thereof.

In one embodiment, the at least one gene encoding the branched chain amino acid alcohol dehydrogenase comprises the adh2 gene. In one embodiment, the adh2 gene has at least about 80% identity with the sequence of SEQ ID NO:38. In one embodiment, the adh2 gene has at least about 90% identity with the sequence of SEQ ID NO:38. In one embodiment, the adh2 gene has at least about 95% identity with the sequence of SEQ ID NO:38. In one embodiment, the adh2 gene has at least about 85%, 86%, 87%, 88%, 89%, 90%, 91%, 92%, 93%, 94%, 95%, 96%, 97%, 98%, or 99% identity with the sequence of SEQ ID NO:38. In another embodiment, the adh2 gene comprises the sequence of SEQ ID NO:38. In yet another embodiment, the adh2 gene consists of the sequence of SEQ ID NO:38.

In one embodiment, the at least one gene encoding the branched chain amino acid alcohol dehydrogenase comprises the adh6 gene. In one embodiment, the adh6 gene has at least about 80% identity with the sequence of SEQ ID NO:41. In one embodiment, the adh6 gene has at least about 90% identity with the sequence of SEQ ID NO:41. In one embodiment, the adh6 gene has at least about 95% identity with the sequence of SEQ ID NO:41. In one embodiment, the adh6 gene has at least about 85%, 86%, 87%, 88%, 89%, 90%, 91%, 92%, 93%, 94%, 95%, 96%, 97%, 98%, or 99% identity with the sequence of SEQ ID NO:41. In another embodiment, the adh6 gene comprises the sequence of SEQ ID NO:41. In yet another embodiment, the adh6 gene consists of the sequence of SEQ ID NO:41.

In one embodiment, the at least one gene encoding the branched chain amino acid alcohol dehydrogenase comprises the adh1 gene. In one embodiment, the adh1 gene has at least about 80% identity with the sequence of SEQ ID NO:43. In one embodiment, the adh1 gene has at least about 90% identity with the sequence of SEQ ID NO:43. In one embodiment, the adh1 gene has at least about 95% identity with the sequence of SEQ ID NO:43. In one embodiment, the adh1 gene has at least about 85%, 86%, 87%, 88%, 89%, 90%, 91%, 92%, 93%, 94%, 95%, 96%, 97%, 98%, or 99% identity with the sequence of SEQ ID NO:43. In another embodiment, the adh1 gene comprises the sequence of SEQ ID NO:43. In yet another embodiment, the adh1 gene consists of the sequence of SEQ ID NO:43.

In one embodiment, the at least one gene encoding the branched chain amino acid alcohol dehydrogenase comprises the adh3 gene. In one embodiment, the adh3 gene has at least about 80% identity with the sequence of SEQ ID NO:45. In one embodiment, the adh3 gene has at least about 90% identity with the sequence of SEQ ID NO:45. In one embodiment, the adh3 gene has at least about 95% identity with the sequence of SEQ ID NO:45. In one embodiment, the adh3 gene has at least about 85%, 86%, 87%, 88%, 89%, 90%, 91%, 92%, 93%, 94%, 95%, 96%, 97%, 98%, or 99% identity with the sequence of SEQ ID NO:45. In another embodiment, the adh3 gene comprises the sequence of SEQ ID NO:45. In yet another embodiment, the adh3 gene consists of the sequence of SEQ ID NO:45.

In one embodiment, the at least one gene encoding the branched chain amino acid alcohol dehydrogenase comprises the adh4 gene. In one embodiment, the adh4 gene has at least about 80% identity with the sequence of SEQ ID NO:47. In one embodiment, the adh4 gene has at least about 90% identity with the sequence of SEQ ID NO:47. In one embodiment, the adh4 gene has at least about 95% identity with the sequence of SEQ ID NO:47. In one embodiment, the adh4 gene has at least about 85%, 86%, 87%, 88%, 89%, 90%, 91%, 92%, 93%, 94%, 95%, 96%, 97%, 98%, or 99% identity with the sequence of SEQ ID NO:47. In another embodiment, the adh4 gene comprises the sequence of SEQ ID NO:47. In yet another embodiment, the adh4 gene consists of the sequence of SEQ ID NO:47.

In one embodiment, the at least one gene encoding the branched chain amino acid alcohol dehydrogenase comprises the adh5 gene. In one embodiment, the adh5 gene has at least about 80% identity with the sequence of SEQ ID NO:49. In one embodiment, the adh5 gene has at least about 90% identity with the sequence of SEQ ID NO:49. In one embodiment, the adh5 gene has at least about 95% identity with the sequence of SEQ ID NO:49. In one embodiment, the adh5 gene has at least about 85%, 86%, 87%, 88%, 89%, 90%, 91%, 92%, 93%, 94%, 95%, 96%, 97%, 98%, or 99% identity with the sequence of SEQ ID NO:49. In another embodiment, the adh5 gene comprises the sequence of SEQ ID NO:49. In yet another embodiment, the adh5 gene consists of the sequence of SEQ ID NO:49.

In one embodiment, the at least one gene encoding the branched chain amino acid alcohol dehydrogenase comprises the adh7 gene. In one embodiment, the adh7 gene has at least about 80% identity with the sequence of SEQ ID NO:51. In one embodiment, the adh7 gene has at least about 90% identity with the sequence of SEQ ID NO:51. In one embodiment, the adh7 gene has at least about 95% identity with the sequence of SEQ ID NO:51. In one embodiment, the adh7 gene has at least about 85%, 86%, 87%, 88%, 89%, 90%, 91%, 92%, 93%, 94%, 95%, 96%, 97%, 98%, or 99% identity with the sequence of SEQ ID NO:51. In another embodiment, the adh7 gene comprises the sequence of SEQ ID NO:51. In yet another embodiment, the adh7 gene consists of the sequence of SEQ ID NO:51.

In one embodiment, the at least one gene encoding the branched chain amino acid alcohol dehydrogenase comprises the sfa1 gene. In one embodiment, the sfa1 gene has at least about 80% identity with the sequence of SEQ ID NO:53. In one embodiment, the sfa1 gene has at least about 90% identity with the sequence of SEQ ID NO:53. In one embodiment, the sfa1 gene has at least about 95% identity with the sequence of SEQ ID NO:53. In one embodiment, the sfa1 gene has at least about 85%, 86%, 87%, 88%, 89%, 90%, 91%, 92%, 93%, 94%, 95%, 96%, 97%, 98%, or 99% identity with the sequence of SEQ ID NO:53. In another embodiment, the sfa1 gene comprises the sequence of SEQ ID NO:53. In yet another embodiment, the sfa1 gene consists of the sequence of SEQ ID NO:53.

In one embodiment, the at least one gene encoding the branched chain amino acid alcohol dehydrogenase comprises the YqhD gene. In one embodiment, the YqhD gene has at least about 80% identity with the sequence of SEQ ID NO: 60. In one embodiment, the YqhD gene has at least about 90% identity with the sequence of SEQ ID NO: 60. In one embodiment, the YghD gene has at least about 95% identity with the sequence of SEQ ID NO: 60. In one embodiment, the sfa1 gene has at least about 85%, 86%, 87%, 88%, 89%, 90%, 91%, 92%, 93%, 94%, 95%, 96%, 97%, 98%, or 99% identity with the sequence of SEQ ID NO:53. In another embodiment, the YqhD gene comprises the sequence of SEQ ID NO:53. In yet another embodiment, the YqhD gene consists of the sequence of SEQ ID NO:53.

In any of these embodiments, the alcohol dehydrogenase is coexpressed with one or more branched chain amino acid catabolism enzymes, e.g., leuDH, ilvE, L-AAD, and/or kivD, each of which are described in more detail herein. In other embodiments, the alcohol dehydrogenase is further coexpressed with a transporter of a branched chain amino acid and/or a binding protein of a BCAA.

In some embodiments, the gene sequence(s) encoding the one or more alcohol dehydrogenase is expressed under the control of a constitutive promoter. In some embodiments, the gene sequence(s) encoding the one or more b alcohol dehydrogenase is expressed under the control of an inducible promoter. In some embodiments, the gene sequence(s) encoding the one or more alcohol dehydrogenase is expressed under the control of a promoter that is directly or indirectly induced by exogenous environmental conditions. In some embodiments, the gene sequence(s) encoding the one or more alcohol dehydrogenase is expressed under the control of a promoter that is directly or indirectly induced by low-oxygen or anaerobic conditions, wherein expression of the gene encoding the alcohol dehydrogenase is activated under low-oxygen or anaerobic environments, such as the environment of the mammalian gut. In some embodiments, the gene sequence(s) encoding the one or more alcohol dehydrogenase is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

In one embodiment, wherein a branched chain amino acid catabolism enzyme is used to convert a ketoacid to its corresponding aldehyde, the recombinant bacterial cells of the invention may further comprise an aldehyde dehydrogenase enzyme in order to convert the branched chain amino acid-derived aldehyde to its respective carboxylic acid. In some embodiments, the engineered bacteria comprise gene sequence(s) encoding one or more copies of an aldehyde dehydrogenase. In some embodiments, the engineered bacteria comprise gene sequence(s) encoding one, two, three, four, five, six, or more copies of an aldehyde dehydrogenase. The one or more copies of an aldehyde dehydrogenase can be one or more copies of the same gene or can be different genes encoding aldehyde dehydrogenase, e.g., gene(s) from a different species or otherwise having a different gene sequence. The one or more copies of aldehyde dehydrogenase can be present in the bacterial chromosome or can be present in one or more plasmids. As used herein, “aldehyde dehydrogenase” refers to any polypeptide having enzymatic activity that catalyzes the conversion of a branched chain amino acid-derived aldehyde, e.g., isovaleraldehyde, isobutyraldehyde, 2-methylbutyraldehydeinto its respective carboxylic acid, e.g., isovalerate, isobutyrate, 2-methylbutyrate.

In some embodiments, the aldehyde dehydrogenase is encoded by at least one gene derived from a bacterial species. In some embodiments, the aldehyde dehydrogenase is encoded by at least one gene derived from a non-bacterial species. In some embodiments, the aldehyde dehydrogenase is encoded by at least one gene derived from a eukaryotic species, e.g., a yeast species or a plant species. In another embodiment, the aldehyde dehydrogenase is encoded by at least one gene derived from a mammalian species, e.g., human.

In one embodiment, the at least one gene encoding the aldehyde dehydrogenase is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Proteus, Pseudomonas, Psychrobacter, Ralstonia, Saccharomyces, Salmonella, Sarcina, Serratia, Staphylococcus, Streptococcus, Escherichia coli and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Enterococcus faecalis, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartita, Nasonia vitipennis, Nostoc punctiforme, Pantoea ananatis, Pantoea agglomerans, Pectobacterium atrosepticum, Pectobacterium carotovorum, Pseudomonas putida, Pseudomonas aeruginosa, Psychrobacter anticus, Proteus vulgaris, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Streptococcus faecalis, Yersinia enterocolitica, Yersinia mollaretii, Yersinia kristensenii, Yersinia rohdei, and Yersinia aldovae. In some embodiments, the aldehyde dehydrogenase is encoded by at least one gene derived from Saccharomyces cerevisiae. In another embodiment, the aldehyde dehydrogenase is encoded by at least one gene derived from E. coli. In another embodiment, the aldehyde dehydrogenase is encoded by at least one gene derived from E. Coli K-12.

In one embodiment the at least one gene encoding the branched chain amino acid dehydrogenase has been codon-optimized for use in the recombinant bacterial cell. In one embodiment, the at least one gene encoding the branched chain amino acid dehydrogenase has been codon-optimized for use in Escherichia coli.

When an aldehyde dehydrogenase is expressed in the recombinant bacterial cells disclosed herein, the bacterial cells convert more branched chain amino acid-derived aldehydes to their respective carboxylic acids than unmodified bacteria of the same bacterial subtype under the same conditions (e.g., culture or environmental conditions). Thus, the genetically engineered bacteria comprising at least one heterologous gene encoding an aldehyde dehydrogenase may be used to catabolize excess branched chain amino acid-derived aldehydes, e.g., isovaleraldehyde, to treat a disease associated with a branched chain amino acid, including Maple Syrup Urine Disease (MSUD). In some embodiments, the aldehyde dehydrogenase is co-expressed with one or more branched chain amino acid catabolism enzymes, e.g., leuDH, ilvE, L-AAD, and/or kivD.

The present disclosure encompasses genes encoding an aldehyde dehydrogenase comprising amino acids in its sequence that are substantially the same as an amino acid sequence described herein. The present disclosure further comprises genes encoding functional fragments of an aldehyde dehydrogenase or functional variants of an aldehyde dehydrogenase. As used herein, the term “functional fragment thereof” or “functional variant thereof” of an aldehyde dehydrogenase gene relates to a sequence having qualitative biological activity in common with the wild-type aldehyde dehydrogenase, from which the fragment or variant was derived. For example, a functional fragment or a functional variant of a mutated aldehyde dehydrogenase is one which retains essentially the same ability to catabolize BCAAs as aldehyde dehydrogenase from which the functional fragment or functional variant was derived. For example, a polypeptide having aldehyde dehydrogenase activity may be truncated at the N-terminus or C-terminus and the retention aldehyde dehydrogenase activity assessed using assays known to those of skill in the art, including the exemplary assays provided herein. In one embodiment, the recombinant bacterial cell disclosed herein comprises a heterologous gene encoding an an aldehyde dehydrogenase functional variant. In another embodiment, the recombinant bacterial cell disclosed herein comprises a heterologous gene encoding alcohol dehydrogenase functional fragment.

In some embodiments, the at least one gene encoding an aldehyde dehydrogenase is mutagenized, mutants exhibiting increased activity are selected, and the mutagenized gene(s) encoding the aldehyde dehydrogenase are isolated and inserted into the bacterial cell. In some embodiments, the at least one gene encoding the aldehyde dehydrogenase is mutagenized, mutants exhibiting decreased activity are selected, and the mutagenized gene(s) encoding the aldehyde dehydrogenase are isolated and inserted into the bacterial cell. The gene comprising the modifications described herein may be present on a plasmid or chromosome.

Assays for testing the activity of an aldehyde dehydrogenase, an aldehyde dehydrogenase functional variant, or an aldehyde dehydrogenase functional fragment are well known to one of ordinary skill in the art. For example, aldehyde dehydrogenase activity can be assessed by expressing the protein, functional variant, or fragment thereof, in a recombinant bacterial cell that lacks endogenous aldehyde dehydrogenase activity. Also, activity can be assessed using the enzymatic assay methods as described by Kagi et al. (J. Biol. Chem., 235:3188-92, 1960), and Walker (Biochem. Educ., 20(1):42-43, 1992).

In some embodiments, the aldehyde dehydrogenase is co-expressed with an additional branched chain amino acid catabolism enzyme. In some embodiments, the aldehyde dehydrogenase is co-expressed with one or more additional branched chain amino acid catabolism enzyme(s) selected from a branched chain amino acid deaminase enzyme, e.g. a BCAA dehydrogenase, such as leucine dehydrogenase; a BCAA aminotransferase, e.g., ilvE, and a branched chain amino acid oxidase, e.g., L-AAD. In some embodiments, the aldehyde dehydrogenase is co-expressed with one or more keto-acid decarboxylase enzyme(s), e.g., kivD. In some embodiments, the aldehyde dehydrogenase is co-expressed with one or more alcohol dehydrogenases. In some embodiments, the branched chain amino acid oxidase is co-expressed with one or more other aldehyde dehydrogenases. In some embodiments, the aldehyde dehydrogenase is co-expressed with one or more keto-acid decarboxylase enzymes, e.g., kivD and one or more other aldehyde dehydrogenase enzymes. In some embodiments, the aldehyde dehydrogenase is co-expressed with one or more keto-acid decarboxylase enzymes, e.g., kivD and one or more alcohol dehydrogenase enzymes. In some embodiments, the aldehyde dehydrogenase is co-expressed with one or more keto-acid decarboxylase enzymes, e.g., kivD, one or more other aldehyde dehydrogenase enzymes and one or more alcohol dehydrogenase enzymes.

In some embodiments, the aldehyde dehydrogenase is co-expressed with another branched chain amino acid deaminase enzyme, e.g. a BCAA dehydrogenase, such as leucine dehydrogenase, a BCAA aminotransferase, e.g., ilvE, and/or amino acid oxidase, e.g., L-AAD, and is co-expressed with one or more keto-acid decarboxylase enzyme(s), e.g., kivD. In some embodiments, the aldehyde dehydrogenase is co-expressed with another branched chain amino acid deaminase enzyme, e.g. a BCAA dehydrogenase, such as leucine dehydrogenase, a BCAA aminotransferase, e.g., ilvE, and/or amino acid oxidase, e.g., L-AAD, and is co-expressed with one or more alcohol dehydrogenases. In some embodiments, the aldehyde dehydrogenase is co-expressed with another branched chain amino acid deaminase enzyme, e.g. a BCAA dehydrogenase, such as leucine dehydrogenase, a BCAA aminotransferase, e.g., ilvE, and/or amino acid oxidase, e.g., L-AAD, and is co-expressed with one or more other aldehyde dehydrogenases. In some embodiments, the aldehyde dehydrogenase is co-expressed with another branched chain amino acid deaminase enzyme, e.g. a BCAA dehydrogenase, such as leucine dehydrogenase, a BCAA aminotransferase, e.g., ilvE, and/or amino acid oxidase, e.g., L-AAD, is co-expressed with one or more keto-acid decarboxylase enzyme(s), e.g., kivD, and co-expressed with one or more alcohol dehydrogenases, and/or one or more other aldehyde dehydrogenases. In some embodiments, the at least one aldehyde dehydrogenase enzyme is coexpressed with one or more BCAA transporter(s), for example, a high affinity leucine transporter, e.g., LivKHMGF and/or low affinity BCAA transporter BrnQ. In some embodiments, the at least one aldehyde dehydrogenase enzyme is coexpressed with one or more BCAA binding protein(s), for example, LivJ. In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one aldehyde dehydrogenase enzyme and genetic modification that reduces export of a branched chain amino acid, e.g., a genetic mutation in a leuE gene or promoter thereof. In one embodiment, the engineered bacteria comprise gene sequence(s) encoding at least one aldehyde dehydrogenase enzyme and a genetic modification that reduces or eliminates branched chain amino acid synthesis, e.g., a genetic mutation in a ilvC gene or promoter thereof.

In some embodiments, the engineered bacteria comprise gene sequence(s) encoding at least one aldehyde dehydrogenase enzyme and further comprise gene sequence encoding one or more polypeptides selected from other branched chain amino acid catabolism enzyme(s), BCAA transporter(s), and BCAA binding protein(s). In some embodiments, the engineered bacteria comprise gene sequence(s) encoding at least one aldehyde dehydrogenase and gene sequence(s) encoding one or more branched chain amino acid dehydrogenase(s) (e.g., leuDH). In some embodiments, the engineered bacteria comprise gene sequence(s) encoding at least one aldehyde dehydrogenase and gene sequence(s) encoding one or more amino acid oxidase(s), e.g., L-AAD. In some embodiments, the engineered bacteria comprise gene sequence(s) encoding at least one aldehyde dehydrogenase and gene sequence(s) encoding one or more BCAA aminotransferase(s), e.g., ilvE. In some embodiments, the engineered bacteria comprise gene sequence(s) encoding at least one aldehyde dehydrogenase and gene sequence(s) encoding one or more keto-acid decarboxylase enzyme(s), e.g., kivD. In some embodiments, the engineered bacteria comprise gene sequence(s) encoding at least one aldehyde dehydrogenase and gene sequence(s) encoding one or more other aldehyde dehydrogenase(s). In some embodiments, the engineered bacteria comprise gene sequence(s) encoding at least one aldehyde dehydrogenase and gene sequence(s) encoding one or more alcohol dehydrogenase(s).

In some embodiments, the engineered bacteria comprise gene sequence(s) encoding at least one aldehyde dehydrogenase and gene sequence(s) encoding one or more other branched chain amino acid catabolism enzyme(s), for example, branched chain amino acid dehydrogenase(s), such as leuDH, branched chain amino acid aminotransferase enzyme, e.g., ilvE, and/or amino acid oxidase(s), e.g. L-AAD, and gene sequence(s) encoding one or more keto-acid decarboxylase enzyme(s), e.g., kivD. In some embodiments, the engineered bacteria comprise gene sequence(s) encoding at least one aldehyde dehydrogenase and gene sequence(s) encoding one or more other branched chain amino acid catabolism enzyme(s), for example, branched chain amino acid dehydrogenase(s), such as leuDH, branched chain amino acid aminotransferase enzyme, e.g., ilvE, and/or amino acid oxidase(s), e.g., L-AAD, gene sequence(s) encoding one or more keto-acid decarboxylase enzyme(s), e.g., kivD, and gene sequence(s) encoding one or more other aldehyde dehydrogenase(s) and/or gene sequence(s) encoding one or more alcohol dehydrogenase(s).

In some embodiments, the engineered bacteria comprise gene sequence(s) encoding at least one aldehyde dehydrogenase and gene sequence(s) encoding one or more BCAA transporter(s) (e.g., LivKHMGF, brnQ). In some embodiments, the engineered bacteria comprise gene sequence(s) encoding at least one aldehyde dehydrogenase and gene sequence(s) encoding one or more BCAA binding protein(s) (e.g., livJ). In some embodiments, the engineered bacteria comprise gene sequence(s) encoding at least one aldehyde dehydrogenase and gene sequence(s) encoding one or more BCAA transporter(s) (e.g., LivKHMGF, brnQ), and gene sequence(s) encoding one or more BCAA binding protein(s) (e.g., livJ). In one specific embodiment, the gene sequence encoding LivKHMGF is SEQ ID NO: 91. In another specific embodiment, the gene sequence encoding brnQ is SEQ ID NO: 64. In another specific embodiment, the gene sequence encoding livJ is SEQ ID NO: 12.

In some embodiments, the engineered bacteria comprise gene sequence(s) encoding at least one aldehyde dehydrogenase and genetic modification that reduces export of a branched chain amino acid, e.g., a genetic mutation in a leuE gene or promoter thereof. In one embodiment, the engineered bacteria comprise gene sequence(s) encoding at least one aldehyde dehydrogenase and a genetic modification that reduces or eliminates branched chain amino acid synthesis, e.g., a genetic mutation in a ilvC gene or promoter thereof.

In some embodiments, the gene sequence(s) encoding the one or more aldehyde dehydrogenase is expressed under the control of a constitutive promoter. In some embodiments, the gene sequence(s) encoding the one or more aldehyde dehydrogenase is expressed under the control of an inducible promoter. In some embodiments, the gene sequence(s) encoding the one or more aldehyde dehydrogenase is expressed under the control of a promoter that is directly or indirectly induced by exogenous environmental conditions. In some embodiments, the gene sequence(s) encoding the one or more aldehyde dehydrogenase is expressed under the control of a promoter that is directly or indirectly induced by low-oxygen or anaerobic conditions, wherein expression of the gene encoding the aldehyde dehydrogenase is activated under low-oxygen or anaerobic environments, such as the environment of the mammalian gut. In some embodiments, the gene sequence(s) encoding the one or more aldehyde dehydrogenase is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{azaC} promoter, a P_{araBAD} promoter, and a P_{TetR} promoter, each of which are described in more detail herein.

In one embodiment, the at least one gene encoding the branched chain amino acid aldehyde dehydrogenase comprises the PadA gene. In one embodiment, the PadA gene has at least about 80% identity with the sequence of SEQ ID NO: 62. In one embodiment, the PadA gene has at least about 90% identity with the sequence of SEQ ID NO: 62. In one embodiment, the PadA gene has at least about 95% identity with the sequence of SEQ ID NO: 62. In one embodiment, the PadA gene has at least about 85%, 86%, 87%, 88%, 89%, 90%, 91%, 92%, 93%, 94%, 95%, 96%, 97%, 98%, or 99% identity with the sequence of SEQ ID NO: 62. In another embodiment, the PadA gene comprises the sequence of SEQ ID NO: 62. In yet another embodiment, the PadA gene consists of the sequence of SEQ ID NO: 62.

In some embodiments, the aldehyde dehydrogenase, e.g., padA, is coexpressed with one or more branched chain amino acid catabolism enzymes, e.g., leuDH, ilvE, L-AAD, or kivD, each of which are described in more detail herein. In another embodiment, the aldehyde dehydrogenase is further coexpressed with a transporter of a branched chain amino acid.

E. LIU Operon

In one embodiment, the branched chain amino acid catabolism enzyme comprises the enzymes expressed by the Liu operon. LiuA is a terpenoid-specific, FAD-dependent acyl-CoA dehydrogenase that catalyzes the formation of a carbon-carbon double bond in methyl-branched substrates, similar to citronellyl-CoA dehydrogenase. The gene encoding this enzyme is part of the L-leucine and isovalerate utilizing liuABCDE gene cluster in this organism. A liuA insertion mutant was unable to utilize acyclic terpenes, L-leucine or isovalerate, but could utilize succinate (ForsterFromme and Jendrossek; Biochemical characterization of isovaleryl-CoA dehydrogenase (LiuA) of Pseudomonas aeruginosa and the importance of liu genes fora functional catabolic pathway of methyl-branched compounds. FEMS Microbiol Lett. 2008 September; 286(1):78-84.] (see pathways citronellol degradation, cis-genanyl-CoA degradation and L-leucine degradation I).

In some embodiments, wherein a branched chain amino acid catabolism enzyme is a branched chain keto acid dehydrogenase is used to oxidatively decarboxylate all three branched chain keto acids (α-ketoisocaproate, α-keto-β-methylvalerate, and α-ketoisovalerate) into their respective acyl-CoA derivatives, (isovaleryl-CoA, α-methylbutyryl-CoA, isobutyryl-CoA), the recombinant bacterial cells may further comprise an Liu operon, which can convert isovaleryl-CoA to acetoacetate and acetylCoA. In some embodiments, a Liu operon circuit is useful in combination with a Bkd complex, e.g., in the treatment of MSUD. Alternatively, in some embodiments, the bacterial cells comprise a Liu operon circuit, e.g., in the absence of a Bkd complex. In some embodiments, the Liu operon is useful in the absence of the Bkd complex, e.g., in the treatment of isovaleric academia.

Isovaleric academia ((OMIM) 243500), also called isovaleric aciduria or isovaleric acid CoA dehydrogenase deficiency is a rare autosomal recessive (Lee et al., Different spectrum of mutations of isovaleryl-CoA dehydrogenase (IVD) gene in Korean patients with isovaleric academia; Mol Genet Metab. 2007 September-October; 92(0): 71-77) metabolic disorder which disrupts or prevents normal metabolism of the branched-chain amino acid leucine (Dionisi-Vici et al., J Inherit Metab Dis. 2006 April-June; 29(2-3):383-9. In isovaleric academia patients, a mutation in the gene encoding IVD (isovaleric acid-CoA dehydrogenase; EC 1.3.8.4). blocks the third step in leucine degradation and leads to toxic levels of isovalerate, damaging the brain and nervous system. In some embodiments of the disclosure, the amount of isovaleric acid using a synthetic probiotic strain is reduced, e.g. for the treatment of isovaleric acidemia. In one embodiments, leucine and its ketoacid alpha-ketoisocaproate, are degraded similar to the strategy used to treat MSUD. In another embodiment, isovalerate is first converted to isovaleryl-CoA by expressing an acyl-CoA synthetase with activity against isovalerate (isovaleryl-CoA synthetase), then metabolized isovaleryl-CoA to acetoacetate and acetyl-CoA by expressing four additional enzymes: an isovaleryl-CoA dehydrogenase, a 3-methylcrotonyl-CoA carboxylase, a 3-methylglutaconyl-CoA hydratase and an hydroxymethylglutaryl-CoA lyase. In one embodiment, a Liu Operon is used. In one embodiment, the genetically engineered bacteria express an acyl CoA synthetase, which can convert isovalerate to isovaleryl-CoA. In one embodiment, the acyl CoA synthetase is Lbul. In one embodiment, a acyl-CoA synthetase is used in the treatment of isovaleric academia.

‘Classical’ organic acidurias, propionic aciduria, methylmalonic aciduria and isovaleric aciduria: long-term outcome and effects of expanded newborn screening using tandem mass spectrometry). It is a classical type of organic acidemia.

Transporter (Importer) of a Branched Chain Amino Acid

In some embodiments, a recombinant bacterial cell disclosed herein comprising gene sequence(s) encoding at least one branched chain amino acid catabolism enzyme (e.g., in some embodiments expressed on a high-copy plasmid) does not increase branched chain amino acid catabolism or decrease branched chain amino acid levels in the absence of a heterologous transporter (importer) of the branched chain amino acid, e.g., leucine, and additional copies of a native importer of the branched chain amino acid, e.g., livKHMGF. It has been surprisingly discovered that in some embodiments, the rate-limiting step of branched chain amino acid catabolism, e.g., leucine catabolism, is not expression of a branched chain amino acid catabolism enzyme, but rather availability of the branched chain amino acid, e.g., leucine (see, e.g., FIG. 18). Thus, in some embodiments, it may be advantageous to increase branched chain amino acid transport, e.g., leucine transport, into the cell, thereby enhancing branched chain amino acid catabolism. Surprisingly, in conjunction with overexpression of a transporter of a branched chain amino acid, e.g., LivKHMGF, even low copy number plasmids comprising a gene encoding at least one branched chain amino acid catabolism enzyme are capable of almost completely eliminating a branched chain amino acid, e.g., leucine, from a sample (see, e.g., FIG. 18). Furthermore, in some embodiments that incorporate a transporter of a branched chain amino acid into the recombinant bacterial cell, there may be additional advantages to using a low-copy plasmid comprising the gene encoding the branched chain amino acid catabolism enzyme in conjunction in order to enhance the stability of expression of the branched chain amino acid catabolism enzyme, while maintaining high branched chain amino acid catabolism and to reduce negative selection pressure on the transformed bacterium. In alternate embodiments, the branched chain amino acid transporter is used in conjunction with a high-copy plasmid. In alternate embodiments, the gene(s) at least one BCAA catabolism enzyme is integrated in the bacterial chromosome.

In some embodiments, in which the engineered bacterial cell comprises gene sequence encoding a branched amino acid transporter, the bacterial cell comprises gene sequence encoding one branched chain amino acid catabolism enzyme. In other embodiments, in which the engineered bacterial cell comprises gene sequence encoding a branched amino acid transporter, the bacterial cell comprises gene sequence(s) encoding two branched chain amino acid catabolism enzymes. In other embodiments, in which the engineered bacterial cell comprises gene sequence encoding a branched amino acid transporter, the bacterial cell comprises gene sequence(s) encoding three or more branched chain amino acid catabolism enzymes. In other embodiments, in which the engineered bacterial cell comprises gene sequence encoding a branched amino acid transporter, the bacterial cell comprises gene sequence(s) encoding four, five, six or more branched chain amino acid catabolism enzymes.

In some embodiments, the branched chain amino acid catabolism enzyme converts a branched chain amino acid, e.g., leucine, valine, isoleucine, into its corresponding branched chain alpha-keto acid counterpart. In other embodiments, the branched chain amino acid catabolism enzyme converts a branched chain alpha-keto acid, e.g., alpha-ketoisocaproate, alpha-keto-beta-methylvalerate, alpha-ketoisovalerate into its corresponding aldehyde. In other embodiments, the branched chain amino acid catabolism enzyme converts a branched chain alpha-keto acid, e.g., alpha-ketoisocaproate, alpha keto-beta-methylvalerate, alpha-ketoisovalerate into its corresponding acetyl-CoA, e.g., isovaleryl-CoA, alpha-methylbutyryl-CoA, isobutyl-CoA. In other embodiments, the branched chain amino acid catabolism enzyme converts a branched chain aldehyde to its corresponding alcohol. In another embodiment, the branched chain amino acid catabolism enzyme converts a branched chain aldehyde to its corresponding carboxylic acid.

In some embodiments, the engineered bacteria comprising gene sequence(s) encoding one or more BCAA transporter(s), further comprise gene sequence(s) encoding one or more BCAA catabolism enzymes selected from BCAA dehydrogenase, e.g., leuDH, BCAA amino transferase, e.g., ilvE, and amino oxidase, e.g., L-AAD. In some embodiments, the engineered bacteria comprising gene sequence(s) encoding one or more BCAA transporter(s), further comprise gene sequence(s) encoding one or more BCAA catabolism enzymes selected from BCAA dehydrogenase, e.g., leuDH, BCAA amino transferase, e.g., ilvE, and amino oxidase, e.g., L-AAD and gene sequence(s) encoding one or more branched chain keto acid dehydrogenase enzyme(s) (BCKD). In some embodiments, the engineered bacteria comprising gene sequence(s) encoding one or more BCAA transporter(s), further comprise gene sequence(s) encoding one or more BCAA catabolism enzymes selected from BCAA dehydrogenase, e.g., leuDH, BCAA amino transferase, e.g., ilvE, and amino oxidase, e.g., L-AAD and gene sequence(s) encoding one or more keto acid decarboxylase enzyme(s), e.g., kivD. In some embodiments, the engineered bacteria comprising gene sequence(s) encoding one or more BCAA transporter(s), further comprise gene sequence(s) encoding one or more BCAA catabolism enzymes selected from BCAA dehydrogenase, e.g., leuDH, BCAA amino transferase, e.g., ilvE, and amino oxidase, e.g., L-AAD and gene sequence(s) encoding one or more keto acid decarboxylase enzyme(s), e.g., kivD, and gene sequence(s) encoding one or more alcohol dehydrogenase enzyme(s) and/or gene sequence(s) encoding one or more aldehyde dehydrogenase enzyme(s).). In any of these embodiments, the engineered bacteria also comprise a genetic modification that reduces export of a branched chain amino acid, e.g., a genetic mutation in a leuE gene or promoter thereof. In any of these embodiments, the bacteria also comprise a genetic modification that reduces or eliminates branched chain amino acid synthesis, e.g., a genetic mutation in a ilvC gene or promoter thereof. In one embodiment, the bacterial cell comprises gene sequence encoding one or more transporter(s) of branched chain amino acids, gene sequence(s) encoding one or more branched chain amino acid catabolism enzyme(s), and at least one genetic modification that reduces export of a branched chain amino acid, e.g., genetic modification in a leuE gene or promoter thereof. In one embodiment, the bacterial cell comprises gene sequence encoding one or more transporter(s) of branched chain amino acids, gene sequence(s) encoding one or more branched chain amino acid catabolism enzyme(s), and at least one genetic modification that reduces or eliminates branched chain amino acid synthesis, e.g., a genetic mutation in a ilvC gene or promoter thereof. In one embodiment, the bacterial cell comprises gene sequence encoding one or more transporter(s) of branched chain amino acids, gene sequence(s) encoding one or more branched chain amino acid catabolism enzyme(s), at least one genetic modification that reduces export of a branched chain amino acid., and at least one genetic modification that reduces or eliminates branched chain amino acid synthesis. In any of these embodiments, the engineered bacterial cell may further comprise gene sequence encoding livJ, which brings BCAA into the bacterial cell.

The uptake of branched chain amino acids into bacterial cells is mediated by proteins well known to those of skill in the art. For example, two well characterized BCAA transport systems have been characterized in several bacteria, including Escherichia coli. BCAAs are transported by two systems into bacterial cells (i.e., imported), the osmotic-shock-sensitive systems designated LIV-I and LS (leucine-specific), and by an osmotic-shock resistant system, BrnQ, formerly known as LIV-II (see Adams et al., J. Biol. Chem. 265:11436-43 (1990); Anderson and Oxender, J. Bacteriol. 130:384-92 (1977); Anderson and Oxender, J. Bacteriol. 136:168-74 (1978); Haney et al., J. Bacteriol. 174:108-15 (1992); Landick and Oxender, J. Biol. Chem. 260:8257-61 (1985); Nazos et al., J. Bacteriol. 166:565-73 (1986); Nazos et al., J. Bacteriol. 163:1196-202 (1985); Oxender et al., Proc. Natl. Acad. Sci. USA 77:1412-16 (1980); Quay et al., J. Bacteriol. 129:1257-65 (1977); Rahmanian et al., J. Bacteriol. 116:1258-66 (1973); Wood, J. Biol. Chem. 250:4477-85 (1975); Guardiola et al., J. Bacteriol. 117:393-405 (1974); Guardiola and Iaccarino, J. Bacteriol. 108:1034-44 (1971); Ohnishi et al., Jpn. J. Genet. 63:343-57)(1988); Yamato and Anraku, J. Bacteriol. 144:36-44 (1980); and Yamato et al., J. Bacteriol. 138:24-32 (1979)). Transport by the BrnQ system is mediated by a single membrane protein. Transport mediated by the LIV-I system is dependent on the substrate binding protein LivJ (also known as LIV-BP), while transport mediated by LS system is mediated by the substrate binding protein LivK (also known as LS-BP). LivJ is encoded by the livJ gene, and binds isoleucine, leucine and valine with K_{d} values of ˜10⁻⁶ and ˜10⁻⁷ M, while LivK is encoded by the livK gene, and binds leucine with a K_{d} value of ˜10⁻⁶M (See Landick and Oxender, J. Biol. Chem. 260:8257-61 (1985)). Both LivJ and LivK interact with the inner membrane components LivHMGF to enable ATP-hydrolysis-coupled transport of their substrates into the cell, forming the LIV-I and LS transport systems, respectively. The LIV-I system transports leucine, isoleucine and valine, and to a lesser extent serine threonine and alanine, whereas the LS system only transports leucine. The six genes encoding the E. coli LIV-I and LS systems are organized into two transcriptional units, with livKHMGF transcribed as a single operon, and livJ transcribed separately. LivKHMGF is an ABC transporter comprised of five subunits, including LivK, which is a periplasmic amino acid binding protein, LivHM, which are memebrane subunits, and LivGF, which are ATP-binding subunits. The Escherichia coli liv genes can be grouped according to protein function, with the livJ and livK genes encoding periplasmic binding proteins with the binding affinities described above, the livH and livM genes encoding inner membrane permeases, and the livG and livF genes encoding cytoplasmic ATPases.

BrnQ is a branched chain amino acid transporter highly similar to the Salmonella typhimurium BrnQ branched chain amino acid transporter (Ohnishi et al., Cloning and nucleotide sequence of the brnQ gene, the structural gene for a membrane-associated component of the LIV-II transport system for branched-chain amino acids in Salmonella typhimurium. Jpn J Genet. 1988 August; 63(4):343-57) and corresponds to the Liv-II branched chain amino acid transport system in E. coli, which has been shown to transport leucine, valine, and isoleucine (Guardiola et al., Mutations affecting the different transport systems for isoleucine, leucine, and valine in Escherichia coli K-12. J Bacteriol. 1974 February; 117(2):393-405), Guardiola and Oxender, Genetic separation of high- and low-affinity transport systems for branched-chain amino acids in Escherichia coli K-12. J Bacteriol. 1978 October; 136(1):168-74., Anderson and Oxender, Genetic separation of high- and low-affinity transport systems for branched-chain amino acids in Escherichia coli K-12 J Bacteriol. 1978 October; 136(1):168-74.78). BrnQ is a member of the LIVCS family of branched chain amino acid transporters and likely functions as a sodium/branched chain amino acid symporter.

Branched chain amino acid transporters, e.g., leucine importers, may be expressed or modified in the bacteria disclosed herein in order to enhance branched chain amino acid, e.g., leucine, transport into the cell. For example, the gene sequence(s) for endogenous transporter(s) may be modified (e.g., codon-optimized and/or expressed by a strong promoter) to overexpress the transporter and/or additional copies of the transporter may be added. Alternatively, or additionally, gene sequence(s) for one or more non-endogenous or non-native transporters may be expressed in the bacterial cell. Specifically, when the transporter of a branched chain amino acid is expressed in the recombinant bacterial cells disclosed herein, the bacterial cells import more branched chain amino acids into the cell when the transporter is expressed than unmodified bacteria of the same bacterial subtype under the same conditions (not expressing the transporter). Thus, in some embodiments, the engineered bacteria comprise gene sequence(s) encoding one or more transporter(s) of a branched chain amino acid, e.g., leucine, which may be used to import branched chain amino acids, e.g., leucine, into the bacteria so that any gene encoding a branched chain amino acid catabolism enzyme expressed in the bacteria catabolize the branched chain amino acid, e.g., leucine, to treat diseases associated with the catabolism of branched chain amino acids, such as Maple Syrup Urine Disease (MSUD). In one embodiment, the bacterial cell comprises gene sequence(s) encoding one or more transporter(s) of branched chain amino acids. In one embodiment, the bacterial cell comprises gene sequence(s) encoding one or more transporter(s) of branched chain amino acids and a gene sequence(s) encoding one or more branched chain amino acid catabolism enzyme(s). In one embodiment, the bacterial cell comprises gene sequence(s) encoding a one or more transporter(s) of branched chain amino acids and a genetic modification that reduces export of a branched chain amino acid, e.g., a genetic mutation in a leuE gene or promoter thereof. In one embodiment, the bacterial cell comprises gene sequence(s) encoding one or more transporter(s) of branched chain amino acids and a genetic modification that reduces or eliminates branched chain amino acid synthesis, e.g., a genetic mutation in a ilvC gene or promoter thereof. In one embodiment, the bacterial cell comprises gene sequence encoding one or more transporter(s) of branched chain amino acids, gene sequence(s) encoding one or more branched chain amino acid catabolism enzyme(s), and at least one genetic modification that reduces export of a branched chain amino acid. In one embodiment, the bacterial cell comprises gene sequence encoding one or more transporter(s) of branched chain amino acids, gene sequence(s) encoding one or more branched chain amino acid catabolism enzyme(s), and at least one genetic modification that reduces or eliminates branched chain amino acid synthesis. In one embodiment, the bacterial cell comprises gene sequence encoding one or more transporter(s) of branched chain amino acids, gene sequence(s) encoding one or more branched chain amino acid catabolism enzyme(s), at least one genetic modification that reduces export of a branched chain amino acid., and at least one genetic modification that reduces or eliminates branched chain amino acid synthesis. In any of these embodiments, the engineered bacterial cell may further comprise gene sequence encoding livJ, which brings BCAA into the bacterial cell. In any of these embodiments, the transporter may be a native transporter, e.g., the bacteria may comprise additional copies of the native transporter. In any of these embodiments, the transporter may be a non-native transporter. In any of these embodiments, the transporter may be LivKHMGF. In any of these embodiments, the transporter may be brnQ. In any of these embodiments, the bacterial cell may comprise gene sequence(s) encoding LivKHMGF and brnQ.

In some embodiments, the engineered bacteria comprise gene sequence(s) encoding one or more BCAA catabolism enzyme(s) and gene sequence(s) encoding one or more BCAA transporters, in which the gene sequence(s) encoding one or more BCAA catabolism enzyme(s) and the gene sequence(s) encoding one or more transporter(s) are operably linked to different copies of the same promoter. In some embodiments, the engineered bacteria comprise gene sequence(s) encoding one or more BCAA catabolism enzyme(s) and gene sequence(s) encoding one or more BCAA transporters, in which the gene sequence(s) encoding one or more BCAA catabolism enzyme(s) and the gene sequence(s) encoding one or more transporter(s) are operably linked to different promoters. Thus, in some embodiments, the disclosure provides a bacterial cell that comprises gene sequence(s) encoding one or more branched chain amino acid catabolism enzyme(s) operably linked to a first promoter and gene sequence encoding one or more transporter(s) of a branched chain amino acid, e.g., leucine. In some embodiments, the disclosure provides a bacterial cell that comprises gene sequence(s) encoding one or more transporters of a branched chain amino acid operably linked to the first promoter. In other embodiments, the disclosure provides a bacterial cell impressing gene sequence(s) encoding one or more branched chain amino acid catabolism enzyme(s) operably linked to a first promoter and gene sequence(s) encoding one or more transporter(s) of a branched chain amino acid operably linked to a second promoter. In one embodiment, the first promoter and the second promoter are separate copies of the same promoter. In another embodiment, the first promoter and the second promoter are different promoters. In one embodiment, the first promoter and the second promoter are inducible promoters. In another embodiment, the first promoter is an inducible promoter and the second promoter is a constitutive promoter. In some embodiments, the gene sequence(s) encoding the one or more BCAA catabolism enzymes and the gene sequence(s) encoding the one or more transporters is expressed under the control of a constitutive promoter. In some embodiments, the gene sequence(s) encoding the one or more BCAA catabolism enzymes and the gene sequence(s) encoding the one or more BCAA transporters is expressed under the control of an inducible promoter. In some embodiments, the gene sequence(s) encoding the one or more BCAA transporters is expressed under the control of an inducible promoter that is directly or indirectly induced by exogenous environmental conditions. In some embodiments, the gene sequence(s) encoding the one or more BCAA catabolism enzymes and the gene sequence(s) encoding the one or more BCAA transporters is expressed under the control of an inducible promoter that is directly or indirectly induced by exogenous environmental conditions. In some embodiments, the gene sequence(s) encoding the one or more BCAA transporters is expressed under the control of an inducible promoter that is directly or indirectly induced by low-oxygen or anaerobic conditions, wherein expression of the gene encoding the one or more transporters is activated under low-oxygen or anaerobic environments, such as the environment of the mammalian gut. In some embodiments, the gene sequence(s) encoding the one or more BCAA catabolism enzymes and the gene sequence(s) encoding the one or more BCAA transporters is expressed under the control of an inducible promoter that is directly or indirectly induced by low-oxygen or anaerobic conditions, wherein expression of the gene(s) encoding the one or more BCAA catabolism enzymes and expression of the gene(s) encoding the one or more BCAA transporters is activated under low-oxygen or anaerobic environments, such as the environment of the mammalian gut. In some embodiments, the gene sequence(s) encoding the one or more BCAA transporters is expressed under the control of an inducible promoter that is directly or indirectly induced by inflammatory conditions. In some embodiments, the gene sequence(s) encoding the one or more BCAA catabolism enzymes and the gene sequence(s) encoding the one or more BCAA transporters is expressed under the control of an inducible promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein.

In one embodiment, the bacterial cell comprises at least one gene encoding a transporter of a branched chain amino acid from a different organism, e.g., a different species of bacteria. In one embodiment, the bacterial cell comprises at least one native gene encoding a transporter of a branched chain amino acid. In some embodiments, the at least one native gene encoding a transporter of a branched chain amino acid is not modified. In another embodiment, the bacterial cell comprises more than one copy of at least one native gene encoding a transporter of a branched chain amino acid. In yet another embodiment, the bacterial cell comprises a copy of at least one gene encoding a native transporter of a branched chain amino acid, as well as at least one copy of at least one heterologous gene encoding a transporter of a branched chain amino acid. The heterologous gene sequence may encode an additional copy or copies of the native transporter, may encode one or more copies of a non-native transporter, and/or may encode one or more copies of a homologous or different transporter from a different bacterial species. In one embodiment, the bacterial cell comprises at least one, two, three, four, five, or six copies of the at least one heterologous gene encoding a transporter of a branched chain amino acid. In one embodiment, the bacterial cell comprises multiple copies of the at least one heterologous gene encoding a transporter of a branched chain amino acid. In one embodiment, the bacterial cell comprises gene sequence(s) encoding two or more different transporters of a branched chain amino acid. In one embodiment, the gene sequence(s) encoding two or more different transporters of a branched chain amino acid is under the control of one or more inducible promoters. In one embodiment, the gene sequence(s) encoding two or more different transporters of a branched chain amino acid is under the control of one or more constitutive promoters. In one embodiment, the gene sequence(s) encoding two or more different transporters of a branched chain amino acid is under the control of at least one inducible promoter and at least one constitutive promoter. In any of these embodiments, the gene sequence(s) encoding the one or more BCAA transporter(s) may be present on one or more plasmids. In any of these embodiments, the gene sequence(s) encoding the one or more BCAA transporter(s) may be present in the bacterial chromosome.

In one embodiment, the transporter of a branched chain amino acid imports a branch chain amino acid into the bacterial cell. In one embodiment, the transporter of a branched chain amino acid imports leucine into the bacterial cell. In one embodiment, the transporter of a branched chain amino acid imports isoleucine into the bacterial cell. In one embodiment, the transporter of a branched chain amino acid imports valine into the bacterial cell. In one embodiment, the transporter of a branched chain amino acid imports one or more of leucine, isoleucine, and valine into the bacterial cell.

In some embodiments, the transporter of a branched chain amino acid is encoded by a transporter of a branched chain amino acid gene derived from a bacterial genus or species, including but not limited to, Bacillus, Campylobacter, Clostridium, Escherichia, Lactobacillus, Pseudomonas, Salmonella, Staphylococcus, Bacillus subtilis, Campylobacter jejuni, Clostridium perfringens, Escherichia coli, Lactobacillus delbrueckii, Pseudomonas aeruginosa, Salmonella typhimurium, or Staphylococcus aureus. In some embodiments, the bacterial species is Escherichia coli. In some embodiments, the bacterial species is Escherichia coli strain Nissle.

Multiple distinct transporters of branched chain amino acids are known in the art. In one embodiment, the at least one gene encoding a transporter of a branched chain amino acid is the brnQ gene. In one embodiment, the at least one gene encoding a transporter of a branched chain amino acid is the livJ gene. In one embodiment, the at least one gene encoding a transporter of branched chain amino acid is the livH gene. In one embodiment, the at least one gene encoding a transporter of branched chain amino acid is the livM gene. In one embodiment, the at least one gene encoding a transporter of branched chain amino acid is the livG gene. In one embodiment, the at least one gene encoding a transporter of branched chain amino acid is the livF gene. In one embodiment, the at least one gene encoding a transporter of a branched chain amino acid is the livKHMGF operon. In one embodiment, the at least one gene encoding a transporter of a branched chain amino acid is the livK gene. In another embodiment, the livKHMGF operon is an Escherichia coli livKHMGF operon. In another embodiment, the at least one gene encoding a transporter of a branched chain amino acid comprises the livKHMGF operon and the livJ gene. In one embodiment, the bacterial cell has been genetically engineered to comprise at least one heterologous gene encoding a LIV-I system. In one embodiment, the bacterial cell has been genetically engineered to comprise at least one heterologous gene encoding a LS system. In one embodiment, the bacterial cell has been genetically engineered to comprise at least one heterologous gene encoding a LIV-I system. In one embodiment, the bacterial cell has been genetically engineered to comprise at least one heterologous livJ gene, and at least one heterologous gene selected from the group consisting of livH, livM, livG, and livF. In one embodiment, the bacterial cell has been genetically engineered to comprise at least one heterologous livK gene, and at least one heterologous gene selected from the group consisting of livH, livM, livG, and livF. In any of these embodiments, the bacterial cell may comprise more than one copy of any of one or more of these gene sequences. In any of these embodiments, the bacterial cell may over-express any one or more of these gene sequences. In any of these embodiments, the bacterial cell may further comprise gene sequence(s) encoding one or more additional BCAA transporters, e.g., brnQ transporter.

The present disclosure further provides genes encoding functional fragments of a transporter of a branched chain amino acid or functional variants of an importer of a branched chain amino acid. As used herein, the term “functional fragment thereof” or “functional variant thereof” of a transporter of a branched chain amino acid relates to an element having qualitative biological activity in common with the wild-type transporter of a branched chain amino acid from which the fragment or variant was derived. For example, a functional fragment or a functional variant of a mutated transporter of a branched chain amino acid protein is one which retains essentially the same ability to import leucine into the bacterial cell as does the importer protein from which the functional fragment or functional variant was derived. In one embodiment, the recombinant bacterial cell disclosed herein comprises at least one heterologous gene encoding a functional fragment of a transporter of branched chain amino acid. In another embodiment, the recombinant bacterial cell disclosed herein comprises a heterologous gene encoding a functional variant of a transporter of branched chain amino acid.

Assays for testing the activity of an importer of a branched chain amino acid, a functional variant of a transporter of a branched chain amino acid, or a functional fragment of a transporter of a branched chain amino acid are well known to one of ordinary skill in the art. For example, import of a branched chain amino acid may be determined using the methods as described in Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of each of which are expressly incorporated by reference herein.

In one embodiment, the genes encoding the transporter of a branched chain amino acid have been codon-optimized for use in the host organism. In one embodiment, the genes encoding the importer of a branched chain amino acid have been codon-optimized for use in Escherichia coli.

The present disclosure also encompasses genes encoding a transporter of a branched chain amino acid, e.g., a transporter of leucine, comprising amino acids in its sequence that are substantially the same as an amino acid sequence described herein Amino acid sequences that are substantially the same as the sequences described herein include sequences comprising conservative amino acid substitutions, as well as amino acid deletions and/or insertions.

In some embodiments, the at least one gene encoding a transporter of a branched chain amino acid, e.g., livKHMGF, is mutagenized; mutants exhibiting increased branched chain amino acid, e.g., leucine, transport are selected; and the mutagenized at least one gene encoding a transporter of a branched chain amino acid, e.g., livKHMGF, is isolated and inserted into the bacterial cell. In some embodiments, the at least one gene encoding a transporter of a branched chain amino acid, e.g., livKHMGF, is mutagenized; mutants exhibiting decreased branched chain amino acid, e.g., leucine, transport are selected; and the mutagenized at least one gene encoding a transporter of a branched chain amino acid, e.g., livKHMGF, is isolated and inserted into the bacterial cell. The importer modifications described herein may be present on a plasmid or chromosome.

In one embodiment, the livKHMGF operon has at least about 80% identity with the uppercase sequence of SEQ ID NO:5. Accordingly, in one embodiment, the livKHMGF operon has at least about 90% identity with the uppercase sequence of SEQ ID NO:5. Accordingly, in one embodiment, the livKHMGF operon has at least about 95% identity with the uppercase sequence of SEQ ID NO:5. Accordingly, in one embodiment, the livKHMGF operon has at least about 85%, 86%, 87%, 88%, 89%, 90%, 91%, 92%, 93%, 94%, 95%, 96%, 97%, 98%, or 99% identity with the uppercase sequence of SEQ ID NO:5. In another embodiment, the livKHMGF operon comprises the uppercase sequence of SEQ ID NO:5. In yet another embodiment the livKHMGF operon consists of the uppercase sequence of SEQ ID NO:5.

In some embodiments, the bacterial cell comprises a heterologous gene encoding a branched chain amino acid catabolism enzyme operably linked to a first promoter and at least one heterologous gene encoding a transporter of a branched chain amino acid. In some embodiments, the at least one heterologous gene encoding a transporter of a branched chain amino acid is operably linked to the first promoter. In other embodiments, the at least one heterologous gene encoding a transporter of a branched chain amino acid is operably linked to a second promoter. In one embodiment, the at least one gene encoding a transporter of a branched chain amino acid is directly operably linked to the second promoter. In another embodiment, the at least one gene encoding a transporter of a branched chain amino acid is indirectly operably linked to the second promoter.

In some embodiments, expression of at least one gene encoding a transporter of a branched chain amino acid, e.g., livKHMGF and/or brnQ, is controlled by a different promoter than the promoter that controls expression of the gene encoding the branched chain amino acid catabolism enzyme. In some embodiments, expression of the at least one gene encoding a transporter of a branched chain amino acid, e.g., livKHMGF and/or brnQ, is controlled by the same promoter that controls expression of the branched chain amino acid catabolism enzyme. In some embodiments, at least one gene encoding a transporter of a branched chain amino acid, e.g., livKHMGF and/or brnQ, and the branched chain amino acid catabolism enzyme are divergently transcribed from a promoter region. In some embodiments, expression of each of genes encoding the at least one gene encoding a transporter of a branched chain amino acid, e.g., livKHMGF and/or brnQ, and the gene encoding the branched chain amino acid catabolism enzyme is controlled by different promoters.

In one embodiment, the at least one gene encoding a transporter of a branched chain amino acid is not operably linked to its native promoter. In some embodiments, the at least one gene encoding the transporter of a branched chain amino acid, e.g., livKHMGF, is controlled by its native promoter. In some embodiments, the at least one gene encoding a transporter of a branched chain amino acid, e.g., livKHMGF and/or brnQ, is controlled by an inducible promoter. In some embodiments, the at least one gene encoding a transporter of a branched chain amino acid, e.g., livKHMG and/or brnQF, is controlled by a promoter that is stronger than its native promoter. In some embodiments, the at least one gene encoding a transporter of a branched chain amino acid, e.g., livKHMGF, is controlled by a constitutive promoter.

In another embodiment, the promoter is an inducible promoter. Inducible promoters are described in more detail infra.

In one embodiment, the at least one gene encoding a transporter of a branched chain amino acid is located on a plasmid in the bacterial cell. In another embodiment, the at least one gene encoding a transporter of a branched chain amino acid is in the chromosome of the bacterial cell. In yet another embodiment, a native copy of the at least one gene encoding a transporter of a branched chain amino acid is located in the chromosome of the bacterial cell, and a copy of at least one gene encoding a transporter of a branched chain amino acid from a different species of bacteria is located on a plasmid in the bacterial cell. In yet another embodiment, a native copy of the at least one gene encoding a transporter of a branched chain amino acid is located on a plasmid in the bacterial cell, and a copy of at least one gene encoding a transporter of a branched chain amino acid from a different species of bacteria is located on a plasmid in the bacterial cell. In yet another embodiment, a native copy of the at least one gene encoding a transporter of a branched chain amino acid is located in the chromosome of the bacterial cell, and a copy of the at least one gene encoding an importer of a branched chain amino acid from a different species of bacteria is located in the chromosome of the bacterial cell.

In some embodiments, the at least one native gene encoding a transporter, e.g., livKHMG and/or brnQF, in the bacterial cell is not modified, and one or more additional copies of the native transporter, e.g., livKHMGF and/or brnQ, are inserted into the genome. In some embodiments, the at least one native gene encoding a transporter, e.g., livKHMG and/or brnQF, in the bacterial cell is not modified, and one or more additional copies of the native transporter, e.g., livKHMGF and/or brnQ, are present on a plasmid, e.g., a high copy or low copy plasmid. In one embodiment, the one or more additional copies of the native a transporter, e.g., livKHMGF and/or brnQ, that is inserted into the genome are under the control of the same inducible promoter that controls expression of the gene encoding the branched chain amino acid catabolism enzyme, e.g., the FNR responsive promoter, or a different inducible promoter than the one that controls expression of the branched chain amino acid catabolism enzyme, or a constitutive promoter. In alternate embodiments, the at least one native gene encoding the transporter, e.g., livKHMGF and/or brnQ, is not modified, and one or more additional copies of the transporter, e.g., livKHMGF, from a different bacterial species is inserted into the genome of the bacterial cell. In one embodiment, the one or more additional copies of the transporter inserted into the genome of the bacterial cell are under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter.

In some embodiments, at least one native gene encoding the transporter, e.g., livKHMGF and/or brnQ, in the genetically modified bacteria is not modified, and one or more additional copies of at least one native gene encoding the transporter, e.g., livKHMGF and/or brnQ, are present in the bacterial cell on a plasmid. In one embodiment, the at least one native gene encoding the transporter e.g., livKHMGF and/or brnQ, present in the bacterial cell on a plasmid is under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter. In alternate embodiments, the at least one native gene encoding the transporter, e.g., livKHMGF and/or brnQ, is not modified, and a copy of at least one gene encoding the transporter, e.g., livKHMGF and/or brnQ, from a different bacterial species is present in the bacteria on a plasmid. In one embodiment, the copy of at least one gene encoding the transporter, e.g., livKHMGF and/or brnQ, from a different bacterial species is under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter.

In some embodiments, the bacterium is E. coli Nissle, and the at least one native gene encoding the transporter, e.g., livKHMGF and/or brnQ, in E. coli Nissle is not modified; one or more additional copies at least one native gene encoding the transporter, e.g., livKHMGF and/or brnQ, from E. coli Nissle is inserted into the E. coli Nissle genome under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter. In an alternate embodiment, the at least one native gene encoding the a transporter, e.g., livKHMGF and/or brnQ in E. coli Nissle is not modified, and a copy of at least one gene encoding the transporter, e.g., livKHMGF and/or brnQ, from a different bacterial species is inserted into the E. coli Nissle genome under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter.

In some embodiments, the bacterial cell is E. coli Nissle, and the at least one native gene encoding the transporter, e.g., livKHMGF and/or brnQ, in E. coli Nissle is not modified; one or more additional copies the at least one native gene encoding the transporter, e.g., livKHMGF and/or brnQ, E. coli Nissle is present in the bacterium on a plasmid and under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter. In an alternate embodiment, the at least one native gene encoding the transporter, e.g., livKHMGF, in E. coli Nissle is not modified, and a copy of at least one native gene encoding the transporter, e.g., livKHMGF and/or brnQ, from a different bacterial species of are present in the bacterium on a plasmid and under the control of the same inducible promoter that controls expression of the gene encoding the branched chain amino acid catabolism enzyme, e.g., the FNR responsive promoter, or a different inducible promoter than the one that controls expression of the gene encoding the branched chain amino acid catabolism enzyme, or a constitutive promoter.

In one embodiment, when the transporter of a branched chain amino acid is expressed in the recombinant bacterial cells disclosed herein, the bacterial cells import 10% more branched chain amino acids, e.g., leucine, into the bacterial cell when the transporter is expressed as compared to unmodified bacteria of the same bacterial subtype under the same conditions. In another embodiment, when the transporter of a branched chain amino acid is expressed in the recombinant bacterial cells disclosed herein, the bacterial cells import 20%, 30%, 40%, 50%, 60%, 70%, 80%, 90% or 100% more branched chain amino acids, e.g., leucine, into the bacterial cell when the transporter is expressed as compared with unmodified bacteria of the same bacterial subtype under the same conditions. In yet another embodiment, when the transporter of a branched chain amino acid is expressed in the recombinant bacterial cells disclosed herein, the bacterial cells import two-fold more branched chain amino acids, e.g., leucine, into the cell when the transporter is expressed than unmodified bacteria of the same bacterial subtype under the same conditions. In yet another embodiment, when the transporter of a branched chain amino acid is expressed in the recombinant bacterial cells disclosed herein, the bacterial cells import three-fold, four-fold, five-fold, six-fold, seven-fold, eight-fold, nine-fold, or ten-fold more branched chain amino acids, e.g., leucine, into the cell when a transporter is expressed as compared with unmodified bacteria of the same bacterial subtype under the same conditions.

Exporter of a Branched Chain Amino Acid

The bacterial cells disclosed herein may comprise a genetic modification that inhibits or decreases the export of a branched chain amino acid and/or its corresponding alpha keto acid or other metabolite from the bacterial cell. Knocking-out or reducing export of one or more branched chain amino acids from a bacterial cell allows the bacterial cell to more efficiently retain and catabolize exogenous branched chain amino acids and/or their alpha-keto acid counterparts or other metabolite counterparts in order to treat the diseases and disorders described herein. Any of the bacterial cells disclosed herein comprising gene sequence encoding one or more BCAA catabolism enzymes and/or one or more BCAA transporters may further a genetic modification that inhibits or decreases the export of a branched chain amino acid and/or its corresponding alpha keto acid or other metabolite from the bacterial cell.

The export of branched chain amino acids from bacterial cells is mediated by proteins well known to those of skill in the art. For example, one branched chain amino acid exporter, the leucine exporter LeuE has been characterized in Escherichia coli (Kutukova et al., FEBS Letters 579:4629-34 (2005); incorporated herein by reference). LeuE is encoded by the leuE gene in Escherichia coli (also known as yeaS). Additionally, a two-gene encoded exporter of the branched chain amino acids isoleucine, valine and leucine, denominated BrnFE was identified in the bacteria Corynebacterium glutamicum (Kennerknecht et al., J. Bacteriol. 184:3947-56 (2002); incorporated herein by reference). The BrnFE system is encoded by the Corynebacterium glutamicum genes brnF and brnE, and homologues of said genes have been identified in several organisms, including Agrobacterium tumefaciens, Achaeoglobus fulgidus, Bacillus subtilis, Deinococcus radiodurans, Escherichia coli, Haemophilus influenzae, Helicobacter pylori, Lactococcus lactis, Streptococcus pneumoniae, and Vibrio cholerae (see Kennerknecht et al., 2002).

The bacterial cells disclosed herein comprise a genetic modification that reduces export of a branched chain amino acid from the bacterial cell. Multiple distinct exporters of branched chain amino acids, e.g., leucine, are known in the art. In one embodiment, the recombinant bacterial cell disclosed herein comprises a genetic modification that reduces export of a branched chain amino acid from the bacterial cell, wherein the endogenous gene encoding an exporter of a branched chain amino acid is a leuE gene. In one embodiment, the recombinant bacterial cell disclosed herein comprises a genetic modification that reduces export of a branched chain amino acid from the bacterial cell, wherein the endogenous gene encoding an exporter of a branched chain amino acid is a bmF gene. In one embodiment, the recombinant bacterial cell disclosed herein comprises a genetic modification that reduces export of a branched chain amino acid from the bacterial cell, wherein the endogenous gene encoding an exporter of a branched chain amino acid is a bmE gene. In one embodiment, the recombinant bacterial cell disclosed herein comprises a genetic modification that reduces export of a branched chain amino acid from the bacterial cell and a heterologous gene encoding a branched chain amino acid catabolism enzyme. When the recombinant bacterial cells disclosed herein comprise a genetic modification that reduces export of a branched chain amino acid, the bacterial cells retain more branched chain amino acids, e.g., leucine, in the bacterial cell than unmodified bacteria of the same bacterial subtype under the same conditions. Thus, the recombinant bacteria comprising a genetic modification that reduces export of a branched chain amino acid may be used to retain more branched chain amino acids in the bacterial cell so that any branched chain amino acid catabolism enzyme expressed in the organism, e.g., co-expressed α-ketoisovalerate decarboxylase or co-expressed branched chain keto dehydrogenase, can catabolize the branched chain amino acids, e.g., leucine, to treat diseases associated with the catabolism of branched chain amino acids, including Maple Syrup Urine Disease (MSUD). In one embodiment, the recombinant bacteria further comprise a heterologous gene encoding an importer of a branched chain amino acid, e.g., a livKHMGF and/or brnQ gene.

In one embodiment, the genetic modification reduces export of a branched chain amino acid, e.g., leucine, from the bacterial cell. In one embodiment, the bacterial cell is from a bacterial genus or species that includes but is not limited to, Bacillus, Bacteroides, Bifidobacterium, Brevibacteria, Clostridium, Enterococcus, Escherichia, Lactobacillus, Lactococcus, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis. In another embodiment, the bacterial cell is an Escherichia coli bacterial cell. In another embodiment, the bacterial cell is an Escherichia coli strain Nissle bacterial cell.

In one embodiment, the genetic modification is a mutation in an endogenous gene encoding an exporter of a branched chain amino acid. In one embodiment, the genetic mutation is a deletion of the endogenous gene encoding an exporter, e.g., leuE, of a branched chain amino acid. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter, e.g., LeuE, having no activity, i.e., results in an exporter, e.g., LeuE, which cannot export a branched chain amino acid, e.g., lysine, from the bacterial cell.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of a branched chain amino acid. Mutagenesis and directed evolution methods are well known in the art for creating variants. See, e.g., U.S. Pat. No. 7,783,428; U.S. Pat. No. 6,586,182; U.S. Pat. No. 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated” as applied to a gene refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

Assays for testing the activity of an exporter of a branched chain amino acid, e.g., leucine, are well known to one of ordinary skill in the art. For example, export of a branched chain amino acid, such as leucine, may be determined using the methods described by Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of which are expressly incorporated herein by reference.

In another embodiment, the genetic modification is a mutation in a promoter of an endogenous gene encoding an exporter of a branched chain amino acid. In one embodiment, the genetic mutation results in decreased expression of the leuE gene. In one embodiment, leuE gene expression is reduced by about 50%, 75%, or 100%. In another embodiment, leuE gene expression is reduced about two-fold, three-fold, four-fold, or five-fold. In another embodiment, the genetic mutation completely inhibits expression of the leuE gene.

Assays for testing the level of expression of a gene, such as an exporter of a branched chain amino acid, e.g., leuE, are well known to one of ordinary skill in the art. For example, reverse-transcriptase polymerase chain reaction may be used to detect the level of mRNA expression of a gene. Alternatively, Western blots using antibodies directed against a protein may be used to determine the level of expression of the protein.

In another embodiment, the genetic modification is an overexpression of a repressor of an exporter of a branched chain amino acid. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

Reduction of Endogenous Bacterial Branched Chain Amino Acid Production

The bacterial cells disclosed herein may comprise a genetic modification that inhibits or decreases the biosynthesis of a branched chain amino acid and/or its corresponding alpha keto acid or other metabolite in the bacterial cell. Knocking-out or reducing production of endogenous branched chain amino acids in a bacterial cell allows the bacterial cell to more efficiently take up and catabolize exogenous branched chain amino acids and/or their alpha-keto acid counterparts or other metabolite counterparts in order to treat the diseases and disorders described herein. Knock-out or knock down of a gene encoding an enzyme required for branched chain amino acid biosynthesis creates an auxotroph, which requires the cell to import the branched chain amino acid or a metabolite to survive. Any of the bacterial cells disclosed herein comprising gene sequence encoding one or more BCAA catabolism enzymes and/or one or more BCAA transporters may further a genetic modification that inhibits or decreases the biosynthesis of a branched chain amino acid and/or its corresponding alpha keto acid or other metabolite in the bacterial cell.

As used herein, the term “branched chain amino acid biosynthesis” enzyme refers to an enzyme involved in the biosynthesis of a branched chain amino acid and/or its corresponding alpha-keto acid or other metabolite. Multiple distinct genes involved in biosynthetic pathways of branched chain amino acids, e.g., isoleucine, leucine, and valine, are known in the art. For example, the ilvC gene encodes a keto-acid reductoisomerase enzyme that catalyzes the conversion of acetohydroxy acids into dihydroxy valerates, which leads to the synthesis of the essential branched side chain amino acids valine and isoleucine (EC 1.1.1.86) and has been characterized in Escherichia coli (Wek and Hatfield, J. Biol. Chem. 261:2441-50 (1986), the entire contents of which are expressly incorporated herein by reference). Additionally, homologues of ilvC have been identified in several organisms, including Candida albicans, Oryza sativa, Saccharomyces cerevisiae, Pseudomonas aeruginosa, Corynebacterium glutamicum, and Spinacia oleracea.

In one embodiment, the genetic modification is a mutation in an endogenous gene encoding a protein that is involved in the biosynthesis of a branched chain amino acid or an alpha keto acid, e.g., ilvC. ilvC is an acetohydroxy acid isomeroreductase that is required for branched chain amino acid synthesis. In one embodiment, the genetic mutation is a deletion of an endogenous gene encoding a protein that is involved in the biosynthesis of a branched chain amino acid or an alpha-keto acid, or other BCAA metabolite, e.g., ilvC. In another embodiment, the genetic mutation results in an enzyme having reduced activity as compared to a wild-type enzyme. In one embodiment, the activity of the enzyme is reduced at least 50%, at least 75%, or at least 100%. In another embodiment, the activity of the enzyme is reduced at least two-fold, three-fold, four-fold, or five-fold. In another embodiment, the genetic mutation results in an enzyme, e.g., IlvC, having no activity, i.e., results in an enzyme, e.g., IlvC, which cannot catalyze the conversion of acetohydroxy acids into dihydroxy valerates, thereby inhibiting the endogenous synthesis of the branched chain amino acids valine and isoleucine in the recombinant bacterial cell.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of a branched chain amino acid. Mutagenesis and directed evolution methods are well known in the art for creating variants. See, e.g., U.S. Pat. No. 7,783,428; U.S. Pat. No. 6,586,182; U.S. Pat. No. 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated,” as applied to a gene, refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

Assays for testing the activity of enzymes involved in the biosynthesis of branched chain amino acids and/or alpha-keto acids, and/or other BCAA metabolite e.g., ilvC, are well known to one of ordinary skill in the art. For example, the activity of a ketol-acid reductoisomerase enzyme may be determined using the methods described by Durner et al., Plant Physiol., 103:903-910, 1993, the entire contents of which are expressly incorporated herein by reference.

In another embodiment, the genetic modification is a mutation in a promoter of an endogenous gene encoding the branched chain amino acid biosynthesis enzyme. In one embodiment, the genetic mutation results in decreased expression of the branched chain amino acid biosynthesis enzyme gene. In one embodiment, gene expression is reduced by about 50%, 75%, or 100%. In another embodiment, gene expression is reduced about two-fold, three-fold, four-fold, or five-fold. In another embodiment, the genetic mutation completely inhibits expression of the gene.

Assays for testing the level of expression of a gene, such as ilvC, are well known to one of ordinary skill in the art. For example, reverse-transcriptase polymerase chain reaction may be used to detect the level of mRNA expression of a gene. Alternatively, Western blots using antibodies directed against a protein may be used to determine the level of expression of the protein.

In another embodiment, the genetic modification is an overexpression of a repressor of a branched chain amino acid biosynthesis gene. In one embodiment, the overexpression of the repressor of the gene is caused by a mutation which renders the promoter of the repressor constitutively active. In another embodiment, the overexpression of the repressor of the branched chain amino acid biosynthesis gene is caused by the insertion of an inducible promoter in front of the repressor so that the expression of the repressor can be induced. Inducible promoters are described in more detail herein.

Inducible Promoters

In some embodiments, the bacterial cell comprises a stably maintained plasmid or chromosome carrying the gene encoding the branched chain amino acid catabolism enzyme, e.g., kivD, leuDH, ilvE, L-AAD, BCKD, adh2, PadA, and/or YqhD such that the branched chain amino acid catabolism enzyme can be expressed in the host cell, and the host cell is capable of survival and/or growth in vitro, e.g., in medium, and/or in vivo, e.g., in the gut. In some embodiments, bacterial cell comprises two or more distinct branched chain amino acid catabolism enzymes, e.g., kivD, leuDH, ilvE, BCKD, L-AAD, PadA, adh2, PadA and/or YqhD genes. In some embodiments, the genetically engineered bacteria comprise multiple copies of the same branched chain amino acid catabolism enzyme gene. In some embodiments, the genetically engineered bacteria comprise multiple copies of different branched chain amino acid catabolism enzyme genes. In some embodiments, the gene encoding the branched chain amino acid catabolism enzyme is present on a plasmid and operably linked to a directly or indirectly inducible promoter. In some embodiments, the gene encoding the branched chain amino acid catabolism enzyme is present on a plasmid and operably linked to a promoter that is induced under low-oxygen or anaerobic conditions. In some embodiments, the gene encoding the branched chain amino acid catabolism enzyme is present on a chromosome and operably linked to a directly or indirectly inducible promoter. In some embodiments, the gene encoding the branched chain amino acid catabolism enzyme is present in the chromosome and operably linked to a promoter that is induced under low-oxygen or anaerobic conditions. In some embodiments, the gene encoding the branched chain amino acid catabolism enzyme is present on a plasmid and operably linked to a promoter that is induced by exposure to tetracycline or arabinose.

In some embodiments, the bacterial cell comprises a stably maintained plasmid or chromosome carrying the at least one gene encoding a transporter of a branched chain amino acid, e.g., livKHMGF and/or brnQ, such that the transporter, e.g., LivKHMGF and/or brnQ, can be expressed in the host cell, and the host cell is capable of survival and/or growth in vitro, e.g., in medium, and/or in vivo, e.g., in the gut. In some embodiments, bacterial cell comprises two or more distinct copies of the at least one gene encoding a transporter of a branched chain amino acid, e.g., livKHMGF and/or brnQ. In some embodiments, the genetically engineered bacteria comprise multiple copies of the same at least one gene encoding a transporter of a branched chain amino acid, e.g., livKHMGF and/or brnQ. In some embodiments, the at least one gene encoding a transporter of a branched chain amino acid, e.g., livKHMGF and/or brnQ, is present on a plasmid and operably linked to a directly or indirectly inducible promoter. In some embodiments, the at least one gene encoding a transporter of a branched chain amino acid, e.g., livKHMGF and/or brnQ, is present on a plasmid and operably linked to a promoter that is induced under low-oxygen or anaerobic conditions. In some embodiments, the at least one gene encoding a transporter of a branched chain amino acid, e.g., livKHMGF and/or brnQ, is present on a chromosome and operably linked to a directly or indirectly inducible promoter. In some embodiments, the at least one gene encoding a transporter of a branched chain amino acid, e.g., livKHMGF and/or brnQ, is present in the chromosome and operably linked to a promoter that is induced under low-oxygen or anaerobic conditions. In some embodiments, the at least one gene encoding a transporter of a branched chain amino acid, e.g., livKHMGF and/or brnQ, is present on a plasmid and operably linked to a promoter that is induced by exposure to tetracycline.

In some embodiments, the promoter that is operably linked to the gene encoding the branched chain amino acid catabolism enzyme and the promoter that is operably linked to the gene encoding the transporter of a branched chain amino acid, e.g., livKHMGF and/or brnQ, is directly induced by exogenous environmental conditions. In some embodiments, the promoter that is operably linked to the gene encoding the branched chain amino acid catabolism enzyme and the promoter that is operably linked to the gene encoding the transporter of a branched chain amino acid, e.g., livKHMGF and/or brnQ, is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal, e.g., propionate. In some embodiments, the promoter is directly or indirectly induced by a molecule that is co-administered with the bacterial cell.

In some embodiments, the bacterial cell comprises a stably maintained plasmid or chromosome carrying the at least one gene encoding a branched chain amino acid binding protein, e.g., livJ, such that the BCAA binding protein, e.g., livJ, can be expressed in the host cell, and the host cell is capable of survival and/or growth in vitro, e.g., in medium, and/or in vivo, e.g., in the gut. In some embodiments, bacterial cell comprises two or more distinct copies of the at least one gene encoding a BCAA binding protein of a branched chain amino acid, e.g., livJ. In some embodiments, the genetically engineered bacteria comprise multiple copies of the same at least one gene encoding a binding protein of a branched chain amino acid, e.g., livJ. In some embodiments, the at least one gene encoding a binding protein of a branched chain amino acid, e.g., livJ, is present on a plasmid and operably linked to a directly or indirectly inducible promoter. In some embodiments, the at least one gene encoding a binding protein of a branched chain amino acid, e.g., livJ, is present on a plasmid and operably linked to a promoter that is induced under low-oxygen or anaerobic conditions. In some embodiments, the at least one gene encoding a binding protein of a branched chain amino acid, e.g., livJ, is present on a chromosome and operably linked to a directly or indirectly inducible promoter. In some embodiments, the at least one gene encoding a binding protein of a branched chain amino acid, e.g., livJ, is present in the chromosome and operably linked to a promoter that is induced under low-oxygen or anaerobic conditions. In some embodiments, the at least one gene encoding a binding protein of a branched chain amino acid, e.g., livJ, is present on a plasmid and operably linked to a promoter that is induced by exposure to tetracycline.

In some embodiments, the promoter that is operably linked to the gene encoding the branched chain amino acid catabolism enzyme and the promoter that is operably linked to the gene encoding the binding protein of a branched chain amino acid, e.g., livJ, is directly induced by exogenous environmental conditions. In some embodiments, the promoter that is operably linked to the gene encoding the branched chain amino acid catabolism enzyme and the promoter that is operably linked to the gene encoding the binding protein of a branched chain amino acid, e.g., livJ, is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal, e.g., propionate. In some embodiments, the promoter is directly or indirectly induced by a molecule that is co-administered with the bacterial cell.

In certain embodiments, the bacterial cell comprises a gene encoding a branched chain amino acid catabolism enzyme, e.g., kivD, leuDH, ilvE, BCKD, L-AAD, padA, yqhD and/or adh2, is expressed under the control of the fumarate and nitrate reductase regulator (FNR) promoter. In certain embodiments, the bacterial cell comprises at least one gene encoding a transporter of a branched chain amino acid, e.g., livKHMGF and/or brnQ, is expressed under the control of the fumarate and nitrate reductase regulator (FNR) promoter. In certain embodiments, the bacterial cell comprises at least one gene encoding a binding protein of a branched chain amino acid, e.g., livJ, is expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive.

FNR responsive promoters include, but are not limited to, the FNR responsive promoters listed in the chart, below. Underlined sequences are predicted ribosome binding sites, and bolded sequences are restriction sites used for cloning.

TABLE 4
FNR responsive promoters

| FNR Responsive | |
|---|---|
| Promoter | Sequence |
| SEQ ID NO: 14 | GTCAGCATAACACCCTGACCTCTCATTAATTGTTCATGCCGGGCGGCACTATCGTCGTCCGGCCT |
| | TTTCCTCTCTTACTCTGCTACGTACATCTATTTCTATAAATCCGTTCAATTTGTCTGTTTTTTGCACA |
| | AACATGAAATATCAGACAATTCCGTGACTTAAGAAAATTTATACAAATCAGCAATATACCCCTTA |
| | AGGAGTATATAAAGGTGAATTTGATTTACATCAATAAGCGGGGTTGCTGAATCGTTAAGGTAGG |
| | CGGTAATAGAAAAGAAATCGAGGCAAAA |
| SEQ ID NO: 15 | ATTTCCTCTCATCCCATCCGGGGTGAGAGTCTTTTCCCCCGACTTATGGCTCATGCATGCATCAAA |
| | AAAGATGTGAGCTTGATCAAAAACAAAAAATATTTCACTCGACAGGAGTATTTATATTGCGCCCG |
| | TTACGTGGGCTTCGACTGTAAATCAGAAAGGAGAAAACACCT |
| SEQ ID NO: 16 | GTCAGCATAACACCCTGACCTCTCATTAATTGTTCATGCCGGGCGGCACTATCGTCGTCCGGCCT |
| | TTTCCTCTCTTACTCTGCTACGTACATCTATTTCTATAAATCCGTTCAATTTGTCTGTTTTTTGCACA |
| | AACATGAAATATCAGACAATTCCGTGACTTAAGAAAATTTATACAAATCAGCAATATACCCCTTA |
| | AGGAGTATATAAAGGTGAATTTGATTTACATCAATAAGCGGGGTTGCTGAATCGTTAAGGATCC |
| | CTCTAGAAATAATTTTGTTTAACTTTAAGAAGGAGATATACAT |
| SEQ ID NO: 17 | CATTTCCTCTCATCCCATCCGGGGTGAGAGTCTTTTCCCCCGACTTATGGCTCATGCATGCATCAA |
| | AAAAGATGTGAGCTTGATCAAAAACAAAAAATATTTCACTCGACAGGAGTATTTATATTGCGCCC |
| | GGATCCCTCTAGAAATAATTTTGTTTAACTTTAAGAAGGAGATATACAT |
| SEQ ID NO: 18 | AGTTGTTCTTATTGGTGGTGTTGCTTTATGGTTGCATCGTAGTAAATGGTTGTAACAAAAGCAAT |
| | TTTTCCGGCTGTCTGTATACAAAAACGCCGTAAAGTTTGAGCGAAGTCAATAAACTCTCTACCCA |
| | TTCAGGGCAATATCTCTCTTGGATCCCTCTAGAAATAATTTTGTTTAACTTTAAGAAGGAGATATA |
| | CAT |

In one embodiment, the FNR responsive promoter comprises SEQ ID NO:14. In another embodiment, the FNR responsive promoter comprises SEQ ID NO:15. In another embodiment, the FNR responsive promoter comprises SEQ ID NO:16. In another embodiment, the FNR responsive promoter comprises SEQ ID NO:17. In yet another embodiment, the FNR responsive promoter comprises SEQ ID NO:18. Additional FNR responsive promoters are shown below.

TABLE 5
FNR Promoter Sequences

| SEQ ID NO | FNR-responsive regulatory region Sequence |
|---|---|
| SEQ ID NO: 80 | ATCCCCATCACTCTTGATGGAGATCAATTCCCCAAGCTGCTAGAGCGTTA |
| | CCTTGCCCTTAAACATTAGCAATGTCGATTTATCAGAGGGCCGACAGGCT |
| | CCCACAGGAGAAAACCG |
| SEQ ID NO: 81 | CTCTTGATCGTTATCAATTCCCACGCTGTTTCAGAGCGTTACCTTGCCCT |
| | TAAACATTAGCAATGTCGATTTATCAGAGGGCCGACAGGCTCCCACAGGA |
| | GAAAACCG |
| nirB1 | GTCAGCATAACACCCTGACCTCTCATTAATTGTTCATGCCGGGCGGCACT |
| SEQ ID NO: 82 | ATCGTCGTCCGGCCTTTTCCTCTCTTACTCTGCTACGTACATCTATTTCT |
| | ATAAATCCGTTCAATTTGTCTGTTTTTTGCACAAACATGAAATATCAGAC |
| | AATTCCGTGACTTAAGAAAATTTATACAAATCAGCAATATACCCCTTAAG |
| | GAGTATATAAAGGTGAATTTGATTTACATCAATAAGCGGGGTTGCTGAAT |
| | CGTTAAGGTAGGCGGTAATAGAAAAGAAATCGAGGCAAAA |
| nirB2 | CGGCCCGATCGTTGAACATAGCGGTCCGCAGGCGGCACTGCTTACAGCAA |
| SEQ ID NO: 83 | ACGGTCTGTACGCTGTCGTCTTTGTGATGTGCTTCCTGTTAGGTTTCGTC |
| | AGCCGTCACCGTCAGCATAACACCCTGACCTCTCATTAATTGCTCATGCC |
| | GGACGGCACTATCGTCGTCCGGCCTTTTCCTCTCTTCCCCCGCTACGTGC |
| | ATCTATTTCTATAAACCCGCTCATTTTGTCTATTTTTTGCACAAACATGA |
| | AATATCAGACAATTCCGTGACTTAAGAAAATTTATACAAATCAGCAATAT |
| | ACCCATTAAGGAGTATATAAAGGTGAATTTGATTTACATCAATAAGCGGG |
| | GTTGCTGAATCGTTAAGGTAGGCGGTAATAGAAAAGAAATCGAGGCAAAA |
| | atgtttgtttaactttaagaaggagatatacat |
| nirB3 | GTCAGCATAACACCCTGACCTCTCATTAATTGCTCATGCCGGACGGCACT |
| SEQ ID NO: 84 | ATCGTCGTCCGGCCTTTTCCTCTCTTCCCCCGCTACGTGCATCTATTTCT |
| | ATAAACCCGCTCATTTTGTCTATTTTTTGCACAAACATGAAATATCAGAC |
| | AATTCCGTGACTTAAGAAAATTTATACAAATCAGCAATATACCCATTAAG |
| | GAGTATATAAAGGTGAATTTGATTTACATCAATAAGCGGGGTTGCTGAAT |
| | CGTTAAGGTAGGCGGTAATAGAAAAGAAATCGAGGCAAAA |
| ydfZ | ATTTCCTCTCATCCCATCCGGGGTGAGAGTCTTTTCCCCCGACTTATGGC |
| SEQ ID NO: 85 | TCATGCATGCATCAAAAAAGATGTGAGCTTGATCAAAAACAAAAAATATT |
| | TCACTCGACAGGAGTATTTATATTGCGCCCGTTACGTGGGCTTCGACTGT |
| | AAATCAGAAAGGAGAAAACACCT |
| nirB + RBS | GTCAGCATAACACCCTGACCTCTCATTAATTGTTCATGCCGGGCGGCACT |
| SEQ ID NO: 86 | ATCGTCGTCCGGCCTTTTCCTCTCTTACTCTGCTACGTACATCTATTTCT |
| | ATAAATCCGTTCAATTTGTCTGTTTTTTGCACAAACATGAAATATCAGAC |
| | AATTCCGTGACTTAAGAAAATTTATACAAATCAGCAATATACCCCTTAAG |
| | GAGTATATAAAGGTGAATTTGATTTACATCAATAAGCGGGGTTGCTGAAT |
| | CGTTAAGGATCCCTCTAGAAATAATTTTGTTTAACTTTAAGAAGGAGATA |
| | TACAT |
| ydfZ + RBS | CATTTCCTCTCATCCCATCCGGGGTGAGAGTCTTTTCCCCCGACTTATGG |
| SEQ ID NO: 87 | CTCATGCATGCATCAAAAAAGATGTGAGCTTGATCAAAAACAAAAAATAT |
| | TTCACTCGACAGGAGTATTTATATTGCGCCCGGATCCCTCTAGAAATAAT |
| | TTTGTTTAACTTTAAGAAGGAGATATACAT |
| fnrS1 | AGTTGTTCTTATTGGTGGTGTTGCTTTATGGTTGCATCGTAGTAAATGGT |
| SEQ ID NO: 88 | TGTAACAAAAGCAATTTTTCCGGCTGTCTGTATACAAAAACGCCGTAAAG |
| | TTTGAGCGAAGTCAATAAACTCTCTACCCATTCAGGGCAATATCTCTCTT |
| | GGATCCCTCTAGAAATAATTTTGTTTAACTTTAAGAAGGAGATATACAT |
| fnrS2 | AGTTGTTCTTATTGGTGGTGTTGCTTTATGGTTGCATCGTAGTAAATGGT |
| SEQ ID NO: 89 | TGTAACAAAAGCAATTTTTCCGGCTGTCTGTATACAAAAACGCCGCAAAG |
| | TTTGAGCGAAGTCAATAAACTCTCTACCCATTCAGGGCAATATCTCTCTT |
| | GGATCCAAAGTGAACTCTAGAAATAATTTTGTTTAACTTTAAGAAGGAGA |
| | TATACAT |
| nirB + crp | TCGTCTTTGTGATGTGCTTCCTGTTAGGTTTCGTCAGCCGTCACCGTCAG |
| SEQ ID NO: 90 | CATAACACCCTGACCTCTCATTAATTGCTCATGCCGGACGGCACTATCGT |
| | CGTCCGGCCTTTTCCTCTCTTCCCCCGCTACGTGCATCTATTTCTATAAA |
| | CCCGCTCATTTTGTCTATTTTTTGCACAAACATGAAATATCAGACAATTC |
| | CGTGACTTAAGAAAATTTATACAAATCAGCAATATACCCATTAAGGAGTA |
| | TATAAAGGTGAATTTGATTTACATCAATAAGCGGGGTTGCTGAATCGTTA |
| | AGGTAGaaatgtgatctagttcacatttGCGGTAATAGAAAAGAAATCGA |
| | GGCAAAAatgtttgtttaactttaagaaggagatatacat |
| fnrS + crp | AGTTGTTCTTATTGGTGGTGTTGCTTTATGGTTGCATCGTAGTAAATGGT |
| SEQ ID NO: 143 | TGTAACAAAAGCAATTTTTCCGGCTGTCTGTATACAAAAACGCCGCAAAG |
| | TTTGAGCGAAGTCAATAAACTCTCTACCCATTCAGGGCAATATCTCTCaa |
| | atgtgatctagttcacattttttgtttaactttaagaaggagatatacat |

In some embodiments, multiple distinct FNR nucleic acid sequences are inserted in the genetically engineered bacteria. In alternate embodiments, the genetically engineered bacteria comprise a gene encoding a branched chain amino acid catabolism enzyme, e.g., kivD or BCKD or other enzyme disclosed herein, is expressed under the control of an alternate oxygen level-dependent promoter, e.g., DNR (Trunk et al., 2010) or ANR (Ray et al., 1997). In alternate embodiments, the genetically engineered bacteria comprise at least one gene encoding a transporter of a branched chain amino acid, e.g., livKHMGF and/or brnQ, is expressed under the control of an alternate oxygen level-dependent promoter, e.g., DNR (Trunk et al., 2010) or ANR (Ray et al., 1997). In alternate embodiments, the genetically engineered bacteria comprise at least one gene encoding a binding protein of a branched chain amino acid, e.g., livJ, is expressed under the control of an alternate oxygen level-dependent promoter, e.g., DNR (Trunk et al., 2010) or ANR (Ray et al., 1997). In these embodiments, catabolism of the branched chain amino acid, e.g., leucine, is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene encoding the branched chain amino acid catabolism enzyme, and/or the at least one gene encoding a transporter of a branched chain amino acid, e.g., livKHMGF and/or brnQ, and/or the at least one gene encoding a binding protein of a branched chain amino acid in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the branched chain amino acid catabolism enzyme, and/or the at least one gene encoding a transporter of a branched chain amino acid, e.g., livKHMGF and/or brnQ, and/or the at least one gene encoding a binding protein of a branched chain amino acid in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the branched chain amino acid catabolism enzyme, and/or the at least one gene encoding a transporter of a branched chain amino acid, e.g., livKHMGF and/or brnQ, and/or the at least one gene encoding a binding protein of a branched chain amino acid in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., 2006).

In some embodiments, the bacterial cells disclosed herein comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the branched chain amino acid catabolism enzyme are present on different plasmids. In some embodiments, the gene encoding the oxygen level-sensing transcriptional regulator and the gene encoding the branched chain amino acid catabolism enzyme and/or the at least one gene encoding a transporter of a branched chain amino acid and/or the at least one gene encoding a binding protein of a branched chain amino acid are present on different plasmids. In some embodiments, the gene encoding the oxygen level-sensing transcriptional regulator and the gene encoding the branched chain amino acid catabolism enzyme and/or the at least one gene encoding a transporter of a branched chain amino acid and/or the at least one gene encoding a binding protein of a branched chain amino acid are present on the same plasmid.

In some embodiments, the gene encoding the oxygen level-sensing transcriptional regulator is present on a chromosome. In some embodiments, the gene encoding the oxygen level-sensing transcriptional regulator and the gene encoding the gene encoding the branched chain amino acid catabolism enzyme and/or the at least one gene encoding a transporter of a branched chain amino acid and/or the at least one gene encoding a binding protein of a branched chain amino acid are present on different chromosomes. In some embodiments, the gene encoding the oxygen level-sensing transcriptional regulator and the gene encoding the branched chain amino acid catabolism enzyme and/or the at least one gene encoding a transporter of a branched chain amino acid and/or the at least one gene encoding a binding protein of a branched chain amino acid are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the branched chain amino acid catabolism enzyme and/or BCAA transporter and/or BCAA binding protein. In some embodiments, expression of the transcriptional regulator is controlled by the same promoter that controls expression of the branched chain amino acid catabolism enzyme and/or BCAA transporter and/or BCAA binding protein. In some embodiments, the transcriptional regulator and the branched chain amino acid catabolism enzyme are divergently transcribed from a promoter region.

RNS-Dependent Regulation

In some embodiments, the genetically engineered bacteria comprise a gene encoding a branched chain amino acid catabolism enzyme that is expressed under the control of an inducible promoter. In some embodiments, the genetically engineered bacterium that expresses a branched chain amino acid catabolism enzyme and/or BCAA transporter and/or BCAA binding protein is under the control of a promoter that is activated by inflammatory conditions. In one embodiment, the gene for producing the branched chain amino acid catabolism enzyme and/or BCAA transporter and/or BCAA binding protein is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO.), peroxynitrite or peroxynitrite anion (ONOO—), nitrogen dioxide (.NO2), dinitrogen trioxide (N2O3), peroxynitrous acid (ONOOH), and nitroperoxycarbonate (ONOOCO2-) (unpaired electrons denoted by .). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene or genes, e.g., a branched chain amino acid catabolism enzymegene sequence(s), e.g., any of the amino acid catabolism enzymes described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

As used herein, “RNS-derepressible regulatory region” refers to a nucleic acid sequence to which one or more RNS-sensing transcription factors is capable of binding, wherein the binding of the corresponding transcription factor represses downstream gene expression; in the presence of RNS, the transcription factor does not bind to and does not repress the regulatory region. In some embodiments, the RNS-derepressible regulatory region comprises a promoter sequence. The RNS-derepressible regulatory region may be operatively linked to a gene or genes, e.g., a branched chain amino acid catabolism enzymegene sequence(s), BCAA transporter sequence(s), BCAA binding protein(s). For example, in the presence of RNS, a transcription factor senses RNS and no longer binds to and/or represses the regulatory region, thereby derepressing an operatively linked gene sequence or gene cassette. Thus, RNS derepresses expression of the gene or genes.

As used herein, “RNS-repressible regulatory region” refers to a nucleic acid sequence to which one or more RNS-sensing transcription factors is capable of binding, wherein the binding of the corresponding transcription factor represses downstream gene expression; in the presence of RNS, the transcription factor binds to and represses the regulatory region. In some embodiments, the RNS-repressible regulatory region comprises a promoter sequence. In some embodiments, the transcription factor that senses RNS is capable of binding to a regulatory region that overlaps with part of the promoter sequence. In alternate embodiments, the transcription factor that senses RNS is capable of binding to a regulatory region that is upstream or downstream of the promoter sequence. The RNS-repressible regulatory region may be operatively linked to a gene sequence or gene cassette. For example, in the presence of RNS, a transcription factor senses RNS and binds to a corresponding RNS-repressible regulatory region, thereby blocking expression of an operatively linked gene sequence or gene sequences. Thus, RNS represses expression of the gene or gene sequences.

As used herein, a “RNS-responsive regulatory region” refers to a RNS-inducible regulatory region, a RNS-repressible regulatory region, and/or a RNS-derepressible regulatory region. In some embodiments, the RNS-responsive regulatory region comprises a promoter sequence. Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 6.

TABLE 6
Examples of RNS-sensing transcription
factors and RNS-responsive genes

| RNS-sensing | | |
|---|---|---|
| transcription | Primarily capable | Examples of responsive genes, |
| factor: | of sensing: | promoters, and/or regulatory regions: |
| NsrR | NO | norB, aniA, nsrR, hmpA, ytfE, |
| | | ygbA, hcp, hcr, nrfA, aox |
| NorR | NO | norVW, norR |
| DNR | NO | norCB, nir, nor, nos |

In some embodiments, the genetically engineered bacteria of the invention comprise a tunable regulatory region that is directly or indirectly controlled by a transcription factor that is capable of sensing at least one reactive nitrogen species. The tunable regulatory region is operatively linked to a gene or genes capable of directly or indirectly driving the expression of an amino acid catabolism enzyme, BCAA transporter, and/or BCAA binding protein, thus controlling expression of the branched chain amino acid catabolism enzyme, BCAA transporter, and/or BCAA binding protein relative to RNS levels. For example, the tunable regulatory region is a RNS-inducible regulatory region, and the payload is an amino acid catabolism enzyme, BCAA transporter, and/or BCAA binding protein, such as any of the amino acid catabolism enzymes, BCAA transporters, and BCAA binding proteins provided herein; when RNS is present, e.g., in an inflamed tissue, a RNS-sensing transcription factor binds to and/or activates the regulatory region and drives expression of the branched chain amino acid catabolism enzyme, BCAA transporter, and/or BCAA binding protein gene or genes. Subsequently, when inflammation is ameliorated, RNS levels are reduced, and production of the branched chain amino acid catabolism enzyme, BCAA transporter, and/or BCAA binding protein is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or genes. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The genetically engineered bacteria of the invention may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012; Table 1). In certain embodiments, the genetically engineered bacteria of the invention comprise a RNS-inducible regulatory region from norVW that is operatively linked to a gene or genes, e.g., one or more branched chain amino acid catabolism enzyme, BCAA transporter, and/or BCAA binding protein gene sequence(s). In the presence of RNS, a NorR transcription factor senses RNS and activates to the norVW regulatory region, thereby driving expression of the operatively linked gene(s) and producing the amino acid catabolism enzyme, BCAA transporter, and/or BCAA binding protein.

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The genetically engineered bacteria of the invention may comprise any suitable RNS-responsive regulatory region from a gene that is activated by DNR. Genes that are capable of being activated by DNR are known in the art (see, e.g., Castiglione et al., 2009; Giardina et al., 2008; Table 1). In certain embodiments, the genetically engineered bacteria of the invention comprise a RNS-inducible regulatory region from norCB that is operatively linked to a gene or gene cassette, e.g., a butyrogenic gene cassette. In the presence of RNS, a DNR transcription factor senses RNS and activates to the norCB regulatory region, thereby driving expression of the operatively linked gene or genes and producing one or more amino acid catabolism enzymes. In some embodiments, the DNR is Pseudomonas aeruginosa DNR.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor [that] can sense NO and control the expression of genes responsible for NO metabolism” (Isabella et al., 2009). The genetically engineered bacteria of the invention may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010; Table 1). In certain embodiments, the genetically engineered bacteria of the invention comprise a RNS-derepressible regulatory region from norB that is operatively linked to a gene or genes, e.g., a branched chain amino acid catabolism enzyme gene or genes. In the presence of RNS, an NsrR transcription factor senses RNS and no longer binds to the norB regulatory region, thereby derepressing the operatively linked branched chain amino acid catabolism enzyme, BCAA transporter, and/or BCAA binding protein gene or genes and producing the encoding an amino acid catabolism enzyme(s).

In some embodiments, it is advantageous for the genetically engineered bacteria to express a RNS-sensing transcription factor that does not regulate the expression of a significant number of native genes in the bacteria. In some embodiments, the genetically engineered bacterium of the invention expresses a RNS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the genetically engineered bacteria.

In some embodiments, the tunable regulatory region is a RNS-repressible regulatory region, and binding of a corresponding transcription factor represses downstream gene expression; in the presence of RNS, the transcription factor senses RNS and binds to the RNS-repressible regulatory region, thereby repressing expression of the operatively linked gene or gene cassette. In some embodiments, the RNS-sensing transcription factor is capable of binding to a regulatory region that overlaps with part of the promoter sequence. In alternate embodiments, the RNS-sensing transcription factor is capable of binding to a regulatory region that is upstream or downstream of the promoter sequence.

In these embodiments, the genetically engineered bacteria may comprise a two repressor activation regulatory circuit, which is used to express an amino acid catabolism enzyme. The two repressor activation regulatory circuit comprises a first RNS-sensing repressor and a second repressor, which is operatively linked to a gene or gene cassette, e.g., encoding an amino acid catabolism enzyme. In one aspect of these embodiments, the RNS-sensing repressor inhibits transcription of the second repressor, which inhibits the transcription of the gene or gene cassette. Examples of second repressors useful in these embodiments include, but are not limited to, TetR, C1, and LexA. In the absence of binding by the first repressor (which occurs in the absence of RNS), the second repressor is transcribed, which represses expression of the gene or genes. In the presence of binding by the first repressor (which occurs in the presence of RNS), expression of the second repressor is repressed, and the gene or genes, e.g., a branched chain amino acid catabolism enzyme, BCAA transporter, and/or BCAA binding protein gene or genes is expressed.

A RNS-responsive transcription factor may induce, derepress, or repress gene expression depending upon the regulatory region sequence used in the genetically engineered bacteria. One or more types of RNS-sensing transcription factors and corresponding regulatory region sequences may be present in genetically engineered bacteria. In some embodiments, the genetically engineered bacteria comprise one type of RNS-sensing transcription factor, e.g., NsrR, and one corresponding regulatory region sequence, e.g., from norB. In some embodiments, the genetically engineered bacteria comprise one type of RNS-sensing transcription factor, e.g., NsrR, and two or more different corresponding regulatory region sequences, e.g., from norB and aniA. In some embodiments, the genetically engineered bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the genetically engineered bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the genetically engineered bacteria of the invention comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of the therapeutic molecule. In some embodiments, expression of the RNS-sensing transcription factor is controlled by the same promoter that controls expression of the therapeutic molecule. In some embodiments, the RNS-sensing transcription factor and therapeutic molecule are divergently transcribed from a promoter region.

In some embodiments, the genetically engineered bacteria of the invention comprise a gene for a RNS-sensing transcription factor from a different species, strain, or substrain of bacteria. In some embodiments, the genetically engineered bacteria comprise a RNS-responsive regulatory region from a different species, strain, or substrain of bacteria. In some embodiments, the genetically engineered bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the genetically engineered bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the genetically engineered bacteria of the invention comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing the therapeutic molecule are present on different plasmids. In some embodiments, the gene encoding the RNS-sensing transcription factor and the gene or gene cassette for producing the therapeutic molecule are present on the same plasmid. In some embodiments, the gene encoding the RNS-sensing transcription factor is present on a chromosome. In some embodiments, the gene encoding the RNS-sensing transcription factor and the gene or gene cassette for producing the therapeutic molecule are present on different chromosomes. In some embodiments, the gene encoding the RNS-sensing transcription factor and the gene or gene cassette for producing the therapeutic molecule are present on the same chromosome.

In some embodiments, the genetically engineered bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the branched chain amino acid catabolism enzyme in the presence of RNS, as compared to the wild-type regulatory region under the same conditions. In some embodiments, the genetically engineered bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the branched chain amino acid catabolism enzyme in the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of the branched chain amino acid catabolism enzyme, BCAA transporter, and/or BCAA binding protein in the presence of RNS.

In some embodiments, the gene or gene cassette for producing the anti-inflammation and/or gut barrier function enhancer molecule is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of a branched chain amino acid catabolism enzyme, BCAA transporter, and/or BCAA binding protein gene(s) may be integrated into the bacterial chromosome. Having multiple copies of the gene or gen(s) integrated into the chromosome allows for greater production of the amino acid catabolism enzyme(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

ROS-Dependent Regulation

In some embodiments, the genetically engineered bacteria comprise a gene for producing an branched chain amino acid catabolism enzyme, BCAA transporter, and/or BCAA binding protein that is expressed under the control of an inducible promoter. In some embodiments, the genetically engineered bacterium that expresses a branched chain amino acid catabolism enzyme, BCAA transporter, and/or BCAA binding protein under the control of a promoter that is activated by conditions of cellular damage. In one embodiment, the gene for producing the branched chain amino acid catabolism enzyme is expressed under the control of a cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H2O2), organic peroxide (ROOH), hydroxyl ion (OH—), hydroxyl radical (.OH), superoxide or superoxide anion (.O2-), singlet oxygen (1O2), ozone (O3), carbonate radical, peroxide or peroxyl radical (.O2-2), hypochlorous acid (HOCl), hypochlorite ion (OCl—), sodium hypochlorite (NaOCl), nitric oxide (NO.), and peroxynitrite or peroxynitrite anion (ONOO—) (unpaired electrons denoted by .). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more amino acid catabolism enzyme(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or genes.

As used herein, “ROS-derepressible regulatory region” refers to a nucleic acid sequence to which one or more ROS-sensing transcription factors is capable of binding, wherein the binding of the corresponding transcription factor represses downstream gene expression; in the presence of ROS, the transcription factor does not bind to and does not repress the regulatory region. In some embodiments, the ROS-derepressible regulatory region comprises a promoter sequence. The ROS-derepressible regulatory region may be operatively linked to a gene or genes, e.g., one or more genes encoding one or more amino acid catabolism enzyme(s). For example, in the presence of ROS, a transcription factor, e.g., OhrR, senses ROS and no longer binds to and/or represses the regulatory region, thereby derepressing an operatively linked gene sequence or gene cassette. Thus, ROS derepresses expression of the gene or gene cassette.

As used herein, “ROS-repressible regulatory region” refers to a nucleic acid sequence to which one or more ROS-sensing transcription factors is capable of binding, wherein the binding of the corresponding transcription factor represses downstream gene expression; in the presence of ROS, the transcription factor binds to and represses the regulatory region. In some embodiments, the ROS-repressible regulatory region comprises a promoter sequence. In some embodiments, the transcription factor that senses ROS is capable of binding to a regulatory region that overlaps with part of the promoter sequence. In alternate embodiments, the transcription factor that senses ROS is capable of binding to a regulatory region that is upstream or downstream of the promoter sequence. The ROS-repressible regulatory region may be operatively linked to a gene sequence or gene sequences. For example, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or genes.

As used herein, a “ROS-responsive regulatory region” refers to a ROS-inducible regulatory region, a ROS-repressible regulatory region, and/or a ROS-derepressible regulatory region. In some embodiments, the ROS-responsive regulatory region comprises a promoter sequence. Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 7.

TABLE 7
Examples of ROS-sensing transcription
factors and ROS-responsive genes

| ROS-sensing | | |
|---|---|---|
| transcription | Primarily capable | Examples of responsive genes, |
| factor: | of sensing: | promoters, and/or regulatory regions: |
| OxyR | H₂O₂ | ahpC; ahpF; dps; dsbG; fhuF; flu; fur; |
| | | gor; grxA; hemH; katG; oxyS; sufA; |
| | | sufB; sufC; sufD; sufE; sufS; trxC; |
| | | uxuA; yaaA; yaeH; yaiA; ybjM; ydcH; |
| | | ydeN; ygaQ; yljA; ytfK |
| PerR | H₂O₂ | katA; ahpCF; mrgA; zoaA; fur; |
| | | hemAXCDBL; srfA |
| OhrR | Organic peroxides | ohrA |
| | NaOCl | |
| SoxR | •O₂⁻ | soxS |
| | NO• | |
| | (also capable of | |
| | sensing H₂O₂) | |
| RosR | H₂O₂ | rbtT; tnp16a; rluC1; tnp5a; mscL; |
| | | tnp2d; phoD; tnp15b; pstA; tnp5b; |
| | | xylC; gabD1; rluC2; cgtS9; azlC; |
| | | narKGHJI; rosR |

In some embodiments, the genetically engineered bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of an amino acid catabolism enzyme, thus controlling expression of the branched chain amino acid catabolism enzymerelative to ROS levels. For example, the tunable regulatory region is a ROS-inducible regulatory region, and the molecule is an amino acid catabolism enzyme; when ROS is present, e.g., in an inflamed tissue, a ROS-sensing transcription factor binds to and/or activates the regulatory region and drives expression of the gene sequence for the amino acid catabolism enzyme, BCAA transporter, and/or BCAA binding protein thereby producing the amino acid catabolism enzyme, BCAA transporter, and/or BCAA binding protein. Subsequently, when inflammation is ameliorated, ROS levels are reduced, and production of the branched chain amino acid catabolism enzyme, BCAA transporter, and/or BCAA binding protein is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H2O2 detoxification (katE, ahpCF), heme biosynthesis (hemH), reductant supply (grxA, gor, trxC), thiol-disulfide isomerization (dsbG), Fe—S center repair (sufA-E, sufS), iron binding (yaaA), repression of iron import systems (fur)” and “OxyS, a small regulatory RNA” (Dubbs et al., 2012). The genetically engineered bacteria may comprise any suitable ROS-responsive regulatory region from a gene that is activated by OxyR. Genes that are capable of being activated by OxyR are known in the art (see, e.g., Zheng et al., 2001; Dubbs et al., 2012; Table 1). In certain embodiments, the genetically engineered bacteria of the invention comprise a ROS-inducible regulatory region from oxyS that is operatively linked to a gene, e.g., a branched chain amino acid catabolism enzyme, BCAA transporter, and/or BCAA binding protein gene. In the presence of ROS, e.g., H2O2, an OxyR transcription factor senses ROS and activates to the oxyS regulatory region, thereby driving expression of the operatively linked branched chain amino acid catabolism enzyme, BCAA transporter, and/or BCAA binding protein gene and producing the amino acid catabolism enzyme, BCAA transporter, and/or BCAA binding protein. In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H2O2. The genetically engineered bacteria of the invention may comprise any suitable ROS-responsive regulatory region from a gene that is activated by SoxR. Genes that are capable of being activated by SoxR are known in the art (see, e.g., Koo et al., 2003; Table 1). In certain embodiments, the genetically engineered bacteria of the invention comprise a ROS-inducible regulatory region from soxS that is operatively linked to a gene, e.g., an amino acid catabolism enzyme. In the presence of ROS, the SoxR transcription factor senses ROS and activates the soxS regulatory region, thereby driving expression of the operatively linked branched chain amino acid catabolism enzyme, BCAA transporter, and/or BCAA binding protein gene and producing an amino acid catabolism enzyme, BCAA transporter, and/or BCAA binding protein.

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that] . . . senses both organic peroxides and NaOCl” (Dubbs et al., 2012) and is “weakly activated by H2O2 but it shows much higher reactivity for organic hydroperoxides” (Duarte et al., 2010). The genetically engineered bacteria of the invention may comprise any suitable ROS-responsive regulatory region from a gene that is repressed by OhrR. Genes that are capable of being repressed by OhrR are known in the art (see, e.g., Dubbs et al., 2012; Table 1). In certain embodiments, the genetically engineered bacteria of the invention comprise a ROS-derepressible regulatory region from ohrA that is operatively linked to a gene or gene cassette, e.g., a branched chain amino acid catabolism enzyme, BCAA transporter, and/or BCAA binding protein gene. In the presence of ROS, e.g., NaOCl, an OhrR transcription factor senses ROS and no longer binds to the ohrA regulatory region, thereby derepressing the operatively linked branched chain amino acid catabolism enzyme, BCAA transporter, and/or BCAA binding protein gene and producing an amino acid catabolism enzyme, BCAA transporter, and/or BCAA binding protein.

OhrR is a member of the MarR family of ROS-responsive regulators. “Most members of the MarR family are transcriptional repressors and often bind to the −10 or −35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the genetically engineered bacteria of the invention comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA” (SEQ ID NO: 144) and is “reversibly inhibited by the oxidant H2O2” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The genetically engineered bacteria of the invention may comprise any suitable ROS-responsive regulatory region from a gene that is repressed by RosR. Genes that are capable of being repressed by RosR are known in the art (see, e.g., Bussmann et al., 2010; Table 1). In certain embodiments, the genetically engineered bacteria of the invention comprise a ROS-derepressible regulatory region from cgtS9 that is operatively linked to a gene or gene cassette, e.g., an amino acid catabolism enzyme, BCAA transporter, and/or BCAA binding protein. In the presence of ROS, e.g., H2O2, a RosR transcription factor senses ROS and no longer binds to the cgtS9 regulatory region, thereby derepressing the operatively linked branched chain amino acid catabolism enzyme, BCAA transporter, and/or BCAA binding protein gene and producing the amino acid catabolism enzyme, BCAA transporter, and/or BCAA binding protein.

In some embodiments, it is advantageous for the genetically engineered bacteria to express a ROS-sensing transcription factor that does not regulate the expression of a significant number of native genes in the bacteria. In some embodiments, the genetically engineered bacterium of the invention expresses a ROS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the genetically engineered bacteria.

In some embodiments, the tunable regulatory region is a ROS-repressible regulatory region, and binding of a corresponding transcription factor represses downstream gene expression; in the presence of ROS, the transcription factor senses ROS and binds to the ROS-repressible regulatory region, thereby repressing expression of the operatively linked gene or gene cassette. In some embodiments, the ROS-sensing transcription factor is capable of binding to a regulatory region that overlaps with part of the promoter sequence. In alternate embodiments, the ROS-sensing transcription factor is capable of binding to a regulatory region that is upstream or downstream of the promoter sequence.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA)(SEQ ID NO: 145) residing within and near the promoter sequences of PerR-controlled genes” (Marinho et al., 2014). PerR is capable of binding a regulatory region that “overlaps part of the promoter or is immediately downstream from it” (Dubbs et al., 2012). The genetically engineered bacteria of the invention may comprise any suitable ROS-responsive regulatory region from a gene that is repressed by PerR. Genes that are capable of being repressed by PerR are known in the art (see, e.g., Dubbs et al., 2012; Table 1).

In these embodiments, the genetically engineered bacteria may comprise a two repressor activation regulatory circuit, which is used to express an amino acid catabolism enzyme. The two repressor activation regulatory circuit comprises a first ROS-sensing repressor, e.g., PerR, and a second repressor, e.g., TetR, which is operatively linked to a gene or gene cassette, e.g., an amino acid catabolism enzyme. In one aspect of these embodiments, the ROS-sensing repressor inhibits transcription of the second repressor, which inhibits the transcription of the gene or gene cassette. Examples of second repressors useful in these embodiments include, but are not limited to, TetR, C1, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., an amino acid catabolism enzyme. In the absence of PerR binding (which occurs in the absence of ROS), tetR is transcribed, and TetR represses expression of the gene or gene cassette, e.g., an amino acid catabolism enzyme. In the presence of PerR binding (which occurs in the presence of ROS), tetR expression is repressed, and the gene or gene cassette, e.g., an amino acid catabolism enzyme, BCAA transporter, and/or BCAA binding protein is expressed.

A ROS-responsive transcription factor may induce, derepress, or repress gene expression depending upon the regulatory region sequence used in the genetically engineered bacteria. For example, although “OxyR is primarily thought of as a transcriptional activator under oxidizing conditions . . . OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The genetically engineered bacteria of the invention may comprise any suitable ROS-responsive regulatory region from a gene that is repressed by OxyR. In some embodiments, OxyR is used in a two repressor activation regulatory circuit, as described above. Genes that are capable of being repressed by OxyR are known in the art (see, e.g., Zheng et al., 2001; Table 1). Or, for example, although RosR is capable of repressing a number of genes, it is also capable of activating certain genes, e.g., the narKGHJI operon. In some embodiments, the genetically engineered bacteria comprise any suitable ROS-responsive regulatory region from a gene that is activated by RosR. In addition, “PerR-mediated positive regulation has also been observed . . . and appears to involve PerR binding to distant upstream sites” (Dubbs et al., 2012). In some embodiments, the genetically engineered bacteria comprise any suitable ROS-responsive regulatory region from a gene that is activated by PerR.

One or more types of ROS-sensing transcription factors and corresponding regulatory region sequences may be present in genetically engineered bacteria. For example, “OhrR is found in both Gram-positive and Gram-negative bacteria and can coreside with either OxyR or PerR or both” (Dubbs et al., 2012). In some embodiments, the genetically engineered bacteria comprise one type of ROS-sensing transcription factor, e.g., OxyR, and one corresponding regulatory region sequence, e.g., from oxyS. In some embodiments, the genetically engineered bacteria comprise one type of ROS-sensing transcription factor, e.g., OxyR, and two or more different corresponding regulatory region sequences, e.g., from oxyS and katG. In some embodiments, the genetically engineered bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the genetically engineered bacteria comprise two or more types of ROS-sensing transcription factors and one corresponding regulatory region sequence.

Nucleic acid sequences of several exemplary OxyR-regulated regulatory regions are shown in Table 5. OxyR binding sites are underlined and bolded. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 46, 47, 48, or 49, or a functional fragment thereof.

TABLE 8
Nucleotide sequences of exemplary OxyR-regulated regulatory regions

| Regulatory | |
|---|---|
| sequence | 01234567890123456789012345678901234567890123456789 |
| katG | TGTGGCTTTTATGAAAATCACACAGTGATCACAAATTTTAAACA |
| (SEQ ID NO: | GAGCACAAAATGCTGCCTCGAAATGAGGGCGGGAAAATAAGGT |
| 46) | TATCAGCCTTGTTTTCTCCCTCATTACTTGAAGGATATGAAGCTA |
| | AAACCCTTTTTTATAAAGCATTTGTCCGAATTCGGACATAATCA |
| | AAAAAGCTTAATTAAGATCAATTTGATCTACATCTCTTTAACCA |
| | ACAATATGTAAGATCTCAACTATCGCATCCGTGGATTAATTC |
| | AATTATAACTTCTCTCTAACGCTGTGTATCGTAACGGTAACACT |
| | GTAGAGGGGAGCACATTGATGCGAATTCATTAAAGAGGAGAAA |
| | GGTACC |
| dps | TTCCGAAAATTCCTGGCGAGCAGATAAATAAGAATTGTTCTTAT |
| (SEQ ID NO: | CAATATATCTAACTCATTGAATCTTTATTAGTTTTGTTTTTCACG |
| 47) | CTTGTTACCACTATTAGTGTGATAGGAACAGCCAGAATAGCG |
| | GAACACATAGCCGGTGCTATACTTAATCTCGTTAATTACTGGGA |
| | CATAACATCAAGAGGATATGAAATTCGAATTCATTAAAGAGGA |
| | GAAAGGTACC |
| ahpC | GCTTAGATCAGGTGATTGCCCTTTGTTTATGAGGGTGTTGTAATC |
| (SEQ ID NO: | CATGTCGTTGTTGCATTTGTAAGGGCAACACCTCAGCCTGCAGG |
| 48) | CAGGCACTGAAGATACCAAAGGGTAGTTCAGATTACACGGTCA |
| | CCTGGAAAGGGGGCCATTTTACTTTTTATCGCCGCTGGCGGTGC |
| | AAAGTTCACAAAGTTGTCTTACGAAGGTTGTAAGGTAAAACTT |
| | ATCGATTTGATAATGGAAACGCATTAGCCGAATCGGCAAAAAT |
| | TGGTTACCTTACATCTCATCGAAAACACGGAGGAAGTATAGATG |
| | CGAATTCATTAAAGAGGAGAAAGGTACC |
| oxyS | CTCGAGTTCATTATCCATCCTCCATCGCCACGATAGTTCATGGC |
| (SEQ ID NO: | GATAGGTAGAATAGCAATGAACGATTATCCCTATCAAGCATTC |
| 49) | TGACTGATAATTGCTCACACGAATTCATTAAAGAGGAGAAAGGT |
| | ACC |

In some embodiments, the genetically engineered bacteria of the invention comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

In some embodiments, the genetically engineered bacteria of the invention comprise a gene for a ROS-sensing transcription factor from a different species, strain, or substrain of bacteria. In some embodiments, the genetically engineered bacteria comprise a ROS-responsive regulatory region from a different species, strain, or substrain of bacteria. In some embodiments, the genetically engineered bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the genetically engineered bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the genetically engineered bacteria of the invention comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing the therapeutic molecule are present on different plasmids. In some embodiments, the gene encoding the ROS-sensing transcription factor and the gene or gene cassette for producing the therapeutic molecule are present on the same. In some embodiments, the gene encoding the ROS-sensing transcription factor is present on a chromosome. In some embodiments, the gene encoding the ROS-sensing transcription factor and the gene or gene cassette for producing the therapeutic molecule are present on different chromosomes. In some embodiments, the gene encoding the ROS-sensing transcription factor and the gene or gene cassette for producing the therapeutic molecule are present on the same chromosome.

In some embodiments, the genetically engineered bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the branched chain amino acid catabolism enzyme, BCAA transporter, and/or BCAA binding protein in the presence of ROS, as compared to the wild-type regulatory region under the same conditions. In some embodiments, the genetically engineered bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the branched chain amino acid catabolism enzyme, BCAA transporter, and/or BCAA binding protein in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the branched chain amino acid catabolism enzyme in the presence of ROS.

In some embodiments, the gene or gene cassette for producing the branched chain amino acid catabolism enzyme is present on a plasmid and operably linked to a promoter that is induced by ROS. In some embodiments, the gene or gene cassette for producing the branched chain amino acid catabolism enzyme is present in the chromosome and operably linked to a promoter that is induced by ROS. In some embodiments, the gene or gene cassette for producing the branched chain amino acid catabolism enzyme is present on a chromosome and operably linked to a promoter that is induced by exposure to tetracycline. In some embodiments, the gene or gene cassette for producing the branched chain amino acid catabolism enzyme, BCAA transporter, and/or BCAA binding protein is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the genetically engineered bacteria may comprise multiple copies of the gene(s) capable of producing an amino acid catabolism enzyme(s), BCAA transporter(s), and/or BCAA binding protein(s). In some embodiments, the gene(s) capable of producing an amino acid catabolism enzyme(s), BCAA transporter(s), and/or BCAA binding protein(s) is present on a plasmid and operatively linked to a ROS-responsive regulatory region. In some embodiments, the gene(s) capable of producing a branched chain amino acid catabolism enzyme, BCAA transporter, and/or BCAA binding protein is present in a chromosome and operatively linked to a ROS-responsive regulatory region.

Thus, in some embodiments, the genetically engineered bacteria or genetically engineered yeast or virus produce one or more amino acid catabolism enzymes under the control of an oxygen level-dependent promoter, a reactive oxygen species (ROS)-dependent promoter, or a reactive nitrogen species (RNS)-dependent promoter, and a corresponding transcription factor.

In some embodiments, the genetically engineered bacteria comprise a stably maintained plasmid or chromosome carrying a gene for producing an amino acid catabolism enzyme, BCAA transporter, and/or BCAA binding protein such that the branched chain amino acid catabolism enzyme, BCAA transporter, and/or BCAA binding protein can be expressed in the host cell, and the host cell is capable of survival and/or growth in vitro, e.g., in medium, and/or in vivo. In some embodiments, a bacterium may comprise multiple copies of the gene encoding the amino acid catabolism enzyme, BCAA transporter, and/or BCAA binding protein. In some embodiments, the gene encoding the branched chain amino acid catabolism enzyme, BCAA transporter, and/or BCAA binding protein is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the branched chain amino acid catabolism enzyme, BCAA transporter, and/or BCAA binding protein is expressed on a high-copy plasmid. In some embodiments, the high-copy plasmid may be useful for increasing expression of the amino acid catabolism enzyme, BCAA transporter, and/or BCAA binding protein. In some embodiments, the gene encoding the branched chain amino acid catabolism enzyme, BCAA transporter, and/or BCAA binding protein is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene encoding a particular branched chain amino acid catabolism enzyme, BCAA transporter, and/or BCAA binding protein inserted at four different insertion sites. Alternatively, the genetically engineered bacteria may include three copies of the gene encoding a particular branched chain amino acid catabolism enzyme, BCAA transporter, and/or BCAA binding protein inserted at three different insertion sites and three copies of the gene encoding a different branched chain amino acid catabolism enzyme, BCAA transporter, and/or BCAA binding protein inserted at three different insertion sites.

In some embodiments, under conditions where the branched chain amino acid catabolism enzyme, BCAA transporter, and/or BCAA binding protein is expressed, the genetically engineered bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the amino acid catabolism enzyme, BCAA transporter, and/or BCAA binding protein and/or transcript of the gene(s) in the operon as compared to unmodified bacteria of the same subtype under the same conditions.

In some embodiments, quantitative PCR (qPCR) is used to amplify, detect, and/or quantify mRNA expression levels of the branched chain amino acid catabolism enzyme, BCAA transporter, and/or BCAA binding protein gene(s). Primers specific for branched chain amino acid catabolism enzyme, BCAA transporter, and/or BCAA binding protein gene(s) may be designed and used to detect mRNA in a sample according to methods known in the art. In some embodiments, a fluorophore is added to a sample reaction mixture that may contain branched chain amino acid catabolism enzymemRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the branched chain amino acid catabolism enzyme, BCAA transporter, and/or BCAA binding protein gene(s).

In some embodiments, quantitative PCR (qPCR) is used to amplify, detect, and/or quantify mRNA expression levels of the branched chain amino acid catabolism enzyme, BCAA transporter, and/or BCAA binding protein gene(s). Primers specific for branched chain amino acid catabolism enzyme, BCAA transporter, and/or BCAA binding protein gene(s) may be designed and used to detect mRNA in a sample according to methods known in the art. In some embodiments, a fluorophore is added to a sample reaction mixture that may contain branched chain amino acid catabolism enzyme, BCAA transporter, and/or BCAA binding protein mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the branched chain amino acid catabolism enzyme, BCAA transporter, and/or BCAA binding protein gene(s).

In other embodiments, the inducible promoter is a propionate responsive promoter. For example, the prpR promoter is a propionate responsive promoter. In one embodiment, the propionate responsive promoter comprises SEQ ID NO: 13.

Inducible Promoters (Nutritional and/or Chemical Inducer(s) and/or Metabolite(s))

In some embodiments, one or more gene sequence(s) encoding the branched chain amino acid catabolism enzyme(s) e.g., kivD, leuDH, ilvE, L-AAD, BCKD, adh2, PadA, and/or YqhD, is present on a plasmid and operably linked to promoter a directly or indirectly inducible by one or more nutritional and/or chemical inducer(s) and/or metabolite(s). In some embodiments, the bacterial cell comprises a stably maintained plasmid or chromosome carrying the gene encoding the branched chain amino acid catabolism enzyme, which is induced by one or more nutritional and/or chemical inducer(s) and/or metabolite(s), such that the branched chain amino acid catabolism enzyme can be expressed in the host cell, and the host cell is capable of survival and/or growth in vitro, e.g., under culture conditions, and/or in vivo, e.g., in the gut. In some embodiments, bacterial cell comprises two or more distinct branched chain amino acid catabolism cassette(s), one or more of which are induced by one or more nutritional and/or chemical inducer(s) and/or metabolite(s). In some embodiments, the genetically engineered bacteria comprise multiple copies of the same branched chain amino acid catabolism enzyme gene(s) and/or gene cassette(s) which are induced by one or more nutritional and/or chemical inducer(s) and/or metabolite(s). In some embodiments, the genetically engineered bacteria comprise multiple copies of different branched chain amino acid catabolism enzyme genes or gene cassette(s), one or more of which are induced by one or more nutritional and/or chemical inducer(s) and/or metabolite(s).

In some embodiments, the gene encoding the branched chain amino acid catabolism enzyme is present on a plasmid and operably linked to a promoter that is induced by one or more nutritional and/or chemical inducer(s) and/or metabolite(s). In some embodiments, the gene encoding the branched chain amino acid catabolism enzyme is present in the chromosome and operably linked to a promoter that is induced by one or more nutritional and/or chemical inducer(s) and/or metabolite(s).

In some embodiments, the bacterial cell comprises a stably maintained plasmid or chromosome carrying the one or more gene sequences(s), inducible by one or more nutritional and/or chemical inducer(s) and/or metabolite(s), encoding a transporter of branched chain amino acid(s) and/or one or more metabolites thereof, e.g., livKHMGF and/or brnQ, such that the transporter can be expressed in the host cell, and the host cell is capable of survival and/or growth in vitro, e.g., in medium, and/or in vivo, e.g., in the gut. In some embodiments, bacterial cell comprises two or more distinct copies of the one or more gene sequences(s) encoding a branched chain amino acid transporter, which is controlled by a promoter inducible one or more nutritional and/or chemical inducer(s) and/or metabolite(s). In some embodiments, the genetically engineered bacteria comprise multiple copies of the same one or more gene sequences(s) encoding a branched chain amino acid transporter, which is controlled by a promoter inducible one or more nutritional and/or chemical inducer(s) and/or metabolite(s). In some embodiments, the one or more gene sequences(s) encoding a transporter of branched chain amino acid(s), is present on a plasmid and operably linked to a directly or indirectly inducible promoter inducible by one or more nutritional and/or chemical inducer(s) and/or metabolite(s). In some embodiments, the one or more gene sequences(s) encoding a branched chain amino acid transporter, is present on a chromosome and operably linked to a directly or indirectly inducible by one or more nutritional and/or chemical inducer(s) and/or metabolite(s).

In some embodiments, one or more gene sequence(s) encoding branched chain amino acid binding protein(s), e.g., ilvJ, is present on a plasmid and operably linked to promoter a directly or indirectly inducible by one or more nutritional and/or chemical inducer(s) and/or metabolite(s). In some embodiments, the bacterial cell comprises a stably maintained plasmid or chromosome carrying the gene encoding branched chain amino acid binding protein, which is induced by one or more nutritional and/or chemical inducer(s) and/or metabolite(s), such that branched chain amino acid binding protein can be expressed in the host cell, and the host cell is capable of survival and/or growth in vitro, e.g., under culture conditions, and/or in vivo, e.g., in the gut. In some embodiments, bacterial cell comprises two or more distinct branched chain amino acid catabolism cassette(s), one or more of which are induced by one or more nutritional and/or chemical inducer(s) and/or metabolite(s). In some embodiments, the genetically engineered bacteria comprise multiple copies of the same branched chain amino acid catabolism enzyme gene(s) and/or gene cassette(s) which are induced by one or more nutritional and/or chemical inducer(s) and/or metabolite(s). In some embodiments, the genetically engineered bacteria comprise multiple copies of different branched chain amino acid catabolism enzyme genes or gene cassette(s), one or more of which are induced by one or more nutritional and/or chemical inducer(s) and/or metabolite(s).

In some embodiments, the gene encoding branched chain amino acid binding protein is present on a plasmid and operably linked to a promoter that is induced by one or more nutritional and/or chemical inducer(s) and/or metabolite(s). In some embodiments, the gene encoding branched chain amino acid binding protein is present in the chromosome and operably linked to a promoter that is induced by one or more nutritional and/or chemical inducer(s) and/or metabolite(s).

In some embodiments, one or more gene sequence(s) encoding branched chain amino acid exporter(s), e.g., ilvJ, is present on a plasmid and operably linked to promoter a directly or indirectly inducible by one or more nutritional and/or chemical inducer(s) and/or metabolite(s). In some embodiments, the bacterial cell comprises a stably maintained plasmid or chromosome carrying the gene encoding branched chain amino acid exporter, which is induced by one or more nutritional and/or chemical inducer(s) and/or metabolite(s), such that branched chain amino acid exporter can be expressed in the host cell, and the host cell is capable of survival and/or growth in vitro, e.g., under culture conditions, and/or in vivo, e.g., in the gut. In some embodiments, bacterial cell comprises two or more distinct branched chain amino acid catabolism cassette(s), one or more of which are induced by one or more nutritional and/or chemical inducer(s) and/or metabolite(s). In some embodiments, the genetically engineered bacteria comprise multiple copies of the same branched chain amino acid catabolism enzyme gene(s) and/or gene cassette(s) which are induced by one or more nutritional and/or chemical inducer(s) and/or metabolite(s). In some embodiments, the genetically engineered bacteria comprise multiple copies of different branched chain amino acid catabolism enzyme genes or gene cassette(s), one or more of which are induced by one or more nutritional and/or chemical inducer(s) and/or metabolite(s).

In some embodiments, the gene encoding branched chain amino acid exporter is present on a plasmid and operably linked to a promoter that is induced by one or more nutritional and/or chemical inducer(s) and/or metabolite(s). In some embodiments, the gene encoding branched chain amino acid exporter is present in the chromosome and operably linked to a promoter that is induced by one or more nutritional and/or chemical inducer(s) and/or metabolite(s).

In some embodiments, the promoter that is operably linked to the gene encoding the branched chain amino acid catabolism enzyme and the promoter that is operably linked to the gene encoding the branched chain amino acid transporter and/or BCAA binding protein and/or BCAA exporter, is directly or indirectly induced by one or more nutritional and/or chemical inducer(s) and/or metabolite(s).

In some embodiments, one or more inducible promoter(s) are useful for or induced during in vivo expression of the one or more protein(s) of interest. In some embodiments, the promoters are induced during in vivo expression of one or more branched chain amino acid catabolism enzymes and/or branched chain amino acid transporter(s) and/or BCAA binding protein(s) and/or BCAA exporter(s). In some embodiments, expression of one or more branched chain amino acid catabolism enzyme(s) and/or branched chain amino acid transporter(s) and/or BCAA binding protein(s) and/or BCAA exporter(s) is driven directly or indirectly by one or more arabinose inducible promoter(s) in vivo. In some embodiments, the promoter is directly or indirectly induced by a chemical and/or nutritional inducer and/or metabolite which is co-administered with the genetically engineered bacteria of the invention.

In some embodiments, expression of one or more branched chain amino acid catabolism enzyme gene(s) and/or branched chain amino acid transporter(s) and/or BCAA binding protein(s) and/or BCAA exporter(s), is driven directly or indirectly by one or more promoter(s) induced by a chemical and/or nutritional inducer and/or metabolite during in vitro growth, preparation, or manufacturing of the strain prior to in vivo administration. In some embodiments, the promoter(s) induced by a chemical and/or nutritional inducer and/or metabolite are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with branched chain amino acid catabolism enzyme(s) and/or branched chain amino acid transporter(s) and/or BCAA binding protein(s) and/or BCAA exporter(s) prior to administration. In some embodiments, the cultures, which are induced by a chemical and/or nutritional inducer and/or metabolite, are grown aerobically. In some embodiments, the cultures, which are induced by a chemical and/or nutritional inducer and/or metabolite, are grown anaerobically.

In some embodiments, the genetically engineered bacteria encode one or more gene sequence(s) which are inducible through an arabinose inducible system.

The genes of arabinose metabolism are organized in one operon, AraBAD, which is controlled by the PAraBAD promoter. The PAraBAD (or Para) promoter suitably fulfills the criteria of inducible expression systems. PAraBAD displays tighter control of payload gene expression than many other systems, likely due to the dual regulatory role of AraC, which functions both as an inducer and as a repressor. Additionally, the level of ParaBAD-based expression can be modulated over a wide range of L-arabinose concentrations to fine-tune levels of expression of the payload. However, the cell population exposed to sub-saturating L-arabinose concentrations is divided into two subpopulations of induced and uninduced cells, which is determined by the differences between individual cells in the availability of L-arabinose transporter (Zhang et al., Development and Application of an Arabinose-Inducible Expression System by Facilitating Inducer Uptake in Corynebacterium glutamicum; Appl. Environ. Microbiol. August 2012 vol. 78 no. 16 5831-5838). Alternatively, inducible expression from the ParaBad can be controlled or fine-tuned through the optimization of the ribosome binding site (RBS), as described herein.

In one embodiment, expression of one or more branched chain amino acid catabolism enzyme(s), e.g., kivD, leuDH, ilvE, L-AAD, BCKD, adh2, PadA, and/or YqhD, e.g., as described herein, is driven directly or indirectly by one or more arabinose inducible promoter(s). In one embodiment, the genetically engineered bacteria encode one or more branched chain amino acid transporter(s) e.g., livKHMGF and/or brnQ, described herein, whose expression is driven directly or indirectly by one or more arabinose inducible promoter(s). In one embodiment, expression of one or more branched chain amino acid binding protein(s), e.g., ilvJ, e.g., as described herein, is driven directly or indirectly by one or more arabinose inducible promoter(s). In one embodiment, expression of one or more branched chain amino acid exporter(s), e.g., as described herein, is driven directly or indirectly by one or more arabinose inducible promoter(s). In some embodiments, the arabinose inducible promoter is useful for or induced during in vivo expression of the one or more protein(s) of interest. In some embodiments, expression of one or more branched chain amino acid catabolism enzyme(s) and/or branched chain amino acid transporter(s) and/or BCAA binding protein(s) and/or BCAA exporter(s) is driven directly or indirectly by one or more arabinose inducible promoter(s) in vivo. In some embodiments, the promoter is directly or indirectly induced by a molecule (e.g., arabinose) that is co-administered with the genetically engineered bacteria of the invention.

In some embodiments, expression of one or more branched chain amino acid catabolism enzyme(s) and/or branched chain amino acid transporter(s) and/or BCAA binding protein(s) and/or BCAA exporter(s), is driven directly or indirectly by one or more arabinose inducible promoter(s) during in vitro growth, preparation, or manufacturing of the strain prior to in vivo administration. In some embodiments, the arabinose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., arabinose, that is added to in the bacterial culture to induce expression and pre-load the bacterium with branched chain amino acid catabolism enzyme(s) and/or branched chain amino acid transporter(s) and/or BCAA binding protein(s) and/or BCAA exporter(s) prior to administration. In some embodiments, the cultures, which are induced by arabinose, are grown aerobically. In some embodiments, the cultures, which are induced by arabinose, are grown anaerobically.

In some embodiments, bacterial cell comprises two or more distinct branched chain amino acid catabolism cassette(s) or transporter(s) or binding protein(s) or exporter(s), one or more of which are induced by arabinose. In some embodiments, the genetically engineered bacteria comprise multiple copies of the same branched chain amino acid catabolism enzyme gene sequence(s) and/or transporter gene sequence(s), e.g., as described herein, which are induced by one or more nutritional and/or chemical inducer(s) and/or metabolite(s). In some embodiments, the genetically engineered bacteria comprise multiple copies of different branched chain amino acid catabolism enzyme genes sequence(s) and/or transporter gene sequence(s) and/or BCAA binding protein gene sequence(s) and/or BCAA exporter gene sequence(s), e.g., as described herein, one or more of which are induced by one or more nutritional and/or chemical inducer(s) and/or metabolite(s).

In a first example, the arabinose inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the arabinose inducible promoter drives expression under a first set of exogenous conditions, and the second promoter drives the expression under a second set of exogenous conditions. In second example, the arabinose promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., arabinose and IPTG). In another non-limiting example, the first inducing conditions are culture conditions, e.g., the presence of arabinose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more arabinose promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by arabinose. In some embodiments, the gene sequence(s) encoding the branched chain amino acid catabolism enzyme(s) or branched chain amino acid transporter(s) is present in the chromosome and operably linked to a promoter that is induced by arabinose.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 103. In some embodiments, the arabinose inducible construct further comprises a gene encoding AraC, which is divergently transcribed from the same promoter as the one or more one or more branched chain amino acid catabolism enzyme(s). In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 104. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 104.

In some embodiments, the genetically engineered bacteria comprise one or more gene sequence(s) which are inducible through a rhamnose inducible system. The genes rhaBAD are organized in one operon which is controlled by the rhaP BAD promoter. The rhaP BAD promoter is regulated by two activators, RhaS and RhaR, and the corresponding genes belong to one transcription unit which divergently transcribed in the opposite direction of rhaBAD. In the presence of L-rhamnose, RhaR binds to the rhaP RS promoter and activates the production of RhaR and RhaS. RhaS together with L-rhamnose then bind to the rhaP BAD and the rhaP T promoter and activate the transcription of the structural genes. In contrast to the arabinose system, in which AraC is provided and divergently transcribed in the gene sequence(s), it is not necessary to express the regulatory proteins in larger quantities in the rhamnose expression system because the amounts expressed from the chromosome are sufficient to activate transcription even on multi-copy plasmids. Therefore, only the rhaP BAD promoter is cloned upstream of the gene that is to be expressed. Full induction of rhaBAD transcription also requires binding of the CRP-cAMP complex, which is a key regulator of catabolite repression. Alternatively, inducible expression from the rhaBAD can be controlled or fine-tuned through the optimization of the ribosome binding site (RBS), as described herein.

In one embodiment, expression of one or more branched chain amino acid catabolism enzyme(s), e.g., kivD, leuDH, ilvE, L-AAD, BCKD, adh2, PadA, and/or YqhD, e.g., as described herein, is driven directly or indirectly by one or more rhamnose inducible promoter(s). In one embodiment, the genetically engineered bacteria encode one or more branched chain amino acid transporter(s), e.g., livKHMGF and/or brnQ, described herein, whose expression is driven directly or indirectly by one or more rhamnose inducible promoter(s).). In one embodiment, the genetically engineered bacteria encode one or more branched chain amino acid binding protein(s), e.g., ilvJ, described herein, whose expression is driven directly or indirectly by one or more rhamnose inducible promoter(s).). In one embodiment, the genetically engineered bacteria encode one or more branched chain amino acid exporter(s), described herein, whose expression is driven directly or indirectly by one or more rhamnose inducible promoter(s).

In some embodiments, the rhamnose inducible promoter is useful for or induced during in vivo expression of the one or more protein(s) of interest. In some embodiments, expression of one or more branched chain amino acid catabolism enzyme(s) and/or branched chain amino acid transporter(s) and/or BCAA binding protein(s) and/or BCAA exporter(s) is driven directly or indirectly by one or more rhamnose inducible promoter(s) in vivo. In some embodiments, the promoter is directly or indirectly induced by a molecule (e.g., rhamnose) that is co-administered with the genetically engineered bacteria of the invention.

In some embodiments, expression of one or more branched chain amino acid catabolism enzyme(s) and/or branched chain amino acid transporter(s) and/or BCAA binding protein(s) and/or BCAA exporter(s), is driven directly or indirectly by one or more rhamnose inducible promoter(s) during in vitro growth, preparation, or manufacturing of the strain prior to in vivo administration. In some embodiments, the rhamnose inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., rhamnose, that is added to in the bacterial culture to induce expression and pre-load the bacterium with branched chain amino acid catabolism enzyme(s) and/or BCAA transporter(s) and/or BCAA binding protein(s) and/or BCAA exporter(s) prior to administration. In some embodiments, the cultures, which are induced by rhamnose, are grown aerobically. In some embodiments, the cultures, which are induced by rhamnose, are grown anaerobically.

In some embodiments, bacterial cell comprises two or more distinct branched chain amino acid catabolism cassette(s) or other polypeptide(s) of interest, one or more of which are induced by rhamnose. In some embodiments, the genetically engineered bacteria comprise multiple copies of the same branched chain amino acid catabolism enzyme gene sequence(s) and/or transporter gene sequence(s) and/or BCAA binding protein gene sequence(s) and/or BCAA exporter gene sequence(s), e.g., as described herein, which are induced by rhamnose. In some embodiments, the genetically engineered bacteria comprise multiple copies of different branched chain amino acid catabolism enzyme genes sequence(s) and/or transporter gene sequence(s) and/or BCAA binding protein gene sequence(s) and/or BCAA exporter gene sequence(s), e.g., as described herein, one or more of which are induced by rhamnose.

In a first example, the rhamnose inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the rhamnose inducible promoter drives expression under a first set of exogenous conditions, and the second promoter drives the expression under a second set of exogenous conditions. In second example, the rhamnose promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., rhamnose and IPTG). In another non-limiting example, the first inducing conditions are culture conditions, e.g., the presence of rhamnose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more rhamnose promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by rhamnose. In some embodiments, the gene sequence(s) encoding the branched chain amino acid catabolism enzyme(s) or branched chain amino acid transporter(s) and/or BCAA binding protein(s) and/or BCAA exporter(s) is present in the chromosome and operably linked to a promoter that is induced by rhamnose.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 106.

In some embodiments, the genetically engineered bacteria comprise one or more gene sequence(s) which are inducible through an Isopropyl β-D-1-thiogalactopyranoside (IPTG) inducible system or other compound which induced transcription from the Lac Promoter. IPTG is a molecular mimic of allolactose, a lactose metabolite that activates transcription of the lac operon. In contrast to allolactose, the sulfur atom in IPTG creates a non-hydrolyzable chemical blond, which prevents the degradation of IPTG, allowing the concentration to remain constant. IPTG binds to the lac repressor and releases the tetrameric repressor (lad) from the lac operator in an allosteric manner, thereby allowing the transcription of genes in the lac operon. Since IPTG is not metabolized by E. coli, its concentration stays constant and the rate of expression of Lac promoter-controlled is tightly controlled, both in vivo and in vitro. IPTG intake is independent on the action of lactose permease, since other transport pathways are also involved. Inducible expression from the PLac can be controlled or fine-tuned through the optimization of the ribosome binding site (RBS), as described herein. Other compounds which inactivate LacI, can be used instead of IPTG in a similar manner

In one embodiment, expression of one or more branched chain amino acid catabolism enzyme(s), e.g., kivD, leuDH, ilvE, L-AAD, BCKD, adh2, PadA, and/or YqhD, e.g., as described herein, is driven directly or indirectly by one or more IPTG inducible promoter(s). In one embodiment, the genetically engineered bacteria encode one or more branched chain amino acid transporter(s), e.g., livKHMGF and/or brnQ, described herein, whose expression is driven directly or indirectly by one or more IPTG inducible promoter(s). In one embodiment, the genetically engineered bacteria encode one or more branched chain amino acid binding protein(s), e.g., ilvJ, described herein, whose expression is driven directly or indirectly by one or more IPTG inducible promoter(s). In one embodiment, the genetically engineered bacteria encode one or more branched chain amino acid exporter(s) described herein, whose expression is driven directly or indirectly by one or more IPTG inducible promoter(s).

In some embodiments, the IPTG inducible promoter is useful for or induced during in vivo expression of the one or more protein(s) of interest. In some embodiments, expression of one or more branched chain amino acid catabolism enzyme(s) and/or branched chain amino acid transporter(s) is driven directly or indirectly by one or more IPTG inducible promoter(s) in vivo. In some embodiments, the promoter is directly or indirectly induced by a molecule (e.g., IPTG) that is co-administered with the genetically engineered bacteria of the invention.

In some embodiments, expression of one or more branched chain amino acid catabolism enzyme(s) and/or branched chain amino acid transporter(s) and/or BCAA binding protein(s) and/or BCAA exporter(s), is driven directly or indirectly by one or more IPTG inducible promoter(s) during in vitro growth, preparation, or manufacturing of the strain prior to in vivo administration. In some embodiments, the IPTG inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., IPTG, that is added to in the bacterial culture to induce expression and pre-load the bacterium with branched chain amino acid catabolism enzyme(s) prior to administration. In some embodiments, the cultures, which are induced by IPTG, are grown aerobically. In some embodiments, the cultures, which are induced by IPTG, are grown anaerobically.

In some embodiments, bacterial cell comprises two or more distinct branched chain amino acid catabolism cassette(s) or other polypeptide(s) of interest, one or more of which are induced by IPTG. In some embodiments, the genetically engineered bacteria comprise multiple copies of the same branched chain amino acid catabolism enzyme gene sequence(s) and/or transporter gene sequence(s), e.g., as described herein, which are induced IPTG. In some embodiments, the genetically engineered bacteria comprise multiple copies of different branched chain amino acid catabolism enzyme genes sequence(s) and/or transporter gene sequence(s) and/or other gene sequence(s) of interest, as described herein, one or more of which are induced by IPTG.

In a first example, the IPTG inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the IPTG inducible promoter drives expression under a first set of exogenous conditions, and the second promoter drives the expression under a second set of exogenous conditions. In second example, the IPTG promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., IPTG and IPTG). In another non-limiting example, the first inducing conditions are culture conditions, e.g., the presence of IPTG, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more IPTG promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by IPTG. In some embodiments, the gene sequence(s) encoding the branched chain amino acid catabolism enzyme(s) or branched chain amino acid transporter(s) is present in the chromosome and operably linked to a promoter that is induced by IPTG.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 107. In some embodiments, the IPTG inducible construct further comprises a gene encoding lad, which is divergently transcribed from the same promoter as the one or more one or more branched chain amino acid catabolism enzyme(s) and/or transporters described herein. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 109. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 109.

In some embodiments, the genetically engineered bacteria comprise one or more gene sequence(s) which are inducible through a tetracycline inducible system. The initial system Gossen and Bujard (Tight control of gene expression in mammalian cells by tetracycline-responsive promoters. Gossen M & Bujard H. PNAS, 1992 Jun. 15; 89(12):5547-51) developed is known as tetracycline off: in the presence of tetracycline, expression from a tet-inducible promoter is reduced. Tetracycline-controlled transactivator (tTA) was created by fusing tetR with the C-terminal domain of VP16 (virion protein 16) from herpes simplex virus. In the absence of tetracycline, the tetR portion of tTA will bind tetO sequences in the tet promoter, and the activation domain promotes expression. In the presence of tetracycline, tetracycline binds to tetR, precluding tTA from binding to the tetO sequences. Next, a reverse Tet repressor (rTetR), was developed which created a reliance on the presence of tetracycline for induction, rather than repression. The new transactivator rtTA (reverse tetracycline-controlled transactivator) was created by fusing rTetR with VP16. The tetracycline on system is also known as the rtTA-dependent system.

In one embodiment, expression of one or more branched chain amino acid catabolism enzyme(s), e.g., kivD, leuDH, ilvE, L-AAD, BCKD, adh2, PadA, and/or YqhD, e.g., as described herein, is driven directly or indirectly by one or more tetracycline inducible promoter(s). In one embodiment, the genetically engineered bacteria encode one or more branched chain amino acid transporter(s), e.g., livKHMGF and/or brnQ, described herein, whose expression is driven directly or indirectly by one or more tetracycline inducible promoter(s). In one embodiment, the genetically engineered bacteria encode one or more branched chain amino acid binding protein(s), e.g., ilvJ, described herein, whose expression is driven directly or indirectly by one or more tetracycline inducible promoter(s).

In one embodiment, the genetically engineered bacteria encode one or more branched chain amino acid exporter(s).

In some embodiments, the tetracycline inducible promoter is useful for or induced during in vivo expression of the one or more protein(s) of interest. In some embodiments, expression of one or more branched chain amino acid catabolism enzyme(s) and/or branched chain amino acid transporter(s) and or other polypeptide(s) of interest is driven directly or indirectly by one or more tetracycline inducible promoter(s) in vivo. In some embodiments, the promoter is directly or indirectly induced by a molecule (e.g., tetracycline) that is co-administered with the genetically engineered bacteria of the invention.

In some embodiments, expression of one or more branched chain amino acid catabolism enzyme(s) and/or branched chain amino acid transporter(s) and or other polypeptide(s) of interest, is driven directly or indirectly by one or more tetracycline inducible promoter(s) during in vitro growth, preparation, or manufacturing of the strain prior to in vivo administration. In some embodiments, the tetracycline inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., tetracycline, that is added to in the bacterial culture to induce expression and pre-load the bacterium with branched chain amino acid catabolism enzyme(s) prior to administration. In some embodiments, the cultures, which are induced by tetracycline, are grown aerobically. In some embodiments, the cultures, which are induced by tetracycline, are grown anaerobically.

In some embodiments, bacterial cell comprises two or more distinct branched chain amino acid catabolism cassette(s) and/or other polypeptide(s) of interest, one or more of which are induced by tetracycline. In some embodiments, the genetically engineered bacteria comprise multiple copies of the same branched chain amino acid catabolism enzyme gene sequence(s) and/or transporter gene sequence(s) and/or gene sequence(s) for the expression of other polypeptide(s) of interest, e.g., as described herein, which are induced by tetracycline. In some embodiments, the genetically engineered bacteria comprise multiple copies of different branched chain amino acid catabolism enzyme genes sequence(s) and/or transporter gene sequence(s) and gene sequence(s) for the expression of other polypeptide(s) of interest, e.g., as described herein, one or more of which are induced by tetracycline.

In a first example, the tetracycline inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the tetracycline inducible promoter drives expression under a first set of exogenous conditions, and the second promoter drives the expression under a second set of exogenous conditions. In second example, the tetracycline promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., tetracycline and IPTG). In another non-limiting example, the first inducing conditions are culture conditions, e.g., the presence of tetracycline, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more tetracycline promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by tetracycline. In some embodiments, the gene sequence(s) encoding the branched chain amino acid catabolism enzyme(s) or branched chain amino acid transporter(s) is present in the chromosome and operably linked to a promoter that is induced by tetracycline.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the bolded sequences of SEQ ID NO: 111 (tet promoter is in bold). In some embodiments, the tetracycline inducible construct further comprises a gene encoding AraC, which is divergently transcribed from the same promoter as the one or more one or more branched chain amino acid catabolism enzyme(s) and/or transporters described herein. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 111 in italics (Tet repressor is in italics). In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 111 in italics (Tet repressor is in italics).

In some embodiments, the genetically engineered bacteria comprise one or more gene sequence(s) whose expression is controlled by a temperature sensitive mechanism Thermoregulators are advantageous because of strong transcriptional control without the use of external chemicals or specialized media (see, e.g., Nemani et al., Magnetic nanoparticle hyperthermia induced cytosine deaminase expression in microencapsulated E. coli for enzyme-prodrug therapy; J Biotechnol. 2015 Jun. 10; 203: 32-40, and references therein). Thermoregulated protein expression using the mutant cI857 repressor and the pL and/or pR phage λ promoters have been used to engineer recombinant bacterial strains. The gene of interest cloned downstream of the λ promoters can then be efficiently regulated by the mutant thermolabile cI857 repressor of bacteriophage λ. At temperatures below 37° C., cI857 binds to the oL or regions of the pR promoter and blocks transcription by RNA polymerase. At higher temperatures, the functional cI857 dimer is destabilized, binding to the oL or oR DNA sequences is abrogated, and mRNA transcription is initiated. Inducible expression from the thermoregulated promoter can be controlled or further fine-tuned through the optimization of the ribosome binding site (RBS), as described herein.

In one embodiment, expression of one or more protein(s) of interest is driven directly or indirectly by one or more thermoregulated promoter(s). In one embodiment, expression of one or more branched chain amino acid catabolism enzyme(s), e.g., kivD, leuDH, ilvE, L-AAD, BCKD, adh2, PadA, and/or YqhD, e.g., as described herein, is driven directly or indirectly by one or more thermoregulated inducible promoter(s). In one embodiment, the genetically engineered bacteria encode one or more branched chain amino acid transporter(s), e.g., livKHMGF and/or brnQ, described herein, whose expression is driven directly or indirectly by one or more thermoregulated inducible promoter(s). In one embodiment, the genetically engineered bacteria encode one or more branched chain amino acid binding protein(s), e.g., ilvJ, described herein, whose expression is driven directly or indirectly by one or more thermoregulated inducible promoter(s). In one embodiment, the genetically engineered bacteria encode one or more branched chain amino acid exporter(s) described herein, whose expression is driven directly or indirectly by one or more thermoregulated inducible promoter(s).

In some embodiments, the thermoregulated promoter is useful for or induced during in vivo expression of the one or more protein(s) of interest. In some embodiments, expression of one or more branched chain amino acid catabolism enzyme(s) and/or branched chain amino acid transporter(s) and/or other protein(s) of interest is driven directly or indirectly by one or more thermoregulated promoter(s) in vivo.

In some embodiments, expression of one or more protein(s) of interest is driven directly or indirectly by one or more thermoregulated promoter(s) during in vitro growth, preparation, or manufacturing of the strain prior to in vivo administration. In some embodiments, it may be advantageous to shut off production of the one or more branched chain amino acid catabolism enzyme(s) and/or branched chain amino acid transporter(s). This can be done in a thermoregulated system by growing the strain at lower temperatures, e.g., 30 C. Expression can then be induced by elevating the temperature to 37 C and/or 42 C. In some embodiments, the thermoregulated promoter(s) are induced in culture, e.g., grown in a flask, fermenter or other appropriate culture vessel, e.g., used during cell growth, cell expansion, fermentation, recovery, purification, formulation, and/or manufacture. In some embodiments, the cultures, which are induced by temperatures between 37 C and 42 C, are grown aerobically. In some embodiments, the cultures, which are induced by induced by temperatures between 37 C and 42 C, are grown anaerobically.

In some embodiments, bacterial cell comprises two or more distinct branched chain amino acid catabolism cassette(s) or branched chain amino acid transporter(s) and/or cassette(s) for the expression of other protein(s) of interest, one or more of which are induced by temperature. In some embodiments, the genetically engineered bacteria comprise multiple copies of the same branched chain amino acid catabolism enzyme gene sequence(s) and/or transporter gene sequence(s) and/or gene sequence(s) for the expression of other proteins of interest, e.g., as described herein, which are induced by temperature. In some embodiments, the genetically engineered bacteria comprise multiple copies of different branched chain amino acid catabolism enzyme genes sequence(s) and/or transporter gene sequence(s) or other gene sequence(s) of interest, e.g., as described herein, one or more of which are induced by temperature.

In a first example, the temperature inducible promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the temperature inducible promoter drives expression under a first set of exogenous conditions, and the second promoter drives the expression under a second set of exogenous conditions. In second example, the temperature promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., temperature regulation and IPTG). In another non-limiting example, the first inducing conditions are culture conditions, e.g., the permissive temperature, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more temperature regulated promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by temperature. In some embodiments, the gene sequence(s) encoding the branched chain amino acid catabolism enzyme(s) or branched chain amino acid transporter(s) is present in the chromosome and operably linked to a promoter that is induced by temperature.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 112. In some embodiments, the thermoregulated construct further comprises a gene encoding mutant cI857 repressor, which is divergently transcribed from the same promoter as the one or more one or more branched chain amino acid catabolism enzyme(s) and/or transporters described herein. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 113. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 113.

In some embodiments, the genetically engineered bacteria comprise one or more gene sequence(s) which are indirectly inducible through a system driven by the PssB promoter. The Pssb promoter is active under aerobic conditions, and shuts off under anaerobic conditions.

This promoter can be used to express a gene of interest under aerobic conditions. This promoter can also be used to tightly control the expression of a gene product such that it is only expressed under anaerobic conditions. In this case, the oxygen induced PssB promoter induces the expression of a repressor, which represses the expression of a gene of interest. As a result, the gene of interest is only expressed in the absence of the repressor, i.e., under anaerobic conditions. This strategy has the advantage of an additional level of control for improved fine-tuning and tighter control. FIG. 80A depicts a schematic of the gene organization of a PssB promoter.

In one embodiment, expression of one or more branched chain amino acid catabolism enzyme(s), e.g., kivD, leuDH, ilvE, L-AAD, BCKD, adh2, PadA, and/or YqhD, e.g., as described herein, is driven directly or indirectly by one or more PssB promoter(s). In one embodiment, the genetically engineered bacteria encode one or more branched chain amino acid transporter(s), e.g., livKHMGF and/or brnQ, described herein, whose expression is driven directly or indirectly by one or more PssB promoter(s). In one embodiment, the genetically engineered bacteria encode one or more branched chain amino acid binding protein(s), e.g., ilvJ, described herein, whose expression is driven directly or indirectly by one or more PssB promoter(s). In one embodiment, the genetically engineered bacteria encode one or more branched chain amino acid exporter(s), described herein, whose expression is driven directly or indirectly by one or more PssB promoter(s).

In some embodiments, the PssB promoter is useful for or induced during in vivo expression of the one or more protein(s) of interest. In some embodiments, expression of one or more branched chain amino acid catabolism enzyme(s) and/or branched chain amino acid transporter(s) and/or other protein(s) of interest is driven directly or indirectly by one or more PssB promoter(s) in vivo. In some embodiments, the promoter is directly or indirectly induced by a molecule (e.g., arabinose) that is co-administered with the genetically engineered bacteria of the invention.

In some embodiments, expression of one or more branched chain amino acid catabolism enzyme(s) and/or branched chain amino acid transporter(s) and/or other protein(s) of interest, is driven directly or indirectly by one or more PssB promoter(s) during in vitro growth, preparation, or manufacturing of the strain prior to in vivo administration. In some embodiments, the PssB promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule, e.g., arabinose, that is added to in the bacterial culture to induce expression and pre-load the bacterium with branched chain amino acid catabolism enzyme(s) prior to administration. In some embodiments, the cultures, which are induced by arabinose, are grown aerobically. In some embodiments, the cultures, which are induced by arabinose, are grown anaerobically.

In some embodiments, bacterial cell comprises two or more distinct branched chain amino acid catabolism cassette(s) or other polypeptide(s) of interest, one or more of which are induced by arabinose. In some embodiments, the genetically engineered bacteria comprise multiple copies of the same branched chain amino acid catabolism enzyme gene sequence(s) and/or transporter gene sequence(s) and/or other gene sequence(s) of interest, e.g., as described herein, which are induced by one or more nutritional and/or chemical inducer(s) and/or metabolite(s). In some embodiments, the genetically engineered bacteria comprise multiple copies of different branched chain amino acid catabolism enzyme genes sequence(s) and/or transporter gene sequence(s), e.g., as described herein, one or more of which are induced by one or more nutritional and/or chemical inducer(s) and/or metabolite(s).

In a first example, the PssB promoter drives the expression of a construct comprising one or more polypeptides of interest described herein jointly with a second promoter, e.g., a second constitutive or inducible promoter. In some embodiments, two promoters are positioned proximally to the construct and drive its expression, wherein the PssB promoter drives expression under a first set of exogenous conditions, and the second promoter drives the expression under a second set of exogenous conditions. In second example, the PssB promoter drives the expression of one or more gene cassette(s) under a first inducing condition and another inducible promoter drives the expression of one or more of the same or different gene cassette(s) expressing one or more polypeptides of interest, under a second inducing condition. In both examples, the first and second conditions can be two sequential inducing culture conditions (i.e., during preparation of the culture in a flask, fermenter or other appropriate culture vessel, e.g., PssB and IPTG). In another non-limiting example, the first inducing conditions are culture conditions, e.g., the presence of arabinose, and the second inducing conditions are in vivo conditions. Such in vivo conditions include low-oxygen, microaerobic, or anaerobic conditions, presence of gut metabolites, and/or nutritional and/or chemical inducers and/or metabolites administered in combination with the bacterial strain. In some embodiments, the one or more PssB promoters drive expression of one or more protein(s) of interest, in combination with the FNR promoter driving the expression of the same gene sequence(s).

In some embodiments, the gene sequence(s) encoding the branched chain amino acid catabolism enzyme(s) or other polypeptide(s) of interest, are present on a plasmid and operably linked to a promoter that is induced by arabinose. In some embodiments, the gene sequence(s) encoding the branched chain amino acid catabolism enzyme(s) or branched chain amino acid transporter(s) is present in the chromosome and operably linked to a promoter that is induced by arabinose.

In another non-limiting example, this strategy can be used to control expression of thyA and/or dapA, e.g., to make a conditional auxotroph. The chromosomal copy of dapA or ThyA is knocked out. Under anaerobic conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can, for example be employed to allow survival of bacteria under anaerobic conditions, e.g., the gut, but prevent survival under aerobic conditions (biosafety switch). In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 116.

### Induction of Payloads During Strain Culture

In some embodiments, it is desirable to pre-induce activity of one or more branched chain amino acid catabolism enzyme(s), e.g., kivD, leuDH, ilvE, L-AAD, BCKD, adh2, PadA, and/or YqhD, and/or branched chain amino acid transporter(s) and/or other protein(s) of interest prior to administration. Such branched chain amino acid catabolism enzyme gene(s) and/or branched chain amino acid transporter(s) and/or other protein(s) of interest can be an effector intended for secretion or can be an enzyme which catalyzes a metabolic reaction to produce an effector. In other embodiments, the protein of interest is an enzyme which catabolizes a harmful metabolite. In such situations, the strains are pre-loaded with active payload or protein of interest. In such instances, the genetically engineered bacteria of the invention express one or more branched chain amino acid catabolism enzyme(s) and/or branched chain amino acid transporter(s) and/or other protein(s) of interest, under conditions provided in bacterial culture during cell growth, expansion, purification, fermentation, and/or manufacture prior to administration in vivo. Such culture conditions can be provided in a flask, fermenter or other appropriate culture vessel, e.g., used during cell growth, cell expansion, fermentation, recovery, purification, formulation, and/or manufacture. As used herein, the term “bacterial culture” or bacterial cell culture” or “culture” refers to bacterial cells or microorganisms, which are maintained or grown in vitro during several production processes, including cell growth, cell expansion, recovery, purification, fermentation, and/or manufacture. As used herein, the term “fermentation” refers to the growth, expansion, and maintenance of bacteria under defined conditions. Fermentation may occur under a number of different cell culture conditions, including anaerobic or low oxygen or oxygenated conditions, in the presence of inducers, nutrients, at defined temperatures, and the like.

Culture conditions are selected to achieve optimal activity and viability of the cells, while maintaining a high cell density (high biomass) yield. A number of different cell culture conditions and operating parameters are monitored and adjusted to achieve optimal activity, high yield and high viability, including oxygen levels (e.g., low oxygen, microaerobic, aerobic), temperature of the medium, and nutrients and/or different growth media, chemical and/or nutritional inducers and other components provided in the medium.

In some embodiments, the one or more branched chain amino acid catabolism enzyme(s) and/or other protein(s) of interest and are directly or indirectly induced, while the strains are grown up for in vivo administration. Without wishing to be bound by theory, pre-induction may boost in vivo activity. In contrast, if a strain is pre-induced and preloaded, the strains are already fully active, allowing for greater activity more quickly as the bacteria reach the region of the intestine in which they are active, e.g., the gut. Ergo, no transit time is “wasted”, in which the strain is not optimally active. As the bacteria continue to move through the intestine, in vivo induction occurs under environmental conditions of the gut (e.g., low oxygen, or in the presence of gut metabolites).

In one embodiment, expression of one or more branched chain amino acid catabolism enzyme(s) and/or branched chain amino acid transporter(s) and/or other protein(s) of interest, is induced during cell growth, cell expansion, fermentation, recovery, purification, formulation, and/or manufacture. In one embodiment, induction of one or more promoters, each driving expression of one or more proteins of interest, occurs during cell growth, cell expansion, fermentation, recovery, purification, formulation, and/or manufacture. In one embodiment, expression of one or more branched chain amino acid catabolism enzyme(s) and/or branched chain amino acid transporter(s) and/or other protein(s) of interest is driven from the same promoter. In one embodiment, expression of one or more branched chain amino acid catabolism enzyme(s) and/or branched chain amino acid transporter(s) and/or other protein(s) of interest is driven from two or more copies of the same promoter. In one embodiment, expression of two or more branched chain amino acid catabolism enzyme(s) and/or branched chain amino acid transporter(s) and/or other protein(s) of interest is driven from two or more copies of the same promoter and the two or more payloads are the same. In one embodiment, expression of two or more branched chain amino acid catabolism enzyme(s) and/or branched chain amino acid transporter(s) and/or other protein(s) of interest is driven from the two or more copies of the same promoter and the two or more branched chain amino acid catabolism enzyme(s) and/or branched chain amino acid transporter(s) and/or other protein(s) of interest are different. In one embodiment, expression of two or more branched chain amino acid catabolism enzyme(s) and/or branched chain amino acid transporter(s) and/or other protein(s) of interest is driven from two or more copies of different promoter(s). In one embodiment, expression of one or more branched chain amino acid catabolism enzyme(s) and/or branched chain amino acid transporter(s) and/or other protein(s) of interest is driven from two or more different promoter(s) and the two or more branched chain amino acid catabolism enzyme(s) and/or branched chain amino acid transporter(s) and/or other protein(s) of interest are the same. In one embodiment, expression of two or more branched chain amino acid catabolism enzyme(s) and/or branched chain amino acid transporter(s) and/or other protein(s) of interest is driven from two or more different promoter(s) and the two or more branched chain amino acid catabolism enzyme(s) and/or branched chain amino acid transporter(s) and/or other protein(s) of interest are different. In one embodiment, expression of two or more of the same or different branched chain amino acid catabolism enzyme(s) and/or branched chain amino acid transporter(s) and/or other protein(s) of interest is driven from the two or more copies of the same or different promoters. Payloads are expressed either from plasmid(s), the bacterial chromosome, or both.

In some embodiments, the strains are administered without any pre-induction protocols during strain growth prior to in vivo administration.

### Anaerobic Induction

In some embodiments, cells are induced under strictly anaerobic or low oxygen conditions in culture. In such instances, cells are grown (e.g., for 1.5 to 3 hours) until they have reached a certain OD, e.g., ODs within the range of 0.1 to 10, indicating a certain density e.g., ranging from 1×10̂8 to 1×10̂11, and exponential growth and are then switched to strictly anaerobic or low oxygen conditions for approximately 3 to 5 hours. In some embodiments, strains are induced under strictly anaerobic or low oxygen conditions, e.g. to induce FNR promoter activity and drive expression of one or more branched chain amino acid catabolism enzyme(s) and/or branched chain amino acid transporter(s) and/or BCAA transporters under the control of one or more FNR promoters.

In one embodiment, expression of one or more one or more branched chain amino acid catabolism enzyme(s) e.g., kivD, leuDH, ilvE, L-AAD, BCKD, adh2, PadA, and/or YqhD, and/or branched chain amino acid transporter(s), e.g., livKHMGF and/or brnQ, and/or other protein(s) of interest is under the control of one or more FNR promoter(s) and is induced during cell growth, cell expansion, fermentation, recovery, purification, formulation, and/or manufacture under strictly anaerobic or low oxygen conditions. In one embodiment, expression of several different branched chain amino acid catabolism enzyme(s) and/or branched chain amino acid transporter(s) and/or other protein(s) of interest is under the control of one or more FNR promoter(s) and is induced during cell growth, cell expansion, fermentation, recovery, purification, formulation, and/or manufacture under strictly anaerobic or low oxygen conditions.

Without wishing to be bound by theory, strains that comprise one or more branched chain amino acid catabolism enzyme gene(s) and/or branched chain amino acid transporter(s) and/or other protein(s) of interest under the control of an FNR promoter, may allow expression of branched chain amino acid catabolism enzyme(s) and/or branched chain amino acid transporter(s) and/or other protein(s) of interest from these promoters in vitro, under strictly anaerobic or low oxygen culture conditions, and in vivo, under the low oxygen conditions found in the gut.

In some embodiments, promoters inducible by arabinose, IPTG, rhamnose, tetracycline, and/or other chemical and/or nutritional inducers can be induced under strictly anaerobic or low oxygen conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more gene of interest sequence(s) under the control of one or more FNR promoter(s) and one or more same or different gene of interest sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) and/or under the control of one or more FNR promoter(s), and one or more same or different payload gene sequence(s) under the control of a one or more constitutive promoter(s) described herein. In some embodiments, strains may comprise one or more payload gene sequence(s) under the control of an FNR promoter and one or more same or different payload gene sequence(s) under the control of a one or more thermoregulated promoter(s) described herein.

In one embodiment, expression of one or more one or more branched chain amino acid catabolism enzyme(s) and/or branched chain amino acid transporter(s) and/or other protein(s) of interest is under the control of one or more promoter(s) regulated by chemical and/or nutritional inducers and is induced during cell growth, cell expansion, fermentation, recovery, purification, formulation, and/or manufacture under strictly anaerobic and/or low oxygen conditions. In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In one embodiment, induction of two or more copies of the same promoters or two or more different promoters, each driving expression of the same or different proteins of interest, occurs during cell growth, cell expansion, fermentation, recovery, purification, formulation, and/or manufacture, e.g., under strictly anaerobic and/or low oxygen conditions. In one embodiment, expression of two or more branched chain amino acid catabolism enzyme(s) and/or branched chain amino acid transporter(s) and/or other protein(s) of interest is driven from two or more copies of the same promoter, e.g., under strictly anaerobic and/or low oxygen conditions. In one embodiment, expression of two or more branched chain amino acid catabolism enzyme(s) and/or branched chain amino acid transporter(s) and/or other protein(s) of interest under strictly anaerobic and/or low oxygen conditions is driven from two or more copies of the same promoter and the payloads are the same. In one embodiment, expression of two or more branched chain amino acid catabolism enzyme(s) and/or branched chain amino acid transporter(s) and/or other protein(s) of interest under strictly anaerobic and/or low oxygen conditions is driven from two or more copies of the same promoter and the payloads are different. In one embodiment, expression of two or more branched chain amino acid catabolism enzyme(s) and/or branched chain amino acid transporter(s) and/or other protein(s) of interest under strictly anaerobic and/or low oxygen conditions is driven from two or more different promoter(s). In one embodiment, expression of two or more branched chain amino acid catabolism enzyme(s) and/or branched chain amino acid transporter(s) and/or other protein(s) of interest under strictly anaerobic and/or low oxygen conditions is driven from two or more different promoter(s) and the branched chain amino acid catabolism enzyme(s) and/or branched chain amino acid transporter(s) and/or other protein(s) of interest are the same. In one embodiment, expression of one or more branched chain amino acid catabolism enzyme(s) and/or branched chain amino acid transporter(s) and/or other protein(s) of interest under strictly anaerobic and/or low oxygen conditions is driven from two or more different promoter(s), and the branched chain amino acid catabolism enzyme(s) and/or branched chain amino acid transporter(s) and/or other protein(s) of interest are different. In one embodiment, expression of one or more of the same or different branched chain amino acid catabolism enzyme(s) and/or branched chain amino acid transporter(s) and/or other protein(s) of interest, under strictly anaerobic and/or low oxygen conditions, is driven from the one or more same or different promoters. Payloads are expressed either from plasmid(s), the bacterial chromosome, or both.

In one embodiment, strains may comprise a combination of gene sequence(s), some of which are under control of a first inducible promoter and others which are under control of a second inducible promoter, both induced by chemical and/or nutritional inducers, under strictly anaerobic or low oxygen conditions. In some embodiments, the strains comprise gene sequence(s) under the control of a. third inducible promoter, e.g., a strictly anaerobic/low oxygen promoter, e.g., FNR promoter. In one embodiment, strains may comprise a combination of gene sequence(s), some of which are under control of a first inducible promoter, e.g., a chemically induced promoter or a low oxygen promoter and others which are under control of a second inducible promoter, e.g. a temperature sensitive promoter. In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains comprise payload under the control of one or more constitutive promoter(s) active under low oxygen conditions. In some embodiments, the bacteria strains comprise one or more payload under the control of one or more constitutive promoter(s) active and one or more inducible promoter(s), e.g., FNR and/or chemically, nutritionally and/or metabolite inducible and/or thermo regulated, under low oxygen conditions.

### Aerobic Induction

In some embodiments, it is desirable to prepare, pre-load and pre-induce the strains under aerobic conditions. This allows more efficient growth and viability, and, in some cases, reduces the build-up of toxic metabolites. In such instances, cells are grown (e.g., for 1.5 to 3 hours) until they have reached a certain OD, e.g., ODs within the range of 0.1 to 10, indicating a certain density e.g., ranging from 1×10̂8 to 1×10̂11, and exponential growth and are then induced through the addition of the inducer or through other means, such as shift to a permissive temperature, for approximately 3 to 5 hours.

In some embodiments, promoters inducible by one or more chemical and/or nutritional inducer(s) and or metabolite(s), e.g., by arabinose, IPTG, rhamnose, tetracycline, and/or other chemical and/or nutritional inducers described herein or known in the art can be induced under aerobic conditions in the presence of the chemical and/or nutritional and/or metabolite inducer during cell growth, cell expansion, fermentation, recovery, purification, formulation, and/or manufacture. In one embodiment, expression of one or more branched chain amino acid catabolism enzyme(s) and/or branched chain amino acid transporter(s) and/or other protein(s) of interest is under the control of one or more promoter(s) regulated by chemical and/or nutritional inducers and is induced during cell growth, cell expansion, fermentation, recovery, purification, formulation, and/or manufacture under aerobic conditions.

In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In some embodiments, promoters regulated by temperature are induced during cell growth, cell expansion, fermentation, recovery, purification, formulation, and/or manufacture. In one embodiment, expression of one or more branched chain amino acid catabolism enzyme(s) and/or branched chain amino acid transporter(s) and/or other protein(s) of interest is driven directly or indirectly by one or more thermoregulated promoter(s) and is induced during cell growth, cell expansion, fermentation, recovery, purification, formulation, and/or manufacture under aerobic conditions.

In one embodiment, induction of two or more copies of the same promoters or two or more different promoters, each driving expression of the same or different proteins of interest, occurs during cell growth, cell expansion, fermentation, recovery, purification, formulation, and/or manufacture, e.g., under aerobic conditions. In one embodiment, expression of two or more branched chain amino acid catabolism enzyme(s), e.g., kivD, leuDH, ilvE, L-AAD, BCKD, adh2, PadA, and/or YqhD, and/or one or more branched chain amino acid transporter(s), e.g., livKHMGF and/or brnQ, is driven from two or more copies of the same promoter, e.g., under aerobic conditions. In one embodiment, expression of two or more branched chain amino acid catabolism enzyme(s) and/or branched chain amino acid transporter(s) and/or other protein(s) of interest under aerobic conditions is driven from two or more copies of the same promoter and the payloads are the same. In one embodiment, expression of two or more branched chain amino acid catabolism enzyme(s) and/or branched chain amino acid transporter(s) and/or other protein(s) of interest under aerobic conditions is driven from two or more copies of the same promoter and the payloads are different. In one embodiment, expression of two or more branched chain amino acid catabolism enzyme(s) and/or branched chain amino acid transporter(s) and/or other protein(s) of interest under aerobic conditions is driven from two or more different promoter(s). In one embodiment, expression of two or more branched chain amino acid catabolism enzyme(s) and/or branched chain amino acid transporter(s) and/or other protein(s) of interest under aerobic conditions is driven from two or more different promoter(s) and the branched chain amino acid catabolism enzyme(s) and/or branched chain amino acid transporter(s) and/or other protein(s) of interest are the same. In one embodiment, expression of one or more branched chain amino acid catabolism enzyme(s) and/or branched chain amino acid transporter(s) and/or other protein(s) of interest under aerobic conditions is driven from two or more different promoter(s), and the branched chain amino acid catabolism enzyme(s) and/or branched chain amino acid transporter(s) and/or other protein(s) of interest are different. In one embodiment, expression of one or more of the same or different branched chain amino acid catabolism enzyme(s) and/or branched chain amino acid transporter(s) and/or other protein(s) of interest, under aerobic conditions, is driven from the one or more same or different promoters. Payloads are expressed either from plasmid(s), the bacterial chromosome, or both.

In one embodiment, strains may comprise a combination of gene sequence(s) encoding one or more one or more branched chain amino acid catabolism enzyme(s), e.g., kivD, leuDH, ilvE, L-AAD, BCKD, adh2, PadA, and/or YqhD, and/or branched chain amino acid transporter(s), e.g., livKHMGF and/or brnQ, some of which are under control of a first inducible promoter and others which are under control of a second inducible promoter, both induced under aerobic conditions. In some embodiments, a strain comprises three or more different promoters which are induced under aerobic culture conditions.

In one embodiment, strains may comprise a combination of gene sequence(s), some of which are under control of a first inducible promoter and others which are under control of a second inducible promoter, both induced by chemical and/or nutritional inducers. In one embodiment, strains may comprise a combination of gene sequence(s), some of which are under control of a first inducible promoter, e.g. a chemically inducible promoter, and others which are under control of a second inducible promoter, e.g. a temperature sensitive promoter under aerobic culture conditions. In some embodiments two or more chemically induced promoter gene sequence(s) are combined with a thermoregulated construct described herein. In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) and/or BCAA transporter gene sequence(s) and/or transcriptional regulator gene sequence(s) under the control of an FNR promoter and one or more payload gene sequence(s) and/or BCAA transporter gene sequence(s) and/or transcriptional regulator gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload and or BCAA transporter sequence(s) under the control of one or more constitutive promoter(s) active under aerobic conditions.

In some embodiments, genetically engineered strains comprise gene sequence(s) which are induced under aerobic culture conditions. In some embodiments, these strains further comprise FNR inducible gene sequence(s) for in vivo activation in the gut. In some embodiments, these strains do not further comprise FNR inducible gene sequence(s) for in vivo activation in the gut.

In some embodiments, genetically engineered strains comprise gene sequence(s), which are arabinose inducible under aerobic culture conditions. In some embodiments, these strains do not further comprise FNR inducible gene sequence(s) for in vivo activation in the gut.

In some embodiments, genetically engineered strains comprise gene sequence(s), which are IPTG inducible under aerobic culture conditions. In some embodiments, these strains further comprise FNR inducible gene sequence(s) for in vivo activation in the gut. In some embodiments, these strains do not further comprise FNR inducible gene sequence(s) for in vivo activation in the gut.

In some embodiments, genetically engineered strains comprise gene sequence(s) which are arabinose inducible under aerobic culture conditions. In some embodiments, such a strain further comprises sequence(s) which are IPTG inducible under aerobic culture conditions. In some embodiments, these strains further comprise FNR inducible gene payload and/or BCAA transporter sequence(s) for in vivo activation in the gut. In some embodiments, these strains do not further comprise FNR inducible gene sequence(s) for in vivo activation in the gut.

As evident from the above non-limiting examples, genetically engineered strains comprise inducible gene sequence(s) which can be induced numerous combinations. For example, rhamnose or tetracycline can be used as an inducer with the appropriate promoters in addition or in lieu of arabinose and/or IPTG or with thermoregulation. Additionally, such bacterial strains can also be induced with the chemical and/or nutritional inducers under anaerobic conditions.

### Microaerobic Induction

In some embodiments, viability, growth, and activity are optimized by pre-inducing the bacterial strain under microaerobic conditions. In some embodiments, microaerobic conditions are best suited to “strike a balance” between optimal growth, activity and viability conditions and optimal conditions for induction; in particular, if the expression of the one or more branched chain amino acid catabolism enzyme(s) and/or branched chain amino acid transporter(s) and/or other protein(s) of interest and/or BCAA transporter(s) are driven by an anaerobic and/or low oxygen promoter, e.g., a FNR promoter. In such instances, cells are grown (e.g., for 1.5 to 3 hours) until they have reached a certain OD, e.g., ODs within the range of 0.1 to 10, indicating a certain density e.g., ranging from 1×10̂8 to 1×10̂11, and exponential growth and are then induced through the addition of the inducer or through other means, such as shift to at a permissive temperature, for approximately 3 to 5 hours.

In one embodiment, expression of one or more branched chain amino acid catabolism enzyme(s) and/or branched chain amino acid transporter(s) and/or other protein(s) of interest is under the control of one or more FNR promoter(s) and is induced during cell growth, cell expansion, fermentation, recovery, purification, formulation, and/or manufacture under microaerobic conditions.

Without wishing to be bound by theory, strains that comprise one or more branched chain amino acid catabolism enzyme(s) and/or branched chain amino acid transporter(s) and/or other protein(s) of interest, e.g., one or more branched chain amino acid catabolism enzyme(s) and/or other polypeptides of interest, under the control of an FNR promoter, may allow expression of branched chain amino acid catabolism enzyme(s) and/or branched chain amino acid transporter(s) and/or other protein(s) of interest from these promoters in vitro, under microaerobic culture conditions, and in vivo, under the low oxygen conditions found in the gut.

In some embodiments, promoters inducible by arabinose, IPTG, rhamnose, tetracycline, and/or other chemical and/or nutritional inducers can be induced under microaerobic conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) sequence(s) under the control of one or more FNR promoter(s) and one or more payload gene sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) under the control of one or more FNR promoter(s), and one or more payload gene sequence(s) under the control of a one or more constitutive promoter(s) described herein. In some embodiments, strains may comprise one or more payload gene sequence(s) under the control of an FNR promoter and one or more payload gene sequence(s) under the control of a one or more thermoregulated promoter(s) described herein.

In one embodiment, expression of one or more branched chain amino acid catabolism enzyme(s) e.g., kivD, leuDH, ilvE, L-AAD, BCKD, adh2, PadA, and/or YqhD, and/or one or more branched chain amino acid transporter(s), e.g., livKHMGF and/or brnQ, is under the control of one or more promoter(s) regulated by chemical and/or nutritional inducers and is induced during cell growth, cell expansion, fermentation, recovery, purification, formulation, and/or manufacture under microaerobic conditions.

In one embodiment, induction of two or more copies of the same promoters or two or more different promoters, each driving expression of the same or different proteins of interest, occurs during cell growth, cell expansion, fermentation, recovery, purification, formulation, and/or manufacture, e.g., under microaerobic conditions. In one embodiment, expression of two or more branched chain amino acid catabolism enzyme(s) and/or branched chain amino acid transporter(s) and/or other protein(s) of interest is driven from two or more copies of the same promoter, e.g., under microaerobic conditions. In one embodiment, expression of two or more branched chain amino acid catabolism enzyme(s) and/or branched chain amino acid transporter(s) and/or other protein(s) of interest under microaerobic conditions is driven from two or more copies of the same promoter and the payloads are the same. In one embodiment, expression of two or more branched chain amino acid catabolism enzyme(s) and/or branched chain amino acid transporter(s) and/or other protein(s) of interest under microaerobic conditions is driven from two or more copies of the same promoter and the payloads are different. In one embodiment, expression of two or more branched chain amino acid catabolism enzyme(s) and/or branched chain amino acid transporter(s) and/or other protein(s) of interest under microaerobic conditions is driven from two or more different promoter(s). In one embodiment, expression of two or more branched chain amino acid catabolism enzyme(s) and/or branched chain amino acid transporter(s) and/or other protein(s) of interest under microaerobic conditions is driven from two or more different promoter(s) and the branched chain amino acid catabolism enzyme(s) and/or branched chain amino acid transporter(s) and/or other protein(s) of interest are the same. In one embodiment, expression of one or more branched chain amino acid catabolism enzyme(s) and/or branched chain amino acid transporter(s) and/or other protein(s) of interest under microaerobic conditions is driven from two or more different promoter(s), and the branched chain amino acid catabolism enzyme(s) and/or branched chain amino acid transporter(s) and/or other protein(s) of interest are different. In one embodiment, expression of one or more of the same or different branched chain amino acid catabolism enzyme(s) and/or branched chain amino acid transporter(s) and/or other protein(s) of interest, under microaerobic conditions, is driven from the one or more same or different promoters. Payloads are expressed either from plasmid(s), the bacterial chromosome, or both.

In one embodiment, strains may comprise a combination of gene sequence(s), some of which are under control of a first inducible promoter and others which are under control of a second inducible promoter, both induced by chemical and/or nutritional inducers, under microaerobic conditions. In one embodiment, strains may comprise a combination of gene sequence(s), some of which are under control of a first inducible promoter and others which are under control of a second inducible promoter, both induced by chemical and/or nutritional inducers. In some embodiments, the strains comprise gene sequence(s) under the control of a third inducible promoter, e.g., an anaerobic/low oxygen promoter or microaerobic promoter, e.g., FNR promoter. In one embodiment, strains may comprise a combination of gene sequence(s), some of which are under control of a first inducible promoter, e.g., a chemically induced promoter or a low oxygen or microaerobic promoter and others which are under control of a second inducible promoter, e.g. a temperature sensitive promoter. In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload under the control of one or more constitutive promoter(s) active under low oxygen conditions.

Induction of Strains Using Phasing, Pulsing and/or Cycling

In some embodiments, cycling, phasing, or pulsing techniques are employed during cell growth, expansion, recovery, purification, fermentation, and/or manufacture to efficiently induce and grow the strains prior to in vivo administration. This method is used to “strike a balance” between optimal growth, activity, cell health, and viability conditions and optimal conditions for induction; in particular, if growth, cell health or viability are negatively affected under inducing conditions. In such instances, cells are grown (e.g., for 1.5 to 3 hours) in a first phase or cycle until they have reached a certain OD, e.g., ODs within the range of 0.1 to 10, indicating a certain density e.g., ranging from 1×10̂8 to 1×10̂11, and are then induced through the addition of the inducer or through other means, such as shift to a permissive temperature (if a promoter is thermoregulated), or change in oxygen levels (e.g., reduction of oxygen level in the case of induction of an FNR promoter driven construct) for approximately 3 to 5 hours. In a second phase or cycle, conditions are brought back to the original conditions which support optimal growth, cell health and viability. Alternatively, if a chemical and/or nutritional inducer is used, then the culture can be spiked with a second dose of the inducer in the second phase or cycle.

In some embodiments, two cycles of optimal conditions and inducing conditions are employed (i.e., growth, induction, recovery and growth, induction). In some embodiments, three cycles of optimal conditions and inducing conditions are employed. In some embodiments, four or more cycles of optimal conditions and inducing conditions are employed. In a non-liming example, such cycling and/or phasing is used for induction under anaerobic and/or low oxygen conditions (e.g., induction of FNR promoters). In one embodiment, cells are grown to the optimal density and then induced under anaerobic and/or low oxygen conditions. Before growth and/or viability are negatively impacted due to stressful induction conditions, cells are returned to oxygenated conditions to recover, after which they are then returned to inducing anaerobic and/or low oxygen conditions for a second time. In some embodiments, these cycles are repeated as needed.

In some embodiments, growing cultures are spiked once with the chemical and/or nutritional inducer. In some embodiments, growing cultures are spiked twice with the chemical and/or nutritional inducer. In some embodiments, growing cultures are spiked three or more times with the chemical and/or nutritional inducer. In a non-limiting example, cells are first grown under optimal growth conditions up to a certain density, e.g., for 1.5 to 3 hour) to reached an of 0.1 to 10, until the cells are at a density ranging from 1×10̂8 to 1×10̂11. Then the chemical inducer, e.g., arabinose or IPTG, is added to the culture. After 3 to 5 hours, an additional dose of the inducer is added to re-initiate the induction. Spiking can be repeated as needed.

In some embodiments, phasing or cycling changes in temperature in the culture. In another embodiment, adjustment of temperature may be used to improve the activity of a payload. For example, lowering the temperature during culture may improve the proper folding of the payload. In such instances, cells are first grown at a temperature optimal for growth (e.g., 37 C). In some embodiments, the cells are then induced, e.g., by a chemical inducer, to express the payload. Concurrently or after a set amount of induction time, the temperature in the media is lowered, e.g., between 25 and 35 C, to allow improved folding of the expressed payload.

In some embodiments, one or more branched chain amino acid catabolism enzymes e.g., kivD, leuDH, ilvE, L-AAD, BCKD, adh2, PadA, and/or YqhD, and/or one or more branched chain amino acid transporter(s) are under the control of different inducible promoters, for example two different chemical inducers. In other embodiments, the branched chain amino acid catabolism enzyme and/or transporter is induced under low oxygen conditions or microaerobic conditions and a second payload is induced by a chemical inducer.

In one embodiment, expression of one or more branched chain amino acid catabolism enzyme(s) and/or branched chain amino acid transporter(s) and/or other protein(s) of interest is under the control of one or more FNR promoter(s) and is induced during cell growth, cell expansion, fermentation, recovery, purification, formulation, and/or manufacture by using phasing or cycling or pulsing or spiking techniques.

In some embodiments, promoters inducible by arabinose, IPTG, rhamnose, tetracycline, and/or other chemical and/or nutritional inducers can be induced through the employment of phasing or cycling or pulsing or spiking techniques in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) under the control of one or more FNR promoter(s) and one or more payload gene sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) under the control of one or more FNR promoter(s), and one or more payload gene sequence(s) and/or BCAA transporter gene sequence(s) and/or transcriptional regulator gene sequence(s) under the control of a one or more constitutive promoter(s) described herein and are induced through the employment of phasing or cycling or pulsing or spiking techniques. In some embodiments, strains may comprise one or more payload gene sequence(s) under the control of an FNR promoter and one or more payload gene sequence(s) under the control of a one or more thermoregulated promoter(s) described herein, and are induced through the employment of phasing or cycling or pulsing or spiking techniques.

Any of the strains described herein can be grown through the employment of phasing or cycling or pulsing or spiking techniques. In one embodiment, expression of one or more branched chain amino acid catabolism enzyme(s) and/or branched chain amino acid transporter(s) and/or other protein(s) of interest is under the control of one or more promoter(s) regulated by chemical and/or nutritional inducers and is induced during cell growth, cell expansion, fermentation, recovery, purification, formulation, and/or manufacture under anaerobic and/or low oxygen conditions.

In one embodiment, induction of two or more copies of the same promoters or two or more different promoters, each driving expression of the same or different proteins of interest, occurs during cell growth, cell expansion, fermentation, recovery, purification, formulation, and/or manufacture, e.g, through the employment of phasing or cycling or pulsing or spiking techniques. In one embodiment, expression of two or more branched chain amino acid catabolism enzyme(s) and/or branched chain amino acid transporter(s) and/or other protein(s) of interest is driven from two or more copies of the same promoter, through the employment of phasing or cycling or pulsing or spiking techniques. In one embodiment, expression of two or more branched chain amino acid catabolism enzyme(s) and/or branched chain amino acid transporter(s) and/or other protein(s) of interest, regulated through the employment of phasing or cycling or pulsing or spiking techniques, is driven from two or more copies of the same promoter and the payloads are the same. In one embodiment, expression of two or more branched chain amino acid catabolism enzyme(s) and/or branched chain amino acid transporter(s) and/or other protein(s) of interest, regulated through the employment of phasing or cycling or pulsing or spiking techniques is driven from two or more copies of the same promoter and the payloads are different. In one embodiment, expression of two or more branched chain amino acid catabolism enzyme(s) and/or branched chain amino acid transporter(s) and/or other protein(s) of interest, regulated through the employment of phasing or cycling or pulsing or spiking techniques is driven from two or more different promoter(s). In one embodiment, expression of two or more branched chain amino acid catabolism enzyme(s) and/or branched chain amino acid transporter(s) and/or other protein(s) of interest, regulated through the employment of phasing or cycling or pulsing or spiking techniques, is driven from two or more different promoter(s) and the branched chain amino acid catabolism enzyme(s) and/or branched chain amino acid transporter(s) and/or other protein(s) of interest are the same. In one embodiment, expression of one or more branched chain amino acid catabolism enzyme(s) and/or branched chain amino acid transporter(s) and/or other protein(s) of interest, regulated through the employment of phasing or cycling or pulsing or spiking techniques, is driven from two or more different promoter(s), and the branched chain amino acid catabolism enzyme(s) and/or branched chain amino acid transporter(s) and/or other protein(s) of interest are different. In one embodiment, expression of one or more of the same or different branched chain amino acid catabolism enzyme(s) and/or branched chain amino acid transporter(s) and/or other protein(s) of interest, regulated through the employment of phasing or cycling or pulsing or spiking techniques, is driven from the one or more same or different promoters. Payloads are expressed either from plasmid(s), the bacterial chromosome, or both.

In one embodiment, strains may comprise a combination of gene sequence(s), some of which are under control of a first inducible promoter and others which are under control of a second inducible promoter, both induced by chemical and/or nutritional inducers, through the employment of phasing or cycling or pulsing or spiking techniques. In one embodiment, strains may comprise a combination of gene sequence(s), some of which are under control of a first inducible promoter and others which are under control of a second inducible promoter, both induced by chemical and/or nutritional inducers through the employment of phasing or cycling or pulsing or spiking techniques. In some embodiments, the strains comprise gene sequence(s) under the control of a third inducible promoter, e.g., an anaerobic/low oxygen promoter, e.g., FNR promoter. In one embodiment, strains may comprise a combination of gene sequence(s), some of which are under control of a first inducible promoter, e.g., a chemically induced promoter or a low oxygen promoter and others which are under control of a second inducible promoter, e.g. a temperature sensitive promoter. In one embodiment, strains may comprise a combination of gene sequence(s), some of which are under control of a first inducible promoter, e.g., a FNR promoter and others which are under control of a second inducible promoter, e.g. a temperature sensitive promoter. In one embodiment, strains may comprise a combination of gene sequence(s), some of which are under control of a first inducible promoter, e.g., a chemically induced and others which are under control of a second inducible promoter, e.g. a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload sequence(s) under the control of one or more constitutive promoter(s) active under low oxygen conditions. Any of the strains described in these embodiments may be induced through the employment of phasing or cycling or pulsing or spiking techniques.

### Aerobic Induction of the FNR Promoter

FNRS24Y is a mutated form of FNR which is more resistant to inactivation by oxygen, and therefore can activate FNR promoters under aerobic conditions (see e.g., Jervis A J The O2 sensitivity of the transcription factor FNR is controlled by Ser24 modulating the kinetics of [4Fe-4S] to [2Fe-2S] conversion, Proc Natl Acad Sci USA. 2009 Mar. 24; 106(12):4659-64, the contents of which is herein incorporated by reference in its entirety). In some embodiments, oxygen bypass system shown and described in FIG. 79 is used. In this oxygen bypass system, FNRS24Y is induced by addition of arabinose and then drives the expression a branched chain amino acid catabolizing enzyme (POI1) and/or a transporter and/or exporter (POI2) by binding and activating the FNR promoter under aerobic conditions. Thus, strains can be grown, produced or manufactured efficiently under aerobic conditions, while being effectively pre-induced and pre-loaded, as the system takes advantage of the strong FNR promoter resulting in of high levels of expression of POI1 and PO2. This system does not interfere with or compromise in vivo activation, since the mutated FNRS24Y is no longer expressed in the absence of arabinose, and wild type FNR then binds to the FNR promoter and drives expression of POI1 and POI2.

In some embodiments, FNRS24Y is expressed during aerobic culture growth and induces a gene of interest. In other embodiments described herein, a second payload expression can also be induced aerobically, e.g., by arabinose. In a non-limiting example, a protein of interest and FNRS24Y can in some embodiments be induced simultaneously, e.g., from an arabinose inducible promoter. In some embodiments, FNRS24Y and the protein of interest are transcribed as a bicistronic message whose expression is driven by an arabinose promoter. In some embodiments, FNRS24Y is knocked into the arabinose operon, allowing expression to be driven from the endogenous Para promoter.

In some embodiments, a Lad promoter and IPTG induction are used in this system (in lieu of Para and arabinose induction). In some embodiments, a rhamnose inducible promoter is used in this system. In some embodiments, a temperature sensitive promoter is used to drive expression of FNRS24Y.

Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in one or more gene(s) required for cell survival and/or growth.

In some embodiments, the bacterial cell comprises a genetic mutation in one or more endogenous gene(s) encoding a branched chain amino acid biosynthesis gene, wherein the genetic mutation reduces biosynthesis of one or more branched chain amino acids in the bacterial cell. In some embodiments, the endogenous gene encoding a branched chain amino acid biosynthesis gene is a keto acid reductoisomerase gene. Keto acid reductoisomerase gene is required for branched chain amino acid synthesis. Knock-out of this gene creates an auxotroph and requires the cell to import leucine to survive. In some embodiments, the bacterial cell comprises a genetic mutation in ilvC gene.

In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria.

Table 9 lists depicts exemplary bacterial genes which may be disrupted or deleted to produce an auxotrophic strain. These include, but are not limited to, genes required for oligonucleotide synthesis, amino acid synthesis, and cell wall synthesis.

TABLE 9
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Table 10 shows the survival of various amino acid auxotrophs in the mouse gut, as detected 24 hrs and 48 hrs post-gavage. These auxotrophs were generated using BW25113, a non-Nissle strain of E. coli.

TABLE 10
Survival of amino acid auxotrophs in the mouse gut

| Gene | AA Auxotroph | Pre-Gavage | 24 hours | 48 hours |
|---|---|---|---|---|
| argA | Arginine | Present | Present | Absent |
| cysE | Cysteine | Present | Present | Absent |
| glnA | Glutamine | Present | Present | Absent |
| glyA | Glycine | Present | Present | Absent |
| hisB | Histidine | Present | Present | Present |
| ilvA | Isoleucine | Present | Present | Absent |
| leuB | Leucine | Present | Present | Absent |
| lysA | Lysine | Present | Present | Absent |
| metA | Methionine | Present | Present | Present |
| pheA | Phenylalanine | Present | Present | Present |
| proA | Proline | Present | Present | Absent |
| serA | Serine | Present | Present | Present |
| thrC | Threonine | Present | Present | Present |
| trpC | Tryptophan | Present | Present | Present |
| tyrA | Tyrosine | Present | Present | Present |
| ilvD | Valine/Isoleucine/Leucine | Present | Present | Absent |
| thyA | Thiamine | Present | Absent | Absent |
| uraA | Uracil | Present | Absent | Absent |
| flhD | FlhD | Present | Present | Present |

For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, mc, ftsB, eno, pyrG, chpR, lgt, fbaA, pgk, yqgD, metK, yqgF, plsC, ygiT, pare, ribB, cca, ygjD, tdcF, yraL, yihA, ftsN, mud, murB, birA, secE, nusG, rplJ, rplL, rpoB, rpoC, ubiA, plsB, lexA, dnaB, ssb, alsK, groS, psd, om, yjeE, rpsR, chpS, ppa, valS, yjgP, yjgQ, dnaC, ribF, lspA, ispH, dapB, folA, imp, yabQ, ftsL, ftsl, murE, murF, mraY, murD, ftsW, murG, murC, ftsQ, ftsA, ftsZ, lpxC, secM, secA, can, folK, hemL, yadR, dapD, map, rpsB, infB, nusA, ftsH, obgE, rpmA, rplU, ispB, murA, yrbB, yrbK, yhbN, rpsl, rplM, degS, mreD, mreC, mreB, accB, accC, yrdC, clef, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsD, fusA, rpsM, ipsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplQ, rpmD, rpsE, rplR, rplF, rpsH, rpsK, rplE, rplX, rplN, rpsQ, ipmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, lnt, glnS, fidA, cydA, infA, cydC, ftsK, lolA, serS, rpsA, msbA, lpxK, kdsB, mukF, mukE, mukB, asnS, fabA, mviN, me, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabI, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain, “ACS Synthetic Biology (2015) DOI: 10.1021 lacssynbio.5b00085, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, I49G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the genetically engineered bacterium is a conditional auxotroph whose essential gene(s) is replaced using the arabinose system described herein.

In some embodiments, the genetically engineered bacterium of the disclosure is an auxotroph and also comprises kill-switch circuitry, such as any of the kill-switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA or ilvC, and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synthetic Biology (2015) 4: 307-16, the entire contents of which are expressly incorporated herein by reference). In some embodiments, the genetically engineered bacterium of the disclosure is an auxotroph and also comprises kill-switch circuitry, such as any of the kill-switch components and systems described herein, as well as another biosecurity system, such a conditional origin of replication (see Wright et al., supra).

In one embodiment, a genetically engineered bacterium, comprises one or more biosafety constructs integrated into the bacterial chromosome in combination with one or more biosafety plasmid(s). In some embodiments, the plasmid comprises a conditional origin of replication (COR), for which the plasmid replication initiator protein is provided in trans, i.e., is encoded by the chromosomally integrated biosafety construct. In some embodiments, the chromosomally integrated construct is further introduced into the host such that an auxotrophy results (e.g., dapA or thyA auxotrophy), which in turn is complemented by a gene product expressed from the biosafety plasmid construct. In some embodiments, the biosafety plasmid further encodes a broad-spectrum toxin (e.g., Kis), while the integrated biosafety construct encodes an anti-toxin (e.g., anti-Kis), permitting propagation of the plasmid in the bacterial cell containing both constructs. Without wishing to be bound by theory, this mechanism functions to select against plasmid spread by making the plasmid DNA itself disadvantageous to maintain by a wild-type bacterium. A non-limiting example of such a biosafety system is shown in FIG. 67A, FIG. 67B, FIG. 67C, and FIG. 67D.

Exemplary strains of the disclosure using this system are as follows. In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the dapA locus on the bacterial chromosome (low copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-adh2-brnQ driven by an inducible or constitutive promoter described herein, e.g., an FNRS promoter described herein (see, e.g., FIG. 55C).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the dapA locus on the bacterial chromosome (low copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-adh2-brnQ driven by a tet promoter (see, e.g., FIG. 54C).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the dapA locus on the bacterial chromosome (low copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-padA-brnQ driven by a tet promoter (see, e.g., FIG. 54D).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the dapA locus on the bacterial chromosome (low copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-padA-brnQ driven by an inducible or constitutive promoter described herein, e.g., an FNRS promoter described herein.

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the dapA locus on the bacterial chromosome (low copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-yqhD-brnQ driven by a tet promoter (see, e.g., FIG. 54E).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the dapA locus on the bacterial chromosome (low copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-yqhD-brnQ driven by an inducible or constitutive promoter described herein, e.g., an FNRS promoter described herein.

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the ThyA locus on the bacterial chromosome (low copy RBS; ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-adh2-brnQ driven by an inducible or constitutive promoter described herein, e.g., an FNRS promoter described herein (see, e.g., FIG. 55C).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the ThyA locus on the bacterial chromosome (low copy RBS; ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-adh2-brnQ driven by a tet promoter (see, e.g., FIG. 54C).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the ThyA locus on the bacterial chromosome (low copy RBS; ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-padA-brnQ driven by a tet promoter (see, e.g., FIG. 54D).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the ThyA locus on the bacterial chromosome (low copy RBS; ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-padA-brnQ driven by an inducible or constitutive promoter described herein, e.g., an FNRS promoter described herein.

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the ThyA locus on the bacterial chromosome (low copy RBS; ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-yqhD-brnQ driven by a tet promoter (see, e.g., FIG. 54E).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67C knocked into the ThyA locus on the bacterial chromosome (low copy RBS; ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-yqhD-brnQ driven by an inducible or constitutive promoter described herein, e.g., an FNRS promoter described herein.

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the dapA locus on the bacterial chromosome (medium copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-adh2-brnQ driven by an inducible or constitutive promoter described herein, e.g., an FNRS promoter described herein (see, e.g., FIG. 55C).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the dapA locus on the bacterial chromosome (medium copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-adh2-brnQ driven by a tet promoter (see, e.g., FIG. 54C).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the dapA locus on the bacterial chromosome (medium copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-padA-brnQ driven by a tet promoter (see, e.g., FIG. 54D).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the dapA locus on the bacterial chromosome (medium copy RBS); dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-padA-brnQ driven by an inducible or constitutive promoter described herein, e.g., an FNRS promoter described herein.

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the dapA locus on the bacterial chromosome (medium copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-yqhD-brnQ driven by a tet promoter (see, e.g., FIG. 54E).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the dapA locus on the bacterial chromosome (medium copy RBS; dapA::constitutive prom1 (BBA_J26700)-Pi(R6K)-constitutive promoter 2(P1)-Kis antitoxin). The strain further comprises a plasmid shown in FIG. 67A, except that the bla gene is replaced with a construct comprising codon optimized LeuDH-kivD-yqhD-brnQ driven by an inducible or constitutive promoter described herein, e.g., an FNRS promoter described herein.

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the ThyA locus on the bacterial chromosome (medium copy RBS); ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-adh2-brnQ driven by an inducible or constitutive promoter described herein, e.g., an FNRS promoter described herein (see, e.g., FIG. 55C).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the ThyA locus on the bacterial chromosome (medium copy RBS); ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-adh2-brnQ driven by a tet promoter (see, e.g., FIG. 54C).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the ThyA locus on the bacterial chromosome (medium copy RBS); ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-padA-brnQ driven by a tet promoter (see, e.g., FIG. 54D).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the ThyA locus on the bacterial chromosome (medium copy RBS; ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-padA-brnQ driven by an inducible or constitutive promoter described herein, e.g., an FNRS promoter described herein.

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the ThyA locus on the bacterial chromosome (medium copy RBS); ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-yqhD-brnQ driven by a tet promoter (see, e.g., FIG. 54E).

In one embodiment, the genetically engineered bacterium comprises ΔleuE, ΔilvC, and an inducible livKHMGF construct, e.g., a tet inducible livKHMGF construct or a FNR driven livKHMGF construct or a constitutively expressed livKHMGF construct, integrated into the bacterial chromosome, e.g., at the LacZ locus, and a construct shown in FIG. 67D knocked into the ThyA locus on the bacterial chromosome (medium copy RBS); ThyA::constitutive prom1 (BBA_J26700)-Pi(R6K)-constitutive promoter 2(P1)-Kis antitoxin). The strain further comprises a plasmid shown in FIG. 67B, except that the bla gene is replaced with a construct comprising codon optimized LeuDH-kivD-yqhD-brnQ driven by an inducible or constitutive promoter described herein, e.g., an FNRS promoter described herein.

Genetic Regulatory Circuits

In some embodiments, the genetically engineered bacteria comprise multi-layered genetic regulatory circuits for expressing the constructs described herein (see, e.g., U.S. Provisional Application No. 62/184,811, incorporated herein by reference in its entirety). The genetic regulatory circuits are useful to screen for mutant bacteria that produce a branched chain amino acid catabolism enzyme, BCAA transporter, and/or BCAA binding protein or rescue an auxotroph. In certain embodiments, the invention provides methods for selecting genetically engineered bacteria that produce one or more genes of interest.

In some embodiments, the invention provides genetically engineered bacteria comprising a gene or gene cassette for producing a payload and a T7 polymerase-regulated genetic regulatory circuit. For example, the genetically engineered bacteria comprise a first gene encoding a T7 polymerase, wherein the first gene is operably linked to a fumarate and nitrate reductase regulator (FNR)-responsive promoter; a second gene or gene cassette for producing a payload, wherein the second gene or gene cassette is operably linked to a T7 promoter that is induced by the T7 polymerase; and a third gene encoding an inhibitory factor, lysY, that is capable of inhibiting the T7 polymerase. In the presence of oxygen, FNR does not bind the FNR-responsive promoter, and the payload is not expressed. LysY is expressed constitutively (P-lac constitutive) and further inhibits T7 polymerase. In the absence of oxygen, FNR dimerizes and binds to the FNR-responsive promoter, T7 polymerase is expressed at a level sufficient to overcome lysY inhibition, and the payload is expressed. In some embodiments, the lysY gene is operably linked to an additional FNR binding site. In the absence of oxygen, FNR dimerizes to activate T7 polymerase expression as described above, and also inhibits lysY expression.

In some embodiments, the invention provides genetically engineered bacteria comprising a gene or gene cassette for producing a payload and a protease-regulated genetic regulatory circuit. For example, the genetically engineered bacteria comprise a first gene encoding an mf-1on protease, wherein the first gene is operably linked to a FNR-responsive promoter; a second gene or gene cassette for producing a payload operably linked to a tet regulatory region (tetO); and a third gene encoding an mf-1on degradation signal linked to a tet repressor (tetR), wherein the tetR is capable of binding to the tet regulatory region and repressing expression of the second gene or gene cassette. The mf-1on protease is capable of recognizing the mf-1on degradation signal and degrading the tetR. In the presence of oxygen, FNR does not bind the FNR-responsive promoter, the repressor is not degraded, and the payload is not expressed. In the absence of oxygen, FNR dimerizes and binds the FNR-responsive promoter, thereby inducing expression of mf-1on protease. The mf-1on protease recognizes the mf-1on degradation signal and degrades the tetR, and the payload is expressed.

In some embodiments, the invention provides genetically engineered bacteria comprising a gene or gene cassette for producing a payload and a repressor-regulated genetic regulatory circuit. For example, the genetically engineered bacteria comprise a first gene encoding a first repressor, wherein the first gene is operably linked to a FNR-responsive promoter; a second gene or gene cassette for producing a payload operably linked to a first regulatory region comprising a constitutive promoter; and a third gene encoding a second repressor, wherein the second repressor is capable of binding to the first regulatory region and repressing expression of the second gene or gene cassette. The third gene is operably linked to a second regulatory region comprising a constitutive promoter, wherein the first repressor is capable of binding to the second regulatory region and inhibiting expression of the second repressor. In the presence of oxygen, FNR does not bind the FNR-responsive promoter, the first repressor is not expressed, the second repressor is expressed, and the payload is not expressed. In the absence of oxygen, FNR dimerizes and binds the FNR-responsive promoter, the first repressor is expressed, the second repressor is not expressed, and the payload is expressed.

Examples of repressors useful in these embodiments include, but are not limited to, ArgR, TetR, ArsR, AscG, LacI, CscR, DeoR, DgoR, FruR, GalR, GatR, CI, LexA, RafR, QacR, and PtxS (US20030166191).

In some embodiments, the invention provides genetically engineered bacteria comprising a gene or gene cassette for producing a payload and a regulatory RNA-regulated genetic regulatory circuit. For example, the genetically engineered bacteria comprise a first gene encoding a regulatory RNA, wherein the first gene is operably linked to a FNR-responsive promoter, and a second gene or gene cassette for producing a payload. The second gene or gene cassette is operably linked to a constitutive promoter and further linked to a nucleotide sequence capable of producing an mRNA hairpin that inhibits translation of the payload. The regulatory RNA is capable of eliminating the mRNA hairpin and inducing payload translation via the ribosomal binding site. In the presence of oxygen, FNR does not bind the FNR-responsive promoter, the regulatory RNA is not expressed, and the mRNA hairpin prevents the payload from being translated. In the absence of oxygen, FNR dimerizes and binds the FNR-responsive promoter, the regulatory RNA is expressed, the mRNA hairpin is eliminated, and the payload is expressed.

In some embodiments, the invention provides genetically engineered bacteria comprising a gene or gene cassette for producing a payload and a CRISPR-regulated genetic regulatory circuit. For example, the genetically engineered bacteria comprise a Cas9 protein; a first gene encoding a CRISPR guide RNA, wherein the first gene is operably linked to a FNR-responsive promoter; a second gene or gene cassette for producing a payload, wherein the second gene or gene cassette is operably linked to a regulatory region comprising a constitutive promoter; and a third gene encoding a repressor operably linked to a constitutive promoter, wherein the repressor is capable of binding to the regulatory region and repressing expression of the second gene or gene cassette. The third gene is further linked to a CRISPR target sequence that is capable of binding to the CRISPR guide RNA, wherein said binding to the CRISPR guide RNA induces cleavage by the Cas9 protein and inhibits expression of the repressor. In the presence of oxygen, FNR does not bind the FNR-responsive promoter, the guide RNA is not expressed, the repressor is expressed, and the payload is not expressed. In the absence of oxygen, FNR dimerizes and binds the FNR-responsive promoter, the guide RNA is expressed, the repressor is not expressed, and the payload is expressed.

In some embodiments, the invention provides genetically engineered bacteria comprising a gene or gene cassette for producing a payload and a recombinase-regulated genetic regulatory circuit. For example, the genetically engineered bacteria comprise a first gene encoding a recombinase, wherein the first gene is operably linked to a FNR-responsive promoter, and a second gene or gene cassette for producing a payload operably linked to a constitutive promoter. The second gene or gene cassette is inverted in orientation (3′ to 5′) and flanked by recombinase binding sites, and the recombinase is capable of binding to the recombinase binding sites to induce expression of the second gene or gene cassette by reverting its orientation (5′ to 3′). In the presence of oxygen, FNR does not bind the FNR-responsive promoter, the recombinase is not expressed, the payload remains in the 3′ to 5′ orientation, and no functional payload is produced. In the absence of oxygen, FNR dimerizes and binds the FNR-responsive promoter, the recombinase is expressed, the payload is reverted to the 5′ to 3′ orientation, and functional payload is produced.

In some embodiments, the invention provides genetically engineered bacteria comprising a gene or gene cassette for producing a payload and a polymerase- and recombinase-regulated genetic regulatory circuit. For example, the genetically engineered bacteria comprise a first gene encoding a recombinase, wherein the first gene is operably linked to a FNR-responsive promoter; a second gene or gene cassette for producing a payload operably linked to a T7 promoter; a third gene encoding a T7 polymerase, wherein the T7 polymerase is capable of binding to the T7 promoter and inducing expression of the payload. The third gene encoding the T7 polymerase is inverted in orientation (3′ to 5′) and flanked by recombinase binding sites, and the recombinase is capable of binding to the recombinase binding sites to induce expression of the T7 polymerase gene by reverting its orientation (5′ to 3′). In the presence of oxygen, FNR does not bind the FNR-responsive promoter, the recombinase is not expressed, the T7 polymerase gene remains in the 3′ to 5′ orientation, and the payload is not expressed. In the absence of oxygen, FNR dimerizes and binds the FNR-responsive promoter, the recombinase is expressed, the T7 polymerase gene is reverted to the 5′ to 3′ orientation, and the payload is expressed.

Kill Switches

In some embodiments, the genetically engineered bacteria also comprise a kill switch (see, e.g., U.S. Provisional Application Nos. 62/183,935 and 62/263,329, each of which are expressly incorporated herein by reference in their entireties). The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death.

Bacteria engineered with kill switches have been engineered for in vitro research purposes, e.g., to limit the spread of a biofuel-producing microorganism outside of a laboratory environment. Bacteria engineered for in vivo administration to treat a disease or disorder may also be programmed to die at a specific time after the expression and delivery of a heterologous gene or genes, for example, a therapeutic gene(s) or after the subject has experienced the therapeutic effect. For example, in some embodiments, the kill switch is activated to kill the bacteria after a period of time following expression of an amino acid catabolism enzyme. In some embodiments, the kill switch is activated in a delayed fashion following expression of the amino acid catabolism gene, for example, after the production of the amino acid catabolism enzyme. Alternatively, the bacteria may be engineered to die after the bacteria has spread outside of a disease site. Specifically, it may be useful to prevent long-term colonization of subjects by the microorganism, spread of the microorganism outside the area of interest (for example, outside the gut) within the subject, or spread of the microorganism outside of the subject into the environment (for example, spread to the environment through the stool of the subject).

Examples of such toxins that can be used in kill-switches include, but are not limited to, bacteriocins, lysins, and other molecules that cause cell death by lysing cell membranes, degrading cellular DNA, or other mechanisms. Such toxins can be used individually or in combination. The switches that control their production can be based on, for example, transcriptional activation (toggle switches; see, e.g., Gardner et al., 2000), translation (riboregulators), or DNA recombination (recombinase-based switches), and can sense environmental stimuli such as anaerobiosis or reactive oxygen species. These switches can be activated by a single environmental factor or may require several activators in AND, OR, NAND and NOR logic configurations to induce cell death. For example, an AND riboregulator switch is activated by tetracycline, isopropyl β-D-1-thiogalactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death. Examples of kill switches are known in the art (Callura et al., 2010). In some embodiments, the kill switch is activated to kill the bacteria after a period of time following oxygen level-dependent expression of an amino acid catabolism enzyme. In some embodiments, the kill switch is activated in a delayed fashion following oxygen level-dependent expression of an amino acid catabolism enzyme.

Kill-switches can be designed such that a toxin is produced in response to an environmental condition or external signal (e.g., the bacteria is killed in response to an external cue; i.e., an activation-based kill switch, see FIG. 34-37) or, alternatively designed such that a toxin is produced once an environmental condition no longer exists or an external signal is ceased (i.e., a repression-based kill switch, see FIGS. 38-42).

Thus, in some embodiments, the genetically engineered bacteria of the disclosure are further programmed to die after sensing an exogenous environmental signal, for example, in a low oxygen environment. In some embodiments, the genetically engineered bacteria of the present disclosure, e.g., bacteria expressing an amino acid catabolism enzyme, comprise one or more genes encoding one or more recombinase(s), whose expression is induced in response to an environmental condition or signal and causes one or more recombination events that ultimately leads to the expression of a toxin which kills the cell. In some embodiments, the at least one recombination event is the flipping of an inverted heterologous gene encoding a bacterial toxin which is then constitutively expressed after it is flipped by the first recombinase. In one embodiment, constitutive expression of the bacterial toxin kills the genetically engineered bacterium. In these types of kill-switch systems once the engineered bacterial cell senses the exogenous environmental condition and expresses the heterologous gene of interest, the recombinant bacterial cell is no longer viable.

In another embodiment in which the genetically engineered bacteria of the present disclosure, e.g., bacteria expressing an amino acid catabolism enzyme, express one or more recombinase(s) in response to an environmental condition or signal causing at least one recombination event, the genetically engineered bacterium further expresses a heterologous gene encoding an anti-toxin in response to an exogenous environmental condition or signal. In one embodiment, the at least one recombination event is flipping of an inverted heterologous gene encoding a bacterial toxin by a first recombinase. In one embodiment, the inverted heterologous gene encoding the bacterial toxin is located between a first forward recombinase recognition sequence and a first reverse recombinase recognition sequence. In one embodiment, the heterologous gene encoding the bacterial toxin is constitutively expressed after it is flipped by the first recombinase. In one embodiment, the anti-toxin inhibits the activity of the toxin, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In another embodiment, the at least one recombination event is flipping of an inverted heterologous gene encoding a second recombinase by a first recombinase, followed by the flipping of an inverted heterologous gene encoding a bacterial toxin by the second recombinase. In one embodiment, the inverted heterologous gene encoding the second recombinase is located between a first forward recombinase recognition sequence and a first reverse recombinase recognition sequence. In one embodiment, the inverted heterologous gene encoding the bacterial toxin is located between a second forward recombinase recognition sequence and a second reverse recombinase recognition sequence. In one embodiment, the heterologous gene encoding the second recombinase is constitutively expressed after it is flipped by the first recombinase. In one embodiment, the heterologous gene encoding the bacterial toxin is constitutively expressed after it is flipped by the second recombinase. In one embodiment, the genetically engineered bacterium is killed by the bacterial toxin. In one embodiment, the genetically engineered bacterium further expresses a heterologous gene encoding an anti-toxin in response to the exogenous environmental condition. In one embodiment, the anti-toxin inhibits the activity of the toxin when the exogenous environmental condition is present, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In one embodiment, the at least one recombination event is flipping of an inverted heterologous gene encoding a second recombinase by a first recombinase, followed by flipping of an inverted heterologous gene encoding a third recombinase by the second recombinase, followed by flipping of an inverted heterologous gene encoding a bacterial toxin by the third recombinase. Accordingly, in one embodiment, the disclosure provides at least 1, 2, 3, 4, 5, 6, 7, 8, 9, 10, 11, 12, 13, 14, 15, 16, 17, 18, 19, or 20 recombinases that can be used serially.

In one embodiment, the at least one recombination event is flipping of an inverted heterologous gene encoding a first excision enzyme by a first recombinase. In one embodiment, the inverted heterologous gene encoding the first excision enzyme is located between a first forward recombinase recognition sequence and a first reverse recombinase recognition sequence. In one embodiment, the heterologous gene encoding the first excision enzyme is constitutively expressed after it is flipped by the first recombinase. In one embodiment, the first excision enzyme excises a first essential gene. In one embodiment, the programmed recombinant bacterial cell is not viable after the first essential gene is excised.

In one embodiment, the first recombinase further flips an inverted heterologous gene encoding a second excision enzyme. In one embodiment, the wherein the inverted heterologous gene encoding the second excision enzyme is located between a second forward recombinase recognition sequence and a second reverse recombinase recognition sequence. In one embodiment, the heterologous gene encoding the second excision enzyme is constitutively expressed after it is flipped by the first recombinase. In one embodiment, the genetically engineered bacterium dies or is no longer viable when the first essential gene and the second essential gene are both excised. In one embodiment, the genetically engineered bacterium dies or is no longer viable when either the first essential gene is excised or the second essential gene is excised by the first recombinase.

In one embodiment, the first excision enzyme is Xis1. In one embodiment, the first excision enzyme is Xis2. In one embodiment, the first excision enzyme is Xis1, and the second excision enzyme is Xis2.

In one embodiment, the genetically engineered bacterium dies after the at least one recombination event occurs. In another embodiment, the genetically engineered bacterium is no longer viable after the at least one recombination event occurs.

In any of these embodiment, the recombinase can be a recombinase selected from the group consisting of: Bxb1, PhiC31, TP901, Bxb1, PhiC31, TP901, HK022, HP1, R4, Int1, Int2, Int3, Int4, Int5, Int6, Int1, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34, or a biologically active fragment thereof.

In the above-described kill-switch circuits, a toxin is produced in the presence of an environmental factor or signal. In another aspect of kill-switch circuitry, a toxin may be repressed in the presence of an environmental factor (not produced) and then produced once the environmental condition or external signal is no longer present. Such kill switches are called repression-based kill switches and represent systems in which the bacterial cells are viable only in the presence of an external factor or signal, such as arabinose or other sugar. Exemplary kill switch designs in which the toxin is repressed in the presence of an external factor or signal (and activated once the external signal is removed) is shown in FIGS. 67-71. The disclosure provides recombinant bacterial cells which express one or more heterologous gene(s) upon sensing arabinose or other sugar in the exogenous environment. In this aspect, the recombinant bacterial cells contain the araC gene, which encodes the AraC transcription factor, as well as one or more genes under the control of the araBAD promoter. In the absence of arabinose, the AraC transcription factor adopts a conformation that represses transcription of genes under the control of the araBAD promoter. In the presence of arabinose, the AraC transcription factor undergoes a conformational change that allows it to bind to and activate the araBAD promoter, which induces expression of the desired gene, for example tetR, which represses expression of a toxin gene. In this embodiment, the toxing gene is repressed in the presence of arabinose or other sugar. In an environment where arabinose is not present, the tetR gene is not activated and the toxin is expressed, thereby killing the bacteria. The arabinose system can also be used to express an essential gene, in which the essential gene is only expressed in the presence of arabinose or other sugar and is not expressed when arabinose or other sugar is absent from the environment.

Thus, in some embodiments in which one or more heterologous gene(s) are expressed upon sensing arabinose in the exogenous environment, the one or more heterologous genes are directly or indirectly under the control of the araBAD promoter. In some embodiments, the expressed heterologous gene is selected from one or more of the following: a heterologous therapeutic gene, a heterologous gene encoding an antitoxin, a heterologous gene encoding a repressor protein or polypeptide, for example, a TetR repressor, a heterologous gene encoding an essential protein not found in the bacterial cell, and/or a heterologous encoding a regulatory protein or polypeptide.

Arabinose inducible promoters are known in the art, including Pₐᵣₐ, P_{araB}, P_{araC}, and P_{araBAD}. In one embodiment, the arabinose inducible promoter is from E. coli. In some embodiments, the P_{araC} promoter and the P_{araBAD} promoter operate as a bidirectional promoter, with the P_{araBAD} promoter controlling expression of a heterologous gene(s) in one direction, and the P_{araC} (in close proximity to, and on the opposite strand from the P_{araBAD} promoter), controlling expression of a heterologous gene(s) in the other direction. In the presence of arabinose, transcription of both heterologous genes from both promoters is induced. However, in the absence of arabinose, transcription of both heterologous genes from both promoters is not induced.

In one exemplary embodiment of the disclosure, the engineered bacteria of the present disclosure contains a kill-switch having at least the following sequences: a P_{araBAD} promoter operably linked to a heterologous gene encoding a Tetracycline Repressor Protein (TetR), a P_{araC} promoter operably linked to a heterologous gene encoding AraC transcription factor, and a heterologous gene encoding a bacterial toxin operably linked to a promoter which is repressed by the Tetracycline Repressor Protein (P_{TetR}). In the presence of arabinose, the AraC transcription factor activates the P_{araBAD} promoter, which activates transcription of the TetR protein which, in turn, represses transcription of the toxin. In the absence of arabinose, however, AraC suppresses transcription from the P_{araBAD} promoter and no TetR protein is expressed. In this case, expression of the heterologous toxin gene is activated, and the toxin is expressed. The toxin builds up in the recombinant bacterial cell, and the recombinant bacterial cell is killed. In one embodiment, the araC gene encoding the AraC transcription factor is under the control of a constitutive promoter and is therefore constitutively expressed.

In one embodiment of the disclosure, the recombinant bacterial cell further comprises an antitoxin under the control of a constitutive promoter. In this situation, in the presence of arabinose, the toxin is not expressed due to repression by TetR protein, and the antitoxin protein builds-up in the cell. However, in the absence of arabinose, TetR protein is not expressed, and expression of the toxin is induced. The toxin begins to build-up within the recombinant bacterial cell. The recombinant bacterial cell is no longer viable once the toxin protein is present at either equal or greater amounts than that of the anti-toxin protein in the cell, and the recombinant bacterial cell will be killed by the toxin.

In another embodiment of the disclosure, the recombinant bacterial cell further comprises an antitoxin under the control of the P_{araBAD} promoter. In this situation, in the presence of arabinose, TetR and the anti-toxin are expressed, the anti-toxin builds up in the cell, and the toxin is not expressed due to repression by TetR protein. However, in the absence of arabinose, both the TetR protein and the anti-toxin are not expressed, and expression of the toxin is induced. The toxin begins to build-up within the recombinant bacterial cell. The recombinant bacterial cell is no longer viable once the toxin protein is expressed, and the recombinant bacterial cell will be killed by the toxin.

In another exemplary embodiment of the disclosure, the engineered bacteria of the present disclosure contains a kill-switch having at least the following sequences: a P_{araBAD} promoter operably linked to a heterologous gene encoding an essential polypeptide not found in the recombinant bacterial cell (and required for survival), and a P_{araC} promoter operably linked to a heterologous gene encoding AraC transcription factor. In the presence of arabinose, the AraC transcription factor activates the P_{araBAD} promoter, which activates transcription of the heterologous gene encoding the essential polypeptide, allowing the recombinant bacterial cell to survive. In the absence of arabinose, however, AraC suppresses transcription from the P_{araBAD} promoter and the essential protein required for survival is not expressed. In this case, the recombinant bacterial cell dies in the absence of arabinose. In some embodiments, the sequence of P_{araBAD} promoter operably linked to a heterologous gene encoding an essential polypeptide not found in the recombinant bacterial cell can be present in the bacterial cell in conjunction with the TetR/toxin kill-switch system described directly above. In some embodiments, the sequence of P_{araBAD} promoter operably linked to a heterologous gene encoding an essential polypeptide not found in the recombinant bacterial cell can be present in the bacterial cell in conjunction with the TetR/toxin/anti-toxin kill-switch system described directly above.

In yet other embodiments, the bacteria may comprise a plasmid stability system with a plasmid that produces both a short-lived anti-toxin and a long-lived toxin. In this system, the bacterial cell produces equal amounts of toxin and anti-toxin to neutralize the toxin. However, if/when the cell loses the plasmid, the short-lived anti-toxin begins to decay. When the anti-toxin decays completely the cell dies as a result of the longer-lived toxin killing it.

In some embodiments, the engineered bacteria of the present disclosure, for example, bacteria expressing an amino acid catabolism enzyme further comprise the gene(s) encoding the components of any of the above-described kill-switch circuits.

In any of the above-described embodiments, the bacterial toxin is selected from the group consisting of a lysin, Hok, Fst, TisB, LdrD, Kid, SymE, MazF, FlmA, Ibs, XCV2162, dinJ, CcdB, MazF, ParE, YafO, Zeta, hicB, relB, yhaV, yoeB, chpBK, hipA, microcin B, microcin B17, microcin C, microcin C7-051, microcin J25, microcin ColV, microcin 24, microcin L, microcin D93, microcin L, microcin E492, microcin H47, microcin 147, microcin M, colicin A, colicin E1, colicin K, colicin N, colicin U, colicin B, colicin Ia, colicin Ib, colicin 5, colicin10, colicin S4, colicin Y, colicin E2, colicin E7, colicin E8, colicin E9, colicin E3, colicin E4, colicin E6; colicin E5, colicin D, colicin M, and cloacin DF13, or a biologically active fragment thereof.

In any of the above-described embodiments, the anti-toxin is selected from the group consisting of an anti-lysin, Sok, RNAII, IstR, Rd1D, Kis, SymR, MazE, FlmB, Sib, ptaRNA1, yafQ, CcdA, MazE, ParD, yafN, Epsilon, HicA, relE, prlF, yefM, chpBI, hipB, MccE, MccE^{cTD}, MccF, Cai, ImmEI, Cki, Cni, Cui, Cbi, Iia, Imm, Cfi, Im10, Csi, Cyi, Im2, Im7, Im8, Im9, Im3, Im4, ImmE6, cloacin immunity protein (Cim), ImmES, ImmD, and Cmi, or a biologically active fragment thereof.

In one embodiment, the bacterial toxin is bactericidal to the genetically engineered bacterium. In one embodiment, the bacterial toxin is bacteriostatic to the genetically engineered bacterium.

In one embodiment, the method further comprises administering a second recombinant bacterial cell to the subject, wherein the second recombinant bacterial cell comprises a heterologous reporter gene operably linked to an inducible promoter that is directly or indirectly induced by an exogenous environmental condition. In one embodiment, the heterologous reporter gene is a fluorescence gene. In one embodiment, the fluorescence gene encodes a green fluorescence protein (GFP). In another embodiment, the method further comprises administering a second recombinant bacterial cell to the subject, wherein the second recombinant bacterial cell expresses a lacZ reporter construct that cleaves a substrate to produce a small molecule that can be detected in urine (see, for example, Danio et al., Science Translational Medicine, 7(289):1-12, 2015, the entire contents of which are expressly incorporated herein by reference).

Isolated Plasmids

In other embodiments, the disclosure provides an isolated plasmid comprising a first nucleic acid encoding a first payload operably linked to a first inducible promoter, and a second nucleic acid encoding a second payload operably linked to a second inducible promoter. In other embodiments, the disclosure provides an isolated plasmid further comprising a third nucleic acid encoding a third payload operably linked to a third inducible promoter. In other embodiments, the disclosure provides a plasmid comprising four, five, six, or more nucleic acids encoding four, five, six, or more payloads operably linked to inducible promoters. In any of the embodiments described here, the first, second, third, fourth, fifth, sixth, etc “payload(s)” can be a branched chain amino acid catabolism enzyme, a transporter of branched chain amino acids, a binding protein of branched chain amino acids, or other sequence described herein. In one embodiment, the nucleic acid encoding the first payload and the nucleic acid encoding the second payload are operably linked to the first inducible promoter. In one embodiment, the nucleic acid encoding the first payload is operably linked to a first inducible promoter and the nucleic acid encoding the second payload is operably linked to a second inducible promoter. In one embodiment, the first inducible promoter and the second inducible promoter are separate copies of the same inducible promoter. In another embodiment, the first inducible promoter and the second inducible promoter are different inducible promoters. In other embodiments comprising a third nucleic acid, the nucleic acid encoding the third payload and the nucleic acid encoding the first and second payloads are all operably linked to the same inducible promoter. In other embodiments, the nucleic acid encoding the first payload is operably linked to a first inducible promoter, the nucleic acid encoding the second payload is operably linked to a second inducible promoter, and the nucleic acid encoding to third payload is operably linked to a third inducible promoter. In some embodiments, the first, second, and third inducible promoters are separate copies of the same inducible promoter. In other embodiments, the first inducible promoter, the second inducible promoter, and the third inducible promoter are different inducible promoters. In some embodiments, the first promoter, the second promoter, and the optional third promoter, or the first promoter and the second promoter and the optional third promoter, are each directly or indirectly induced by low-oxygen or anaerobic conditions. In other embodiments, the first promoter, the second promoter, and the optional third promoter, or the first promoter and the second promoter and the optional third promoter, are each a fumarate and nitrate reduction regulator (FNR) responsive promoter. In other embodiments, the first promoter, the second promoter, and the optional third promoter, or the first promoter and the second promoter and the optional third promoter are each a ROS-inducible regulatory region. In other embodiments, the first promoter, the second promoter, and the optional third promoter, or the first promoter and the second promoter and the optional third promoter are each a RNS-inducible regulatory region.

In some embodiments, the heterologous gene encoding a branched chain amino acid catabolism enzyme is operably linked to a constitutive promoter. In one embodiment, the constitutive promoter is a lac promoter. In another embodiment, the constitutive promoter is a tet promoter. In another embodiment, the constitutive promoter is a constitutive Escherichia coli σ³² promoter. In another embodiment, the constitutive promoter is a constitutive Escherichia coli σ⁷⁰ promoter. In another embodiment, the constitutive promoter is a constitutive Bacillus subtilis σ^{A} promoter. In another embodiment, the constitutive promoter is a constitutive Bacillus subtilis σ^{B} promoter. In another embodiment, the constitutive promoter is a Salmonella promoter. In other embodiments, the constitutive promoter is a bacteriophage T7 promoter. In other embodiments, the constitutive promoter is and a bacteriophage SP6 promoter. In any of the above-described embodiments, the plasmid further comprises a heterologous gene encoding a transporter of a branched chain amino acid, a BCAA binding protein, and/or a kill switch construct, which may be operably linked to a constitutive promoter or an inducible promoter.

In some embodiments, the isolated plasmid comprises at least one heterologous branched chain amino acid catabolism enzyme gene operably linked to a first inducible promoter; a heterologous gene encoding a TetR protein operably linked to a P_{araBAD} promoter, a heterologous gene encoding AraC operably linked to a P_{araC} promoter, a heterologous gene encoding an antitoxin operably linked to a constitutive promoter, and a heterologous gene encoding a toxin operably linked to a P_{TetR} promoter. In another embodiment, the isolated plasmid comprises at least one heterologous gene encoding a branched chain amino acid catabolism enzyme operably linked to a first inducible promoter; a heterologous gene encoding a TetR protein and an anti-toxin operably linked to a P_{araBAD} promoter, a heterologous gene encoding AraC operably linked to a P_{araC} promoter, and a heterologous gene encoding a toxin operably linked to a P_{TetR} promoter.

In some embodiments, a first nucleic acid encoding a branched chain amino acid catabolism enzyme comprises a kivD gene. In other embodiments, a first nucleic acid encoding a branched chain amino acid catabolism enzyme is a BCKD gene or a BCKD operon. In some embodiments, the kivD gene or BCKD operon is coexpressed with an additional branched chain amino acid dehydrogenase, e.g., a leucine dehydrogenase, e.g., leuDH, or a branched chain amino acid aminotransferase, e.g., ilvE or an amino acid oxidase, e.g., L-AAD. In other embodiments, a gene encoding an alcohol dehydrogenase, e.g., adh2 or yqhD, is further coexpressed. In other embodiments, a gene encoding an aldehyde dehydrogenase, e.g., padA, is further coexpressed.

In some embodiments, a second nucleic acid encoding a transporter of branched chain amino acids comprises a livKHMGF operon. In one embodiment, the livKHMGF operon is an Escherichia coli livKHMGF operon. In another embodiment, the livKHMGF operon has at least about 90% identity to the uppercase sequence of SEQ ID NO:5. In another embodiment, the livKHMGF operon comprises the uppercase sequence of SEQ ID NO:5. In another embodiment, the second nucleic acid encoding a transporter of branched chain amino acids comprises brnQ gene. In another embodiment, the brnQ gene has at least about 90% identity to the uppercase sequence of SEQ ID NO: 64. In another embodiment, the brnQ gene comprises the uppercase sequence of SEQ ID NO: 64.

In some embodiments, a third nucleic acid encoding a binding protein of branched chain amino acids comprises livJ gene. In another embodiment, the livJ gene has at least about 90% identity to the uppercase sequence of SEQ ID NO: 12. In another embodiment, the livJ gene comprises the uppercase sequence of SEQ ID NO: 12.

In one embodiment, the plasmid is a high-copy plasmid. In another embodiment, the plasmid is a low-copy plasmid.

In another aspect, the disclosure provides a recombinant bacterial cell comprising an isolated plasmid described herein. In another embodiment, the disclosure provides a pharmaceutical composition comprising the recombinant bacterial cell.

In one embodiment, the bacterial cell further comprises a genetic mutation in an endogenous gene encoding an exporter of a branched chain amino acid, wherein the genetic mutation reduces export of the branched chain amino acid from the bacterial cell. In one embodiment, the endogenous gene encoding an exporter of a branched chain amino acid is a leuE gene.

In one embodiment, the bacterial cell further comprises a genetic mutation in an endogenous gene encoding a branched chain amino acid biosynthesis gene, wherein the genetic mutation reduces biosynthesis of one or more branched chain amino acids in the bacterial cell. In one embodiment, the endogenous gene encoding a branched chain amino acid biosynthesis gene is an ilvC gene.

Host-Plasmid Mutual Dependency

In some embodiments, the genetically engineered bacteria also comprise a plasmid that has been modified to create a host-plasmid mutual dependency. In certain embodiments, the mutually dependent host-plasmid platform is GeneGuard (Wright et al., 2015). In some embodiments, the GeneGuard plasmid comprises (i) a conditional origin of replication, in which the requisite replication initiator protein is provided in trans; (ii) an auxotrophic modification that is rescued by the host via genomic translocation and is also compatible for use in rich media; and/or (iii) a nucleic acid sequence which encodes a broad-spectrum toxin. The toxin gene may be used to select against plasmid spread by making the plasmid DNA itself disadvantageous for strains not expressing the anti-toxin (e.g., a wild-type bacterium). In some embodiments, the GeneGuard plasmid is stable for at least 100 generations without antibiotic selection. In some embodiments, the GeneGuard plasmid does not disrupt growth of the host. The GeneGuard plasmid is used to greatly reduce unintentional plasmid propagation in the genetically engineered bacteria described herein.

The mutually dependent host-plasmid platform may be used alone or in combination with other biosafety mechanisms, such as those described herein (e.g., kill switches, auxotrophies). In some embodiments, the genetically engineered bacteria comprise a GeneGuard plasmid. In other embodiments, the genetically engineered bacteria comprise a GeneGuard plasmid and/or one or more kill switches. In other embodiments, the genetically engineered bacteria comprise a GeneGuard plasmid and/or one or more auxotrophies. In still other embodiments, the genetically engineered bacteria comprise a GeneGuard plasmid, one or more kill switches, and/or one or more auxotrophies.

In some embodiments, the vector comprises a conditional origin of replication. In some embodiments, the conditional origin of replication is a R6K or ColE2-P9. In embodiments where the plasmid comprises the conditional origin of replication R6K, the host cell expresses the replication initiator protein 7E. In embodiments where the plasmid comprises the conditional origin or replication ColE2, the host cell expresses the replication initiator protein RepA. It is understood by those of skill in the art that the expression of the replication initiator protein may be regulated so that a desired expression level of the protein is achieved in the host cell to thereby control the replication of the plasmid. For example, in some embodiments, the expression of the gene encoding the replication initiator protein may be placed under the control of a strong, moderate, or weak promoter to regulate the expression of the protein.

In some embodiments, the vector comprises a gene encoding a protein required for complementation of a host cell auxotrophy, preferably a rich-media compatible auxotrophy. In some embodiments, the host cell is auxotrophic for thymidine (ΔthyA), and the vector comprises the thymidylate synthase (thyA) gene. In some embodiments, the host cell is auxotrophic for diaminopimelic acid (ΔdapA) and the vector comprises the 4-hydroxy-tetrahydrodipicolinate synthase (dapA) gene. It is understood by those of skill in the art that the expression of the gene encoding a protein required for complementation of the host cell auxotrophy may be regulated so that a desired expression level of the protein is achieved in the host cell.

In some embodiments, the vector comprises a toxin gene. In some embodiments, the host cell comprises an anti-toxin gene encoding and/or required for the expression of an anti-toxin. In some embodiments, the toxin is Zeta and the anti-toxin is Epsilon. In some embodiments, the toxin is Kid, and the anti-toxin is Kis. In preferred embodiments, the toxin is bacteriostatic. Any of the toxin/antitoxin pairs described herein may be used in the vector systems of the present disclosure. It is understood by those of skill in the art that the expression of the gene encoding the toxin may be regulated using art known methods to prevent the expression levels of the toxin from being deleterious to a host cell that expresses the anti-toxin. For example, in some embodiments, the gene encoding the toxin may be regulated by a moderate promoter. In other embodiments, the gene encoding the toxin may be cloned adjacent to ribosomal binding site of interest to regulate the expression of the gene at desired levels (see, e.g., Wright et al. (2015)).

Integration

In some embodiments, any of the gene(s) or gene cassette(s) of the present disclosure may be integrated into the bacterial chromosome at one or more integration sites. One or more copies of the gene (for example, an amino acid catabolism gene, BCAA transporter gene, and/or BCAA binding protein gene) or gene cassette (for example, a gene cassette comprising an amino acid catabolism gene, an amino acid transporter gene, a BCAA binding protein gene) may be integrated into the bacterial chromosome. Having multiple copies of the gene or gene cassette integrated into the chromosome allows for greater production of the payload, e.g., amino acid catabolism enzyme, BCAA transporter gene, and/or BCAA binding protein gene and other enzymes of a gene cassette, and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions.

For example, FIG. 68B depicts a map of integration sites within the E. coli Nissle chromosome. FIG. 68B depicts three bacterial strains wherein the RFP gene has been successfully integrated into the bacterial chromosome at an integration site.

Secretion

In some embodiments, the genetically engineered bacteria further comprise a native secretion mechanism or non-native secretion mechanism that is capable of secreting a molecule from the bacterial cytoplasm in the extracellular environment. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane.

In Gram-negative bacteria, secretion machineries may span one or both of the inner and outer membranes. In some embodiments, the genetically engineered bacteria further comprise a non-native double membrane-spanning secretion system. Membrane-spanning secretion systems include, but are not limited to, the type I secretion system (T1SS), the type II secretion system (T2SS), the type III secretion system (T3SS), the type IV secretion system (T4SS), the type VI secretion system (T6SS), and the resistance-nodulation-division (RND) family of multi-drug efflux pumps (Pugsley 1993; Gerlach et al., 2007; Collinson et al., 2015; Costa et al., 2015; Reeves et al., 2015; WO2014138324A1, incorporated herein by reference). Examples of such secretion systems are shown in FIGS. 72, 73, 74, 75, 76, 77, and 78. Mycobacteria, which have a Gram-negative-like cell envelope, may also encode a type VII secretion system (T7SS) (Stanley et al., 2003). With the exception of the T2SS, double membrane-spanning secretions generally transport substrates from the bacterial cytoplasm directly into the extracellular space or into the target cell. In contrast, the T2SS and secretion systems that span only the outer membrane may use a two-step mechanism, wherein substrates are first translocated to the periplasm by inner membrane-spanning transporters, and then transferred to the outer membrane or secreted into the extracellular space. Outer membrane-spanning secretion systems include, but are not limited to, the type V secretion or autotransporter system or autosecreter system (TSSS), the curli secretion system, and the chaperone-usher pathway for pili assembly (Saier, 2006; Costa et al., 2015).

In some embodiments, the genetically engineered bacteria of the invention further comprise a type III or a type III-like secretion system (T3SS) from Shigella, Salmonella, E. coli, Bivrio, Burkholderia, Yersinia, Chlamydia, or Pseudomonas. The T3SS is capable of transporting a protein from the bacterial cytoplasm to the host cytoplasm through a needle complex. The T3SS may be modified to secrete the molecule from the bacterial cytoplasm, but not inject the molecule into the host cytoplasm. Thus, the molecule is secreted into the gut lumen or other extracellular space. In some embodiments, the genetically engineered bacteria comprise said modified T3SS and are capable of secreting the molecule of interest from the bacterial cytoplasm. In some embodiments, the secreted molecule, such as a heterologous protein or peptide comprises a type III secretion sequence that allows the molecule of interest to be secreted from the bacteria.

In some embodiments, a flagellar type III secretion pathway is used to secrete the molecule of interest. In some embodiments, an incomplete flagellum is used to secrete a therapeutic peptide of interest by recombinantly fusing the peptide to an N-terminal flagellar secretion signal of a native flagellar component. In this manner, the intracellularly expressed chimeric peptide can be mobilized across the inner and outer membranes into the surrounding host environment. For example, a modified flagellar type III secretion apparatus in which untranslated DNA fragment upstream of the gene fliC (encoding flagellin), e.g., a 173-bp region, is fused to the gene encoding the polypeptide of interest can be used to secrete heterologous polypeptides (See, e.g., Majander et al., Extracellular secretion of polypeptides using a modified Escherichia coli flagellar secretion apparatus. Nat Biotechnol. 2005 April; 23(4):475-81). In some cases, the untranslated region from the fliC loci, may not be sufficient to mediate translocation of the passenger peptide through the flagella. Here it may be necessary to extend the N-terminal signal into the amino acid coding sequence of FliC, for example using the 173 bp of untranslated region along with the first 20 amino acids of FliC (see, e.g., Duan et al., Secretion of Insulinotropic Proteins by Commensal Bacteria: Rewiring the Gut To Treat Diabetes, Appl. Environ. Microbiol. December 2008 vol. 74 no. 23 7437-7438).

In some embodiments, a Type V Autotransporter Secretion System is used to secrete the molecule of interest, e.g., therapeutic peptide. Due to the simplicity of the machinery and capacity to handle relatively large protein fluxes, the Type V secretion system is attractive for the extracellular production of recombinant proteins. As shown in FIG. 73, a therapeutic peptide (star) can be fused to an N-terminal secretion signal, a linker, and the beta-domain of an autotransporter. The N-terminal, Sec-dependent signal sequence directs the protein to the SecA-YEG machinery which moves the protein across the inner membrane into the periplasm, followed by subsequent cleavage of the signal sequence. The Beta-domain is recruited to the Bam complex (Teta-barrel assembly machinery′) where the beta-domain is folded and inserted into the outer membrane as a beta-barrel structure. The therapeutic peptide is threaded through the hollow pore of the beta-barrel structure ahead of the linker sequence. Once exposed to the extracellular environment, the therapeutic peptide can be freed from the linker system by an autocatalytic cleavage (left side of Bam complex) or by targeting of a membrane-associated peptidase (black scissors; right side of Bam complex) to a complimentary protease cut site in the linker. Thus, in some embodiments, the secreted molecule, such as a heterologous protein or peptide comprises an N-terminal secretion signal, a linker, and beta-domain of an autotransporter so as to allow the molecule to be secreted from the bacteria.

In some embodiments, a Hemolysin-based Secretion System is used to secrete the molecule of interest, e.g., therapeutic peptide. Type I Secretion systems offer the advantage of translocating their passenger peptide directly from the cytoplasm to the extracellular space, obviating the two-step process of other secretion types. FIG. 74 shows the alpha-hemolysin (HlyA) of uropathogenic Escherichia coli. This pathway uses HlyB, an ATP-binding cassette transporter; HlyD, a membrane fusion protein; and TolC, an outer membrane protein. The assembly of these three proteins forms a channel through both the inner and outer membranes. Natively, this channel is used to secrete HlyA, however, to secrete the therapeutic peptide of the present disclosure, the secretion signal-containing C-terminal portion of HlyA is fused to the C-terminal portion of a therapeutic peptide (star) to mediate secretion of this peptide.

In alternate embodiments, the genetically engineered bacteria further comprise a non-native single membrane-spanning secretion system. Single membrane-spanning transporters may act as a component of a secretion system, or may export substrates independently. Such transporters include, but are not limited to, ATP-binding cassette translocases, flagellum/virulence-related translocases, conjugation-related translocases, the general secretory system (e.g., the SecYEG complex in E. coli), the accessory secretory system in mycobacteria and several types of Gram-positive bacteria (e.g., Bacillus anthracis, Lactobacillus johnsonii, Corynebacterium glutamicum, Streptococcus gordonii, Staphylococcus aureus), and the twin-arginine translocation (TAT) system (Saier, 2006; Rigel and Braunstein, 2008; Albiniak et al., 2013). It is known that the general secretory and TAT systems can both export substrates with cleavable N-terminal signal peptides into the periplasm, and have been explored in the context of biopharmaceutical production. The TAT system may offer particular advantages, however, in that it is able to transport folded substrates, thus eliminating the potential for premature or incorrect folding. In certain embodiments, the genetically engineered bacteria comprise a TAT or a TAT-like system and are capable of secreting the molecule of interest from the bacterial cytoplasm. One of ordinary skill in the art would appreciate that the secretion systems disclosed herein may be modified to act in different species, strains, and subtypes of bacteria, and/or adapted to deliver different payloads.

In order to translocate a protein, e.g., therapeutic polypeptide, to the extracellular space, the polypeptide must first be translated intracellularly, mobilized across the inner membrane and finally mobilized across the outer membrane. Many effector proteins (e.g., therapeutic polypeptides)—particularly those of eukaryotic origin—contain disulphide bonds to stabilize the tertiary and quaternary structures. While these bonds are capable of correctly forming in the oxidizing periplasmic compartment with the help of periplasmic chaperones, in order to translocate the polypeptide across the outer membrane the disulphide bonds must be reduced and the protein unfolded again.

One way to secrete properly folded proteins in gram-negative bacteria—particularly those requiring disulphide bonds—is to target the reducing-environment periplasm in conjunction with a destabilizing outer membrane. In this manner, the protein is mobilized into the oxidizing environment and allowed to fold properly. In contrast to orchestrated extracellular secretion systems, the protein is then able to escape the periplasmic space in a correctly folded form by membrane leakage. These “leaky” gram-negative mutants are therefore capable of secreting bioactive, properly disulphide-bonded polypeptides. In some embodiments, the genetically engineered bacteria have a “leaky” or de-stabilized outer membrane. Destabilizing the bacterial outer membrane to induce leakiness can be accomplished by deleting or mutagenizing genes responsible for tethering the outer membrane to the rigid peptidoglycan skeleton, including for example, lpp, ompC, ompA, ompF, tolA, tolB, pal, degS, degP, and nlpI. Lpp is the most abundant polypeptide in the bacterial cell existing at ˜500,000 copies per cell and functions as the primary ‘staple’ of the bacterial cell wall to the peptidoglycan. 1. Silhavy, T. J., Kahne, D. & Walker, S. The bacterial cell envelope. Cold Spring Harb Perspect Biol 2, a000414 (2010). TolA-PAL and OmpA complexes function similarly to Lpp and are other deletion targets to generate a leaky phenotype. Additionally, leaky phenotypes have been observed when periplasmic proteases are inactivated. The periplasm is very densely packed with protein and therefore encode several periplasmic proteins to facilitate protein turnover. Removal of periplasmic proteases such as degS, degP or nlpI can induce leaky phenotypes by promoting an excessive build-up of periplasmic protein. Mutation of the proteases can also preserve the effector polypeptide by preventing targeted degradation by these proteases. Moreover, a combination of these mutations may synergistically enhance the leaky phenotype of the cell without major sacrifices in cell viability. Thus, in some embodiments, the engineered bacteria have one or more deleted or mutated membrane genes. In some embodiments, the engineered bacteria have a deleted or mutated lpp gene. In some embodiments, the engineered bacteria have one or more deleted or mutated gene(s), selected from ompA, ompA, and ompF genes. In some embodiments, the engineered bacteria have one or more deleted or mutated gene(s), selected from tolA, tolB, and pal genes. in some embodiments, the engineered bacteria have one or more deleted or mutated periplasmic protease genes. In some embodiments, the engineered bacteria have one or more deleted or mutated periplasmic protease genes selected from degS, degP, and nlpI. In some embodiments, the engineered bacteria have one or more deleted or mutated gene(s), selected from lpp, ompA, ompF, tolA, tolB, pal, degS, degP, and nlpI genes.

To minimize disturbances to cell viability, the leaky phenotype can be made inducible by placing one or more membrane or periplasmic protease genes, e.g., selected from lpp, ompA, ompF, tolA, tolB, pal, degS, degP, and nlpI, under the control of an inducible promoter. For example, expression of lpp or other cell wall stability protein or periplasmic protease can be repressed in conditions where the therapeutic polypeptide needs to be delivered (secreted). For instance, under inducing conditions a transcriptional repressor protein or a designed antisense RNA can be expressed which reduces transcription or translation of a target membrane or periplasmic protease gene. Conversely, overexpression of certain peptides can result in a destabilized phenotype, e.g., over expression of colicins or the third topological domain of TolA, which peptide overexpression can be induced in conditions in which the therapeutic polypeptide needs to be delivered (secreted). These sorts of strategies would decouple the fragile, leaky phenotypes from biomass production. Thus, in some embodiments, the engineered bacteria have one or more membrane and/or periplasmic protease genes under the control of an inducible promoter.

Table 11 and Table 12A list secretion systems for Gram positive bacteria and Gram negative bacteria. These can be used to secrete polypeptides, proteins of interest or therapeutic protein(s) from the engineered bacteria, which are reviewed in Milton H. Saier, Jr. Microbe/Volume 1, Number 9, 2006 “Protein Secretion Systems in Gram-Negative Bacteria Gram-negative bacteria possess many protein secretion-membrane insertion systems that apparently evolved independently”, the contents of which is herein incorporated by reference in its entirety.

TABLE 11
Secretion systems for gram positive bacteria

| Bacterial Strain | Relevant Secretion System |
|---|---|
| C. novyi-NT (Gram+) | Sec pathway |
| | Twin- arginine (TAT) pathway |
| C. butryicum (Gram+) | Sec pathway |
| | Twin- arginine (TAT) pathway |
| Listeria monocytogenes (Gram+) | Sec pathway |
| | Twin- arginine (TAT) pathway |

TABLE 12A
Secretion Systems for Gram negative bacteria
Protein secretary pathways (SP) in gram-negative bacteria and their descendants

| | | | | | | | |
|---|---|---|---|---|---|---|---|
| | | | | | | # | |
| Type | | | | | | Proteins/ | Energy |
| (Abbreviation) | Name | TC#² | Bacteria | Archaea | Eukarya | System | Source |

IMPS - Gram-negative bacterial inner membrane channel-forming translocases

| | | | | | | | |
|---|---|---|---|---|---|---|---|
| ABC | ATP binding | 3.A.1 | + | + | + | 3-4 | ATP |
| (SIP) | cassette | | | | | | |
| | translocase | | | | | | |
| SEC | General | 3.A.5 | + | + | + | ~12 | GTP OR |
| (IISP) | secretory | | | | | | ATP + |
| | translocase | | | | | | PMF |
| Fla/Path | Flagellum/ | 3.A.6 | + | − | − | >10 | ATP |
| (IIISP) | virulence- | | | | | | |
| | related | | | | | | |
| | translocase | | | | | | |
| Conj | Conjugation- | 3.A.7 | + | − | − | >10 | ATP |
| (IVSP) | related | | | | | | |
| | translocase | | | | | | |
| Tat | Twin- | 2.A.6 | + | + | + | 2-4 | PMF |
| (IISP) | arginine | 4 | | | (chloroplasts) | | |
| | targeting | | | | | | |
| | translocase | | | | | | |
| Oxa1 | Cytochrome | 2.A.9 | + | + | + | 1 | None or |
| (YidC) | oxidase | | | | (mitochondria | | PMF |
| | biogenesis | | | | chloroplasts) | | |
| | family | | | | | | |
| MscL | Large | 1.A.2 | + | + | + | 1 | None |
| | conductance | 2 | | | | | |
| | mechanosensitive | | | | | | |
| | channel family | | | | | | |
| Holins | Holin | 1.E.1 | + | − | − | 1 | None |
| | functional | •21 | | | | | |
| | superfamily | | | | | | |

Eukaryotic Organelles

| | | | | | | | |
|---|---|---|---|---|---|---|---|
| MPT | Mitochondrial | 3.A.B | − | − | + | >20 | ATP |
| | protein | | | | (mitochondrial) | | |
| | translocase | | | | | | |
| CEPT | Chloroplast | 3.A.9 | (+) | − | + | ≧3 | GTP |
| | envelope | | | | (chloroplasts) | | |
| | protein | | | | | | |
| | translocase | | | | | | |
| Bcl-2 | Eukaryotic | 1.A.2 | − | − | + | 1? | None |
| | Bcl-2 family | 1 | | | | | |
| | (programmed | | | | | | |
| | cell death) | | | | | | |

Gram-negative bacterial outer membrane channel-forming translocases

| | | | | | | | |
|---|---|---|---|---|---|---|---|
| MTB | Main | 3.A.1 | +^{b} | − | − | ~14 | ATP; |
| (IISP) | terminal | 5 | | | | | PMF |
| | branch of | | | | | | |
| | the general | | | | | | |
| | secretory | | | | | | |
| | translocase | | | | | | |
| FUP AT-1 | Fimbrial | 1.B.1 | +^{b} | − | − | 1 | None |
| | usher protein | 1 | +^{b} | | − | 1 | None |
| | Auto- | l.B.l | | | | | |
| | transporter-1 | 2 | | | | | |
| AT-2 | Auto- | 1.B.4 | +^{b} | − | − | 1 | None |
| OMF | transporter-2 | 0 | +^{b} | | +(?) | 1 | None |
| (ISP) | | 1.B.1 | | | | | |
| | | 7 | | | | | |
| TPS | | 1.B.2 | + | − | + | 1 | None |
| Secretin | | 0 | +^{b} | | − | 1 | None |
| (IISP and | | 1.B.2 | | | | | |
| IISP) | | 2 | | | | | |
| OmpIP | Outer | 1.B.3 | + | − | + | ≧4 | None |
| | membrane | 3 | | | (mitochondria; | | ? |
| | insertion | | | | chloroplasts) | | |
| | porin | | | | | | |

The above tables for gram positive and gram negative bacteria list secretion systems that can be used to secrete polypeptides and other molecules from the engineered bacteria, which are reviewed in Milton H. Saier, Jr. Microbe/Volume 1, Number 9, 2006 “Protein Secretion Systems in Gram-Negative Bacteria Gram-negative bacteria possess many protein secretion-membrane insertion systems that apparently evolved independently”, the contents of which is herein incorporated by reference in its entirety.

TABLE 12B
Comparison of Secretion systems for secretion
of polypeptide from engineered bacteria

| Secretion | | | | |
|---|---|---|---|---|
| System | Tag | Cleavage | Advantages | Other features |
| Modified | mRNA | No | No peptide | May not be as suited |
| Type III | (or N- | cleavage | tag | for larger proteins |
| (flagellar) | terminal) | necessary | Endogenous | Deletion of flagellar |
| | | | | genes |
| Type V | N- and | Yes | Large | 2-step secretion |
| autotransport | C-terminal | | proteins | |
| | | | Endogenous | |
| | | | Cleavable | |
| Type I | C-terminal | No | | Tag; Exogenous |
| | | | | Machinery |
| Diffusible | N-terminal | Yes | Disulfide | May affect cell |
| Outer | | | bond | fragility/ |
| Membrane | | | formation | survivability/ |
| (DOM) | | | | growth/yield |

In some embodiments, one or more BCAA catabolic enzymes described herein are secreted. In some embodiments, the one or more BCAA catabolic enzymes described herein are further modified to improve secretion efficiency, decreased susceptibility to proteases, stability, and/or half-life. In some embodiments, leucine dehydrogenase is secreted, alone or in combination other BCAA catabolic enzymes, e.g., with a ketoacid decarboxylase and/or an alcohol dehydrogenase. In some embodiments, leucine dehydrogenase is secreted, alone or in combination other BCAA catabolic enzymes, e.g., with a ketoacid decarboxylase and/or an aldehyde dehydrogenase. In some embodiments, BCAA aminotransferase is secreted, alone or in combination other BCAA catabolic enzymes, e.g., with a ketoacid decarboxylase and/or an alcohol dehydrogenase dehydrogenase. In some embodiments, BCAA aminotransferase is secreted, alone or in combination other BCAA catabolic enzymes, e.g., with a ketoacid decarboxylase and/or an aldehyde dehydrogenase. In some embodiments, amino acid oxidase is secreted, alone or in combination other BCAA catabolic enzymes, e.g., with a ketoacid decarboxylase and/or an alcohol dehydrogenase dehydrogenase. In some embodiments, amino acid oxidase is secreted, alone or in combination other BCAA catabolic enzymes, e.g., with a ketoacid decarboxylase and/or an aldehyde dehydrogenase. In some embodiments, a ketoacid carboxylase is secreted, alone or in combination with other BCAA catabolic enzymes. In some embodiments, an alcohol dehydrogenase is secreted, alone or in combination with other BCAA catabolic enzymes. In some embodiments, an aldehyde dehydrogenase is secreted, alone or in combination with other BCAA catabolic enzymes.

In some embodiments, leucine dehydrogenase is secreted, alone or in combination other BCAA catabolic enzymes, e.g., with one or more Bkd complex enzymes, and/or one or more Liu operon enzymes. In some embodiments, leucine dehydrogenase is secreted, alone or in combination other BCAA catabolic enzymes, e.g., with one or more Bkd complex enzymes.

In some embodiments, BCAA aminotransferase is secreted, alone or in combination other BCAA catabolic enzymes, e.g., with one or more Bkd complex enzymes, and/or one or more Liu operon enzymes. In some embodiments, BCAA aminotransferase is secreted, alone or in combination other BCAA catabolic enzymes, e.g., with one or more Bkd complex enzymes. In some embodiments, amino acid oxidase is secreted, alone or in combination other BCAA catabolic enzymes, e.g., with one or more Bkd complex enzymes, and/or one or more Liu operon enzymes. In some embodiments, amino acid oxidase is secreted, alone or in combination other BCAA catabolic enzymes, e.g., with one or more Bkd complex enzymes.

In some embodiments, one or more enzymes from the Bkd complex are secreted, alone ore in combination with one or more BCAA catabolic enzymes. In some embodiments, one or more enzymes from the Bkd complex are secreted, alone ore in combination with one or more Liu operon enzymes. In some embodiments, Lbul is secreted, alone or in combination with one or more BCAA catabolic enzymes.

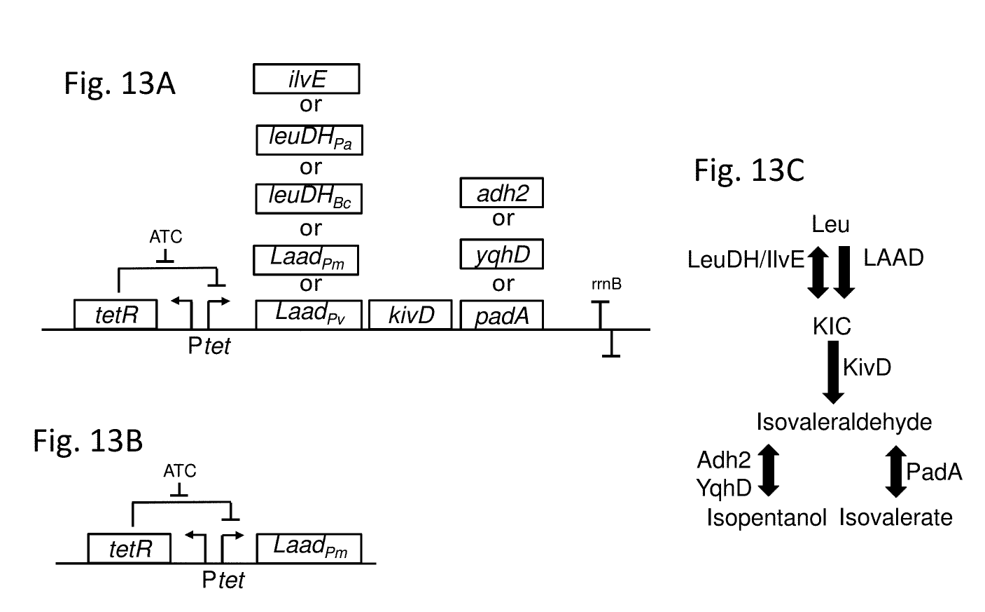

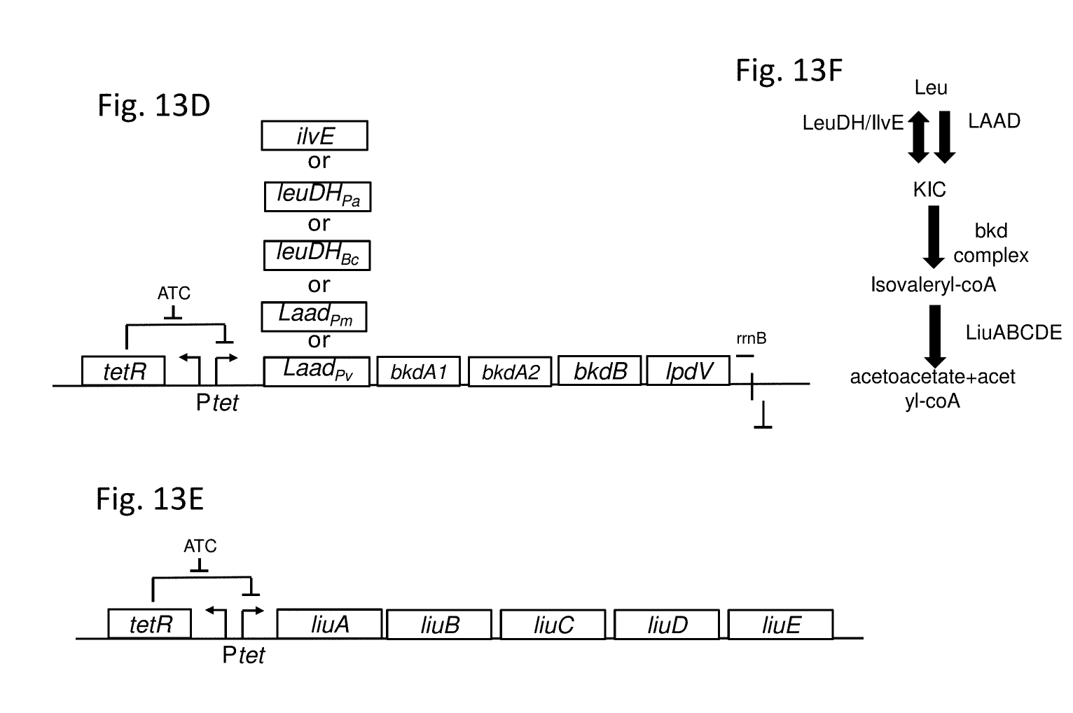

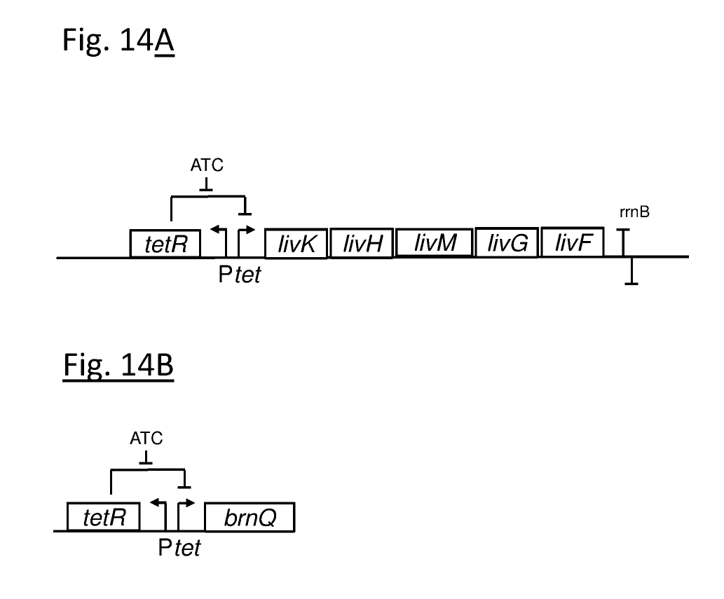

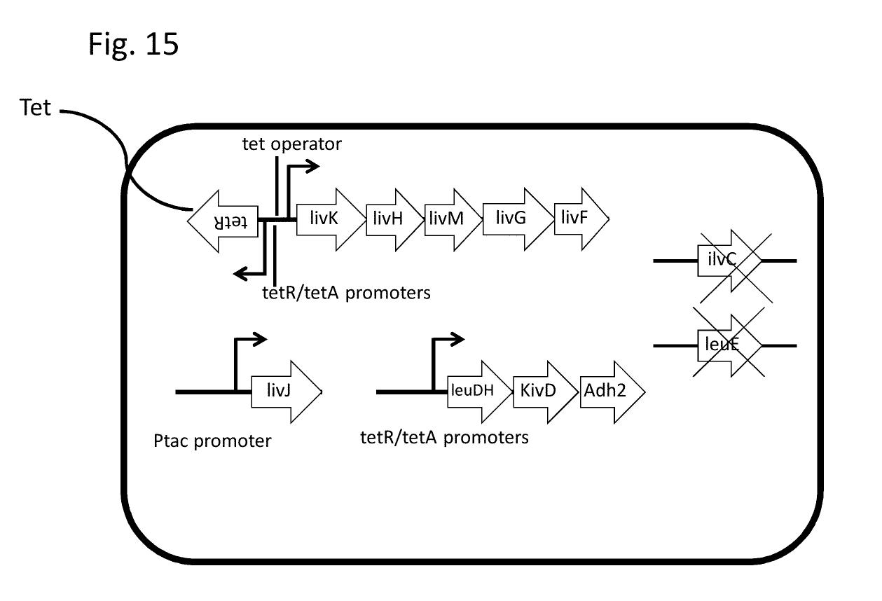

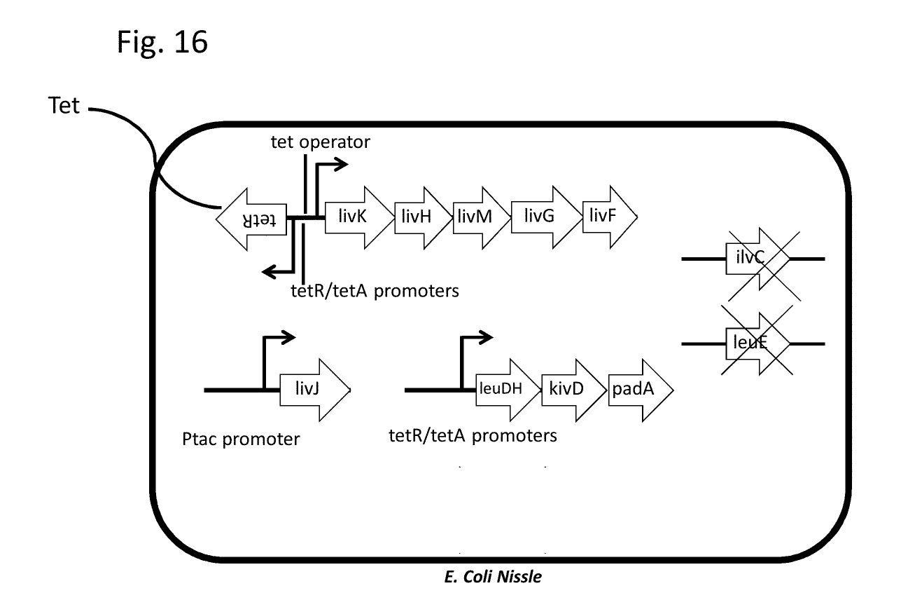

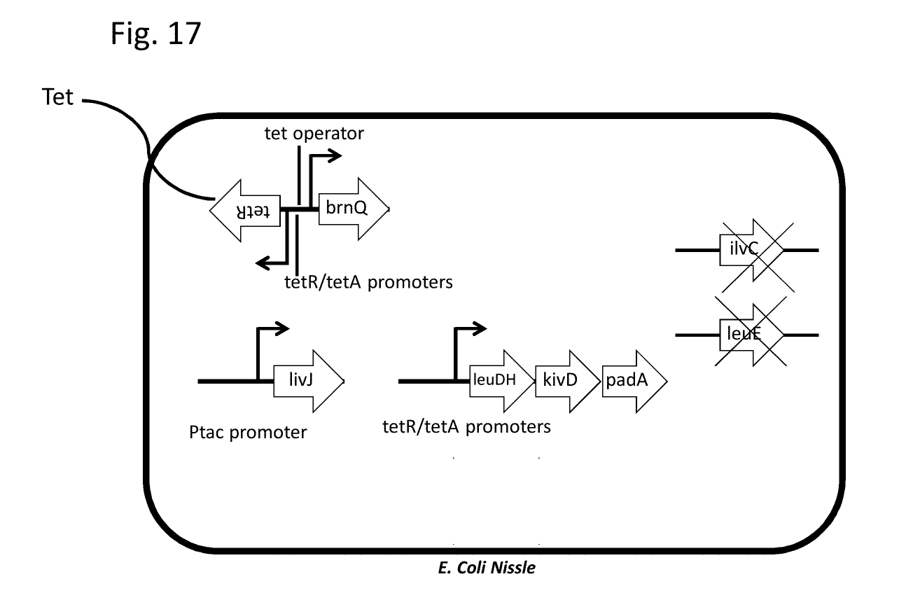

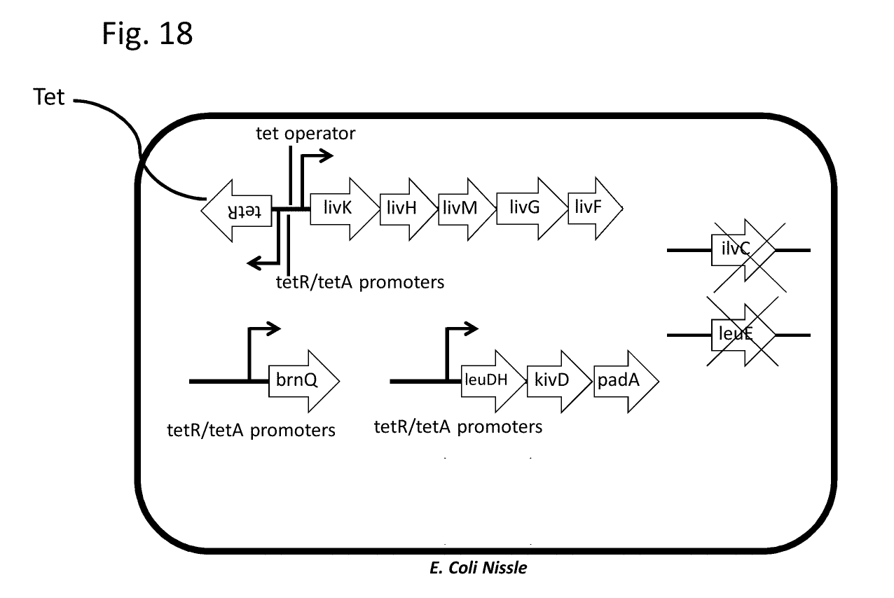

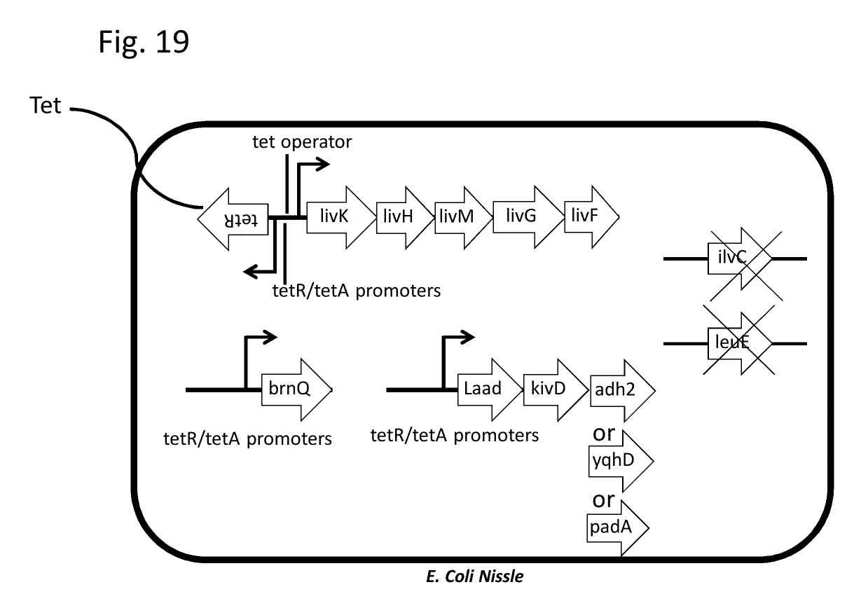

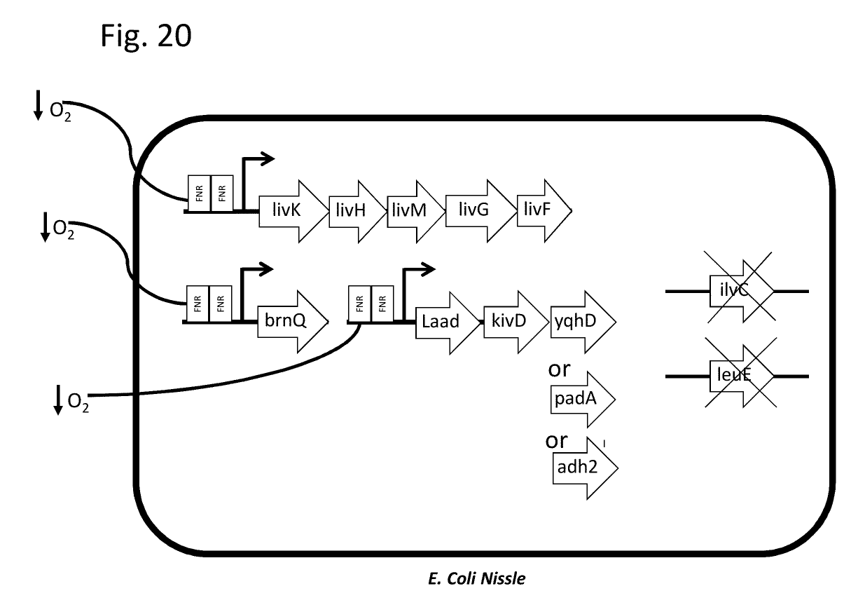

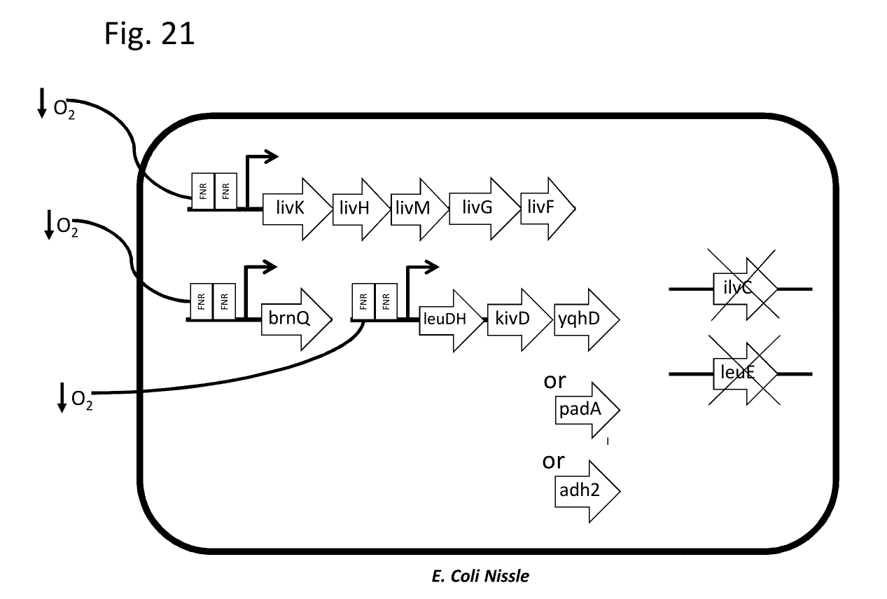

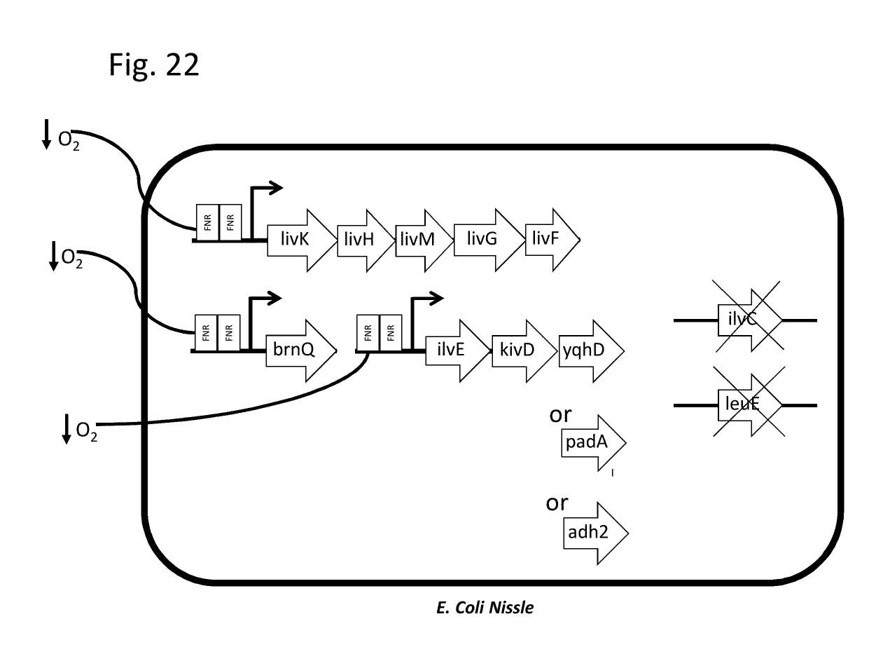

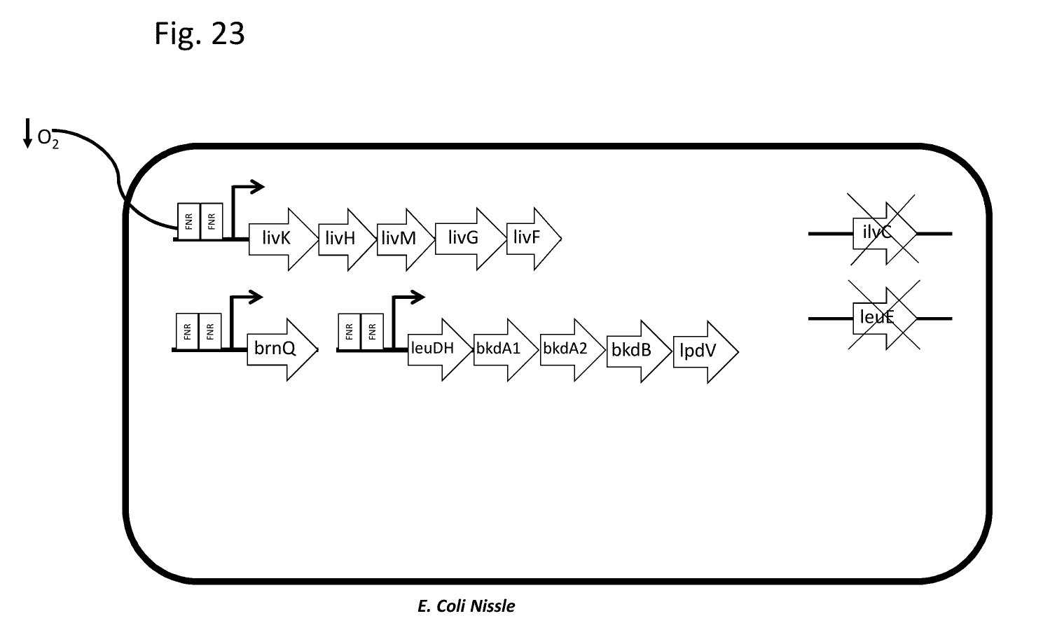

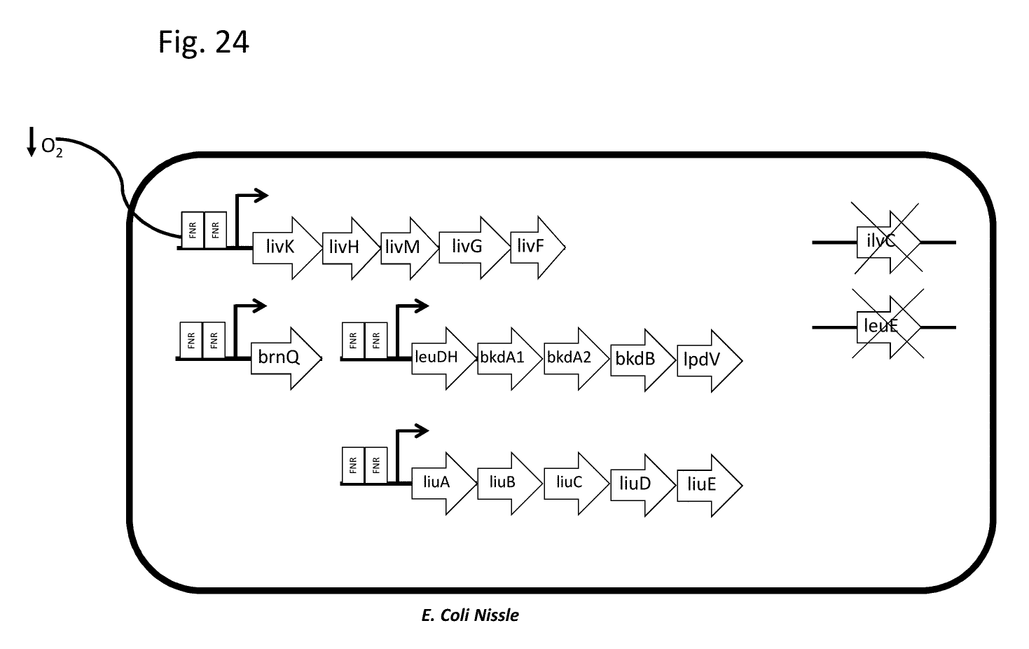

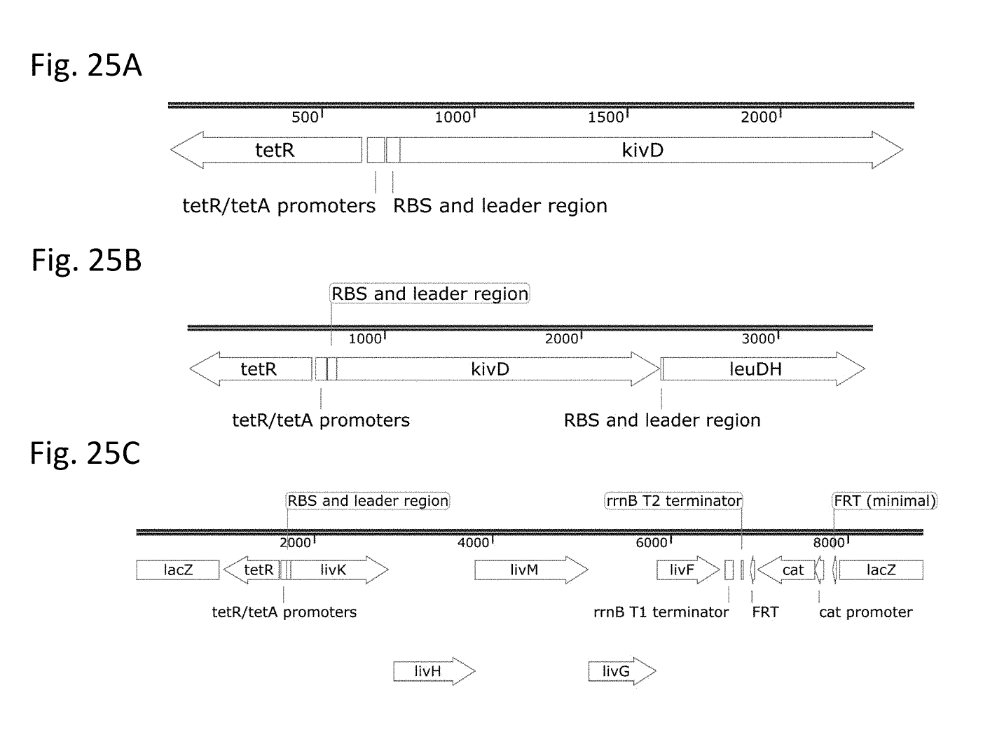

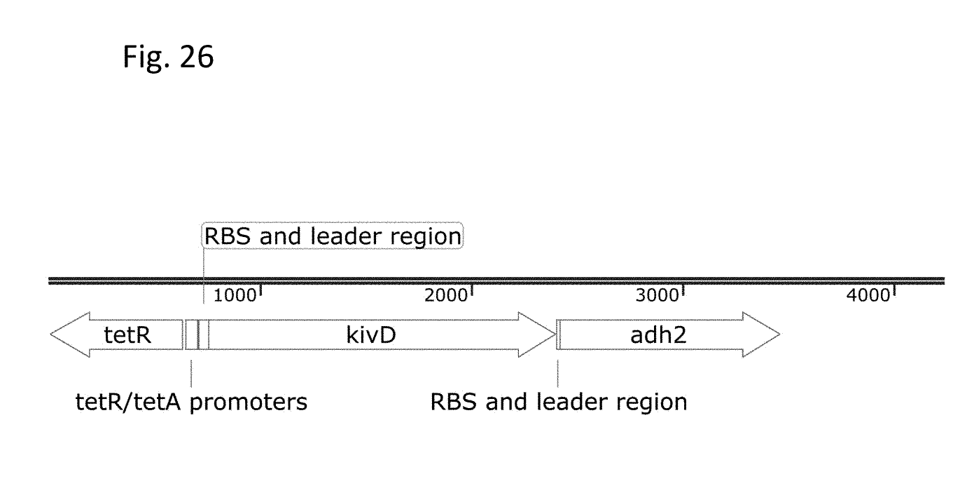

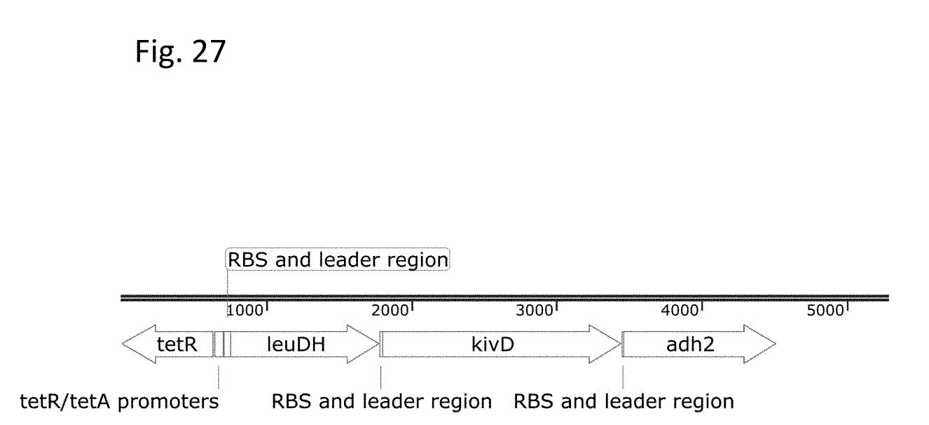

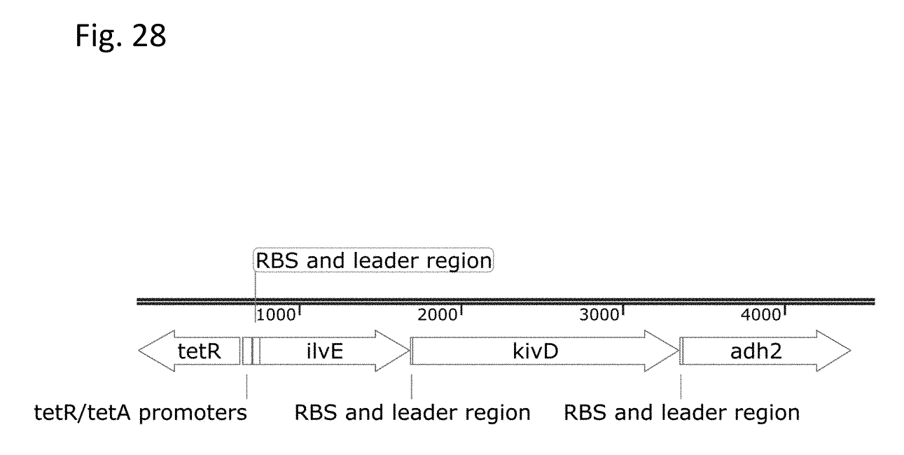

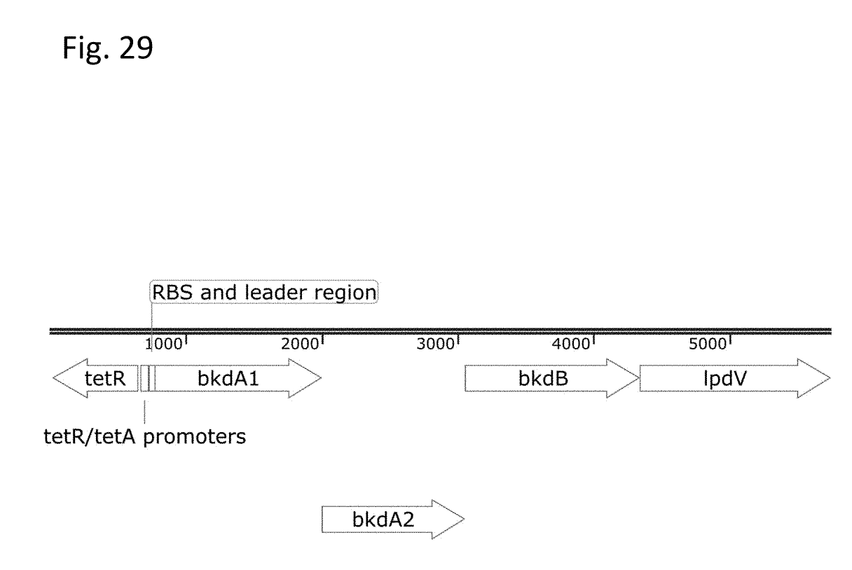

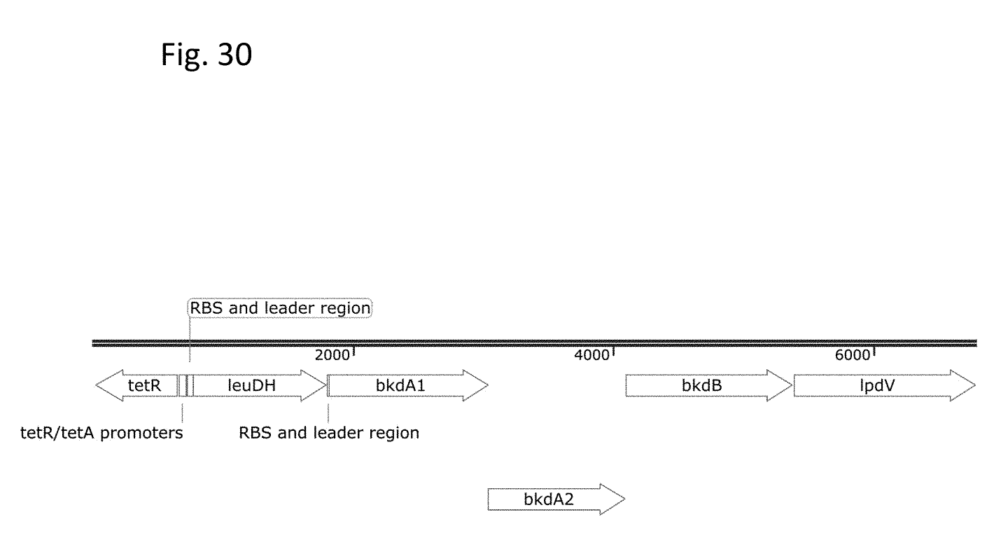

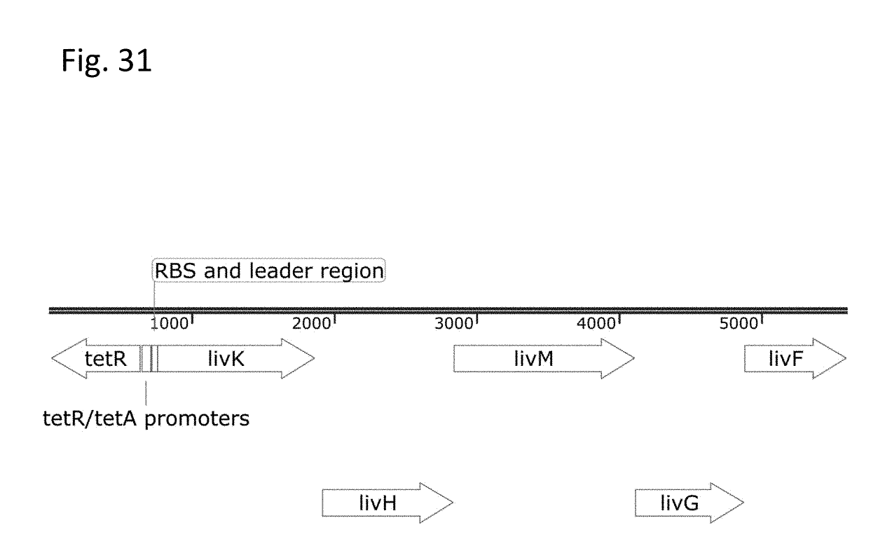

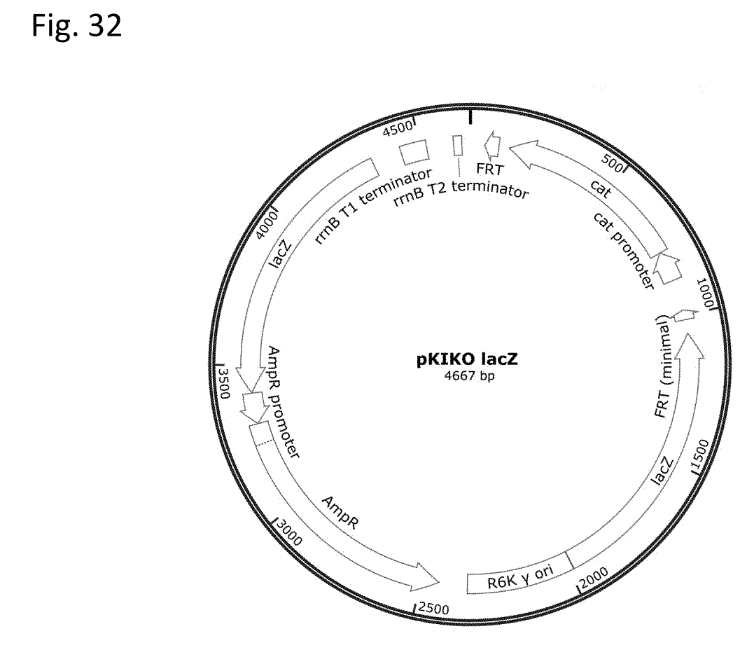

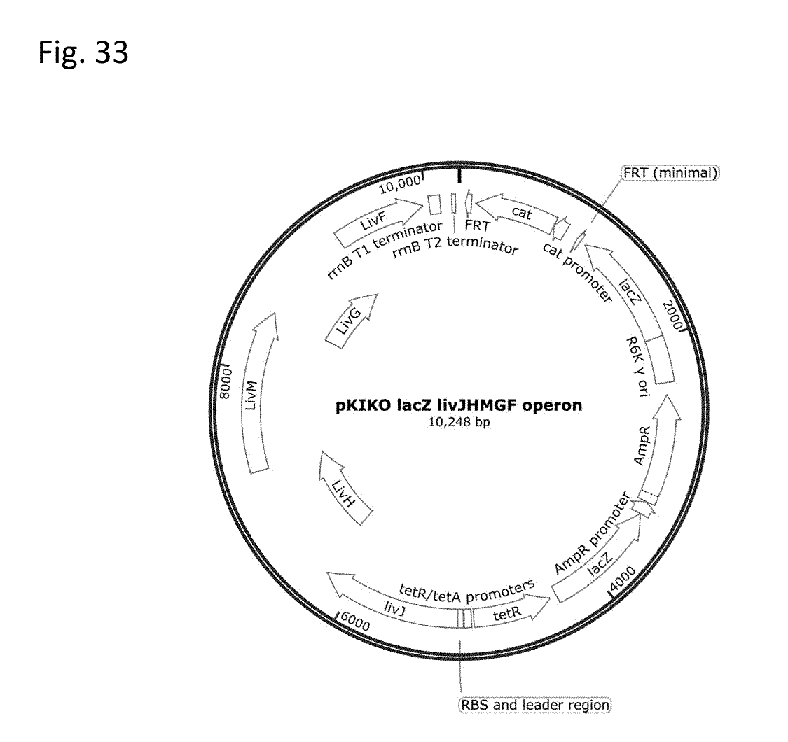

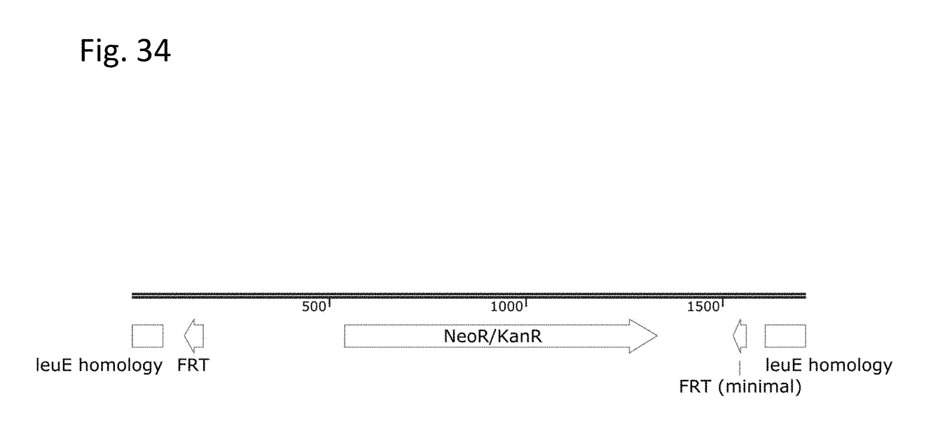

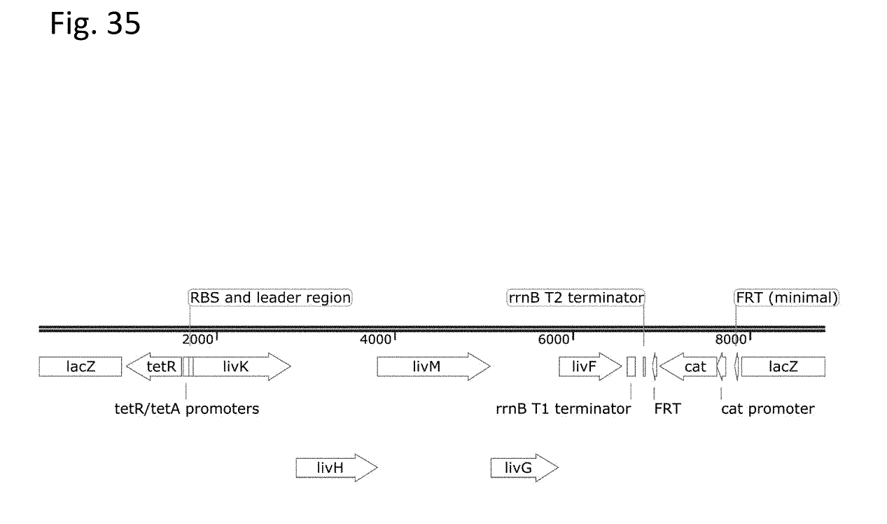

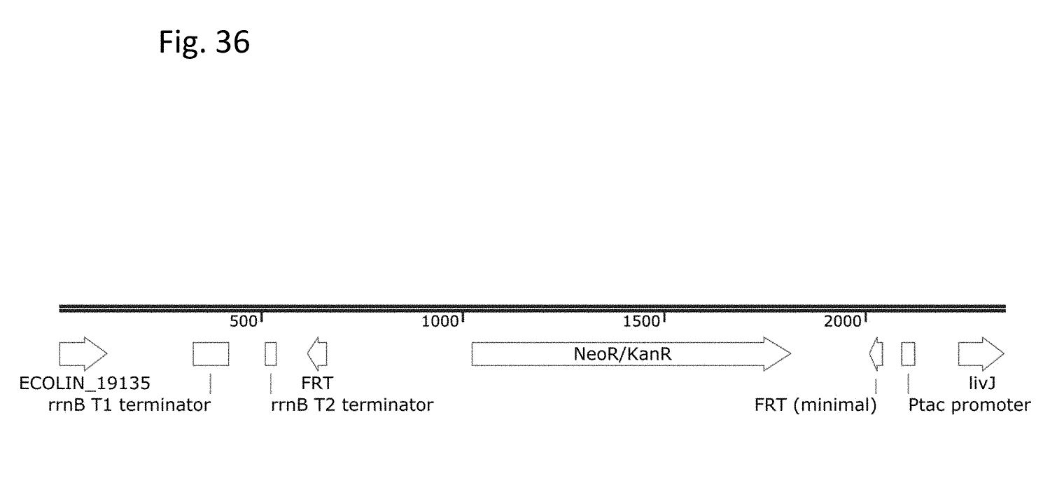

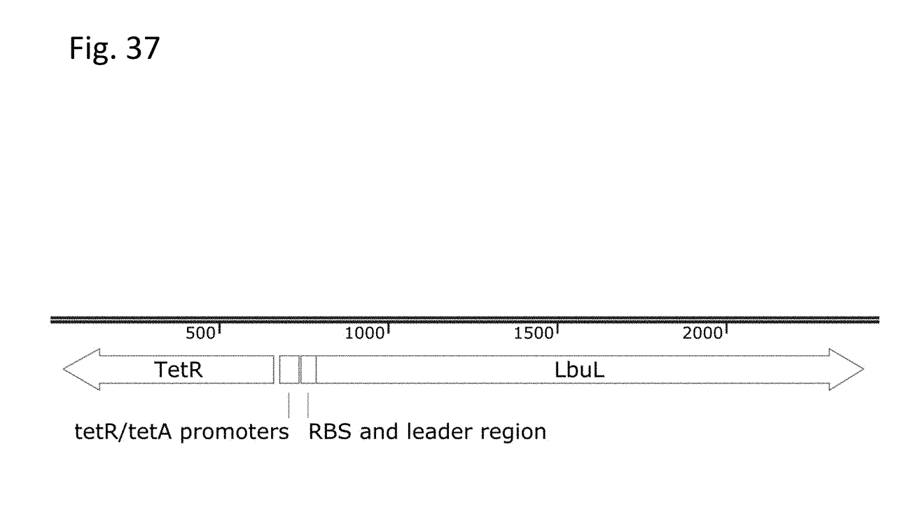

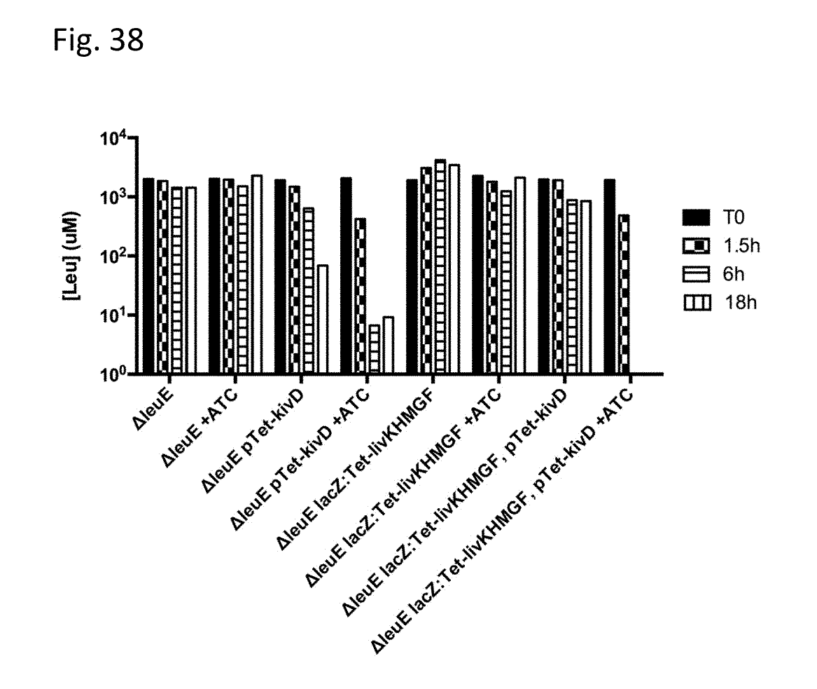

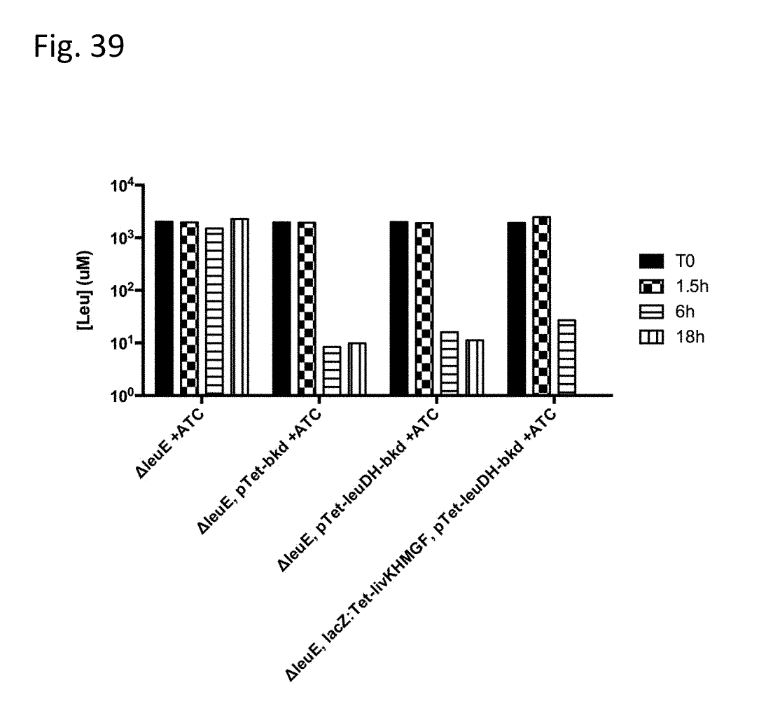

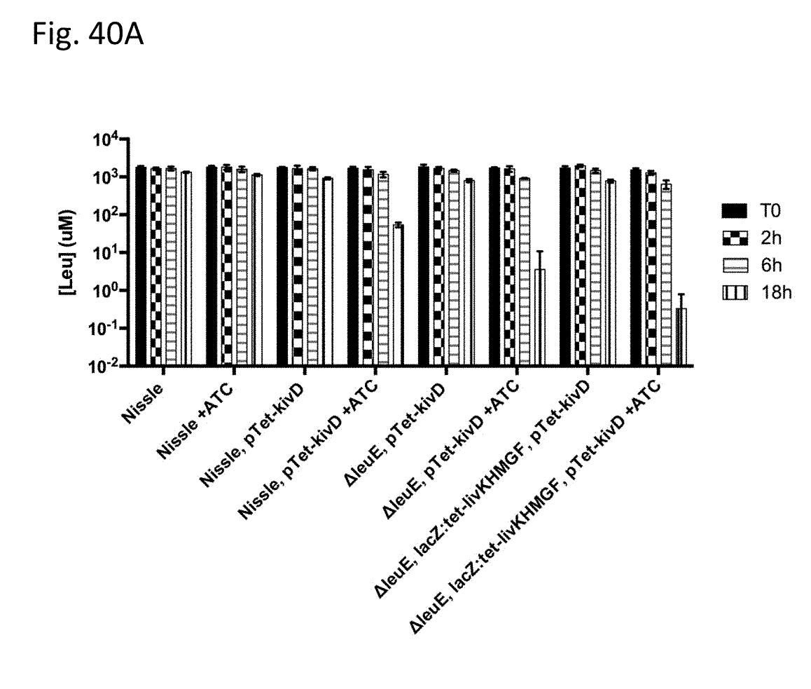

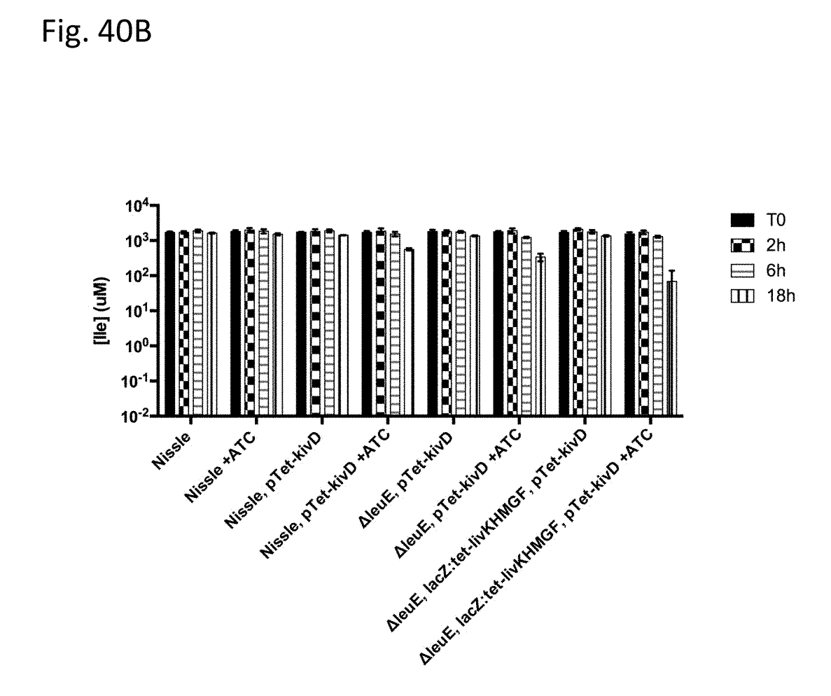

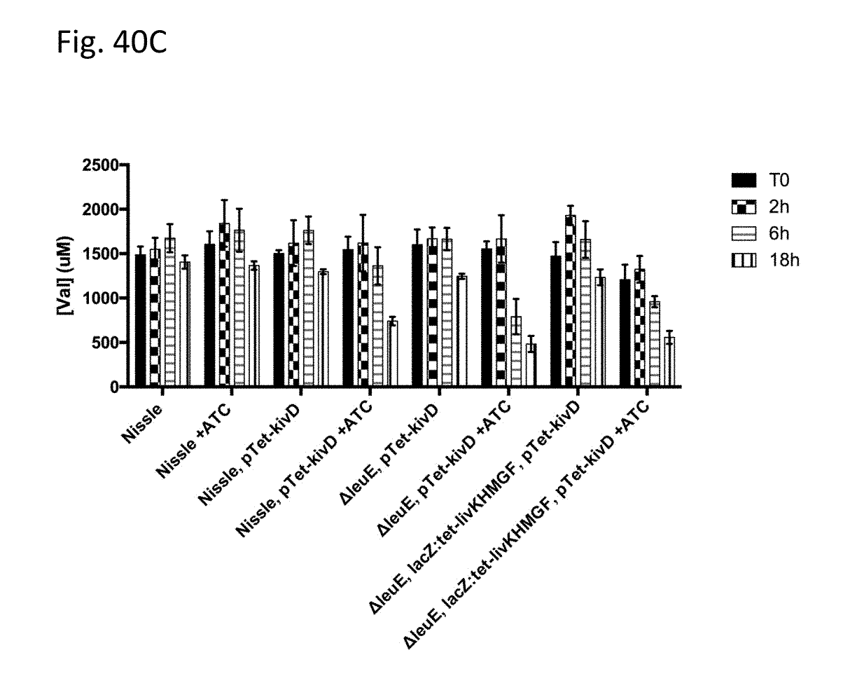

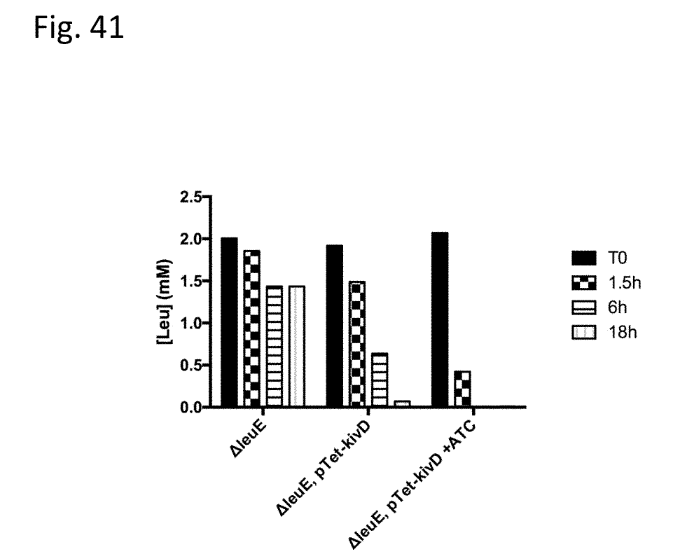

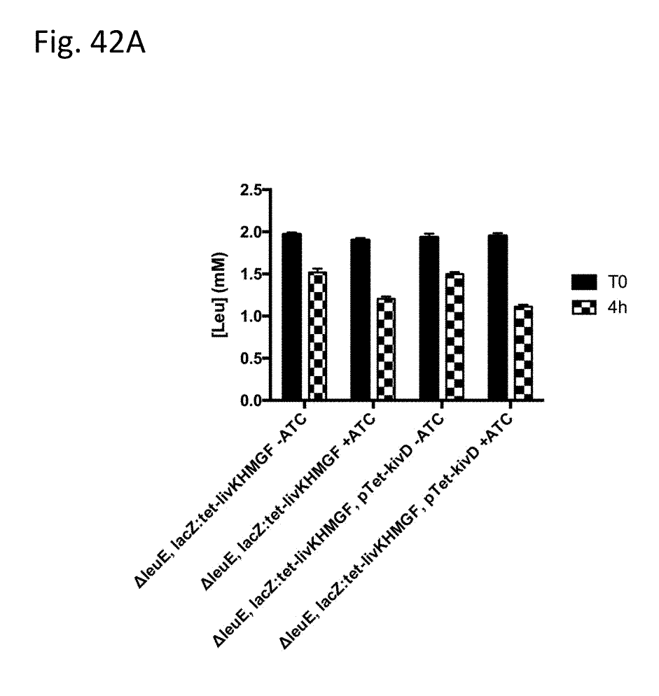

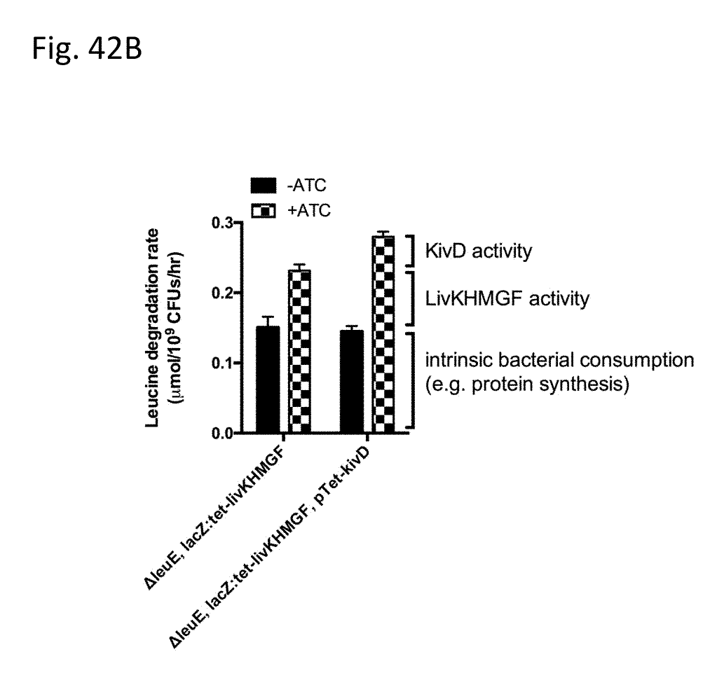

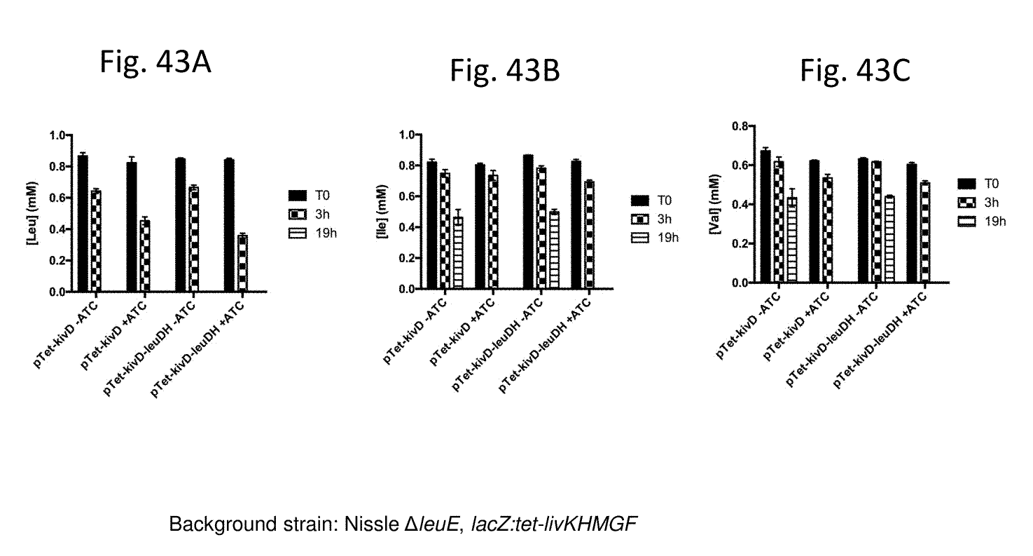

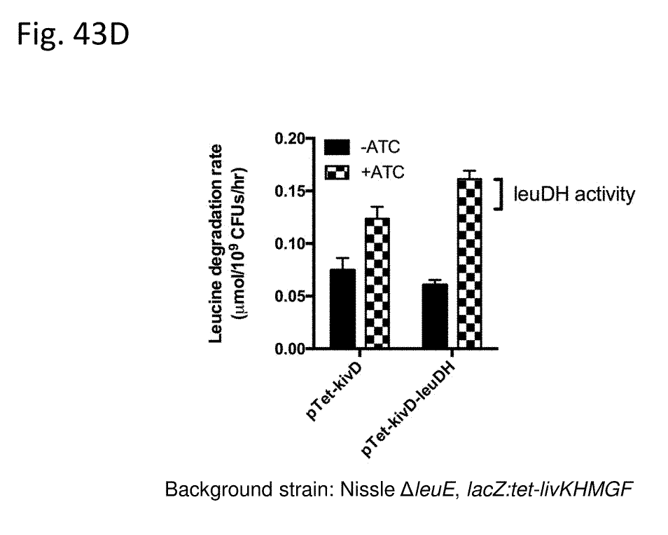

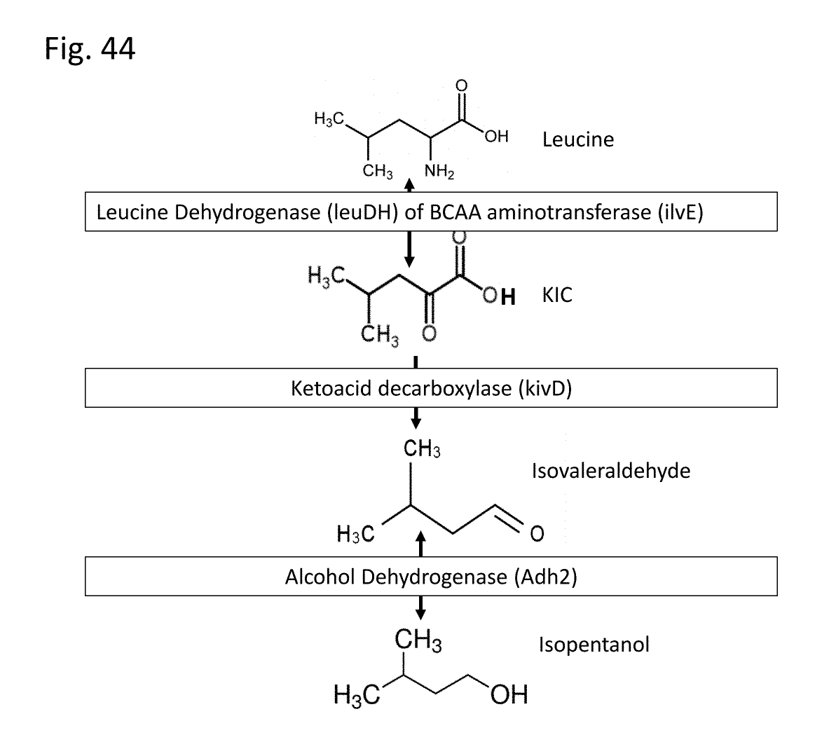

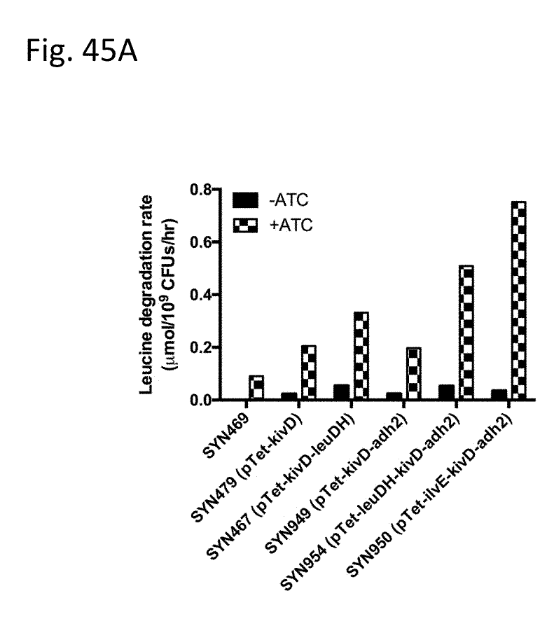

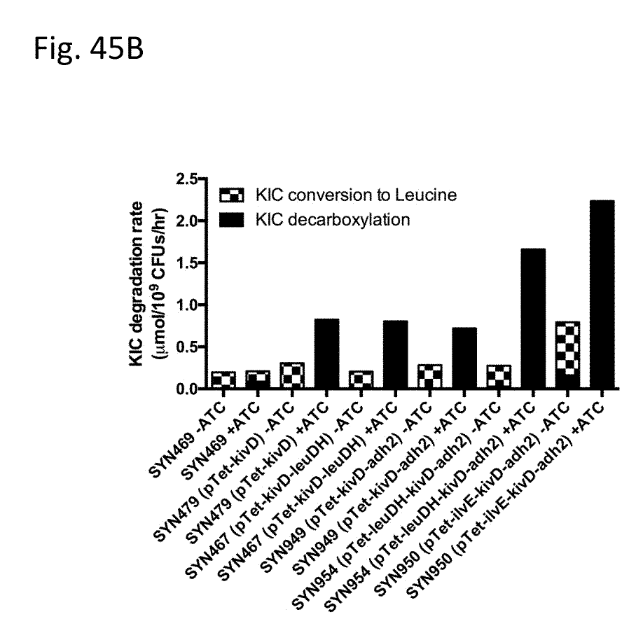

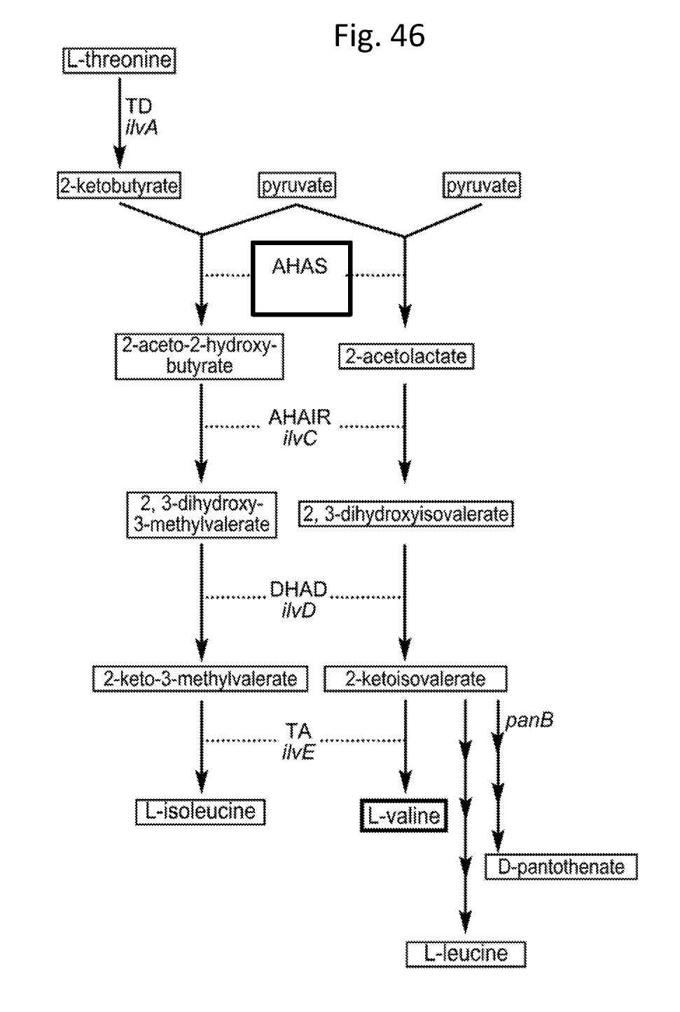

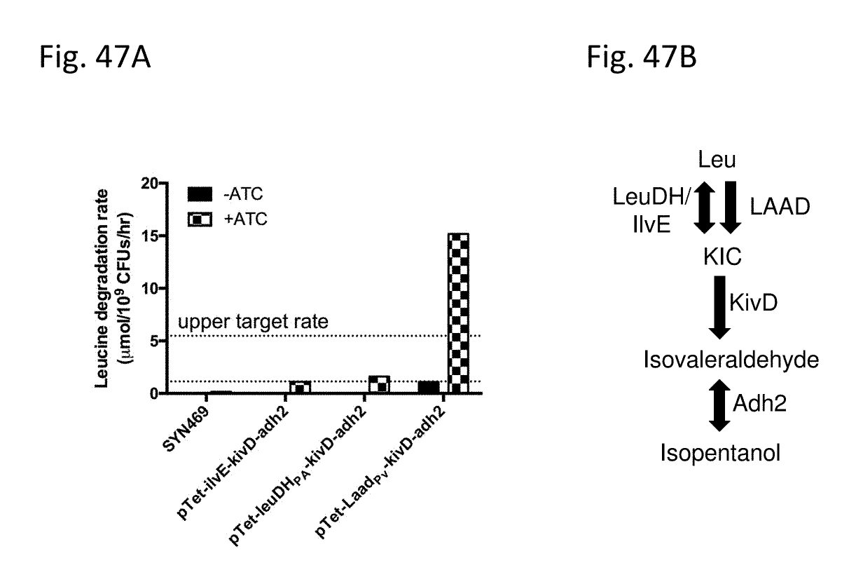

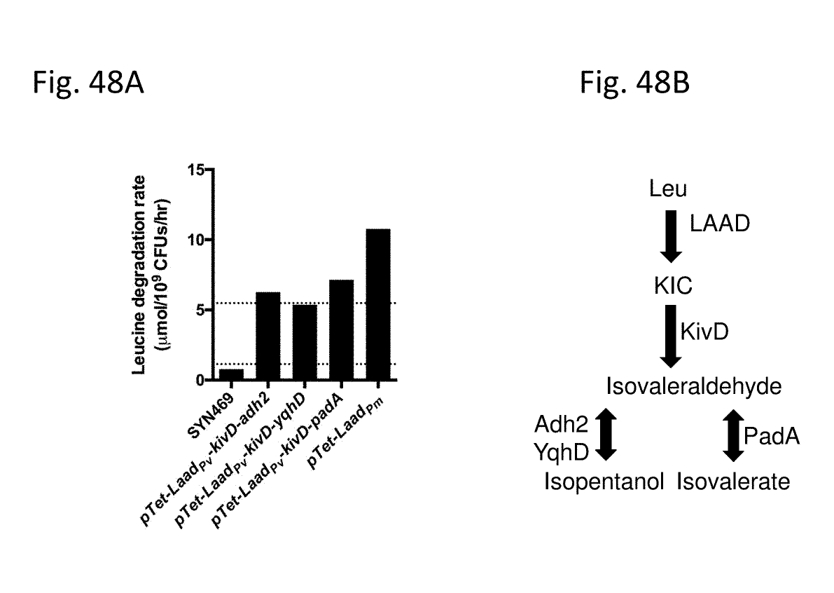

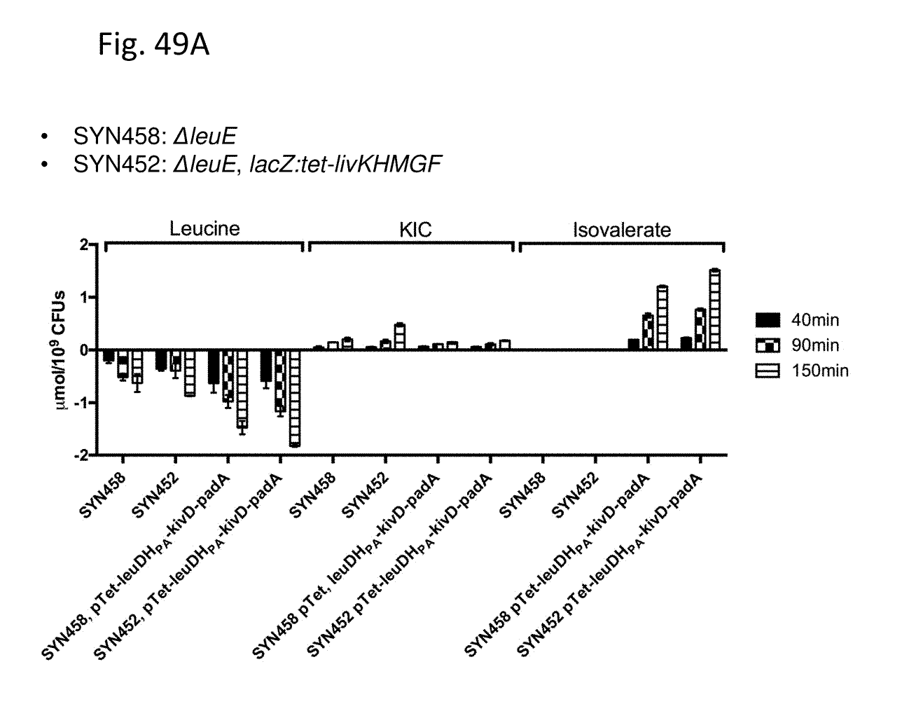

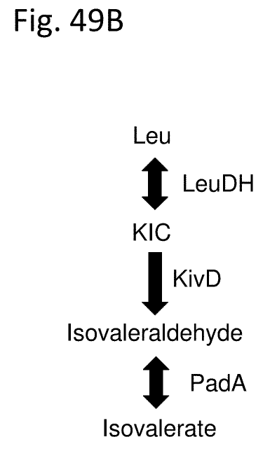

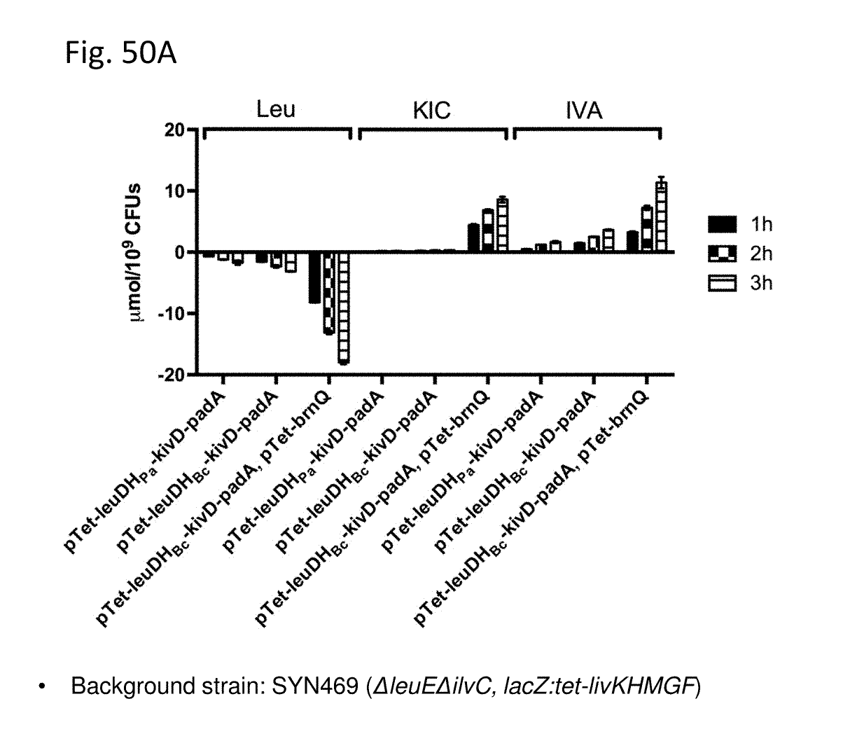

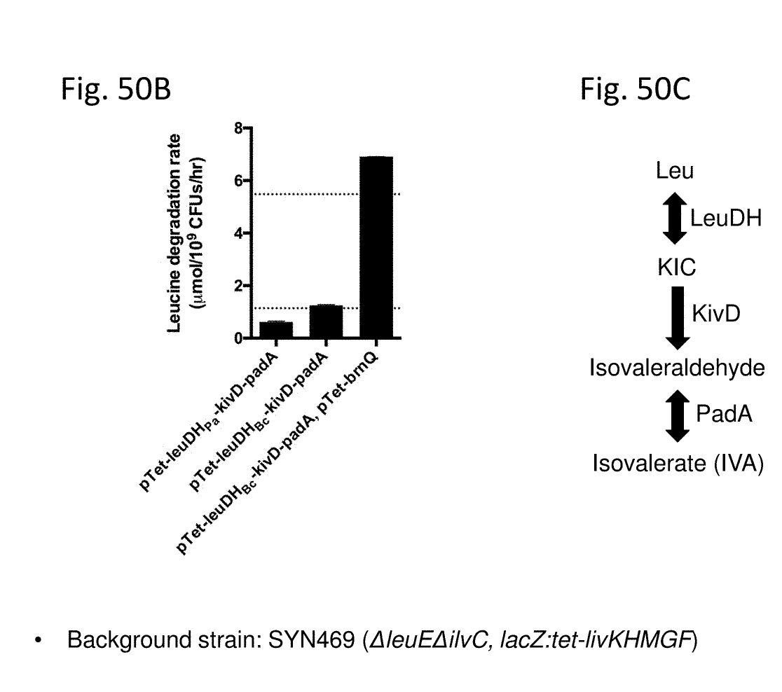

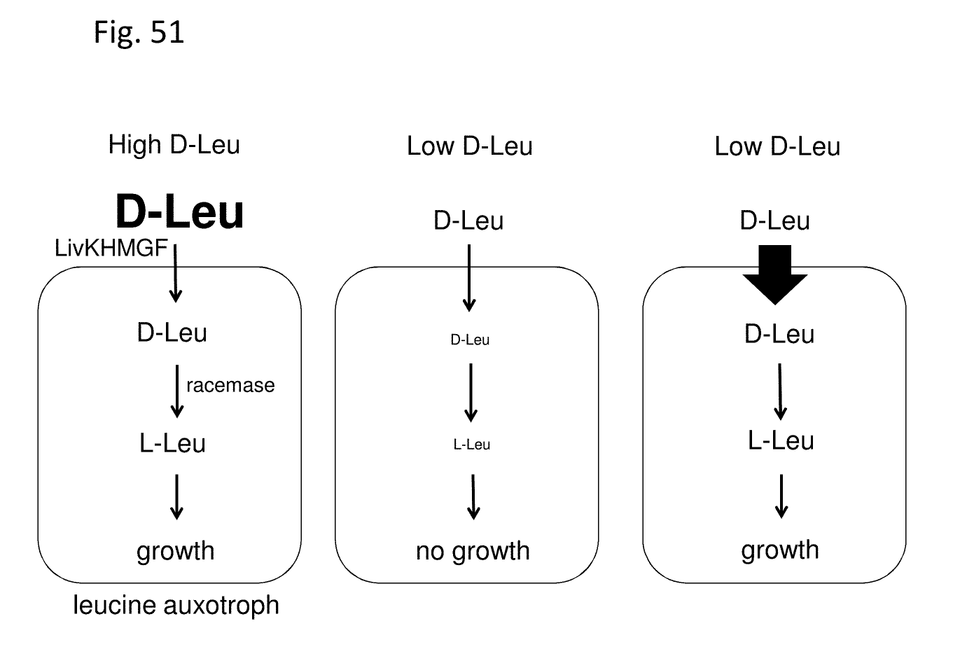

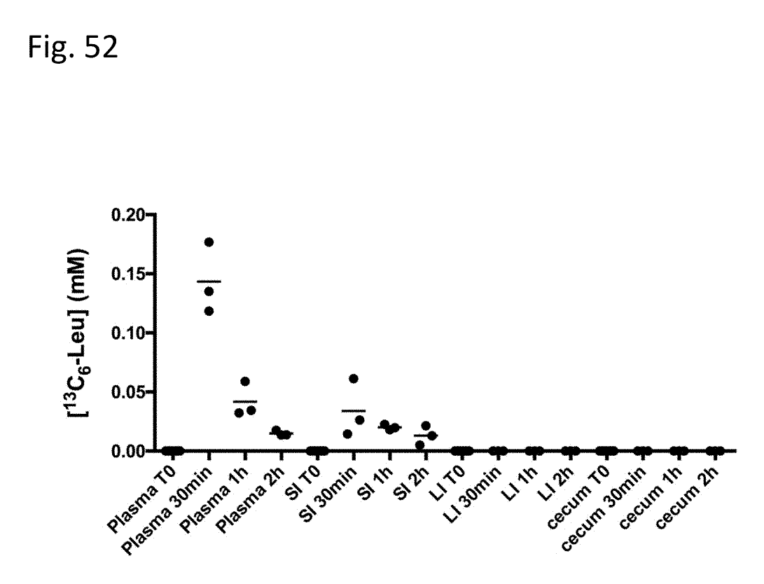

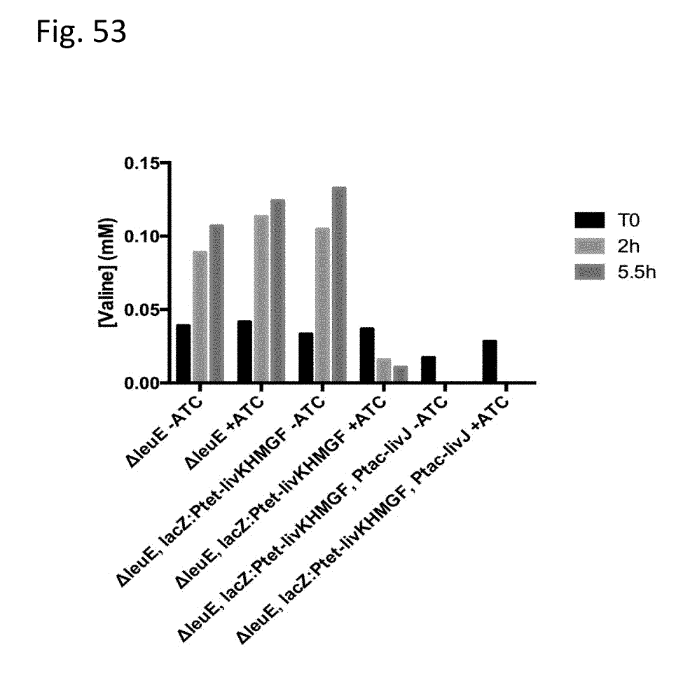

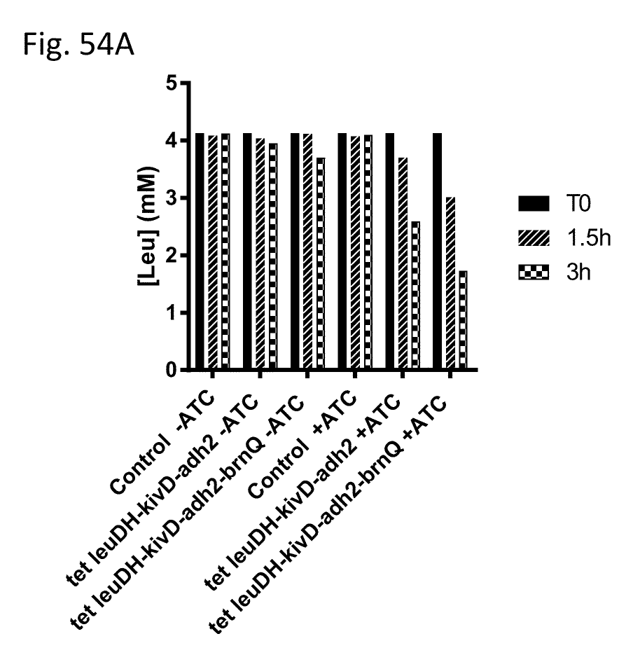

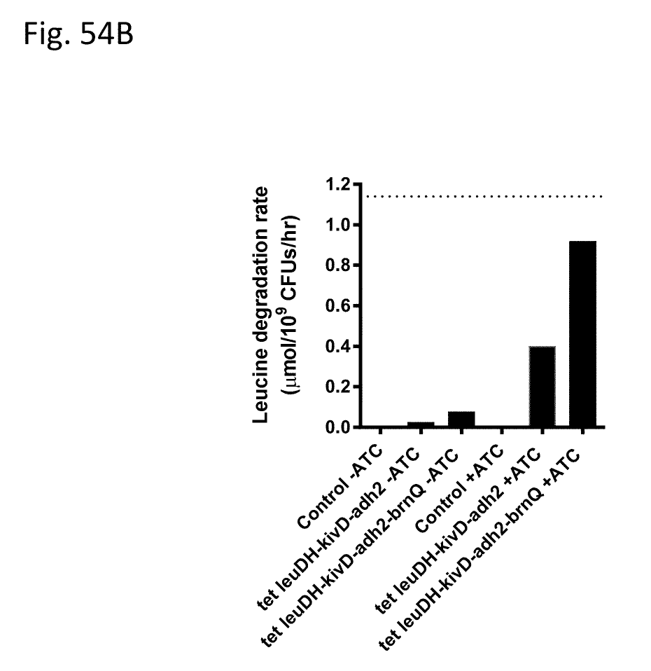

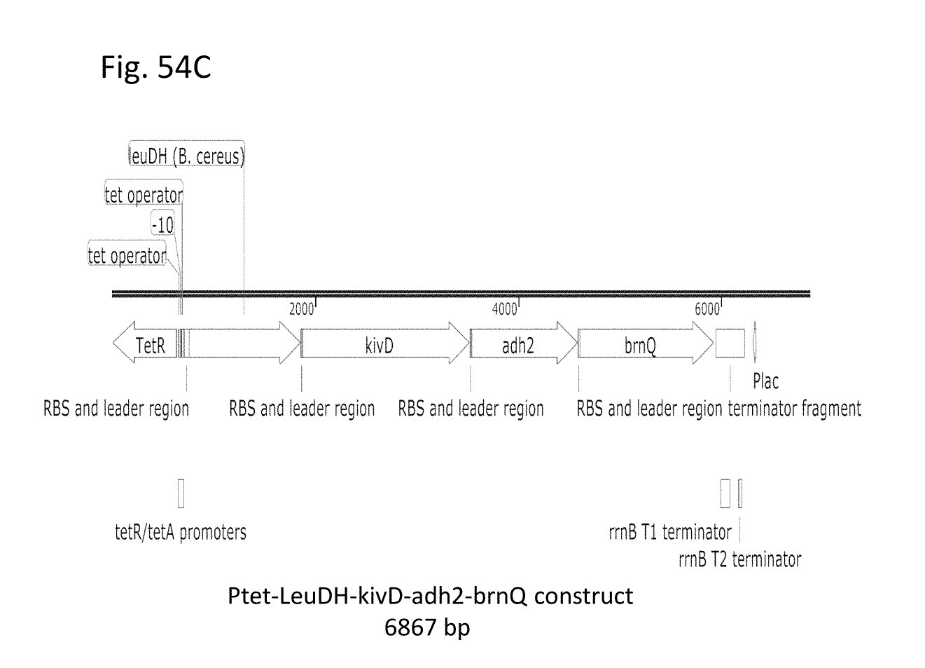

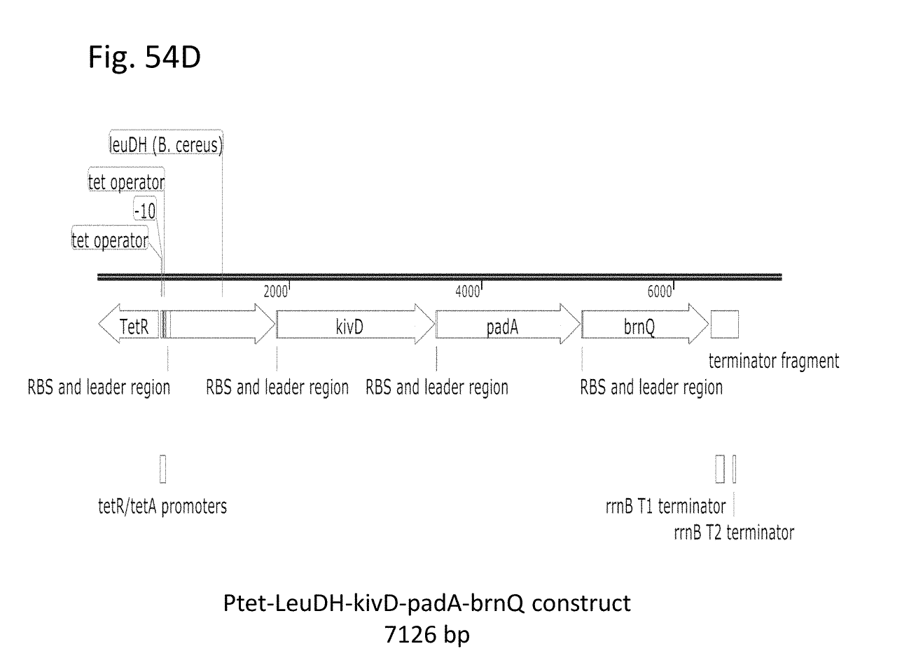

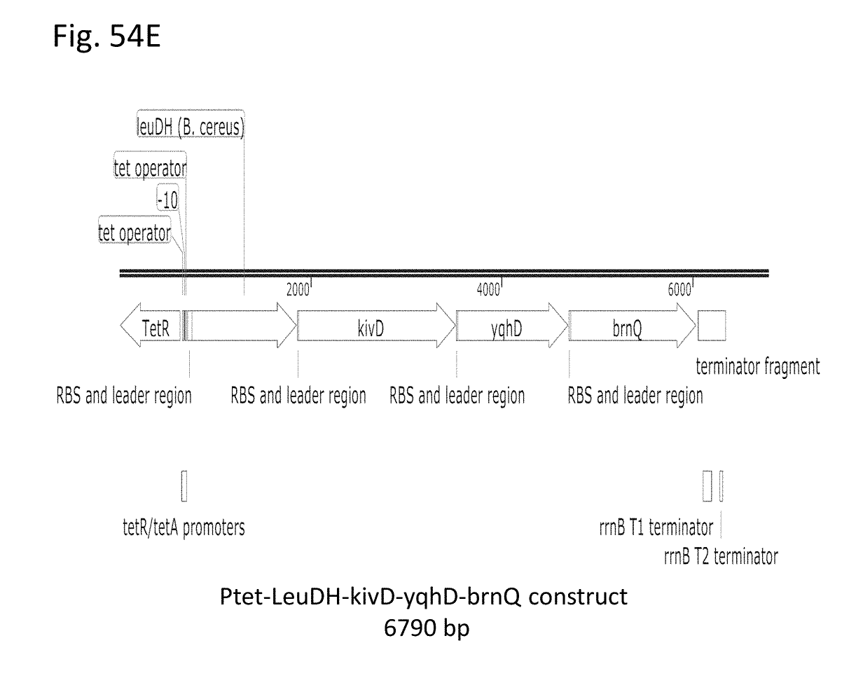

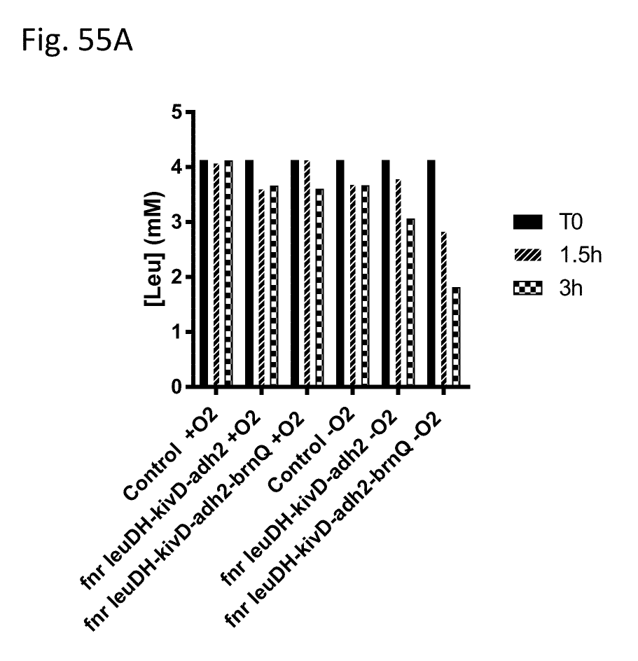

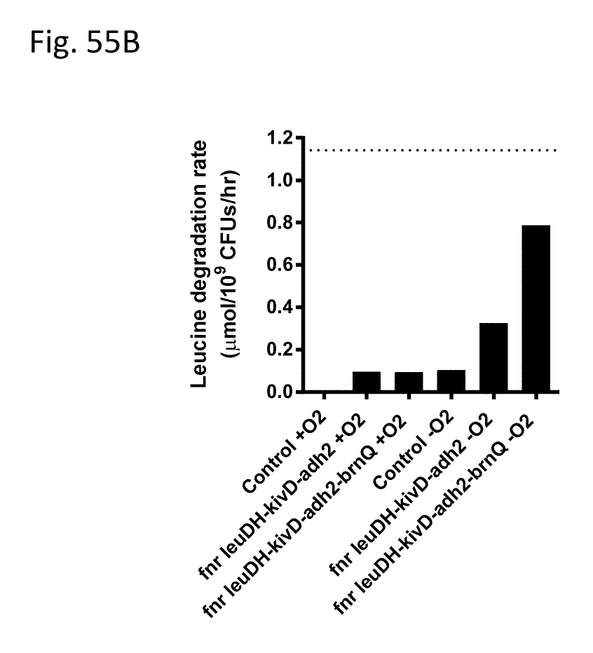

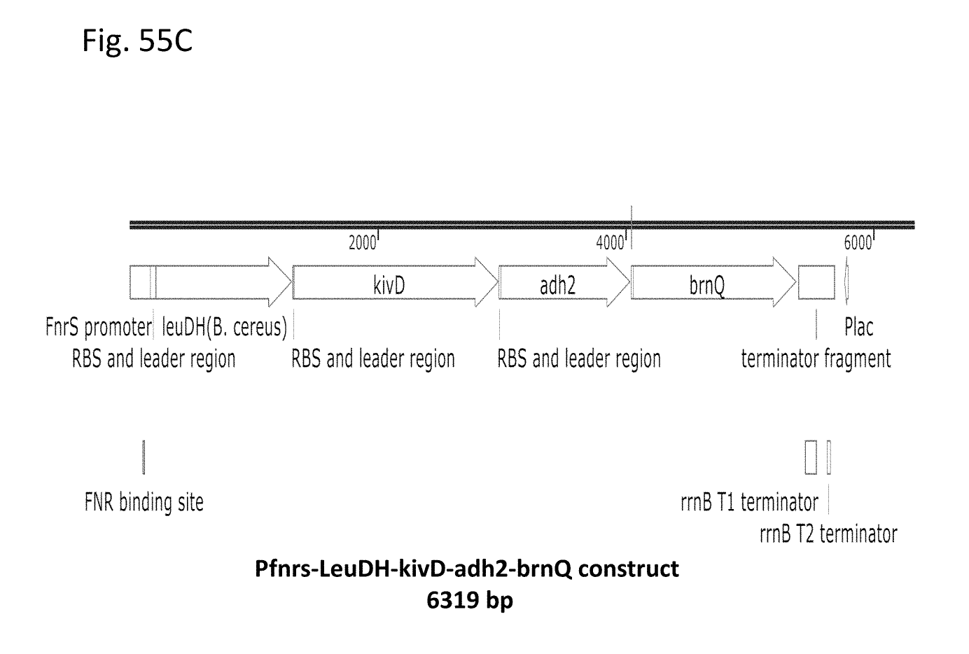

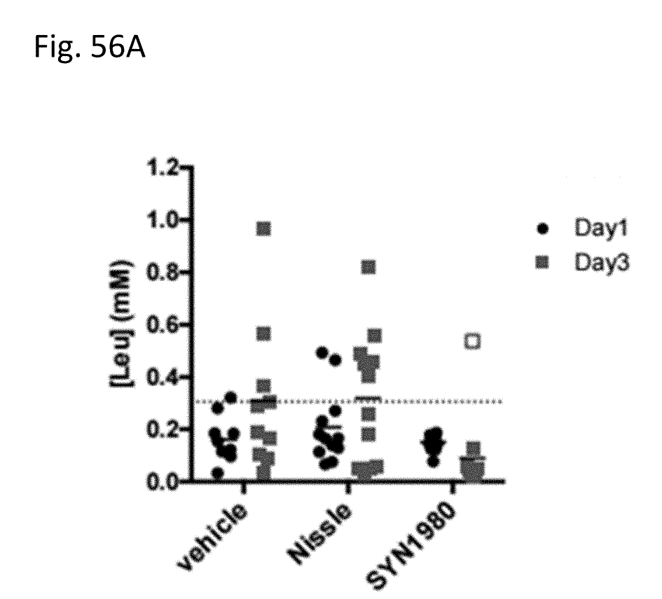

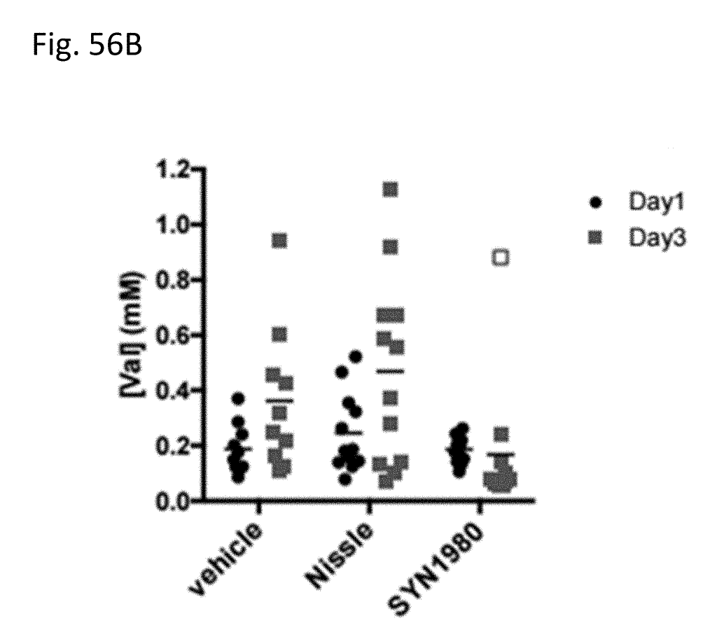

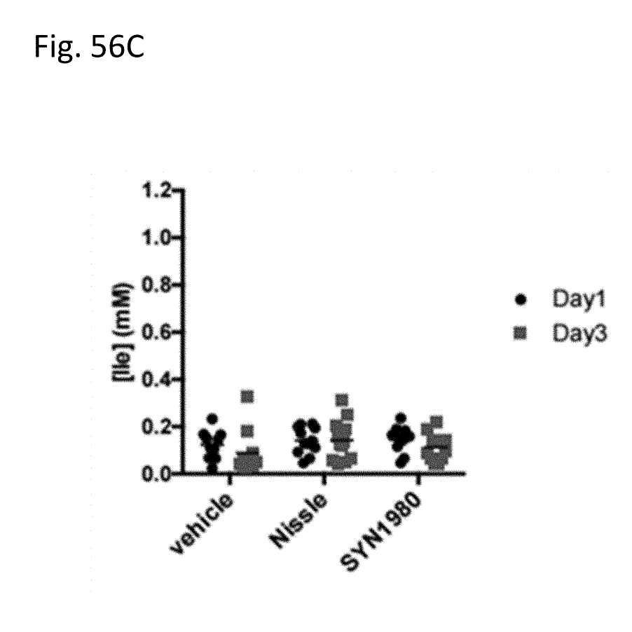

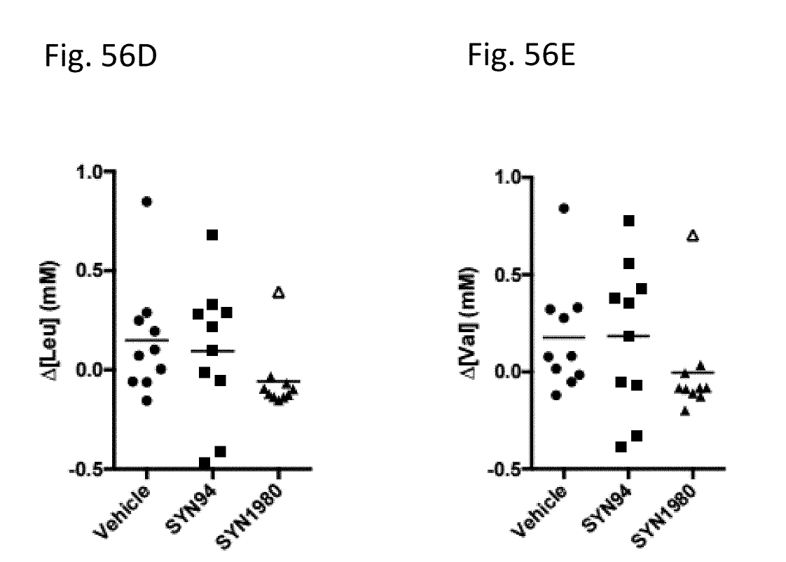

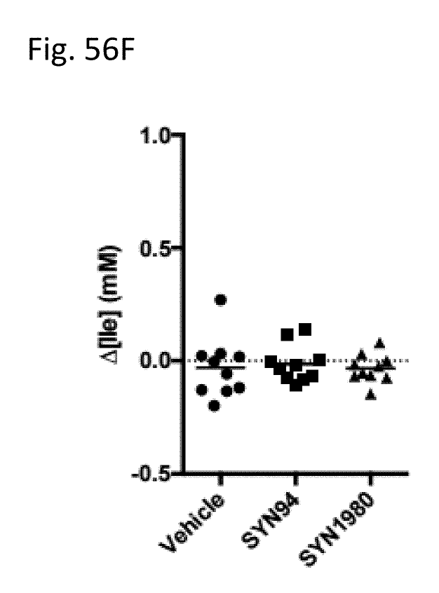

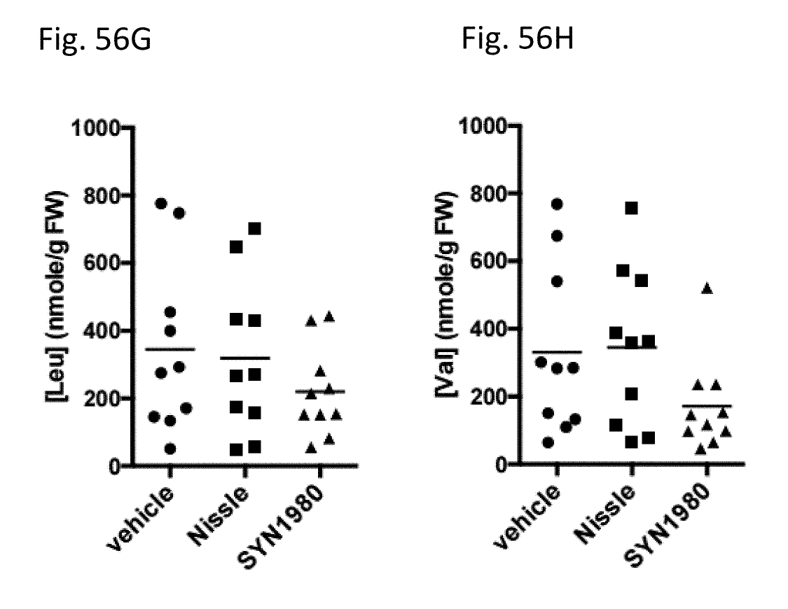

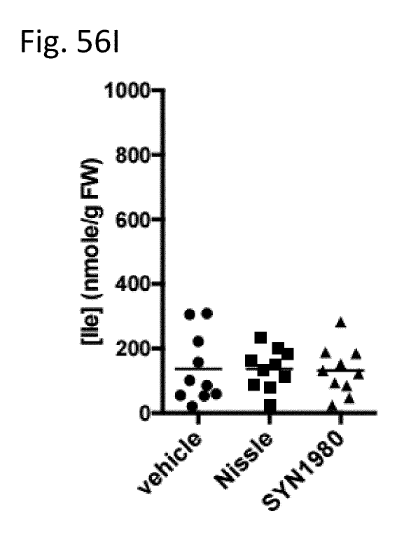

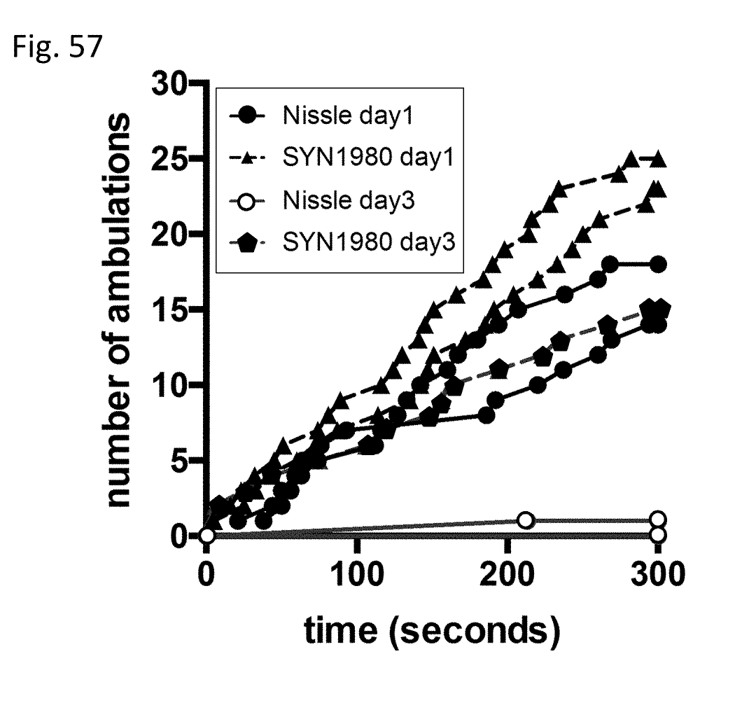

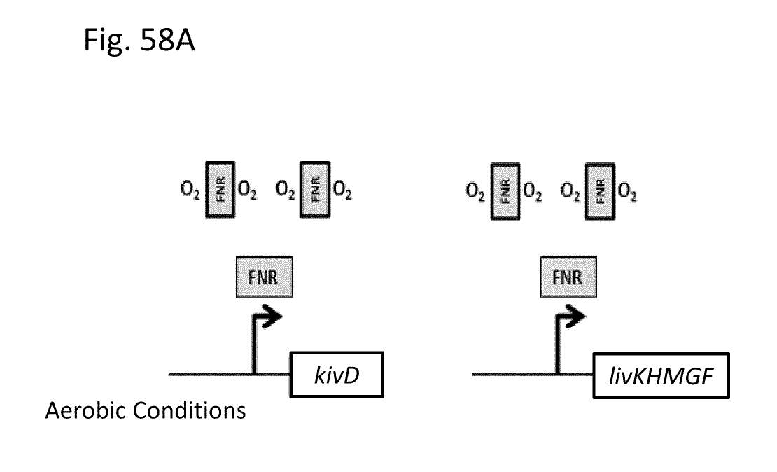

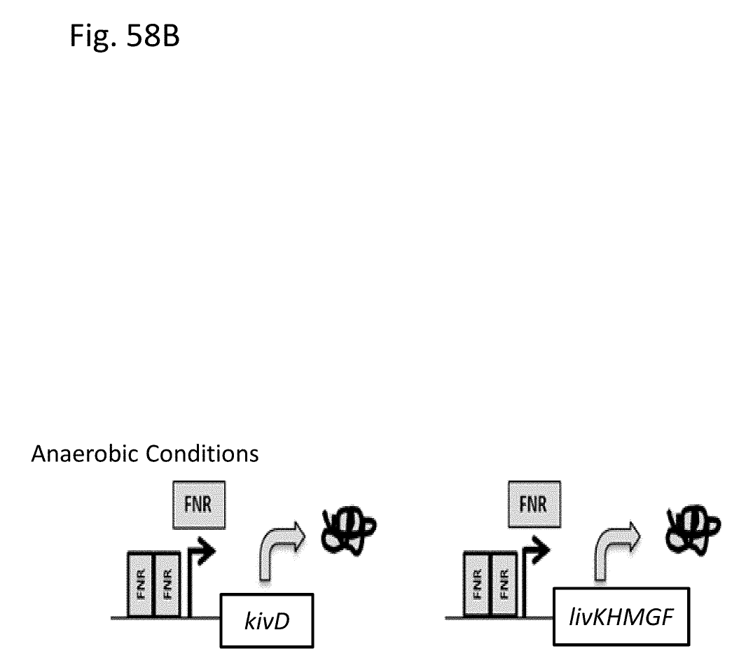

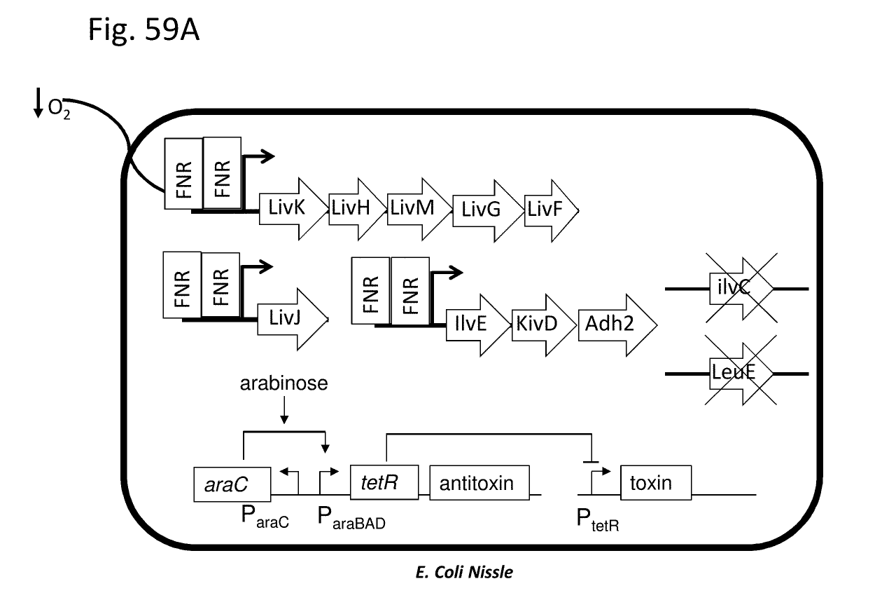

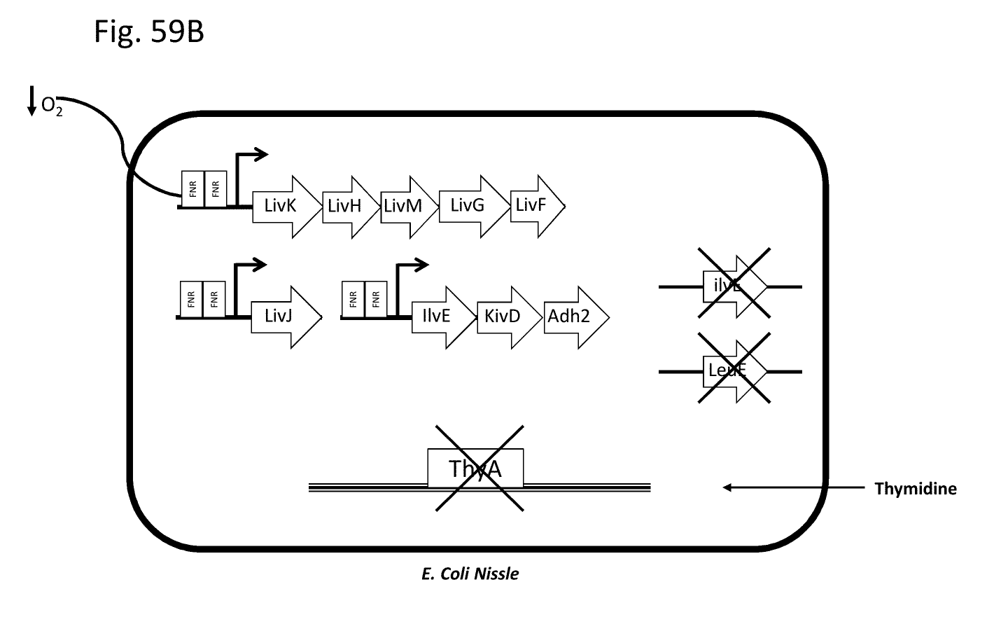

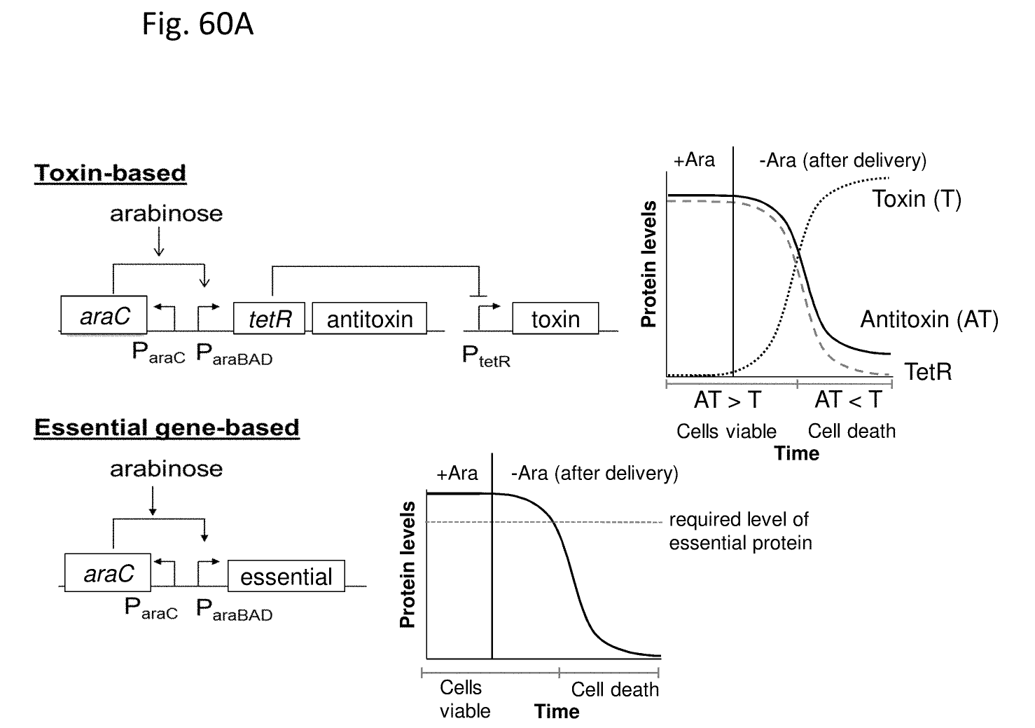

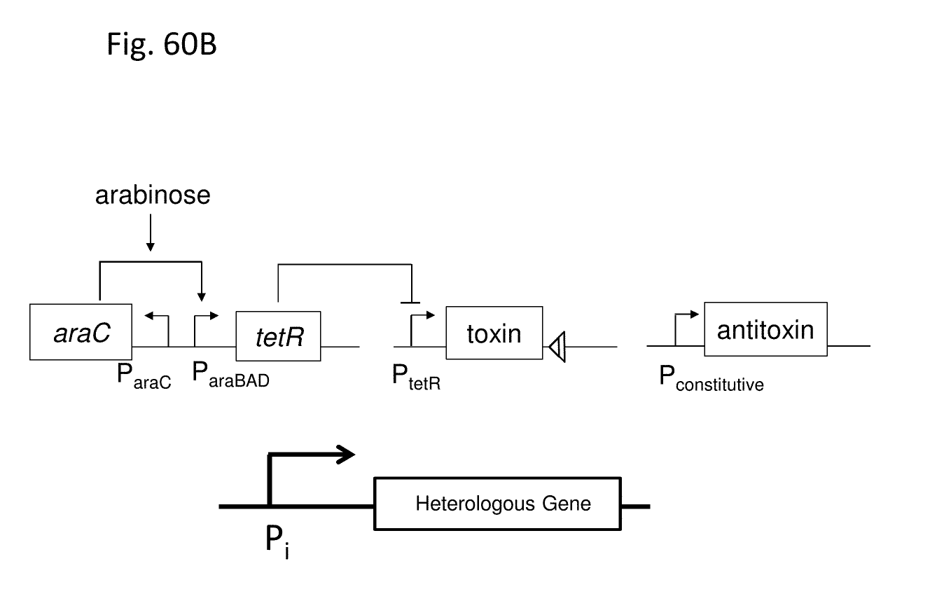

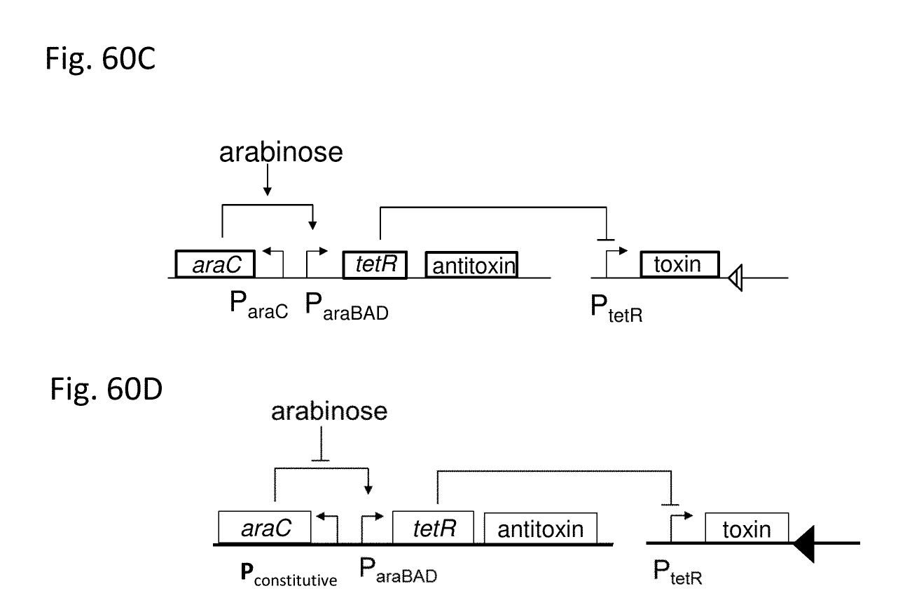

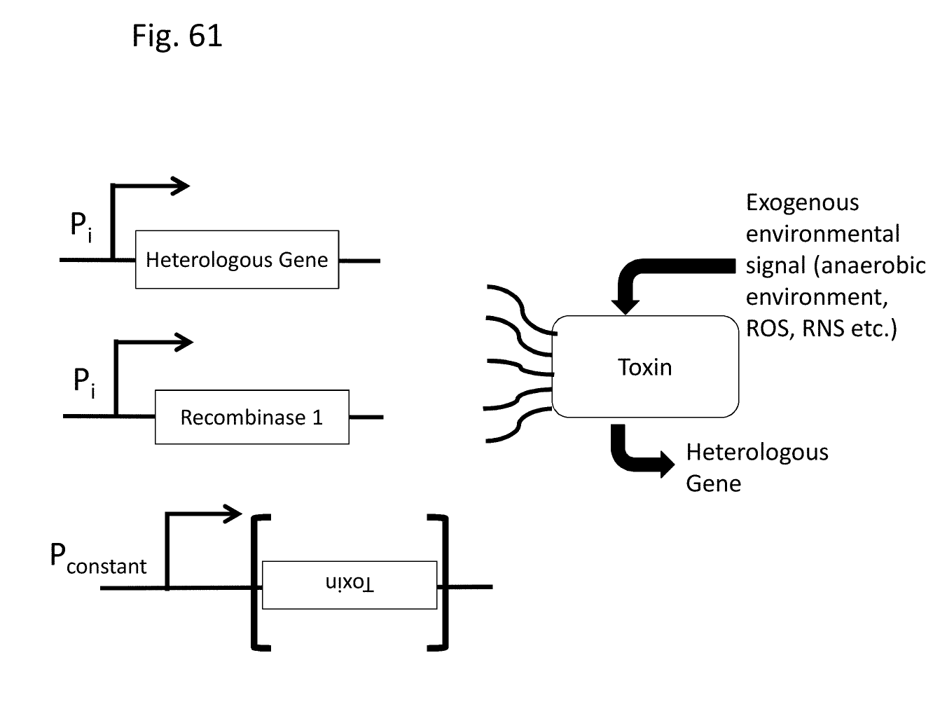

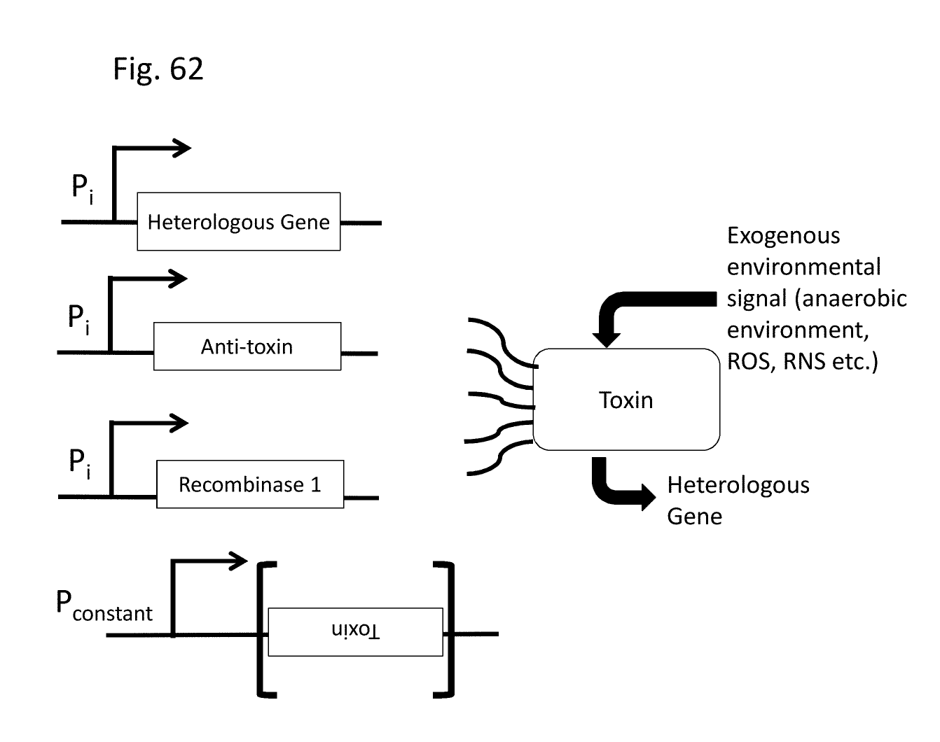

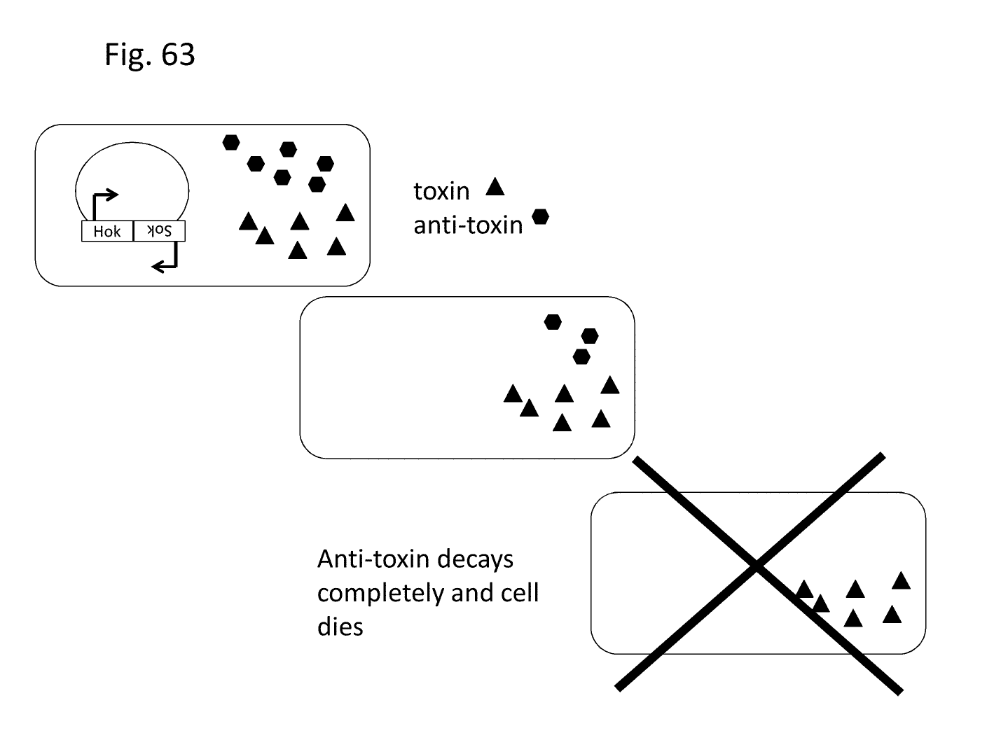

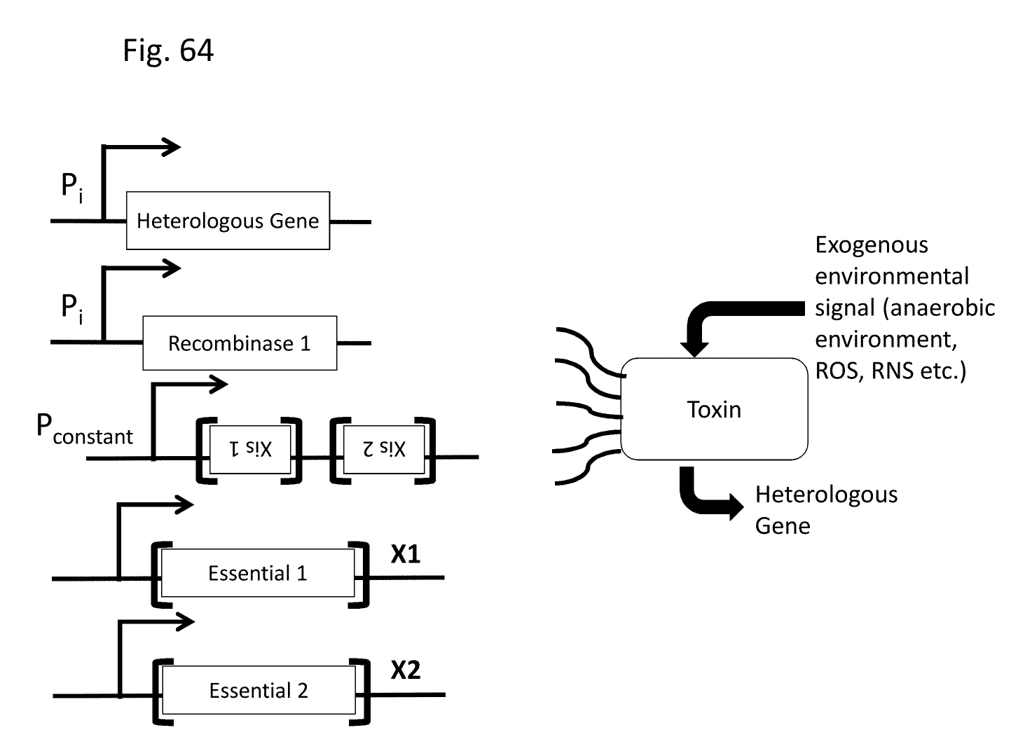

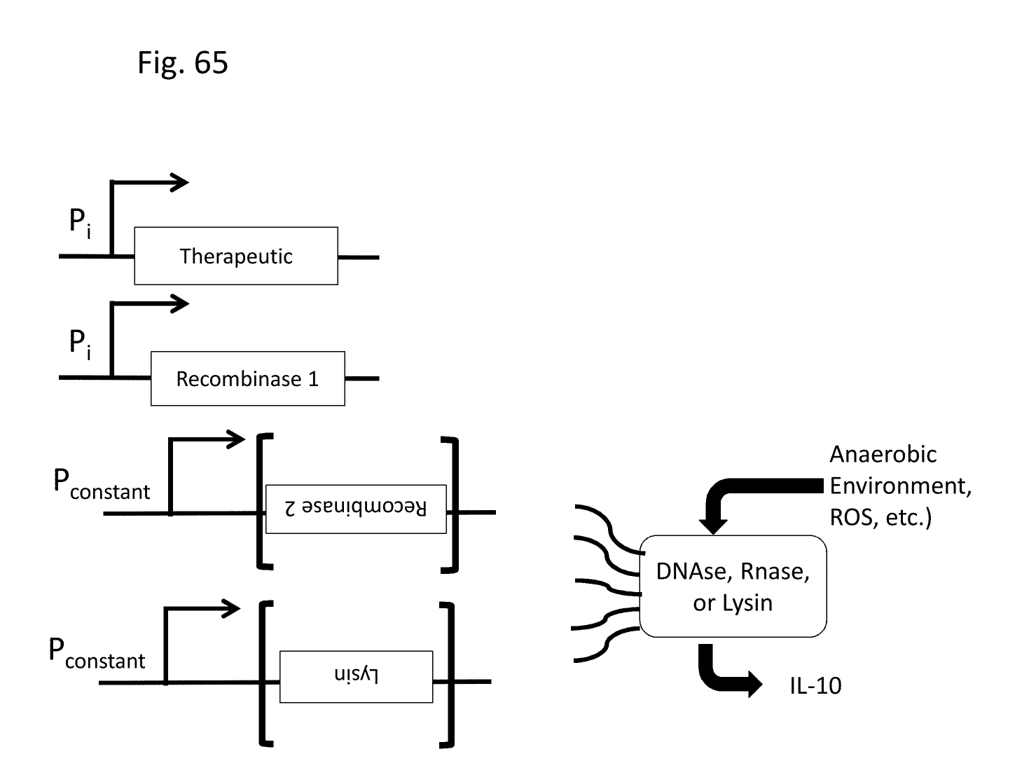

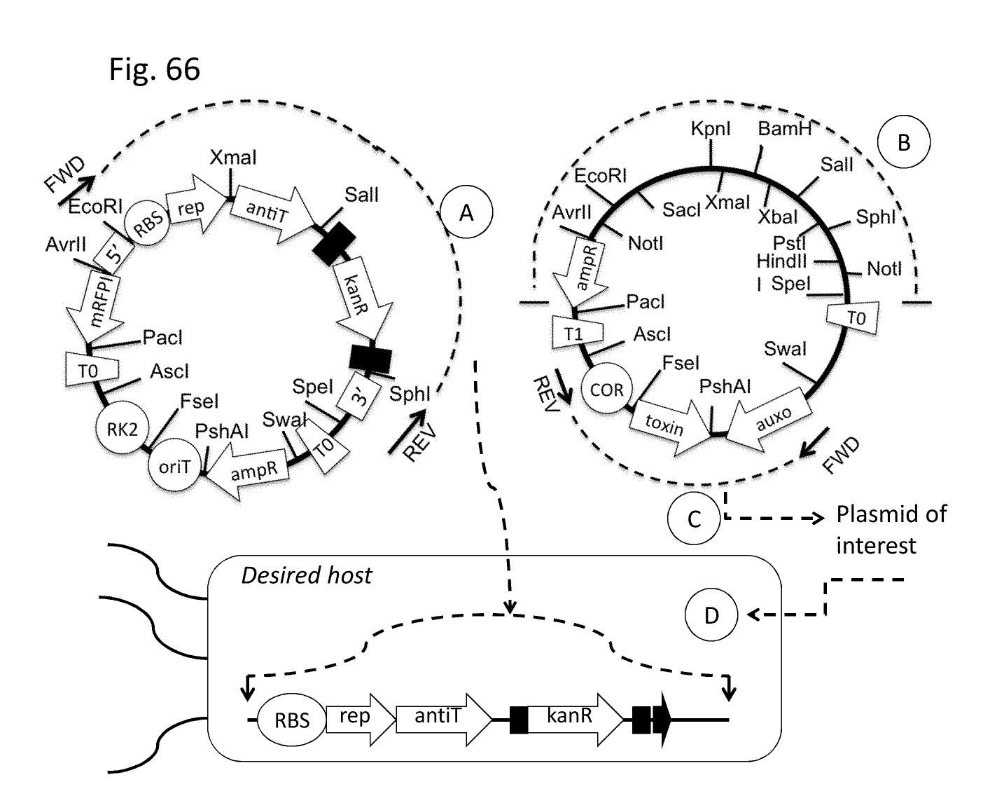

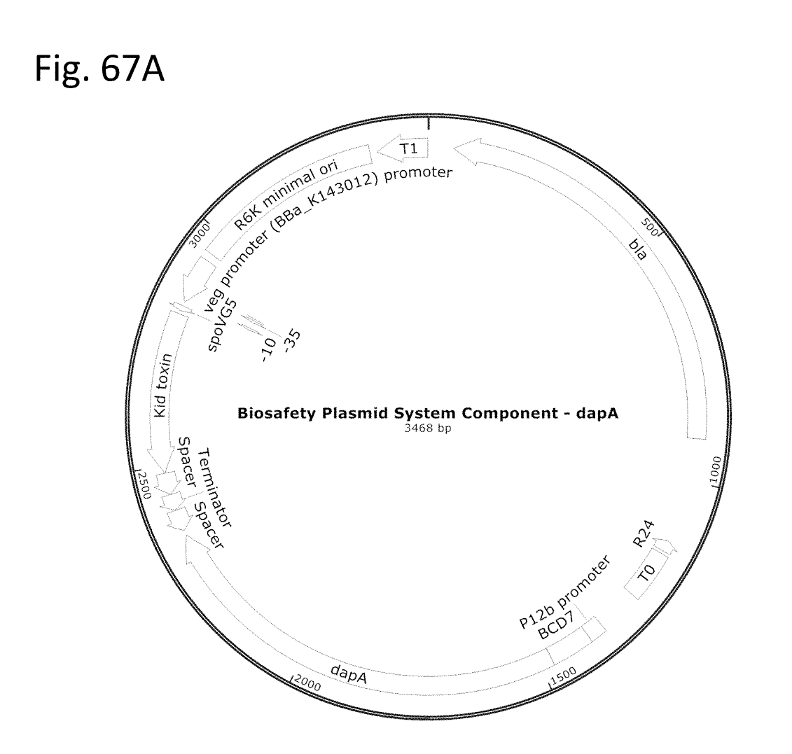

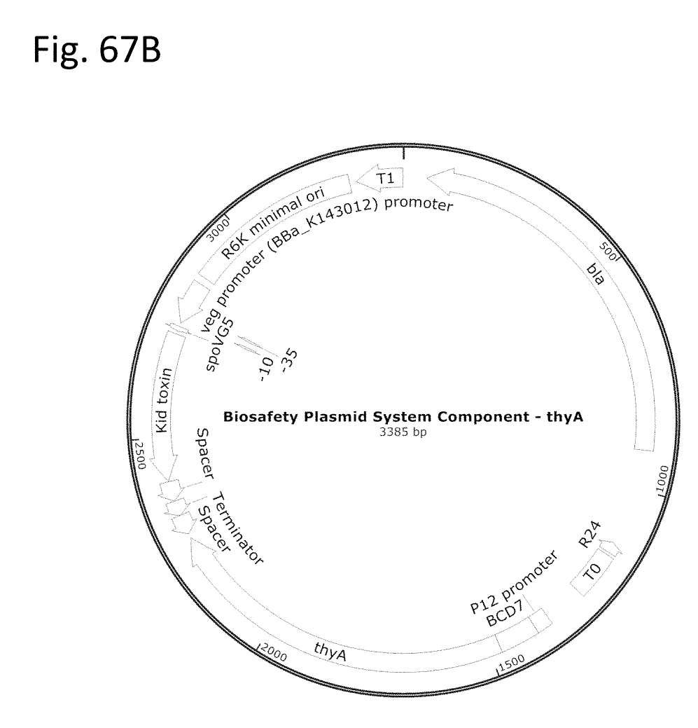

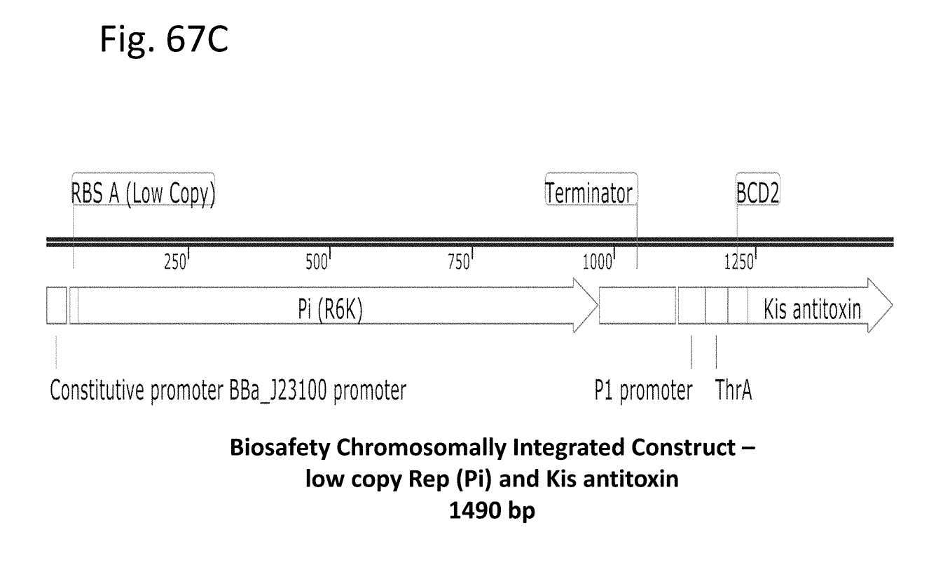

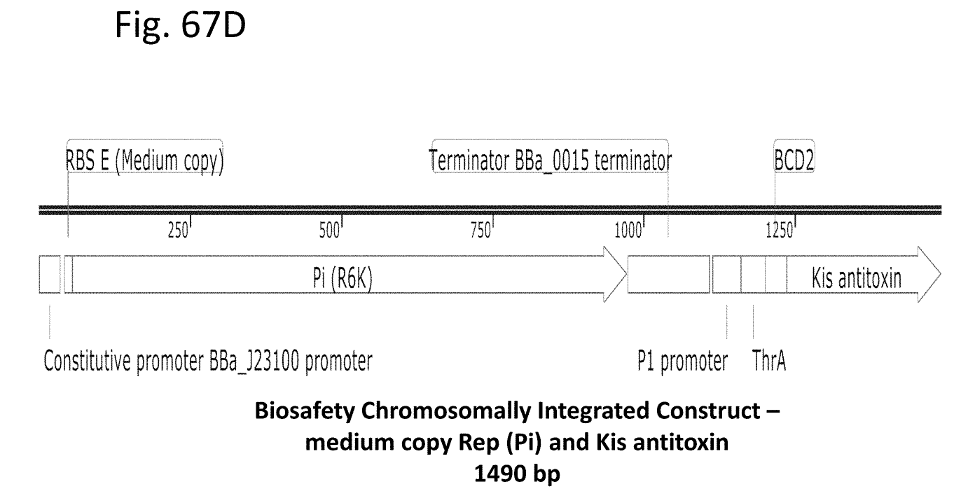

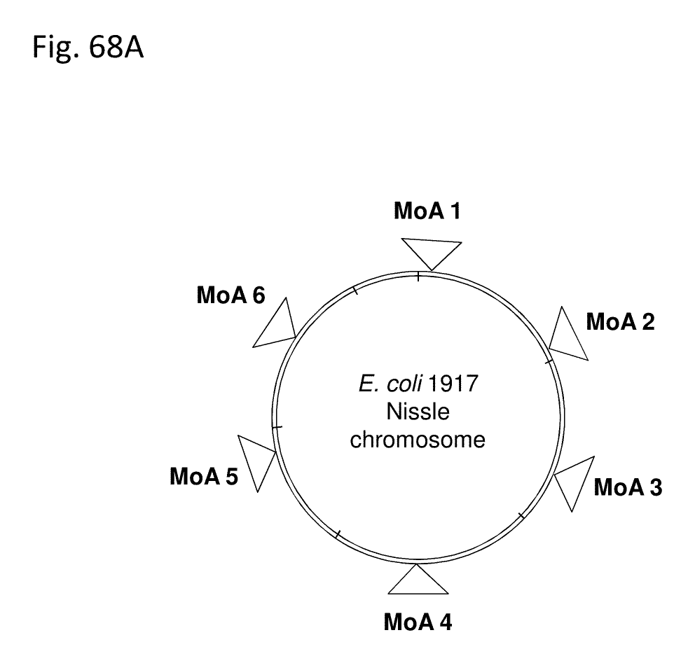

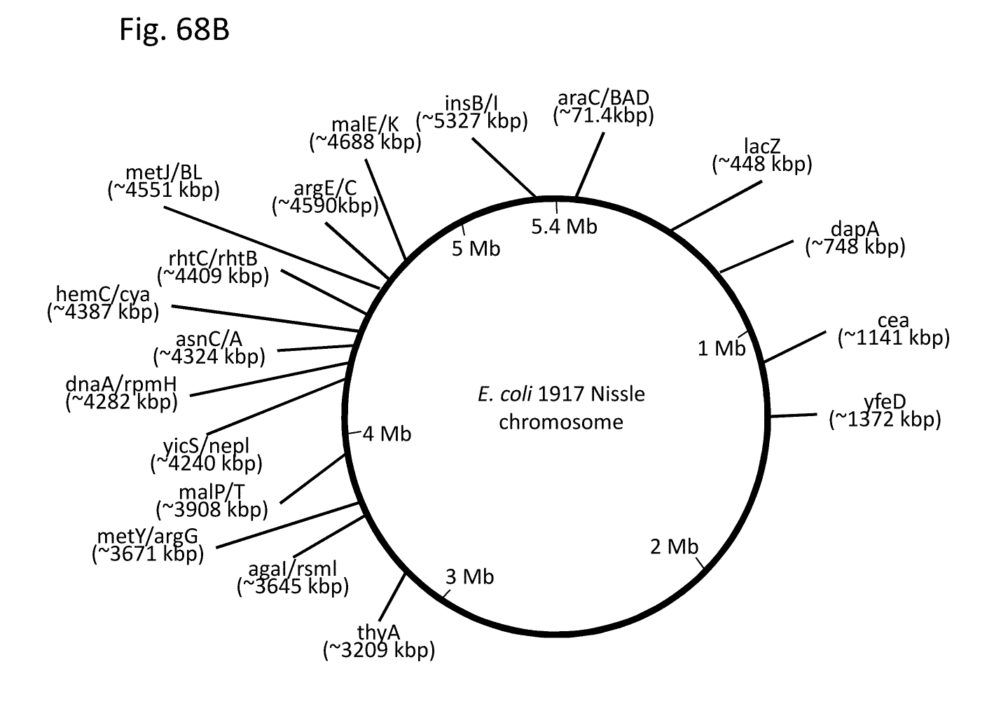

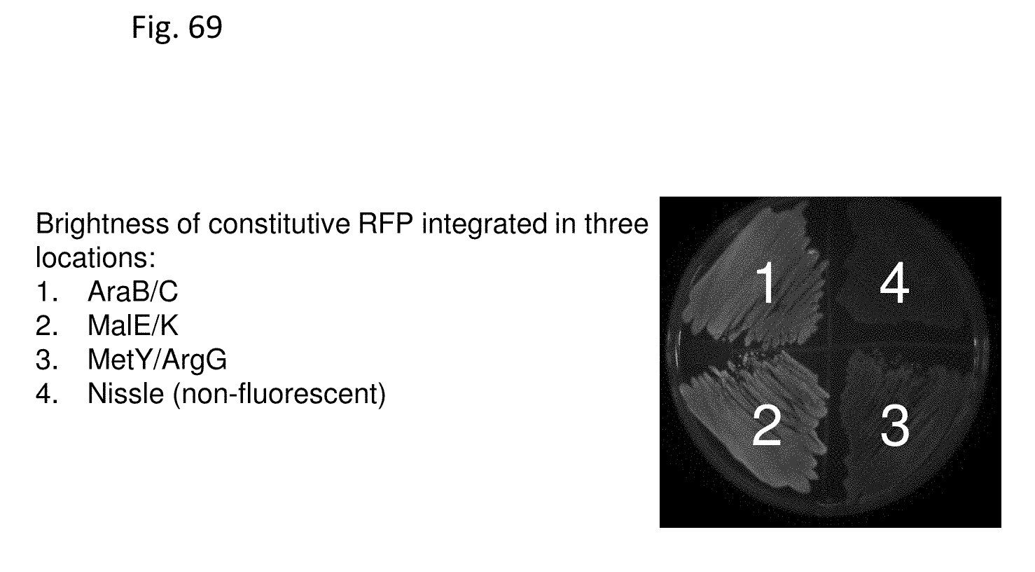

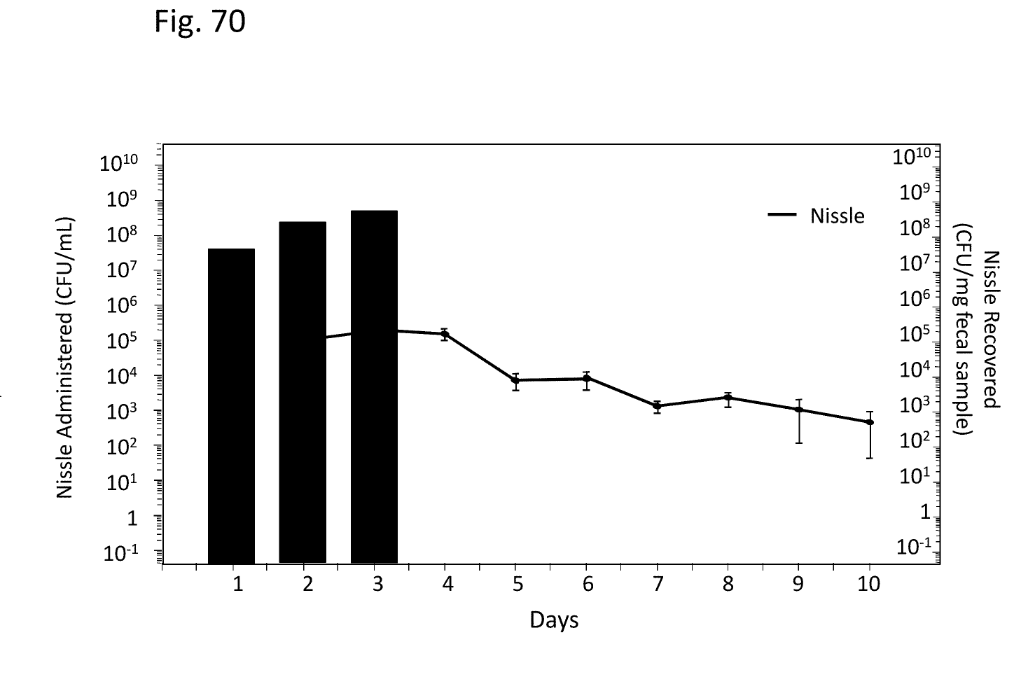

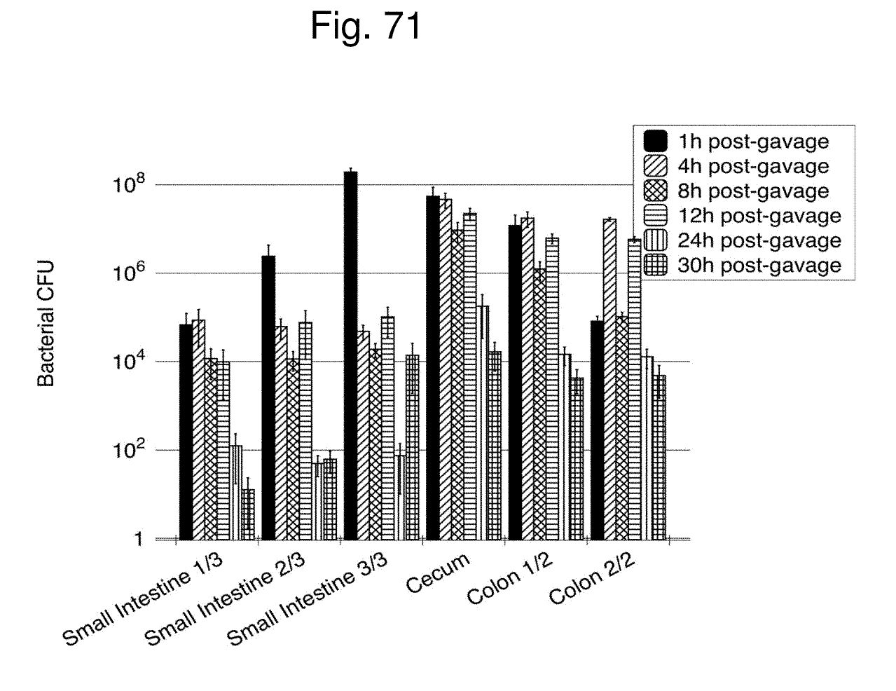

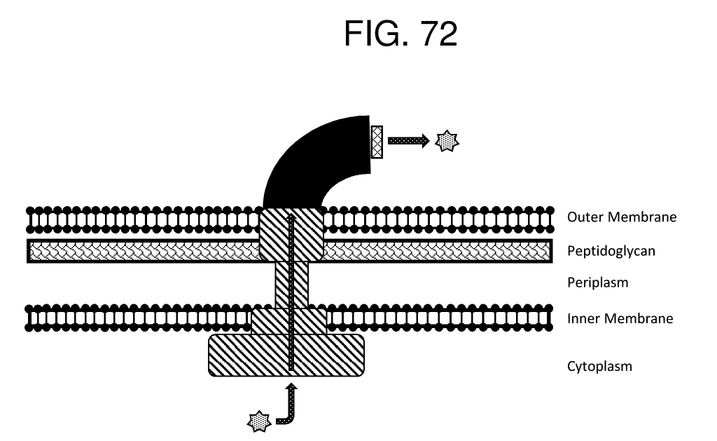

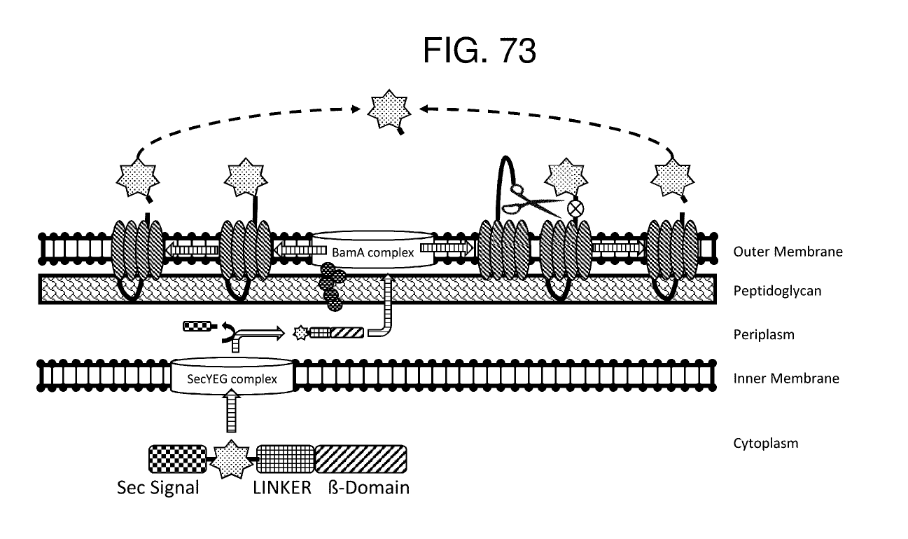

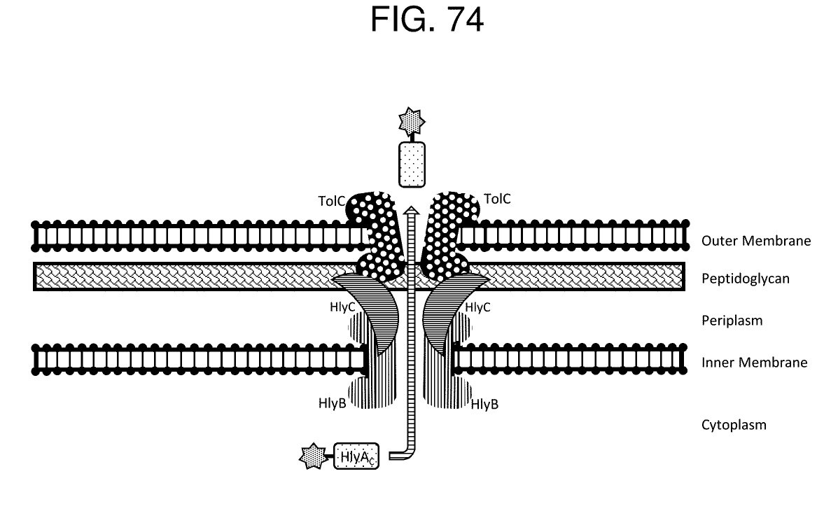

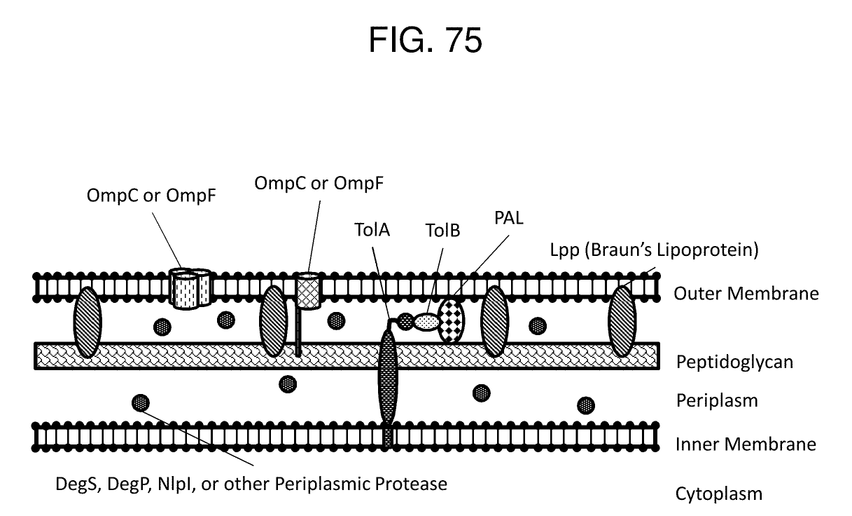

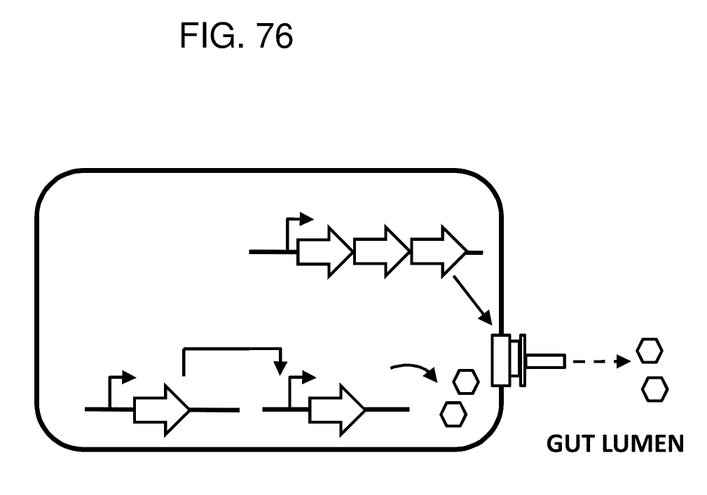

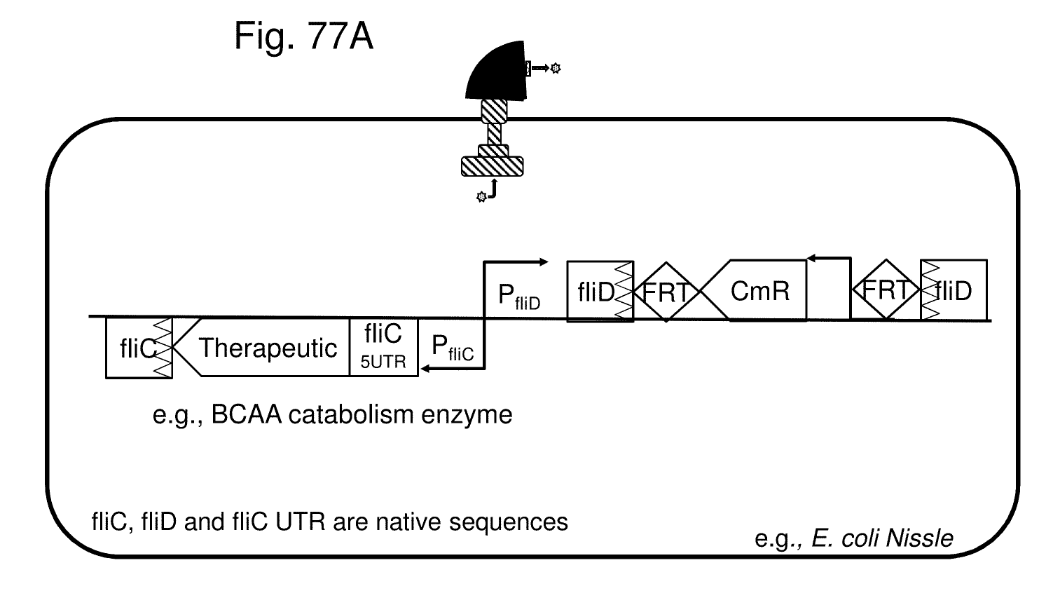

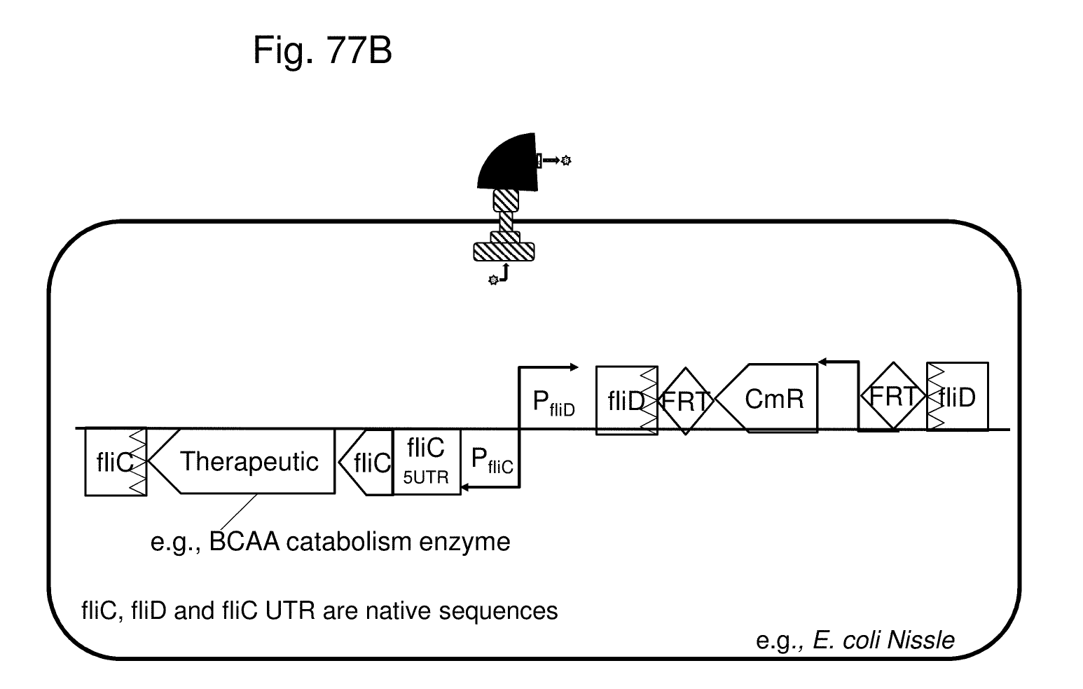

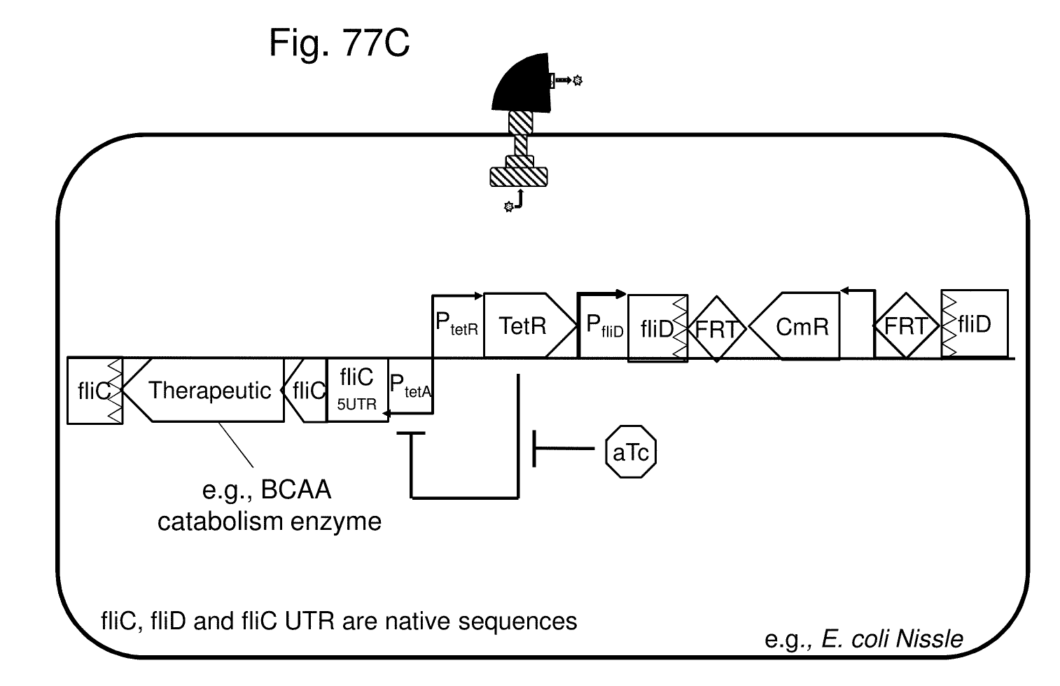

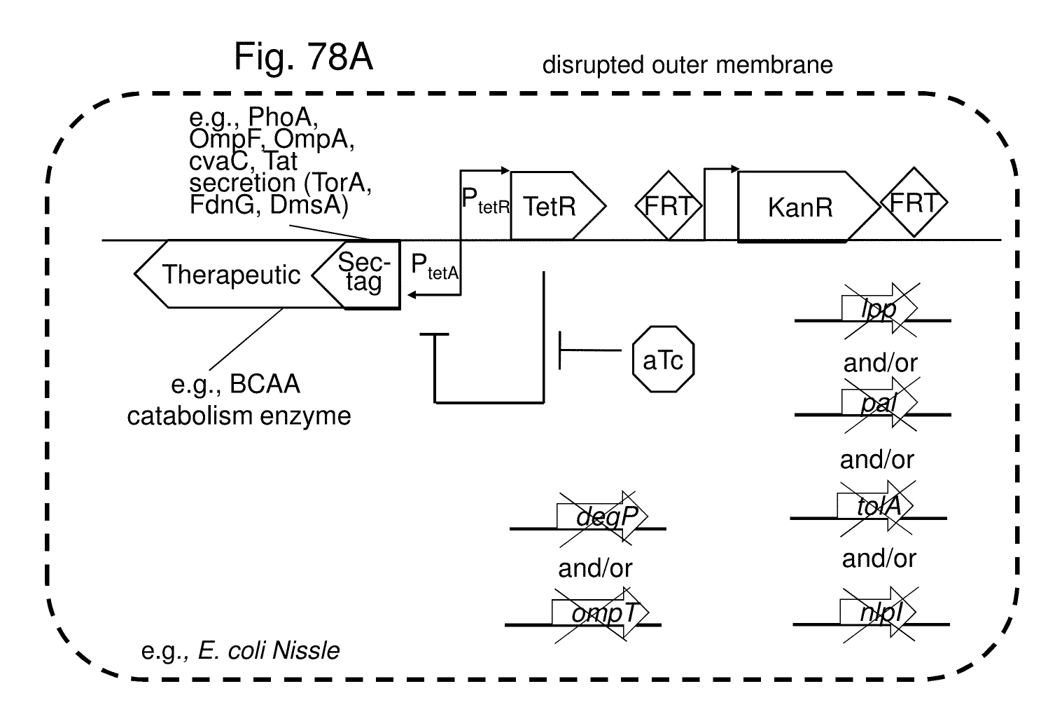

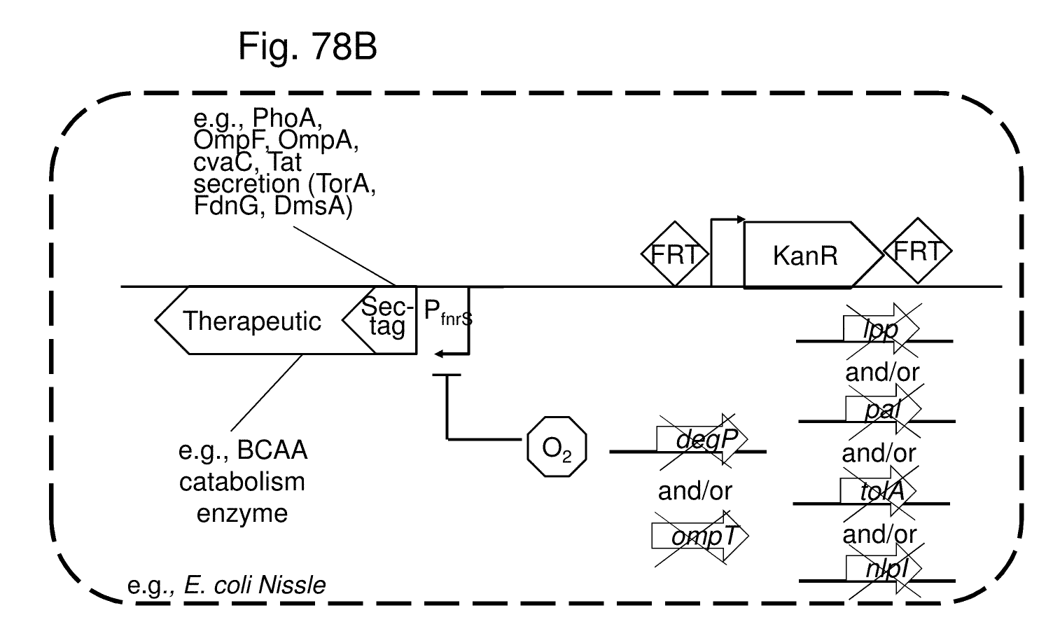

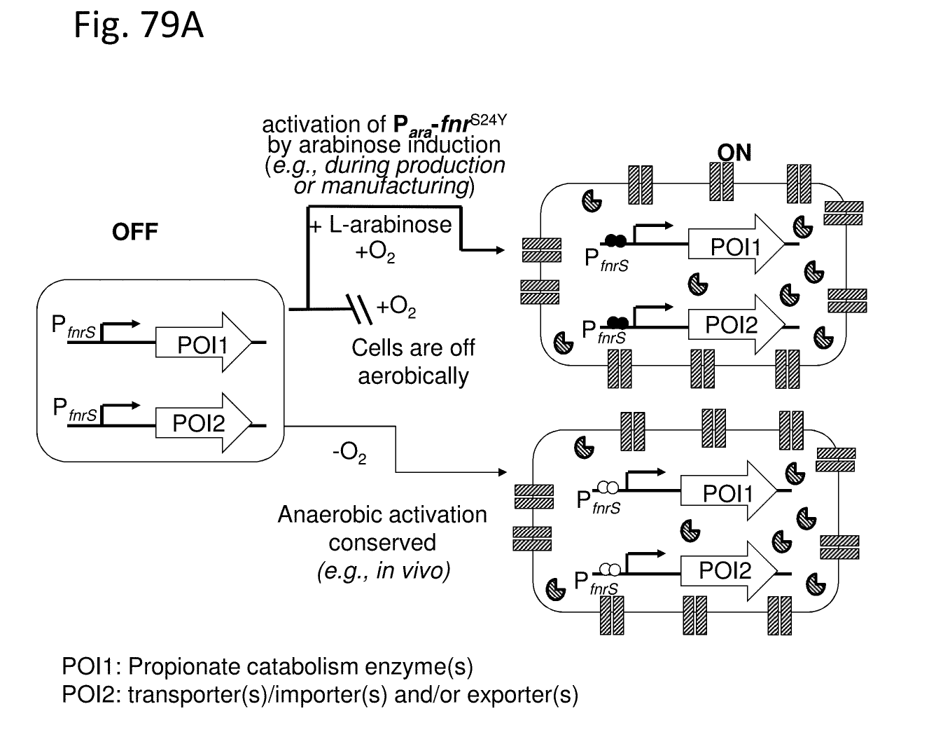

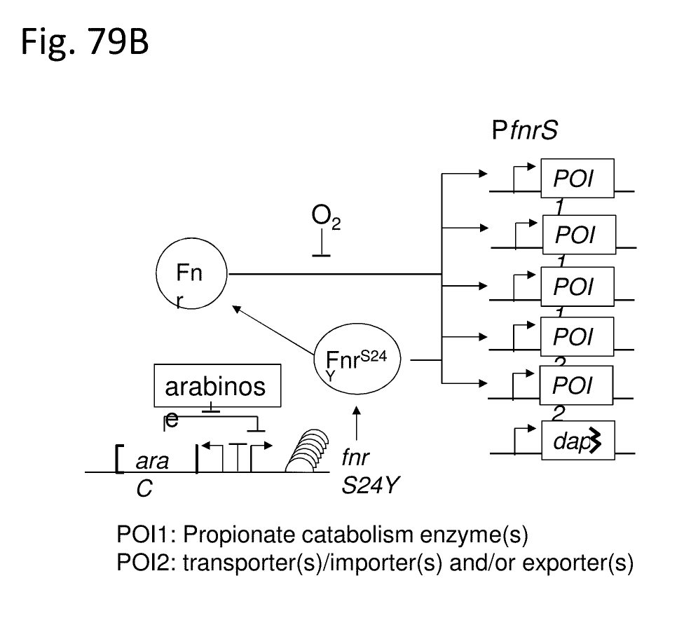

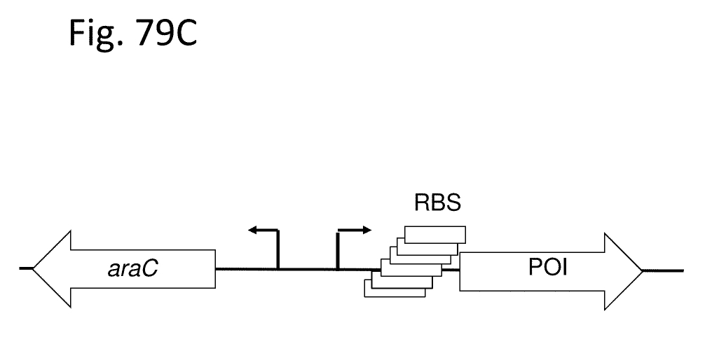

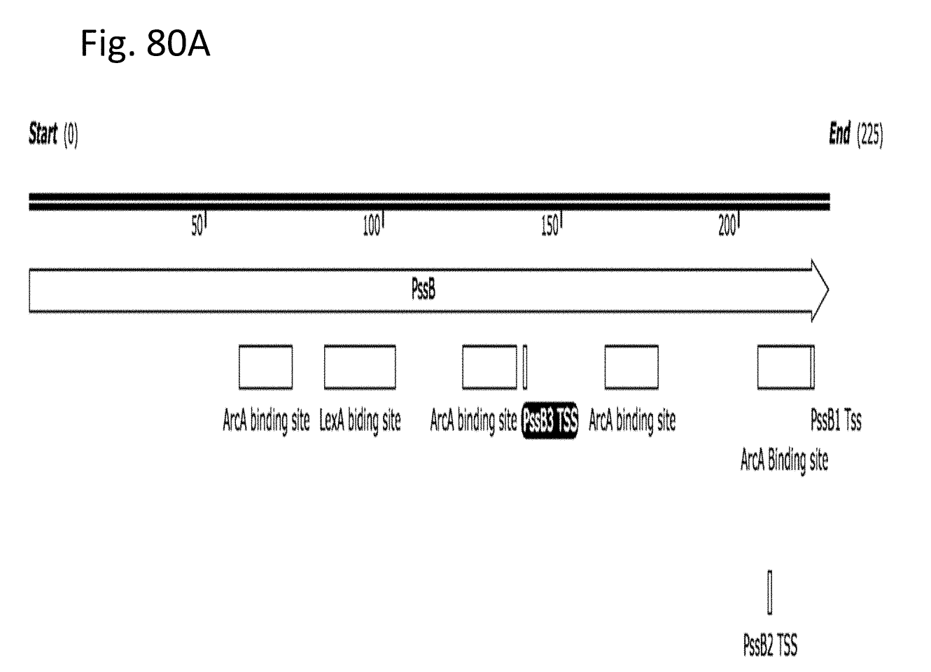

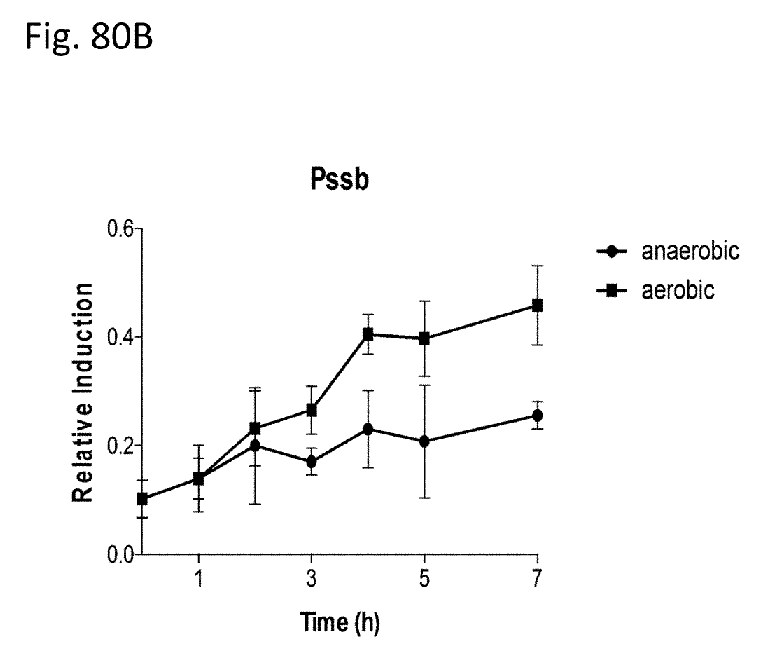

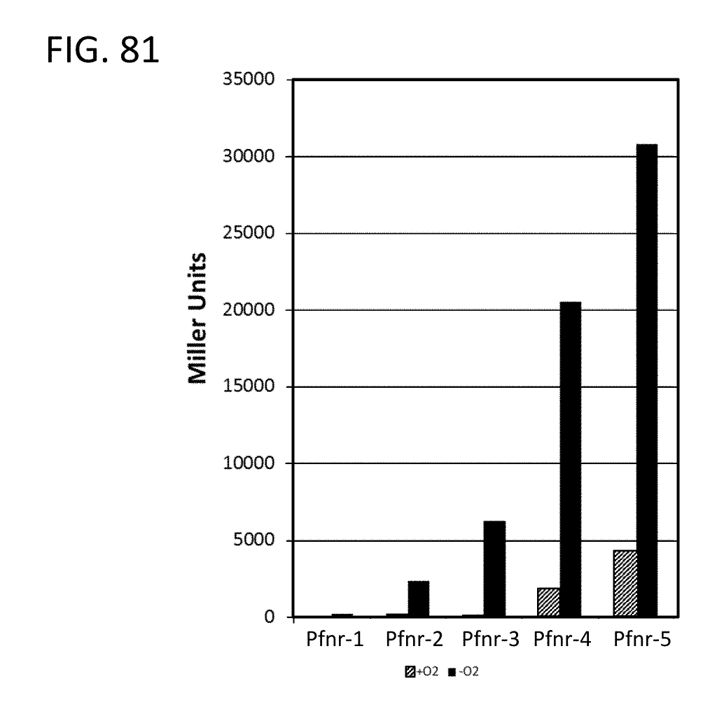

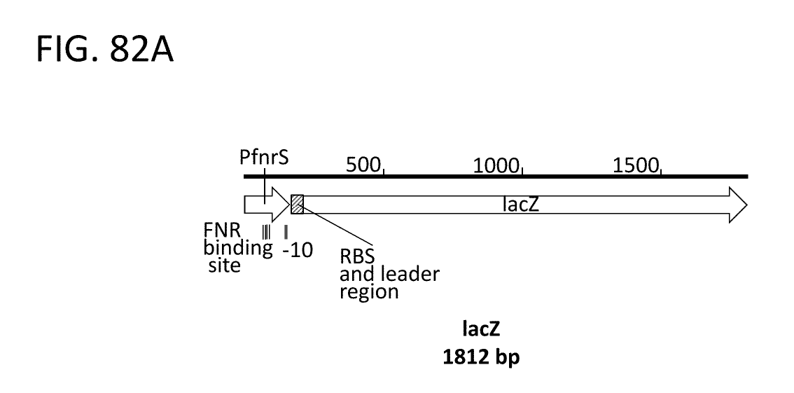

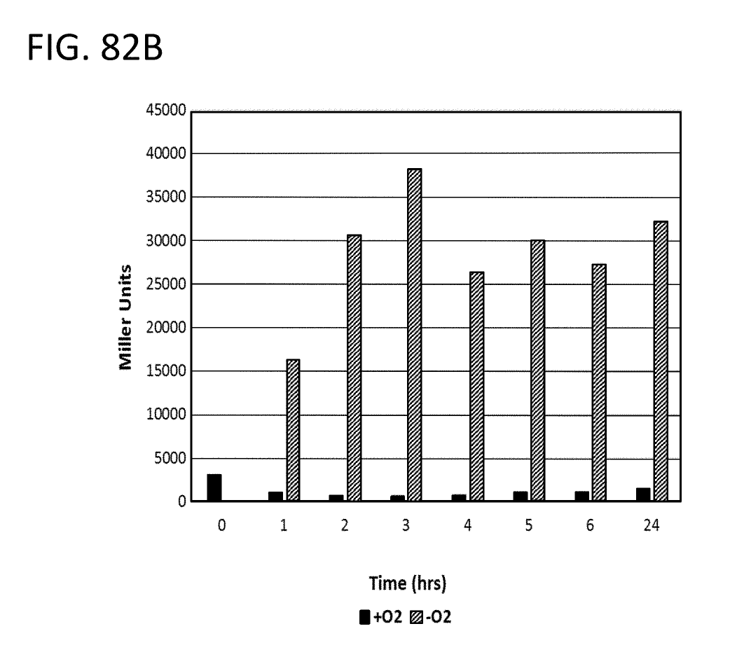

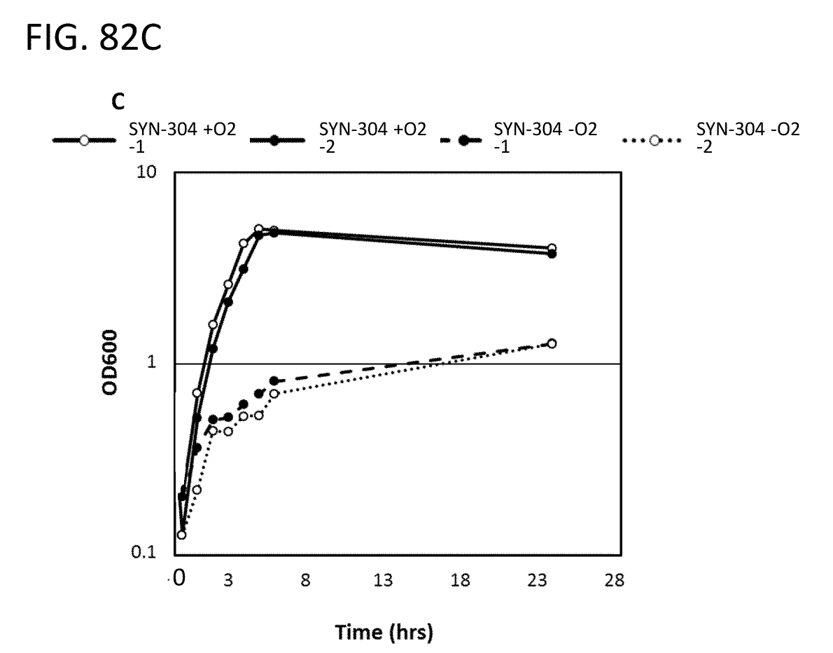

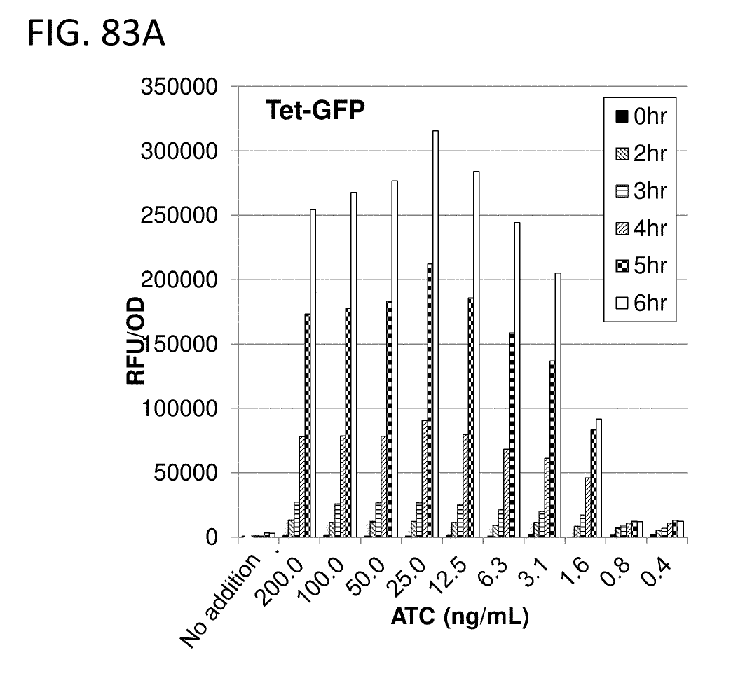

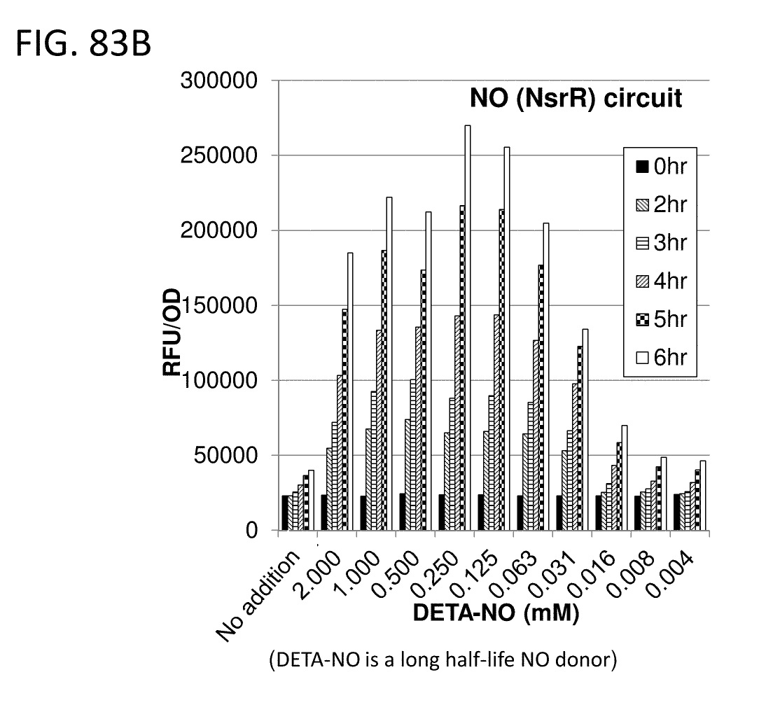

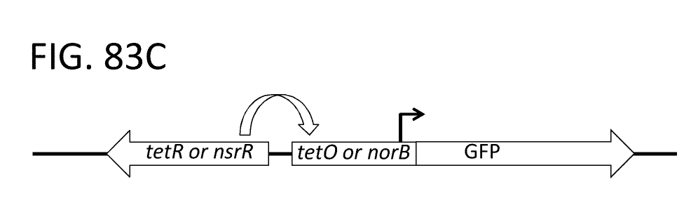

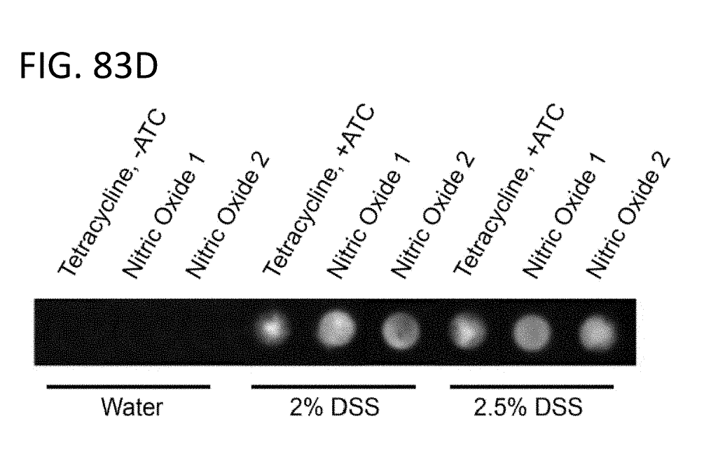

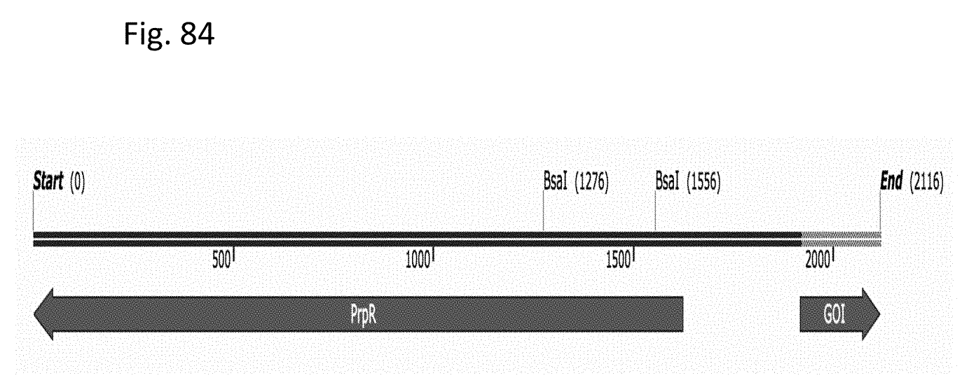

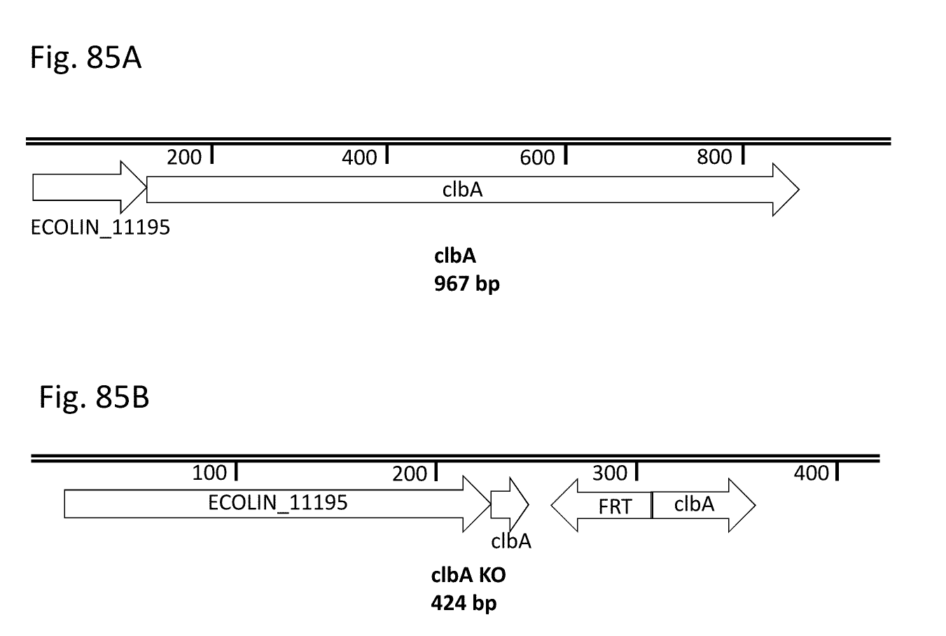

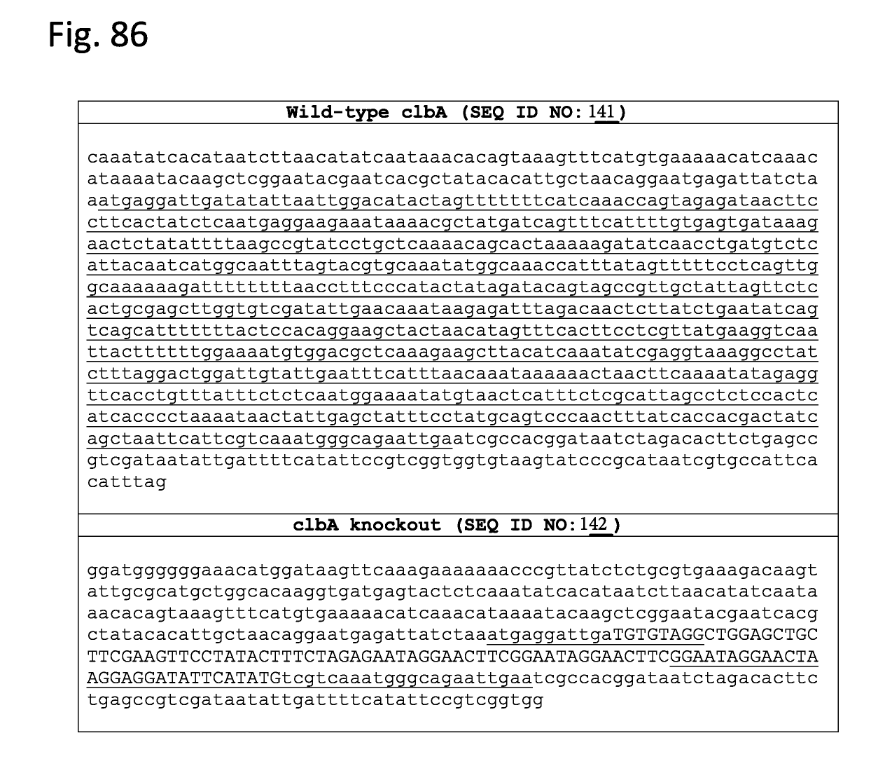

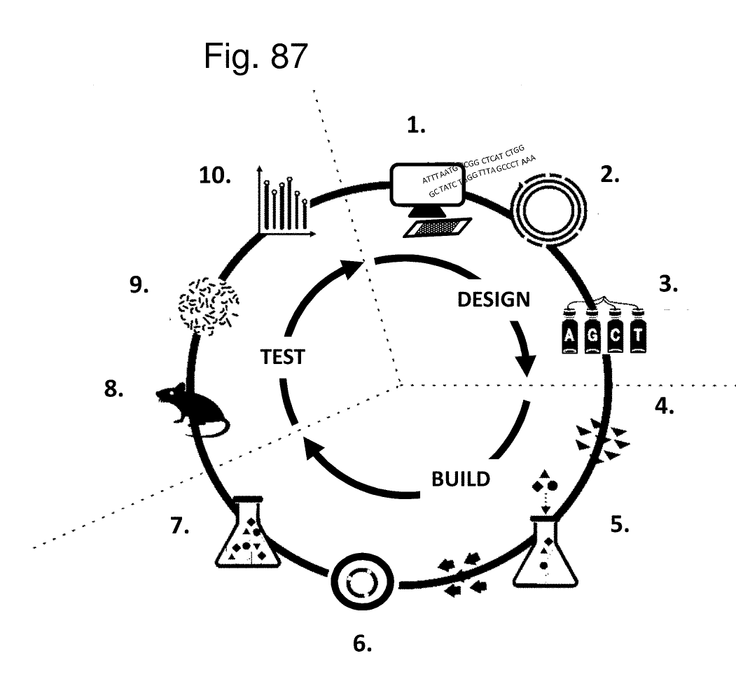

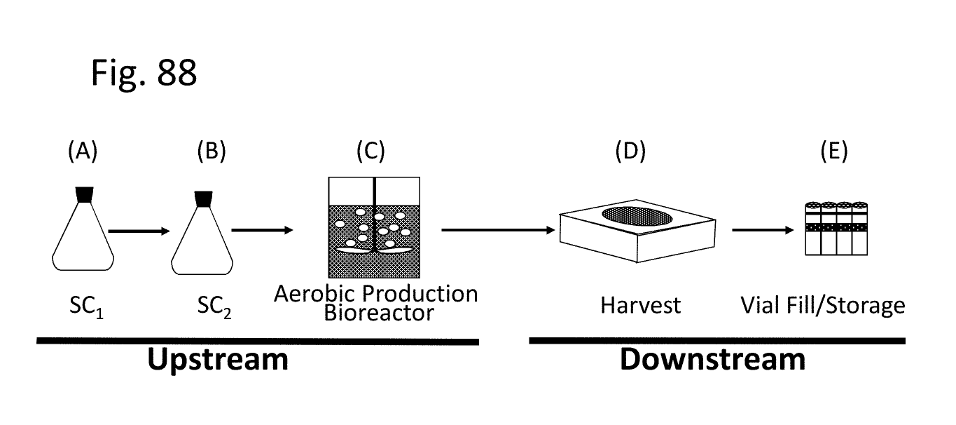

In some embodiments, combinations of two or more of the enzymes and/or enzyme complexes described herein may be secreted. Any of the enzymes expressed by the genes described e.g., in FIGS. 13A and 13B may be combined. Alternatively, any of the enzymes expressed by the genes described, e.g., or FIGS. 13D and/or E may be combined.

### Pharmaceutical Compositions and Formulations

Pharmaceutical compositions comprising the genetically engineered microorganisms of the invention may be used to treat, manage, ameliorate, and/or prevent a disorder associated with branched chain amino acid catabolism or symptom(s) associated with diseases or disorders associated with branched chain amino acid catabolism. Pharmaceutical compositions of the invention comprising one or more genetically engineered bacteria, and/or one or more genetically engineered yeast or virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise one or more of the genetic modifications described herein, e.g., selected from expression of at least one branched chain amino acid catabolism enzyme, BCAA transporter, BCAA binding protein, auxotrophy, kill-switch, exporter knock-out, etc. In alternate embodiments, the pharmaceutical composition comprises two or more species, strains, and/or subtypes of bacteria that are each engineered to comprise the genetic modifications described herein, e.g., selected from expression of at least one branched chain amino acid catabolism enzyme, BCAA transporter, BCAA binding protein, auxotrophy, kill-switch, exporter knock-out, etc.

The pharmaceutical compositions of the invention described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 104 to 1012 bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal

The genetically engineered bacteria or genetically engineered yeast or virus may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection.

The genetically engineered microorganisms of the disclosure may be administered intrathecally. In some embodiments, the genetically engineered microorganisms of the invention may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered viruses are prepared for delivery, taking into consideration the need for efficient delivery and for overcoming the host antiviral immune response. Approaches to evade antiviral response include the administration of different viral serotypes as par of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In Vivo Methods

The recombinant bacteria disclosed herein may be evaluated in vivo, e.g., in an animal model. Any suitable animal model of a disease or condition associated with catabolism of a branched chain amino acid may be used (see, e.g., Skvorak, J. Inherit. Metab. Dis., 2009, 32:229-246 and Homanics et al., BMC Med. Genet., 2006, 7(33):1-13), including the Dbt−/− model (E2 subunit of BCKDH, which has a 3-fold increase in blood and urine BCAA levels and results in neonatal lethalthy) (serves as classic MSUD model). This model is partially rescued by two transgenes (LAP-tTA and TRE-E2), allowing 5-6% of normal BCKDH activity and an increase in mice survival to three or four weeks (serves as an intermediate MSUD model) (as described in Homanics et al., 2006, the contents of which is herein incorporated by reference in its entirety). In addition, intermediate MSUD mice can be used to to show development of neuropathology with striking similarity to human MSUD. In this model, branched-chain amino acid accumulation was associated with neurotransmitter deficiency, behavioral changes and limited survival, and providing intermediate MSUD mice with a choice between normal and branched-chain amino acidfree diet prevented brain injury and dramatically improved survival (Zinnanti et al., Dual mechanism of brain injury and novel treatment strategy in maple syrup urine disease; Brain 2009: 132; 903-918, the contents of which is herein incorporated by reference in its entirety). In some embodiments, the animal model is a mouse model of Maple Syrup Urine Disease. In one embodiment, the mouse model of MSUD is a branched-chain amino transferase knockout mouse (Wu et al., J. Clin. Invest, 1B:434-440, 2004 or She et al., Cell Metabol., 6:181-194, 2007). In another embodiment, the mouse model of MSUD is a dihydrolipoamine dehydrogenase (E3) subunit knock-out mouse (Johnson et al., Proc. Natl. Acad. Sci. U.S.A., 94:14512-14517, 1997). In another embodiment, the mouse model of classic MSUD is a deletion of exon 5 and part of exon 4 of the E2 subunit of the branched-chain alpha-keto acid dehydrogenase (Homanics et al., BMC Med. Genet., 7:33, 2006) or the mouse model of intermediate MSUD (Homanics et al., BMC Med. Genet., 7:33, 2006). In another embodiment, the model is a Polled Shorthorn calf model of disease or a Polled Hereford calf model of disease (Harper et al., Aus. Vet. J., 66(2):46-49, 1988). Other relevant animal models include those described in She et al., Cell Metab. 2007 September; 6(3): 181-194; Wu et al., J. Clin. Invest. 113:434-440 (2004); Bridi, et al., J Neurosci Methods. 2006 Sep. 15; 155(2):224-30.

The recombinant bacterial cells disclosed herein may administered to the animal, e.g., by oral gavage, and treatment efficacy is determined, e.g., by measuring blood leucine levels before and after treatment. The animal may be sacrificed, and tissue samples may be collected and analyzed.

Methods of Screening

Generation of Bacterial Strains with Enhance Ability to Transport Amino Acids

Due to their ease of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Most commonly, this is applied to increase utilization of carbon/energy sources or adapting a strain to environmental stresses (e.g., temperature, pH), whereby mutant strains more capable of growth on the carbon substrate or under stress will outcompete the less adapted strains in the population and will eventually come to dominate the population.

This same process can be extended to any essential metabolite by creating an auxotroph. An auxotroph is a strain incapable of synthesizing an essential metabolite and must therefore have the metabolite provided in the media to grow. In this scenario, by making an auxotroph and passaging it on decreasing amounts of the metabolite, the resulting dominant strains should be more capable of obtaining and incorporating this essential metabolite.

For example, if the biosynthetic pathway for producing an amino acid is disrupted a strain capable of high-affinity capture of said amino acid can be evolved via ALE. First, the strain is grown in varying concentrations of the auxotrophic amino acid, until a minimum concentration to support growth is established. The strain is then passaged at that concentration, and diluted into lowering concentrations of the amino acid at regular intervals. Over time, cells that are most competitive for the amino acid—at growth-limiting concentrations—will come to dominate the population. These strains will likely have mutations in their amino acid-transporters resulting in increased ability to import the essential and limiting amino acid.

Similarly, by using an auxotroph that cannot use an upstream metabolite to form an amino acid, a strain can be evolved that not only can more efficiently import the upstream metabolite, but also convert the metabolite into the essential downstream metabolite. These strains will also evolve mutations to increase import of the upstream metabolite, but may also contain mutations which increase expression or reaction kinetics of downstream enzymes, or that reduce competitive substrate utilization pathways.

A metabolite innate to the microbe can be made essential via mutational auxotrophy and selection applied with growth-limiting supplementation of the endogenous metabolite. However, phenotypes capable of consuming non-native compounds can be evolved by tying their consumption to the production of an essential compound. For example, if a gene from a different organism is isolated which can produce an essential compound or a precursor to an essential compound this gene can be recombinantly introduced and expressed in the heterologous host. This new host strain will now have the ability to synthesize an essential nutrient from a previously non-metabolizable substrate.

Hereby, a similar ALE process can be applied by creating an auxotroph incapable of converting an immediately downstream metabolite and selecting in growth-limiting amounts of the non-native compound with concurrent expression of the recombinant enzyme. This will result in mutations in the transport of the non-native substrate, expression and activity of the heterologous enzyme and expression and activity of downstream native enzymes. It should be emphasized that the key requirement in this process is the ability to tether the consumption of the non-native metabolite to the production of a metabolite essential to growth.

Once the basis of the selection mechanism is established and minimum levels of supplementation have been established, the actual ALE experimentation can proceed. Throughout this process several parameters must be vigilantly monitored. It is important that the cultures are maintained in an exponential growth phase and not allowed to reach saturation/stationary phase. This means that growth rates must be check during each passaging and subsequent dilutions adjusted accordingly. If growth rate improves to such a degree that dilutions become large, then the concentration of auxotrophic supplementation should be decreased such that growth rate is slowed, selection pressure is increased and dilutions are not so severe as to heavily bias subpopulations during passaging. In addition, at regular intervals cells should be diluted, grown on solid media and individual clones tested to confirm growth rate phenotypes observed in the ALE cultures.

Predicting when to halt the stop the ALE experiment also requires vigilance. As the success of directing evolution is tied directly to the number of mutations “screened” throughout the experiment and mutations are generally a function of errors during DNA replication, the cumulative cell divisions (CCD) acts as a proxy for total mutants which have been screened. Previous studies have shown that beneficial phenotypes for growth on different carbon sources can be isolated in about 10^{11.2} CCD¹. This rate can be accelerated by the addition of chemical mutagens to the cultures—such as N-methyl-N-nitro-N-nitrosoguanidine (NTG)—which causes increased DNA replication errors. However, when continued passaging leads to marginal or no improvement in growth rate the population has converged to some fitness maximum and the ALE experiment can be halted.

At the conclusion of the ALE experiment, the cells should be diluted, isolated on solid media and assayed for growth phenotypes matching that of the culture flask. Best performers from those selected are then prepped for genomic DNA and sent for whole genome sequencing. Sequencing with reveal mutations occurring around the genome capable of providing improved phenotypes, but will also contain silent mutations (those which provide no benefit but do not detract from desired phenotype). In cultures evolved in the presence of NTG or other chemical mutagen, there will be significantly more silent, background mutations. If satisfied with the best performing strain in its current state, the user can proceed to application with that strain. Otherwise the contributing mutations can be deconvoluted from the evolved strain by reintroducing the mutations to the parent strain by genome engineering techniques. See Lee, D.-H., Feist, A. M., Barrett, C. L. & Palsson, B. O. Cumulative Number of Cell Divisions as a Meaningful Timescale for Adaptive Laboratory Evolution of Escherichia coli. PLoS ONE 6, e26172 (2011).

Similar methods can be used to generate E. Coli Nissle mutants that consume or import branched chain amino acids, e.g., leucne, valine, and/or isoleucine.

Specific Screen to Identify Strains with Improved BCAA Degradation Enzyme Activity

Screens using genetic selection are conducted to improve BCAA consumption in the genetically engineered bacteria. Toxic BCAA analogs exert their mechanism of action (MOA) by being incorporated into cellular protein, causing cell death. These compounds, e.g., fluoro-leucine and/or aza-leucine, have utility in an untargeted approach to select BCAA enzymes with increased activity. Assuming that these toxic compounds can be metabolized by BCAA enzymes into a non-toxic metabolite, rather than being incorporated into cellular protein, genetically engineered bacteria which have improved BCAA degradation activity can tolerate higher levels of these compounds, and can be screened for and selected on this basis.

Use of Valine and Leucine Sensitivity to Identify Strains with Improved BCAA Degradation Enzyme Activity

Valine and Leucine sensitivity can be used as a genetic screening tool using the E. coli K12 strain. As shown in FIG. 46, There are three AHAS (acetohydroxybutanoate synthase) isozymes in E. coli (AHAS I: ilvBN, AHAS II: ilvGM, and AHAS III: ilvIH). Valine and leucine exert feedback inhibition on AHAS I and AHAS III; AHAS II is resistant to Val and Leu inhibition. E. coli K12 has a frameshift mutation in ilvG (AHAS II) and is unable to produce BCAA endogenously in the presence of valine and leucine. In contrast, E. coli Nissle has a functional ilvG and is insensitive to valine and leucine and therefore cannot be used for this screen. A genetically engineered strain derived from E. coli K12, which more efficiently degrades leucine, has a greater reduction in sensitivity to leucine (through relieving the feedback inhibition on AHAS I and III). As a result, this pathway can be used as a tool to select and identify a strain with improved resistance to leucine.

Use of Leucine Auxotrophy and D-Leucine as a Method to Identify Strains with Improved BCAA Uptake Ability.

Bacterial mutants with increased leucine transport into the bacterial cell may be identified using a leucine auxotroph and providing D-leucine instead of L-leucine in the media, as D-leucine can be imported throught the same transporters. The basis of this strategy is outlined in FIG. 51. The bacteria can grow in the presence of D-leucine, because the bacterial stain has a racemase, which can convert D-leucine to L-leucine. However, the uptake of D-leucine through LivKHMGF is less efficient than the uptake of L-leucine. The leucine auxotroph can still grow if high concentrations of D-Leucine are provided, even though the D-leucine uptake is less efficient than L-leucine uptake. When concentrations of D-leucine in the media are lowered, the cells can no longer grow, unless transport efficiency is increased, ergo, mutants with increased D-leucine uptake can be selected.

Methods of Treatment

Further disclosed herein are methods of treating a disease or disorder associated with the catabolism of a branched chain amino acid. In some embodiments, disclosed herein are methods for reducing, ameliorating, or eliminating one or more symptom(s) associated with these diseases or disorders. In one embodiment, the disorder involving the catabolism of a branched chain amino acid is a metabolic disorder involving the abnormal catabolism of a branched chain amino acid. Metabolic diseases associated with abnormal catabolism of a branched chain amino acid include maple syrup urine disease (MSUD), isovaleric acidemia, propionic acidemia, methylmalonic acidemia, diabetes ketoacidosis, MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria.

In one embodiment, the disease associated with abnormal catabolism of a branched chain amino acid is isovaleric acidemia. In one embodiment, the disease associated with abnormal catabolism of a branched chain amino acid is propionic acidemia. In one embodiment, the disease associated with abnormal catabolism of a branched chain amino acid is methylmalonic acidemia. In another embodiment, the disease associated with abnormal catabolism of a branched chain amino acid is diabetes.

In one embodiment, the disease is maple syrup urine disease (MSUD). Maple syrup urine disease (MSUD), also known as branched-chain ketoaciduria, is an autosomal recessive metabolic disorder caused by impaired activity of the branched-chain α-keto acid dehydrogenase (BCKDH) complex (Skvorak 2009). The overall incidence for MSUD is 1:185,000, although it is higher in certain populations, such as Mennonites, where the incidence is 1:176. The BCKDH complex is responsible for the oxidative decarboxylation of branched-chain keto acids (BCKAs) derived from branched chain amino acids (BCAAs) (Homanics et al. 2006). Patients with MSUD are unable to properly process BCKAs, which can lead to the toxic accumulation of BCAAs and their derivatives in the blood, cerebrospinal fluid and tissues (Skvorak 2009). Specifically, deficiencies of the BCKDH complex in MSUD patients results in accumulation of the BCAAs isoleucine, leucine, and valine, as well as their corresponding branched-chain α-keto acids (BCKAs) α-keto-β-methylvalerate, α-ketoisocaproate, and α-ketoisovalerate) in the tissues in plasma. Clinical manifestations of the disease vary depending on the degree of enzyme deficiency and include poor feeding, vomiting, dehydration, lethargy, hypotonia, seizures, hypoglycemia, ketoacidosis, pancreatitis, coma, and neurological decline (Homanics et al. 2009).

The BCKDH complex is composed of three catalytic components: a dehydrogenase/decarboxylase (E1), which is a heterotetramer composed of two E1α and two E1β subunits, a dihydrolipoyl transacylase (E2), and a dihydrolipoamide dehydrogenase (E3) (Skvorak 2009). Additionally, the complex is associated with two regulatory enzymes, a BCKDH kinase and a BCKDH phosphatase, which control its activity through reversible phosphorylation-dephosphorylation of E1α (Chuang 1998, Homanics et al. 2006). To date, MSUD has been associated with mutations in the E1, E2 and E3 subunits of the BCKDH complex (Cheung 1998, Homanics et al. 2006).

MSUD is a very complex, genetically heterogeneous disease. At least 150 mutations in genes encoding BCKDH complex components have been identified that result in MSUD (Skvorak 2009). For example, see Table C below, adapted from Chuang, J. Pediatrics, 132(3), Part 2, S17-S23, 1998. As indicated below, E2 mutants are the most prevalent in human disease.

TABLE C
MSUD Phenotypes

| | | | Number of |
|---|---|---|---|
| Molecular | Affected | | Mutations |
| Phenotype | Gene | Clinical Phenotype | Identified |
| IA | E1α | Classic, Intermediate MSUD | 15 |
| IB | E1β | Classic MSUD | 4 |
| II | E2 | Classic, thiamine-responsive | 26 |
| | | MSUD | |
| III | E3 | E3-deficient | 4 |
| IV | Kinase | None reported | None reported |
| V | Phosphatase | None reported | None reported |

Currently available treatments for MSUD are inadequate for the long term management of the disease and have severe limitations (Svkvorak 2009). A low protein/BCAA-restricted diet, with micronutrient and vitamin supplementation, as necessary, is the widely accepted long-term disease management strategy for MSUD (Homanics et al. 2006). However, BCAA-intake restrictions can be particularly problematic since BCAAs can only be acquired through diet and are necessary for several metabolic activities (Skvorak 2009). Even with proper monitoring and patient compliance, BCAA dietary restrictions result in a high incidence of mental retardation and mortality (Skvorak 2009, Homanics et al 2009). Further, a few cases of MSUD have been treated by liver transplantation (Popescu and Dima 2012) or treatment with phenylbutyrate. However, the limited availability of donor organs, the costs associated with the transplantation itself, and the undesirable effects associated with continued immunosuppressant therapy limit the practicality of liver transplantation for treatment of disease (Homanics et al. 2012, Popescu and Dima 2012). Therefore, there is significant unmet need for effective, reliable, and/or long-term treatment for MSUD.

The present disclosure surprisingly demonstrates that pharmaceutical compositions comprising the recombinant bacterial cells disclosed herein may be used to treat metabolic diseases involving the abnormal catabolism of a branched chain amino acid, such as MSUD. In one embodiment, the metabolic disease is selected from the group consisting of classic MSUD, intermediate MSUD, intermittent MSUD, E3-Deficient MSUD, and thiamine-responsive MSUD. In one embodiment, the disease is classic MSUD. In another embodiment, the disease is intermediate MSUD. In another embodiment, the disease is intermittent MSUD. In another embodiment, the disease is E3-deficient MSUD. In another embodiment, the disease is thiamine-responsive MSUD.

In one embodiment, the subject having MSUD has a mutation in an E1α gene. In another embodiment, the subject having MSUD has a mutation in the E1β gene. In another embodiment, the subject having MSUD has a mutation in the E2 gene. In another embodiment, the subject having MSUD has a mutation in the E3 gene.

In one embodiment, the target degradation rates of branched chain amino acids from food intake in breastfed infants and adults is as indicated in the Table 13, below.

TABLE 13
Target Degradation rates for BCAA.

| | Age (year) | | | | | | | | |
|---|---|---|---|---|---|---|---|---|---|
| | <1 | | | 1-3 | | | 4-8 | | |
| | Amino acid: Leu (L) Val (V) Ile (I) | | | | | | | | |
| | L | V | I | L | V | I | L | V | I |
| MSUD patient | 40 | 30 | 20 | 20 | 10 | 5 | 5 | 10 | 5 |
| daily tolerance | | | | | | | | | |
| (mg/kg) | | | | | | | | | |
| Recommended | 93 | 58 | 43 | 63 | 37 | 28 | 49 | 28 | 22 |
| Dietary | | | | | | | | | |
| Allowance | | | | | | | | | |
| (RDA) (mg/kg) | | | | | | | | | |
| Target | 53 | 28 | 23 | 43 | 27 | 23 | 44 | 18 | 17 |
| reduction | | | | | | | | | |
| (mg/kg) | | | | | | | | | |
| Target | 530 | 280 | 230 | 602 | 378 | 322 | 1144 | 468 | 442 |
| reduction (mg); | | | | | | | | | |
| (based on 10, | | | | | | | | | |
| 14, 26, 41 and | | | | | | | | | |
| 70 kg weight for | | | | | | | | | |
| the different | | | | | | | | | |
| age groups) | | | | | | | | | |
| Target | 4.04 | 2.39 | 1.75 | 4.59 | 3.23 | 2.45 | 8.72 | 3.99 | 3.37 |
| reduction | | | | | | | | | |
| (mmol) | | | | | | | | | |
| Target | 0.56 | 0.33 | 0.24 | 0.64 | 0.45 | 0.34 | 1.21 | 0.55 | 0.47 |
| degradation | | | | | | | | | |
| rate (μmol/10⁹ | | | | | | | | | |
| CFUs/hr); | | | | | | | | | |
| (based on | | | | | | | | | |
| 3.10¹¹ | | | | | | | | | |
| CFUs/day | | | | | | | | | |
| dose) | | | | | | | | | |

| | | | |
|---|---|---|---|
| Combined | 1.14 | 1.43 | 2.23 |
| BCAA target | | | |
| degradation | | | |
| rate (μmol/10⁹ | | | |
| CFUs/hr) | | | |

Age (year)

| | |
|---|---|
| 8-12 | >12 |

Amino acid: Leu (L) Val (V) Ile (I)

| | L | V | I | L | V | I |
|---|---|---|---|---|---|---|
| MSUD patient | 5 | 10 | 5 | 5 | 10 | 5 |
| daily tolerance | | | | | | |
| (mg/kg) | | | | | | |
| Recommended | 46 | 26 | 20 | 46 | 26 | 20 |
| Dietary | | | | | | |
| Allowance | | | | | | |
| (RDA) (mg/kg) | | | | | | |
| Target | 41 | 16 | 15 | 41 | 16 | 15 |
| reduction | | | | | | |
| (mg/kg) | | | | | | |
| Target | 1681 | 656 | 615 | 2870 | 1120 | 1050 |
| reduction (mg); | | | | | | |
| (based on 10, | | | | | | |
| 14, 26, 41 and | | | | | | |
| 70 kg weight for | | | | | | |
| the different | | | | | | |
| age groups) | | | | | | |
| Target | 12.81 | 5.60 | 4.69 | 21.88 | 9.56 | 8.00 |
| reduction | | | | | | |
| (mmol) | | | | | | |
| Target | 1.78 | 0.78 | 0.65 | 3.04 | 1.33 | 1.11 |
| degradation | | | | | | |
| rate (μmol/10⁹ | | | | | | |
| CFUs/hr); | | | | | | |
| (based on | | | | | | |
| 3.10¹¹ | | | | | | |
| CFUs/day | | | | | | |
| dose) | | | | | | |

| | | |
|---|---|---|
| Combined | 3.21 | 5.48 |
| BCAA target | | |
| degradation | | |
| rate (μmol/10⁹ | | |
| CFUs/hr) | | |

In one embodiment, the target degradation rates of branched chain amino acids from food intake in breastfed infants and adults is as indicated in the charts, below.

The leucine consumption kinetics and dosing are set forth in Table G. Food intake is based on adult recommended daily allowance of 40 mg/kg/day. MSUD patients are primarily children with restricted protein intake.

In another embodiment, the disorder involving the catabolism of a branched chain amino acid is a disorder caused by the activation of mTor (mammalian target of rapamycin). mTor is a serine-threonine kinase and has been implicated in a wide range of biological processes including transcription, translation, autophagy, actin organization and ribosome biogenesis, cell growth, cell proliferation, cell motility, and survival. mTOR exists in two complexes, mTORC1 and mTORC2. mTORC1 contains the raptor subunit and mTORC2 contains rictor. These complexes are differentially regulated, and have distinct substrate specificities and rapamycin sensitivity. For example, mTORC1 phosphorylates S6 kinase (S6K) and 4EBP1, promoting increased translation and ribosome biogenesis to facilitate cell growth and cell cycle progression. S6K also acts in a feedback pathway to attenuate PI3K/Akt activation. mTORC2 is generally insensitive to rapamycin and is thought to modulate growth factor signaling by phosphorylating the C-terminal hydrophobic motif of some AGC kinases, such as Akt.

It is known in the art that mTor activation is caused by branched chain amino acids or alpha keto acids in the subject (see, for example, Harlan et al., Cell Metabolism, 17:599-606, 2013). Specifically, activation of mTorC1 (mTor complex 1) is caused by leucine (see Han et al., Cell, 149:410-424, 2012 and Lynch, J. Nutr., 131(31:861S-865S, 2001). Thus, in one embodiment, the disclosure provides methods of treating disorders involving the catabolism of leucine, caused by the activation of mTor by leucine in the subject. In one embodiment, the leucine levels in the subject are normal, and lowering leucine levels in the subject leads to the decreased activity of mTor and, thus, treatment of the disease. In another embodiment, the leucine levels in the subject are increased, and lowering leucine levels in the subject leads to the decreased activity of mTor and, thus, treatment of the disease. In one embodiment, the activation of mTor is increased as compared to the normal level of activation of mTor in a healthy subject, and lowering leucine levels in the subject leads to the decreased activation of mTor and, thus, treatment of the disease. In one embodiment, the level of activity of mTor is increased as compared to the normal level of activity of mTor in a healthy subject, and lowering leucine levels in the subject leads to the decreased activity of mTor and, thus, treatment of the disease. In another embodiment, the expression of mTor is increased as compared to the normal level of expression of mTor in a healthy subject, and lowering leucine levels in the subject leads to the decreased activity of mTor and, thus, treatment of the disease. In one embodiment, the activation of mTor is an abnormal activation of mTor.

Diseases caused by the activation of mTor are known in the art. See, for example, Laplante and Sabatini, Cell, 149(2):74-293, 2012. As used herein, the term “disease caused by the activation of mTor” includes cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia.

In another aspect, the disclosure provides methods for decreasing the plasma level of at least one branched chain amino acid or branched chain α-keto acid in a subject by administering a pharmaceutical composition comprising a bacterial cell disclosed herein to the subject, thereby decreasing the plasma level of the at least one branched chain amino acid or branched chain alpha-keto acid or other BCAA metabolite in the subject. In one embodiment, the subject has a disease or disorder involving the catabolism of a branched chain amino acid. In one embodiment, the disorder involving the catabolism of a branched chain amino acid is a metabolic disorder involving the abnormal catabolism of a branched chain amino acid. In another embodiment, the disorder involving the catabolism of a branched chain amino acid is a disorder caused by the activation of mTor. In one embodiment, the disease or disorder is a maple syrup urine disorder (MSUD). In one embodiment, the branched chain amino acid is leucine, isoleucine, or valine. In one embodiment, the branched chain amino acid is leucine. In another embodiment, the branched chain amino acid is isoleucine. In another embodiment, the branched chain amino acid is valine. In another embodiment, the branched chain α-keto acid is α-ketoisocaproic acid (KIC). In another embodiment, the branched chain α-keto acid is α-ketoisovaleric acid (KIV). In another embodiment, the branched chain α-keto acid is α-keto-β-methylvaleric acid (KMV). In other embodiments, the BCAA metabolite to be decrease is selected from any of BCAA metabolies shown in FIG. 1.

In some embodiments, the disclosure provides methods for reducing, ameliorating, or eliminating one or more symptom(s) associated with these diseases, including but not limited to neurological deficits, mental retardation, brain damage, brain oedema, blindness, branched chain α-keto acid acidosis, myelinization failure, hyperammonaemia, coma, developmental delay, neurological impairment, failure to thrive, ketoacidosis, seizure, ataxia, neurodegeneration, hypotonia, lactic acidosis, recurrent myoglobinuria, and/or liver failure. In some embodiments, the disease is secondary to other conditions, e.g., liver disease.

In certain embodiments, the bacterial cells disclosed herein are capable of catabolizing branched chain amino acid(s), e.g., leucine, in the diet of the subject in order to treat a disease associated with catabolism of a branched chain amino acid, e.g., MSUD. In these embodiments, the bacterial cells are delivered simultaneously with dietary protein. In another embodiment, the bacterial cells are delivered simultaneously with phenylbutyrate. In another embodiment, the bacterial cells are delivered simultaneously with a thiamine supplement. In some embodiments, the bacterial cells and dietary protein are delivered after a period of fasting or leucine-restricted dieting. In these embodiments, a patient suffering from a disorder involving the catabolism of a branched chain amino acid, e.g., MSUD, may be able to resume a substantially normal diet, or a diet that is less restrictive than a leucine-free diet. In some embodiments, the bacterial cells may be capable of catabolizing leucine from additional sources, e.g., the blood, in order to treat a disease associated with the catabolism of a branched chain amino acid, e.g., MSUD. In these embodiments, the bacterial cells need not be delivered simultaneously with dietary protein, and a leucine gradient is generated, e.g., from blood to gut, and the recombinant bacteria catabolize the branched chain amino acid, e.g., leucine, and reduce plasma levels of the branched chain amino acid, e.g., leucine.

The method may comprise preparing a pharmaceutical composition with at least one genetically engineered species, strain, or subtype of bacteria described herein, and administering the pharmaceutical composition to a subject in a therapeutically effective amount. In some embodiments, the bacterial cells disclosed herein are administered orally, e.g., in a liquid suspension. In some embodiments, the bacterial cells disclosed herein are lyophilized in a gel cap and administered orally. In some embodiments, the bacterial cells disclosed herein are administered via a feeding tube or gastric shunt. In some embodiments, the bacterial cells disclosed herein are administered rectally, e.g., by enema. In some embodiments, the genetically engineered bacteria are administered topically, intraintestinally, intrajejunally, intraduodenally, intraileally, and/or intracolically.

In certain embodiments, the administering the pharmaceutical composition described herein reduces branched chain amino acid levels in a subject. In some embodiments, the methods of the present disclosure reduce the branched chain amino acid levels, e.g., leucine levels, in a subject by at least about 10%, 20%, 25%, 30%, 40%, 50%, 60%, 70%, 75%, 80%, 85%, 90%, 95%, or more. In another embodiment, the methods of the present disclosure reduce the branched chain amino acid levels, e.g., leucine levels, in a subject by at least two-fold, three-fold, four-fold, five-fold, six-fold, seven-fold, eight-fold, nine-fold, or ten-fold. In some embodiments, reduction is measured by comparing the branched chain amino acid level in a subject before and after administration of the pharmaceutical composition. In one embodiment, the branched chain amino acid level is reduced in the gut of the subject. In another embodiment, the branched chain amino acid level is reduced in the blood of the subject. In another embodiment, the branched chain amino acid level is reduced in the plasma of the subject. In another embodiment, the branched chain amino acid level is reduced in the brain of the subject.

In one embodiment, the pharmaceutical composition described herein is administered to reduce branched chain amino acid levels in a subject to normal levels. In another embodiment, the pharmaceutical composition described herein is administered to reduce branched chain amino acid levels in a subject below normal levels to, for example, decrease the activation of mTor.

In certain embodiments, the pharmaceutical composition described herein is administered to reduce branched chain α-keto-acid levels in a subject. In some embodiments, the methods of the present disclosure reduce the branched chain α-keto-acid levels, in a subject by at least about 10%, 20%, 25%, 30%, 40%, 50%, 60%, 70%, 75%, 80%, 85%, 90%, 95%, or more as compared to levels in an untreated or control subject. In another embodiment, the methods of the present disclosure reduce the branched chain α-keto-acid levels, in a subject by at least two-fold, three-fold, four-fold, five-fold, six-fold, seven-fold, eight-fold, nine-fold, or ten-fold. In some embodiments, reduction is measured by comparing the branched chain α-keto-acid levels in a subject before and after administration of the pharmaceutical composition. In one embodiment, the branched chain α-keto-acid level is reduced in the gut of the subject. In another embodiment, the branched chain α-keto-acid level is reduced in the blood of the subject. In another embodiment, the branched chain α-keto-acid level is reduced in the plasma of the subject. In another embodiment, the branched chain α-keto-acid level is reduced in the brain of the subject.

In one embodiment, the pharmaceutical composition described herein is administered to reduce the branched chain α-keto-acid level in a subject to a normal level. In another embodiment, the pharmaceutical composition described herein is administered to reduce the branched chain α-keto-acid level in a subject below a normal level to, for example, decrease the activation of mTor.

In some embodiments, the method of treating the disorder involving the catabolism of a branched chain amino acid, e.g., MSUD, allows one or more symptoms of the condition or disorder to improve by at least about 10%, 20%, 30%, 40%, 50%, 60%, 70%, 80%, 90%, 95%, or more. In some embodiments, the method of treating the disorder involving the catabolism of a branched chain amino acid, e.g., MSUD, allows one or more symptoms of the condition or disorder to improve by at least about two-fold, three-fold, four-fold, five-fold, six-fold, seven-fold, eight-fold, nine-fold, or ten-fold.

Before, during, and after the administration of the pharmaceutical composition, branched chain amino acid levels, e.g., leucine levels, in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions disclosed herein to reduce levels of the branched chain amino acid, e.g., leucine. In some embodiments, the methods may include administration of the compositions disclosed herein to reduce the branched chain amino acid, e.g., leucine, to undetectable levels in a subject. In some embodiments, the methods may include administration of the compositions disclosed herein to reduce the branched chain amino acid, e.g., leucine, concentrations to undetectable levels, or to less than about 1%, 2%, 5%, 10%, 20%, 25%, 30%, 40%, 50%, 60%, 70%, 75%, 80%, 85%, 90%, or 95% of the subject's branched chain amino acid levels prior to treatment.

In some embodiments, the recombinant bacterial cells disclosed herein produce a branched chain amino acid catabolism enzyme, e.g., KivD, BCKD and/or other BCAA catabolism enzyme, BCAA transporter, BCAA binding protein, etc, under exogenous environmental conditions, such as the low-oxygen environment of the mammalian gut, to reduce levels of branched chain amino acids in the blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

In one embodiment, the bacteria disclosed herein reduce plasma levels of the branched chain amino acid, e.g., leucine, will be reduced to less than 4 mg/dL. In one embodiment, the bacteria disclosed herein reduce plasma levels of the branched chain amino acid, e.g., leucine, will be reduced to less than 3.9 mg/dL. In one embodiment, the bacteria disclosed herein reduce plasma levels of the branched chain amino acid, e.g., leucine, will be reduced to less than 3.8 mg/dL, 3.7 mg/dL, 3.6 mg/dL, 3.5 mg/dL, 3.4 mg/dL, 3.3 mg/dL, 3.2 mg/dL, 3.1 mg/dL, 3.0 mg/dL, 2.9 mg/dL, 2.8 mg/dL, 2.7 mg/dL, 2.6 mg/dL, 2.5 mg/dL, 2.0 mg/dL, 1.75 mg/dL, 1.5 mg/dL, 1.0 mg/dL, or 0.5 mg/dL.

In one embodiment, the subject has plasma levels of at least 4 mg/dL prior to administration of the pharmaceutical composition disclosed herein. In another embodiment, the subject has plasma levels of at least 4.1 mg/dL, 4.2 mg/dL, 4.3 mg/dL, 4.4 mg/dL, 4.5 mg/dL, 4.75 mg/dL, 5.0 mg/dL, 5.5 mg/dL, 6 mg/dL, 7 mg/dL, 8 mg/dL, 9 mg/dL, or 10 mg/dL prior to administration of the pharmaceutical composition disclosed herein.

Certain unmodified bacteria will not have appreciable levels of branched chain amino acid, e.g., leucine, processing. In embodiments using genetically modified forms of these bacteria, processing of branched chain amino acids, e.g., leucine, will be appreciable under exogenous environmental conditions.

Branched chain amino acid levels, e.g., leucine levels, may be measured by methods known in the art, e.g., blood sampling and mass spectrometry. In some embodiments, branched chain amino acid catabolism enzyme expression is measured by methods known in the art. In another embodiment, branched chain amino acid catabolism enzyme activity is measured by methods known in the art to assess BCAA activity.

In certain embodiments, the recombinant bacteria are E. coli Nissle. The recombinant bacteria may be destroyed, e.g., by defense factors in the gut or blood serum (Sonnenborn et al., 2009) or by activation of a kill switch, several hours or days after administration. Thus, the pharmaceutical composition comprising the recombinant bacteria may be re-administered at a therapeutically effective dose and frequency. In alternate embodiments, the recombinant bacteria are not destroyed within hours or days after administration and may propagate and colonize the gut.

In one embodiments, the bacterial cells disclosed herein are administered to a subject once daily. In another embodiment, the bacterial cells disclosed herein are administered to a subject twice daily. In another embodiment, the bacterial cells disclosed herein are administered to a subject in combination with a meal. In another embodiment, the bacterial cells disclosed herein are administered to a subject prior to a meal. In another embodiment, the bacterial cells disclosed herein are administered to a subject after a meal. The dosage of the pharmaceutical composition and the frequency of administration may be selected based on the severity of the symptoms and the progression of the disease. The appropriate therapeutically effective dose and/or frequency of administration can be selected by a treating clinician.

The methods disclosed herein may comprise administration of a composition disclosed herein alone or in combination with one or more additional therapies, e.g., the phenylbutyrate, thiamine supplementation, and/or a low-branched chain amino acid, e.g., a low-leucine, diet. An important consideration in the selection of the one or more additional therapeutic agents is that the agent(s) should be compatible with the bacteria disclosed herein, e.g., the agent(s) must not interfere with or kill the bacteria. In some embodiments, the genetically engineered bacteria are administered in combination with a low protein diet. In some embodiments, administration of the genetically engineered bacteria provides increased tolerance, sothat the patient can consume more protein.

The methods disclosed herein may further comprise isolating a plasma sample from the subject prior to administration of a composition disclosed herein and determining the level of the branched chain amino acid, e.g., leucine, or branched chain alpha-keto-acid in the sample. In some embodiments, the methods disclosed herein may further comprise isolating a plasma sample from the subject after to administration of a composition disclosed herein and determining the level of the branched chain amino acid, e.g., leucine, or branched chain alpha-keto-acid in the sample.

In one embodiment, the methods disclosed herein further comprise comparing the level of the branched chain amino acid or branched chain α-keto-acid in the plasma sample from the subject after administration of a composition disclosed herein to the subject to the plasma sample from the subject before administration of a composition disclosed herein to the subject. In one embodiment, a reduced level of the branched chain amino acid or branched chain alpha-keto-acid in the plasma sample from the subject after administration of a composition disclosed herein indicates that the plasma levels of the branched chain amino acid or branched chain alpha-keto-acid are decreased, thereby treating the disorder involving the catabolism of the branched chain amino acid in the subject. In one embodiment, the plasma level of the branched chain amino acid or branched chain α-keto-acid is decreased at least 10%, 20%, 30%, 40, 50%, 60%, 70%, 80%, 90%, or 100% in the sample after administration of the pharmaceutical composition as compared to the plasma level in the sample before administration of the pharmaceutical composition. In another embodiment, the plasma level of the branched chain amino acid or branched chain α-keto-acid is decreased at least two-fold, three-fold, four-fold, or five-fold in the sample after administration of the pharmaceutical composition as compared to the plasma level in the sample before administration of the pharmaceutical composition.

In one embodiment, the methods disclosed herein further comprise comparing the level of the branched chain amino acid or branched chain α-keto-acid in the plasma sample from the subject after administration of a composition disclosed herein to a control level of the branched chain amino acid or branched chain alpha-keto-acid. In one embodiment, the control level of the branched chain amino acid is 4 mg/dL. In one embodiment, the subject is considered treated if the level of branched chain amino acid, e.g., leucine, in the plasma sample from the subject after administration of the pharmaceutical composition disclosed herein, is less than 4 mg/dL. In one embodiment, the subject is considered treated if the level of branched chain amino acid, e.g., leucine, in the plasma sample from the subject after administration of the pharmaceutical composition disclosed herein is less than 3.9, 3.8, 3.7, 3.6, 3.5, 3.4, 3.3, 3.2, 3.1, 3.0, 2.5, 2.0, 1.5, 1.0 or 0.5 mg/dL.

### Examples

The present disclosure is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Development of Recombinant Bacterial Cells

### Example 1. Construction of Plasmids Encoding Branched Chain Amino Acid

Importers and Branched Chain Amino Acid Catabolism Enzyme

The kivD gene of lactococcus lactis IFPL730 (sequence: SEQ ID NO: 1) was synthesized (Genewiz), fused to the Tet promoter, cloned into the high-copy plasmid pUC57-Kan by Gibson assembly (SEQ ID NO: 2), and transformed into E. coli DH5α as described in Example 3 to generate the plasmid pTet-kivD. The bkd operon of Pseudomonas aeruginosa PAO1 fused to the Tet promoter (SEQ ID NO:3) was synthesized (Genewiz) and cloned into the high-copy plasmid pUC57-Kan to generate the plasmid pTet-bkd. The bkd operon of Pseudomonas aeruginosa PAO1 fused to the leuDH gene from PA01 and the Tet promoter (SEQ ID NO: 4) was synthesized (Genewiz) and cloned into the high-copy plasmid pUC57-Kan to generate the plasmid pTet-leuDH-bkd. The livKHMGF operon from E. coli Nissle fused to the Tet promoter (SEQ ID NO:5) was synthesized (Genewiz), cloned into the pKIKO-lacZ plasmid (SEQ ID NO:6) by Gibson assembly and transformed into E. coli PIR1 as described in Example 3 to generate the pTet-livKHMGF (SEQ ID NO:7).

### Example 2. Generation of Recombinant Bacterial Comprising a Genetic

Modification that Reduces Export of a Branched Chain Amino Acid

E. coli Nissle was transformed with the pKD46 plasmid encoding the lambda red proteins under the control of an arabinose-inducible promoter as follows. An overnight culture of E. coli Nissle grown at 37° C. was diluted 1:100 in 4 mL of lysogeny broth (LB) and grown at 37° C. until it reached an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pKD46 miniprep was added to the cells, mixed by pipetting, and pipetted into a sterile, chilled 1 mm cuvette. The cuvette was placed into the sample chamber, and the electric pulse was applied. 500 uL of room-temperature SOC media was immediately added, and the mixture was transferred to a culture tube and incubated at 30° C. for 1 hr. The cells were spread out on an LB plate containing 100 ug/mL carbenicillin and incubated at 30° C.

A ΔleuE deletion construct with 77 bp and a 100 bp flanking leuE homology regions and a kanamycin resistant cassette flanked by FRT recombination site (SEQ ID NO: 6) was generated by PCR, column-purified and transformed into E. coli Nissle pKD46 as follows. An overnight culture of E. coli Nissle pKD46 grown in 100 ug/mL carbenicillin at 30° C. was diluted 1:100 in 5 mL of LB supplemented with 100 ug/mL carbenicillin, 0.15% arabinose and grown until it reaches an OD₆₀₀ of 0.4-0.6. The bacteria were aliquoted equally in five 1.5 mL microcentrifuge tubes, centrifuged at 13,000 rpm for 1 min and the supernatant was removed. The cells were then washed three times in pre-chilled 10% glycerol and combined in 50 uL pre-chilled 10% glycerol. The electroporator was set to 1.8 kV. 2 uL of a the purified ΔleuE deletion PCR fragment are then added to the cells, mixed by pipetting, and pipetted into a sterile, chilled 1 mm cuvette. The cuvette was placed into the sample chamber, and the electric pulse was applied. 500 uL of room-temperature SOC media was immediately added, and the mixture was transferred to a culture tube and incubated at 37° C. for 1 hr. The cells were spread out on an LB plate containing 50 ug/mL kanamycin. Five kanamycin-resistant transformants were then checked by colony PCR for the deletion of the leuE locus.

The kanamycin cassette was then excised from the ΔleuE deletion strain as follows. ΔleuE was transformed with the pCP20 plasmid encoding the Flp recombinase gene. An overnight culture of ΔleuE grown at 37° C. in LB with 50 ug/mL kanamycin was diluted 1:100 in 4 mL of LB and grown at 37° C. until it reaches an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pCP20 miniprep was added to the cells, mixed by pipetting, and pipetted into a sterile, chilled 1 mm cuvette. The dry cuvette was placed into the sample chamber, and the electric pulse was applied. 500 uL of room-temperature SOC media was immediately added, and the mixture was transferred to a culture tube and incubated at 30° C. for 1 hr. The cells were spread out on an LB plate containing 100 ug/mL carbenicillin and incubated at 30° C. Eight transformants were then streaked on an LB plate and were incubated overnight at 43° C. One colony per transformant was picked and resuspended in 10 uL LB and 3 uL of the suspension were pipetted on LB, LB with 50 ug/mL Kanamycin or LB with 100 ug/mL carbenicillin. The LB and LB Kanamycin plates were incubated at 37° C. and the LB Carbenicillin plate was incubated at 30° C. Colonies showing growth on LB alone were selected and checked by PCR for the excision of the Kanamycin cassette.

### Example 3. Generation of Recombinant Bacteria Comprising a Transporter of a Branched Chain Amino Acid and/or a Branched Chain Amino Acid Catabolism Enzyme and Lacking an Exporter of a Branched Chain Amino Acid

pTet-kivD, pTet-bkd, pTet-leuDH-bkd and pTet-livKHFGF plasmids described above were transformed into E. coli Nissle (pTet-kivD), Nissle (pTet-kivD, pTet-bkd, pTet-leuDH-bkd), DH5α (pTet-kivD, pTet-bkd, pTet-leuDH-bkd) or PIR1 (pTet-livKHMGF). All tubes, solutions, and cuvettes were pre-chilled to 4° C. An overnight culture of E. coli (Nissle, ΔleuE, DH5α or PIR1) was diluted 1:100 in 4 mL of LB and grown until it reached an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pTet-kivD, pTet-bkd, pTet-leuDH-bkd or pTet-livKHMGF miniprep was added to the cells, mixed by pipetting, and pipetted into a sterile, chilled 1 mm cuvette. The dry cuvette was placed into the sample chamber, and the electric pulse was applied. 500 uL of room-temperature SOC media was immediately added, and the mixture was transferred to a culture tube and incubated at 37° C. for 1 hr. The cells were spread out on an LB plate containing 50 ug/mL Kanamycin for pTet-kivD, pTet-bkd and pTet-leuDH-bkd or 100 ug/mL carbenicillin for pTet-livKHMGF.

### Example 4. Generation of Recombinant Bacteria Comprising a Transporter of a Branched Chain Amino Acid and a Genetic Modification that Reduces Export of a Branched Chain Amino Acid

E. coli Nissle ΔleuE was transformed with the pKD46 plasmid encoding the lambda red proteins under the control of an arabinose-inducible promoter as follows. An overnight culture of E. coli Nissle ΔleuE grown at 37° C. was diluted 1:100 in 4 mL of LB and grown at 37° C. until it reached an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pKD46 miniprep was added to the cells, mixed by pipetting, and pipetted into a sterile, chilled 1 mm cuvette. The dry cuvette was placed into the sample chamber, and the electric pulse was applied. 500 uL of room-temperature SOC media was immediately added, and the mixture was transferred to a culture tube and incubated at 30° C. for 1 hr. The cells were spread out on an LB plate containing 100 ug/mL carbenicillin and incubated at 30° C.

The DNA fragment used to integrate Tet-livKHMGF into E. coli Nissle lacZ (SEQ ID NO: 10, FIG. 31) was amplified by PCR from the pTet-livKHMGF plasmid, column-purified and transformed into ΔleuE pKD46 as follows. An overnight culture of the E. coli Nissle ΔleuE pKD46 strain grown in LB at 30° C. with 100 ug/mL carbenicillin was diluted 1:100 in 5 mL of lysogeny broth (LB) supplemented with 100 ug/mL carbenicillin, 0.15% arabinose and grown at 30° C. until it reached an OD₆₀₀ of 0.4-0.6. The bacteria were aliquoted equally in five 1.5 mL microcentrifuge tubes, centrifuged at 13,000 rpm for 1 min and the supernatant was removed. The cells were then washed three times in pre-chilled 10% glycerol and combined in 50 uL pre-chilled 10% glycerol. The electroporator was set to 1.8 kV. 2 uL of a the purified Tet-livKHMGF PCR fragment were then added to the cells, mixed by pipetting, and pipetted into a sterile, chilled 1 mm cuvette. The dry cuvette was placed into the sample chamber, and the electric pulse was applied. 500 uL of room-temperature SOC media was immediately added, and the mixture was transferred to a culture tube and incubated at 37° C. for 1 hr. The cells were spread out on an LB plate containing 20 ug/mL chloramphenicol, 40 ug/mL X-Gal and incubated overnight at 37° C. White chloramphenicol resistant transformants were then checked by colony PCR for integration of Tet-livKHMGF into the lacZ locus.

### Functional Assays Using Recombinant Bacterial Cells

### Example 5. Functional Assay Demonstrating that the Recombinant Bacterial Cells Disclosed Herein Decrease Branched Chain Amino Acid Concentration

For in vitro studies, all incubations were performed at 37° C. Cultures of E. coli Nissle ΔleuE, ΔleuE+pTet-kivD, ΔleuE+pTet-bkd, ΔleuE+pTet-leuDH-bkd, ΔleuE lacZ:Tet-livKHMGF, ΔleuE lacZ:Tet-livKHMGF+pTet-kivD, ΔleuE lacZ:Tet-livKHMGF+pTet-bkd, ΔleuE lacZ:Tet-livKHMGF+pTet-leuDH-bkd were grown overnight in LB, LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD, Bkd, LeuDH and LivKHFMG, and bacteria were grown for another 3 hours. Bacteria were then pelleted, washed, and resuspended in minimal media, and supplemented with 0.5% glucose and 2 mM leucine. Aliquots were removed at 0 h, 1.5 h, 6 h and 18 h for leucine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in 90 uL 10% acetonitrile, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 95% A, 5% B

0.5 min: 95% A, 5% B

1 min: 10% A, 90% B

2.5 min: 10% A, 90% B

2.51 min: 95% A, 5% B

3.5 min: 95% A, 5% B

The Q1/Q3 transitions used for leucine and L-leucine-5,5,5-d₃ were 132.1/86.2 and 135.1/89.3 respectively.

As shown in FIG. 16, leucine was rapidly degraded by the expression of kivD in the Nissle ΔleuE strain. After 6 h of incubation, leucine concentration dropped by over 99% in the presence of ATC. This effect was even more pronounced in the case of ΔleuE expressing both kivD and the leucine transporter livKHMGF where leucine is undetectable after 6 h of incubation. As shown in FIG. 17, the expression of the bkd complex also leads rapidly to the degradation of leucine. After 6 h of incubation, 99% of leucine was degraded. The expression of the leucine transporter livKHMGF, in parallel with the expression of leuDH and bkd leads to the complete degradation of leucine after 18 h.

### Example 6. Simultaneous Degradation of Branched Chain Amino Acids by Recombinant Bacteria Expressing a Branched Chain Amino Acid Catabolism Enzyme and an Importer of a Branched Chain Amino Acid

In these studies, all incubations were performed at 37° C. Cultures of E. coli Nissle, Nissle+pTet-kivD, ΔleuE+pTet-kivD, ΔleuE lacZ:Tet-livKHMGF+pTet-kivD were grown overnight in LB, LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD and LivKHFMG, and bacteria were grown for another 3 hours. Bacteria were then pelleted, washed, and resuspended in minimal media, and supplemented with 0.5% glucose and the three branched chain amino acids (leucine, isoleucine and valine, 2 mM each). Aliquots were removed at 0 h, 1.5 h, 6 h and 18 h for leucine, isoleucine and valine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

Isoleucine: 132.1/86.2

Valine: 118.1/72

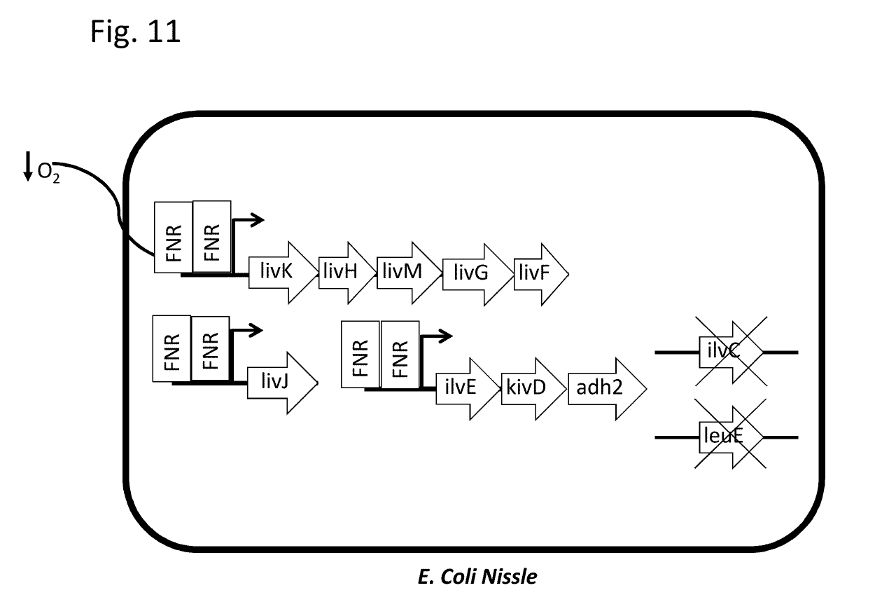

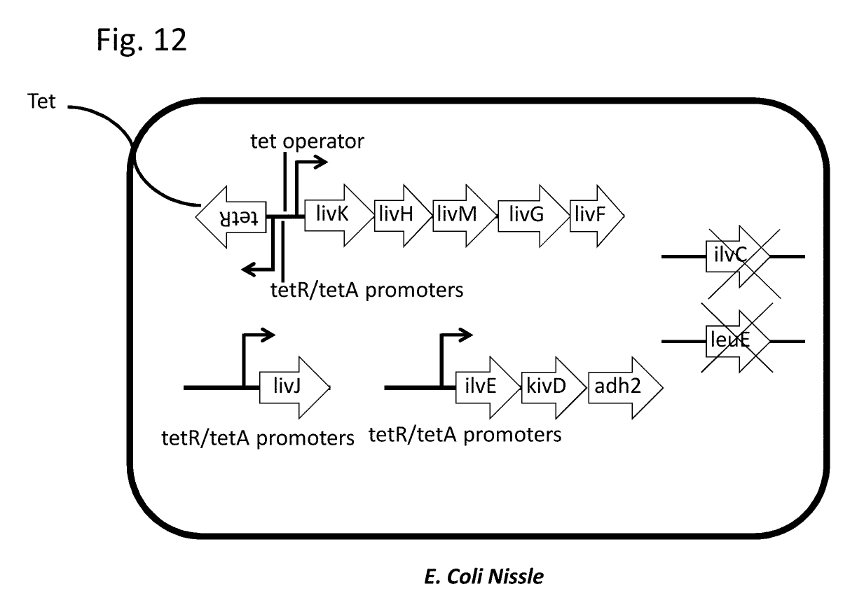

As shown in FIGS. 11A-11C, leucine, isoleucine and valine were all degraded by the expression of kivD in E. coli Nissle. At 18 h, 96.8%, 67.2% and 52.1% of leucine, isoleucine and valine respectively were degraded in Nissle expressing kivD in the presence of ATC. The efficiency of leucine and isoleucine degradation was further improved by expressing kivD in the ΔleuE background strain with a 99.8% leucine and 80.6% isoleucine degradation at 18 h Finally, an additional increase in leucine and isoleucine degradation was achieved by expressing the leucine transporter livKHMGF in the Nissle ΔleuE pTet-kivD strain with a 99.98% leucine and 95.5% isoleucine degradation at 18 h. No significant improvement in valine degradation was observed in the ΔleuE deletion strain expressing livKHMGF.

### Example 7. Degradation of Leucine and its Ketoacid Derivative, Ketoisocaproate (KIC) by Recombinant Bacterial Cells In Vitro

Leucine and its ketoacid derivative, alpha-ketoisocaproate (KIC), are two major metabolites which accumulate to toxic levels in MSUD patients. Different synthetic probiotic E. coli Nissle strains were engineered to degrade leucine and KIC in order to determine the rate of degradation of leucine and KIC in these strains.

All strains were derived from the human probiotic strain E. coli Nissle 1917. A ΔleuE deletion strain (deleted for the leucine exporter leuE) was generated by lambda red-recombination. A copy of the high-affinity leucine ABC transporter livKHMGF under the control of a tetracycline-inducible promoter (Ptet) was inserted into the lacZ locus of the ΔleuE deletion strain by lambda-red recombination. In order to avoid endogenous production of BCAA and KIC, the biosynthetic gene ilvC was deleted in the ΔleuE; lacZ:tetR-Pₜₑₜ-livKHMGF strain by P1 transduction using the ΔilvC BW25113 E. coli strain as donor to generate the SYN469 strain (ΔleuE ΔilvC; lacZ:tetR-Pₜₑₜ-livKHMGF).

The SYN469 strain was then transformed with five different constructs under the control of Ptet on the high-copy plasmid pUC57-Kan (FIG. 23). The components of the constructs were:

the leucine dehydrogenase leuDH derived from Pseudomonas aeruginosa PAO1, which catalyzes the reversible deamination of branched chain amino acids (i.e., leucine, valine and isoleucine),

the branched chain amino acid aminotransferase ilvE from E. coli Nissle, which catalyzes the reversible deamination of branched chain amino acids (i.e., leucine, valine and isoleucine),

the ketoacid decarboxylase kivD derived from Lactococcus lactis strain IFPL730, which catalyzes the decarboxylation of branched chain amino acids, and/or

the alcohol dehydrogenase adh2 derived from Saccharomyces cerevisiae, which catalyzes the conversion of branched chain amino acid-derived aldehydes to their respective alcohols.

Specifically, the following constructs were generated: Ptet-kivD (SYN479), ptet-kivD-leuDH (SYN467), Ptet-kivD-adh2 (SYN949), ptet-leuDH-kivD-adh2 (SYN954), and Ptet-ilvE-kivD-adh2 (SYN950).

SYN467, SYN469, SYN479, SYN949, SYN950 and SYN954 were grown overnight at 37° C. and 250 rpm in 4 mL of LB supplemented with 100 μg/mL kanamycin for SYN467, SYN479, SYN949, SYN950 and SYN954. Cells were diluted 100 fold in 4 mL LB (with 100 μg/mL kanamycin for SYN467, SYN479, SYN949, SYN950 and SYN954) and grown for 2 h at 37° C. and 250 rpm. Cells were split in two 2 mL culture tubes, and one 2 mL culture tube was induced with 100 ng/mL anhydrotetracycline (ATC) to activate the Ptet promoter. After 1 h induction, the two 2 mL culture tubes were split in four 1 mL microcentrifuge tubes. The cells were spun down at maximum speed for 30 seconds in a microcentrifuge. The supernatant was removed and the pellet re-suspended in 1 mL M9 medium 0.5% glucose. The cells were spun down again at maximum speed for 30 seconds and resuspended in 1 mL M9 medium 0.5% glucose supplemented with 2 mM leucine or 2 mM KIC. Serial dilutions of the different cell suspensions were plated to determine the initial number of CFUs. The cells were transferred to a culture tube and incubated at 37° C. and 250 rpm for 3 h. 150 μL of cells were collected at 0 h, 1 h, 2 h and 3 h after addition of leucine or KIC for quantification by LC-MS/MS. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2 in positive mode

KIC: 129.1/129.1 in negative mode

The rate of degradation (in μmol/10⁹ CFUs/hr) was calculated for leucine and KIC.

FIG. 24 and FIG. 25 demonstrate that the different recombinant bacteria are able to debrade both leucine and KIC. The best performing strain was SYN950, with a 0.8 and 2.2 μmol/10⁹ CFUs/hr degradation rate for leucine and KIC, respectively.

The following table summarizes other experimental data generated in the course of evaluating leucine-degrading circuits:

TABLE 14

| Feature | Insights Gained |
|---|---|
| Branched chain aa recycling | Intrinsic production of valine by |
| (E. coli can synthesize | engineered strain does not interfere with |
| and excrete valine) | leucine degradation |
| Gene expression level | High copy expression of kivD enhances |
| | degradation rates → seeking switches with |
| | stronger activation levels |
| Co-factor requirement | Adding exogenous thiamine does not |
| | increase activity → endogenous pools |
| | sufficient |
| Environmental and assay pH | pH optimum = 6.5 → reaction should |
| | work well under GI and physiological |
| | conditions |
| Carbon source utilization and | Glucose drives optimal reaction rates → |
| byproducts | no evidence for inhibition by glycolysis |
| | (e.g., acid) byproducts |

Additional measures that may be taken to improve branched chain amino acid degradation rate include:

| Potential limitation | Test |
|---|---|
| BCAA uptake by | Test additional BCAA transporters |
| cell is rate- | Establish genetic selections for transporter |
| limiting | mutants with increased activity |
| BCAA-derived | Increase ADH2 expression/activity to convert the |
| aldehydes | aldehydes into their respective alcohol |
| inhibit KivD | |
| Slow conversion | Express leuDH on a separate transcript from kivD |
| of BCAAs into | Increase transcription rates for leuDH and kivD |
| their ketoacids | Overexpress the endogenous ilvE (BCAT) |
| | Identify KivD or LeuDH variants with increased |
| | enzymatic activity |
| Slow folding or | Increase cellular osmolytes concentration |
| misfolding | (NaCl + betaine) |
| of BCDH or | Lower induction temperature |
| KivD | Induce the expression of endogenous chaperones |
| | (heat-shock, benzyl alcohol) |
| | Express chaperones (dnaK-dnaJ-grpE, groES- |
| | groEL) |

### Example 8. Construction of Plasmids Encoding Branched Chain Amino Acid Catabolism Enzymes, Including a BCAA Deaminating Enzyme, an Alpha-Keto-Acid Decarboxylase, an Alcohol Dehydrogenase or an Aldehyde Dehydrogenase

The genes encoding the leucine dehydrogenases LeuDH_{Pa}. (SEQ ID NO: 19) from Pseudomonas aeruginosa, the leucine dehydrogenase LeuDH_{Bc} (SEQ ID NO: 58) from Bacillus cereus, the L-amino acid deaminase LAAD_{PV} (SEQ ID NO: 56) from Proteus vulgaris, the alcohol dehydrogenase Adh2 (SEQ ID NO: 38) from S. cerevisae, the alcohol dehydrogenase YqhD (SEQ ID NO: 60) from E. coli Nissle and the aldehyde dehydrogenase PadA (SEQ ID NO: 62) from E. coli K12 were incorporate into the pTet-kivD (SEQ ID NO: 2) plasmid described herein by Gibson assembly to generate the following constructs: pTet-kivD-leuDH_{Pa}, pTet-kivD-adh2, pTet-LeuDH_{Pa}-kivD-adh2, pTet-LeuDH_{Bc}-kivD-adh2, pTet-LeuDH_{Pa}-kivD-yqhD, pTet-LeuDH_{Bc}-kivD-yqhD, pTet-LeuDH_{Pa}-kivD-padA, pTet-LeuDH_{Bc}-kivD-padA, pTet-Laad_{Pv}-kivD-adh2, pTet-Laad_{Pv}-kivD-yqhD, pTet-Laad_{Pv}-kivD-padA. Those constructs were transformed into the following E. coli Nissle strains described herein: ΔleuE, ΔleuE lacZ:tet-livKHMGF and ΔleuE ΔilvC lacZ:tet-livKHMGF

### Example 9. Improved Degradation of Leucine in Recombinant Bacteria Expressing Branched Chain Amino Acid Catabolism Enzyme by Expressing an Importer of Branched Chain Amino Acid

In these studies, all incubations were performed at 37° C. Cultures of E. coli Nissle ΔleuE lacZ:Tet-livKHMGF and Nissle ΔleuE lacZ:Tet-livKHMGF, pTet-kivD were grown overnight LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD (SEQ ID NO: 2) and LivKHFMG (SEQ ID NO: 10), and bacteria were grown for another 3 hours. Bacteria were then pelleted, washed, and resuspended in minimal media to an OD₆₀₀ of 1 and supplemented with 0.5% glucose and 2 mM leucine. Aliquots were removed at 0 h and 4 h for leucine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

Isoleucine: 132.1/86.2

Valine: 118.1/72

The rate of leucine degradation was calculated based on the number of CFUs (colony forming units) determined at T0 by plating serial dilution on LB plates.

As shown in FIGS. 42A and 42B, leucine is consumed without the presence of ATC, due to normal bacterial growth during the assay. In the presence of ATC, degradation is further improved by the expression of livKHMGF and kivD.

### Example 10. Degradation of all Three Branched Chain Amino Acids by Recombinant Bacteria Expressing Branched Chain Amino Acid Catabolism Enzyme and Improved Degradation of Leucine by Expressing a Leucine Dehydrogenase

In these studies, all incubations were performed at 37° C. Cultures of E. coli Nissle ΔleuE lacZ:Tet-livKHMGF with the pTet-kivD or pTet-kivD-leuDH_{Pa} plasmid, were grown overnight in LB, LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD (SEQ NO:2), LeuDH_{Pa}. (SEQ ID NO: 20) and LivKHMGF, and bacteria were grown for another 3 hours. Bacteria were then pelleted, washed, and resuspended in minimal media to OD₆₀₀ of 1, and supplemented with 0.5% glucose and the three branched chain amino acids (leucine, isoleucine and valine, 1 mM each). Aliquots were removed at 0 h, 3 h, 19 h for leucine, isoleucine and valine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

Isoleucine: 132.1/86.2

Valine: 118.1/72

The rate of leucine degradation was calculated based on the number of CFUs (colony forming units) determined at T0 by plating serial dilution on LB plates.
As shown in FIGS. 43A, 43B and 43C, leucine, isoleucine and valine were all degraded by the expression of kivD and kivD-leuDH_{Pa} in E. coli Nissle. The efficiency of leucine degradation was improved 25% by expressing the leucine dehydrogenase leuDH_{Pa} (FIG. 43D).

### Example 11. Enhanced Degradation of Leucine by Recombinant Bacteria Expressing an L-Amino Acid Deaminase

In these studies, all incubations were performed at 37° C. Cultures of E. coli Nissle ΔleuE ΔilvC lacZ:Tet-livKHMGF (SYN469) with the pTet-ilvE-kivD-adh2, pTet-LeuDH_{Pa}-kivD-adh2 or pTet-Laad_{Pv}-kivD-leuDH_{Pa} plasmid, were grown overnight in LB for SYN469 and 50 ug/mL Kanamycin for strains containing a plasmid and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD (SEQ ID NO: 2), LeuDH_{Pa} (SEQ ID NO: 20), IlvE (SEQ ID NO: 22), LAAD_{PV} (SEQ ID NO: 24) and LivKHFMG, and bacteria were grown for another 3 hours. Bacteria were then pelleted, washed, and resuspended in minimal media to OD₆₀₀ of 1, and supplemented with 0.5% glucose and 2 mM. Aliquots were removed at 0 h and 3 h for leucine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

The rate of leucine degradation was calculated based on the number of CFUs (colony forming units) determined at T0 by plating serial dilution on LB plates.

FIG. 47B depicts the leucine degradation pathway used in the strains tested. As shown in FIG. 47A, leucine degradation is greatly enhanced by the expression of LAAD_{PV} (15-fold). This efficiency of leucine degradation far exceed the upper target degradation rate for efficient treatment of MSUD described herein and marked by a dotted line in FIG. 47A.

### Example 12. Degradation of Leucine by Recombinant Bacteria Expressing L-Amino Acid Deaminases from Proteus vulgaris and Proteus mirabilis

The gene encoding the L-amino acid deaminase Pma from Proteus mirabilis LAAD_{Pm} (SEQ ID NO: 26) was cloned under the control of the tet promoter in the high copy plasmid pUC57-Kan to generate the pTet-Laad_{Pm} plasmid. The pTet-Laad_{Pm} plasmid was transformed in the ΔleuE ΔilvC lacZ:Tet-livKHMGF (SYN469). In these studies, all incubations were performed at 37° C. Cultures of E. coli Nissle ΔleuE ΔilvC lacZ:Tet-livKHMGF (SYN469) with the pTet-Laad_{Pv}-kivD-adh2, pTet-Laad_{Pv}-kivD-yqhD, pTet-Laad_{Pv}-kivD-padA or pTet-Laad_{Pm} plasmid, were grown overnight in LB for SYN469 and 50 ug/mL Kanamycin for strains containing a plasmid and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of the constructs, and bacteria were grown for another 3 hours. Bacteria were then pelleted, washed, and resuspended in minimal media to OD₆₀₀ of 1, and supplemented with 0.5% glucose and 2 mM leucine. Aliquots were removed at 0 h and 3 h for leucine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

The rate of leucine degradation was calculated based on the number of CFUs (colony forming units) determined at T0 by plating serial dilution on LB plates.

FIG. 48B depicts the leucine degradation pathway used in the strains tested. As shown in FIG. 48A, leucine degradation occurs at very efficient rates in strains expressing either LAAD_{PV} or LAAD_{Pm}. The efficiency of leucine degradation far exceeds the upper target degradation rate for efficient treatment of MSUD described herein and marked by a dotted line in FIG. 48A.

### Example 13. Improvement of Leucine Degradation by Recombinant Bacteria Expressing BCAA Catabolism Enzymes and a Leucine Importer

In these studies, all incubations were performed at 37° C. Cultures of E. coli Nissle ΔleuE lacZ:Tet-livKHMGF (SYN452) and ΔleuE (SYN458) with or without the pTet-LeuDH_{Pa}-kivD-padA plasmid, were grown overnight in LB for SYN452 and SYN458 or LB with 50 ug/mL Kanamycin for strains containing a plasmid and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of the constructs, and bacteria were grown for another 3 hours. Bacteria were then pelleted, washed, and resuspended in minimal media, and supplemented with 0.5% glucose and 4 mM leucine. Aliquots were removed at T0, 40 min, 90 min and 150 min for leucine, KIC and isovaleric acid (IVA) quantification by liquid chromatography-mass spectrometry (LCMS) using a Thermo TSQ Quantum Max triple quadrupole instrument. The rate of leucine degradation, KIC and IVA production was calculated based on the number of CFUs (colony forming units) determined at T0 by plating serial dilution on LB plates. FIG. 49B depicts the leucine degradation pathway used in the strains tested. As shown in FIG. 49A, the expression of livKHMGF in SYN452 moderately improves the rate of leucine degradation in comparison with SYN458. This correlates with a mild increase in the production of isovalerate.

### Example 14. Improvement of Leucine Degradation by Recombinant Bacteria Expressing the Leucine Dehydrogenase from Bacillus cereus and the Low Affinity BCAA Transporter BrnQ

The gene encoding the leucine dehydrogenase from Bacillus cereus (LeuDHBc) (SEQ ID NO: 58) was cloned in place of the leucine dehydrogenase from Pseudomonas aeruginosa leuDHPa (SEQ ID NO: 20) in the pTet-leuDHPa-kivD-padA constructs by Gibson assembly to generate the pTet-leuDHBc-kivD-padA plasmid. This plasmid was transformed into the E. coli Nissle ΔleuE ΔilvC lacZ:Tet-livKHMGF (SYN469) strain. The gene encoding E. coli Nissle low-affinity transporter BrnQ (SEQ ID NO: 64) was cloned under the control of the tet promoter in the low-copy plasmid pSC101 by Gibson assembly. The generated pTet-brnQ plasmid was transformed into the newly generated E. coli Nissle ΔleuEAilvC, lacZ:Tet-livKHMGF, pTet-leuDHBc-kivD-padA strain to generated the ΔleuEAilvC, lacZ:Tet-livKHMGF, pTet-leuDHBc-kivD-padA, pTet-brnQ strain. In these studies, all incubations were performed at 37° C. Cultures of E. coli Nissle ΔleuEAilvC,lacZ:Tet-livKHMGF, pTet-leuDHPa-kivD-padA, E. coli Nissle ΔleuEAilvC,lacZ:Tet-livKHMGF, pTet-leuDHBc-kivD-padA and E. coli Nissle ΔleuEAilvC,lacZ:Tet-livKHMGF, pTet-leuDHBc-kivD-padA, pTet-brnQ strains were grown overnight in LB with 50 ug/mL Kanamycin and 100 ug/mL carbenicillin for the for strain containing pTet-brnQ. The cells were grown with shaking (250 rpm) to early log phase with the appropriate antibiotics. Anhydrous tetracycline (ATC) was added to cultures at a concentration of 100 ng/mL to induce expression of the constructs, and bacteria were grown for another 3 hours. Bacteria were then pelleted, washed, and resuspended in minimal media, and supplemented with 0.5% glucose and 4 mM leucine. Aliquots were removed at T0, 1 h, 2 h and 3 h for leucine, KIC and isovaleric acid (IVA) quantification by liquid chromatography-mass spectrometry (LCMS) using a Thermo TSQ Quantum Max triple quadrupole instrument. The rate of leucine degradation, KIC and IVA production was calculated based on the number of CFUs (colony forming units) determined at T0 by plating serial dilution on LB plates. FIG. 50C depicts the leucine degradation pathway used in the strains tested. As shown in FIG. 50A, the expression of leuDHBc doubles the rate of leucine degradation compare to leuDHPa. The expression of the low-affinity BCAA transporter BrnQ dramatically improves the rate of leucine degradation, by 4 to 5 fold. In both cases, the increased level of leucine degradation correlates with an increased level of isovalerate production as shown in FIG. 50A. The expression of BrnQ also leads to the accumulation of KIC, suggesting that the decarboxylation of KIC by kivD becomes the limiting step in the pathway. The efficiency of leucine degradation obtained by expressing BrnQ exceeds the upper target degradation rate for efficient treatment of MSUD described herein and marked by a dotted line in FIG. 50B.

### Example 15. Recirculation of Isotopic Leucine into the Mouse Intestine after Subcutaneous Injection

To understand the kinetic relationship between intestinal and systemic levels of exogenously administered leucine, and determine if subcutaneous injection of leucine can be used as an acute model of MSUD to assess the activity of leucine-degrading strains, heavy isotope-labeled leucine (¹³C₆) was injected subcutaneously at 0.1 mg/g in BL6 mice and quantified in plasma, small intestine effluent, cecum and large intestine effluent at different times after injection (before injection (T0), 30 min, 1 h and 2 h after injection). For each time point, 3 mice were bled and dissected to collect their small intestine, cecum and large intestine content. ¹³C₆-Leu was quantified by LC-MS/MS by liquid chromatography-mass spectrometry (LCMS) using a Thermo TSQ Quantum Max triple quadrupole instrument. Briefly, 10 uL of samples were resuspended in 90 uL of derivatization mix (50 mM 2-Hydrazinoquinoline, 50 mM triphenylphosphine, 50 mM, 2,2′-dipyridyl disulfide in acetonitrile) with 1 ug/mL L-leucine-5,5,5-d₃ (isotope used as internal standard). The samples were then incubated at 60° C. for 1 h, centrifuged at 4,500 rpm at 4° C. for 5 min 20 uL was then transferred to 180 uL of water, 0.1% formic acid and placed in the LCMS autosampler. A C18 column 50×2 mm, Sum particles was used (Luna, Phenomenex). The mobile phases used were water 0.1% formic acid (solvent A) and acetonitrile 0.1% (solvent B). The mass spectrometer was run in positive mode and the Q1/Q3 transitions used for ¹³C₆-Leu quantification were 279.1/144.2 and 279.1/160.2. FIG. 53 shows that ¹³C₆-Leu is present in the plasma and the small intestine as early as 30 min after injection, demonstrating that leucine is able to recirculate from the periphery into the small intestine. After 30 min, the level gradually decreases. ¹³C₆-Leu remains undetectable in the cecum and the large intestine, suggesting that leucine is not able to recirculate to those parts of the gastrointestinal tract. Those results demonstrate that an increase in plasma leucine level, mimicking a transient acute MSUD state, can be obtained by subcutaneous injection of leucine and that part of this leucine can become available for an engineered BCAA-degrading bacteria residing in the gastrointestinal tract.

### Example 16. Testing the Efficacy of Engineered BCAA-Degrading Bacteria in an Acute Model of MSUD

Intermediate MSUD mice are kept on a 50/50 BCAA-free diet (Dyets)/18% protein chow (Teklad), in order to maintain a normal level of BCAA in those animals and prevent mortality. All animals are bled before being injected with a mix of three BCAA amino acids subcutaneously in order to mimic an acute episode of high BCAA in those animals. After injection, animals are gavaged with a control bacterial strain, which is unable to degrade BCAA, or an BCAA-degrading strain, or a mock control made of the formulation buffer used to prepare bacterial inocula. At different time after gavaging, plasma is collected, and the level of each BCAA is determined to measure the efficacy of the treatment in reducing the systemic level of BCAAs. In one embodiment, the brain of the animals are collected to measure BCAAs. In another embodiment, the urine of the animals is collected to measure BCAAs.

In a second instance, intermediate MSUD mice are kept on a 50/50 BCAA-free diet (Dyets)/18% protein chow (Teklad), in order to maintain a normal level of BCAA in those animals and prevent mortality. All animals are bled before changing their diet to a chow with 10%, 15%, 18%, 20%, 30%, 40%, 50%, 60% or 70% proteins. After changing their diet, animals are gavaged with a control bacterial strain, unable to degrade BCAA, or an BCAA-degrading strain, or a mock control made of the formulation buffer used to prepare bacterial inocula. At different time after gavaging, plasma is collected, and the level of each BCAA is determined to measure the efficacy of the treatment in reducing the systemic level of BCAAs. In one embodiment, the brain of the animals are collected to measure BCAAs. In another embodiment, the urine of the animals is collected to measure BCAAs.

### Example 17. Increase of BCAA Import by Overexpressing the High Affinity BCAA Transporters livKHMGF and livJHMGF In Vitro

In these studies, all the strains are derived from the human probiotic strain E. coli Nissle ΔleuE. In the ΔleuE, lacZ:Ptet-livKHMGF strain, the endogenous promoter of IivJ was swapped with the constitutive promoter Ptac by lambda-red recombination using the Ptac-livJ construct (SEQ ID NO: 11) to generate the ΔleuE, lacZ:Ptet-livKHMGF, Ptac-livJ strain. In this strain, livJ is constitutively induced. In the presence of ATC, both BCAA transporters livKHMGF and livJHMGF are expressed. ΔleuE; ΔleuE, lacZ:Ptet-livKHMGF; ΔleuE, lacZ:Ptet-livKHMGF, Ptac-livJ strains were grown overnight at 37° C. and 250 rpm in 4 mL of LB. Bacterial Cells were then diluted 100 fold in 4 mL LB and grown for 2 h at 37° C. and 250 rpm. Cells were then split in two 2 mL culture tubes. One 2 mL culture tube was induced with 100 ng/mL anhydrotetracycline (ATC) to activate the Ptet promoter. After 1 h induction, 1 mL of cells was spun down at maximum speed for 30 seconds in a microcentrifuge. The supernatant was then removed and the pellet re-suspended in 1 mL M9 medium 0.5% glucose. The cells were spun down again at maximum speed for 30 seconds and resuspended in 1 mL M9 medium 0.5% glucose. The cells were then transferred to a culture tube and incubated at 37° C. and 250 rpm for 5.5 h. 150 μL of cells were collected at 0 h, 2 h and 5.5 h and the concentration of valine in the cell supernatant at the different time points was determined by LC-MS/MS using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used is:

Valine: 118.1/72

As FIG. 53 shows, the natural secretion of valine by E. coli Nissle is observed for the ΔleuE strain. The secretion of valine is strongly reduced for ΔleuE, lacZ:Ptet-livKHMGF in the presence of ATC. This strongly suggests that the secreted valine is efficiently imported back into the cell by livKHMGF. The secretion of valine is abolished in the ΔleuE, lacZ:Ptet-livKHMGF, Ptac-livJ strain, with or without ATC. This strongly suggests that the constitutive expression of livJ is sufficient to import back the entire amount of valine secreted by the cell via the livJHMGF transporter. In conclusion, we successfully engineered E. coli Nissle to efficiently import BCAA, in this case valine, using both an inducible promoter (Ptet), and a constitutive promoter (Ptac), controlling the expression of livKHMGF and livJ respectively.

### Example 18. Improved Transport of Leucine in Recombinant Bacteria Expressing a Leucine Importer

In order to test if expressing the high-affinity leucine transporter livKHMGF increases the transport of leucine into the bacterial cell, the minimum inhibitory concentration (MIC) of the toxic analog 3-fluoroleucine was determined for the following E. coli Nissle strains: E. coli Nissle, ΔleuE and ΔleuE, lacZ:Tet-livKHMGF. Those strains were grown overnight in LB and diluted 2,000 fold in M9 minimum media supplemented with 0.5% glucose, in the presence of 250, 125, 62.5, 31.2, 15.6, 7.8, 3.9, 2, 1 or 0 ug/mL 3-fluoroleucine in the presence or absence of 100 ng/mL ATC for ΔleuE, lacZ:Tet-livKHMGF. Cells were grown at 37° C. for 20 h. The MIC for each strain, with our without ATC, was determined by looking at the presence or absence of bacterial growth for each treatment and was defined as the minimum concentration blocking bacterial growth. The following Table 15 describes the results:

TABLE 15

MIC (ug/mL)

| Strain | −ATC | +ATC |
|---|---|---|
| Nissle | 31.25 | ND |
| ΔleuE | 62.5 | ND |
| ΔleuE, lacZ:Tet-livKHMGF | 31.25 | 2 |

The induction of the leucine importer livKHMGF by ATC in the ΔleuE, lacZ:Tet-livKHMGF strain led to a 16-fold reduction in the MIC to 3-fluoroleucine, going from 31.25 to 2 ug/mL. This dramatic increase in sensitivity to the leucine toxic analog demonstrates that the expression of livKHMGF leads to a substantial increase in leucine transport into the cell.

### Example 19. In Vitro Activity of Leucine Consuming Strains (with or without a Low-Copy ATC-Inducible brnQ Construct)

To test the low-copy ATC-inducible constructs and confirm the effect of brnQ on leucine degradation, strains were generated (according to methods described in Example 1 and others) as follows and tested for in vitro leucine degradation activity. SYN1992 comprises ΔleuE, ΔilvC, a tet inducible livKHMGF construct integrated into the bacterial chromosome at the LacZ locus, and a tet inducible leuDH(Bc)-kivD-adh2-rrnB ter construct on a low copy plasmid (ΔleuE, ΔilvC, lacZ:tetR-Ptet-livKHMGF, tetR-Ptet-leuDH(Bc)-kivD-adh2-rrnB ter (pSC101)). SYN1980 comprises ΔleuE, ΔilvC, a tet-inducible livKHMGF construct integrated at the lacZ locus in the bacterial chromosome, and a tet-inducible leuDH(Bc)-kivD-adh2-brnQ-rrnB ter construct on a low copy plasmid (ΔleuE, ΔilvC, lacZ:tetR-Ptet-livKHMGF, tetR-Ptet-leuDH(Bc)-kivD-adh2-brnQ-rrnB ter (pSC101)). The organization of the construct is depicted in FIG. 54C and comprises SEQ ID NO: 121 (LeuDH-kivD-adh2-brnQ). SYN469, comprising ΔleuE, ΔilvC, and integrated lacZ:tetR-Ptet-livKHMGF, was used as a control.

Overnight cultures were subcultured 1/100 in 5 mL LB plus carbenicillin (except for SYN469) and grown for 3 h at 37 C, 250 rpm. Cultures were either left uninduced or induced for 2 hours with ATC 100 ng/mL Bacteria (1 ml) were spun down, washed with 1 mL of M9 plus 0.5% glucose, and resuspended 1 mL of M9 medium with 0.5% glucose and 4 mM leucine. Bacteria concentration was determined using a cellometer. Bacteria were transferred to culture tubes (at 37 C, 250 rpm) and samples were taken at 1.5 and 3 h, leucine concentrations measured and degradation rates calculated. Results are shown in FIG. 54A and FIG. 54B. Leucine degradation was increased in both SYN1992 and SYN1980 upon addition of tetracycline, with SYN1980 (comprising tet-inducible BrnqQ) having a greater degradation rate.

### Example 20. In Vitro Activity of Leucine Consuming Strains (with or without a Low-Copy FNR-Inducible brnQ Construct)

To test low copy no/low oxygen inducible FNR driven constructs and confirm the effect of brnQ on leucine degradation, strains were generated (according to methods described in Example 1 and others) as follows and tested for in vitro Leucine degradation activity.

SYN1993 comprises ΔleuE, ΔilvC, a tetracycline inducible livKHMGF construct integrated into the LacZ locus of the bacterial chromosome, and a low/no oxygen inducible, FNR driven leuDH(Bc)-kivD-adh2-rrnB ter construct on a low copy plasmid (SYN1993: ΔleuE, ΔilvC, lacZ:tetR-Ptet-livKHMGF, PfnrS-leuDH(Bc)-kivD-adh2-rmB ter (pSC101)). SYN1981 comprises ΔleuE, ΔilvC, a tetracycline inducible livKHMGF construct integrated into the LacZ locus of the bacterial chromosome, and a low/no oxygen inducible, FNR driven leuDH(Bc)-kivD-adh2-brnQ-rrnB ter construct on a low copy plasmid (SYN1981: ΔleuE, ΔilvC, lacZ:tetR-Ptet-livKHMGF, PfnrS-leuDH(Bc)-kivD-adh2-brnQ-rrnB ter (pSC101)). The organization of the construct is depicted in FIG. 55C and comprises SEQ ID NO: 121 (LeuDH-kivD-adh2-brnQ). SYN469, comprising ΔleuE, ΔilvC, and integrated tetR-Ptet-livKHMGF at the LacZ locus, was used as a control.

Overnight cultures were subcultured 1/100 in 5 mL LB plus carbenicillin (except for SYN469) and grown for 3 h at 37 C, 250 rpm. Cultures were either left uninduced or transferred to an Coy anaerobic chamber supplying 90% N₂, 5% CO₂, and 5% H₂. Bacteria (1 ml) were spun down, washed with 1 mL of M9 plus 0.5% glucose, and resuspended 1 mL of M9 medium with 0.5% glucose and 4 mM leucine. Bacterial concentration was determined using a cellometer. Bacteria were transferred to culture tubes (at 37 C, 250 rpm), samples were taken at 1.5 and 3 h and leucine concentrations measured and degradation rates calculated. Results are shown in FIG. 55A and FIG. 55B. Leucine degradation was increased in both SYN1993 and SYN1981 upon anaerobic induction, with SYN1981 (comprising FNR inducible BrnqQ) having a greater degradation rate.

### Example 21. In Vivo Efficacy Study for BCAA Consuming Strain SYN1980

The ability of engineered strain SYN1980 (comprising ΔleuE, ΔilvC, lacZ:tetR-Ptet-livKHMGF, tetR-Ptet-leuDH(Bc)-kivD-adh2-brnQ-rrnB ter (in low-copy pSC101 plasmid) to decrease plasma BCAA levels was tested in vivo in the intermediate MSUD (iMSUD) animal model (as described in Zinnanti et al., Dual mechanism of brain injury and novel treatment strategy in maple syrup urine disease; Brain 2009: 132; 903-918). SYN1980 was compared to wild type Nissle with a streptomycin resistance in this study.

To prepare the cells for this study, bacterial growth and induction conditions were as follows. Overnight cultures (5 mL) with Strep (control Nissle) or Carbenicillin (SYN1980). 500 mL LB flasks were inoculated with the overnight cultures, and grown for 2 h at 37 C with 250 rpm. Next anhydrotetracycline (ATC 100 ng/mL) was added for 2 hours. Cultures were spun down at 4 C for 30 min, at 4,000 rpm and the pellets were resuspended in 10 mL formulation buffer (PBS, 15% glycerol, 2 g/L glucose, 3 mM thymidine), aliquoted in 2 ml cryovials and kept at −80 C.

iMSUD mice (6-10 weeks of age) were kept on BCAA free chow (Dyets 510081, Bethlehem, Pa., USA) mixed 1:1 with 18% protein chow (2018 Teklad global 18% protein diets) and water. On day 1, animals were randomized into treatment groups. Mice were bled and (T=0) to obtain baseline plasma BCAA levels. Mice were grouped as follows: Group 1: vehicle control (formulation buffer) (n=10); Group 2: wild type Nissle with streptomycin resistance (n=10); Group 3: SYN1980 strain (n=10); For Groups 2 and 3, mice were gavaged with ˜2e9 CFUs/dose in 200 μl/ose in the am and pm (2 doses per day). For group 3, ATC (20 ng/mL) and 5% sucrose was added to the drinking water. Group 1 was dosed with 200 ul formulation buffer. At the end of the day, mice were placed on high protein chow (70% protein, 5% carbohydrate, and 8% fat, TD150582, Harlan Laboratories) plus 5% sucrose. Mice were continued on high protein chow throughout the study.

On day 2, mice were dosed twice daily with 200 ul bacteria (˜2e9 CFU/dose) or formulation buffer (Group 1). On day 3, the mice were placed on BCAA-free chow in the morning and dosed three times with 200 ul bacteria (˜2e9 CFU/dose) or formulation buffer with one hour intervals. Animals were weighed and blood was collected at 1 hour post last dose and stored on ice for LC/MS analysis. Animals were sacrificed and brains were extracted, ground in 1 mL 10% ACN to test the BCAA levels and stored on ice for LC/MS analysis.

Results are shown in FIG. 56. Levels of Leu and Val remained lower in the plasma of SYN1980-treated animals, resulting in a lower ΔLeu and ΔVal (FIG. 56A, FIG. 56B, FIG. 56D, FIG. 56E), as compared to animals treated with streptomycin resistant Nissle or vehicle control, where the switch to high protein diet lead to increased levels Leu and Val. A similar trend of lower Leu and Val and reduced ΔLeu and ΔVal was found in the brain (FIG. 56G, FIG. 56H). No significant changes in Ile concentrations in plasma or brain were observed; the switch to high protein chow did not seem to increase Ile levels in the iMSUD mice (FIG. 56C, FIG. 56F, and FIG. 56I), consistent with the observations described in Zinnanti et al for the iMSUD model.

### Example 22. In vivo Efficacy Study for BCAA Consuming Strain SYN1980

Next, the ability of the BCAA consuming strain SYN1980 to reduce and/or prevent the neurological phenotype seen in iMSUD mice was tested. The study was repeated essentially as described in Example 21 with 2 mice per group, except that on day 3, mice were dosed twice daily with 200 ul bacteria (˜2e9 CFU/dose) or formulation buffer (Group 1), and animal movement was recorded for 5 minutes. One mouse gavaged with SYN1780 died during this study, due to unrelated causes during the study procedure. Videos were scored for number of amulations, and results are shown in FIG. 57. The surviving mouse gavaged with SYN1980 showed reduced activity on day 3 as compared to day 1, but significantly greater activity than mice gavaged with streptomycin resistant E. coli

### Example 23. In Vivo Efficacy Study for BCAA Consuming Strain SYN1980

Next, the ability of the BCAA consuming strain SYN1980 to reduce and/or prevent the neurological phenotype seen in iMSUD mice the experiment is further studied. The study is repeated essentially as in Example 21, except that on day 3, mice are dosed twice daily with 200 ul bacteria (˜1e9 CFU/dose) or formylation buffer (Group 1). On day 4, mice are dosed three times with 200 ul bacteria (˜1e9 CFU/dose) or water with one hour intervals. Animals are weighed and blood is collected at 1 hour post last dose and stored on ice for LC/MS analysis. Animals are sacrificed and brains are extracted, ground in 1 mL 10% ACN to test the BCAA levels are stored on ice for LC/MS analysis. Additionally, Group 4, which is provided with high protein diet with 5% norleucine throughout the study, is added as another control. The addition of norleucine is expected to reduce the neurological phenotype (Zinnanti et al.).

On days 2, 3, and 4, animals and controls are assessed for motor deficits using a quantitative neurological scale (as described in Zinnanti et al., Dual mechanism of brain injury and novel treatment strategy in maple syrup urine disease; Brain 2009: 132; 903-918, and references therein). To assess improvements in motor abnormalities in the mice administered the genetically engineered bacteria, motor abnormalities are scored on the presence and severity of motor symptoms consisting of intermittent dystonia of one hindlimb, intermittent dystonia of two hindlimbs, permanent dystonia of hindlimbs, gait abnormalities i.e., wobbling gait, frequent falls or rolls, recumbency (i.e., paralysis with rapid breathing). Additionally, improvements in the grasp reflex of forepaws and hindpaws is assessed and included in the score. Cage hang tests are performed by placing mice on a wire mesh cage lid and inverting the lid. The time that the mouse could hang upside down without falling is recorded over three trials (as described in Zinnanti et al.).

### Example 24. In Vivo Efficacy Studies for BCAA Consuming Strains

Efficacy of various BCAA consuming strains are assessed in the iMSUD mouse model described in the previous examples.

Integrated Strains

Next, the ability of an BCAA consuming engineered strain, in which the BCAA catabolism enzymes and the BCAA transporter BRNQ are integrated into the bacterial chromosome and are under the control of the no/low oxygen inducible FNR promoter, to decrease plasma BCAA levels is tested in vivo in the intermediate MSUD (iMSUD) animal model described in the previous examples. The strain comprises the following cassettes, each integrated into the bacterial chromosome, e.g., at one or more sites shown in FIG. 68B: ΔleuE, ΔilvC, PFNR-livKHMGF, FNRleuDH(Bc)-kivD-adh2-brnQ-rrnB. SYN-2016 is compared to wild type Nissle with a streptomycin resistance in this study.

The study is carried out essentially as described in Examples 21 and Example 22 and leucine, valine and isoleucine levels are measured in plasma and the brain. Motor deficits are quantified using the neurological scale as described above.

Strains with Plasmid Based Safety Switch

Next the following strains are generated and tested in the iMSUD model. Strains are generated as described herein and using methods known in the art, using the plasmid based safety switch system as described herein. A first genetically engineered strain comprises ΔleuE, ΔilvC, and an tet-inducible livKHMGF construct, integrated into the bacterial chromosome, e.g., at the LacZ locus, and a construct shown in FIG. 67C. In a second strain, the construct shown in FIG. 67D is used in lieu of the construct shown in FIG. 67C. The first and second strains further comprise a plasmid shown in FIG. 67A, except that the bla gene is replaced with a construct comprising codon optimized LeuDH-kivD-adh2-brnQ driven by an FNRS promoter (see, e.g., FIG. 55C).

A third genetically engineered strain comprises ΔleuE, ΔilvC, and a tet inducible livKHMGF construct, integrated into the bacterial chromosome, e.g., at the LacZ locus, and a construct shown in FIG. 67C knocked into the dapA locus on the bacterial chromosome. In a fourth strain, the construct shown in FIG. 67D is used in lieu of the construct shown in FIG. 67C. The third and fourth strains further comprise a plasmid shown in FIG. 67A, except that the bla gene is replaced with a construct comprising codon optimized LeuDH-kivD-adh2-brnQ driven by a tet promoter (see, e.g., FIG. 54C).

In alternate embodiments, a plasmid based safety switch system for ThyA auxotrophy is used in lieu of the system for dapA auxotrophy.

The study is carried out essentially as described in Examples 21 and Example 22 and leucine, valine and isoleucine levels are measured in plasma and the brain. Motor deficits are quantified using the neurological scale as described above.

### Example 25. Leucine, Isoleucine, and Valine Quantification in Plasma and Brain Tissue by LC-MS/MS

### Sample Preparation

Leucine, Isoleucine, and Valine stock (10 mg/mL) was prepared in water and aliquoted into 1.5 mL microcentrifuge tubes (100 μL). Standards (500, 250, 100, 20, 4, 0.8, 0.16, 0.032 μg/mL) of each was prepared in water. Whole brain tissues were homogenized with 1 mL of 10% ACN/0.1% formic acid in water in BeadBug prefilled tubes using a FastPrep homogenizer. The brain homogenate samples were transferred into a V-bottom 96-well plate and centrifuged at 4000 rpm for 10 min Plasma samples were centrifuged at 4000 rpm for 5 min Samples and standards (10 μL) were mixed with 90 μL of 60:30 (ACN/water) containing 1 μg/mL of Leu-d3 (used as internal standard) in the final solution in a V-bottom 96-well plate. The plate was heatsealed with a AlumASeal foil, mixed well, and centrifuged at 4000 rpm for 5 min. The solution (20 μL) was transferred into a round-bottom 96-well plate and 180 uL 0.1% formic acid in water was added to the sample. The plate was heatsealed with a ClearASeal sheet and mixed well.

### LC-MS/MS Method

Leucine, Isoleucine, and Valine were measured by liquid chromatography coupled to tandem mass spectrometry (LC-MS/MS) using a Thermo TSQ Quantum Max triple quadrupole mass spectrometer. Table 16 and Table 17 and Table 18 provides the summary of the LC-MS/MS method.

TABLE 16
Summary of the LC-MS/MS method.

| | |
|---|---|
| Column | Accucore aQ column, 2.6 μm (100 × 2.1 mm) |
| Mobile Phase A | 99.9% H2O, 0.1% Formic Acid |
| Mobile Phase B | 99.9% ACN, 0.1% Formic Acid |
| Injection volume | 10 uL |

TABLE 17
HPLC Method

| Time (min) | Flow Rate (μL/min) | A % | B % |
|---|---|---|---|
| 0.00 | 500 | 95 | 5 |
| 1.00 | 500 | 95 | 5 |
| 1.50 | 500 | 10 | 90 |
| 3.50 | 500 | 10 | 90 |
| 3.51 | 500 | 95 | 5 |
| 4.00 | 500 | 95 | 5 |

TABLE 18
Tandem Mass Spectrometry

| | |
|---|---|
| Ion Source | HESI-II |
| Polarity | Positive |

SRM transitions

| | |
|---|---|
| Leucine | 132.1/30.5 |
| Isoleucine | 132.1/69.3 |
| Valine | 118.1/72.3 |
| Leucine-d₃ | 135.1/89.3 |

### Example 26. Lambda Red Recombination

Lambda red recombination is used to make chromosomal modifications, e.g., to delete leuE and/or ilvC, express one or more livKHMGF and/or leuDH(Bc)-kivD-adh2-brnQ-rrnB-ter cassette(s) or other cassettes described herein, e.g., driven by an FNR promoter or other promoter described herein, in E. coli Nissle. Lambda red is a procedure using recombination enzymes from a bacteriophage lambda to insert a piece of custom DNA into the chromosome of E. coli. A pKD46 plasmid is transformed into the E. coli Nissle host strain. E. coli Nissle cells are grown overnight in LB media. The overnight culture is diluted 1:100 in 5 mL of LB media and grown until it reaches an OD600 of 0.4-0.6. All tubes, solutions, and cuvettes are pre-chilled to 4° C. The E. coli cells are centrifuged at 2,000 rpm for 5 min at 4° C., the supernatant is removed, and the cells are resuspended in 1 mL of 4° C. water. The E. coli are centrifuged at 2,000 rpm for 5 min at 4° C., the supernatant is removed, and the cells are resuspended in 0.5 mL of 4° C. water. The E. coli are centrifuged at 2,000 rpm for 5 min at 4° C., the supernatant is removed, and the cells are resuspended in 0.1 mL of 4° C. water. The electroporator is set to 2.5 kV. 1 ng of pKD46 plasmid DNA is added to the E. coli cells, mixed by pipetting, and pipetted into a sterile, chilled cuvette. The dry cuvette is placed into the sample chamber, and the electric pulse is applied. 1 mL of room-temperature SOC media is immediately added, and the mixture is transferred to a culture tube and incubated at 30° C. for 1 hr. The cells are spread out on a selective media plate and incubated overnight at 30° C.

DNA sequences comprising the desired cassette(s) are ordered from a gene synthesis company. The lambda enzymes are used to insert this construct into the genome of E. coli Nissle through homologous recombination. The construct is inserted into a specific site in the genome of E. coli Nissle based on its DNA sequence. To insert the construct into a specific site, the homologous DNA sequence flanking the construct is identified, and includes approximately 50 bases on either side of the sequence. The homologous sequences are ordered as part of the synthesized gene. Alternatively, the homologous sequences may be added by PCR. The construct includes an antibiotic resistance marker that may be removed by recombination. The resulting construct comprises approximately 50 bases of homology upstream, a kanamycin resistance marker that can be removed by recombination, the cassette(s), and approximately 50 bases of homology downstream.

### Example 27. Generation of and Auxotrophy (ΔthyA)

An auxotrophic mutation causes bacteria to die in the absence of an exogenously added nutrient essential for survival or growth because they lack the gene(s) necessary to produce that essential nutrient. In order to generate genetically engineered bacteria with an auxotrophic modification in the genetically engineered strains, the thyA, a gene essential for oligonucleotide synthesis, is deleted. Deletion of the thyA gene in E. coli Nissle yields a strain that cannot form a colony on LB plates unless they are supplemented with thymidine.

A thyA::cam PCR fragment is amplified using 3 rounds of PCR as follows. Sequences of the primers used at a 100 um concentration are described in Table 19.

TABLE 19
Primer Sequences

| Name | Description | SEQ ID NO |
|---|---|---|
| SR36 | Round 1: binds on pKD3 | SEQ ID NO: 130 |
| SR38 | Round 1: binds on pKD3 | SEQ ID NO: 131 |
| SR33 | Round 2: binds to round 1 PCR product | SEQ ID NO: 132 |
| SR34 | Round 2: binds to round 1 PCR product | SEQ ID NO: 133 |
| SR43 | Round 3: binds to round 2 PCR product | SEQ ID NO: 134 |
| SR44 | Round 3: binds to round 2 PCR product | SEQ ID NO: 135 |

For the first PCR round, 4×50 ul PCR reactions containing ing pKD3 as template, 25 ul 2× phusion, 0.2 ul primer SR36 and SR38, and either 0, 0.2, 0.4 or 0.6 ul DMSO are brought up to 50 ul volume with nuclease free water and amplified under the following cycle conditions:

step1: 98 c for 30 s

step2: 98 c for 10 s

step3: 55 c for 15 s

step4: 72 c for 20 s

repeat step 2-4 for 30 cycles

step5: 72 c for 5 min

Subsequently, 5 ul of each PCR reaction is run on an agarose gel to confirm PCR product of the appropriate size. The PCR product is purified from the remaining PCR reaction using a Zymoclean gel DNA recovery kit according to the manufacturer's instructions and eluted in 30 ul nuclease free water.

For the second round of PCR, 1 ul purified PCR product from round 1 is used as template, in 4×50 ul PCR reactions as described above except with 0.2 ul of primers SR33 and SR34. Cycle conditions are the same as noted above for the first PCR reaction. The PCR product run on an agarose gel to verify amplification, purified, and eluted in 30 ul as described above.

For the third round of PCR, 1 ul of purified PCR product from round 2 is used as template in 4×50 ul PCR reactions as described except with primer SR43 and SR44. Cycle conditions are the same as described for rounds 1 and 2. Amplification is verified, the PCR product purified, and eluted as described above. The concentration and purity is measured using a spectrophotometer. The resulting linear DNA fragment, which contains 92 bp homologous to upstream of thyA, the chloramphenicol cassette flanked by frt sites, and 98 bp homologous to downstream of the thyA gene, is transformed into a E. coli Nissle 1917 strain containing pKD46 grown for recombineering. Following electroporation, 1 ml SOC medium containing 3 mM thymidine is added, and cells are allowed to recover at 37 C for 2 h with shaking. Cells are then pelleted at 10,000×g for 1 minute, the supernatant is discarded, and the cell pellet is resuspended in 100 ul LB containing 3 mM thymidine and spread on LB agar plates containing 3 mM thy and 20 ug/ml chloramphenicol. Cells are incubated at 37 C overnight. Colonies that appeared on LB plates are restreaked. +cam 20 ug/ml+ or −thy 3 mM. (thyA auxotrophs will only grow in media supplemented with thy 3 mM).

Next, the antibiotic resistance is removed with pCP20 transformation. pCP20 has the yeast Flp recombinase gene, FLP, chloramphenicol and ampicillin resistant genes, and temperature sensitive replication. Bacteria are grown in LB media containing the selecting antibiotic at 37° C. until OD600=0.4-0.6. 1 mL of cells are ished as follows: cells are pelleted at 16,000×g for 1 minute. The supernatant is discarded and the pellet is resuspended in 1 mL ice-cold 10% glycerol. This ish step is repeated 3× times. The final pellet is resuspended in 70 ul ice-cold 10% glycerol. Next, cells are electroporated with ing pCP20 plasmid DNA, and 1 mL SOC supplemented with 3 mM thymidine is immediately added to the cuvette. Cells are resuspended and transferred to a culture tube and grown at 30° C. for 1 hours. Cells are then pelleted at 10,000×g for 1 minute, the supernatant is discarded, and the cell pellet is resuspended in 100 ul LB containing 3 mM thymidine and spread on LB agar plates containing 3 mM thy and 100 ug/ml carbenicillin and grown at 30° C. for 16-24 hours. Next, transformants are colony purified non-selectively (no antibiotics) at 42° C.

To test the colony-purified transformants, a colony is picked from the 42° C. plate with a pipette tip and resuspended in 10μL LB. 3μL of the cell suspension is pipetted onto a set of 3 plates: Cam, (37° C.; tests for the presence/absence of CamR gene in the genome of the host strain), Amp, (30° C., tests for the presence/absence of AmpR from the pCP20 plasmid) and LB only (desired cells that have lost the chloramphenicol cassette and the pCP20 plasmid), 37° C. Colonies are considered cured if there is no growth in neither the Cam or Amp plate, picked, and re-streaked on an LB plate to get single colonies, and grown overnight at 37° C.

Subsequently, 5 ul of each PCR reaction is run on an agarose gel to confirm PCR product of the appropriate size. The PCR product is purified from the remaining PCR reaction using a Zymoclean gel DNA recovery kit according to the manufacturer's instructions and eluted in 30 ul nuclease free water.

For the second round of PCR, 1 ul purified PCR product from round 1 is used as template, in 4×50 ul PCR reactions as described above except with 0.2 ul of primers SR33 and SR34. Cycle conditions are the same as noted above for the first PCR reaction. The PCR product run on an agarose gel to verify amplification, purified, and eluted in 30 ul as described above.

For the third round of PCR, 1 ul of purified PCR product from round 2 is used as template in 4×50 ul PCR reactions as described except with primer SR43 and SR44. Cycle conditions are the same as described for rounds 1 and 2. Amplification is verified, the PCR product purified, and eluted as described above. The concentration and purity is measured using a spectrophotometer. The resulting linear DNA fragment, which contains 92 bp homologous to upstream of thyA, the chloramphenicol cassette flanked by frt sites, and 98 bp homologous to downstream of the thyA gene, is transformed into a E. coli Nissle 1917 strain containing pKD46 grown for recombineering. Following electroporation, 1 ml SOC medium containing 3 mM thymidine is added, and cells are allowed to recover at 37 C for 2 h with shaking. Cells are then pelleted at 10,000×g for 1 minute, the supernatant is discarded, and the cell pellet is resuspended in 100 ul LB containing 3 mM thymidine and spread on LB agar plates containing 3 mM thy and 20 ug/ml chloramphenicol. Cells are incubated at 37 C overnight. Colonies that appeared on LB plates are restreaked. +cam 20 ug/ml+ or −thy 3 mM. (thyA auxotrophs will only grow in media supplemented with thy 3 mM).

Next, the antibiotic resistance is removed with pCP20 transformation. pCP20 has the yeast Flp recombinase gene, FLP, chloramphenicol and ampicillin resistant genes, and temperature sensitive replication. Bacteria are grown in LB media containing the selecting antibiotic at 37° C. until OD600=0.4-0.6. 1 mL of cells are ished as follows: cells are pelleted at 16,000×g for 1 minute. The supernatant is discarded and the pellet is resuspended in 1 mL ice-cold 10% glycerol. This ish step is repeated 3× times. The final pellet is resuspended in 70 ul ice-cold 10% glycerol. Next, cells are electroporated with ing pCP20 plasmid DNA, and 1 mL SOC supplemented with 3 mM thymidine is immediately added to the cuvette. Cells are resuspended and transferred to a culture tube and grown at 30° C. for 1 hours. Cells are then pelleted at 10,000×g for 1 minute, the supernatant is discarded, and the cell pellet is resuspended in 100 ul LB containing 3 mM thymidine and spread on LB agar plates containing 3 mM thy and 100 ug/ml carbenicillin and grown at 30° C. for 16-24 hours. Next, transformants are colony purified non-selectively (no antibiotics) at 42° C.

To test the colony-purified transformants, a colony is picked from the 42° C. plate with a pipette tip and resuspended in 10μL LB. 3μL of the cell suspension is pipetted onto a set of 3 plates: Cam, (37° C.; tests for the presence/absence of CamR gene in the genome of the host strain), Amp, (30° C., tests for the presence/absence of AmpR from the pCP20 plasmid) and LB only (desired cells that have lost the chloramphenicol cassette and the pCP20 plasmid), 37° C. Colonies are considered cured if there is no growth in neither the Cam or Amp plate, picked, and re-streaked on an LB plate to get single colonies, and grown overnight at 37° C.

In other embodiments, similar methods are used to create other auxotrophies, including, but not limited to, dapA.

### Example 28. Nitric Oxide-Inducible Reporter Constructs

ATC and nitric oxide-inducible reporter constructs were synthesized (Genewiz, Cambridge, Mass.). When induced by their cognate inducers, these constructs express GFP, which is detected by monitoring fluorescence in a plate reader at an excitation/emission of 395/509 nm, respectively. Nissle cells harboring plasmids with either the control, ATC-inducible Ptet-GFP reporter construct, or the nitric oxide inducible PnsrR-GFP reporter construct were first grown to early log phase (OD600 of about 0.4-0.6), at which point they were transferred to 96-well microtiter plates containing LB and two-fold decreased inducer (ATC or the long half-life NO donor, DETA-NO (Sigma)). Both ATC and NO were able to induce the expression of GFP in their respective constructs across a range of concentrations (FIG. 83); promoter activity is expressed as relative florescence units. An exemplary sequence of a nitric oxide-inducible reporter construct is shown. The bsrR sequence is bolded. The gfp sequence is underlined. The PnsrR (NO regulated promoter and RBS) is italicized. The constitutive promoter and RBS are [symbol] . These constructs, when induced by their cognate inducer, lead to high level expression of GFP, which is detected by monitoring fluorescence in a plate reader at an excitation/emission of 395/509 nm, respectively. Nissle cells harboring plasmids with either the ATC-inducible Ptet-GFP reporter construct or the nitric oxide inducible PnsrR-GFP reporter construct were first grown to early log phase (OD600=˜0.4-0.6), at which point they were transferred to 96-well microtiter plates containing LB and 2-fold decreases in inducer (ATC or the long half-life NO donor, DETA-NO (Sigma)). It was observed that both the ATC and NO were able to induce the expression of GFP in their respective construct across a wide range of concentrations. Promoter activity is expressed as relative florescence units.

FIG. 83D shows a dot blot of NO-GFP constructs. E. coli Nissle harboring the nitric oxide inducible NsrR-GFP reporter fusion were grown overnight in LB supplemented with kanamycin. Bacteria were then diluted 1:100 into LB containing kanamycin and grown to an optical density of 0.4-0.5 and then pelleted by centrifugation. Bacteria were resuspended in phosphate buffered saline and 100 microliters were administered by oral gavage to mice. IBD is induced in mice by supplementing drinking water with 2-3% dextran sodium sulfate for 7 days prior to bacterial gavage. At 4 hours post-gavage, mice were sacrificed and bacteria were recovered from colonic samples. Colonic contents were boiled in SDS, and the soluble fractions were used to perform a dot blot for GFP detection (induction of NsrR-regulated promoters). Detection of GFP was performed by binding of anti-GFP antibody conjugated to HRP (horse radish peroxidase). Detection was visualized using Pierce chemiluminescent detection kit. It is shown in the figure that NsrR-regulated promoters are induced in DSS-treated mice, but are not shown to be induced in untreated mice. This is consistent with the role of NsrR in response to NO, and thus inflammation.

Bacteria harboring a plasmid expressing NsrR under control of a constitutive promoter and the reporter gene gfp (green fluorescent protein) under control of an NsrR-inducible promoter were grown overnight in LB supplemented with kanamycin. Bacteria are then diluted 1:100 into LB containing kanamycin and grown to an optical density of about 0.4-0.5 and then pelleted by centrifugation. Bacteria are resuspended in phosphate buffered saline and 100 microliters were administered by oral gavage to mice. IBD is induced in mice by supplementing drinking water with 2-3% dextran sodium sulfate for 7 days prior to bacterial gavage. At 4 hours post-gavage, mice were sacrificed and bacteria were recovered from colonic samples. Colonic contents were boiled in SDS, and the soluble fractions were used to perform a dot blot for GFP detection (induction of NsrR-regulated promoters) Detection of GFP was performed by binding of anti-GFP antibody conjugated to to HRP (horse radish peroxidase). Detection was visualized using Pierce chemiluminescent detection kit. FIG. 83D shows NsrR-regulated promoters are induced in DSS-treated mice, but not in untreated mice.

### Example 29. FNR Promoter Activity

In order to measure the promoter activity of different FNR promoters, the lacZ gene, as well as transcriptional and translational elements, were synthesized (Gen9, Cambridge, Mass.) and cloned into vector pBR322. The lacZ gene was placed under the control of any of the exemplary FNR promoter sequences disclosed in Table 3. The nucleotide sequences of these constructs are shown in Tables 20-24 (SEQ ID NO: 136-140). However, as noted above, the lacZ gene may be driven by other inducible promoters in order to analyze activities of those promoters, and other genes may be used in place of the lacZ gene as a readout for promoter activity, exemplary results are shown in FIG. 81.

Table 20 shows the nucleotide sequence of an exemplary construct comprising a gene encoding lacZ, and an exemplary FNR promoter, Pfnr1 (SEQ ID NO: 136). The construct comprises a translational fusion of the Nissle nirB1 gene and the lacZ gene, in which the translational fusions are fused in frame to the 8th codon of the lacZ coding region. The Pfnr1 sequence is bolded lower case, and the predicted ribosome binding site within the promoter is underlined. The lacZ sequence is underlined upper case. ATG site is bolded upper case, and the cloning sites used to synthesize the construct are shown in regular upper case.

Table 21 shows the nucleotide sequence of an exemplary construct comprising a gene encoding lacZ, and an exemplary FNR promoter, Pfnr2 (SEQ ID NO: 137). The construct comprises a translational fusion of the Nissle ydfZ gene and the lacZ gene, in which the translational fusions are fused in frame to the 8th codon of the lacZ coding region. The Pfnr2 sequence is bolded lower case, and the predicted ribosome binding site within the promoter is underlined. The lacZ sequence is underlined upper case. ATG site is bolded upper case, and the cloning sites used to synthesize the construct are shown in regular upper case.

Table 22 shows the nucleotide sequence of an exemplary construct comprising a gene encoding lacZ, and an exemplary FNR promoter, Pfn3 (SEQ ID NO: 138). The construct comprises a transcriptional fusion of the Nissle nirB gene and the lacZ gene, in which the transcriptional fusions use only the promoter region fused to a strong ribosomal binding site. The Pfn3 sequence is bolded lower case, and the predicted ribosome binding site within the promoter is underlined. The lacZ sequence is underlined upper case. ATG site is bolded upper case, and the cloning sites used to synthesize the construct are shown in regular upper case.

Table 23 shows the nucleotide sequence of an exemplary construct comprising a gene encoding lacZ, and an exemplary FNR promoter, Pfnr4 (SEQ ID NO: 139). The construct comprises a transcriptional fusion of the Nissle ydfZ gene and the lacZ gene. The Pfnr4 sequence is bolded lower case, and the predicted ribosome binding site within the promoter is underlined. The lacZ sequence is underlined upper case. ATG site is bolded upper case, and the cloning sites used to synthesize the construct are shown in regular upper case.

Table 24 shows the nucleotide sequence of an exemplary construct comprising a gene encoding lacZ, and an exemplary FNR promoter, PfnrS (SEQ ID NO: 140). The construct comprises a transcriptional fusion of the anaerobically induced small RNA gene, fnrS1, fused to lacZ. The Pfnrs sequence is bolded lower case, and the predicted ribosome binding site within the promoter is underlined. The lacZ sequence is underlined upper case. ATG site is bolded upper case, and the cloning sites used to synthesize the construct are shown in regular upper case.

TABLE 20
Pfnr1-lacZ Construct Sequences

Nucleotide sequences of Pfnr1-lacZ construct, low-
copy (SEQ ID NO: 136)
GGTACCgtcagcataacaccctgacctctcattaattgttcatgccgggc
ggcactatcgtcgtccggccttttcctctcttactctgctacgtacatct
atttctataaatccgttcaatttgtctgttttttgcacaaacatgaaata
tcagacaattccgtgacttaagaaaatttatacaaatcagcaatataccc
cttaaggagtatataaaggtgaatttgatttacatcaataagcggggttg
ctgaatcgttaaggtaggcggtaatagaaaagaaatcgaggcaaaaATGa
gcaaagtcagactcgcaattatGGATCCTCTGGCCGTCGTATTACAACGT
CGTGACTGGGAAAACCCTGGCGTTACCCAACTTAATCGCCTTGCGGCACA
TCCCCCTTTCGCCAGCTGGCGTAATAGCGAAGAGGCCCGCACCGATCGCC
CTTCCCAACAGTTGCGCAGCCTGAATGGCGAATGGCGCTTTGCCTGGTTT
CCGGCACCAGAAGCGGTGCCGGAAAGCTGGCTGGAGTGCGATCTTCCTGA
CGCCGATACTGTCGTCGTCCCCTCAAACTGGCAGATGCACGGTTACGATG
CGCCTATCTACACCAACGTGACCTATCCCATTACGGTCAATCCGCCGTTT
GTTCCCGCGGAGAATCCGACAGGTTGTTACTCGCTCACATTTAATATTGA
TGAAAGCTGGCTACAGGAAGGCCAGACGCGAATTATTTTTGATGGCGTTA
ACTCGGCGTTTCATCTGTGGTGCAACGGGCGCTGGGTCGGTTACGGCCAG
GACAGCCGTTTGCCGTCTGAATTTGACCTGAGCGCATTTTTACGCGCCGG
AGAAAACCGCCTCGCGGTGATGGTGCTGCGCTGGAGTGACGGCAGTTATC
TGGAAGATCAGGATATGTGGCGGATGAGCGGCATTTTCCGTGACGTCTCG
TTGCTGCATAAACCGACCACGCAAATCAGCGATTTCCAAGTTACCACTCT
CTTTAATGATGATTTCAGCCGCGCGGTACTGGAGGCAGAAGTTCAGATGT
ACGGCGAGCTGCGCGATGAACTGCGGGTGACGGTTTCTTTGTGGCAGGGT
GAAACGCAGGTCGCCAGCGGCACCGCGCCTTTCGGCGGTGAAATTATCGA
TGAGCGTGGCGGTTATGCCGATCGCGTCACACTACGCCTGAACGTTGAAA
ATCCGGAACTGTGGAGCGCCGAAATCCCGAATCTCTATCGTGCAGTGGTT
GAACTGCACACCGCCGACGGCACGCTGATTGAAGCAGAAGCCTGCGACGT
CGGTTTCCGCGAGGTGCGGATTGAAAATGGTCTGCTGCTGCTGAACGGCA
AGCCGTTGCTGATTCGCGGCGTTAACCGTCACGAGCATCATCCTCTGCAT
GGTCAGGTCATGGATGAGCAGACGATGGTGCAGGATATCCTGCTGATGAA
GCAGAACAACTTTAACGCCGTGCGCTGTTCGCATTATCCGAACCATCCGC
TGTGGTACACGCTGTGCGACCGCTACGGCCTGTATGTGGTGGATGAAGCC
Nucleotide sequences of Pfnr1-lacZ construct, low-
copy (SEQ ID NO: 136)
AATATTGAAACCCACGGCATGGTGCCAATGAATCGTCTGACCGATGATCC
GCGCTGGCTACCCGCGATGAGCGAACGCGTAACGCGGATGGTGCAGCGCG
ATCGTAATCACCCGAGTGTGATCATCTGGTCGCTGGGGAATGAATCAGGC
CACGGCGCTAATCACGACGCGCTGTATCGCTGGATCAAATCTGTCGATCC
TTCCCGCCCGGTACAGTATGAAGGCGGCGGAGCCGACACCACGGCCACCG
ATATTATTTGCCCGATGTACGCGCGCGTGGATGAAGACCAGCCCTTCCCG
GCGGTGCCGAAATGGTCCATCAAAAAATGGCTTTCGCTGCCTGGAGAAAT
GCGCCCGCTGATCCTTTGCGAATATGCCCACGCGATGGGTAACAGTCTTG
GCGGCTTCGCTAAATACTGGCAGGCGTTTCGTCAGTACCCCCGTTTACAG
GGCGGCTTCGTCTGGGACTGGGTGGATCAGTCGCTGATTAAATATGATGA
AAACGGCAACCCGTGGTCGGCTTACGGCGGTGATTTTGGCGATACGCCGA
ACGATCGCCAGTTCTGTATGAACGGTCTGGTCTTTGCCGACCGCACGCCG
CATCCGGCGCTGACGGAAGCAAAACACCAACAGCAGTATTTCCAGTTCCG
TTTATCCGGGCGAACCATCGAAGTGACCAGCGAATACCTGTTCCGTCATA
GCGATAACGAGTTCCTGCACTGGATGGTGGCACTGGATGGCAAGCCGCTG
GCAAGCGGTGAAGTGCCTCTGGATGTTGGCCCGCAAGGTAAGCAGTTGAT
TGAACTGCCTGAACTGCCGCAGCCGGAGAGCGCCGGACAACTCTGGCTAA
CGGTACGCGTAGTGCAACCAAACGCGACCGCATGGTCAGAAGCCGGACAC
ATCAGCGCCTGGCAGCAATGGCGTCTGGCGGAAAACCTCAGCGTGACACT
CCCCTCCGCGTCCCACGCCATCCCTCAACTGACCACCAGCGGAACGGATT
TTTGCATCGAGCTGGGTAATAAGCGTTGGCAATTTAACCGCCAGTCAGGC
TTTCTTTCACAGATGTGGATTGGCGATGAAAAACAACTGCTGACCCCGCT
GCGCGATCAGTTCACCCGTGCGCCGCTGGATAACGACATTGGCGTAAGTG
AAGCGACCCGCATTGACCCTAACGCCTGGGTCGAACGCTGGAAGGCGGCG
GGCCATTACCAGGCCGAAGCGGCGTTGTTGCAGTGCACGGCAGATACACT
TGCCGACGCGGTGCTGATTACAACCGCCCACGCGTGGCAGCATCAGGGGA
AAACCTTATTTATCAGCCGGAAAACCTACCGGATTGATGGGCACGGTGAG
ATGGTCATCAATGTGGATGTTGCGGTGGCAAGCGATACACCGCATCCGGC
GCGGATTGGCCTGACCTGCCAGCTGGCGCAGGTCTCAGAGCGGGTAAACT
GGCTCGGCCTGGGGCCGCAAGAAAACTATCCCGACCGCCTTACTGCAGCC
TGTTTTGACCGCTGGGATCTGCCATTGTCAGACATGTATACCCCGTACGT
CTTCCCGAGCGAAAACGGTCTGCGCTGCGGGACGCGCGAATTGAATTATG
GCCCACACCAGTGGCGCGGCGACTTCCAGTTCAACATCAGCCGCTACAGC
CAACAACAACTGATGGAAACCAGCCATCGCCATCTGCTGCACGCGGAAGA
AGGCACATGGCTGAATATCGACGGTTTCCATATGGGGATTGGTGGCGACG
ACTCCTGGAGCCCGTCAGTATCGGCGGAATTCCAGCTGAGCGCCGGTCGC
TACCATTACCAGTTGGTCTGGTGTCAAAAATAA

TABLE 21
Pfnr2-lacZ Construct Sequences

Nucleotide sequences of Pfnr2-lacZ construct,
low-copy (SEQ ID NO: 137)
GGTACCcatttcctctcatcccatccggggtgagagtcttttcccccgac
ttatggctcatgcatgcatcaaaaaagatgtgagcttgatcaaaaacaaa
aaatatttcactcgacaggagtatttatattgcgcccgttacgtgggctt
cgactgtaaatcagaaaggagaaaacacctATGacgacctacgatcgGGA
TCCTCTGGCCGTCGTATTACAACGTCGTGACTGGGAAAACCCTGGCGTTA
CCCAACTTAATCGCCTTGCGGCACATCCCCCTTTCGCCAGCTGGCGTAAT
AGCGAAGAGGCCCGCACCGATCGCCCTTCCCAACAGTTGCGCAGCCTGAA
TGGCGAATGGCGCTTTGCCTGGTTTCCGGCACCAGAAGCGGTGCCGGAAA
GCTGGCTGGAGTGCGATCTTCCTGACGCCGATACTGTCGTCGTCCCCTCA
AACTGGCAGATGCACGGTTACGATGCGCCTATCTACACCAACGTGACCTA
TCCCATTACGGTCAATCCGCCGTTTGTTCCCGCGGAGAATCCGACAGGTT
GTTACTCGCTCACATTTAATATTGATGAAAGCTGGCTACAGGAAGGCCAG
ACGCGAATTATTTTTGATGGCGTTAACTCGGCGTTTCATCTGTGGTGCAA
CGGGCGCTGGGTCGGTTACGGCCAGGACAGCCGTTTGCCGTCTGAATTTG
ACCTGAGCGCATTTTTACGCGCCGGAGAAAACCGCCTCGCGGTGATGGTG
CTGCGCTGGAGTGACGGCAGTTATCTGGAAGATCAGGATATGTGGCGGAT
GAGCGG
Nucleotide sequences of Pfnr2-lacZ construct,
low-copy (SEQ ID NO: 137)
CATTTTCCGTGACGTCTCGTTGCTGCATAAACCGACCACGCAAATCAGCG
ATTTCCAAGTTACCACTCTCTTTAATGATGATTTCAGCCGCGCGGTACTG
GAGGCAGAAGTTCAGATGTACGGCGAGCTGCGCGATGAACTGCGGGTGAC
GGTTTCTTTGTGGCAGGGTGAAACGCAGGTCGCCAGCGGCACCGCGCCTT
TCGGCGGTGAAATTATCGATGAGCGTGGCGGTTATGCCGATCGCGTCACA
CTACGCCTGAACGTTGAAAATCCGGAACTGTGGAGCGCCGAAATCCCGAA
TCTCTATCGTGCAGTGGTTGAACTGCACACCGCCGACGGCACGCTGATTG
AAGCAGAAGCCTGCGACGTCGGTTTCCGCGAGGTGCGGATTGAAAATGGT
CTGCTGCTGCTGAACGGCAAGCCGTTGCTGATTCGCGGCGTTAACCGTCA
CGAGCATCATCCTCTGCATGGTCAGGTCATGGATGAGCAGACGATGGTGC
AGGATATCCTGCTGATGAAGCAGAACAACTTTAACGCCGTGCGCTGTTCG
CATTATCCGAACCATCCGCTGTGGTACACGCTGTGCGACCGCTACGGCCT
GTATGTGGTGGATGAAGCCAATATTGAAACCCACGGCATGGTGCCAATGA
ATCGTCTGACCGATGATCCGCGCTGGCTACCCGCGATGAGCGAACGCGTA
ACGCGGATGGTGCAGCGCGATCGTAATCACCCGAGTGTGATCATCTGGTC
GCTGGGGAATGAATCAGGCCACGGCGCTAATCACGACGCGCTGTATCGCT
GGATCAAATCTGTCGATCCTTCCCGCCCGGTACAGTATGAAGGCGGCGGA
GCCGACACCACGGCCACCGATATTATTTGCCCGATGTACGCGCGCGTGGA
TGAAGACCAGCCCTTCCCGGCGGTGCCGAAATGGTCCATCAAAAAATGGC
TTTCGCTGCCTGGAGAAATGCGCCCGCTGATCCTTTGCGAATATGCCCAC
GCGATGGGTAACAGTCTTGGCGGCTTCGCTAAATACTGGCAGGCGTTTCG
TCAGTACCCCCGTTTACAGGGCGGCTTCGTCTGGGACTGGGTGGATCAGT
CGCTGATTAAATATGATGAAAACGGCAACCCGTGGTCGGCTTACGGCGGT
GATTTTGGCGATACGCCGAACGATCGCCAGTTCTGTATGAACGGTCTGGT
CTTTGCCGACCGCACGCCGCATCCGGCGCTGACGGAAGCAAAACACCAAC
AGCAGTATTTCCAGTTCCGTTTATCCGGGCGAACCATCGAAGTGACCAGC
GAATACCTGTTCCGTCATAGCGATAACGAGTTCCTGCACTGGATGGTGGC
ACTGGATGGCAAGCCGCTGGCAAGCGGTGAAGTGCCTCTGGATGTTGGCC
CGCAAGGTAAGCAGTTGATTGAACTGCCTGAACTGCCGCAGCCGGAGAGC
GCCGGACAACTCTGGCTAACGGTACGCGTAGTGCAACCAAACGCGACCGC
ATGGTCAGAAGCCGGACACATCAGCGCCTGGCAGCAATGGCGTCTGGCGG
AAAACCTCAGCGTGACACTCCCCTCCGCGTCCCACGCCATCCCTCAACTG
ACCACCAGCGGAACGGATTTTTGCATCGAGCTGGGTAATAAGCGTTGGCA
ATTTAACCGCCAGTCAGGCTTTCTTTCACAGATGTGGATTGGCGATGAAA
AACAACTGCTGACCCCGCTGCGCGATCAGTTCACCCGTGCGCCGCTGGAT
AACGACATTGGCGTAAGTGAAGCGACCCGCATTGACCCTAACGCCTGGGT
CGAACGCTGGAAGGCGGCGGGCCATTACCAGGCCGAAGCGGCGTTGTTGC
AGTGCACGGCAGATACACTTGCCGACGCGGTGCTGATTACAACCGCCCAC
GCGTGGCAGCATCAGGGGAAAACCTTATTTATCAGCCGGAAAACCTACCG
GATTGATGGGCACGGTGAGATGGTCATCAATGTGGATGTTGCGGTGGCAA
GCGATACACCGCATCCGGCGCGGATTGGCCTGACCTGCCAGCTGGCGCAG
GTCTCAGAGCGGGTAAACTGGCTCGGCCTGGGGCCGCAAGAAAACTATCC
CGACCGCCTTACTGCAGCCTGTTTTGACCGCTGGGATCTGCCATTGTCAG
ACATGTATACCCCGTACGTCTTCCCGAGCGAAAACGGTCTGCGCTGCGGG
ACGCGCGAATTGAATTATGGCCCACACCAGTGGCGCGGCGACTTCCAGTT
CAACATCAGCCGCTACAGCCAACAACAACTGATGGAAACCAGCCATCGCC
ATCTGCTGCACGCGGAAGAAGGCACATGGCTGAATATCGACGGTTTCCAT
ATGGGGATTGGTGGCGACGACTCCTGGAGCCCGTCAGTATCGGCGGAATT
CCAGCTGAGCGCCGGTCGCTACCATTACCAGTTGGTCTGGTGTCAAAAAT
AA

TABLE 22
Pfnr3-lacZ Construct Sequences

Nucleotide sequences of Pfnr3-lacZ construct,
low-copy (SEQ ID NO: 138)
GGTACCgtcagcataacaccctgacctctcattaattgttcatgccgggc
ggcactatcgtcgtccggccttttcctctcttactctgctacgtacatct
atttctataaatccgttcaatttg
Nucleotide sequences of Pfnr3-lacZ construct,
low-copy (SEQ ID NO: 138)
tctgttttttgcacaaacatgaaatatcagacaattccgtgacttaagaa
aatttatacaaatcagcaatataccccttaaggagtatataaaggtgaat
ttgatttacatcaataagcggggttgctgaatcgttaaGGATCCctctag
aaataattttgtttaactttaagaaggagatatacatATGACTATGATTA
CGGATTCTCTGGCCGTCGTATTACAACGTCGTGACTGGGAAAACCCTGGC
GTTACCCAACTTAATCGCCTTGCGGCACATCCCCCTTTCGCCAGCTGGCG
TAATAGCGAAGAGGCCCGCACCGATCGCCCTTCCCAACAGTTGCGCAGCC
TGAATGGCGAATGGCGCTTTGCCTGGTTTCCGGCACCAGAAGCGGTGCCG
GAAAGCTGGCTGGAGTGCGATCTTCCTGACGCCGATACTGTCGTCGTCCC
CTCAAACTGGCAGATGCACGGTTACGATGCGCCTATCTACACCAACGTGA
CCTATCCCATTACGGTCAATCCGCCGTTTGTTCCCGCGGAGAATCCGACA
GGTTGTTACTCGCTCACATTTAATATTGATGAAAGCTGGCTACAGGAAGG
CCAGACGCGAATTATTTTTGATGGCGTTAACTCGGCGTTTCATCTGTGGT
GCAACGGGCGCTGGGTCGGTTACGGCCAGGACAGCCGTTTGCCGTCTGAA
TTTGACCTGAGCGCATTTTTACGCGCCGGAGAAAACCGCCTCGCGGTGAT
GGTGCTGCGCTGGAGTGACGGCAGTTATCTGGAAGATCAGGATATGTGGC
GGATGAGCGGCATTTTCCGTGACGTCTCGTTGCTGCATAAACCGACCACG
CAAATCAGCGATTTCCAAGTTACCACTCTCTTTAATGATGATTTCAGCCG
CGCGGTACTGGAGGCAGAAGTTCAGATGTACGGCGAGCTGCGCGATGAAC
TGCGGGTGACGGTTTCTTTGTGGCAGGGTGAAACGCAGGTCGCCAGCGGC
ACCGCGCCTTTCGGCGGTGAAATTATCGATGAGCGTGGCGGTTATGCCGA
TCGCGTCACACTACGCCTGAACGTTGAAAATCCGGAACTGTGGAGCGCCG
AAATCCCGAATCTCTATCGTGCAGTGGTTGAACTGCACACCGCCGACGGC
ACGCTGATTGAAGCAGAAGCCTGCGACGTCGGTTTCCGCGAGGTGCGGAT
TGAAAATGGTCTGCTGCTGCTGAACGGCAAGCCGTTGCTGATTCGCGGCG
TTAACCGTCACGAGCATCATCCTCTGCATGGTCAGGTCATGGATGAGCAG
ACGATGGTGCAGGATATCCTGCTGATGAAGCAGAACAACTTTAACGCCGT
GCGCTGTTCGCATTATCCGAACCATCCGCTGTGGTACACGCTGTGCGACC
GCTACGGCCTGTATGTGGTGGATGAAGCCAATATTGAAACCCACGGCATG
GTGCCAATGAATCGTCTGACCGATGATCCGCGCTGGCTACCCGCGATGAG
CGAACGCGTAACGCGGATGGTGCAGCGCGATCGTAATCACCCGAGTGTGA
TCATCTGGTCGCTGGGGAATGAATCAGGCCACGGCGCTAATCACGACGCG
CTGTATCGCTGGATCAAATCTGTCGATCCTTCCCGCCCGGTACAGTATGA
AGGCGGCGGAGCCGACACCACGGCCACCGATATTATTTGCCCGATGTACG
CGCGCGTGGATGAAGACCAGCCCTTCCCGGCGGTGCCGAAATGGTCCATC
AAAAAATGGCTTTCGCTGCCTGGAGAAATGCGCCCGCTGATCCTTTGCGA
ATATGCCCACGCGATGGGTAACAGTCTTGGCGGCTTCGCTAAATACTGGC
AGGCGTTTCGTCAGTACCCCCGTTTACAGGGCGGCTTCGTCTGGGACTGG
GTGGATCAGTCGCTGATTAAATATGATGAAAACGGCAACCCGTGGTCGGC
TTACGGCGGTGATTTTGGCGATACGCCGAACGATCGCCAGTTCTGTATGA
ACGGTCTGGTCTTTGCCGACCGCACGCCGCATCCGGCGCTGACGGAAGCA
AAACACCAACAGCAGTATTTCCAGTTCCGTTTATCCGGGCGAACCATCGA
AGTGACCAGCGAATACCTGTTCCGTCATAGCGATAACGAGTTCCTGCACT
GGATGGTGGCACTGGATGGCAAGCCGCTGGCAAGCGGTGAAGTGCCTCTG
GATGTTGGCCCGCAAGGTAAGCAGTTGATTGAACTGCCTGAACTGCCGCA
GCCGGAGAGCGCCGGACAACTCTGGCTAACGGTACGCGTAGTGCAACCAA
ACGCGACCGCATGGTCAGAAGCCGGACACATCAGCGCCTGGCAGCAATGG
CGTCTGGCGGAAAACCTCAGCGTGACACTCCCCTCCGCGTCCCACGCCAT
CCCTCAACTGACCACCAGCGGAACGGATTTTTGCATCGAGCTGGGTAATA
AGCGTTGGCAATTTAACCGCCAGTCAGGCTTTCTTTCACAGATGTGGATT
GGCGATGAAAAACAACTGCTGACCCCGCTGCGCGATCAGTTCACCCGTGC
GCCGCTGGATAACGACATTGGCGTAAGTGAAGCGACCCGCATTGACCCTA
ACGCCTGGGTCGAACGCTGGAAGGCGGCGGGCCATTACCAGGCCGAAGCG
GCGTTGTTGCAGTGCACGGCAGATACACTTGCCGACGCGGTGCTGATTAC
AACCGCCCACGCGTGGCAGCATCAGGGGAAAACCTTATTTATCAGCCGGA
AAACCTACCGGATTGATGGGCACGGTGAGATGGTCATCAATGTGGATGTT
GCGGTGGCAAGCGATACACCGCATCCGGCGCGGATTGGCCTGACCTGCCA
GCTGGCGCAGGTCTCAGAGCGGGTAAACTGGCTCGGCCTGGGGCCGCAAG
AAAACTATCCCGACCGCCTTACTGCAGCCTGTTTTGACCGCTGGGATCTG
CCATTGTCAGACATGTATACCCCGTACGTCTTCCCGAGCGAAAACGGTCT
GCGCTGCGGGACGCGCGAATTGAATTATGGCCCACACC
Nucleotide sequences of Pfnr3-lacZ construct,
low-copy (SEQ ID NO: 138)
AGTGGCGCGGCGACTTCCAGTTCAACATCAGCCGCTACAGCCAACAACAA
CTGATGGAAACCAGCCATCGCCATCTGCTGCACGCGGAAGAAGGCACATG
GCTGAATATCGACGGTTTCCATATGGGGATTGGTGGCGACGACTCCTGGA
GCCCGTCAGTATCGGCGGAATTCCAGCTGAGCGCCGGTCGCTACCATTAC
CAGTTGGTCTGGTGTCAAAAATAA

TABLE 23
Pfnr4-lacZ construct Sequences

Nucleotide sequences of Pfnr4-lacZ construct,
low-copy (SEQ ID NO: 139)
GGTACCcatttcctctcatcccatccggggtgagagtcttttcccccgac
ttatggctcatgcatgcatcaaaaaagatgtgagcttgatcaaaaacaaa
aaatatttcactcgacaggagtatttatattgcgcccGGATCCctctaga
aataattttgtttaactttaagaaggagatatacatATGACTATGATTAC
GGATTCTCTGGCCGTCGTATTACAACGTCGTGACTGGGAAAACCCTGGCG
TTACCCAACTTAATCGCCTTGCGGCACATCCCCCTTTCGCCAGCTGGCGT
AATAGCGAAGAGGCCCGCACCGATCGCCCTTCCCAACAGTTGCGCAGCCT
GAATGGCGAATGGCGCTTTGCCTGGTTTCCGGCACCAGAAGCGGTGCCGG
AAAGCTGGCTGGAGTGCGATCTTCCTGACGCCGATACTGTCGTCGTCCCC
TCAAACTGGCAGATGCACGGTTACGATGCGCCTATCTACACCAACGTGAC
CTATCCCATTACGGTCAATCCGCCGTTTGTTCCCGCGGAGAATCCGACAG
GTTGTTACTCGCTCACATTTAATATTGATGAAAGCTGGCTACAGGAAGGC
CAGACGCGAATTATTTTTGATGGCGTTAACTCGGCGTTTCATCTGTGGTG
CAACGGGCGCTGGGTCGGTTACGGCCAGGACAGCCGTTTGCCGTCTGAAT
TTGACCTGAGCGCATTTTTACGCGCCGGAGAAAACCGCCTCGCGGTGATG
GTGCTGCGCTGGAGTGACGGCAGTTATCTGGAAGATCAGGATATGTGGCG
GATGAGCGGCATTTTCCGTGACGTCTCGTTGCTGCATAAACCGACCACGC
AAATCAGCGATTTCCAAGTTACCACTCTCTTTAATGATGATTTCAGCCGC
GCGGTACTGGAGGCAGAAGTTCAGATGTACGGCGAGCTGCGCGATGAACT
GCGGGTGACGGTTTCTTTGTGGCAGGGTGAAACGCAGGTCGCCAGCGGCA
CCGCGCCTTTCGGCGGTGAAATTATCGATGAGCGTGGCGGTTATGCCGAT
CGCGTCACACTACGCCTGAACGTTGAAAATCCGGAACTGTGGAGCGCCGA
AATCCCGAATCTCTATCGTGCAGTGGTTGAACTGCACACCGCCGACGGCA
CGCTGATTGAAGCAGAAGCCTGCGACGTCGGTTTCCGCGAGGTGCGGATT
GAAAATGGTCTGCTGCTGCTGAACGGCAAGCCGTTGCTGATTCGCGGCGT
TAACCGTCACGAGCATCATCCTCTGCATGGTCAGGTCATGGATGAGCAGA
CGATGGTGCAGGATATCCTGCTGATGAAGCAGAACAACTTTAACGCCGTG
CGCTGTTCGCATTATCCGAACCATCCGCTGTGGTACACGCTGTGCGACCG
CTACGGCCTGTATGTGGTGGATGAAGCCAATATTGAAACCCACGGCATGG
TGCCAATGAATCGTCTGACCGATGATCCGCGCTGGCTACCCGCGATGAGC
GAACGCGTAACGCGGATGGTGCAGCGCGATCGTAATCACCCGAGTGTGAT
CATCTGGTCGCTGGGGAATGAATCAGGCCACGGCGCTAATCACGACGCGC
TGTATCGCTGGATCAAATCTGTCGATCCTTCCCGCCCGGTACAGTATGAA
GGCGGCGGAGCCGACACCACGGCCACCGATATTATTTGCCCGATGTACGC
GCGCGTGGATGAAGACCAGCCCTTCCCGGCGGTGCCGAAATGGTCCATCA
AAAAATGGCTTTCGCTGCCTGGAGAAATGCGCCCGCTGATCCTTTGCGAA
TATGCCCACGCGATGGGTAACAGTCTTGGCGGCTTCGCTAAATACTGGCA
GGCGTTTCGTCAGTACCCCCGTTTACAGGGCGGCTTCGTCTGGGACTGGG
TGGATCAGTCGCTGATTAAATATGATGAAAACGGCAACCCGTGGTCGGCT
TACGGCGGTGATTTTGGCGATACGCCGAACGATCGCCAGTTCTGTATGAA
CGGTCTGGTCTTTGCCGACCGCACGCCGCATCCGGCGCTGACGGAAGCAA
AACACCAACAGCAGTATTTCCAGTTCCGTTTATCCGGGCGAACCATCGAA
GTGACCAGCGAATACCTGTTCCGTCATAGCGATAACGAGTTCCTGCACTG
GATGGTGGCACTGGATGGCA
Nucleotide sequences of Pfnr4-lacZ construct,
low-copy (SEQ ID NO: 139)
AGCCGCTGGCAAGCGGTGAAGTGCCTCTGGATGTTGGCCCGCAAGGTAAG
CAGTTGATTGAACTGCCTGAACTGCCGCAGCCGGAGAGCGCCGGACAACT
CTGGCTAACGGTACGCGTAGTGCAACCAAACGCGACCGCATGGTCAGAAG
CCGGACACATCAGCGCCTGGCAGCAATGGCGTCTGGCGGAAAACCTCAGC
GTGACACTCCCCTCCGCGTCCCACGCCATCCCTCAACTGACCACCAGCGG
AACGGATTTTTGCATCGAGCTGGGTAATAAGCGTTGGCAATTTAACCGCC
AGTCAGGCTTTCTTTCACAGATGTGGATTGGCGATGAAAAACAACTGCTG
ACCCCGCTGCGCGATCAGTTCACCCGTGCGCCGCTGGATAACGACATTGG
CGTAAGTGAAGCGACCCGCATTGACCCTAACGCCTGGGTCGAACGCTGGA
AGGCGGCGGGCCATTACCAGGCCGAAGCGGCGTTGTTGCAGTGCACGGCA
GATACACTTGCCGACGCGGTGCTGATTACAACCGCCCACGCGTGGCAGCA
TCAGGGGAAAACCTTATTTATCAGCCGGAAAACCTACCGGATTGATGGGC
ACGGTGAGATGGTCATCAATGTGGATGTTGCGGTGGCAAGCGATACACCG
CATCCGGCGCGGATTGGCCTGACCTGCCAGCTGGCGCAGGTCTCAGAGCG
GGTAAACTGGCTCGGCCTGGGGCCGCAAGAAAACTATCCCGACCGCCTTA
CTGCAGCCTGTTTTGACCGCTGGGATCTGCCATTGTCAGACATGTATACC
CCGTACGTCTTCCCGAGCGAAAACGGTCTGCGCTGCGGGACGCGCGAATT
GAATTATGGCCCACACCAGTGGCGCGGCGACTTCCAGTTCAACATCAGCC
GCTACAGCCAACAACAACTGATGGAAACCAGCCATCGCCATCTGCTGCAC
GCGGAAGAAGGCACATGGCTGAATATCGACGGTTTCCATATGGGGATTGG
TGGCGACGACTCCTGGAGCCCGTCAGTATCGGCGGAATTCCAGCTGAGCG
CCGGTCGCTACCATTACCAGTTGGTCTGGTGTCAAAAATAA

TABLE 24
Pfnrs-lacZ Construct Sequences

Nucleotide sequences of Pfnrs-lacZ construct,
low-copy (SEQ ID NO: 140)
GGTACCagttgttcttattggtggtgttgctttatggttgcatcgtagta
aatggttgtaacaaaagcaatttttccggctgtctgtatacaaaaacgcc
gtaaagtttgagcgaagtcaataaactctctacccattcagggcaatatc
tctcttGGATCCctctagaaataattttgtttaactttaagaaggagata
tacatATGCTATGATTACGGATTCTCTGGCCGTCGTATTACAACGTCGTG
ACTGGGAAAACCCTGGCGTTACCCAACTTAATCGCCTTGCGGCACATCCC
CCTTTCGCCAGCTGGCGTAATAGCGAAGAGGCCCGCACCGATCGCCCTTC
CCAACAGTTGCGCAGCCTGAATGGCGAATGGCGCTTTGCCTGGTTTCCGG
CACCAGAAGCGGTGCCGGAAAGCTGGCTGGAGTGCGATCTTCCTGACGCC
GATACTGTCGTCGTCCCCTCAAACTGGCAGATGCACGGTTACGATGCGCC
TATCTACACCAACGTGACCTATCCCATTACGGTCAATCCGCCGTTTGTTC
CCGCGGAGAATCCGACAGGTTGTTACTCGCTCACATTTAATATTGATGAA
AGCTGGCTACAGGAAGGCCAGACGCGAATTATTTTTGATGGCGTTAACTC
GGCGTTTCATCTGTGGTGCAACGGGCGCTGGGTCGGTTACGGCCAGGACA
GCCGTTTGCCGTCTGAATTTGACCTGAGCGCATTTTTACGCGCCGGAGAA
AACCGCCTCGCGGTGATGGTGCTGCGCTGGAGTGACGGCAGTTATCTGGA
AGATCAGGATATGTGGCGGATGAGCGGCATTTTCCGTGACGTCTCGTTGC
TGCATAAACCGACCACGCAAATCAGCGATTTCCAAGTTACCACTCTCTTT
AATGATGATTTCAGCCGCGCGGTACTGGAGGCAGAAGTTCAGATGTACGG
CGAGCTGCGCGATGAACTGCGGGTGACGGTTTCTTTGTGGCAGGGTGAAA
CGCAGGTCGCCAGCGGCACCGCGCCTTTCGGCGGTGAAATTATCGATGAG
CGTGGCGGTTATGCCGATCGCGTCACACTACGCCTGAACGTTGAAAATCC
GGAACTGTGGAGCGCCGAAATCCCGAATCTCTATCGTGCAGTGGTTGAAC
TGCACACCGCCGACGGCACGCTGATTGAAGCAGAAGCCTGCGACGTCGGT
TTCCGCGAGGTGCGGATTGAAAATGGTCTGCTGCTGCTGAACGGCAAGCC
GTTGCTGATTCGCGGCGTTAACCGTCACGAGCATCATCCTCTGCATGGTC
AGGTCATGGATGAGCAGACGATGGTGCAGGATATCCTGCTGATGAAGCAG
AACAACTTTAACGCCGTGCGCTGTTCGCATTATCCGAACCATCCGCTGTG
GTACACGCTGTGCGACCGCTACGGCC
Nucleotide sequences of Pfnrs-lacZ construct,
low-copy (SEQ ID NO: 140)
TGTATGTGGTGGATGAAGCCAATATTGAAACCCACGGCATGGTGCCAATG
AATCGTCTGACCGATGATCCGCGCTGGCTACCCGCGATGAGCGAACGCGT
AACGCGGATGGTGCAGCGCGATCGTAATCACCCGAGTGTGATCATCTGGT
CGCTGGGGAATGAATCAGGCCACGGCGCTAATCACGACGCGCTGTATCGC
TGGATCAAATCTGTCGATCCTTCCCGCCCGGTACAGTATGAAGGCGGCGG
AGCCGACACCACGGCCACCGATATTATTTGCCCGATGTACGCGCGCGTGG
ATGAAGACCAGCCCTTCCCGGCGGTGCCGAAATGGTCCATCAAAAAATGG
CTTTCGCTGCCTGGAGAAATGCGCCCGCTGATCCTTTGCGAATATGCCCA
CGCGATGGGTAACAGTCTTGGCGGCTTCGCTAAATACTGGCAGGCGTTTC
GTCAGTACCCCCGTTTACAGGGCGGCTTCGTCTGGGACTGGGTGGATCAG
TCGCTGATTAAATATGATGAAAACGGCAACCCGTGGTCGGCTTACGGCGG
TGATTTTGGCGATACGCCGAACGATCGCCAGTTCTGTATGAACGGTCTGG
TCTTTGCCGACCGCACGCCGCATCCGGCGCTGACGGAAGCAAAACACCAA
CAGCAGTATTTCCAGTTCCGTTTATCCGGGCGAACCATCGAAGTGACCAG
CGAATACCTGTTCCGTCATAGCGATAACGAGTTCCTGCACTGGATGGTGG
CACTGGATGGCAAGCCGCTGGCAAGCGGTGAAGTGCCTCTGGATGTTGGC
CCGCAAGGTAAGCAGTTGATTGAACTGCCTGAACTGCCGCAGCCGGAGAG
CGCCGGACAACTCTGGCTAACGGTACGCGTAGTGCAACCAAACGCGACCG
CATGGTCAGAAGCCGGACACATCAGCGCCTGGCAGCAATGGCGTCTGGCG
GAAAACCTCAGCGTGACACTCCCCTCCGCGTCCCACGCCATCCCTCAACT
GACCACCAGCGGAACGGATTTTTGCATCGAGCTGGGTAATAAGCGTTGGC
AATTTAACCGCCAGTCAGGCTTTCTTTCACAGATGTGGATTGGCGATGAA
AAACAACTGCTGACCCCGCTGCGCGATCAGTTCACCCGTGCGCCGCTGGA
TAACGACATTGGCGTAAGTGAAGCGACCCGCATTGACCCTAACGCCTGGG
TCGAACGCTGGAAGGCGGCGGGCCATTACCAGGCCGAAGCGGCGTTGTTG
CAGTGCACGGCAGATACACTTGCCGACGCGGTGCTGATTACAACCGCCCA
CGCGTGGCAGCATCAGGGGAAAACCTTATTTATCAGCCGGAAAACCTACC
GGATTGATGGGCACGGTGAGATGGTCATCAATGTGGATGTTGCGGTGGCA
AGCGATACACCGCATCCGGCGCGGATTGGCCTGACCTGCCAGCTGGCGCA
GGTCTCAGAGCGGGTAAACTGGCTCGGCCTGGGGCCGCAAGAAAACTATC
CCGACCGCCTTACTGCAGCCTGTTTTGACCGCTGGGATCTGCCATTGTCA
GACATGTATACCCCGTACGTCTTCCCGAGCGAAAACGGTCTGCGCTGCGG
GACGCGCGAATTGAATTATGGCCCACACCAGTGGCGCGGCGACTTCCAGT
TCAACATCAGCCGCTACAGCCAACAACAACTGATGGAAACCAGCCATCGC
CATCTGCTGCACGCGGAAGAAGGCACATGGCTGAATATCGACGGTTTCCA
TATGGGGATTGGTGGCGACGACTCCTGGAGCCCGTCAGTATCGGCGGAAT
TCCAGCTGAGCGCCGGTCGCTACCATTACCAGTTGGTCTGGTGTCAAAAA
TAA

### Example 30. Table of Sequences

TABLE 25
Table of Sequences

| SEQ ID NO | Description |
|---|---|
| SEQ ID NO: 14 | FNR responsive regulatory sequence |
| SEQ ID NO: 15 | FNR responsive regulatory sequence |
| SEQ ID NO: 16 | FNR responsive regulatory sequence |
| SEQ ID NO: 17 | FNR responsive regulatory sequence |
| SEQ ID NO: 18 | FNR responsive regulatory sequence |
| SEQ ID NO: 80 | SEQ ID NO; FNR responsive regulatory sequence |
| SEQ ID NO: 81 | SEQ ID NO; FNR responsive regulatory sequence |
| SEQ ID NO: 82 | nirB1 |
| SEQ ID NO: 83 | nirB2 |
| SEQ ID NO: 84 | nirB3 |
| SEQ ID NO: 85 | ydfZ |
| SEQ ID NO: 86 | nirB + RBS |
| SEQ ID NO: 87 | ydfZ + RBS |
| SEQ ID NO: 88 | fnrS1 |
| SEQ ID NO: 89 | fnrS2 |
| SEQ ID NO: 90 | nirB + crp |
| SEQ ID NO: 143 | fnrS + crp |
| SEQ ID NO: 46 | katG |
| SEQ ID NO: 47 | dps |
| SEQ ID NO: 48 | ahpC |
| SEQ ID NO: 49 | oxyS |
| SEQ ID NO: 1 | kivD gene from Lactococcus lactis IFPL730 |
| SEQ ID NO: 2 | Tet-kivD construct |
| SEQ ID NO: 75 | Tet-kivD-leuDH construct |
| SEQ ID NO: 76 | Tet-kivD-adh2 construct |
| SEQ ID NO: 78 | Tet-leuDH-kivD-adh2 construct |
| SEQ ID NO: 79 | Tet-ilvE-kivD-adh2 construct: |
| SEQ ID NO: 3 | Tet-bkd construct sequence |
| SEQ ID NO: 4 | Tet-leuDH-bkd construct |
| SEQ ID NO: 5 | Tet-livKHMGF construct |
| SEQ ID NO: 6 | pKIKO-lacZ |
| SEQ ID NO: 7 | pTet-livKHMGF sequence |
| SEQ ID NO: 8 | E. coli Nissle 1917 leucine exporter gene leuE |
| SEQ ID NO: 9 | leuE deletion construct |
| SEQ ID NO: 10 | Tet-livKHMGF fragment |
| SEQ ID NO: 11 | Ptac-livJ construct |
| SEQ ID NO: 12 | livJ sequence |
| SEQ ID NO: 13 | - Prp promoter (prpR sequence - underlined; Ribosome binding site - |
| | lower case; start codon of gene of interest (italicized atg) |
| SEQ ID NO: 19 | LeuDH Amino acid sequence Pseudomonas aeruginosa PA01 |
| SEQ ID NO: 20 | leuDH codon-optimized nucleotide sequence Pseudomonas aeruginosa |
| | PA01 |
| SEQ ID NO: 21 | IlvE Amino acid sequence |
| SEQ ID NO: 22 | ilvE nucleotide sequence (E. coli Nissle) |
| SEQ ID NO: 23 | L-AAD Amino acid sequence (Proteus vulgaris) |
| SEQ ID NO: 24 | L-AAD Codon-optimized nucleotide sequence (Proteus vulgaris) |
| SEQ ID NO: 25 | L-AAD Amino acid sequence (Proteus mirabilis) |
| SEQ ID NO: 26 | L-AAD Nucleotide sequence (Proteus mirabilis) |
| SEQ ID NO: 27 | KivD Amino acid sequence (Lactococcus lactis) |
| SEQ ID NO: 28 | kivD Nucleotide sequence (Lactococcus lactis) |
| SEQ ID NO: 29 | kivD Codon-optimized sequence (Lactococcus lactis) |
| SEQ ID NO: 30 | KdcA Amino acid sequence (Lactococcus lactis) |
| SEQ ID NO: 31 | kdcA Nucleotide sequence (Lactococcus lactis) |
| SEQ ID NO: 32 | kdcA Codon-optimized kdcA sequence |
| SEQ ID NO: 33 | THI3/KID1 Amino acid sequence (Saccharomyces cerevisiae) |
| SEQ ID NO: 34 | THI3/KID1 Nucleotide sequence (Saccharomyces cerevisiae) |
| SEQ ID NO: 35 | ARO10 Amino acid sequence (Saccharomyces cerevisiae) |
| SEQ ID NO: 36 | ARO10 Nucleotide sequence (Saccharomyces cerevisiae) |
| SEQ ID NO: 37 | Adh2 Amino acid sequence (Saccharomyces cerevisiae) |
| SEQ ID NO: 38 | adh2 Nucleotide sequence (Saccharomyces cerevisiae) |
| SEQ ID NO: 39 | adh2 Codon-optimized sequence (Saccharomyces cerevisiae) |
| SEQ ID NO: 40 | Adh6 Amino acid sequence (Saccharomyces cerevisiae) |
| SEQ ID NO: 41 | adh6 Codon-optimized sequence (Saccharomyces cerevisiae) |
| SEQ ID NO: 42 | Adh1 Amino acid sequence (Saccharomyces cerevisiae) |
| SEQ ID NO: 43 | adh1 Nucleotide sequence (Saccharomyces cerevisiae) |
| SEQ ID NO: 44 | Adh3 Amino acid sequence (Saccharomyces cerevisiae) |
| SEQ ID NO: 45 | adh3 Nucleotide sequence (Saccharomyces cerevisiae) |
| SEQ ID NO: 46 | Adh4 Amino acid sequence (Saccharomyces cerevisiae) |
| SEQ ID NO: 47 | adh4 Nucleotide sequence (Saccharomyces cerevisiae) |
| SEQ ID NO: 48 | Adh5 Amino acid sequence (Saccharomyces cerevisiae) |
| SEQ ID NO: 49 | adh5 Nucleotide sequence (Saccharomyces cerevisiae) |
| SEQ ID NO: 50 | Adh7 Amino acid sequence (Saccharomyces cerevisiae) |
| SEQ ID NO: 51 | adh7 Nucleotide sequence (Saccharomyces cerevisiae) |
| SEQ ID NO: 52 | SFA1 Amino acid sequence (Saccharomyces cerevisiae) |
| SEQ ID NO: 53 | sfa1 Nucleotide sequence (Saccharomyces cerevisiae) |
| SEQ ID NO: 54 | IlvC amino acid sequence from E. coli Nissle |
| SEQ ID NO: 55 | ilvC gene from E. coli Nissle nucleotide sequence |
| SEQ ID NO: 56 | L-amino acid deaminase L-AAD Codon-optimized sequence |
| | (from Proteus vulgaris) |
| SEQ ID NO: 57 | L-amino acid deaminase L-AAD amino acid sequence |
| | (from Proteus vulgaris) |
| SEQ ID NO: 58 | Leucine dehydrogenase leuDH from Bacillus cereus, Codon-optimized |
| | sequence |
| SEQ ID NO: 59 | Leucine dehydrogenase leuDH from Bacillus cereus, amino acid sequence |
| SEQ ID NO: 60 | Alcohol dehydrogenase YqhD from E. coli, Nucleotide sequence |
| SEQ ID NO: 61 | Alcohol dehydrogenase YqhD from E. coli, amino acid sequence |
| SEQ ID NO: 62 | Aldehyde dehydrogenase PadA from E. coli, Nucleotide sequence |
| SEQ ID NO: 63 | Aldehyde dehydrogenase PadA from E. coli, amino acid sequence |
| SEQ ID NO: 64 | BCAA transporter BrnQ from E. coli, Nucleotide sequence |
| SEQ ID NO: 65 | BCAA transporter BrnQ from E. coli, AA sequence |
| SEQ ID NO: 66 | Isovaleryl-CoA synthetase LbuL from Streptomyces lividans |
| SEQ ID NO: 67 | Isovaleryl-CoA synthetase LbuL from Streptomyces lividans |
| SEQ ID NO: 68 | LiuABCDE operon from Pseudomonas aeruginosa, liuA AA sequences |
| SEQ ID NO: 69 | LiuABCDE operon from Pseudomonas aeruginosa, LiuB AA sequences |
| SEQ ID NO: 70 | LiuABCDE operon from Pseudomonas aeruginosa, LiuC AA sequences |
| SEQ ID NO: 71 | LiuABCDE operon from Pseudomonas aeruginosa, LiuD AA sequences |
| SEQ ID NO: 72 | LiuABCDE operon from Pseudomonas aeruginosa, LiuE AA sequences |
| SEQ ID NO: 73 | LiuABCDE codon optimized sequence |
| SEQ ID NO: 74 | LiuABCDE operon from Pseudomonas aeruginosa, AA sequences |
| SEQ ID NO: 75 | Tet-kivD-leuDH construct |
| SEQ ID NO: 76 | Tet-kivD-adh2 construct |
| SEQ ID NO: 78 | Tet-leuDH-kivD-adh2 construct |
| SEQ ID NO: 79 | Tet-ilvE-kivD-adh2 construct: |
| SEQ ID NO: 91 | Nucleotide sequence of the livKHMGF operon |
| SEQ ID NO: 92 | LivK amino acid sequence |
| SEQ ID NO: 93 | LivK nucleotide sequence |
| SEQ ID NO: 94 | LivH amino acid sequence |
| SEQ ID NO: 95 | LivH nucleotide sequence |
| SEQ ID NO: 96 | LivM amino acid sequence |
| SEQ ID NO: 97 | LivM nucleotide sequence |
| SEQ ID NO: 98 | LivG amino acid sequence |
| SEQ ID NO: 99 | LivG nucleotide sequence |
| SEQ ID NO: 100 | LivF amino acid sequence |
| SEQ ID NO: 101 | LivF nucleotide sequence |
| SEQ ID NO: 14 | FNR responsive regulatory sequence |
| SEQ ID NO: 15 | FNR responsive regulatory sequence |
| SEQ ID NO: 16 | FNR responsive regulatory sequence |
| SEQ ID NO: 17 | FNR responsive regulatory sequence |
| SEQ ID NO: 18 | FNR responsive regulatory sequence |
| SEQ ID NO: 80 | SEQ ID NO; FNR responsive regulatory sequence |
| SEQ ID NO: 81 | SEQ ID NO; FNR responsive regulatory sequence |
| SEQ ID NO: 82 | nirB1 |
| SEQ ID NO: 83 | nirB2 |
| SEQ ID NO: 84 | nirB3 |
| SEQ ID NO: 85 | ydfZ |
| SEQ ID NO: 86 | nirB + RBS |
| SEQ ID NO: 87 | ydfZ + RBS |
| SEQ ID NO: 88 | fnrS1 |
| SEQ ID NO: 89 | fnrS2 |
| SEQ ID NO: 90 | nirB + crp |
| SEQ ID NO: 91 | fnrS + crp |
| SEQ ID NO: 46 | katG |
| SEQ ID NO: 47 | dps |
| SEQ ID NO: 48 | ahpC |
| SEQ ID NO: 49 | oxyS |
| SEQ ID NO: 1 | kivD gene from Lactococcus lactis IFPL730 |
| SEQ ID NO: 2 | Tet-kivD construct |
| SEQ ID NO: 75 | Tet-kivD-leuDH construct |
| SEQ ID NO: 76 | Tet-kivD-adh2 construct |
| SEQ ID NO: 78 | Tet-leuDH-kivD-adh2 construct |
| SEQ ID NO: 79 | Tet-ilvE-kivD-adh2 construct: |
| SEQ ID NO: 3 | Tet-bkd construct sequence |
| SEQ ID NO: 4 | Tet-leuDH-bkd construct |
| SEQ ID NO: 5 | Tet-livKHMGF construct |
| SEQ ID NO: 6 | pKIKO-lacZ |
| SEQ ID NO: 7 | pTet-livKHMGF sequence |
| SEQ ID NO: 8 | E. coli Nissle 1917 leucine exporter gene leuE |
| SEQ ID NO: 9 | leuE deletion construct |
| SEQ ID NO: 10 | Tet-livKHMGF fragment |
| SEQ ID NO: 11 | Ptac-livJ construct |
| SEQ ID NO: 12 | livJ sequence |
| SEQ ID NO: 13 | - Prp promoter (prpR sequence - underlined; Ribosome binding site - |
| | lower case; start codon of gene of interest (italicized atg) |
| SEQ ID NO: 19 | LeuDH Amino acid sequence Pseudomonas aeruginosa PA01 |
| SEQ ID NO: 20 | leuDH codon-optimized nucleotide sequence Pseudomonas aeruginosa |
| | PA01 |
| SEQ ID NO: 21 | IlvE Amino acid sequence |
| SEQ ID NO: 22 | ilvE nucleotide sequence (E. coli Nissle) |
| SEQ ID NO: 23 | L-AAD Amino acid sequence (Proteus vulgaris) |
| SEQ ID NO: 24 | L-AAD Codon-optimized nucleotide sequence (Proteus vulgaris) |
| SEQ ID NO: 25 | L-AAD Amino acid sequence (Proteus mirabilis) |
| SEQ ID NO: 26 | L-AAD Nucleotide sequence (Proteus mirabilis) |
| SEQ ID NO: 27 | KivD Amino acid sequence (Lactococcus lactis) |
| SEQ ID NO: 28 | kivD Nucleotide sequence (Lactococcus lactis) |
| SEQ ID NO: 29 | kivD Codon-optimized sequence (Lactococcus lactis) |
| SEQ ID NO: 30 | KdcA Amino acid sequence (Lactococcus lactis) |
| SEQ ID NO: 31 | kdcA Nucleotide sequence (Lactococcus lactis) |
| SEQ ID NO: 32 | kdcA Codon-optimized kdcA sequence |
| SEQ ID NO: 33 | THI3/KID1 Amino acid sequence (Saccharomyces cerevisiae) |
| SEQ ID NO: 34 | THI3/KID1 Nucleotide sequence (Saccharomyces cerevisiae) |
| SEQ ID NO: 35 | ARO10 Amino acid sequence (Saccharomyces cerevisiae) |
| SEQ ID NO: 36 | ARO10 Nucleotide sequence (Saccharomyces cerevisiae) |
| SEQ ID NO: 37 | Adh2 Amino acid sequence (Saccharomyces cerevisiae) |
| SEQ ID NO: 38 | adh2 Nucleotide sequence (Saccharomyces cerevisiae) |
| SEQ ID NO: 39 | adh2 Codon-optimized sequence (Saccharomyces cerevisiae) |
| SEQ ID NO: 40 | Adh6 Amino acid sequence (Saccharomyces cerevisiae) |
| SEQ ID NO: 41 | adh6 Codon-optimized sequence (Saccharomyces cerevisiae) |
| SEQ ID NO: 42 | Adh1 Amino acid sequence (Saccharomyces cerevisiae) |
| SEQ ID NO: 43 | adh1 Nucleotide sequence (Saccharomyces cerevisiae) |
| SEQ ID NO: 44 | Adh3 Amino acid sequence (Saccharomyces cerevisiae) |
| SEQ ID NO: 45 | adh3 Nucleotide sequence (Saccharomyces cerevisiae) |
| SEQ ID NO: 46 | Adh4 Amino acid sequence (Saccharomyces cerevisiae) |
| SEQ ID NO: 47 | adh4 Nucleotide sequence (Saccharomyces cerevisiae) |
| SEQ ID NO: 48 | Adh5 Amino acid sequence (Saccharomyces cerevisiae) |
| SEQ ID NO: 49 | adh5 Nucleotide sequence (Saccharomyces cerevisiae) |
| SEQ ID NO: 50 | Adh7 Amino acid sequence (Saccharomyces cerevisiae) |
| SEQ ID NO: 51 | adh7 Nucleotide sequence (Saccharomyces cerevisiae) |
| SEQ ID NO: 52 | SFA1 Amino acid sequence (Saccharomyces cerevisiae) |
| SEQ ID NO: 53 | sfa1 Nucleotide sequence (Saccharomyces cerevisiae) |
| SEQ ID NO: 54 | IlvC amino acid sequence from E. coli Nissle |
| SEQ ID NO: 55 | ilvC gene from E. coli Nissle nucleotide sequence |
| SEQ ID NO: 56 | L-amino acid deaminase L-AAD Codon-optimized sequence |
| | (from Proteus vulgaris) |
| SEQ ID NO: 57 | L-amino acid deaminase L-AAD amino acid sequence |
| | (from Proteus vulgaris) |
| SEQ ID NO: 58 | Leucine dehydrogenase leuDH from Bacillus cereus, Codon-optimized |
| | sequence |
| SEQ ID NO: 59 | Leucine dehydrogenase leuDH from Bacillus cereus, amino acid sequence |
| SEQ ID NO: 60 | Alcohol dehydrogenase YqhD from E. coli, Nucleotide sequence |
| SEQ ID NO: 61 | Alcohol dehydrogenase YqhD from E. coli, amino acid sequence |
| SEQ ID NO: 62 | Aldehyde dehydrogenase PadA from E. coli, Nucleotide sequence |
| SEQ ID NO: 63 | Aldehyde dehydrogenase PadA from E. coli, amino acid sequence |
| SEQ ID NO: 64 | BCAA transporter BrnQ from E. coli, Nucleotide sequence |
| SEQ ID NO: 65 | BCAA transporter BrnQ from E. coli, AA sequence |
| SEQ ID NO: 66 | Isovaleryl-CoA synthetase LbuL from Streptomyces lividans |
| SEQ ID NO: 67 | Isovaleryl-CoA synthetase LbuL from Streptomyces lividans |
| SEQ ID NO: 68 | LiuABCDE operon from Pseudomonas aeruginosa, liuA AA sequences |
| SEQ ID NO: 69 | LiuABCDE operon from Pseudomonas aeruginosa, LiuB AA sequences |
| SEQ ID NO: 70 | LiuABCDE operon from Pseudomonas aeruginosa, LiuC AA sequences |
| SEQ ID NO: 71 | LiuABCDE operon from Pseudomonas aeruginosa, LiuD AA sequences |
| SEQ ID NO: 72 | LiuABCDE operon from Pseudomonas aeruginosa, LiuE AA sequences |
| SEQ ID NO: 73 | LiuABCDE codon optimized sequence |
| SEQ ID NO: 74 | LiuABCDE operon from Pseudomonas aeruginosa, AA sequences |
| SEQ ID NO: 75 | Tet-kivD-leuDH construct |
| SEQ ID NO: 76 | Tet-kivD-adh2 construct |
| SEQ ID NO: 78 | Tet-leuDH-kivD-adh2 construct |
| SEQ ID NO: 79 | Tet-ilvE-kivD-adh2 construct: |
| SEQ ID NO: 91 | Nucleotide sequence of the livKHMGF operon |
| SEQ ID NO: 92 | LivK amino acid sequence |
| SEQ ID NO: 93 | LivK nucleotide sequence |
| SEQ ID NO: 94 | LivH amino acid sequence |
| SEQ ID NO: 95 | LivH nucleotide sequence |
| SEQ ID NO: 96 | LivM amino acid sequence |
| SEQ ID NO: 97 | LivM nucleotide sequence |
| SEQ ID NO: 98 | LivG amino acid sequence |
| SEQ ID NO: 99 | LivG nucleotide sequence |
| SEQ ID NO: 100 | LivF amino acid sequence |
| SEQ ID NO: 101 | LivF nucleotide sequence |
| SEQ ID NO: 103 | Arabinose Promoter region |
| SEQ ID NO: 104 | AraC (reverse orientation) |
| SEQ ID NO: 105 | AraC polypeptide |
| SEQ ID NO: 106 | Region comprising rhamnose inducible promoter |
| SEQ ID NO: 107 | Lac Promoter region |
| SEQ ID NO: 108 | LacO |
| SEQ ID NO: 109 | LacI (in reverse orientation) |
| SEQ ID NO: 110 | LacI polypeptide sequence |
| SEQ ID NO: 111 | TetR-tet promoter construct |
| SEQ ID NO: 112 | Region comprising Temperature sensitive promoter |
| SEQ ID NO: 113 | mutant cI857 repressor |
| SEQ ID NO: 114 | RBS and leader region |
| SEQ ID NO: 115 | mutant cI857 repressor polypeptide sequence |
| SEQ ID NO: 116 | PssB promoter |
| SEQ ID NO: 117 | FNR promoter with RBS and leader region (underlined), FNR binding site |
| | bold |
| SEQ ID NO: 118 | FNR binding site |
| SEQ ID NO: 119 | FNR promoter without RBS and leader region |
| SEQ ID NO: 120 | RBS and leader region |
| SEQ ID NO: 121 | LeuDH-kivD-adh2-brnQ construct |
| SEQ ID NO: 122 | Pfnrs-LeuDH-kivD-adh2-brnQ construct (with terminator) (RBS are |
| | underlined) |
| SEQ ID NO: 123 | Tet-LeuDH-kivD-adh2-brnQ construct (tet Repressor is in reverse |
| | orientation and underlined; tet promoter with RBS and leader region is in |
| | bold italics) |
| SEQ ID NO: 124 | Tet-LeuDH-kivD-padA-brnQ construct (tet Repressor is in reverse |
| | orientation and underlined; tet promoter with RBS and leader region is in |
| | bold italics) |
| SEQ ID NO: 125 | LeuDH-kivD-padA-brnQ (RBS are underlined) |
| SEQ ID NO: 126 | Fnrs-LeuDH-kivD-padA-brnQ (RBS are underlined); FNR promoter with |
| | RBS and leader region (underlined), FNR binding site bold |
| SEQ ID NO: 127 | Ptet-LeuDH-kivD-yqhD-brnQ construct tet Repressor is in reverse |
| | orientation and underlined; tet promoter with RBS and leader region is in |
| | bold italics) |
| SEQ ID NO: 128 | LeuDH-kivD-yqhD-brnQ construct (RBS are underlined) |
| SEQ ID NO: 129 | Pfnrs-LeuDH-kivD-yqhD-brnQ construct (RBS are underlined); FNR |
| | promoter with RBS and leader region (underlined), FNR binding site bold |
| SEQ ID NO: 130 | SR36 Primer |
| SEQ ID NO: 131 | SR38 Primer |
| SEQ ID NO: 132 | SR33 Primer |
| SEQ ID NO: 133 | SR34 Primer |
| SEQ ID NO: 134 | SR43 Primer |
| SEQ ID NO: 135 | SR44 Primer |
| SEQ ID NO: 136 | Nucleotide sequences of Pfnr1-lacZ construct, low-copy |
| SEQ ID NO: 137 | Nucleotide sequences of Pfnr2-lacZ construct, low-copy |
| SEQ ID NO: 138 | Nucleotide sequences of Pfnr3-lacZ construct, low-copy |
| SEQ ID NO: 139 | Nucleotide sequences of Pfnr4-lacZ construct, low-copy |
| SEQ ID NO: 140 | Nucleotide sequences of Pfnrs-lacZ construct, low-copy |

Sequences

Gene coding regions are shown in uppercase
SEQ ID NO: 1: kivD gene from Lactococcas lactis IFPL730
ATGTATACAGTAGGAGATTACCTATTAGACCGATTACACGAGTTAGGAATTGAA
GAAATTTTTGGAGTCCCTGGAGACTATAACTTACAATTTTTAGATCAAATTATTTC
CCACAAGGATATGAAATGGGTCGGAAATGCTAATGAATTAAATGCTTCATATAT
GGCTGATGGCTATGCTCGTACTAAAAAAGCTGCCGCATTTCTTACAACCTTTGGA
GTAGGTGAATTGAGTGCAGTTAATGGATTAGCAGGAAGTTACGCCGAAAATTTA
CCAGTAGTAGAAATAGTGGGATCACCTACATCAAAAGTTCAAAATGAAGGAAAA
TTTGTTCATCATACGCTGGCTGACGGTGATTTTAAACACTTTATGAAAATGCACG
AACCTGTTACAGCAGCTCGAACTTTACTGACAGCAGAAAATGCAACCGTTGAAA
TTGACCGAGTACTTTCTGCACTATTAAAAGAAAGAAAACCTGTCTATATCAACTT
ACCAGTTGATGTTGCTGCTGCAAAAGCAGAGAAACCCTCACTCCCTTTGAAAAAG
GAAAACTCAACTTCAAATACAAGTGACCAAGAAATTTTGAACAAAATTCAAGAA
AGCTTGAAAAATGCCAAAAAACCAATCGTGATTACAGGACATGAAATAATTAGT
TTTGGCTTAGAAAAAACAGTCACTCAATTTATTTCAAAGACAAAACTACCTATTA
CGACATTAAACTTTGGTAAAAGTTCAGTTGATGAAGCCCTCCCTTCATTTTTAGG
AATCTATAATGGTACACTCTCAGAGCCTAATCTTAAAGAATTCGTGGAATCAGCC
GACTTCATCTTGATGCTTGGAGTTAAACTCACAGACTCTTCAACAGGAGCCTTCA
CTCATCATTTAAATGAAAATAAAATGATTTCACTGAATATAGATGAAGGAAAAA
TATTTAACGAAAGAATCCAAAATTTTGATTTTGAATCCCTCATCTCCTCTCTCTTA
GACCTAAGCGAAATAGAATACAAAGGAAAATATATCGATAAAAAGCAAGAAGA
CTTTGTTCCATCAAATGCGCTTTTATCACAAGACCGCCTATGGCAAGCAGTTGAA
AACCTAACTCAAAGCAATGAAACAATCGTTGCTGAACAAGGGACATCATTCTTTG
GCGCTTCATCAATTTTCTTAAAATCAAAGAGTCATTTTATTGGTCAACCCTTATGG
GGATCAATTGGATATACATTCCCAGCAGCATTAGGAAGCCAAATTGCAGATAAA
GAAAGCAGACACCTTTTATTTATTGGTGATGGTTCACTTCAACTTACAGTGCAAG
AATTAGGATTAGCAATCAGAGAAAAAATTAATCCAATTTGCTTTATTATCAATAA
TGATGGTTATACAGTCGAAAGAGAAATTCATGGACCAAATCAAAGCTACAATGA
TATTCCAATGTGGAATTACTCAAAATTACCAGAATCGTTTGGAGCAACAGAAGAT
CGAGTAGTCTCAAAAATCGTTAGAACTGAAAATGAATTTGTGTCTGTCATGAAAG
AAGCTCAAGCAGATCCAAATAGAATGTACTGGATTGAGTTAATTTTGGCAAAAG
AAGGTGCACCAAAAGTACTGAAAAAAATGGGCAAACTATTTGCTGAACAAAATA
AATCATAA
SEQ ID NO: 2 Tet-kivD construct
gaattcgTTAAGACCCACTTTCACATTTAAGTTGTTTTTCTAATCCGCATATGATCAAT
TCAAGGCCGAATAAGAAGGCTGGCTCTGCACCTTGGTGATCAAATAATTCGATA
GCTTGTCGTAATAATGGCGGCATACTATCAGTAGTAGGTGTTTCCCTTTCTTCTTT
AGCGACTTGATGCTCTTGATCTTCCAATACGCAACCTAAAGTAAAATGCCCCACA
GCGCTGAGTGCATATAATGCATTCTCTAGTGAAAAACCTTGTTGGCATAAAAAGG
CTAATTGATTTTCGAGAGTTTCATACTGTTTTTCTGTAGGCCGTGTACCTAAATGT
ACTTTTGCTCCATCGCGATGACTTAGTAAAGCACATCTAAAACTTTTAGCGTTATT
ACGTAAAAAATCTTGCCAGCTTTCCCCTTCTAAAGGGCAAAAGTGAGTATGGTGC
CTATCTAACATCTCAATGGCTAAGGCGTCGAGCAAAGCCCGCTTATTTTTTACAT
GCCAATACAATGTAGGCTGCTCTACACCTAGCTTCTGGGCGAGTTTACGGGTTGT
TAAACCTTCGATTCCGACCTCATTAAGCAGCTCTAATGCGCTGTTAATCACTTTAC
TTTTATCTAATCTAGACATCATTAATTcctaatattgagacactctatcattgatagagttatataccactcccta
tcagtgatagagaaaagtgaactctagaaataattagataactttaagaaggagatatacatATGTATACAGTAGGAGA
TTACCTATTAGACCGATTACACGAGTTAGGAATTGAAGAAATTTTTGGAGTCCCT
GGAGACTATAACTTACAATTTTTAGATCAAATTATTTCCCACAAGGATATGAAAT
GGGTCGGAAATGCTAATGAATTAAATGCTTCATATATGGCTGATGGCTATGCTCG
TACTAAAAAAGCTGCCGCATTTCTTACAACCTTTGGAGTAGGTGAATTGAGTGCA
GTTAATGGATTAGCAGGAAGTTACGCCGAAAATTTACCAGTAGTAGAAATAGTG
GGATCACCTACATCAAAAGTTCAAAATGAAGGAAAATTTGTTCATCATACGCTGG
CTGACGGTGATTTTAAACACTTTATGAAAATGCACGAACCTGTTACAGCAGCTCG
AACTTTACTGACAGCAGAAAATGCAACCGTTGAAATTGACCGAGTACTTTCTGCA
CTATTAAAAGAAAGAAAACCTGTCTATATCAACTTACCAGTTGATGTTGCTGCTG
CAAAAGCAGAGAAACCCTCACTCCCTTTGAAAAAGGAAAACTCAACTTCAAATA
CAAGTGACCAAGAAATTTTGAACAAAATTCAAGAAAGCTTGAAAAATGCCAAAA
AACCAATCGTGATTACAGGACATGAAATAATTAGTTTTGGCTTAGAAAAAACAG
TCACTCAATTTATTTCAAAGACAAAACTACCTATTACGACATTAAACTTTGGTAA
AAGTTCAGTTGATGAAGCCCTCCCTTCATTTTTAGGAATCTATAATGGTACACTCT
CAGAGCCTAATCTTAAAGAATTCGTGGAATCAGCCGACTTCATCTTGATGCTTGG
AGTTAAACTCACAGACTCTTCAACAGGAGCCTTCACTCATCATTTAAATGAAAAT
AAAATGATTTCACTGAATATAGATGAAGGAAAAATATTTAACGAAAGAATCCAA
AATTTTGATTTTGAATCCCTCATCTCCTCTCTCTTAGACCTAAGCGAAATAGAATA
CAAAGGAAAATATATCGATAAAAAGCAAGAAGACTTTGTTCCATCAAATGCGCT
TTTATCACAAGACCGCCTATGGCAAGCAGTTGAAAACCTAACTCAAAGCAATGA
AACAATCGTTGCTGAACAAGGGACATCATTCTTTGGCGCTTCATCAATTTTCTTA
AAATCAAAGAGTCATTTTATTGGTCAACCCTTATGGGGATCAATTGGATATACAT
TCCCAGCAGCATTAGGAAGCCAAATTGCAGATAAAGAAAGCAGACACCTTTTAT
TTATTGGTGATGGTTCACTTCAACTTACAGTGCAAGAATTAGGATTAGCAATCAG
AGAAAAAATTAATCCAATTTGCTTTATTATCAATAATGATGGTTATACAGTCGAA
AGAGAAATTCATGGACCAAATCAAAGCTACAATGATATTCCAATGTGGAATTAC
TCAAAATTACCAGAATCGTTTGGAGCAACAGAAGATCGAGTAGTCTCAAAAATC
GTTAGAACTGAAAATGAATTTGTGTCTGTCATGAAAGAAGCTCAAGCAGATCCA
AATAGAATGTACTGGATTGAGTTAATTTTGGCAAAAGAAGGTGCACCAAAAGTA
CTGAAAAAAATGGGCAAACTATTTGCTGAACAAAATAAATCATAAtacgcatggcatgga
tgaattgtataaataa
SEQ ID NO: 75 Tet-kivD-leuDH construct:
gaattcgTTAAGACCCACTTTCACATTTAAGTTGTTTTTCTAATCCGCATATGATCAAT
TCAAGGCCGAATAAGAAGGCTGGCTCTGCACCTTGGTGATCAAATAATTCGATA
GCTTGTCGTAATAATGGCGGCATACTATCAGTAGTAGGTGTTTCCCTTTCTTCTTT
AGCGACTTGATGCTCTTGATCTTCCAATACGCAACCTAAAGTAAAATGCCCCACA
GCGCTGAGTGCATATAATGCATTCTCTAGTGAAAAACCTTGTTGGCATAAAAAGG
CTAATTGATTTTCGAGAGTTTCATACTGTTTTTCTGTAGGCCGTGTACCTAAATGT
ACTTTTGCTCCATCGCGATGACTTAGTAAAGCACATCTAAAACTTTTAGCGTTATT
ACGTAAAAAATCTTGCCAGCTTTCCCCTTCTAAAGGGCAAAAGTGAGTATGGTGC
CTATCTAACATCTCAATGGCTAAGGCGTCGAGCAAAGCCCGCTTATTTTTTACAT
GCCAATACAATGTAGGCTGCTCTACACCTAGCTTCTGGGCGAGTTTACGGGTTGT
TAAACCTTCGATTCCGACCTCATTAAGCAGCTCTAATGCGCTGTTAATCACTTTAC
TTTTATCTAATCTAGACATcattaattcctaatattgagacactctatcattgatagagttatataccactccctatcagtg
atagagaaaagtgaactctagaaataattagataactttaagaaggagatatacatATGTATACAGTAGGAGATTA
CCTATTAGACCGATTACACGAGTTAGGAATTGAAGAAATTTTTGGAGTCCCTGGA
GACTATAACTTACAATTTTTAGATCAAATTATTTCCCACAAGGATATGAAATGGG
TCGGAAATGCTAATGAATTAAATGCTTCATATATGGCTGATGGCTATGCTCGTAC
TAAAAAAGCTGCCGCATTTCTTACAACCTTTGGAGTAGGTGAATTGAGTGCAGTT
AATGGATTAGCAGGAAGTTACGCCGAAAATTTACCAGTAGTAGAAATAGTGGGA
TCACCTACATCAAAAGTTCAAAATGAAGGAAAATTTGTTCATCATACGCTGGCTG
ACGGTGATTTTAAACACTTTATGAAAATGCACGAACCTGTTACAGCAGCTCGAAC
TTTACTGACAGCAGAAAATGCAACCGTTGAAATTGACCGAGTACTTTCTGCACTA
TTAAAAGAAAGAAAACCTGTCTATATCAACTTACCAGTTGATGTTGCTGCTGCAA
AAGCAGAGAAACCCTCACTCCCTTTGAAAAAGGAAAACTCAACTTCAAATACAA
GTGACCAAGAAATTTTGAACAAAATTCAAGAAAGCTTGAAAAATGCCAAAAAAC
CAATCGTGATTACAGGACATGAAATAATTAGTTTTGGCTTAGAAAAAACAGTCAC
TCAATTTATTTCAAAGACAAAACTACCTATTACGACATTAAACTTTGGTAAAAGT
TCAGTTGATGAAGCCCTCCCTTCATTTTTAGGAATCTATAATGGTACACTCTCAGA
GCCTAATCTTAAAGAATTCGTGGAATCAGCCGACTTCATCTTGATGCTTGGAGTT
AAACTCACAGACTCTTCAACAGGAGCCTTCACTCATCATTTAAATGAAAATAAAA
TGATTTCACTGAATATAGATGAAGGAAAAATATTTAACGAAAGAATCCAAAATT
TTGATTTTGAATCCCTCATCTCCTCTCTCTTAGACCTAAGCGAAATAGAATACAA
AGGAAAATATATCGATAAAAAGCAAGAAGACTTTGTTCCATCAAATGCGCTTTTA
TCACAAGACCGCCTATGGCAAGCAGTTGAAAACCTAACTCAAAGCAATGAAACA
ATCGTTGCTGAACAAGGGACATCATTCTTTGGCGCTTCATCAATTTTCTTAAAATC
AAAGAGTCATTTTATTGGTCAACCCTTATGGGGATCAATTGGATATACATTCCCA
GCAGCATTAGGAAGCCAAATTGCAGATAAAGAAAGCAGACACCTTTTATTTATT
GGTGATGGTTCACTTCAACTTACAGTGCAAGAATTAGGATTAGCAATCAGAGAA
AAAATTAATCCAATTTGCTTTATTATCAATAATGATGGTTATACAGTCGAAAGAG
AAATTCATGGACCAAATCAAAGCTACAATGATATTCCAATGTGGAATTACTCAAA
ATTACCAGAATCGTTTGGAGCAACAGAAGATCGAGTAGTCTCAAAAATCGTTAG
AACTGAAAATGAATTTGTGTCTGTCATGAAAGAAGCTCAAGCAGATCCAAATAG
AATGTACTGGATTGAGTTAATTTTGGCAAAAGAAGGTGCACCAAAAGTACTGAA
AAAAATGGGCAAACTATTTGCTGAACAAAATAAATCATAAgaaggagatatacatATGTT
CGACATGATGGATGCAGCCCGCCTGGAAGGCCTGCACCTCGCCCAGGATCCAGC
GACGGGCCTGAAAGCGATCATCGCGATCCATTCCACTCGCCTCGGCCCGGCCTTA
GGCGGCTGTCGTTACCTCCCATATCCGAATGATGAAGCGGCCATCGGCGATGCCA
TTCGCCTGGCGCAGGGCATGTCCTACAAAGCTGCACTTGCGGGTCTGGAACAAG
GTGGTGGCAAGGCGGTGATCATTCGCCCACCCCACTTGGATAATCGCGGTGCCTT
GTTTGAAGCGTTTGGACGCTTTATTGAAAGCCTGGGTGGCCGTTATATCACCGCC
GTTGACTCAGGAACAAGTAGTGCCGATATGGATTGCATCGCCCAACAGACGCGC
CATGTGACTTCAACGACACAAGCCGGCGACCCATCTCCACATACGGCTCTGGGC
GTCTTTGCCGGCATCCGCGCCTCCGCGCAGGCTCGCCTGGGGTCCGATGACCTGG
AAGGCCTGCGTGTCGCGGTTCAGGGCCTTGGCCACGTAGGTTATGCGTTAGCGGA
GCAGCTGGCGGCGGTCGGCGCAGAACTGCTGGTGTGCGACCTGGACCCCGGCCG
CGTCCAGTTAGCGGTGGAGCAACTGGGGGCGCACCCACTGGCCCCTGAAGCATT
GCTCTCTACTCCGTGCGACATTTTAGCGCCTTGTGGCCTGGGCGGCGTGCTCACC
AGCCAGTCGGTGTCACAGTTGCGCTGCGCGGCCGTTGCAGGCGCAGCGAACAAT
CAACTGGAGCGCCCGGAAGTTGCAGACGAACTGGAGGCGCGCGGGATTTTATAT
GCGCCCGATTACGTGATTAACTCGGGAGGACTGATTTATGTGGCGCTGAAGCATC
GCGGTGCTGATCCGCATAGCATTACCGCCCACCTCGCTCGCATCCCTGCACGCCT
GACGGAAATCTATGCGCATGCGCAGGCGGATCATCAGTCGCCTGCGCGCATCGC
CGATCGTCTGGCGGAGCGCATTCTGTACGGCCCGCAGTGA
SEQ ID NO: 76 Tet-kivD-adh2 construct:
gaattcgTTAAGACCCACTTTCACATTTAAGTTGTTTTTCTAATCCGCATATGATCAAT
TCAAGGCCGAATAAGAAGGCTGGCTCTGCACCTTGGTGATCAAATAATTCGATA
GCTTGTCGTAATAATGGCGGCATACTATCAGTAGTAGGTGTTTCCCTTTCTTCTTT
AGCGACTTGATGCTCTTGATCTTCCAATACGCAACCTAAAGTAAAATGCCCCACA
GCGCTGAGTGCATATAATGCATTCTCTAGTGAAAAACCTTGTTGGCATAAAAAGG
CTAATTGATTTTCGAGAGTTTCATACTGTTTTTCTGTAGGCCGTGTACCTAAATGT
ACTTTTGCTCCATCGCGATGACTTAGTAAAGCACATCTAAAACTTTTAGCGTTATT
ACGTAAAAAATCTTGCCAGCTTTCCCCTTCTAAAGGGCAAAAGTGAGTATGGTGC
CTATCTAACATCTCAATGGCTAAGGCGTCGAGCAAAGCCCGCTTATTTTTTACAT
GCCAATACAATGTAGGCTGCTCTACACCTAGCTTCTGGGCGAGTTTACGGGTTGT
TAAACCTTCGATTCCGACCTCATTAAGCAGCTCTAATGCGCTGTTAATCACTTTAC
TTTTATCTAATCTAGACATcattaattcctaatattgagacactctatcattgatagagttatataccactccctatcagtg
atagagaaaagtgaactctagaaataattagataactttaagaaggagatatacatATGTATACAGTAGGAGATTA
CCTATTAGACCGATTACACGAGTTAGGAATTGAAGAAATTTTTGGAGTCCCTGGA
GACTATAACTTACAATTTTTAGATCAAATTATTTCCCACAAGGATATGAAATGGG
TCGGAAATGCTAATGAATTAAATGCTTCATATATGGCTGATGGCTATGCTCGTAC
TAAAAAAGCTGCCGCATTTCTTACAACCTTTGGAGTAGGTGAATTGAGTGCAGTT
AATGGATTAGCAGGAAGTTACGCCGAAAATTTACCAGTAGTAGAAATAGTGGGA
TCACCTACATCAAAAGTTCAAAATGAAGGAAAATTTGTTCATCATACGCTGGCTG
ACGGTGATTTTAAACACTTTATGAAAATGCACGAACCTGTTACAGCAGCTCGAAC
TTTACTGACAGCAGAAAATGCAACCGTTGAAATTGACCGAGTACTTTCTGCACTA
TTAAAAGAAAGAAAACCTGTCTATATCAACTTACCAGTTGATGTTGCTGCTGCAA
AAGCAGAGAAACCCTCACTCCCTTTGAAAAAGGAAAACTCAACTTCAAATACAA
GTGACCAAGAAATTTTGAACAAAATTCAAGAAAGCTTGAAAAATGCCAAAAAAC
CAATCGTGATTACAGGACATGAAATAATTAGTTTTGGCTTAGAAAAAACAGTCAC
TCAATTTATTTCAAAGACAAAACTACCTATTACGACATTAAACTTTGGTAAAAGT
TCAGTTGATGAAGCCCTCCCTTCATTTTTAGGAATCTATAATGGTACACTCTCAGA
GCCTAATCTTAAAGAATTCGTGGAATCAGCCGACTTCATCTTGATGCTTGGAGTT
AAACTCACAGACTCTTCAACAGGAGCCTTCACTCATCATTTAAATGAAAATAAAA
TGATTTCACTGAATATAGATGAAGGAAAAATATTTAACGAAAGAATCCAAAATT
TTGATTTTGAATCCCTCATCTCCTCTCTCTTAGACCTAAGCGAAATAGAATACAA
AGGAAAATATATCGATAAAAAGCAAGAAGACTTTGTTCCATCAAATGCGCTTTTA
TCACAAGACCGCCTATGGCAAGCAGTTGAAAACCTAACTCAAAGCAATGAAACA
ATCGTTGCTGAACAAGGGACATCATTCTTTGGCGCTTCATCAATTTTCTTAAAATC
AAAGAGTCATTTTATTGGTCAACCCTTATGGGGATCAATTGGATATACATTCCCA
GCAGCATTAGGAAGCCAAATTGCAGATAAAGAAAGCAGACACCTTTTATTTATT
GGTGATGGTTCACTTCAACTTACAGTGCAAGAATTAGGATTAGCAATCAGAGAA
AAAATTAATCCAATTTGCTTTATTATCAATAATGATGGTTATACAGTCGAAAGAG
AAATTCATGGACCAAATCAAAGCTACAATGATATTCCAATGTGGAATTACTCAAA
ATTACCAGAATCGTTTGGAGCAACAGAAGATCGAGTAGTCTCAAAAATCGTTAG
AACTGAAAATGAATTTGTGTCTGTCATGAAAGAAGCTCAAGCAGATCCAAATAG
AATGTACTGGATTGAGTTAATTTTGGCAAAAGAAGGTGCACCAAAAGTACTGAA
AAAAATGGGCAAACTATTTGCTGAACAAAATAAATCATAAtaagaaggagatatacatATG
TCTATTCCAGAAACTCAAAAAGCCATTATCTTCTACGAATCCAACGGCAAGTTGG
AGCATAAGGATATCCCAGTTCCAAAGCCAAAGCCCAACGAATTGTTAATCAACG
TCAAGTACTCTGGTGTCTGCCACACCGATTTGCACGCTTGGCATGGTGACTGGCC
ATTGCCAACTAAGTTACCATTAGTTGGTGGTCACGAAGGTGCCGGTGTCGTTGTC
GGCATGGGTGAAAACGTTAAGGGCTGGAAGATCGGTGACTACGCCGGTATCAAA
TGGTTGAACGGTTCTTGTATGGCCTGTGAATACTGTGAATTGGGTAACGAATCCA
ACTGTCCTCACGCTGACTTGTCTGGTTACACCCACGACGGTTCTTTCCAAGAATA
CGCTACCGCTGACGCTGTTCAAGCCGCTCACATTCCTCAAGGTACTGACTTGGCT
GAAGTCGCGCCAATCTTGTGTGCTGGTATCACCGTATACAAGGCTTTGAAGTCTG
CCAACTTGAGAGCAGGCCACTGGGCGGCCATTTCTGGTGCTGCTGGTGGTCTAGG
TTCTTTGGCTGTTCAATATGCTAAGGCGATGGGTTACAGAGTCTTAGGTATTGAT
GGTGGTCCAGGAAAGGAAGAATTGTTTACCTCGCTCGGTGGTGAAGTATTCATCG
ACTTCACCAAAGAGAAGGACATTGTTAGCGCAGTCGTTAAGGCTACCAACGGCG
GTGCCCACGGTATCATCAATGTTTCCGTTTCCGAAGCCGCTATCGAAGCTTCTAC
CAGATACTGTAGGGCGAACGGTACTGTTGTCTTGGTTGGTTTGCCAGCCGGTGCA
AAGTGCTCCTCTGATGTCTTCAACCACGTTGTCAAGTCTATCTCCATTGTCGGCTC
TTACGTGGGGAACAGAGCTGATACCAGAGAAGCCTTAGATTTCTTTGCCAGAGGT
CTAGTCAAGTCTCCAATAAAGGTAGTTGGCTTATCCAGTTTACCAGAAATTTACG
AAAAGATGGAGAAGGGCCAAATTGCTGGTAGATACGTTGTTGACACTTCTAAAT
AA
SEQ ID NO: 78 Tet-leuDH-kivD-adh2 construct
gaattcgTTAAGACCCACTTTCACATTTAAGTTGTTTTTCTAATCCGCATATGATCAAT
TCAAGGCCGAATAAGAAGGCTGGCTCTGCACCTTGGTGATCAAATAATTCGATA
GCTTGTCGTAATAATGGCGGCATACTATCAGTAGTAGGTGTTTCCCTTTCTTCTTT
AGCGACTTGATGCTCTTGATCTTCCAATACGCAACCTAAAGTAAAATGCCCCACA
GCGCTGAGTGCATATAATGCATTCTCTAGTGAAAAACCTTGTTGGCATAAAAAGG
CTAATTGATTTTCGAGAGTTTCATACTGTTTTTCTGTAGGCCGTGTACCTAAATGT
ACTTTTGCTCCATCGCGATGACTTAGTAAAGCACATCTAAAACTTTTAGCGTTATT
ACGTAAAAAATCTTGCCAGCTTTCCCCTTCTAAAGGGCAAAAGTGAGTATGGTGC
CTATCTAACATCTCAATGGCTAAGGCGTCGAGCAAAGCCCGCTTATTTTTTACAT
GCCAATACAATGTAGGCTGCTCTACACCTAGCTTCTGGGCGAGTTTACGGGTTGT
TAAACCTTCGATTCCGACCTCATTAAGCAGCTCTAATGCGCTGTTAATCACTTTAC
TTTTATCTAATCTAGACATcattaattcctaatattgagacactctatcattgatagagttatataccactccctatcagtg
atagagaaaagtgaactctagaaataattagataactttaagaaggagatatacatATGTTCGACATGATGGATGC
AGCCCGCCTGGAAGGCCTGCACCTCGCCCAGGATCCAGCGACGGGCCTGAAAGC
GATCATCGCGATCCATTCCACTCGCCTCGGCCCGGCCTTAGGCGGCTGTCGTTAC
CTCCCATATCCGAATGATGAAGCGGCCATCGGCGATGCCATTCGCCTGGCGCAG
GGCATGTCCTACAAAGCTGCACTTGCGGGTCTGGAACAAGGTGGTGGCAAGGCG
GTGATCATTCGCCCACCCCACTTGGATAATCGCGGTGCCTTGTTTGAAGCGTTTG
GACGCTTTATTGAAAGCCTGGGTGGCCGTTATATCACCGCCGTTGACTCAGGAAC
AAGTAGTGCCGATATGGATTGCATCGCCCAACAGACGCGCCATGTGACTTCAAC
GACACAAGCCGGCGACCCATCTCCACATACGGCTCTGGGCGTCTTTGCCGGCATC
CGCGCCTCCGCGCAGGCTCGCCTGGGGTCCGATGACCTGGAAGGCCTGCGTGTC
GCGGTTCAGGGCCTTGGCCACGTAGGTTATGCGTTAGCGGAGCAGCTGGCGGCG
GTCGGCGCAGAACTGCTGGTGTGCGACCTGGACCCCGGCCGCGTCCAGTTAGCG
GTGGAGCAACTGGGGGCGCACCCACTGGCCCCTGAAGCATTGCTCTCTACTCCGT
GCGACATTTTAGCGCCTTGTGGCCTGGGCGGCGTGCTCACCAGCCAGTCGGTGTC
ACAGTTGCGCTGCGCGGCCGTTGCAGGCGCAGCGAACAATCAACTGGAGCGCCC
GGAAGTTGCAGACGAACTGGAGGCGCGCGGGATTTTATATGCGCCCGATTACGT
GATTAACTCGGGAGGACTGATTTATGTGGCGCTGAAGCATCGCGGTGCTGATCCG
CATAGCATTACCGCCCACCTCGCTCGCATCCCTGCACGCCTGACGGAAATCTATG
CGCATGCGCAGGCGGATCATCAGTCGCCTGCGCGCATCGCCGATCGTCTGGCGG
AGCGCATTCTGTACGGCCCGCAGTGAtaagaaggagatatacatATGTATACAGTAGGAGA
TTACCTATTAGACCGATTACACGAGTTAGGAATTGAAGAAATTTTTGGAGTCCCT
GGAGACTATAACTTACAATTTTTAGATCAAATTATTTCCCACAAGGATATGAAAT
GGGTCGGAAATGCTAATGAATTAAATGCTTCATATATGGCTGATGGCTATGCTCG
TACTAAAAAAGCTGCCGCATTTCTTACAACCTTTGGAGTAGGTGAATTGAGTGCA
GTTAATGGATTAGCAGGAAGTTACGCCGAAAATTTACCAGTAGTAGAAATAGTG
GGATCACCTACATCAAAAGTTCAAAATGAAGGAAAATTTGTTCATCATACGCTGG
CTGACGGTGATTTTAAACACTTTATGAAAATGCACGAACCTGTTACAGCAGCTCG
AACTTTACTGACAGCAGAAAATGCAACCGTTGAAATTGACCGAGTACTTTCTGCA
CTATTAAAAGAAAGAAAACCTGTCTATATCAACTTACCAGTTGATGTTGCTGCTG
CAAAAGCAGAGAAACCCTCACTCCCTTTGAAAAAGGAAAACTCAACTTCAAATA
CAAGTGACCAAGAAATTTTGAACAAAATTCAAGAAAGCTTGAAAAATGCCAAAA
AACCAATCGTGATTACAGGACATGAAATAATTAGTTTTGGCTTAGAAAAAACAG
TCACTCAATTTATTTCAAAGACAAAACTACCTATTACGACATTAAACTTTGGTAA
AAGTTCAGTTGATGAAGCCCTCCCTTCATTTTTAGGAATCTATAATGGTACACTCT
CAGAGCCTAATCTTAAAGAATTCGTGGAATCAGCCGACTTCATCTTGATGCTTGG
AGTTAAACTCACAGACTCTTCAACAGGAGCCTTCACTCATCATTTAAATGAAAAT
AAAATGATTTCACTGAATATAGATGAAGGAAAAATATTTAACGAAAGAATCCAA
AATTTTGATTTTGAATCCCTCATCTCCTCTCTCTTAGACCTAAGCGAAATAGAATA
CAAAGGAAAATATATCGATAAAAAGCAAGAAGACTTTGTTCCATCAAATGCGCT
TTTATCACAAGACCGCCTATGGCAAGCAGTTGAAAACCTAACTCAAAGCAATGA
AACAATCGTTGCTGAACAAGGGACATCATTCTTTGGCGCTTCATCAATTTTCTTA
AAATCAAAGAGTCATTTTATTGGTCAACCCTTATGGGGATCAATTGGATATACAT
TCCCAGCAGCATTAGGAAGCCAAATTGCAGATAAAGAAAGCAGACACCTTTTAT
TTATTGGTGATGGTTCACTTCAACTTACAGTGCAAGAATTAGGATTAGCAATCAG
AGAAAAAATTAATCCAATTTGCTTTATTATCAATAATGATGGTTATACAGTCGAA
AGAGAAATTCATGGACCAAATCAAAGCTACAATGATATTCCAATGTGGAATTAC
TCAAAATTACCAGAATCGTTTGGAGCAACAGAAGATCGAGTAGTCTCAAAAATC
GTTAGAACTGAAAATGAATTTGTGTCTGTCATGAAAGAAGCTCAAGCAGATCCA
AATAGAATGTACTGGATTGAGTTAATTTTGGCAAAAGAAGGTGCACCAAAAGTA
CTGAAAAAAATGGGCAAACTATTTGCTGAACAAAATAAATCATAAtaagaaggagatata
catATGTCTATTCCAGAAACTCAAAAAGCCATTATCTTCTACGAATCCAACGGCAA
GTTGGAGCATAAGGATATCCCAGTTCCAAAGCCAAAGCCCAACGAATTGTTAAT
CAACGTCAAGTACTCTGGTGTCTGCCACACCGATTTGCACGCTTGGCATGGTGAC
TGGCCATTGCCAACTAAGTTACCATTAGTTGGTGGTCACGAAGGTGCCGGTGTCG
TTGTCGGCATGGGTGAAAACGTTAAGGGCTGGAAGATCGGTGACTACGCCGGTA
TCAAATGGTTGAACGGTTCTTGTATGGCCTGTGAATACTGTGAATTGGGTAACGA
ATCCAACTGTCCTCACGCTGACTTGTCTGGTTACACCCACGACGGTTCTTTCCAAG
AATACGCTACCGCTGACGCTGTTCAAGCCGCTCACATTCCTCAAGGTACTGACTT
GGCTGAAGTCGCGCCAATCTTGTGTGCTGGTATCACCGTATACAAGGCTTTGAAG
TCTGCCAACTTGAGAGCAGGCCACTGGGCGGCCATTTCTGGTGCTGCTGGTGGTC
TAGGTTCTTTGGCTGTTCAATATGCTAAGGCGATGGGTTACAGAGTCTTAGGTAT
TGATGGTGGTCCAGGAAAGGAAGAATTGTTTACCTCGCTCGGTGGTGAAGTATTC
ATCGACTTCACCAAAGAGAAGGACATTGTTAGCGCAGTCGTTAAGGCTACCAAC
GGCGGTGCCCACGGTATCATCAATGTTTCCGTTTCCGAAGCCGCTATCGAAGCTT
CTACCAGATACTGTAGGGCGAACGGTACTGTTGTCTTGGTTGGTTTGCCAGCCGG
TGCAAAGTGCTCCTCTGATGTCTTCAACCACGTTGTCAAGTCTATCTCCATTGTCG
GCTCTTACGTGGGGAACAGAGCTGATACCAGAGAAGCCTTAGATTTCTTTGCCAG
AGGTCTAGTCAAGTCTCCAATAAAGGTAGTTGGCTTATCCAGTTTACCAGAAATT
TACGAAAAGATGGAGAAGGGCCAAATTGCTGGTAGATACGTTGTTGACACTTCT
AAATAAtacgcatggcatggatgaa
SEQ ID NO: 79 Tet-ilvE-kivD-adh2 construct:
gaattcgTTAAGACCCACTTTCACATTTAAGTTGTTTTTCTAATCCGCATATGATCAAT
TCAAGGCCGAATAAGAAGGCTGGCTCTGCACCTTGGTGATCAAATAATTCGATA
GCTTGTCGTAATAATGGCGGCATACTATCAGTAGTAGGTGTTTCCCTTTCTTCTTT
AGCGACTTGATGCTCTTGATCTTCCAATACGCAACCTAAAGTAAAATGCCCCACA
GCGCTGAGTGCATATAATGCATTCTCTAGTGAAAAACCTTGTTGGCATAAAAAGG
CTAATTGATTTTCGAGAGTTTCATACTGTTTTTCTGTAGGCCGTGTACCTAAATGT
ACTTTTGCTCCATCGCGATGACTTAGTAAAGCACATCTAAAACTTTTAGCGTTATT
ACGTAAAAAATCTTGCCAGCTTTCCCCTTCTAAAGGGCAAAAGTGAGTATGGTGC
CTATCTAACATCTCAATGGCTAAGGCGTCGAGCAAAGCCCGCTTATTTTTTACAT
GCCAATACAATGTAGGCTGCTCTACACCTAGCTTCTGGGCGAGTTTACGGGTTGT
TAAACCTTCGATTCCGACCTCATTAAGCAGCTCTAATGCGCTGTTAATCACTTTAC
TTTTATCTAATCTAGACATcattaattcctaatattgagacactctatcattgatagagttatataccactccctatcagtg
atagagaaaagtgaactctagaaataattagataactttaagaaggagatatacatATGACCACGAAGAAAGCTGA
TTACATTTGGTTCAATGGGGAGATGGTTCGCTGGGAAGACGCGAAGGTGCATGT
GATGTCGCACGCGCTGCACTATGGCACCTCGGTTTTTGAAGGCATCCGTTGCTAC
GACTCGCACAAAGGACCGGTTGTATTCCGCCATCGTGAGCATATGCAGCGTCTGC
ATGACTCCGCCAAAATCTATCGCTTCCCGGTTTCGCAGAGCATTGATGAGCTGAT
GGAAGCTTGTCGTGACGTGATCCGCAAAAACAATCTCACCAGCGCCTATATCCGT
CCGCTGATCTTCGTTGGTGATGTTGGCATGGGCGTAAACCCGCCAGCGGGATACT
CAACCGACGTGATTATCGCCGCTTTCCCGTGGGGAGCGTATCTGGGCGCAGAAGC
GCTGGAGCAGGGGATCGATGCGATGGTTTCCTCCTGGAACCGCGCAGCACCAAA
CACCATCCCGACGGCGGCAAAAGCCGGTGGTAACTACCTCTCTTCCCTGCTGGTG
GGTAGCGAAGCGCGCCGCCACGGTTATCAGGAAGGTATCGCGTTGGATGTGAAT
GGTTACATCTCTGAAGGCGCAGGCGAAAACCTGTTTGAAGTGAAAGACGGCGTG
CTGTTCACCCCACCGTTCACCTCATCCGCGCTGCCGGGTATTACCCGTGATGCCA
TCATCAAACTGGCAAAAGAGCTGGGAATTGAAGTGCGTGAGCAGGTGCTGTCGC
GCGAATCCCTGTACCTGGCGGATGAAGTGTTTATGTCCGGTACGGCGGCAGAAAT
CACGCCAGTGCGCAGCGTAGACGGTATTCAGGTTGGCGAAGGCCGTTGTGGCCC
GGTTACCAAACGCATTCAGCAAGCCTTCTTCGGCCTCTTCACTGGCGAAACCGAA
GATAAATGGGGCTGGTTAGATCAAGTTAATCAATAAtaagaaggagatatacatATGTATA
CAGTAGGAGATTACCTATTAGACCGATTACACGAGTTAGGAATTGAAGAAATTTT
TGGAGTCCCTGGAGACTATAACTTACAATTTTTAGATCAAATTATTTCCCACAAG
GATATGAAATGGGTCGGAAATGCTAATGAATTAAATGCTTCATATATGGCTGATG
GCTATGCTCGTACTAAAAAAGCTGCCGCATTTCTTACAACCTTTGGAGTAGGTGA
ATTGAGTGCAGTTAATGGATTAGCAGGAAGTTACGCCGAAAATTTACCAGTAGT
AGAAATAGTGGGATCACCTACATCAAAAGTTCAAAATGAAGGAAAATTTGTTCA
TCATACGCTGGCTGACGGTGATTTTAAACACTTTATGAAAATGCACGAACCTGTT
ACAGCAGCTCGAACTTTACTGACAGCAGAAAATGCAACCGTTGAAATTGACCGA
GTACTTTCTGCACTATTAAAAGAAAGAAAACCTGTCTATATCAACTTACCAGTTG
ATGTTGCTGCTGCAAAAGCAGAGAAACCCTCACTCCCTTTGAAAAAGGAAAACT
CAACTTCAAATACAAGTGACCAAGAAATTTTGAACAAAATTCAAGAAAGCTTGA
AAAATGCCAAAAAACCAATCGTGATTACAGGACATGAAATAATTAGTTTTGGCTT
AGAAAAAACAGTCACTCAATTTATTTCAAAGACAAAACTACCTATTACGACATTA
AACTTTGGTAAAAGTTCAGTTGATGAAGCCCTCCCTTCATTTTTAGGAATCTATA
ATGGTACACTCTCAGAGCCTAATCTTAAAGAATTCGTGGAATCAGCCGACTTCAT
CTTGATGCTTGGAGTTAAACTCACAGACTCTTCAACAGGAGCCTTCACTCATCAT
TTAAATGAAAATAAAATGATTTCACTGAATATAGATGAAGGAAAAATATTTAAC
GAAAGAATCCAAAATTTTGATTTTGAATCCCTCATCTCCTCTCTCTTAGACCTAAG
CGAAATAGAATACAAAGGAAAATATATCGATAAAAAGCAAGAAGACTTTGTTCC
ATCAAATGCGCTTTTATCACAAGACCGCCTATGGCAAGCAGTTGAAAACCTAACT
CAAAGCAATGAAACAATCGTTGCTGAACAAGGGACATCATTCTTTGGCGCTTCAT
CAATTTTCTTAAAATCAAAGAGTCATTTTATTGGTCAACCCTTATGGGGATCAATT
GGATATACATTCCCAGCAGCATTAGGAAGCCAAATTGCAGATAAAGAAAGCAGA
CACCTTTTATTTATTGGTGATGGTTCACTTCAACTTACAGTGCAAGAATTAGGATT
AGCAATCAGAGAAAAAATTAATCCAATTTGCTTTATTATCAATAATGATGGTTAT
ACAGTCGAAAGAGAAATTCATGGACCAAATCAAAGCTACAATGATATTCCAATG
TGGAATTACTCAAAATTACCAGAATCGTTTGGAGCAACAGAAGATCGAGTAGTC
TCAAAAATCGTTAGAACTGAAAATGAATTTGTGTCTGTCATGAAAGAAGCTCAA
GCAGATCCAAATAGAATGTACTGGATTGAGTTAATTTTGGCAAAAGAAGGTGCA
CCAAAAGTACTGAAAAAAATGGGCAAACTATTTGCTGAACAAAATAAATCATAAt
aagaaggagatatacatATGTCTATTCCAGAAACTCAAAAAGCCATTATCTTCTACGAATC
CAACGGCAAGTTGGAGCATAAGGATATCCCAGTTCCAAAGCCAAAGCCCAACGA
ATTGTTAATCAACGTCAAGTACTCTGGTGTCTGCCACACCGATTTGCACGCTTGG
CATGGTGACTGGCCATTGCCAACTAAGTTACCATTAGTTGGTGGTCACGAAGGTG
CCGGTGTCGTTGTCGGCATGGGTGAAAACGTTAAGGGCTGGAAGATCGGTGACT
ACGCCGGTATCAAATGGTTGAACGGTTCTTGTATGGCCTGTGAATACTGTGAATT
GGGTAACGAATCCAACTGTCCTCACGCTGACTTGTCTGGTTACACCCACGACGGT
TCTTTCCAAGAATACGCTACCGCTGACGCTGTTCAAGCCGCTCACATTCCTCAAG
GTACTGACTTGGCTGAAGTCGCGCCAATCTTGTGTGCTGGTATCACCGTATACAA
GGCTTTGAAGTCTGCCAACTTGAGAGCAGGCCACTGGGCGGCCATTTCTGGTGCT
GCTGGTGGTCTAGGTTCTTTGGCTGTTCAATATGCTAAGGCGATGGGTTACAGAG
TCTTAGGTATTGATGGTGGTCCAGGAAAGGAAGAATTGTTTACCTCGCTCGGTGG
TGAAGTATTCATCGACTTCACCAAAGAGAAGGACATTGTTAGCGCAGTCGTTAAG
GCTACCAACGGCGGTGCCCACGGTATCATCAATGTTTCCGTTTCCGAAGCCGCTA
TCGAAGCTTCTACCAGATACTGTAGGGCGAACGGTACTGTTGTCTTGGTTGGTTT
GCCAGCCGGTGCAAAGTGCTCCTCTGATGTCTTCAACCACGTTGTCAAGTCTATC
TCCATTGTCGGCTCTTACGTGGGGAACAGAGCTGATACCAGAGAAGCCTTAGATT
TCTTTGCCAGAGGTCTAGTCAAGTCTCCAATAAAGGTAGTTGGCTTATCCAGTTT
ACCAGAAATTTACGAAAAGATGGAGAAGGGCCAAATTGCTGGTAGATACGTTGT
TGACACTTCTAAATAAtacgcatggcatggatgaa
SEQ ID NO: 3: Tet-bkd construct sequence
gtaaaacgacggccagtgaattcgTTAAGACCCACTTTCACATTTAAGTTGTTTTTCTAATCCGC
ATATGATCAATTCAAGGCCGAATAAGAAGGCTGGCTCTGCACCTTGGTGATCAA
ATAATTCGATAGCTTGTCGTAATAATGGCGGCATACTATCAGTAGTAGGTGTTTC
CCTTTCTTCTTTAGCGACTTGATGCTCTTGATCTTCCAATACGCAACCTAAAGTAA
AATGCCCCACAGCGCTGAGTGCATATAATGCATTCTCTAGTGAAAAACCTTGTTG
GCATAAAAAGGCTAATTGATTTTCGAGAGTTTCATACTGTTTTTCTGTAGGCCGT
GTACCTAAATGTACTTTTGCTCCATCGCGATGACTTAGTAAAGCACATCTAAAAC
TTTTAGCGTTATTACGTAAAAAATCTTGCCAGCTTTCCCCTTCTAAAGGGCAAAA
GTGAGTATGGTGCCTATCTAACATCTCAATGGCTAAGGCGTCGAGCAAAGCCCGC
TTATTTTTTACATGCCAATACAATGTAGGCTGCTCTACACCTAGCTTCTGGGCGAG
TTTACGGGTTGTTAAACCTTCGATTCCGACCTCATTAAGCAGCTCTAATGCGCTGT
TAATCACTTTACTTTTATCTAATCTAGACATcattaattcctaatattgagacactctatcattgatagagtta
attaccactccctatcagtgatagagaaaagtgaactctagaaataattagataactttaagaaggagatatacatATGTCCGAC
TACGAGCCACTCCGCTTGCACGTGCCGGAGCCGACAGGTCGTCCCGGCTGCAAA
ACGGATTTCTCTTACCTGCACTTATCTCCCGCAGGTGAAGTCCGCAAACCGCCTG
TCGACGTGGAGCCTGCAGAAACCAGCGATTTGGCATATTCGCTGGTGCGTGTGCT
CGATGATGATGGACATGCAGTGGGTCCGTGGAATCCGCAGCTCTCAAACGAACA
GCTGCTGCGTGGAATGCGCGCGATGCTGAAGACGCGTCTGTTCGATGCTCGCATG
TTGACTGCGCAGCGCCAAAAAAAATTGAGTTTTTATATGCAGTGCTTAGGAGAAG
AGGCAATCGCGACTGCCCATACACTGGCCCTGCGCGATGGTGATATGTGTTTTCC
GACGTACCGTCAGCAGGGGATTCTTATTACACGTGAGTATCCGCTTGTGGATATG
ATCTGCCAGCTGCTGTCGAATGAAGCGGACCCCCTGAAAGGCCGTCAACTGCCG
ATCATGTACAGCAGTAAGGAGGCTGGCTTCTTTAGCATCTCGGGCAATCTTGCGA
CTCAGTTTATTCAGGCGGTGGGGTGGGGGATGGCAAGCGCAATCAAAGGGGATA
CCCGCATTGCATCCGCATGGATTGGCGATGGCGCTACCGCGGAAAGCGATTTTCA
TACGGCGCTGACCTTTGCTCACGTTTATCGCGCACCGGTGATCCTCAATGTGGTC
AACAACCAGTGGGCGATTTCGACGTTTCAGGCCATCGCGGGCGGCGAGGGCACC
ACGTTCGCGAACCGTGGCGTGGGTTGCGGCATTGCGAGCCTCCGTGTGGACGGG
AACGATTTTTTGGCCGTGTATGCGGCGAGCGAATGGGCGGCAGAACGCGCACGC
CGTAACTTGGGACCGTCCCTGATCGAATGGGTAACTTATCGCGCGGGCCCACACA
GCACGAGCGACGATCCGTCAAAGTATCGCCCTGCGGATGATTGGACCAATTTTCC
GCTGGGTGACCCGATTGCGCGTCTGAAACGTCACATGATCGGTTTGGGTATTTGG
AGCGAAGAACAGCACGAAGCTACGCACAAAGCGCTGGAAGCGGAAGTCCTGGC
GGCGCAGAAGCAGGCCGAAAGCCATGGCACTCTGATTGACGGCCGTGTGCCGTC
TGCAGCCTCTATGTTCGAAGATGTTTATGCCGAGTTACCCGAGCACTTACGTCGC
CAGCGCCAGGAGCTCGGGGTATGAACGCCATGAACCCGCAGCATGAAAACGCGC
AAACCGTGACCTCCATGACGATGATTCAGGCCCTGCGCTCGGCGATGGATATTAT
GTTAGAACGTGACGATGACGTCGTGGTGTTTGGTCAGGACGTAGGGTATTTTGGG
GGAGTGTTTCGTTGTACCGAGGGGTTGCAAAAGAAGTATGGTACGAGTCGCGTCT
TCGATGCACCGATCAGCGAATCAGGCATTATCGGCGCTGCCGTGGGCATGGGTG
CATATGGCTTGCGCCCTGTGGTTGAAATTCAGTTTGCAGATTATGTATATCCCGC
GTCTGACCAACTGATTAGTGAGGCGGCACGCCTCCGCTACCGTAGCGCGGGCGA
TTTCATTGTCCCGATGACCGTCCGCATGCCTTGTGGAGGGGGCATTTACGGTGGC
CAAACGCATTCTCAGAGTCCAGAAGCCATGTTCACACAAGTGTGCGGTCTTCGCA
CCGTGATGCCATCTAATCCTTATGACGCCAAAGGATTACTGATTGCGTGCATCGA
AAACGACGATCCGGTTATCTTTTTAGAACCCAAACGTCTGTACAACGGTCCTTTC
GACGGTCATCACGACCGTCCTGTCACGCCGTGGAGCAAACATCCGGCATCGCAA
GTCCCGGATGGGTATTATAAAGTGCCTCTGGACAAAGCAGCGATTGTCCGCCCTG
GTGCAGCCCTTACAGTCCTGACGTATGGTACCATGGTGTACGTGGCGCAGGCCGC
GGCAGATGAAACCGGCCTCGATGCGGAGATTATCGACCTCCGCAGTCTGTGGCC
GCTGGACTTGGAAACTATCGTCGCGAGTGTGAAAAAGACCGGTCGTTGTGTTATT
GCCCATGAAGCGACTCGTACCTGCGGCTTTGGCGCCGAACTGATGTCCCTGGTGC
AGGAACACTGTTTTCACCATCTTGAGGCTCCGATTGAACGCGTCACTGGCTGGGA
CACACCGTACCCTCATGCGCAGGAATGGGCCTATTTCCCGGGCCCAGCGCGCGTG
GGAGCCGCCTTTAAACGCGTGATGGAGGTCTGAATGGGTACCCACGTTATTAAA
ATGCCTGATATTGGTGAAGGCATCGCGGAGGTAGAGCTGGTTGAATGGCACGTT
CAAGTGGGTGATAGCGTGAATGAAGATCAGGTACTCGCGGAAGTAATGACGGAC
AAAGCAACGGTTGAAATCCCGTCCCCTGTTGCTGGCCGCATCTTGGCACTGGGTG
GCCAGCCGGGACAAGTTATGGCGGTGGGAGGAGAATTAATTCGCCTGGAAGTGG
AGGGTGCCGGAAACCTGGCGGAGTCTCCGGCCGCAGCTACGCCCGCCGCTCCGG
TGGCAGCAACTCCGGAAAAACCTAAAGAAGCACCGGTTGCAGCGCCAAAAGCA
GCTGCCGAAGCACCCCGTGCGCTTCGTGATTCTGAAGCGCCGCGCCAACGCCGCC
AGCCGGGGGAACGCCCATTAGCATCACCGGCCGTCCGTCAGCGTGCCCGCGACC
TGGGAATCGAGCTGCAGTTTGTTCAGGGCTCTGGCCCAGCCGGCCGCGTGCTTCA
TGAGGACCTGGATGCGTATCTTACGCAGGATGGAAGTGTTGCTCGTTCAGGCGGC
GCTGCGCAGGGTTACGCGGAACGCCATGATGAACAGGCAGTCCCGGTGATCGGT
CTGCGCCGCAAAATTGCCCAGAAGATGCAGGATGCTAAACGCCGCATTCCTCAC
TTCAGTTACGTCGAAGAGATTGACGTAACCGATCTGGAAGCCCTGCGCGCTCACT
TGAATCAGAAATGGGGCGGGCAACGTGGTAAACTGACGCTGCTGCCTTTCCTCGT
CCGCGCAATGGTCGTCGCATTACGCGATTTCCCGCAACTGAATGCTCGCTATGAT
GATGAAGCGGAAGTAGTGACGCGTTACGGGGCCGTTCATGTTGGTATCGCGACC
CAGTCAGATAATGGGCTCATGGTTCCGGTGTTGCGCCATGCAGAAAGCCGTGACC
TGTGGGGTAATGCGTCGGAAGTTGCGCGTCTGGCCGAAGCGGCGCGTTCCGGTA
AAGCGCAACGTCAGGAACTGAGCGGCTCCACGATTACCCTGTCAAGCCTTGGTGT
GTTGGGAGGGATTGTATCCACGCCAGTCATTAATCACCCGGAAGTTGCAATCGTT
GGTGTTAACCGTATTGTGGAGCGCCCTATGGTTGTTGGTGGTAATATTGTAGTAC
GTAAAATGATGAATCTGAGCTCTTCGTTTGATCATCGCGTGGTGGACGGCATGGA
TGCTGCGGCTTTTATTCAAGCCGTGCGCGGTTTGTTAGAACATCCTGCCACCCTGT
TCCTGGAGTAAgcgATGAGTCAGATTTTAAAAACCTCGCTCCTGATCGTTGGCGGC
GGGCCAGGCGGCTATGTGGCGGCGATCCGCGCCGGCCAGCTGGGGATTCCAACG
GTGTTGGTTGAGGGCGCCGCTTTGGGCGGTACTTGCCTGAATGTGGGGTGCATTC
CGAGCAAAGCGTTGATCCATGCTGCCGAAGAGTACCTTAAAGCGCGCCACTATG
CATCACGTTCCGCGCTGGGCATCCAGGTGCAAGCACCTTCAATTGACATCGCCCG
CACCGTGGAATGGAAAGACGCCATTGTGGACCGTTTGACTTCGGGTGTGGCGGCT
CTGCTGAAAAAGCATGGTGTGGATGTAGTACAAGGATGGGCACGCATCCTCGAC
GGCAAGAGCGTGGCGGTTGAACTGGCGGGCGGGGGGTCGCAGCGCATCGAGTGT
GAACATCTGCTTCTGGCGGCGGGCTCACAAAGCGTTGAATTACCCATCCTGCCTC
TGGGGGGCAAAGTAATCAGCAGCACCGAAGCATTAGCTCCGGGGTCGTTGCCAA
AACGTCTGGTGGTTGTGGGTGGCGGTTATATTGGTCTGGAGCTGGGCACTGCATA
TCGCAAGCTGGGTGTTGAAGTTGCTGTGGTGGAGGCACAACCCCGCATCCTGCCG
GGCTACGATGAGGAACTGACTAAGCCGGTGGCCCAAGCGCTGCGCCGTCTGGGT
GTAGAACTGTACCTGGGTCATTCATTGCTGGGACCGAGTGAAAACGGCGTTCGCG
TGCGTGATGGGGCTGGCGAAGAACGTGAGATCGCCGCGGACCAGGTCCTTGTCG
CAGTTGGCCGCAAACCGCGTTCAGAGGGTTGGAACCTGGAGTCTCTCGGTTTAGA
CATGAATGGGCGTGCCGTAAAAGTGGACGATCAGTGCCGTACAAGCATGCGTAA
CGTATGGGCCATTGGCGACCTGGCGGGCGAACCGATGCTGGCGCACCGCGCTAT
GGCGCAAGGAGAAATGGTCGCCGAATTGATTGCGGGCAAACGCCGTCAGTTTGC
GCCGGTTGCAATTCCTGCAGTCTGTTTTACGGATCCGGAAGTGGTGGTGGCGGGT
CTGAGTCCGGAACAGGCCAAAGATGCGGGTCTGGATTGCCTGGTCGCGTCATTCC
CGTTCGCAGCCAACGGCCGCGCCATGACGTTGGAAGCTAACGAAGGCTTTGTCC
GCGTGGTGGCACGTCGTGACAACCATCTGGTGGTTGGTTGGCAGGCGGTCGGTA
AAGCTGTGTCTGAATTAAGCACCGCGTTCGCACAATCTCTGGAAATGGGCGCTCG
CCTCGAAGACATTGCAGGCACAATCCACGCGCACCCCACCCTGGGTGAAGCTGT
TCAGGAAGCGGCACTCCGTGCCTTAGGTCACGCCCTGCACATTTGA
SEQ ID NO: 4: Tet-leuDH-bkd construct
gtaaaacgacggccagtgaattcgTTAAGACCCACTTTCACATTTAAGTTGTTTTTCTAATCCGC
ATATGATCAATTCAAGGCCGAATAAGAAGGCTGGCTCTGCACCTTGGTGATCAA
ATAATTCGATAGCTTGTCGTAATAATGGCGGCATACTATCAGTAGTAGGTGTTTC
CCTTTCTTCTTTAGCGACTTGATGCTCTTGATCTTCCAATACGCAACCTAAAGTAA
AATGCCCCACAGCGCTGAGTGCATATAATGCATTCTCTAGTGAAAAACCTTGTTG
GCATAAAAAGGCTAATTGATTTTCGAGAGTTTCATACTGTTTTTCTGTAGGCCGT
GTACCTAAATGTACTTTTGCTCCATCGCGATGACTTAGTAAAGCACATCTAAAAC
TTTTAGCGTTATTACGTAAAAAATCTTGCCAGCTTTCCCCTTCTAAAGGGCAAAA
GTGAGTATGGTGCCTATCTAACATCTCAATGGCTAAGGCGTCGAGCAAAGCCCGC
TTATTTTTTACATGCCAATACAATGTAGGCTGCTCTACACCTAGCTTCTGGGCGAG
TTTACGGGTTGTTAAACCTTCGATTCCGACCTCATTAAGCAGCTCTAATGCGCTGT
TAATCACTTTACTTTTATCTAATCTAGACATcattaattcctaatattgagacactctatcattgatagagtta
attaccactccctatcagtgatagagaaaagtgaactctagaaataattagataactttaagaaggagatatacatATGTTCGAT
ATGATGGATGCAGCCCGCCTGGAAGGCCTGCACCTCGCCCAGGATCCAGCGACG
GGCCTGAAAGCGATCATCGCGATCCATTCCACTCGCCTCGGCCCGGCCTTAGGCG
GCTGTCGTTACCTCCCATATCCGAATGATGAAGCGGCCATCGGCGATGCCATTCG
CCTGGCGCAGGGCATGTCCTACAAAGCTGCACTTGCGGGTCTGGAACAAGGTGG
TGGCAAGGCGGTGATCATTCGCCCACCCCACTTGGATAATCGCGGTGCCTTGTTT
GAAGCGTTTGGACGCTTTATTGAAAGCCTGGGTGGCCGTTATATCACCGCCGTTG
ACTCAGGAACAAGTAGTGCCGATATGGATTGCATCGCCCAACAGACGCGCCATG
TGACTTCAACGACACAAGCCGGCGACCCATCTCCACATACGGCTCTGGGCGTCTT
TGCCGGCATCCGCGCCTCCGCGCAGGCTCGCCTGGGGTCCGATGACCTGGAAGG
CCTGCGTGTCGCGGTTCAGGGCCTTGGCCACGTAGGTTATGCGTTAGCGGAGCAG
CTGGCGGCGGTCGGCGCAGAACTGCTGGTGTGCGACCTGGACCCCGGCCGCGTC
CAGTTAGCGGTGGAGCAACTGGGGGCGCACCCACTGGCCCCTGAAGCATTGCTC
TCTACTCCGTGCGACATTTTAGCGCCTTGTGGCCTGGGCGGCGTGCTCACCAGCC
AGTCGGTGTCACAGTTGCGCTGCGCGGCCGTTGCAGGCGCAGCGAACAATCAAC
TGGAGCGCCCGGAAGTTGCAGACGAACTGGAGGCGCGCGGGATTTTATATGCGC
CCGATTACGTGATTAACTCGGGAGGACTGATTTATGTGGCGCTGAAGCATCGCGG
TGCTGATCCGCATAGCATTACCGCCCACCTCGCTCGCATCCCTGCACGCCTGACG
GAAATCTATGCGCATGCGCAGGCGGATCATCAGTCGCCTGCGCGCATCGCCGAT
CGTCTGGCGGAGCGCATTCTGTACGGCCCGCAATAAtgaaggagatatacatATGTCCGAC
TACGAGCCACTCCGCTTGCACGTGCCGGAGCCGACAGGTCGTCCCGGCTGCAAA
ACGGATTTCTCTTACCTGCACTTATCTCCCGCAGGTGAAGTCCGCAAACCGCCTG
TCGACGTGGAGCCTGCAGAAACCAGCGATTTGGCATATTCGCTGGTGCGTGTGCT
CGATGATGATGGACATGCAGTGGGTCCGTGGAATCCGCAGCTCTCAAACGAACA
GCTGCTGCGTGGAATGCGCGCGATGCTGAAGACGCGTCTGTTCGATGCTCGCATG
TTGACTGCGCAGCGCCAAAAAAAATTGAGTTTTTATATGCAGTGCTTAGGAGAAG
AGGCAATCGCGACTGCCCATACACTGGCCCTGCGCGATGGTGATATGTGTTTTCC
GACGTACCGTCAGCAGGGGATTCTTATTACACGTGAGTATCCGCTTGTGGATATG
ATCTGCCAGCTGCTGTCGAATGAAGCGGACCCCCTGAAAGGCCGTCAACTGCCG
ATCATGTACAGCAGTAAGGAGGCTGGCTTCTTTAGCATCTCGGGCAATCTTGCGA
CTCAGTTTATTCAGGCGGTGGGGTGGGGGATGGCAAGCGCAATCAAAGGGGATA
CCCGCATTGCATCCGCATGGATTGGCGATGGCGCTACCGCGGAAAGCGATTTTCA
TACGGCGCTGACCTTTGCTCACGTTTATCGCGCACCGGTGATCCTCAATGTGGTC
AACAACCAGTGGGCGATTTCGACGTTTCAGGCCATCGCGGGCGGCGAGGGCACC
ACGTTCGCGAACCGTGGCGTGGGTTGCGGCATTGCGAGCCTCCGTGTGGACGGG
AACGATTTTTTGGCCGTGTATGCGGCGAGCGAATGGGCGGCAGAACGCGCACGC
CGTAACTTGGGACCGTCCCTGATCGAATGGGTAACTTATCGCGCGGGCCCACACA
GCACGAGCGACGATCCGTCAAAGTATCGCCCTGCGGATGATTGGACCAATTTTCC
GCTGGGTGACCCGATTGCGCGTCTGAAACGTCACATGATCGGTTTGGGTATTTGG
AGCGAAGAACAGCACGAAGCTACGCACAAAGCGCTGGAAGCGGAAGTCCTGGC
GGCGCAGAAGCAGGCCGAAAGCCATGGCACTCTGATTGACGGCCGTGTGCCGTC
TGCAGCCTCTATGTTCGAAGATGTTTATGCCGAGTTACCCGAGCACTTACGTCGC
CAGCGCCAGGAGCTCGGGGTATGAACGCCATGAACCCGCAGCATGAAAACGCGC
AAACCGTGACCTCCATGACGATGATTCAGGCCCTGCGCTCGGCGATGGATATTAT
GTTAGAACGTGACGATGACGTCGTGGTGTTTGGTCAGGACGTAGGGTATTTTGGG
GGAGTGTTTCGTTGTACCGAGGGGTTGCAAAAGAAGTATGGTACGAGTCGCGTCT
TCGATGCACCGATCAGCGAATCAGGCATTATCGGCGCTGCCGTGGGCATGGGTG
CATATGGCTTGCGCCCTGTGGTTGAAATTCAGTTTGCAGATTATGTATATCCCGC
GTCTGACCAACTGATTAGTGAGGCGGCACGCCTCCGCTACCGTAGCGCGGGCGA
TTTCATTGTCCCGATGACCGTCCGCATGCCTTGTGGAGGGGGCATTTACGGTGGC
CAAACGCATTCTCAGAGTCCAGAAGCCATGTTCACACAAGTGTGCGGTCTTCGCA
CCGTGATGCCATCTAATCCTTATGACGCCAAAGGATTACTGATTGCGTGCATCGA
AAACGACGATCCGGTTATCTTTTTAGAACCCAAACGTCTGTACAACGGTCCTTTC
GACGGTCATCACGACCGTCCTGTCACGCCGTGGAGCAAACATCCGGCATCGCAA
GTCCCGGATGGGTATTATAAAGTGCCTCTGGACAAAGCAGCGATTGTCCGCCCTG
GTGCAGCCCTTACAGTCCTGACGTATGGTACCATGGTGTACGTGGCGCAGGCCGC
GGCAGATGAAACCGGCCTCGATGCGGAGATTATCGACCTCCGCAGTCTGTGGCC
GCTGGACTTGGAAACTATCGTCGCGAGTGTGAAAAAGACCGGTCGTTGTGTTATT
GCCCATGAAGCGACTCGTACCTGCGGCTTTGGCGCCGAACTGATGTCCCTGGTGC
AGGAACACTGTTTTCACCATCTTGAGGCTCCGATTGAACGCGTCACTGGCTGGGA
CACACCGTACCCTCATGCGCAGGAATGGGCCTATTTCCCGGGCCCAGCGCGCGTG
GGAGCCGCCTTTAAACGCGTGATGGAGGTCTGAATGGGTACCCACGTTATTAAA
ATGCCTGATATTGGTGAAGGCATCGCGGAGGTAGAGCTGGTTGAATGGCACGTT
CAAGTGGGTGATAGCGTGAATGAAGATCAGGTACTCGCGGAAGTAATGACGGAC
AAAGCAACGGTTGAAATCCCGTCCCCTGTTGCTGGCCGCATCTTGGCACTGGGTG
GCCAGCCGGGACAAGTTATGGCGGTGGGAGGAGAATTAATTCGCCTGGAAGTGG
AGGGTGCCGGAAACCTGGCGGAGTCTCCGGCCGCAGCTACGCCCGCCGCTCCGG
TGGCAGCAACTCCGGAAAAACCTAAAGAAGCACCGGTTGCAGCGCCAAAAGCA
GCTGCCGAAGCACCCCGTGCGCTTCGTGATTCTGAAGCGCCGCGCCAACGCCGCC
AGCCGGGGGAACGCCCATTAGCATCACCGGCCGTCCGTCAGCGTGCCCGCGACC
TGGGAATCGAGCTGCAGTTTGTTCAGGGCTCTGGCCCAGCCGGCCGCGTGCTTCA
TGAGGACCTGGATGCGTATCTTACGCAGGATGGAAGTGTTGCTCGTTCAGGCGGC
GCTGCGCAGGGTTACGCGGAACGCCATGATGAACAGGCAGTCCCGGTGATCGGT
CTGCGCCGCAAAATTGCCCAGAAGATGCAGGATGCTAAACGCCGCATTCCTCAC
TTCAGTTACGTCGAAGAGATTGACGTAACCGATCTGGAAGCCCTGCGCGCTCACT
TGAATCAGAAATGGGGCGGGCAACGTGGTAAACTGACGCTGCTGCCTTTCCTCGT
CCGCGCAATGGTCGTCGCATTACGCGATTTCCCGCAACTGAATGCTCGCTATGAT
GATGAAGCGGAAGTAGTGACGCGTTACGGGGCCGTTCATGTTGGTATCGCGACC
CAGTCAGATAATGGGCTCATGGTTCCGGTGTTGCGCCATGCAGAAAGCCGTGACC
TGTGGGGTAATGCGTCGGAAGTTGCGCGTCTGGCCGAAGCGGCGCGTTCCGGTA
AAGCGCAACGTCAGGAACTGAGCGGCTCCACGATTACCCTGTCAAGCCTTGGTGT
GTTGGGAGGGATTGTATCCACGCCAGTCATTAATCACCCGGAAGTTGCAATCGTT
GGTGTTAACCGTATTGTGGAGCGCCCTATGGTTGTTGGTGGTAATATTGTAGTAC
GTAAAATGATGAATCTGAGCTCTTCGTTTGATCATCGCGTGGTGGACGGCATGGA
TGCTGCGGCTTTTATTCAAGCCGTGCGCGGTTTGTTAGAACATCCTGCCACCCTGT
TCCTGGAGTAAgcgATGAGTCAGATTTTAAAAACCTCGCTCCTGATCGTTGGCGGC
GGGCCAGGCGGCTATGTGGCGGCGATCCGCGCCGGCCAGCTGGGGATTCCAACG
GTGTTGGTTGAGGGCGCCGCTTTGGGCGGTACTTGCCTGAATGTGGGGTGCATTC
CGAGCAAAGCGTTGATCCATGCTGCCGAAGAGTACCTTAAAGCGCGCCACTATG
CATCACGTTCCGCGCTGGGCATCCAGGTGCAAGCACCTTCAATTGACATCGCCCG
CACCGTGGAATGGAAAGACGCCATTGTGGACCGTTTGACTTCGGGTGTGGCGGCT
CTGCTGAAAAAGCATGGTGTGGATGTAGTACAAGGATGGGCACGCATCCTCGAC
GGCAAGAGCGTGGCGGTTGAACTGGCGGGCGGGGGGTCGCAGCGCATCGAGTGT
GAACATCTGCTTCTGGCGGCGGGCTCACAAAGCGTTGAATTACCCATCCTGCCTC
TGGGGGGCAAAGTAATCAGCAGCACCGAAGCATTAGCTCCGGGGTCGTTGCCAA
AACGTCTGGTGGTTGTGGGTGGCGGTTATATTGGTCTGGAGCTGGGCACTGCATA
TCGCAAGCTGGGTGTTGAAGTTGCTGTGGTGGAGGCACAACCCCGCATCCTGCCG
GGCTACGATGAGGAACTGACTAAGCCGGTGGCCCAAGCGCTGCGCCGTCTGGGT
GTAGAACTGTACCTGGGTCATTCATTGCTGGGACCGAGTGAAAACGGCGTTCGCG
TGCGTGATGGGGCTGGCGAAGAACGTGAGATCGCCGCGGACCAGGTCCTTGTCG
CAGTTGGCCGCAAACCGCGTTCAGAGGGTTGGAACCTGGAGTCTCTCGGTTTAGA
CATGAATGGGCGTGCCGTAAAAGTGGACGATCAGTGCCGTACAAGCATGCGTAA
CGTATGGGCCATTGGCGACCTGGCGGGCGAACCGATGCTGGCGCACCGCGCTAT
GGCGCAAGGAGAAATGGTCGCCGAATTGATTGCGGGCAAACGCCGTCAGTTTGC
GCCGGTTGCAATTCCTGCAGTCTGTTTTACGGATCCGGAAGTGGTGGTGGCGGGT
CTGAGTCCGGAACAGGCCAAAGATGCGGGTCTGGATTGCCTGGTCGCGTCATTCC
CGTTCGCAGCCAACGGCCGCGCCATGACGTTGGAAGCTAACGAAGGCTTTGTCC
GCGTGGTGGCACGTCGTGACAACCATCTGGTGGTTGGTTGGCAGGCGGTCGGTA
AAGCTGTGTCTGAATTAAGCACCGCGTTCGCACAATCTCTGGAAATGGGCGCTCG
CCTCGAAGACATTGCAGGCACAATCCACGCGCACCCCACCCTGGGTGAAGCTGT
TCAGGAAGCGGCACTCCGTGCCTTAGGTCACGCCCTGCACATTTGA
SEQ ID NO: 5: Tet-livKHMGF construct
ccagtgaattcgTTAAGACCCACTTTCACATTTAAGTTGTTTTTCTAATCCGCATATGATC
AATTCAAGGCCGAATAAGAAGGCTGGCTCTGCACCTTGGTGATCAAATAATTCG
ATAGCTTGTCGTAATAATGGCGGCATACTATCAGTAGTAGGTGTTTCCCTTTCTTC
TTTAGCGACTTGATGCTCTTGATCTTCCAATACGCAACCTAAAGTAAAATGCCCC
ACAGCGCTGAGTGCATATAATGCATTCTCTAGTGAAAAACCTTGTTGGCATAAAA
AGGCTAATTGATTTTCGAGAGTTTCATACTGTTTTTCTGTAGGCCGTGTACCTAAA
TGTACTTTTGCTCCATCGCGATGACTTAGTAAAGCACATCTAAAACTTTTAGCGTT
ATTACGTAAAAAATCTTGCCAGCTTTCCCCTTCTAAAGGGCAAAAGTGAGTATGG
TGCCTATCTAACATCTCAATGGCTAAGGCGTCGAGCAAAGCCCGCTTATTTTTTA
CATGCCAATACAATGTAGGCTGCTCTACACCTAGCTTCTGGGCGAGTTTACGGGT
TGTTAAACCTTCGATTCCGACCTCATTAAGCAGCTCTAATGCGCTGTTAATCACTT
TACTTTTATCTAATCTAGACATcattaattcctaatattgagacactctatcattgatagagttatataccactccctat
cagtgatagagaaaagtgaactctagaaataattagataactttaagaaggagatatacatATGAAACGGAATGCGAA
AACTATCATCGCAGGGATGATTGCACTGGCAATTTCACACACCGCTATGGCTGAC
GATATTAAAGTCGCCGTTGTCGGCGCGATGTCCGGCCCGATTGCCCAGTGGGGCG
ATATGGAATTTAACGGCGCGCGTCAGGCAATTAAAGACATTAATGCCAAAGGGG
GAATTAAGGGCGATAAACTGGTTGGCGTGGAATATGACGACGCATGCGACCCGA
AACAAGCCGTTGCGGTCGCCAACAAAATCGTTAATGACGGCATTAAATACGTTAT
TGGTCATCTGTGTTCTTCTTCTACCCAGCCTGCGTCAGATATCTATGAAGACGAA
GGTATTCTGATGATCTCGCCGGGAGCGACCAACCCGGAGCTGACCCAACGCGGT
TATCAACACATTATGCGTACTGCCGGGCTGGACTCTTCCCAGGGGCCAACGGCGG
CAAAATACATTCTTGAGACGGTGAAGCCCCAGCGCATCGCCATCATTCACGACA
AACAACAGTATGGCGAAGGGCTGGCGCGTTCGGTGCAGGACGGGCTGAAAGCGG
CTAACGCCAACGTCGTCTTCTTCGACGGTATTACCGCCGGGGAGAAAGATTTCTC
CGCGCTGATCGCCCGCCTGAAAAAAGAAAACATCGACTTCGTTTACTACGGCGGT
TACTACCCGGAAATGGGGCAGATGCTGCGCCAGGCCCGTTCCGTTGGCCTGAAA
ACCCAGTTTATGGGGCCGGAAGGTGTGGGTAATGCGTCGTTGTCGAACATTGCCG
GTGATGCCGCCGAAGGCATGTTGGTCACTATGCCAAAACGCTATGACCAGGATC
CGGCAAACCAGGGCATCGTTGATGCGCTGAAAGCAGACAAGAAAGATCCGTCCG
GGCCTTATGTCTGGATCACCTACGCGGCGGTGCAATCTCTGGCGACTGCCCTTGA
GCGTACCGGCAGCGATGAGCCGCTGGCGCTGGTGAAAGATTTAAAAGCTAACGG
TGCAAACACCGTGATTGGGCCGCTGAACTGGGATGAAAAAGGCGATCTTAAGGG
ATTTGATTTTGGTGTCTTCCAGTGGCACGCCGACGGTTCATCCACGGCAGCCAAG
TGAtcatcccaccgcccgtaaaatgcgggcgggatagaaaggttaccttATGTCTGAGCAGTTTTTGTATTTC
TTGCAGCAGATGTTTAACGGCGTCACGCTGGGCAGTACCTACGCGCTGATAGCCA
TCGGCTACACCATGGTTTACGGCATTATCGGCATGATCAACTTCGCCCACGGCGA
GGTTTATATGATTGGCAGCTACGTCTCATTTATGATCATCGCCGCGCTGATGATG
ATGGGCATTGATACCGGCTGGCTGCTGGTAGCTGCGGGATTCGTCGGCGCAATCG
TCATTGCCAGCGCCTACGGCTGGAGTATCGAACGGGTGGCTTACCGCCCGGTGCG
TAACTCTAAGCGCCTGATTGCACTCATCTCTGCAATCGGTATGTCCATCTTCCTGC
AAAACTACGTCAGCCTGACCGAAGGTTCGCGCGACGTGGCGCTGCCGAGCCTGT
TTAACGGTCAGTGGGTGGTGGGGCATAGCGAAAACTTCTCTGCCTCTATTACCAC
CATGCAGGCGGTGATCTGGATTGTTACCTTCCTCGCCATGCTGGCGCTGACGATT
TTCATTCGCTATTCCCGCATGGGTCGCGCGTGTCGTGCCTGCGCGGAAGATCTGA
AAATGGCGAGTCTGCTTGGCATTAACACCGACCGGGTGATTGCGCTGACCTTTGT
GATTGGCGCGGCGATGGCGGCGGTGGCGGGTGTGCTGCTCGGTCAGTTCTACGG
CGTCATTAACCCCTACATCGGCTTTATGGCCGGGATGAAAGCCTTTACCGCGGCG
GTGCTCGGTGGGATTGGCAGCATTCCGGGAGCGATGATTGGCGGCCTGATTCTGG
GGATTGCGGAGGCGCTCTCTTCTGCCTATCTGAGTACGGAATATAAAGATGTGGT
gTCATTCGCCCTGCTGATTCTGGTGCTGCTGGTGATGCCGACCGGTATTCTGGGTC
GCCCGGAGGTAGAGAAAGTATGAAACCGATGCATATTGCAATGGCGCTGCTCTC
TGCCGCGATGTTCTTTGTGCTGGCGGGCGTCTTTATGGGCGTGCAACTGGAGCTG
GATGGCACCAAACTGGTGGTCGACACGGCTTCGGATGTCCGTTGGCAGTGGGTGT
TTATCGGCACGGCGGTGGTCTTTTTCTTCCAGCTTTTGCGACCGGCTTTCCAGAAA
GGGTTGAAAAGCGTTTCCGGACCGAAGTTTATTCTGCCCGCCATTGATGGCTCCA
CGGTGAAGCAGAAACTGTTCCTCGTGGCGCTGTTGGTGCTTGCGGTGGCGTGGCC
GTTTATGGTTTCACGCGGGACGGTGGATATTGCCACCCTGACCATGATCTACATT
ATCCTCGGTCTgGGGCTGAACGTGGTTGTTGGTCTTTCTGGTCTGCTGGTGCTGGG
GTACGGCGGTTTTTACGCCATCGGCGCTTACACTTTTGCGCTGCTCAATCACTATT
ACGGCTTGGGCTTCTGGACCTGCCTGCCGATTGCTGGATTAATGGCAGCGGCGGC
GGGCTTCCTGCTCGGTTTTCCGGTGCTGCGTTTGCGCGGTGACTATCTGGCGATCG
TTACCCTCGGTTTCGGCGAAATTGTGCGCATATTGCTGCTCAATAACACCGAAAT
TACCGGCGGCCCGAACGGAATCAGTCAGATCCCGAAACCGACACTCTTCGGACT
CGAGTTCAGCCGTACCGCTCGTGAAGGCGGCTGGGACACGTTCAGTAATTTCTTT
GGCCTGAAATACGATCCCTCCGATCGTGTCATCTTCCTCTACCTGGTGGCGTTGCT
GCTGGTGGTGCTAAGCCTGTTTGTCATTAACCGCCTGCTGCGGATGCCGCTGGGG
CGTGCGTGGGAAGCGTTGCGTGAAGATGAAATCGCCTGCCGTTCGCTGGGCTTAA
GCCCGCGTCGTATCAAGCTGACTGCCTTTACCATAAGTGCCGCGTTTGCCGGTTTT
GCCGGAACGCTGTTTGCGGCGCGTCAGGGCTTTGTCAGCCCGGAATCCTTCACCT
TTGCCGAATCGGCGTTTGTGCTGGCGATAGTGGTGCTCGGCGGTATGGGCTCGCA
ATTTGCGGTGATTCTGGCGGCAATTTTGCTGGTGGTGTCGCGCGAGTTGATGCGT
GATTTCAACGAATACAGCATGTTAATGCTCGGTGGTTTGATGGTGCTGATGATGA
TCTGGCGTCCGCAGGGCTTGCTGCCCATGACGCGCCCGCAACTGAAGCTGAAAA
ACGGCGCAGCGAAAGGAGAGCAGGCATGAGTCAGCCATTATTATCTGTTAACGG
CCTGATGATGCGCTTCGGCGGCCTGCTGGCGGTGAACAACGTCAATCTTGAACTG
TACCCGCAGGAGATCGTCTCGTTAATCGGCCCTAACGGTGCCGGAAAAACCACG
GTTTTTAACTGTCTGACCGGATTCTACAAACCCACCGGCGGCACCATTTTACTGC
GCGATCAGCACCTGGAAGGTTTACCGGGGCAGCAAATTGCCCGCATGGGCGTGG
TGCGCACCTTCCAGCATGTGCGTCTGTTCCGTGAAATGACGGTAATTGAAAACCT
GCTGGTGGCGCAGCATCAGCAACTGAAAACCGGGCTGTTCTCTGGCCTGTTGAAA
ACGCCATCCTTCCGTCGCGCCCAGAGCGAAGCGCTCGACCGCGCCGCGACCTGG
CTTGAGCGCATTGGTTTGCTGGAACACGCCAACCGTCAGGCGAGTAACCTGGCCT
ATGGTGACCAGCGCCGTCTTGAGATTGCCCGCTGCATGGTGACGCAGCCGGAGA
TTTTAATGCTCGACGAACCTGCGGCAGGTCTTAACCCGAAAGAGACGAAAGAGC
TGGATGAGCTGATTGCCGAACTGCGCAATCATCACAACACCACTATCTTGTTGAT
TGAACACGATATGAAGCTGGTGATGGGAATTTCGGACCGAATTTACGTGGTCAAT
CAGGGGACGCCGCTGGCAAACGGTACGCCGGAGCAGATCCGTAATAACCCGGAC
GTGATCCGTGCCTATTTAGGTGAGGCATAAGATGGAAAAAGTCATGTTGTCCTTT
GACAAAGTCAGCGCCCACTACGGCAAAATCCAGGCGCTGCATGAGGTGAGCCTG
CATATCAATCAGGGCGAGATTGTCACGCTGATTGGCGCGAACGGGGCGGGGAAA
ACCACCTTGCTCGGCACGTTATGCGGCGATCCGCGTGCCACCAGCGGGCGAATTG
TGTTTGATGATAAAGACATTACCGACTGGCAGACAGCGAAAATCATGCGCGAAG
CGGTGGCGATTGTCCCGGAAGGGCGTCGCGTCTTCTCGCGGATGACGGTGGAAG
AGAACCTGGCGATGGGCGGTTTTTTTGCTGAACGCGACCAGTTCCAGGAGCGCAT
AAAGTGGGTGTATGAGCTGTTTCCACGTCTGCATGAGCGCCGTATTCAGCGGGCG
GGCACCATGTCCGGCGGTGAACAGCAGATGCTGGCGATTGGTCGTGCGCTGATG
AGCAACCCGCGTTTGCTACTGCTTGATGAGCCATCGCTCGGTCTTGCGCCGATTA
TCATCCAGCAAATTTTCGACACCATCGAGCAGCTGCGCGAGCAGGGGATGACTA
TCTTTCTCGTCGAGCAGAACGCCAACCAGGCGCTAAAGCTGGCGGATCGCGGCT
ACGTGCTGGAAAACGGCCATGTAGTGCTTTCCGATACTGGTGATGCGCTGCTGGC
GAATGAAGCGGTGAGAAGTGCGTATTTAGGCGGGTAA
SEQ ID NO: 6: pKIKO-lacZ
agattgcagcattacacgtcttgagcgattgtgtaggctggagctgcttcgaagacctatactactagagaataggaacttcggaatag
gaacttcatttaaatggcgcgccttacgccccgccctgccacTCATCGCAGTACTGTTGTATTCATTAAGC
ATCTGCCGACATGGAAGCCATCACAAACGGCATGATGAACCTGAATCGCCAGCG
GCATCAGCACCTTGTCGCCTTGCGTATAATATTTGCCCATGGTGAAAACGGGGGC
GAAGAAGTTGTCCATATTGGCCACGTTTAAATCAAAACTGGTGAAACTCACCCAG
GGATTGGCTGAGACGAAAAACATATTCTCAATAAACCCTTTAGGGAAATAGGCC
AGGTTTTCACCGTAACACGCCACATCTTGCGAATATATGTGTAGAAACTGCCGGA
AATCGTCGTGGTATTCACTCCAGAGCGATGAAAACGTTTCAGTTTGCTCATGGAA
AACGGTGTAACAAGGGTGAACACTATCCCATATCACCAGCTCACCGTCTTTCATT
GCCATACGTAATTCCGGATGAGCATTCATCAGGCGGGCAAGAATGTGAATAAAG
GCCGGATAAAACTTGTGCTTATTTTTCTTTACGGTCTTTAAAAAGGCCGTAATATC
CAGCTGAACGGTCTGGTTATAGGTACATTGAGCAACTGACTGAAATGCCTCAAA
ATGTTCTTTACGATGCCATTGGGATATATCAACGGTGGTATATCCAGTGATTTTTT
TCTCCATatagcaccttagctcctgaaaatctcgacaactcaaaaaatacgcccggtagtgatcttatttcattatggtgaaagag
gaacctcttacgtgccgatcaacgtctcattacgccaaaagaggcccagggcacccggtatcaacagggacaccaggatttatttatt
ctgcgaagtgatcaccgtcacaggtaggcgcgccgaagacctatactactagagaataggaacttcggaataggaactaaggagga
tattcatatggaccatggctaattccTTGCCGTTTTCATCATATTTAATCAGCGACTGATCCACCC
AGTCCCAGACGAAGCCGCCCTGTAAACGGGGGTACTGACGAAACGCCTGCCAGT
ATTTAGCGAAGCCGCCAAGACTGTTACCCATCGCGTGGGCATATTCGCAAAGGAT
CAGCGGGCGCATTTCTCCAGGCAGCGAAAGCCATTTTTTGATGGACCATTTCGGC
ACCGCCGGGAAGGGCTGGTCTTCATCCACGCGCGCGTACATCGGGCAAATAATA
TCGGTGGCCGTGGTGTCGGCTCCGCCGCCTTCATACTGTACCGGGCGGGAAGGAT
CGACAGATTTGATCCAGCGATACAGCGCGTCGTGATTAGCGCCGTGGCCTGATTC
ATTCCCCAGCGACCAGATGATCACACTCGGGTGATTACGATCGCGCTGCACCATC
CGCGTTACGCGTTCGCTCATCGCGGGTAGCCAGCGCGGATCATCGGTCAGACGAT
TCATTGGCACCATGCCGTGGGTTTCAATATTGGCTTCATCCACCACATACAGGCC
GTAGCGGTCGCACAGCGTGTACCACAGCGGATGGTTCGGATAATGCGAACAGCG
CACGGCGTTAAAGTTGTTCTGCTTCATCAGCAGGATATCCTGCACCATCGTCTGC
TCATCCATGACCTGACCATGCAGAGGATGATGCTCGTGACGGTTAACGCCGCGA
ATCAGCAACGGCTTGCCGTTCAGCAGCAGCAGACCATTTTCAATCCGCACCTCGC
GGAAACCGACGTCGCAGGCTTCTGCTTCAATCAGCGTGCCGTCGGCGGTGTGCAG
TTCAACCACTGCACGATAGAGATTCGGGATTTCGGCGCTCCACAGTTCCGGATTT
TCAACGTTCAGGCGTAGTGTGACGCGATCGGCATAACCGCCACGCTCATCGATAA
TTTCACCcatgtcagccgttaagtgacctgtgtcactgaaaattgcatgagaggctctaagggcactcagtgcgttacatccctg
gcttgagtccacaaccgttaaaccttaaaagcataaaagccttatatattcattattcttataaaacttaaaaccttagaggctatttaagtt
gctgatttatattaatatattgacaaacatgagagcttagtacgtgaaacatgagagcttagtacgttagccatgagagcttagtacgttag
ccatgagggatagacgttaaacatgagagcttagtacgttaaacatgagagcttagtacgtgaaacatgagagcttagtacgtactatc
aacaggagaactgcggatcagcggccgcaaaaattaaaaatgaagattaaatcaatctaaagtatatatgagtaaacttggtctgaca
gTTACCAATGCTTAATCAGTGAGGCACCTATCTCAGCGATCTGTCTATTTCGTTCA
TCCATAGTTGCCTGACTCCCCGTCGTGTAGATAACTACGATACGGGAGGGCTTAC
CATCTGGCCCCAGTGCTGCAATGATACCGCGAGACCCACGCTCACCGGCTCCAG
ATTTATCAGCAATAAACCAGCCAGCCGGAAGGGCCGAGCGCAGAAGTGGTCCTG
CAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGATCAA
GGCGAGTTACATGATCCCCCATGTTGTGCAAAAAAGCGGTTAGCTCCTTCGGTCC
TCCGATCGTTGTCAGAAGTAAGTTGGCCGCAGTGTTATCACTCATGGTTATGGCA
GCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCT
TGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGT
TGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAA
AAGGGAATAAGGGCGACACGGAAATGTTGAATACTCATactcaccatttcaatattattgaagcat
ttatcagggttattgtctcatgagcggatacatatttgaatgtatttagaaaaataaacaaataggggaccgcgACTGACGGGC
TCCAGGAGTCGTCGCCACCAATCCCCATATGGAAACCGTCGATATTCAGCCATGT
GCCTTCTTCCGCGTGCAGCAGATGGCGATGGCTGGTTTCCATCAGTTGTTGTTGG
CTGTAGCGGCTGATGTTGAACTGGAAGTCGCCGCGCCACTGGTGTGGGCCATAAT
TCAATTCGCGCGTCCCGCAGCGCAGACCGTTTTCGCTCGGGAAGACGTACGGGGT
ATACATGTCTGACAATGGCAGATCCCAGCGGTCAAAACAGGCTGCAGTAAGGCG
GTCGGGATAGTTTTCTTGCGGCCCCAGGCCGAGCCAGTTTACCCGCTCTGAGACC
TGCGCCAGCTGGCAGGTCAGGCCAATCCGCGCCGGATGCGGTGTATCGCTTGCC
ACCGCAACATCCACATTGATGACCATCTCACCGTGCCCATCAATCCGGTAGGTTT
TCCGGCTGATAAATAAGGTTTTCCCCTGATGCTGCCACGCGTGGGCGGTTGTAAT
CAGCACCGCGTCGGCAAGTGTATCTGCCGTGCACTGCAACAACGCCGCTTCGGCC
TGGTAATGGCCCGCCGCCTTCCAGCGTTCGACCCAGGCGTTAGGGTCAATGCGGG
TCGCTTCACTTACGCCAATGTCGTTATCCAGCGGCGCACGGGTGAACTGATCGCG
CAGCGGGGTCAGCAGTTGTTTTTCATCGCCAATCCACATCTGTGAAAGAAAGCCT
GACTGGCGGTTAAATTGCCAACGCTTATTACCCAGCTCGATGCAAAAATCCGTTC
CGCTGGTGGTCAGTTGAGGGATGGCGTGGGACGCGGAGGGGAGTGTCACGCTGA
GGTTTTCCGCCAGACGCCATTGCTGCCAGGCGCTGATGTGTCCGGCTTCTGACCA
TGCGGTCGCGTTTGGTTGCACTACGCGTACCGTTAGCCAGAGTcacataccccgaaaagtgc
cacctgcatcgatggccccccgatggtagtgtggggtctccccatgcgagagtagggaactgccaggcatcaaataaaacgaaagg
ctcagtcgaaagactgggccatcgattatctgagtagtcggtgaacgctctcctgagtaggacaaatccgccgggagcggatttgaa
cgttgcgaagcaacggcccggagggtggcgggcaggacgcccgccataaactgccaggcatcaaattaagcagaaggccatcct
gacggatggccatttgcgtggccagtgccaagcttgcatgc
SEQ ID NO: 7: pTet-livKHMGF sequence
agattgcagcattacacgtcttgagcgattgtgtaggctggagctgcttcgaagacctatactactagagaataggaacttcggaatag
gaacttcatttaaatggcgcgccttacgccccgccctgccacTCATCGCAGTACTGTTGTATTCATTAAGC
ATCTGCCGACATGGAAGCCATCACAAACGGCATGATGAACCTGAATCGCCAGCG
GCATCAGCACCTTGTCGCCTTGCGTATAATATTTGCCCATGGTGAAAACGGGGGC
GAAGAAGTTGTCCATATTGGCCACGTTTAAATCAAAACTGGTGAAACTCACCCAG
GGATTGGCTGAGACGAAAAACATATTCTCAATAAACCCTTTAGGGAAATAGGCC
AGGTTTTCACCGTAACACGCCACATCTTGCGAATATATGTGTAGAAACTGCCGGA
AATCGTCGTGGTATTCACTCCAGAGCGATGAAAACGTTTCAGTTTGCTCATGGAA
AACGGTGTAACAAGGGTGAACACTATCCCATATCACCAGCTCACCGTCTTTCATT
GCCATACGTAATTCCGGATGAGCATTCATCAGGCGGGCAAGAATGTGAATAAAG
GCCGGATAAAACTTGTGCTTATTTTTCTTTACGGTCTTTAAAAAGGCCGTAATATC
CAGCTGAACGGTCTGGTTATAGGTACATTGAGCAACTGACTGAAATGCCTCAAA
ATGTTCTTTACGATGCCATTGGGATATATCAACGGTGGTATATCCAGTGATTTTTT
TCTCCATatagcaccttagctcctgaaaatctcgacaactcaaaaaatacgcccggtagtgatcttatttcattatggtgaaagag
gaacctcttacgtgccgatcaacgtctcattacgccaaaagaggcccagggcttcccggtatcaacagggacaccaggatttatttatt
ctgcgaagtgatcaccgtcacaggtaggcgcgccgaagacctatactactagagaataggaacttcggaataggaactaaggagga
tattcatatggaccatggctaattccTTGCCGTTTTCATCATATTTAATCAGCGACTGATCCACCC
AGTCCCAGACGAAGCCGCCCTGTAAACGGGGGTACTGACGAAACGCCTGCCAGT
ATTTAGCGAAGCCGCCAAGACTGTTACCCATCGCGTGGGCATATTCGCAAAGGAT
CAGCGGGCGCATTTCTCCAGGCAGCGAAAGCCATTTTTTGATGGACCATTTCGGC
ACCGCCGGGAAGGGCTGGTCTTCATCCACGCGCGCGTACATCGGGCAAATAATA
TCGGTGGCCGTGGTGTCGGCTCCGCCGCCTTCATACTGTACCGGGCGGGAAGGAT
CGACAGATTTGATCCAGCGATACAGCGCGTCGTGATTAGCGCCGTGGCCTGATTC
ATTCCCCAGCGACCAGATGATCACACTCGGGTGATTACGATCGCGCTGCACCATC
CGCGTTACGCGTTCGCTCATCGCGGGTAGCCAGCGCGGATCATCGGTCAGACGAT
TCATTGGCACCATGCCGTGGGTTTCAATATTGGCTTCATCCACCACATACAGGCC
GTAGCGGTCGCACAGCGTGTACCACAGCGGATGGTTCGGATAATGCGAACAGCG
CACGGCGTTAAAGTTGTTCTGCTTCATCAGCAGGATATCCTGCACCATCGTCTGC
TCATCCATGACCTGACCATGCAGAGGATGATGCTCGTGACGGTTAACGCCGCGA
ATCAGCAACGGCTTGCCGTTCAGCAGCAGCAGACCATTTTCAATCCGCACCTCGC
GGAAACCGACGTCGCAGGCTTCTGCTTCAATCAGCGTGCCGTCGGCGGTGTGCAG
TTCAACCACTGCACGATAGAGATTCGGGATTTCGGCGCTCCACAGTTCCGGATTT
TCAACGTTCAGGCGTAGTGTGACGCGATCGGCATAACCGCCACGCTCATCGATAA
TTTCACCcatgtcagccgttaagtgacctgtgtcactgaaaattgcatgagaggctctaagggcactcagtgcgttacatccctg
gcttgagtccacaaccgttaaaccttaaaagcataaaagccttatatattcattattcttataaaacttaaaaccttagaggctatttaagtt
gctgatttatattaatatattgacaaacatgagagcttagtacgtgaaacatgagagcttagtacgttagccatgagagcttagtacgttag
ccatgagggatagacgttaaacatgagagcttagtacgttaaacatgagagcttagtacgtgaaacatgagagcttagtacgtactatc
aacaggttgaactgcggatcttgcggccgcaaaaattaaaaatgaagattaaatcaatctaaagtatatatgagtaaacaggtctgaca
gTTACCAATGCTTAATCAGTGAGGCACCTATCTCAGCGATCTGTCTATTTCGTTCA
TCCATAGTTGCCTGACTCCCCGTCGTGTAGATAACTACGATACGGGAGGGCTTAC
CATCTGGCCCCAGTGCTGCAATGATACCGCGAGACCCACGCTCACCGGCTCCAG
ATTTATCAGCAATAAACCAGCCAGCCGGAAGGGCCGAGCGCAGAAGTGGTCCTG
CAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGATCAA
GGCGAGTTACATGATCCCCCATGTTGTGCAAAAAAGCGGTTAGCTCCTTCGGTCC
TCCGATCGTTGTCAGAAGTAAGTTGGCCGCAGTGTTATCACTCATGGTTATGGCA
GCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCT
TGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGT
TGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAA
AAGGGAATAAGGGCGACACGGAAATGTTGAATACTCAtactcaccatttcaatattattgaagcatt
tatcagggttattgtctcatgagcggatacatatttgaatgtatttagaaaaataaacaaataggggttccgcgACTGACGGGCT
CCAGGAGTCGTCGCCACCAATCCCCATATGGAAACCGTCGATATTCAGCCATGTG
CCTTCTTCCGCGTGCAGCAGATGGCGATGGCTGGTTTCCATCAGTTGTTGTTGGCT
GTAGCGGCTGATGTTGAACTGGAAGTCGCCGCGCCACTGGTGTGGGCCATAATTC
AATTCGCGCGTCCCGCAGCGCAGACCGTTTTCGCTCGGGAAGACGTACGGGGTAT
ACATGTCTGACAATGGCAGATCCCAGCGGTCAAAACAGGCTGCAGTAAGGCGGT
CGGGATAGTTTTCTTGCGGCCCCAGGCCGAGCCAGTTTACCCGCTCTGAGACCTG
CGCCAGCTGGCAGGTCAGGCCAATCCGCGCCGGATGCGGTGTATCGCTTGCCAC
CGCAACATCCACATTGATGACCATCTCACCGTGCCCATCAATCCGGTAGGTTTTC
CGGCTGATAAATAAGGTTTTCCCCTGATGCTGCCACGCGTGGGCGGTTGTAATCA
GCACCGCGTCGGCAAGTGTATCTGCCGTGCACTGCAACAACGCCGCTTCGGCCTG
GTAATGGCCCGCCGCCTTCCAGCGTTCGACCCAGGCGTTAGGGTCAATGCGGGTC
GCTTCACTTACGCCAATGTCGTTATCCAGCGGCGCACGGGTGAACTGATCGCGCA
GCGGGGTCAGCAGTTGTTTTTCATCGCCAATCCACATCTGTGAAAGAAAGCCTGA
CTGGCGGTTAAATTGCCAACGCTTATTACCCAGCTCGATGCAAAAATCCGTTCCG
CTGGTGGTCAGTTGAGGGATGGCGTGGGACGCGGAGGGGAGTGTCACGCTGAGG
TTTTCCGCCAGACGCCATTGCTGCCAGGCGCTGATGTGTCCGGCTTCTGACCATG
CGGTCGCGTTTGGTTGCACTACGCGTACCGTTAGCCAGAGTcacataccccgaaaagtgccac
ctgcatcgatggccccccagtgaattcgTTAAGACCCACTTTCACATTTAAGTTGTTTTTCTAATCC
GCATATGATCAATTCAAGGCCGAATAAGAAGGCTGGCTCTGCACCTTGGTGATCA
AATAATTCGATAGCTTGTCGTAATAATGGCGGCATACTATCAGTAGTAGGTGTTT
CCCTTTCTTCTTTAGCGACTTGATGCTCTTGATCTTCCAATACGCAACCTAAAGTA
AAATGCCCCACAGCGCTGAGTGCATATAATGCATTCTCTAGTGAAAAACCTTGTT
GGCATAAAAAGGCTAATTGATTTTCGAGAGTTTCATACTGTTTTTCTGTAGGCCG
TGTACCTAAATGTACTTTTGCTCCATCGCGATGACTTAGTAAAGCACATCTAAAA
CTTTTAGCGTTATTACGTAAAAAATCTTGCCAGCTTTCCCCTTCTAAAGGGCAAA
AGTGAGTATGGTGCCTATCTAACATCTCAATGGCTAAGGCGTCGAGCAAAGCCC
GCTTATTTTTTACATGCCAATACAATGTAGGCTGCTCTACACCTAGCTTCTGGGCG
AGTTTACGGGTTGTTAAACCTTCGATTCCGACCTCATTAAGCAGCTCTAATGCGC
TGTTAATCACTTTACTTTTATCTAATCTAGACATcattaattcctaatattgagacactctatcattgatag
agttatataccactccctatcagtgatagagaaaagtgaactctagaaataattagataactttaagaaggagatatacatATGAAA
CGGAATGCGAAAACTATCATCGCAGGGATGATTGCACTGGCAATTTCACACACC
GCTATGGCTGACGATATTAAAGTCGCCGTTGTCGGCGCGATGTCCGGCCCGATTG
CCCAGTGGGGCGATATGGAATTTAACGGCGCGCGTCAGGCAATTAAAGACATTA
ATGCCAAAGGGGGAATTAAGGGCGATAAACTGGTTGGCGTGGAATATGACGACG
CATGCGACCCGAAACAAGCCGTTGCGGTCGCCAACAAAATCGTTAATGACGGCA
TTAAATACGTTATTGGTCATCTGTGTTCTTCTTCTACCCAGCCTGCGTCAGATATC
TATGAAGACGAAGGTATTCTGATGATCTCGCCGGGAGCGACCAACCCGGAGCTG
ACCCAACGCGGTTATCAACACATTATGCGTACTGCCGGGCTGGACTCTTCCCAGG
GGCCAACGGCGGCAAAATACATTCTTGAGACGGTGAAGCCCCAGCGCATCGCCA
TCATTCACGACAAACAACAGTATGGCGAAGGGCTGGCGCGTTCGGTGCAGGACG
GGCTGAAAGCGGCTAACGCCAACGTCGTCTTCTTCGACGGTATTACCGCCGGGGA
GAAAGATTTCTCCGCGCTGATCGCCCGCCTGAAAAAAGAAAACATCGACTTCGTT
TACTACGGCGGTTACTACCCGGAAATGGGGCAGATGCTGCGCCAGGCCCGTTCC
GTTGGCCTGAAAACCCAGTTTATGGGGCCGGAAGGTGTGGGTAATGCGTCGTTGT
CGAACATTGCCGGTGATGCCGCCGAAGGCATGTTGGTCACTATGCCAAAACGCT
ATGACCAGGATCCGGCAAACCAGGGCATCGTTGATGCGCTGAAAGCAGACAAGA
AAGATCCGTCCGGGCCTTATGTCTGGATCACCTACGCGGCGGTGCAATCTCTGGC
GACTGCCCTTGAGCGTACCGGCAGCGATGAGCCGCTGGCGCTGGTGAAAGATTT
AAAAGCTAACGGTGCAAACACCGTGATTGGGCCGCTGAACTGGGATGAAAAAGG
CGATCTTAAGGGATTTGATTTTGGTGTCTTCCAGTGGCACGCCGACGGTTCATCC
ACGGCAGCCAAGTGAtcatcccaccgcccgtaaaatgcgggcgggtttagaaaggttaccttATGTCTGAGC
AGTTTTTGTATTTCTTGCAGCAGATGTTTAACGGCGTCACGCTGGGCAGTACCTA
CGCGCTGATAGCCATCGGCTACACCATGGTTTACGGCATTATCGGCATGATCAAC
TTCGCCCACGGCGAGGTTTATATGATTGGCAGCTACGTCTCATTTATGATCATCG
CCGCGCTGATGATGATGGGCATTGATACCGGCTGGCTGCTGGTAGCTGCGGGATT
CGTCGGCGCAATCGTCATTGCCAGCGCCTACGGCTGGAGTATCGAACGGGTGGCT
TACCGCCCGGTGCGTAACTCTAAGCGCCTGATTGCACTCATCTCTGCAATCGGTA
TGTCCATCTTCCTGCAAAACTACGTCAGCCTGACCGAAGGTTCGCGCGACGTGGC
GCTGCCGAGCCTGTTTAACGGTCAGTGGGTGGTGGGGCATAGCGAAAACTTCTCT
GCCTCTATTACCACCATGCAGGCGGTGATCTGGATTGTTACCTTCCTCGCCATGCT
GGCGCTGACGATTTTCATTCGCTATTCCCGCATGGGTCGCGCGTGTCGTGCCTGC
GCGGAAGATCTGAAAATGGCGAGTCTGCTTGGCATTAACACCGACCGGGTGATT
GCGCTGACCTTTGTGATTGGCGCGGCGATGGCGGCGGTGGCGGGTGTGCTGCTCG
GTCAGTTCTACGGCGTCATTAACCCCTACATCGGCTTTATGGCCGGGATGAAAGC
CTTTACCGCGGCGGTGCTCGGTGGGATTGGCAGCATTCCGGGAGCGATGATTGGC
GGCCTGATTCTGGGGATTGCGGAGGCGCTCTCTTCTGCCTATCTGAGTACGGAAT
ATAAAGATGTGGTGTCATTCGCCCTGCTGATTCTGGTGCTGCTGGTGATGCCGAC
CGGTATTCTGGGTCGCCCGGAGGTAGAGAAAGTATGAAACCGATGCATATTGCA
ATGGCGCTGCTCTCTGCCGCGATGTTCTTTGTGCTGGCGGGCGTCTTTATGGGCGT
GCAACTGGAGCTGGATGGCACCAAACTGGTGGTCGACACGGCTTCGGATGTCCG
TTGGCAGTGGGTGTTTATCGGCACGGCGGTGGTCTTTTTCTTCCAGCTTTTGCGAC
CGGCTTTCCAGAAAGGGTTGAAAAGCGTTTCCGGACCGAAGTTTATTCTGCCCGC
CATTGATGGCTCCACGGTGAAGCAGAAACTGTTCCTCGTGGCGCTGTTGGTGCTT
GCGGTGGCGTGGCCGTTTATGGTTTCACGCGGGACGGTGGATATTGCCACCCTGA
CCATGATCTACATTATCCTCGGTCTGGGGCTGAACGTGGTTGTTGGTCTTTCTGGT
CTGCTGGTGCTGGGGTACGGCGGTTTTTACGCCATCGGCGCTTACACTTTTGCGCT
GCTCAATCACTATTACGGCTTGGGCTTCTGGACCTGCCTGCCGATTGCTGGATTA
ATGGCAGCGGCGGCGGGCTTCCTGCTCGGTTTTCCGGTGCTGCGTTTGCGCGGTG
ACTATCTGGCGATCGTTACCCTCGGTTTCGGCGAAATTGTGCGCATATTGCTGCTC
AATAACACCGAAATTACCGGCGGCCCGAACGGAATCAGTCAGATCCCGAAACCG
ACACTCTTCGGACTCGAGTTCAGCCGTACCGCTCGTGAAGGCGGCTGGGACACGT
TCAGTAATTTCTTTGGCCTGAAATACGATCCCTCCGATCGTGTCATCTTCCTCTAC
CTGGTGGCGTTGCTGCTGGTGGTGCTAAGCCTGTTTGTCATTAACCGCCTGCTGC
GGATGCCGCTGGGGCGTGCGTGGGAAGCGTTGCGTGAAGATGAAATCGCCTGCC
GTTCGCTGGGCTTAAGCCCGCGTCGTATCAAGCTGACTGCCTTTACCATAAGTGC
CGCGTTTGCCGGTTTTGCCGGAACGCTGTTTGCGGCGCGTCAGGGCTTTGTCAGC
CCGGAATCCTTCACCTTTGCCGAATCGGCGTTTGTGCTGGCGATAGTGGTGCTCG
GCGGTATGGGCTCGCAATTTGCGGTGATTCTGGCGGCAATTTTGCTGGTGGTGTC
GCGCGAGTTGATGCGTGATTTCAACGAATACAGCATGTTAATGCTCGGTGGTTTG
ATGGTGCTGATGATGATCTGGCGTCCGCAGGGCTTGCTGCCCATGACGCGCCCGC
AACTGAAGCTGAAAAACGGCGCAGCGAAAGGAGAGCAGGCATGAGTCAGCCAT
TATTATCTGTTAACGGCCTGATGATGCGCTTCGGCGGCCTGCTGGCGGTGAACAA
CGTCAATCTTGAACTGTACCCGCAGGAGATCGTCTCGTTAATCGGCCCTAACGGT
GCCGGAAAAACCACGGTTTTTAACTGTCTGACCGGATTCTACAAACCCACCGGCG
GCACCATTTTACTGCGCGATCAGCACCTGGAAGGTTTACCGGGGCAGCAAATTGC
CCGCATGGGCGTGGTGCGCACCTTCCAGCATGTGCGTCTGTTCCGTGAAATGACG
GTAATTGAAAACCTGCTGGTGGCGCAGCATCAGCAACTGAAAACCGGGCTGTTC
TCTGGCCTGTTGAAAACGCCATCCTTCCGTCGCGCCCAGAGCGAAGCGCTCGACC
GCGCCGCGACCTGGCTTGAGCGCATTGGTTTGCTGGAACACGCCAACCGTCAGG
CGAGTAACCTGGCCTATGGTGACCAGCGCCGTCTTGAGATTGCCCGCTGCATGGT
GACGCAGCCGGAGATTTTAATGCTCGACGAACCTGCGGCAGGTCTTAACCCGAA
AGAGACGAAAGAGCTGGATGAGCTGATTGCCGAACTGCGCAATCATCACAACAC
CACTATCTTGTTGATTGAACACGATATGAAGCTGGTGATGGGAATTTCGGACCGA
ATTTACGTGGTCAATCAGGGGACGCCGCTGGCAAACGGTACGCCGGAGCAGATC
CGTAATAACCCGGACGTGATCCGTGCCTATTTAGGTGAGGCATAAgATGGAAAAA
GTCATGTTGTCCTTTGACAAAGTCAGCGCCCACTACGGCAAAATCCAGGCGCTGC
ATGAGGTGAGCCTGCATATCAATCAGGGCGAGATTGTCACGCTGATTGGCGCGA
ACGGGGCGGGGAAAACCACCTTGCTCGGCACGTTATGCGGCGATCCGCGTGCCA
CCAGCGGGCGAATTGTGTTTGATGATAAAGACATTACCGACTGGCAGACAGCGA
AAATCATGCGCGAAGCGGTGGCGATTGTCCCGGAAGGGCGTCGCGTCTTCTCGC
GGATGACGGTGGAAGAGAACCTGGCGATGGGCGGTTTTTTTGCTGAACGCGACC
AGTTCCAGGAGCGCATAAAGTGGGTGTATGAGCTGTTTCCACGTCTGCATGAGCG
CCGTATTCAGCGGGCGGGCACCATGTCCGGCGGTGAACAGCAGATGCTGGCGAT
TGGTCGTGCGCTGATGAGCAACCCGCGTTTGCTACTGCTTGATGAGCCATCGCTC
GGTCTTGCGCCGATTATCATCCAGCAAATTTTCGACACCATCGAGCAGCTGCGCG
AGCAGGGGATGACTATCTTTCTCGTCGAGCAGAACGCCAACCAGGCGCTAAAGC
TGGCGGATCGCGGCTACGTGCTGGAAAACGGCCATGTAGTGCTTTCCGATACTGG
TGATGCGCTGCTGGCGAATGAAGCGGTGAGAAGTGCGTATTTAGGCGGGTAAccg
atggtagtgtggggtctccccatgcgagagtagggaactgccaggcatcaaataaaacgaaaggctcagtcgaaagactgggccat
cgattatctgagtagtcggtgaacgctctcctgagtaggacaaatccgccgggagcggatttgaacgttgcgaagcaacggcccgg
agggtggcgggcaggacgcccgccataaactgccaggcatcaaattaagcagaaggccatcctgacggatggccatttgcgtggc
cagtgccaagcttgcatgc
SEQ ID NO: 8: E. coli Nissle 1917 leucine exporter gene leuE
GTGTTCGCTGAATACGGGGTTCTGAATTACTGGACCTATCTGGTTGGGGCCATTT
TTATTGTGTTGGTGCCAGGGCCAAATACCCTGTTTGTACTCAAAAATAGCGTCAG
TAGCGGTATGAAAGGCGGTTATCTTGCGGCCTGTGGTGTATTTATTGGCGATGCG
GTATTGATGTTTCTGGCATGGGCTGGAGTGGCGACATTAATTAAGACCACCCCGA
TATTATTCAACATCGTACGTTATCTTGGTGCGTTTTATTTGCTCTATCTGGGGAGT
AAAATTCTCTACGCGACCCTGAAAGGTAAAAATAGCGAGACCAAATCCGATGAG
CCCCAATACGGTGCCATTTTTAAACGCGCGTTAATTTTGAGCCTGACTAATCCGA
AAGCCATTTTGTTCTATGTGTCGTTTTTCGTACAGTTTATCGATGTTAATGCCCCA
CATACGGGAATTTCATTCTTTATTCTGGCGACGACGCTGGAACTGGTGAGTTTCT
GCTATTTGAGCTTCCTGATTATTTCTGGGGCTTTTGTCACGCAGTACATACGTACC
AAAAAGAAACTGGCTAAAGTGGGCAACTCACTGATTGGTTTGATGTTCGTGGGTT
TCGCCGCCCGACTGGCGACGCTGCAATCCTGA
SEQ ID NO: 9: leuE deletion construct:
catataaataccatttattggttactattagcaccatatcagcgaagaatcagggaggattatagatgggaagcccatgcagattgcagc
attacacgtcttgagcgattgtgtaggctggagctgcttcgaagttcctatactttctagagaataggaacttcggaataggaacttcaag
atcccctcacgctgccgcaagcactcagggcgcaagggctgctaaaggaagcggaacacgtagaaagccagtccgcagaaacgg
tgctgaccccggatgaatgtcagctactgggctatctggacaagggaaaacgcaagcgcaaagagaaagcaggtagcttgcagtgg
gcttacatggcgatagctagactgggcggattatggacagcaagcgaaccggaattgccagctggggcgccctctggtaaggagg
gaagccctgcaaagtaaactggatggctacttgccgccaaggatctgatggcgcaggggatcaagatctgatcaagagacaggatg
aggatcgatcgcATGATTGAACAAGATGGATTGCACGCAGGTTCTCCGGCCGCTTGGGT
GGAGAGGCTATTCGGCTATGACTGGGCACAACAGACAATCGGCTGCTCTGATGC
CGCCGTGTTCCGGCTGTCAGCGCAGGGGCGCCCGGTTCTTTTTGTCAAGACCGAC
CTGTCCGGTGCCCTGAATGAACTGCAGGACGAGGCAGCGCGGCTATCGTGGCTG
GCCACGACGGGCGTTCCTTGCGCAGCTGTGCTCGACGTTGTCACTGAAGCGGGAA
GGGACTGGCTGCTATTGGGCGAAGTGCCGGGGCAGGATCTCCTGTCATCTCACCT
TGCTCCTGCCGAGAAAGTATCCATCATGGCTGATGCAATGCGGCGGCTGCATACG
CTTGATCCGGCTACCTGCCCATTCGACCACCAAGCGAAACATCGCATCGAGCGA
GCACGTACTCGGATGGAAGCCGGTCTTGTCGATCAGGATGATCTGGACGAAGAG
CATCAGGGGCTCGCGCCAGCCGAACTGTTCGCCAGGCTCAAGGCGCGCATGCCC
GACGGCGAGGATCTCGTCGTGACCCATGGCGATGCCTGCTTGCCGAATATCATGG
TGGAAAATGGCCGCTTTTCTGGATTCATCGACTGTGGCCGGCTGGGTGTGGCGGA
CCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGC
GCATCGCCTTCTATCGCCTTCTTGACGAGTTCTTCTGAgcgggactctggggacgaaatgaccga
ccaagcgacgcccaacctgccatcacgagatttcgattccaccgccgccactatgaaaggagggcttcggaatcgattccgggacg
ccggctggatgatcctccagcgcggggatctcatgctggagacttcgcccaccccagcttcaaaagcgctctgaagacctatactact
agagaataggaacttcggaataggaactaaggaggatattcatatggaccatggctaattcccaattaacctattaattatattcgatcat
gcgcgattaaaggtgaatatgctaaccaatctgtagcggcttagaaaggagaaaatcaggattaacctga
SEQ ID NO: 10: Tet-livKHMGF fragment
aataggggaccgcgACTGACGGGCTCCAGGAGTCGTCGCCACCAATCCCCATATGGAAA
CCGTCGATATTCAGCCATGTGCCTTCTTCCGCGTGCAGCAGATGGCGATGGCTGG
TTTCCATCAGTTGTTGTTGGCTGTAGCGGCTGATGTTGAACTGGAAGTCGCCGCG
CCACTGGTGTGGGCCATAATTCAATTCGCGCGTCCCGCAGCGCAGACCGTTTTCG
CTCGGGAAGACGTACGGGGTATACATGTCTGACAATGGCAGATCCCAGCGGTCA
AAACAGGCTGCAGTAAGGCGGTCGGGATAGTTTTCTTGCGGCCCCAGGCCGAGC
CAGTTTACCCGCTCTGAGACCTGCGCCAGCTGGCAGGTCAGGCCAATCCGCGCCG
GATGCGGTGTATCGCTTGCCACCGCAACATCCACATTGATGACCATCTCACCGTG
CCCATCAATCCGGTAGGTTTTCCGGCTGATAAATAAGGTTTTCCCCTGATGCTGC
CACGCGTGGGCGGTTGTAATCAGCACCGCGTCGGCAAGTGTATCTGCCGTGCACT
GCAACAACGCCGCTTCGGCCTGGTAATGGCCCGCCGCCTTCCAGCGTTCGACCCA
GGCGTTAGGGTCAATGCGGGTCGCTTCACTTACGCCAATGTCGTTATCCAGCGGC
GCACGGGTGAACTGATCGCGCAGCGGGGTCAGCAGTTGTTTTTCATCGCCAATCC
ACATCTGTGAAAGAAAGCCTGACTGGCGGTTAAATTGCCAACGCTTATTACCCAG
CTCGATGCAAAAATCCGTTCCGCTGGTGGTCAGTTGAGGGATGGCGTGGGACGC
GGAGGGGAGTGTCACGCTGAGGTTTTCCGCCAGACGCCATTGCTGCCAGGCGCT
GATGTGTCCGGCTTCTGACCATGCGGTCGCGTTTGGTTGCACTACGCGTACCGTT
AGCCAGAGTcacataccccgaaaagtgccacctgcatcgatggccccccagtgaattcgTTAAGACCCACTTT
CACATTTAAGTTGTTTTTCTAATCCGCATATGATCAATTCAAGGCCGAATAAGAA
GGCTGGCTCTGCACCTTGGTGATCAAATAATTCGATAGCTTGTCGTAATAATGGC
GGCATACTATCAGTAGTAGGTGTTTCCCTTTCTTCTTTAGCGACTTGATGCTCTTG
ATCTTCCAATACGCAACCTAAAGTAAAATGCCCCACAGCGCTGAGTGCATATAAT
GCATTCTCTAGTGAAAAACCTTGTTGGCATAAAAAGGCTAATTGATTTTCGAGAG
TTTCATACTGTTTTTCTGTAGGCCGTGTACCTAAATGTACTTTTGCTCCATCGCGA
TGACTTAGTAAAGCACATCTAAAACTTTTAGCGTTATTACGTAAAAAATCTTGCC
AGCTTTCCCCTTCTAAAGGGCAAAAGTGAGTATGGTGCCTATCTAACATCTCAAT
GGCTAAGGCGTCGAGCAAAGCCCGCTTATTTTTTACATGCCAATACAATGTAGGC
TGCTCTACACCTAGCTTCTGGGCGAGTTTACGGGTTGTTAAACCTTCGATTCCGAC
CTCATTAAGCAGCTCTAATGCGCTGTTAATCACTTTACTTTTATCTAATCTAGACA
Tcattaattcctaatattgagacactctatcattgatagagttatataccactccctatcagtgatagagaaaagtgaactctagaaataat
tttgtttaactttaagaaggagatatacatATGAAACGGAATGCGAAAACTATCATCGCAGGGATGA
TTGCACTGGCAATTTCACACACCGCTATGGCTGACGATATTAAAGTCGCCGTTGT
CGGCGCGATGTCCGGCCCGATTGCCCAGTGGGGCGATATGGAATTTAACGGCGC
GCGTCAGGCAATTAAAGACATTAATGCCAAAGGGGGAATTAAGGGCGATAAACT
GGTTGGCGTGGAATATGACGACGCATGCGACCCGAAACAAGCCGTTGCGGTCGC
CAACAAAATCGTTAATGACGGCATTAAATACGTTATTGGTCATCTGTGTTCTTCTT
CTACCCAGCCTGCGTCAGATATCTATGAAGACGAAGGTATTCTGATGATCTCGCC
GGGAGCGACCAACCCGGAGCTGACCCAACGCGGTTATCAACACATTATGCGTAC
TGCCGGGCTGGACTCTTCCCAGGGGCCAACGGCGGCAAAATACATTCTTGAGAC
GGTGAAGCCCCAGCGCATCGCCATCATTCACGACAAACAACAGTATGGCGAAGG
GCTGGCGCGTTCGGTGCAGGACGGGCTGAAAGCGGCTAACGCCAACGTCGTCTT
CTTCGACGGTATTACCGCCGGGGAGAAAGATTTCTCCGCGCTGATCGCCCGCCTG
AAAAAAGAAAACATCGACTTCGTTTACTACGGCGGTTACTACCCGGAAATGGGG
CAGATGCTGCGCCAGGCCCGTTCCGTTGGCCTGAAAACCCAGTTTATGGGGCCGG
AAGGTGTGGGTAATGCGTCGTTGTCGAACATTGCCGGTGATGCCGCCGAAGGCA
TGTTGGTCACTATGCCAAAACGCTATGACCAGGATCCGGCAAACCAGGGCATCG
TTGATGCGCTGAAAGCAGACAAGAAAGATCCGTCCGGGCCTTATGTCTGGATCA
CCTACGCGGCGGTGCAATCTCTGGCGACTGCCCTTGAGCGTACCGGCAGCGATG
AGCCGCTGGCGCTGGTGAAAGATTTAAAAGCTAACGGTGCAAACACCGTGATTG
GGCCGCTGAACTGGGATGAAAAAGGCGATCTTAAGGGATTTGATTTTGGTGTCTT
CCAGTGGCACGCCGACGGTTCATCCACGGCAGCCAAGTGAtcatcccaccgcccgtaaaatgc
gggcgggatagaaaggttaccttATGTCTGAGCAGTTTTTGTATTTCTTGCAGCAGATGTTTAA
CGGCGTCACGCTGGGCAGTACCTACGCGCTGATAGCCATCGGCTACACCATGGTT
TACGGCATTATCGGCATGATCAACTTCGCCCACGGCGAGGTTTATATGATTGGCA
GCTACGTCTCATTTATGATCATCGCCGCGCTGATGATGATGGGCATTGATACCGG
CTGGCTGCTGGTAGCTGCGGGATTCGTCGGCGCAATCGTCATTGCCAGCGCCTAC
GGCTGGAGTATCGAACGGGTGGCTTACCGCCCGGTGCGTAACTCTAAGCGCCTG
ATTGCACTCATCTCTGCAATCGGTATGTCCATCTTCCTGCAAAACTACGTCAGCCT
GACCGAAGGTTCGCGCGACGTGGCGCTGCCGAGCCTGTTTAACGGTCAGTGGGT
GGTGGGGCATAGCGAAAACTTCTCTGCCTCTATTACCACCATGCAGGCGGTGATC
TGGATTGTTACCTTCCTCGCCATGCTGGCGCTGACGATTTTCATTCGCTATTCCCG
CATGGGTCGCGCGTGTCGTGCCTGCGCGGAAGATCTGAAAATGGCGAGTCTGCTT
GGCATTAACACCGACCGGGTGATTGCGCTGACCTTTGTGATTGGCGCGGCGATGG
CGGCGGTGGCGGGTGTGCTGCTCGGTCAGTTCTACGGCGTCATTAACCCCTACAT
CGGCTTTATGGCCGGGATGAAAGCCTTTACCGCGGCGGTGCTCGGTGGGATTGGC
AGCATTCCGGGAGCGATGATTGGCGGCCTGATTCTGGGGATTGCGGAGGCGCTCT
CTTCTGCCTATCTGAGTACGGAATATAAAGATGTGGTGTCATTCGCCCTGCTGAT
TCTGGTGCTGCTGGTGATGCCGACCGGTATTCTGGGTCGCCCGGAGGTAGAGAAA
GTATGAAACCGATGCATATTGCAATGGCGCTGCTCTCTGCCGCGATGTTCTTTGT
GCTGGCGGGCGTCTTTATGGGCGTGCAACTGGAGCTGGATGGCACCAAACTGGT
GGTCGACACGGCTTCGGATGTCCGTTGGCAGTGGGTGTTTATCGGCACGGCGGTG
GTCTTTTTCTTCCAGCTTTTGCGACCGGCTTTCCAGAAAGGGTTGAAAAGCGTTTC
CGGACCGAAGTTTATTCTGCCCGCCATTGATGGCTCCACGGTGAAGCAGAAACTG
TTCCTCGTGGCGCTGTTGGTGCTTGCGGTGGCGTGGCCGTTTATGGTTTCACGCGG
GACGGTGGATATTGCCACCCTGACCATGATCTACATTATCCTCGGTCTGGGGCTG
AACGTGGTTGTTGGTCTTTCTGGTCTGCTGGTGCTGGGGTACGGCGGTTTTTACGC
CATCGGCGCTTACACTTTTGCGCTGCTCAATCACTATTACGGCTTGGGCTTCTGGA
CCTGCCTGCCGATTGCTGGATTAATGGCAGCGGCGGCGGGCTTCCTGCTCGGTTT
TCCGGTGCTGCGTTTGCGCGGTGACTATCTGGCGATCGTTACCCTCGGTTTCGGC
GAAATTGTGCGCATATTGCTGCTCAATAACACCGAAATTACCGGCGGCCCGAAC
GGAATCAGTCAGATCCCGAAACCGACACTCTTCGGACTCGAGTTCAGCCGTACC
GCTCGTGAAGGCGGCTGGGACACGTTCAGTAATTTCTTTGGCCTGAAATACGATC
CCTCCGATCGTGTCATCTTCCTCTACCTGGTGGCGTTGCTGCTGGTGGTGCTAAGC
CTGTTTGTCATTAACCGCCTGCTGCGGATGCCGCTGGGGCGTGCGTGGGAAGCGT
TGCGTGAAGATGAAATCGCCTGCCGTTCGCTGGGCTTAAGCCCGCGTCGTATCAA
GCTGACTGCCTTTACCATAAGTGCCGCGTTTGCCGGTTTTGCCGGAACGCTGTTTG
CGGCGCGTCAGGGCTTTGTCAGCCCGGAATCCTTCACCTTTGCCGAATCGGCGTT
TGTGCTGGCGATAGTGGTGCTCGGCGGTATGGGCTCGCAATTTGCGGTGATTCTG
GCGGCAATTTTGCTGGTGGTGTCGCGCGAGTTGATGCGTGATTTCAACGAATACA
GCATGTTAATGCTCGGTGGTTTGATGGTGCTGATGATGATCTGGCGTCCGCAGGG
CTTGCTGCCCATGACGCGCCCGCAACTGAAGCTGAAAAACGGCGCAGCGAAAGG
AGAGCAGGCATGAGTCAGCCATTATTATCTGTTAACGGCCTGATGATGCGCTTCG
GCGGCCTGCTGGCGGTGAACAACGTCAATCTTGAACTGTACCCGCAGGAGATCG
TCTCGTTAATCGGCCCTAACGGTGCCGGAAAAACCACGGTTTTTAACTGTCTGAC
CGGATTCTACAAACCCACCGGCGGCACCATTTTACTGCGCGATCAGCACCTGGAA
GGTTTACCGGGGCAGCAAATTGCCCGCATGGGCGTGGTGCGCACCTTCCAGCATG
TGCGTCTGTTCCGTGAAATGACGGTAATTGAAAACCTGCTGGTGGCGCAGCATCA
GCAACTGAAAACCGGGCTGTTCTCTGGCCTGTTGAAAACGCCATCCTTCCGTCGC
GCCCAGAGCGAAGCGCTCGACCGCGCCGCGACCTGGCTTGAGCGCATTGGTTTG
CTGGAACACGCCAACCGTCAGGCGAGTAACCTGGCCTATGGTGACCAGCGCCGT
CTTGAGATTGCCCGCTGCATGGTGACGCAGCCGGAGATTTTAATGCTCGACGAAC
CTGCGGCAGGTCTTAACCCGAAAGAGACGAAAGAGCTGGATGAGCTGATTGCCG
AACTGCGCAATCATCACAACACCACTATCTTGTTGATTGAACACGATATGAAGCT
GGTGATGGGAATTTCGGACCGAATTTACGTGGTCAATCAGGGGACGCCGCTGGC
AAACGGTACGCCGGAGCAGATCCGTAATAACCCGGACGTGATCCGTGCCTATTT
AGGTGAGGCATAAgATGGAAAAAGTCATGTTGTCCTTTGACAAAGTCAGCGCCCA
CTACGGCAAAATCCAGGCGCTGCATGAGGTGAGCCTGCATATCAATCAGGGCGA
GATTGTCACGCTGATTGGCGCGAACGGGGCGGGGAAAACCACCTTGCTCGGCAC
GTTATGCGGCGATCCGCGTGCCACCAGCGGGCGAATTGTGTTTGATGATAAAGAC
ATTACCGACTGGCAGACAGCGAAAATCATGCGCGAAGCGGTGGCGATTGTCCCG
GAAGGGCGTCGCGTCTTCTCGCGGATGACGGTGGAAGAGAACCTGGCGATGGGC
GGTTTTTTTGCTGAACGCGACCAGTTCCAGGAGCGCATAAAGTGGGTGTATGAGC
TGTTTCCACGTCTGCATGAGCGCCGTATTCAGCGGGCGGGCACCATGTCCGGCGG
TGAACAGCAGATGCTGGCGATTGGTCGTGCGCTGATGAGCAACCCGCGTTTGCTA
CTGCTTGATGAGCCATCGCTCGGTCTTGCGCCGATTATCATCCAGCAAATTTTCG
ACACCATCGAGCAGCTGCGCGAGCAGGGGATGACTATCTTTCTCGTCGAGCAGA
ACGCCAACCAGGCGCTAAAGCTGGCGGATCGCGGCTACGTGCTGGAAAACGGCC
ATGTAGTGCTTTCCGATACTGGTGATGCGCTGCTGGCGAATGAAGCGGTGAGAA
GTGCGTATTTAGGCGGGTAAccgatggtagtgtggggtctccccatgcgagagtagggaactgccaggcatcaaa
taaaacgaaaggctcagtcgaaagactgggccatcgattatctgagtagtcggtgaacgctctcctgagtaggacaaatccgccgg
gagcggatttgaacgagcgaagcaacggcccggagggtggcgggcaggacgcccgccataaactgccaggcatcaaattaagca
gaaggccatcctgacggatggccatttgcgtggccagtgccaagcttgcatgcagattgcagcattacacgtcttgagcgattgtgtag
gctggagctgcttcgaagacctatactactagagaataggaacttcggaataggaacttcatttaaatggcgcgccttacgccccgccc
tgccacTCATCGCAGTACTGTTGTATTCATTAAGCATCTGCCGACATGGAAGCCATC
ACAAACGGCATGATGAACCTGAATCGCCAGCGGCATCAGCACCTTGTCGCCTTG
CGTATAATATTTGCCCATGGTGAAAACGGGGGCGAAGAAGTTGTCCATATTGGCC
ACGTTTAAATCAAAACTGGTGAAACTCACCCAGGGATTGGCTGAGACGAAAAAC
ATATTCTCAATAAACCCTTTAGGGAAATAGGCCAGGTTTTCACCGTAACACGCCA
CATCTTGCGAATATATGTGTAGAAACTGCCGGAAATCGTCGTGGTATTCACTCCA
GAGCGATGAAAACGTTTCAGTTTGCTCATGGAAAACGGTGTAACAAGGGTGAAC
ACTATCCCATATCACCAGCTCACCGTCTTTCATTGCCATACGTAATTCCGGATGA
GCATTCATCAGGCGGGCAAGAATGTGAATAAAGGCCGGATAAAACTTGTGCTTA
TTTTTCTTTACGGTCTTTAAAAAGGCCGTAATATCCAGCTGAACGGTCTGGTTATA
GGTACATTGAGCAACTGACTGAAATGCCTCAAAATGTTCTTTACGATGCCATTGG
GATATATCAACGGTGGTATATCCAGTGATTTTTTTCTCCATtttagcttccttagctcctgaaaatc
tcgacaactcaaaaaatacgcccggtagtgatcttatttcattatggtgaaagaggaacctcttacgtgccgatcaacgtctcattacgcc
aaaagaggcccagggcacccggtatcaacagggacaccaggatttatttattctgcgaagtgatcaccgtcacaggtaggcgcgcc
gaagacctatactactagagaataggaacttcggaataggaactaaggaggatattcatatggaccatggctaattccTTGCCGT
TTTCATCATATTTAATCAGCGACTGATCCACCCAGTCCCAGACGAAGCCGCCCTG
TAAACGGGGGTACTGACGAAACGCCTGCCAGTATTTAGCGAAGCCGCCAAGACT
GTTACCCATCGCGTGGGCATATTCGCAAAGGATCAGCGGGCGCATTTCTCCAGGC
AGCGAAAGCCATTTTTTGATGGACCATTTCGGCACCGCCGGGAAGGGCTGGTCTT
CATCCACGCGCGCGTACATCGGGCAAATAATATCGGTGGCCGTGGTGTCGGCTCC
GCCGCCTTCATACTGTACCGGGCGGGAAGGATCGACAGATTTGATCCAGCGATA
CAGCGCGTCGTGATTAGCGCCGTGGCCTGATTCATTCCCCAGCGACCAGATGATC
ACACTCGGGTGATTACGATCGCGCTGCACCATCCGCGTTACGCGTTCGCTCATCG
CGGGTAGCCAGCGCGGATCATCGGTCAGACGATTCATTGGCACCATGCCGTGGG
TTTCAATATTGGCTTCATCCACCACATACAGGCCGTAGCGGTCGCACAGCGTGTA
CCACAGCGGATGGTTCGGATAATGCGAACAGCGCACGGCGTTAAAGTTGTTCTG
CTTCATCAGCAGGATATCCTGCACCATCGTCTGCTCATCCATGACCTGACCATGC
AGAGGATGATGCTCGTGACGGTTAACGCCGCGAATCAGCAACGGCTTGCCGTTC
AGCAGCAGCAGACCATTTTCAATCCGCACCTCGCGGAAACCGACGTCGCAGGCT
TCTGCTTCAATCAGCGTGCCGTCGGCGGTGTGCAGTTCAACCACTGCACGATAGA
GATTCGGGATTTCGGCGCTCCACAGTTCCGGATTTTCAACGTTCAGGCGTAGTGT
GACGCGATCGGCATAACCGCCACGCTCATCGATAATTTCACCcatgtcagccgttaag
SEQ ID NO: 12-livJ sequence
ATGAACATAAAGGGTAAAGCGTTACTGGCAGGATGTATCGCGCTGGCATTCAGC
AATATGGCTCTGGCAGAAGATATTAAAGTCGCGGTCGTGGGCGCAATGTCCGGT
CCGGTTGCGCAGTACGGTGACCAGGAGTTTACCGGCGCAGAGCAGGCGGTTGCG
GATATCAACGCTAAAGGCGGCATTAAAGGCAACAAACTGCAAATCGTAAAATAT
GACGATGCCTGTGACCCGAAACAGGCGGTTGCGGTGGCGAACAAAGTCGTTAAC
GACGGCATTAAATATGTGATTGGTCACCTCTGTTCTTCATCAACGCAGCCTGCGT
CTGACATCTACGAAGACGAAGGCATTTTAATGATCACCCCAGCGGCAACCGCGC
CGGAGCTGACCGCCCGTGGCTATCAGCTGATCCTGCGCACCACCGGCCTGGACTC
CGACCAGGGGCCGACGGCGGCGAAATATATTCTTGAGAAAGTGAAACCGCAGCG
TATTGCTATCGTTCACGACAAACAGCAATACGGCGAAGGTCTGGCGCGAGCGGT
GCAGGACGGCCTGAAGAAAGGCAATGCAAACGTGGTGTTCTTTGATGGCATCAC
CGCCGGGGAAAAAGATTTCTCAACGCTGGTGGCGCGTCTGAAAAAAGAGAATAT
CGACTTCGTTTACTACGGCGGTTATCACCCGGAAATGGGGCAAATCCTGCGTCAG
GCACGCGCGGCAGGGCTGAAAACTCAGTTTATGGGGCCGGAAGGTGTGGCTAAC
GTTTCGCTGTCTAACATTGCGGGCGAATCAGCGGAAGGGCTGCTGGTGACCAAG
CCGAAGAACTACGATCAGGTTCCGGCGAACAAACCCATTGTTGACGCGATCAAA
GCGAAAAAACAGGACCCAAGTGGCGCATTCGTTTGGACCACCTACGCCGCGCTG
CAATCTTTGCAGGCGGGCCTGAATCAGTCTGACGATCCGGCTGAAATCGCCAAAT
ACCTGAAAGCGAACTCCGTGGATACCGTAATGGGACCGCTGACCTGGGATGAGA
AAGGCGATCTGAAAGGCTTTGAGTTCGGCGTATTTGACTGGCACGCCAACGGCA
CGGCGACCGATGCGAAGTAA
SEQ ID NO: 11: Ptac-livJ construct
AGACAACAAGTCCACGTTGCAGGAACTGGCTGACCGTTACGGTGTTTCCGCTGAG
CGTGTGCGTCAGCTGGAAAAGAACGCGATGAAAAAATTGCGCGCTGCCATTGAA
GCGTAAtaccgctattaagcagagaaccctggatgagagtccggggtattgattagggcctctacaataatcaattccccctccg
gcaaaacgccaatccccacgcagattgttaataaactgtcaaaatagctataacacataccccgaaaagtgccgatggccccccgatg
gtagtgtggcccatgcgagagtagggaactgccaggcatcaaataaaacgaaaggctcagtcgaaagactgggccatcgattatct
gagtagtcggtgaacgctctcctgagtaggacaaatccgccgggagcggatttgaacgttgcgaagcaacggcccggagggtggc
gggcaggacgcccgccataaactgccaggcatcaaattaagcagaaggccatcctgacggatggccatttgcgtggccagtgccaa
gcttgcatgcagattgcagcattacacgtcttgagcgattgtgtaggctggagctgcttcgaagacctatactactagagaataggaact
tcggaataggaacttcaagatcccctcacgctgccgcaagcactcagggcgcaagggctgctaaaggaagcggaacacgtagaaa
gccagtccgcagaaacggtgctgaccccggatgaatgtcagctactgggctatctggacaagggaaaacgcaagcgcaaagagaa
agcaggtagcttgcagtgggcttacatggcgatagctagactgggcggattatggacagcaagcgaaccggaattgccagctgggg
cgccctctggtaaggagggaagccctgcaaagtaaactggatggctucttgccgccaaggatctgatggcgcaggggatcaagatc
tgatcaagagacaggatgaggatcgatcgcATGATTGAACAAGATGGATTGCACGCAGGTTCTCC
GGCCGCTTGGGTGGAGAGGCTATTCGGCTATGACTGGGCACAACAGACAATCGG
CTGCTCTGATGCCGCCGTGTTCCGGCTGTCAGCGCAGGGGCGCCCGGTTCTTTTT
GTCAAGACCGACCTGTCCGGTGCCCTGAATGAACTGCAGGACGAGGCAGCGCGG
CTATCGTGGCTGGCCACGACGGGCGTTCCTTGCGCAGCTGTGCTCGACGTTGTCA
CTGAAGCGGGAAGGGACTGGCTGCTATTGGGCGAAGTGCCGGGGCAGGATCTCC
TGTCATCTCACCTTGCTCCTGCCGAGAAAGTATCCATCATGGCTGATGCAATGCG
GCGGCTGCATACGCTTGATCCGGCTACCTGCCCATTCGACCACCAAGCGAAACAT
CGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAG
GCGCGCATGCCCGACGGCGAGGATCTCGTCGTGACCCATGGCGATGCCTGCTTGC
CGAATATCATGGTGGAAAATGGCCGCTTTTCTGGATTCATCGACTGTGGCCGGCT
GGGTGTGGCGGACCGCTATCAGGACATAGCGTTGGCTACCCGTGATATTGCTGAA
GAGCTTGGCGGCGAATGGGCTGACCGCTTCCTCGTGCTTTACGGTATCGCCGCTC
CCGATTCGCAGCGCATCGCCTTCTATCGCCTTCTTGACGAGTTCTTCTGAgcgggactc
tggggacgaaatgaccgaccaagcgacgcccaacctgccatcacgagatttcgattccaccgccgccactatgaaaggagggcttc
ggaatcgattccgggacgccggctggatgatcctccagcgcggggatctcatgctggagacttcgcccaccccagcttcaaaagcg
ctctgaagacctatactactagagaataggaacttcggaataggaactaaggaggatattcatatggaccatggctaattcccatgaga
caattaatcatcggctcgtataatgttagcagagtatgctgctaaagcacgggtagctacgtataaaacgaaataaagtgctgcacaaca
acatcacaacacacgtaataaccagaagagtggggattctcaggATGAACATAAAGGGTAAAGCGTTACTG
GCAGGATGTATCGCGCTGGCATTCAGCAATATGGCTCTGGCAGAAGATATTAAA
GTCGCCGTCGTAGGCGCAATGTCCGGTCCGGTGGCGCAG
SEQ ID NO: 13-Prp promoter (prpR sequence-underlined; Ribosome binding site-
lower case; start codon of gene of interest (italicized atg)
TTACCCGTCTGGATTTTCAGTACGCGCTTTTAAACGACGCCACAGCGTGGTACGGCTGATCCC
CAAATAACGTGCGGCGGCGCGCTTATCGCCATTAAAGCGTGCGAGCACCTCCTGCAATGGAAG
CGCTTCTGCTGACGAGGGCGTGATTTCTGCTGTGGTCCCCACCAGTTCAGGTAATAATTGCCG
CATAAATTGTCTGTCCAGTGTTGGTGCGGGATCGACGCTTAAAAAAAGCGCCAGGCGTTCCA
TCATATTCCGCAGTTCGCGAATATTACCGGGCCAATGATAGTTCAGTAGAAGCGGCTGACAC
TGCGTCAGCCCATGACGCACCGATTCGGTAAAAGGGATCTCCATCGCGGCCAGCGATTGTTTT
AAAAAGTTTTCCGCCAGAGGCAGAATATCAGGCTGTCGCTCGCGCAAGGGGGGAAGCGGCAG
ACGCAGAATGCTCAAACGGTAAAACAGATCGGTACGAAAACGTCCTTGCGTTATCTCCCGAT
CCAGATCGCAATGCGTGGCGCTGATCACCCGGACATCTACCGGGATCGGCTGATGCCCGCCAA
CGCGGGTGACGGCTTTTTCCTCCAGTACGCGTAGAAGGCGGGTTTGTAACGGCAGCGGCATTT
CGCCAATTTCGTCAAGAAACAGCGTGCCGCCGTGGGCGACCTCAAACAGCCCCGCACGTCCAC
CTCGTCTTGAGCCGGTAAACGCTCCCTCCTCATAGCCAAACAGTTCAGCCTCCAGCAACGACT
CGGTAATCGCGCCGCAATTAACGGCGACAAAGGGCGGAGAAGGCTTGTTCTGACGGTGGGGC
TGACGGTTAAACAACGCCTGATGAATCGCTTGCGCCGCCAGCTCTTTCCCGGTCCCTGTTTCC
CCCTGAATCAGCACTGCCGCGCGGGAACGGGCATAGAGTGTAATCGTATGGCGAACCTGCTCC
ATTTGTGGTGAATCGCCGAGGATATCGCTCAGCGCATAACGGGTCTGTAATCCCTTGCTGGA
GGTATGCTGGCTATACTGACGCCGTGTCAGGCGGGTCATATCCAGCGCATCATGGAAAGCCT
GACGTACGGTGGCCGCTGAATAAATAAAGATGGCGGTCATTCCTGCCTCTTCCGCCAGGTCGG
TAATTAGTCCTGCCCCAATTACAGCCTCAATGCCGTTAGCTTTGAGCTCGTTAATTTGCCCGC
GAGCATCCTCTTCAGTGATATAGCTTCGCTGTTCAAGACGGAGGTGAAACGTTTTCTGAAAG
GCGACCAGAGCCGGAATGGTCTCCTGATAGGTCACGATTCCCATTGAGGAAGTCAGCTTTCCC
GCTTTTGCCAGAGCCTGTAATACATCGAATCCGCTGGGTTTGATGAGGATGACAGGTACCGA
CAGTCGGCTTTTTAAATAAGCGCCGTTGGAACCTGCCGCGATAATCGCGTCGCAGCGTTCGGT
TGCCAGTTTTTTGCGAATGTAGGCTACTGCCTTTTCAAAACCGAGCTGAATAGGCGTGATCG
TCGCCAGATGATCAAACTCCAGGCTGATATCCCGAAATAGTTCGAACAGGCGCGTTACCGAG
ACCGTCCAGATCACCGGTTTATCGCTATTATCGCGCGAAGCGCTATGCACAGTAACCATCGTC
GTAGATTCATGTTTAAGGAACGAATTCTTGTTTTATAGATGTTTCGTTAATGTTGCAATGAA
ACACAGGCCTCCGTTTCATGAAACGTTAGCTGACTCGTTTTTCTTGTGACTCGTCTGTCAGTA
TTAAAAAAGATTTTTCATTTAACTGATTGTTTTTAAATTGAATTTTATTTAATGGTTTCTCG
GTTTTTGGGTCTGGCATATCCCTTGCTTTAATGAGTGCATCTTAATTAACAATTCAATAACA
AGAGGGCTGAATagtaatttcaacaaaataacgagcattcgaatg
I. Enzymes catalyzing the conversion of branched-chain amino acids into
their ketoacids
1. Leucine dehydrogenase LeuDH from Pseudomonas aeruginosa PA01
SEQ ID NO: 19-LeuDH Amino acid sequence:
MFDMMDAARLEGLHLAQDPATGLKAIIAIHSTRLGPALGGCRYLPYPNDEAAIGDAI
RLAQGMSYKAALAGLEQGGGKAVIIRPPHLDNRGALFEAFGRFIESLGGRYITAVDS
GTSSADMDCIAQQTRHVTSTTQAGDPSPHTALGVFAGIRASAQARLGSDDLEGLRVA
VQGLGHVGYALAEQLAAVGAELLVCDLDPGRVQLAVEQLGAHPLAPEALLSTPCDI
LAPCGLGGVLTSQSVSQLRCAAVAGAANNQLERPEVADELEARGILYAPDYVINSGG
LIYVALKHRGADPHSITAHLARIPARLTEIYAHAQADHQSPARIADRLAERILYGPQ
SEQ ID NO: 20-leaDH codon-optimized nucleotide sequence:
atgacgacatgatggatgcagcccgcctggaaggcctgcacctcgcccaggatccagcgacgggcctgaaagcgatcatcgcgat
ccattccactcgcctcggcccggccttaggcggctgtcgttacctcccatatccgaatgatgaagcggccatcggcgatgccattcgcc
tggcgcagggcatgtcctacaaagctgcacttgcgggtctggaacaaggtggtggcaaggcggtgatcattcgcccaccccacttgg
ataatcgcggtgccttgatgaagcgtaggacgattattgaaagcctgggtggccgttatatcaccgccgttgactcaggaacaagtag
tgccgatatggattgcatcgcccaacagacgcgccatgtgacttcaacgacacaagccggcgacccatctccacatacggctctggg
cgtattgccggcatccgcgcctccgcgcaggctcgcctggggtccgatgacctggaaggcctgcgtgtcgcggttcagggccagg
ccacgtaggttatgcgttagcggagcagctggcggcggtcggcgcagaactgctggtgtgcgacctggaccccggccgcgtccagt
tagcggtggagcaactgggggcgcacccactggcccctgaagcattgctctctactccgtgcgacatatagcgccagtggcctggg
cggcgtgctcaccagccagtcggtgtcacagagcgctgcgcggccgttgcaggcgcagcgaacaatcaactggagcgcccggaa
gagcagacgaactggaggcgcgcgggatatatatgcgcccgattacgtgattaactcgggaggactgatttatgtggcgctgaagca
tcgcggtgctgatccgcatagcattaccgcccacctcgctcgcatccctgcacgcctgacggaaatctatgcgcatgcgcaggcggat
catcagtcgcctgcgcgcatcgccgatcgtctggcggagcgcattctgtacggcccgcagtga
2. Branched-chain amino acid aminotransferase IlvE from E. coli Nissle
SEQ ID NO: 21-IlvE Amino acid sequence:
MSYPEKFEGIAIQS HEDWKNPKKTKYDPKPFYDHDIDIKIEACGVCGSDIHCAAGHW
GNMKMPLVVGHEIVGKVVKLGPKSNSGLKVGQRVGVGAQVFSCLECDRCKNDNEP
YCTKFVTTYSQPYEDGYVSQGGYANYVRVHEHFVVPIPENIPSHLAAPLLCGGLTVY
SPLVRNGCGPGKKVGIVGLGGIGSMGTLISKAMGAETYVISRSSRKREDAMKMGAD
HYIATLEEGDWGEKYFDTFDLIVVCASSLTDIDFNIMPKAMKVGGRIVSISIPEQHEM
LSLKPYGLKAVSISYSALGSIKELNQLLKLVSEKDIKIWVETLPVGEAGVHEAFERME
KGDVRYRFTLVGYDKEFSD
SEQ ID NO: 22-ilvE nucleotide sequence:
atgaccacgaagaaagctgattacataggacaatggggagatggacgctgggaagacgcgaaggtgcatgtgatgtcgcacgcgc
tgcactatggcacctcggtattgaaggcatccgagctacgactcgcacaaaggaccggagtattccgccatcgtgagcatatgcagc
gtctgcatgactccgccaaaatctatcgcacccggatcgcagagcattgatgagctgatggaagatgtcgtgacgtgatccgcaaaa
acaatctcaccagcgcctatatccgtccgctgatcttcgttggtgatgttggcatgggcgtaaacccgccagcgggatactcaaccgac
gtgattatcgccgctacccgtggggagcgtatctgggcgcagaagcgctggagcaggggatcgatgcgatggtacctcctggaacc
gcgcagcaccaaacaccatcccgacggcggcaaaagccggtggtaactacctctcaccctgctggtgggtagcgaagcgcgccgc
cacggttatcaggaaggtatcgcgaggatgtgaatggttacatctctgaaggcgcaggcgaaaacctgatgaagtgaaagacggcg
tgctgacaccccaccgttcacctcatccgcgctgccgggtattacccgtgatgccatcatcaaactggcaaaagagctgggaattgaa
gtgcgtgagcaggtgctgtcgcgcgaatccctgtacctggcggatgaagtgatatgtccggtacggcggcagaaatcacgccagtg
cgcagcgtagacggtattcaggaggcgaaggccgagtggcccggttaccaaacgcattcagcaagccacttcggcctcacactgg
cgaaaccgaagataaatggggctggttagatcaagttaatcaataa
3. L-amino acid deaminase L-AAD from Proteus vulgaris
SEQ ID NO: 23-L-AAD Amino acid sequence:
MAISRRKFIIGGTVVAVAAGAGILTPMLTREGRFVPGTPRHGFVEGTEGALPKQADV
VVVGAGILGIMTAINLVERGLSVVIVEKGNIAGEQSSRFYGQAISYKMPDETFLLHHL
GKHRWREMNAKVGIDTTYRTQGRVEVPLDEEDLVNVRKWIDERSKNVGSDIPFKTR
IIEGAELNQRLRGATTDWKIAGFEEDSGSFDPEVATFVMAEYAKKMGVRIYTQCAAR
GLETQAGVISDVVTEKGAIKTSQVVVAGGVWSRLFMQNLNVDVPTLPAYQSQQLIS
GSPTAPGGNVALPGGIFFREQADGTYATSPRVIVAPVVKESFTYGYKYLPLLALPDFP
VHISLNEQLINSFMQSTHWNLDEVSPFEQFRNMTALPDLPELNASLEKLKAEFPAFKE
SKLIDQWSGAMAIAPDENPIISEVKEYPGLVINTATGWGMTESPVSAELTADLLLGKK
PVLDPKPFSLYRF
SEQ ID NO: 24-L-AAD Codon-optimized nucleotide sequence:
atggccatcagtcgtcgcaaattcattatcggtggaacggtcgtcgccgttgccgccggtgcggggattagaccccgatgctgacgc
gcgaagggcgctagtgccgggcactccacgccacggatcgttgaagggaccgagggggcatacccaaacaagcggacgtggtg
gtcgtaggcgctggaattcaggtattatgacggccattaataggagagcgtgggctgtcagtggtaattgtggagaagggcaatatcg
cgggagaacaaagctctcgcactacggacaggcaattagctataagatgccagatgagacatattgctgcaccatcagggaagcac
cgctggcgtgagatgaatgcgaaagtagggattgatacaacgtaccgtactcaaggacgcgtggaagtaccgcttgacgaggaagat
aggtaaacgtccgcaaatggattgacgaacgttcaaaaaatgaggatctgacattccattaagacccgcattatcgagggggcagaa
ttaaatcagcgtctgcgcggcgccacaacagattggaagatcgctggcttcgaggaggacagcgggtcattcgatcccgaggtagcg
acctagtaatggcagagtacgcgaagaagatgggtgacgtatctatacgcaatgcgcggcccgcggtctggaaacccaggccggt
gtcatttcagatgagtcacggaaaaaggtgcgattaagacctcccaagtggtagtggctggtggggtgtggagtcgtctgacatgcag
aatttaaacgtcgacgtcccaacccacctgcgtatcagtcacagcagttgattagtggacccctaccgcaccgggggggaacgtcgc
attacctggtggaatcacttccgcgaacaggcggacgggacatacgcgacttctcctcgtgtgattgagccccagagtgaaggaga
gcttcacttatggttacaagtacttaccattattagcattgcctgataccctgacacattagcctgaatgaacagttaattaattcgatatgc
aaagtacccactggaacttagacgaagtgtcgccgttcgaacaatttcgcaacatgacagcattacctgacttgcccgaacttaacgcc
agcttagaaaagttaaaggcagagaccctgcatcaaagaatccaagttgatcgaccagtggtctggagcaatggcaattgcgcccga
cgaaaatccaatcataccgaggtgaaggagtacccaggtctggtaattaacacggcgacaggaggggcatgactgaaagtccagtg
tctgctgaacttaccgccgatcactgctggggaagaagccggtgttagatcctaagccattctcactttatcgcattga
4. L-amino acid deaminase L-AAD from Proteus mirabilis
SEQ ID NO: 25-L-AAD Amino acid sequence:
MAISRRKFILGGTVVAVAAGAGVLTPMLTREGRFVPGTPRHGFVEGTGGPLPKQDD
VVVIGAGILGIMTAINLAERGLSVTIVEKGNIAGEQSSRFYGQAISYKMPDETFLLHHL
GKHRWREMNAKVGIDTTYRTQGRVEVPLDEEDLENVRKWIDAKSKDVGSDIPFRTK
MIEGAELKQRLRGATTDWKIAGFEEDSGSFDPEVATFVMAEYAKKMGIKIFTNCAAR
GLETQAGVISDVVTEKGPIKTSRVVVAGGVGSRLFMQNLNVDVPTLPAYQSQQLISA
APNAPGGNVALPGGIFFRDQADGTYATSPRVIVAPVVKESFTYGYKYLPLLALPDFP
VHISLNEQLINSFMQSTHWDLNEESPFEKYRDMTALPDLPELNASLEKLKKEFPAFKE
STLIDQWSGAMAIAPDENPIISDVKEYPGLVINTATGWGMTESPVSAEITADLLLGKK
PVLDAKPFSLYRF
SEQ ID NO: 26-L-AAD Nucleotide sequence:
atggcaataagtagaagaaaatttattcaggtggcacagtggagctgagctgcaggcgctggggattaacacctatgttaacgcgag
aagggcgattgacctggtacgccgagacatggattgagagggaactggcggtccattaccgaaacaagatgatgagagtaattgg
tgcgggtatataggtatcatgaccgcgattaaccagctgagcgtggcttatctgtcacaatcgttgaaaaaggaaatattgccggcgaa
caatcatctcgattctatggtcaagctattagctataaaatgccagatgaaaccacttattacatcacctcgggaagcaccgctggcgtg
agatgaacgctaaagaggtattgataccacttatcgtacacaaggtcgtgtagaagaccatagatgaagaagatttagaaaacgtaag
aaaatggattgatgctaaaagcaaagatgaggctcagacattccatttagaacaaaaatgattgaaggcgctgagttaaaacagcgat
acgtggcgctaccactgattggaaaattgctggatcgaagaagactcaggaagcttcgatcctgaagagcgacttagtgatggcaga
atatgccaaaaaaatgggtatcaaaattacacaaactgtgcagcccgtggatagaaacgcaagctggtgttatactgatgagtaacag
aaaaaggaccaattaaaacctctcgtgagagtcgccggtggtgagggtcacgatatttatgcagaacctaaatgagatgtaccaaca
ttacctgcttatcaatcacagcaattaattagcgcagcaccaaatgcgccaggtggaaacgagattacccggcggaattactacgtga
tcaagcggatggaacgtatgcaacactcctcgtgtcattgagctccggtagtaaaagaatcatttacttacggctataaatatttacctctg
ctggattacctgatacccagtacatatttcgttaaatgagcagttgattaattcattatgcaatcaacacattgggatcttaatgaagagtc
gccatttgaaaaatatcgtgatatgaccgctctgcctgatctgccagaattaaatgcctcactggaaaaactgaaaaaagagttcccagc
atttaaagaatcaacgttaattgatcagtggagtggtgcgatggcgattgcaccagatgaaaacccaattatctctgatgttaaagagtat
ccaggtctagttattaatactgcaacaggaggggaatgactgaaagccctgtatcagcagaaattacagcagatttattattaggcaaaa
aaccagtattagatgccaaaccatttagtctgtatcgtactaa
H. Branched-chain ketoacid decarboxylase sequences
1. KivD from lactococcas lactis strain IFPL730
SEQ ID NO: 27-KivD Amino acid sequence:
MYTVGDYLLDRLHELGIEEIFGVPGDYNLQFLDQIISHKDMKWVGNANELNASYMA
DGYARTKKAAAFLTTFGVGELSAVNGLAGSYAENLPVVEIVGSPTSKVQNEGKFVH
HTLADGDFKHFMKMHEPVTAARTLLTAENATVEIDRVLSALLKERKPVYINLPVDV
AAAKAEKPSLPLKKENSTSNTSDQEILNKIQESLKNAKKPIVITGHEIISFGLEKTVTQF
ISKTKLPITTLNFGKSSVDEALPSFLGIYNGTLSEPNLKEFVESADFILMLGVKLTDSST
GAFTHHLNENKMISLNIDEGKIFNERIQNFDFESLISSLLDLSEIEYKGKYIDKKQEDFV
PSNALLSQDRLWQAVENLTQSNETIVAEQGTSFFGASSIFLKSKSHFIGQPLWGSIGYT
FPAALGSQIADKESRHLLFIGDGSLQLTVQELGLAIREKINPICFIINNDGYTVEREIHG
PNQSYNDIPMWNYSKLPESFGATEDRVVSKIVRTENEFVSVMKEAQADPNRMYWIE
LILAKEGAPKVLKKMGKLFAEQNKS
SEQ ID NO: 28-kivD Nucleotide sequence:
atgtatacagtaggagattacctattagaccgattacacgagttaggaattgaagaaatttaggagtccctggagactataacttacaattt
ttagatcaaattatacccacaaggatatgaaatgggtcggaaatgctaatgaattaaatgcttcatatatggctgatggctatgctcgtact
aaaaaagctgccgcatttcttacaacctaggagtaggtgaattgagtgcagttaatggattagcaggaagttacgccgaaaatttacca
gtagtagaaatagtgggatcacctacatcaaaagttcaaaatgaaggaaaatttgacatcatacgctggctgacggtgatataaacactt
tatgaaaatgcacgaacctgttacagcagctcgaactttactgacagcagaaaatgcaaccgagaaattgaccgagtactactgcact
attaaaagaaagaaaacctgtctatatcaacttaccagttgatgagctgctgcaaaagcagagaaaccctcactccattgaaaaagga
aaactcaacttcaaatacaagtgaccaagaaattagaacaaaattcaagaaagcttgaaaaatgccaaaaaaccaatcgtgattacag
gacatgaaataattagattggcttagaaaaaacagtcactcaatttatttcaaagacaaaactacctattacgacattaaactaggtaaaa
gacagttgatgaagccctccatcattataggaatctataatggtacactctcagagcctaatcttaaagaattcgtggaatcagccgact
tcatcttgatgatggagttaaactcacagactatcaacaggagcatcactcatcatttaaatgaaaataaaatgatttcactgaatatag
atgaaggaaaaatatttaacgaaagaatccaaaattagattagaatccctcatctcctctctcttagacctaagcgaaatagaatacaaa
ggaaaatatatcgataaaaagcaagaagactagaccatcaaatgcgcattatcacaagaccgcctatggcaagcagttgaaaaccta
actcaaagcaatgaaacaatcgagctgaacaagggacatcattctaggcgcttcatcaattacttaaaatcaaagagtcatatattggtc
aacccttatggggatcaattggatatacattcccagcagcattaggaagccaaattgcagataaagaaagcagacaccattatttattgg
tgatggacacttcaacttacagtgcaagaattaggattagcaatcagagaaaaaattaatccaatttgattattatcaataatgatggttat
acagtcgaaagagaaattcatggaccaaatcaaagctacaatgatattccaatgtggaattactcaaaattaccagaatcgtaggagca
acagaagatcgagtagtctcaaaaatcgttagaactgaaaatgaatttgtgtctgtcatgaaagaagctcaagcagatccaaatagaatg
tactggattgagttaattaggcaaaagaaggtgcaccaaaagtactgaaaaaaatgggcaaactatttgctgaacaaaataaatcataa
SEQ ID NO: 29-kivD Codon-optimized sequence:
atgtatacagtaggagattacttattggaccggagcacgaacttggaattgaggaaatattggagaccgggtgactacaacctgcagtt
ccttgaccaaatcatctcccataaggacatgaaatgggtcggcaatgccaatgagctgaacgcatcatatatggcagacgggtatgctc
ggaccaaaaaggctgcagcattccttaccacgtttggcgtgggggaattaagtgctgtaaatggactggcaggatcctatgcggagaa
ataccggtagtcgaaattgaggctcgcctacgtccaaggtgcagaatgaggggaaattcgtccatcacacacttgcagacggtgatat
aagcactttatgaagatgcatgagccggtaacggctgcgcggacgcacttactgcggaaaacgcaacagtagagattgatcgcgact
gagcgcactgcttaaggaacggaagcccgtctatattaacttaccggtagacgtggccgcagccaaagccgaaaaaccaagcctgc
ctcttaagaaggagaattccacgtccaacaccagtgaccaagagattagaacaaaattcaagagtattgaagaacgcgaagaagcc
catcgtaattacaggacatgagattatctcgtaggcctggagaaaacggttacacagatataccaaaacgaagttacctataacgacgt
taaactaggaaagagctctgtggatgaggcacttcctagtacttaggaatctataatgggaccattcagagccaaacttaaaggaattc
gagaaagtgcggatatatcttaatgcttggggttaaattgactgattccagcaccggagatttacgcaccatttaaacgagaacaaaat
gatctattgaatatcgacgaaggcaaaattataatgaaagaattcagaactagattagaatcccttattagttcactatagatttaagtga
aatagagtataagggaaagtatatagacaagaagcaagaggatttcgaccgtctaatgctcattaagtcaagacagactaggcaggc
ggagagaaccttacacaatccaatgaaacgatagtcgccgaacaagggaccagtacttcggcgcttatccatattcctgaagtctaa
gtctcatttcattggacagcccctgtgggggtctataggatatacgtacccgcagctcaggaagccagatcgccgataaggagagca
gacacctgagttcatcggggacggctcgagcagctgactgacaggaactggggaggcgatcagagagaagattaatcccatttgct
ttatcataaataatgatggttataccgtagaacgtgagattcatggacctaatcagagctataatgacattcctatgtggaactattcaaaat
tgccagagagattggtgcaactgaggatcgcgagttagtaaaatagtccgcacggagaacgagtagtcagcgtaatgaaggaggc
ccaagcggaccctaatcggatgtactggatcgaacttattctggctaaagaaggagcacctaaagattaaagaagatgggaaaactat
tgctgaacaaaataaatcataa
2. KdcA amino acid sequence from lactococcas lactis strain B1157
SEQ ID NO: 30-KdcA Amino acid sequence:
MYTVGDYLLDRLHELGIEEIFGVPGDYNLQFLDQIISREDMKWIGNANELNASYMAD
GYARTKKAAAFLTTFGVGELSAINGLAGSYAENLPVVEIVGSPTSKVQNDGKFVHHT
LADGDFKHFMKMHEPVTAARTLLTAENATYEIDRVLSQLLKERKPVYINLPVDVAA
AKAEKPALSLEKESSTTNTTEQVILSKIEESLKNAQKPVVIAGHEVISFGLEKTVTQFV
SETKLPITTLNFGKSAVDESLPSFLGIYNGKLSEISLKNFVESADFILMLGVKLTDSSTG
AFTHHLDENKMISLNIDEGIIFNKVVEDFDFRAVVSSLSELKGIEYEGQYIDKQYEEFIP
SSAPLSQDRLWQAVESLTQSNETIVAEQGTSFFGASTIFLKSNSRFIGQPLWGSIGYTF
PAALGSQIADKESRHLLFIGDGSLQLTVQELGLSIREKLNPICFIINNDGYTVEREIHGP
TQSYNDIPMWNYSKLPETFGATEDRVVSKIVRTENEFVSVMKEAQADVNRMYWIEL
VLEKEDAPKLLKKMGKLFAEQNKS
SEQ ID NO: 31-kdcA Nucleotide sequence:
atgtatacagtaggagattacctattagaccgattacacgagagggaattgaagaaatttaggagacctggtgactataacttacaatat
tagatcaaattatttcacgcgaagatatgaaatggattggaaatgctaatgaattaaatgatcttatatggctgatggttatgctcgtactaa
aaaagctgccgcatactcaccacataggagtcggcgaattgagtgcgatcaatggactggcaggaagttatgccgaaaatttaccagt
agtagaaattgaggacaccaacttcaaaagtacaaaatgacggaaaatagtccatcatacactagcagatggtgatataaacactttat
gaagatgcatgaacctgttacagcagcgcggactttactgacagcagaaaatgccacatatgaaattgaccgagtactactcaattact
aaaagaaagaaaaccagtctatattaacttaccagtcgatgagctgcagcaaaagcagagaagcctgcattatattagaaaaagaaa
gctctacaacaaatacaactgaacaagtgattagagtaagattgaagaaagatgaaaaatgcccaaaaaccagtagtgattgcagga
cacgaagtaattagattggatagaaaaaacggtaactcagatgatcagaaacaaaactaccgattacgacactaaattaggtaaaagt
gctgagatgaatcatgccctcattataggaatatataacgggaaactacagaaatcagtcttaaaaattagtggagtccgcagactttat
cctaatgcaggagtgaagcttacggactcctcaacaggtgcattcacacatcatttagatgaaaataaaatgatttcactaaacatagatg
aaggaataattacaataaagtggtagaagattagatatagagcagtggatatattatcagaattaaaaggaatagaatatgaaggac
aatatattgataagcaatatgaagaatttattccatcaagtgctcccttatcacaagaccgtctatggcaggcagttgaaagatgactcaa
agcaatgaaacaatcgagctgaacaaggaacctcattattggagcttcaacaattacttaaaatcaaatagtcgattattggacaacctt
tatggggactattggatatacttaccagcggattaggaagccaaattgcggataaagagagcagacaccattatttattggtgatggtt
cacttcaacttaccgtacaagaattaggactatcaatcagagaaaaactcaatccaatttgattatcataaataatgatggttatacagaga
aagagaaatccacggacctactcaaagttataacgacattccaatgtggaattactcgaaattaccagaaacataggagcaacagaag
atcgtgtagtatcaaaaattgttagaacagagaatgaatagtgtctgtcatgaaagaagcccaagcagatgtcaatagaatgtattggat
agaactagattggaaaaagaagatgcgccaaaattactgaaaaaaatgggcaaactatttgctgaacaaaataaatcataa
SEQ ID NO: 32-kdcA Codon-optimized kdcA sequence:
atgtatacagtaggagattaccattagatcgtagcacgaattgggcattgaggaaatattggcgtccctggcgactacaatttacaattct
tagatcagattatttcacgtgaggatatgaagtggattgggaatgccaatgagctgaacgcgagctatatggcggacggttacgctcgt
acaaaaaaggcagcagcgtacttactacttaggcgtaggcgaattgtcggccatcaacgggcttgcgggacgtatgcggaaaactta
ccggagtcgagattgtcggacccctacttcgaaggtgcagaatgatggcaaattcgttcatcacaccaggcagacggcgactttaaa
catttcatgaaaatgcacgaacctgtgactgccgcccgcacacactgacagctgaaaacgcgacatacgaaattgatcgcgtgattc
gcagttgagaaagagcgtaaacccgtatatatcaatctgccggtggatgtagcggctgcaaaagccgaaaaaccggcgctgtcactg
gaaaaagaatcgtctacgactaatacaacggaacaagtaatcctgtcaaaaatcgaagagagcttgaaaaacgcccagaagcctgtc
gtgattgccgggcacgaggtcattagattgggttagaaaagactgttacccagttcgtgagtgagacgaagagcccatcaccaccctt
aactaggcaagtctgcggtagacgagagcttaccgtattataggtatctacaatgggaaactacagaaatttcactgaaaaacttcgtg
gagtcggcagactttatataatgagggtgttaaattaactgatagcagcactggcgcgttcacgcatcacttggatgagaataaaatgat
ctcgcttaacatcgacgaaggtatcattataataaagagtagaggacttcgactacgtgctgagtatcgagccatccgaattaaagggt
atcgagtacgaaggtcagtacattgacaagcaatacgaggaatttatcccctccagcgcgcctcttagccaagaccgcctaggcagg
ccgtagagagtcttacacaaagtaatgaaactattgagcagaacagggtacaagatctaggcgcctcgacgattacttaaaatcgaa
cagtcgattatcgggcaacctctagggggtcgattgggtacaccatcctgcggccttaggctctcaaattgcggacaaagaatctcgc
catttattattcatcggcgacggctcgttacagcttacagtgcaagagagggattatcgattcgcgagaagctgaatccgatttgattatc
attaacaacgacgggtacacagtcgaacgcgaaatccatggcccgacacaatcatataatgacatccctatgtggaattattctaagctt
ccagagacattcggcgcaactgaagaccgcgtcgtgtcaaaaattgtccgcactgagaatgaattcgtgtcagtgatgaaggaagctc
aggccgatgtcaaccgcatgtactggattgaattagattggagaaagaggatgcccccaaattacttaagaagatggggaaactatttg
ctgaacaaaataaatcataa
3. THI3/KID1 from Saccharomyces cerevisiae
SEQ ID NO: 33-THI3/KID1 Amino acid sequence:
MNSSYTQRYALPKCIAISDYLFHRLNQLNIHTIFGLSGEFSMPLLDKLYNIPNLRWAG
NSNELNAAYAADGYSRLKGLGCLITTFGVGELSAINGVAGSYAEHVGILHIVGMPPT
SAQTKQLLLHHTLGNGDFTVFHRIASDVACYTTLIIDSELCADEVDKCIKKAWIEQRP
VYMGMPVNQVNLPIESARLNTPLDLQLHKNDPDVEKEVISRILSFIYKSQNPAIIVDA
CTSRQNLIEETKELCNRLKFPVFVTPMGKGTVNETDPQFGGVFTGSISAPEVREVVDF
ADFIIVIGCMLSEFSTSTFHFQYKTKNCALLYSTSVKLKNATYPDLSIKLLLQKILANL
DESKLSYQPSEQPSMMVPRPYPAGNVLLRQEWVWNEISHWFQPGDIIITETGASAFG
VNQTRFPVNTLGISQALWGSVGYTMGACLGAEFAVQEINKDKFPATKHRVILFMGD
GAFQLTVQELSTIVKWGLTPYIFVMNNQGYSVDRFLHHRSDASYYDIQPWNYLGLL
RVFGCTNYETKKIITVGEFRSMISDPNFATNDKIRMIEIMLPPRDVPQALLDRWVVEK
EQSKQVQEENENSSAVNTPTPEFQPLLKKNQVGY
SEQ ID NO: 34-THI3/KID1 Nucleotide sequence:
atgaattctagctatacacagagatatgcactgccgaagtgtatagcaatatcagattatcattccatcggctcaaccagctgaacataca
taccatataggactctccggagaatttagcatgccgagctggataaactatacaacattccgaacttacgatgggccggtaattctaatg
agttaaatgctgcctacgcagcagatggatactcacgactaaaaggcagggatgtctcataacaacctaggtgtaggcgaattatcgg
caatcaatggcgtggccggatcttacgctgaacatgtaggaatacttcacatagtgggtatgccgccaacaagtgcacaaacgaaaca
actactactgcatcatactctgggcaatggtgatttcacggtatttcatagaatagccagtgatgtagcatgctatacaacattgattattga
ctctgaattatgtgccgacgaagtcgataagtgcatcaaaaaggcaggatagaacagaggccagtatacatgggcatgcctgtcaac
caggtaaatctcccgattgaatcagcaaggcttaatacacctctggatttacaattgcataaaaacgacccagacgtagagaaagaagtt
atactcgaatattgagattatatacaaaagccagaatccggcaatcatcgtagatgcatgtactagtcgacagaatttaatcgaggagac
taaagagctagtaataggcttaaataccagtattgttacacctatgggtaagggtacagtaaacgaaacagacccgcaatagggggc
gtattcacgggctcgatatcagccccagaagtaagagaagtagttgattagccgatatatcatcgtcattggagcatgctctccgaattc
agcacgtcaactaccacttccaatataaaactaagaattgtgcgctactatattctacatctgtgaaattgaaaaatgccacatatcctgac
ttgagcattaaattactactacagaaaatattagcaaatcttgatgaatctaaactgtcttaccaaccaagcgaacaacccagtatgatggt
tccaagaccttacccagcaggaaatgtcctcttgagacaagaatgggtctggaatgaaatatcccattggaccaaccaggtgacataat
cataacagaaactggtgatctgcataggagttaaccagaccagataccggtaaatacactaggtatttcgcaagctctaggggatctg
tcggatatacaatgggggcgtgtcaggggcagaatttgctgacaagagataaacaaggataaattccccgcaactaaacatagagtta
actgatatgggtgacggtgctaccaattgacagttcaagaattatccacaattgttaagtggggattgacaccttatatattgtgatgaat
aaccaaggttactctgtggacaggtattgcatcacaggtcagatgctagttattacgatatccaaccaggaactacttgggattattgcg
agtataggagcacgaactacgaaacgaaaaaaattattactgaggagaattcagatccatgatcagtgacccaaactagcgaccaat
gacaaaattcggatgatagagattatgctaccaccaagggatgttccacaggctctgcttgacaggtgggtggtagaaaaagaacaga
gcaaacaagtgcaagaggagaacgaaaattctagcgcagtaaatacgccaactccagaattccaaccacttctaaaaaaaaatcaagt
tggatactga
4. ARO10 from Saccharomyces cerevisiae
SEQ ID N : 35-ARON Amino acid sequence:
MAPVTIEKFVNQEERHLVSNRSATIPFGEYIFKRLLSIDTKSVFGVPGDFNLSLLEYLY
SPSVESAGLRWVGTCNELNAAYAADGYSRYSNKIGCLITTYGVGELSALNGIAGSFA
ENVKVLHIVGVAKSIDSRSSNFSDRNLHHLVPQLHDSNFKGPNHKVYHDMVKDRVA
CSVAYLEDIETACDQVDNVIRDIYKYSKPGYIFVPADFADMSVTCDNINNVPRISQQ
DCIVYPSENQLSDIINKITSWIYSSKTPAILGDVLTDRYGVSNELNKLICKTGIWNFSTV
MGKSVIDESNPTYMGQYNGKEGLKQVYEHFELCDLVLHFGVDINEINNGHYTFTYK
PNAKIIQFHPNYIRLVDTRQGNEQMFKGINFAPILKELYKRIDVSKLSLQYDSNVTQY
TNETMRLEDPTNGQSSIITQVHLQKTMPKFLNPGDVVVCETGSFQFSVRDFAFPSQLK
YISQGFFLSIGMALPAALGVGIAMQDHSNAIIINGGNVKEDYKPRLILFEGDGAAQMT
IQELSTILKCNIPLEVIIWNNNGYTIERAIMGPTRSYNDVMSWKWTKLFEAFGDFDGK
YINSTLIQCPSKLALKLEELKNSNKRSGIELLEVKLGELDFPEQLKCMVEAAALKRN
KK
SEQ ID NO:36-ARO10 Nucleotide sequence:
atggcacctgttacaattgaaaagttcgtaaatcaagaagaacgacaccttgatccaaccgatcagcaacaattccgtttggtgaataca
tatttaaaagattgagtecatcgatacgaaatcagattcggtattcctagtgacttcaacttatctctattagaatatctctattcacctagtgt
tgaatcagctggcctaagatgggtcggcacgtgtaatgaactgaacgccgcttatgcggccgacggatattcccgttactctaataaga
ttggctatttaataaccacgtatggcgaggtgaattaagcgccttaaacggtataaccgccgttcgctaaaaatgtcaaagattgcac
attgaggtgtggccaagtccatagattcgcccaagtaactttagtgatcggaacctacatcatttggtcccacagetacatgattcaaat
ataaagggccaaatcataaaatatatcatgatatggtaaaagataaagtcgcttgctcggtagcctacttggaggatattgaaactgcat
gtgaccaagtcgataatgttatccgcgatatttacaagtattctaaacctggttataatttgacctgcagattttgcggatatgtctgttacat
gtgataataggttaatgaccacgtatatctcaacaaaattgtatagtataccatctgaaaaccaattgtctgacataatcaacaagattact
agttggatatattccagtaaaacacctgcgatccaggagacgtactgactgataggtatggtgtgagtaactattgaacaagcttatctg
caaaactgggatttggaattatccactattatgggaaaatctgtaattgatgagtcaaacccaacttatatgggtcaatataatggtaaaaa
aggtttaaaacaagtctatgaacattagaactcgcgacttggtcttgcattaggatttcgacatcaatttaaattaataatgggcattatact
tttacttataaaccaaatgctaaaatcattcaatttcatccgaattatattcgccttgtggacactaggcagggcaatgagcaaatgttcaaa
ggaateaattagcccctattttaaaagaactatacaagcgcattgacgtttctaaactttcatgcaatatgattcaaatgtaactcaatatac
gaacaaaacaatgcggttagaagatcctaccaatggacaatcaagcattattacacaaattcacttacaaaagacgatgcctaaattttta
aaccctggtgatgagtcgtagtgaaacagttctctatcaattctagttcgtgatttcgcgatccttcgcaattaaaatatatatcgcaagg
atattcctaccattggcatggcccacctgccgccctaggtgttaaaattgccatgcaagaccactcaaacgctcacatcaatggtggca
acgtaaaagaggactataagecaagattaattttgatgaaggtgacttcgcagcacattatgacaatccaagaactttgcaccattcttt
aagtgcaatattccactagaaattatcataggaacaataacggctacactattaaaagagccatcatgggccctaccaggtcgtataac
ttacgttatgtcattttaaatggaccaaactatttgaagcattcggagacttcgacggaaagtatactaatagcactctcattcaatgtccctc
taaattageactgaaattggaggagcttaagaattcaaacaaaagaagcgggatagaactatagaagtcaaattaggcgaattggant
cecettaacagctaaagtgcatggttgaagcageggcacttaaaattaaataaaaaatag
III. Alcohol dehydrogenase sequences
1. Adh2 from Saccharomyces cerevisae
SEQ ID NO: 37-Adh2 Amino acid sequence:
MSIPETQKAIIFYESNGKLEHKDIPVPKPKPNELLINVKYSGVCHTDLHAWHGDWPLP
TKLPLVGGHEGAGVVVGMGENVKGWKIGDYAGIKWLNGSCMACEYCELGNESNC
PHADLSGYTHDGSFQEYATADAVQAAHIPQGTDLAEVAPILCAGITVYKALKSANLR
AGHWAAISGAAGGLGSLAVQYAKAMGYRVLGIDGGPGKEELFTSLGGEVFIDFTKE
KDIVSAVVKATNGGAHMINVSVSEAMEASTRYCRANGTVVLVGLPAGAKCSSDVF
NHVVKSISIVGSYVGNRADTREALDFFARGLVKSPIKVVGLSSLPEIYEKMEKGQIAG
RYVVDTSK
SEQ ID NO: 38-adh2 Nucleotide sequence:
atgtctattccagaaactcaaaaagccattatcactacgaatccaacggcaagaggagcataaggatatcccagaccaaagccaaag
cccaacgaattgttaatcaacgtcaagtactctggtgtctgccacaccgatttgcacgcaggcatggtgactggccattgccaactaagt
taccattagaggtggtcacgaaggtgccggtgtcgagtcggcatgggtgaaaacgttaagggctggaagatcggtgactacgccgg
tatcaaatggagaacggacttgtatggcctgtgaatactgtgaattgggtaacgaatccaactgtcctcacgctgacttgtctggttacac
ccacgacggactaccaagaatacgctaccgctgacgctgacaagccgctcacattcctcaaggtactgacttggctgaagtcgcgcc
aatcagtgtgctggtatcaccgtatacaaggattgaagtctgccaacttgagagcaggccactgggcggccatactggtgctgctggt
ggtctaggactaggctgacaatatgctaaggcgatgggttacagagtcttaggtattgatggtggtccaggaaaggaagaattgatac
ctcgctcggtggtgaagtattcatcgacttcaccaaagagaaggacattgttagcgcagtcgttaaggctaccaacggcggtgcccac
ggtatcatcaatgatccgtaccgaagccgctatcgaagatctaccagatactgtagggcgaacggtactgagtcaggaggatgcc
agccggtgcaaagtgctcctctgatgtcttcaaccacgagtcaagtctatctccattgtcggctcttacgtggggaacagagctgatacc
agagaagccttagatttctttgccagaggtctagtcaagtctccaataaaggtagttggcttatccagtttaccagaaatttacgaaaagat
ggagaagggccaaattgctggtagatacgttgagacacttctaaataa
SEQ ID NO: 39-adh2 Codon-optimized sequence:
atgtctattccagaaacgcagaaagccatcatatatatgaatcgaacggaaaacttgagcacaaggacatccccgtcccgaagccaaa
acctaatgagagcttatcaacgttaagtattcgggcgtatgccacacagacttgcacgcatggcacggggattggcccttaccgactaa
gagccgttagtgggcggacatgagggggcgggagtcgtagtgggaatgggagagaacgtgaagggaggaagattggagattatg
ctgggattaagtggagaatgggagctgcatggcctgcgaatattgtgaacttggaaatgagagcaattgcccacatgctgacttgtccg
gttacacacatgacggttcattccaggaatatgctacggctgatgcagtccaagcagcgcatatcccgcaagggacggacttagcaga
agtagcgcccattattgcgctgggatcaccgtatataaagcgttaaagagcgcaaatttacgggccggacattgggcggcgatcagc
ggggccgcaggggggctgggcagcaggccgtccagtacgctaaagctatgggttatcgggattgggcattgacggaggaccggg
aaaggaggaattattcacgtccagggaggagaggtattcattgactttaccaaggaaaaagatatcgtctctgctgtagtaaaggctac
caatggcggtgcccacggaatcataaatgatcagtactgaagcggcgatcgaagcgtccactagatattgccgtgcaaatgggacag
tcgtacttgtaggacttccggctggcgccaaatgcagctccgatgtatttaatcatgtcgtgaagtcaatctctatcgaggacatatgtag
gaaaccgcgccgatactcgtgaggctcttgacttattgccagaggcctggttaagtcccccataaaagttgaggcttatccagcttacc
cgaaatatacgagaagatggagaagggccagatcgcggggagatacgttgagacacttctaaataa
2. Adh6 from Saccharomyces cerevisae
SEQ ID NO:40-Adh6 Amino acid sequence:
MSYPEKFEGIAIQSHEDWKNPKKTKYDPKPFYDHDIDIKIEACGVCGSDIHCAAGHW
GNMKMPLVVGHEIVGKVVKLGPKSNSGLKVGQRVGVGAQVFSCLECDRCKNDNEP
YCTKFVTTYSQPYEDGYVSQGGYANYVRVHEHFVVPIPENIPSHLAAPLLCGGLTVY
SPLVRNGCGPGKKVGIVGLGGIGSMGTLISKAMGAETYVISRSSRKREDAMKMGAD
HYIATLEEGDWGEKYFDTFDLIVVCASSLTDIDFNIMPKAMKVGGRIVSISIPEQHEM
LSLKPYGLKAVSISYSALGSIKELNQLLKLVSEKDIKIWVETLPVGEAGVHEAFERME
KGDVRYRFTLVGYDKEFSD
SEQ ID NO:41-adh6 Codon-optimized sequence:
atgtcataccctgaaaaattcgagggtatcgccattcagagtcacgaagattggaagaatcccaagaagaccaaatacgaccccaagc
cgactatgaccatgatatcgacatcaaaatcgaggcatgtggtgtgtgtggcagtgatattcattgcgcagcgggccattgggggaac
atgaagatgcctctggtagtaggacatgagatcgaggaaaggagtgaaattgggtccgaaaagtaactccggtcttaaagtaggtca
gcgtgaggggtcggggcgcaagattcagagcctggagtgtgatcgagtaagaacgataacgagccgtactgcacaaagtagtaa
cgacgtattcacagccatatgaggatgggtatgatctcaagggggctatgcaaactacgtccgcgtacatgaacactagtggtgcctat
tcctgagaacattccgtctcacttggccgctcattgagtgcggaggtcttaccgtctactcgccattggacgcaatgggtgcggtccg
ggcaaaaaggtagggatcgaggccaggtggtatcggatctatgggaacgttaatcagtaaggcgatgggagctgagacctacgttat
acccgttcatcacgtaagcgtgaggatgcgatgaagatgggtgcagatcactacatcgcaacgttagaagagggagattggggcga
aaaatattagacacattgacttgattgtggtagtgcatcgtcacttacagacattgactttaatattatgccaaaggcaatgaaggtaggt
gggcgtattgtgtccatactatcccggaacaacacgagatgctactctgaaaccctacggacttaaagctgtgtccatttcgtacagtgc
ccaggatctatcaaggaactgaatcagctgctgaagcttgatcggagaaagacattaagatagggtggagacattgccagtggggg
aggccggcgttcacgaggcgatgaacgcatggagaagggagatgacgctatcgcttcacgctggaggttatgataaagaattcagt
gattag
3. Adh1 from Saccharomyces cerevisae
SEQ ID NO: 42-Adh1 Amino acid sequence:
MSIPETQKGVIFYESHGKLEYKDIPVPKPKANELLINVKYSGVCHTDLHAWHGDWPL
PVKLPLVGGHEGAGVVVGMGENVKGWKIGDYAGIKWLNGSCMACEYCELGNESN
CPHADLSGYTHDGSFQQYATADAVQAAHIPQGTDLAQVAPILCAGITVYKALKSAN
LMAGHWVAISGAAGGLGSLAVQYAKAMGYRVLGIDGGEGKEELFRSIGGEVFIDFT
KEKDIVGAVLKATDGGAHGVINVSVSEAAIEASTRYVRANGTTVLVGMPAGAKCCS
DVFNQVVKSISIVGSYVGNRADTREALDFFARGLVKSPIKVVGLSTLPEIYEKMEKGQ
IVGRYVVDTSK
SEQ ID NO:43-adh1 Nucleotide sequence:
atgtctatcccagaaactcaaaaaggtgttatcactacgaatcccacggtaagaggaatacaaagatattccagaccaaagccaaag
gccaacgaattgagatcaacgttaaatactctggtgtctgtcacactgacttgcacgcaggcacggtgactggccattgccagttaagc
taccattagtcggtggtcacgaaggtgccggtgtcgagtcggcatgggtgaaaacgttaagggctggaagatcggtgactacgccgg
tatcaaatggagaacggacttgtatggcctgtgaatactgtgaattgggtaacgaatccaactgtcctcacgctgacttgtctggttacac
ccacgacggactaccaacaatacgctaccgctgacgctgacaagccgctcacattcctcaaggtaccgacttggcccaagtcgccc
ccatcagtgtgctggtatcaccgtctacaaggctttgaagtctgctaacttgatggccggtcactgggagctatctccggtgctgctggt
ggtctaggactaggctgacaatacgccaaggctatgggttacagagtcagggtattgacggtggtgaaggtaaggaagaattattca
gatccatcggtggtgaagtatcattgacttcactaaggaaaaggacattgtcggtgctgactaaaggccactgacggtggtgctcacg
gtgtcatcaacgtttccgtttccgaagccgctattgaagcttctaccagatacgttagagctaacggtaccaccgttttggtcggtatgcca
gctggtgccaagtgagactgatgtatcaaccaagtcgtcaagtccatctctattgaggacttacgtcggtaacagagctgacaccag
agaagctttggacttcttcgccagaggtttggtcaagtctccaatcaaggttgtcggcttgtctaccttgccagaaatttacgaaaagatg
gaaaagggtcaaatcgaggtagatacgttgagacacactaaataa
4. Adh3 from Saccharomyces cerevisae
SEQ ID NO: 44-Adh3 Amino acid sequence:
MLRTSTLFTRRVQPSLFSRNILRLQSTAAIPKTQKGVIFYENKGKLHYKDIPVPEPKPN
EILINVKYSGVCHTDLHAWHGDWPLPVKLPLVGGHEGAGVVVKLGSNVKGWKVG
DLAGIKWLNGSCMTCEFCESGHESNCPDADLSGYTHDGSFQQFATADAIQAAKIQQ
GTDLAEVAPILCAGVTVYKALKEADLKAGDWVAISGAAGGLGSLAVQYATAMGYR
VLGIDAGEEKEKLFKKLGGEVFIDFTKTKNMVSDIQEATKGGPHGVINVSVSEAAISL
STEYVRPCGTVVLVGLPANAYVKSEVFSHVVKSINIKGSYVGNRADTREALDFFSRG
LIKSPIKIVGLSELPKVYDLMEKGKILGRYVVDTSK
SEQ ID NO: 45-adh3 Nucleotide sequence:
atgagagaacgtcaacattgacaccaggcgtgtccaaccaagcctattactagaaacattcttagattgcaatccacagctgcaatccc
taagactcaaaaaggtgtcatcttttatgagaataaggggaagctgcattacaaagatatccctgtccccgagcctaagccaaatgaaat
ataatcaacgttaaatattctggtgtatgtcacaccgatttacatgatggcacggcgattggccattacctgttaaactaccattagtaggt
ggtcatgaaggtgctggtgtagagtcaaactaggaccaatgtcaagggctggaaagtcggtgatttagcaggtatcaaatggctgaac
ggacttgtatgacatgcgaattctgtgaatcaggtcatgaatcaaattgtccagatgctgatttatctggttacactcatgatggactacca
acaatttgcgaccgctgatgctattcaagccgccaaaattcaacagggtaccgacttggccgaagtagccccaatattatgtgctggtgt
tactgtatataaagcactaaaagaggcagacttgaaagctggtgactgggttgccatctctggtgctgcaggtggcttgggttccttggc
cgttcaatatgcaactgcgatgggttacagagactaggtattgatgcaggtgaggaaaaggaaaaactatcaagaaattggggggtg
aagtattcatcgactttactaaaacaaagaatatggtactgacattcaagaagctaccaaaggtggccctcatggtgtcattaacgtacc
gtactgaagccgctatactctatctacggaatatgttagaccatgtggtaccgtcgattggaggatgcccgctaacgcctacgttaaat
cagaggtattctctcatgtggtgaagtccatcaatatcaagggacttatgaggtaacagagctgatacgagagaagccttagacttatt
agcagaggatgatcaaatcaccaatcaaaattgaggattatctgaattaccaaaggatatgacttgatggaaaagggcaagattaggg
tagatacgtcgtcgatactagtaaataa
5. Adh4 from Saccharomyces cerevisae
SEQ ID NO: 46-Adh4 Amino acid sequence:
MSSVTGFYIPPISFFGEGALEETADYIKNKDYKKALIVTDPGIAAIGLSGRVQKMLEER
DLNVAIYDKTQPNPNIANVTAGLKVLKEQNSEIVVSIGGGSAHDNAKAIALLATNGG
EIGDYEGVNQSKKAALPLFAINTTAGTASEMTRFTIISNEEKKIKMAIIDNNVTPAVAV
NDPSTMFGLPPALTAATGLDALTHCIEAYVSTASNPITDACALKGIDLINESLVAAYK
DGKDKKARTDMCYAEYLAGMAFNNASLGYVHALAHQLGGFYHLPHGVCNAVLLP
HVQEANMQCPKAKKRLGEIALHFGASQEDPEETIKALHVLNRTMNIPRNLKELGVKT
EDFEILAEHAMHDACHLTNPVQFTKEQVVAIIKKAYEY
SEQ ID NO:47-adh4 Nucleotide sequence:
atgtcaccgttactgggattacattccaccaatctctactaggtgaaggtgattagaagaaaccgctgattacatcaaaaacaaggatt
acaaaaaggattgatcgttactgatcctggtattgcagctattggtctctccggtagagtccaaaagatgaggaagaacgtgacttaaa
cgagctatctatgacaaaactcaaccaaacccaaatattgccaatgtcacagctggatgaaggattgaaggaacaaaactctgaaatt
gagtaccattggtggtggactgctcacgacaatgctaaggccattgattattggctactaacggtggggaaatcggagactatgaag
gtgtcaatcaatctaagaaggctgattaccactatttgccatcaacactactgctggtactgcaccgaaatgaccagattcactattatct
ctaatgaagaaaagaaaatcaagatggctatcattgacaacaacgtcactccagctgagctgtcaacgatccatctaccatgtaggat
gccacctgctttgactgctgctactggtctagatgctttgactcactgtatcgaagcttatgtttccaccgcctctaacccaatcaccgatgc
ctgtgattgaagggtattgatttgatcaatgaaagcttagtcgctgcatacaaagacggtaaagacaagaaggccagaactgacatgt
gttacgctgaatacttggcaggtatggcatcaacaatgatctctaggttatgacatgccatgctcatcaacttggtggatctaccacttg
cctcatggtgatgtaacgctgtcttgagcctcatgacaagaggccaacatgcaatgtccaaaggccaagaagagattaggtgaaattg
attgcatttcggtgatctcaagaagatccagaagaaaccatcaaggctagcacgattaaacagaaccatgaacattccaagaaactt
gaaagaattaggtgttaaaaccgaagattagaaattaggctgaacacgccatgcatgatgcctgccatttgactaacccagttcaattca
ccaaagaacaagtggttgccattatcaagaaagcctatgaatattaa
6. Adh5 from Saccharomyces cerevisae
SEQ ID NO: 48-Adh5 Amino acid sequence:
MPSQVIPEKQKAIVFYETDGKLEYKDVTVPEPKPNEILVHVKYSGVCHSDLHAWHG
DWPFQLKFPLIGGHEGAGVVVKLGSNVKGWKVGDFAGIKWLNGTCMSCEYCEVGN
ESQCPYLDGTGFTHDGTFQEYATADAVQAAHIPPNVNLAEVAPILCAGITVYKALKR
ANVIPGQWVTISGACGGLGSLAIQYALAMGYRVIGIDGGNAKRKLFEQLGGEIFIDFT
EEKDIVGAIIKATNGGSHGVINVSVSEAAIEASTRYCRPNGTVVLVGMPAHAYCNSD
VFNQVVKSISIVGSCVGNRADTREALDFFARGLIKSPIHLAGLSDVPEIFAKMEKGEIV
GRYVVETSK
SEQ ID NO: 49-adh5 Nucleotide sequence:
atgccacgcaagtcattcctgaaaaacaaaaggctattgtcattatgagacagatggaaaattggaatataaagacgtcacagaccgg
aacctaagcctaacgaaatatagtccacgttaaatattctggtgatgtcatagtgacttgcacgcgtggcacggtgattggccatttcaat
tgaaataccattaatcggtggtcacgaaggtgctggtgagagttaagagggatctaacgttaagggctggaaagtcggtgattagca
ggtataaaatggagaatgggacttgcatgtcctgtgaatattgtgaagtaggtaatgaatctcaatgtccttataggatggtactggcttc
acacatgatggtactatcaagaatacgcaactgccgatgccgttcaagctgcccatattccaccaaacgtcaatcagctgaagagccc
caatcagtgtgcaggtatcactgatataaggcgttgaaaagagccaatgtgataccaggccaatgggtcactatatccggtgcatgcg
gtggcagggactctggcaatccaatacgccatgctatgggttacagggtcattggtatcgatggtggtaatgccaagcgaaagttattt
gaacaattaggcggagaaatattcatcgatttcacggaagaaaaagacattgaggtgctataataaaggccactaatggcggactcat
ggagttattaatgtgtctgatctgaagcagctatcgaggcactacgaggtattgtaggcccaatggtactgtcgtcctggaggtatgcc
agctcatgcttactgcaattccgatgattcaatcaagagtaaaatcaatctccatcgaggatcagtgaggaaatagagctgatacaag
ggaggcatagatacttcgccagaggatgatcaaatctccgatccacttagctggcctatcggatgacctgaaatattgcaaagatgga
gaagggtgaaattgaggtagatatgagttgagacactaaatga
7. Adh7 from Saccharomyces cerevisae
SEQ ID NO: 50-Adh7 Amino acid sequence:
MLYPEKFQGIGISNAKDWKHPKLVSFDPKPFGDHDVDVEIEACGICGSDFHIAVGNW
GPVPENQILGHEIIGRVVKVGSKCHTGVKIGDRVGVGAQALACFECERCKSDNEQYC
TNDHVLTMWTPYKDGYISQGGFASHVRLHEHFAIQIPENIPSPLAAPLLCGGITVFSPL
LRNGCGPGKRVGIVGIGGIGHNIGILLAKAMGAEVYAFSRGHSKREDSMKLGADHYI
AMLEDKGWTEQYSNALDLLVVCSSSLSKVNFDSIVKIMKIGGSIVSIAAPEVNEKLVL
KPLGLMGVSISSSAIGSRKEIEQLLKLVSEKNVKIWVEKLPISEEGVSHAFTRMESGDV
KYRFTLVDYDKKFHK*
SEQ ID NO: 51-adh7 Nucleotide sequence:
Atgctttacccagaaaaatttcagggcatcggtatttccaacgcaaaggattggaagcatcctaaattagtgagttttgacccaaaaccc
tttggcgatcatgacgttgatgttgaaattgaagcctgtggtatctgcggatctgattttcatatagccgttggtaattggggtccagtccca
gaaaatcaaatccttggacatgaaataattggccgcgtggtgaaggttggatccaagtgccacactggggtaaaaatcggtgaccgtg
ttggtgttggtgcccaagccttggcgtgttttgagtgtgaacgttgcaaaagtgacaacgagcaatactgtaccaatgaccacgttttgac
tatgtggactccttacaaggacggctacatttcacaaggaggattgcctcccacgtgaggcttcatgaacactttgctattcaaatacca
gaaaatattccaagtccgctagccgctccattattgtgtggtggtattacagttttctctccactactaagaaatggctgtggtccaggtaa
gagggtaggtattgttggcatcggtggtattgggcatatggggattctgttggctaaagctatgggagccgaggtttatgcgttttcgcga
ggccactccaagcgggaggattctatgaaactcggtgctgatcactatattgctatgttggaggataaaggctggacagaacaatactc
taacgctttggaccttcttgtcgtttgctcatcatctttgtcgaaagttaattttgacagtatcgttaagattatgaagattggaggctccatcgt
ttcaattgctgctcctgaagttaatgaaaagcttgttttaaaaccgttgggcctaatgggagtatcaatctcaagcagtgctatcggatcta
ggaaggaaatcgaacaactattgaaattagtttccgaaaagaatgtcaaaatatgggtggaaaaacttccgatcagcgaagaaggcgt
cagccatgcctttacaaggatggaaagcggagacgtcaaatacagatttactttggtcgattatgataagaaattccataaatag
8. SFA1 from Saccharomyces cerevisae
SEQ ID NO: 52-SFA1 Amino acid sequence:
MSAATVGKPIKCIAAVAYDAKKPLSVEEITVDAPKAHEVRIKIEYTAVCHTDAYTLS
GSDPEGLFPCVLGHEGAGIVESVGDDVITVKPGDHVIALYTAECGKCKFCTSGKTNL
CGAVRATQGKGVMPDGTTRFHNAKGEDIYHFMGCSTFSEYTVVADVSVVAIDPKAP
LDAACLLGCGVTTGFGAALKTANVQKGDTVAVFGCGTVGLSVIQGAKLRGASKIIAI
DINNKKKQYCSQFGATDFVNPKEDLAKDQTIVEKLIEMTDGGLDFTFDCTGNTKIMR
DALEACHKGWGQSIIIGVAAAGEEISTRPFQLVTGRVWKGSAFGGIKGRSEMGGLIK
DYQKGALKVEEFITHRRPFKEINQAFEDLHNGDCLRTVLKSDEIK
SEQ ID NO: 53-sfa1 Nucleotide sequence:
Atgtccgccgctactgttggtaaacctattaagtgcattgctgctgttgcgtatgatgcgaagaaaccattaagtgttgaagaaatcacg
gtagacgccccaaaagcgcacgaagtacgtatcaaaattgaatatactgctgtatgccacactgatgcgtacactttatcaggctctgat
ccagaaggacttttcccttgcgttctgggccacgaaggagccggtatcgtagaatctgtaggcgatgatgtcataacagttaagcctggt
gatcatgttattgctttgtacactgctgagtgtggcaaatgtaagttctgtacttccggtaaaaccaacttatgtggtgctgttagagctactc
aagggaaaggtgtaatgcctgatgggaccacaagatttcataatgcgaaaggtgaagatatataccatttcatgggttgctctactttttcc
gaatatactgtggtggcagatgtctctgtggttgccatcgatccaaaagctcccttggatgctgcctgtttactgggttgtggtgttactact
ggttttggggcggctcttaagacagctaatgtgcaaaaaggcgataccgttgcagtatttggctgcgggactgtaggactctccgttatc
caaggtgcaaagttaaggggcgcttccaagatcattgccattgacattaacaataagaaaaaacaatattgttctcaatttggtgccacg
gattttgttaatcccaaggaagatttggccaaagatcaaactatcgttgaaaagttaattgaaatgactgatgggggtctggattttactttt
gactgtactggtaataccaaaattatgagagatgctttggaagcctgtcataaaggttggggtcaatctattatcattggtgtggctgccgc
tggtgaagaaatttctacaaggccgttccagctggtcactggtagagtgtggaaaggctctgcttttggtggcatcaaaggtagatctga
aatgggcggtttaattaaagactatcaaaaaggtgccttaaaagtcgaagaatttatcactcacaggagaccattcaaagaaatcaatca
agcctttgaagatttgcataacggtgattgcttaagaaccgtcttgaagtctgatgaaataaaatag
SEQ ID No: 54 IlvC amino acid sequence from E. coli Nissle
MANYFNTLNLRQQLAQLGKCRFMGRDEFADGASYLQGKKVVIVGCGAQGLNQGL
NMRDSGLDISYALRKEAIAEKRASWRKATENGFKVGTYEELIPQADLVVNLTPDKQ
HSDVVRTVQPLMKDGAALGYSHGFNIVEVGEQIRKDITVVMVAPKCPGTEVREEYK
RGFGVPTLIAVHPENDPKGEGMAIAKAWAAATGGHRAGVLESSFVAEVKSDLMGE
QTILCGMLQAGSLLCFDKLVEEGTDPAYAEKLIQFGWETITEALKQGGITLMMDRLS
NPAKLRAYALSEQLKEIMAPLFQKHMDDIISGEFSSGMMADWANDDKKLLTWREET
GKTAFETAPQYEGKIGEQEYFDKGVLMIAMVKAGVELAFETMVDSGIIEESAYYESL
HELPLIANTIARKRLYEMNVVISDTAEYGNYLFSYACVPLLKPFMAELQPGDLGKAIP
EGAVDNAQLRDVNEAIRSHAIEQVGKKLRGYMTDMKRIAVAG
SEQ ID 55: ilvC gene from E. coli Nissle nucleotide sequence
atggctaactacttcaatacactgaatctgcgccagcagttggcacagctgggcaaatgtcgctttatggggcgcgatgaattcgccga
tggcgcgagctaccttcagggtaaaaaagtagtcatcgtcggctgtggcgcacagggtctgaaccagggcctgaacatgcgtgattct
ggtctcgatatctcctacgctctgcgtaaagaagcgattgctgagaagcgcgcatcctggcgtaaagcaaccgaaaatggttttaaagt
gggtacttacgaagaactgatcccgcaggcggatctggtggttaacctgacgccggacaagcagcactctgatgtagtgcgcaccgt
acagccactgatgaaagacggcgcggcgctgggctactctcatggtttcaatatcgtagaagtgggtgagcagatccgtaaagacatc
accgtcgtaatggttgcgccgaaatgccctggcaccgaagtacgtgaagagtacaaacgtggattcggcgtaccgacgctgattgcc
gttcacccggaaaacgatccgaaaggcgaaggcatggcgatcgctaaagcatgggcggctgcaaccggcggtcaccgtgcgggc
gttctggaatcctctttcgttgcggaagtgaaatctgacctgatgggcgagcaaaccatcctgtgcggtatgttgcaagctggttctctgc
tgtgcttcgacaagctggtggaagaaggcaccgatccggcatacgcagaaaaactgattcagttcggttgggaaaccatcaccgaag
cgctgaaacagggcggcatcaccctgatgatggaccgtctttctaacccggcgaaactgcgtgcttacgcgctttctgagcaactgaa
agagatcatggcgccgctgttccagaaacatatggacgacatcatctccggcgaattctcctccggcatgatggctgactgggccaac
gacgataagaaactgctgacctggcgtgaagagactggcaaaaccgcattcgaaaccgcgccgcagtatgaaggcaaaatcggtga
acaggagtacttcgataaaggcgtactgatgatcgcgatggtaaaagcaggcgttgagttggcgtttgaaaccatggttgattccggca
tcatcgaagaatctgcttactatgaatcactgcacgaactgccgctgattgccaacaccatcgcccgtaagcgtctgtacgaaatgaac
gtggttatctccgatactgccgagtacggtaactatctgttctcttacgcttgtgtgccactgctgaaaccgtttatggcagagctgcaacc
gggcgacctgggtaaagctattccggaaggtgcggtagataacgcgcagctgcgtgatgtaaatgaagcgattcgcagccatgcgat
tgagcaggtaggtaagaaactgcgcggctatatgacggatatgaaacgtattgctgttgcgggttaa
L-amino acid deaminase L-AAD (from Proteus vulgaris)
SEQ ID NO: 56: Codon-optimized sequence:
ATGGCCATCAGTCGTCGCAAATTCATTATCGGTGGAACGGTCGTCGCCGTTGCCG
CCGGTGCGGGGATTTTGACCCCGATGCTGACGCGCGAAGGGCGCTTTGTGCCGG
GCACTCCACGCCACGGTTTCGTTGAAGGGACCGAGGGGGCTTTACCCAAACAAG
CGGACGTGGTGGTCGTAGGCGCTGGAATTCTTGGTATTATGACGGCCATTAATTT
GGTTGAGCGTGGGCTGTCAGTGGTAATTGTGGAGAAGGGCAATATCGCGGGAGA
ACAAAGCTCTCGCTTCTACGGACAGGCAATTAGCTATAAGATGCCAGATGAGAC
ATTTTTGCTGCACCATCTTGGGAAGCACCGCTGGCGTGAGATGAATGCGAAAGTA
GGGATTGATACAACGTACCGTACTCAAGGACGCGTGGAAGTACCGCTTGACGAG
GAAGATTTGGTAAACGTCCGCAAATGGATTGACGAACGTTCAAAAAATGTTGGA
TCTGACATTCCTTTTAAGACCCGCATTATCGAGGGGGCAGAATTAAATCAGCGTC
TGCGCGGCGCCACAACAGATTGGAAGATCGCTGGCTTCGAGGAGGACAGCGGGT
CATTCGATCCCGAGGTAGCGACCTTTGTAATGGCAGAGTACGCGAAGAAGATGG
GTGTTCGTATCTATACGCAATGCGCGGCCCGCGGTCTGGAAACCCAGGCCGGTGT
CATTTCAGATGTTGTCACGGAAAAAGGTGCGATTAAGACCTCCCAAGTGGTAGTG
GCTGGTGGGGTGTGGAGTCGTCTGTTCATGCAGAATTTAAACGTCGACGTCCCAA
CCCTTCCTGCGTATCAGTCACAGCAGTTGATTAGTGGTTCCCCTACCGCACCGGG
GGGGAACGTCGCATTACCTGGTGGAATCTTCTTCCGCGAACAGGCGGACGGGAC
ATACGCGACTTCTCCTCGTGTGATTGTTGCCCCAGTTGTGAAGGAGAGCTTCACT
TATGGTTACAAGTACTTACCATTATTAGCATTGCCTGATTTCCCTGTTCACATTAG
CCTGAATGAACAGTTAATTAATTCGTTTATGCAAAGTACCCACTGGAACTTAGAC
GAAGTGTCGCCGTTCGAACAATTTCGCAACATGACAGCATTACCTGACTTGCCCG
AACTTAACGCCAGCTTAGAAAAGTTAAAGGCAGAGTTCCCTGCTTTCAAAGAATC
CAAGTTGATCGACCAGTGGTCTGGAGCAATGGCAATTGCGCCCGACGAAAATCC
AATCATTTCCGAGGTGAAGGAGTACCCAGGTCTGGTAATTAACACGGCGACAGG
TTGGGGCATGACTGAAAGTCCAGTGTCTGCTGAACTTACCGCCGATCTTCTGCTG
GGGAAGAAGCCGGTGTTAGATCCTAAGCCATTCTCACTTTATCGCTTTTGA
SEQ ID NO: 57: Amino acid sequence:
MAISRRKFIIGGTVVAVAAGAGILTPMLTREGRFVPGTPRHGFVEGTEGALPKQADV
VVVGAGILGIMTAINLVERGLSVVIVEKGNIAGEQSSRFYGQAISYKMPDETFLLHHL
GKHRWREMNAKVGIDTTYRTQGRVEVPLDEEDLVNVRKWIDERSKNVGSDIPFKTR
IIEGAELNQRLRGATTDWKIAGFEEDSGSFDPEVATFVMAEYAKKMGVRIYTQCAAR
GLETQAGVISDVVTEKGAIKTSQVVVAGGVWSRLFMQNLNVDVPTLPAYQSQQLIS
GSPTAPGGNVALPGGIFFREQADGTYATSPRVIVAPVVKESFTYGYKYLPLLALPDFP
VHISLNEQLINSFMQSTHWNLDEVSPFEQFRNMTALPDLPELNASLEKLKAEFPAFKE
SKLIDQWSGAMAIAPDENPIISEVKEYPGLVINTATGWGMTESPVSAELTADLLLGKK
PVLDPKPFSLYRF*
Leucine dehydrogenase leuDH from Bacillus cereus:
SEQ ID NO: 58 Codon-optimized sequence:
ATGACTCTTGAAATCTTTGAATATTTAGAAAAGTACGACTACGAGCAGGTTGTAT
TTTGTCAAGACAAGGAGTCTGGGCTGAAGGCCATCATTGCCATCCACGACACAA
CCTTAGGCCCGGCGCTTGGCGGAACCCGCATGTGGACCTACGACTCCGAGGAGG
CGGCCATCGAGGACGCACTTCGTCTTGCTAAGGGTATGACCTATAAGAACGCGG
CAGCCGGTCTGAATCTGGGGGGTGCTAAGACTGTAATCATCGGTGATCCACGCA
AGGATAAGAGTGAAGCAATGTTTCGCGCTTTAGGGCGCTATATTCAGGGCTTGAA
CGGCCGCTACATTACCGCAGAAGACGTAGGGACAACAGTAGACGACATGGACAT
CATCCATGAGGAAACTGATTTCGTGACCGGTATTTCACCTTCATTCGGGTCATCC
GGTAACCCTTCCCCCGTAACAGCCTATGGGGTTTATCGCGGAATGAAGGCCGCAG
CCAAGGAGGCATTTGGCACTGACAATTTAGAAGGAAAAGTAATTGCTGTCCAAG
GCGTGGGCAATGTGGCCTACCATTTGTGTAAACACCTTCACGCGGAAGGTGCAA
AATTGATCGTTACGGATATTAACAAGGAGGCAGTCCAGCGCGCTGTAGAGGAAT
TTGGAGCATCGGCTGTGGAACCAAATGAGATCTACGGTGTAGAATGTGACATTTA
CGCTCCATGCGCACTTGGTGCCACGGTGAATGACGAGACCATCCCCCAACTTAAG
GCGAAGGTAATCGCTGGTTCAGCTAACAACCAATTAAAAGAGGACCGTCACGGA
GATATCATCCACGAAATGGGTATCGTGTACGCCCCCGATTATGTTATCAACGCGG
GCGGCGTAATCAACGTAGCCGATGAGCTTTATGGATACAACCGCGAACGTGCGC
TGAAACGCGTGGAAAGCATTTATGACACGATCGCAAAGGTAATCGAGATCAGTA
AGCGCGACGGCATTGCGACATACGTGGCAGCGGACCGTCTGGCCGAAGAACGCA
TCGCGAGTTTGAAGAATAGCCGTAGTACCTACTTGCGCAACGGGCACGATATTAT
CAGCCGTCGCtga
SEQ ID NO: 59 amino acid sequence:
MTLEIFEYLEKYDYEQVVFCQDKESGLKAIIAIHDTTLGPALGGTRMWTYDSEEAAIE
DALRLAKGMTYKNAAAGLNLGGAKTVIIGDPRKDKSEAMFRALGRYIQGLNGRYIT
AEDVGTTVDDMDIIHEETDFVTGISPSFGSSGNPSPVTAYGVYRGMKAAAKEAFGTD
NLEGKVIAVQGVGNVAYHLCKHLHAEGAKLIVTDINKEAVQRAVEEFGASAVEPNE
IYGVECDIYAPCALGATVNDETIPQLKAKVIAGSANNQLKEDRHGDIIHEMGIVYAPD
YVINAGGVINVADELYGYNRERALKRVESIYDTIAKVIEISKRDGIATYVAADRLAEE
RIASLKNSRSTYLRNGHDIISRR*
Alcohol dehydrogenase YqhD from E. coli:
SEQ ID NO: 60 Nucleotide sequence:
Atgaacaactttaatctgcacaccccaacccgcattctgtaggtaaaggcgcaatcgctggatacgcgaacaaattcctcacgatgct
cgcgtattgattacctacggcggcggcagcgtgaaaaaaaccggcgactcgatcaagactggatgccctgaaaggcatggacgtac
tggaataggcggtattgaaccaaacccggcttatgaaacgctgatgaacgccgtgaaactggacgcgaacagaaagtgacgacctg
ctggcggaggcggcggactgtactggacggcaccaaatttatcgccgcagcggctaactatccggaaaatatcgatccgtggcaca
actgcaaacgggcggtaaagagattaaaagcgccatcccgatgggctgtgtgctgacgctgccagcaaccggttcagaatccaacg
caggcgcggtgatctcccgtaaaaccacaggcgacaagcaggcgaccattctgcccatgacagcccgtatttgccgtgctcgatcc
ggatatacctacaccctgccgccgcgtcaggtggctaacggcgtagtggacgcctagtacacaccgtggaacagtatgttaccaaac
cggttgatgccaaaattcaggaccgatcgcagaaggcattagctgacgctgatcgaagatggtccgaaagccctgaaagagccaga
aaactacgatgtgcgcgccaacgtcatgtgggcggcgactcaggcgctgaacggatgatcggcgctggcgtaccgcaggactggg
caacgcatatgctgggccacgaactgactgcgatgcacggtctggatcacgcgcaaacactggctatcgtcctgcctgcactgtggaa
tgaaaaacgcgataccaagcgcgctaagctgctgcaatatgctgaacgcgtctggaacatcactgaaggttcagacgatgagcgtatt
gacgccgcgattgccgcaacccgcaatactagagcaattaggcgtgctgacccacctctccgactacggtctggacggcagctccat
cccggctagctgaaaaaactggaagagcacggcatgacccaactgggcgaaaatcatgacattacgctggatgtcagccgccgtat
atacgaagccgcccgctaa
SEQ ID NO: 61 amino acid sequence:
MNNFNLHTPTRILFGKGAIAGLREQIPHDARVLITYGGGSVKKTGVLDQVLDALKGM
DVLEFGGIEPNPAYETLMNAVKLVREQKVTFLLAVGGGSVLDGTKFIAAAANYPENI
DPWHILQTGGKEIKSAIPMGCVLTLPATGSESNAGAVISRKTTGDKQAFHSAHVQPV
FAVLDPVYTYTLPPRQVANGVVDAFVHTVEQYVTKPVDAKIQDRFAEGILLTLIEDG
PKALKEPENYDVRANVMWAATQALNGLIGAGVPQDWATHMLGHELTAMHGLDH
AQTLAIVLPALWNEKRDTKRAKLLQYAERVWNITEGSDDERIDAAIAATRNFFEQLG
VLTHLSDYGLDGSSIPALLKKLEEHGMTQLGENHDITLDVSRRIYEAAR*
Aldehyde dehydrogenase PadA from E. coli:
SEQ ID NO: 62: Nucleotide sequence:
ATGACAGAGCCGCATGTAGCAGTATTAAGCCAGGTCCAACAGTTTCTCGATCGTC
AACACGGTCTTTATATTGATGGTCGTCCTGGCCCCGCACAAAGTGAAAAACGGTT
GGCGATCTTTGATCCGGCCACCGGGCAAGAAATTGCGTCTACTGCTGATGCCAAC
GAAGCGGATGTAGATAACGCAGTCATGTCTGCCTGGCGGGCCTTTGTCTCGCGTC
GCTGGGCCGGGCGATTACCCGCAGAGCGTGAACGTATTCTGCTACGTTTTGCTGA
TCTGGTGGAGCAGCACAGTGAGGAGCTGGCGCAACTGGAAACCCTGGAGCAAGG
CAAGTCAATTGCCATTTCCCGTGCTTTTGAAGTGGGCTGTACGCTGAACTGGATG
CGTTATACCGCCGGGTTAACGACCAAAATCGCGGGTAAAACGCTGGACTTGTCG
ATTCCCTTACCCCAGGGGGCGCGTTATCAGGCCTGGACGCGTAAAGAGCCGGTTG
GCGTAGTGGCGGGAATTGTGCCATGGAACTTTCCGTTGATGATTGGTATGTGGAA
GGTGATGCCAGCACTGGCAGCAGGCTGTTCAATCGTGATTAAGCCTTCGGAAACC
ACGCCACTGACGATGTTGCGCGTGGCGGAACTGGCCAGCGAGGCTGGTATCCCT
GATGGCGTTTTTAATGTCGTCACCGGGTCAGGTGCTGTATGCGGCGCGGCCCTGA
CGTCACATCCTCATGTTGCGAAAATCAGTTTTACCGGTTCAACCGCGACGGGAAA
AGGTATTGCCAGAACTGCTGCTGATCACTTAACGCGTGTAACGCTGGAACTGGGC
GGTAAAAACCCGGCAATTGTATTAAAAGATGCTGATCCGCAATGGGTTATTGAA
GGCTTGATGACCGGAAGCTTCCTGAATCAAGGGCAAGTATGCGCCGCCAGTTCG
CGAATTTATATTGAAGCGCCGTTGTTTGACACGCTGGTTAGTGGATTTGAGCAGG
CGGTAAAATCGTTGCAAGTGGGACCGGGGATGTCACCTGTTGCACAGATTAACC
CTTTGGTTTCTCGTGCGCACTGCGACAAAGTGTGTTCATTCCTCGACGATGCGCA
GGCACAGCAAGCAGAGCTGATTCGCGGGTCGAATGGACCAGCCGGAGAGGGGT
ATTATGTTGCGCCAACGCTGGTGGTAAATCCCGATGCTAAATTGCGCTTAACTCG
TGAAGAGGTGTTTGGTCCGGTGGTAAACCTGGTGCGAGTAGCGGATGGAGAAGA
GGCGTTACAACTGGCAAACGACACGGAATATGGCTTAACTGCCAGTGTCTGGAC
GCAAAATCTCTCCCAGGCTCTGGAATATAGCGATCGCTTACAGGCAGGGACGGT
GTGGGTAAACAGCCATACCTTAATTGACGCTAACTTACCGTTTGGTGGGATGAAG
CAGTCAGGAACGGGCCGTGATTTTGGCCCCGACTGGCTGGACGGTTGGTGTGAA
ACTAAGTCGGTGTGTGTACGGTATTAA
SEQ ID NO: 63 amino acid sequence:
MTEPHVAVLSQVQQFLDRQHGLYIDGRPGPAQSEKRLAIFDPATGQEIASTADANEA
DVDNAVMSAWRAFVSRRWAGRLPAERERILLRFADLVEQHSEELAQLETLEQGKSI
AISRAFEVGCTLNWMRYTAGLTTKIAGKTLDLSIPLPQGARYQAWTRKEPVGVVAGI
VPWNFPLMIGMWKVMPALAAGCSIVIKPSETTPLTMLRVAELASEAGIPDGVFNVVT
GSGAVCGAALTSHPHVAKISFTGSTATGKGIARTAADHLTRVTLELGGKNPAIVLKD
ADPQWVIEGLMTGSFLNQGQVCAASSRIYIEAPLFDTLVSGFEQAVKSLQVGPGMSP
VAQINPLVSRAHCDKVCSFLDDAQAQQAELIRGSNGPAGEGYYVAPTLVVNPDAKL
RLTREEVFGPVVNLVRVADGEEALQLANDTEYGLTASVWTQNLSQALEYSDRLQAG
TVWVNSHTLIDANLPFGGMKQSGTGRDFGPDWLDGWCETKSVCVRY*
BCAA transporter BrnQ from E. coli:
SEQ ID NO: 64 Nucleotide sequence:
atgacccatcaattaagatcgcgcgatatcatcgctctgggctttatgacatttgcgttgttcgtcggcgcaggtaacattattttccctcca
atggtcggcttgcaggcaggcgaacacgtctggactgcggcattcggcttcctcattactgccgttggcctaccggtattaacggtagt
ggcgctggcaaaagttggcggcggtgttgacagtctcagcacgccaattggtaaagtcgctggcgtactgctggcaacagtttgttac
ctggcggtggggccgctttttgctacgccgcgtacagctaccgtttcttttgaagtgggcattgcgccgctgacgggtgattccgcgctg
ccgctgtttatttacagcctggtctatttcgctatcgttattctggtttcgctctatccgggcaagctgctggataccgtgggcaacttccttg
cgccgctgaaaattatcgcgctggtcatcctgtctgttgccgcaattatctggccggcgggttctatcagtacggcgactgaggcttatc
aaaacgctgcgttttctaacggcttcgtcaacggctatctgaccatggatacgctgggcgcaatggtgtttggtatcgttattgttaacgcg
gcgcgttctcgtggcgttaccgaagcgcgtctgctgacccgttataccgtctgggctggcctgatggcgggtgttggtctgactctgctg
tacctggcgctgttccgtctgggttcagacagcgcgtcgctggtcgatcagtctgcaaacggtgcggcgatcctgcatgcttacgttca
gcatacctttggcggcggcggtagcttcctgctggcggcgttaatcttcatcgcctgcctggtcacggcggttggcctgacctgtgcttg
tgcagaattcttcgcccagtacgtaccgctctcttatcgtacgctggtgtttatcctcggcggcttctcgatggtggtgtctaacctcggctt
gagccagctgattcagatctctgtaccggtgctgaccgccatttatccgccgtgtatcgcactggttgtattaagttttacacgctcatggt
ggcataattcgtcccgcgtgattgctccgccgatgtttatcagcctgctttttggtattctcgacgggatcaaggcatctgcattcagcgat
atcttaccgtcctgggcgcagcgtttaccgctggccgaacaaggtctggcgtggttaatgccaacagtggtgatggtggttctggccatt
atctgggatcgtgcggcaggtcgtcaggtgacctccagcgctcactaa
SEQ ID NO: 65 amino acid sequence:
MTHQLRSRDIIALGFMTFALFVGAGNIIFPPMVGLQAGEHVWTAAFGFLITAVGLPVL
TVVALAKVGGGVDSLSTPIGKVAGVLLATVCYLAVGPLFATPRTATVSFEVGIAPLT
GDSALPLFIYSLVYFAIVILVSLYPGKLLDTVGNFLAPLKIIALVILSVAAIIWPAGSIST
ATEAYQNAAFSNGFVNGYLTMDTLGAMVFGIVIVNAARSRGVTEARLLTRYTVWA
GLMAGVGLTLLYLALFRLGSDSASLVDQSANGAAILHAYVQHTFGGGGSFLLAALIF
IACLVTAVGLTCACAEFFAQYVPLSYRTLVFILGGFSMVVSNLGLSQLIQISVPVLTAI
YPPCIALVVLSFTRSWWHNSSRVIAPPMFISLLFGILDGIKASAFSDILPSWAQRLPLAE
QGLAWLMPTVVMVVLAIIWDRAAGRQVTSSAH*
Isovaleryl-CoA synthetase LbuL from Streptomyces lividans
SEQ ID NO: 66: amino acid sequence:
MTAPAPQPSYAHGTSTTPLLGDTVGANLGRAIAAHPDREALVDVPSGRRWTYAEFG
AAVDELARGLLAKGVTRGDRVGIWAVNCPEWVLVQYATARIGVIMVNVNPAYRAH
ELEYVLQQSGISLLVASLAHKSSDYRAIVEQVRGRCPALRETVYIGDPSWDALTAGA
AAVEQDRVDALAAELSCDDPVNIQYTSGTTGFPKGATLSHHNILNNGYWVGRTVGY
TEQDRVCLPVPFYHCFGMVMGNLGATSHGACIVIPAPSFEPAATLEAVQRERCTSLY
GVPTMFIAELNLPDFASYDLTSLRTGIMAGSPCPVEVMKRVVAEMHMEQVSICYGM
TETSPVSLQTRMDDDLEHRTGTVGRVLPHIEVKVVDPVTGVTLPRGEAGELRTRGYS
VMLGYWEEPGKTAEAIDPGRWMHTGDLAVMREDGYVEIVGRIKDMIIRGGENIYPR
EVEEFLYAHPKIADVQVVGVPHERYGEEVLACVVVRDAADPLTLEELRAYCAGQLA
HYKVPSRLQLLDSFPMTVSGKVRKVELRERYGTRP*
SEQ ID NO: 67: Codon-optimized nucleotide sequence:
atgactgcaccagcacctcaaccctcttatgcacatggcacactaccactccgcttcaggtgatacggtgggggcaaacctgggtcgt
gccatcgcggctcatcccgatcgtgaggcactggtcgatgtacccagcggacgccgaggacttacgcagagtaggcgcggccgta
gatgaattagcacgcggcctgttagccaaaggggtaactcgcggtgaccgtgtgggtatagggctgtgaactgtcccgaatgggatt
ggtgcaatacgctacagcccgtattggggtaatcatggttaatgtaaatcccgcttatcgcgcccacgagcttgaatatgtactgcaaca
gagtggcataccttattagtggcttcacttgcacacaaaagttcagattaccgcgcaattgtggagcaagtgcgcggccgctgtcccgc
cttacgtgaaactgtgtacatcggtgatccatcatgggatgccttgactgcaggcgcagcggctgtcgaacaagatcgtgagacgctct
ggcggcggagcatcatgtgacgaccctgtcaacattcagtacactagcggtacgactggattccgaaaggagcaacattatctcacc
ataacatcttgaacaacggttattgggtagggcgcacagtcggctacactgagcaagaccgtgtctgcttaccagtcccgactatcatt
gctagggatggtgatgggaaatcaggagccacatcccatggggcctgtattgtgatcccggccccctccacgagcctgccgcgact
ttagaagctgacagcgcgaacgagtacaagcctgtacggcgacccacaatgatattgcggagcttaacctgccggactagcctcat
acgatttgacgagcctgcgcactggcatcatggcagggtcgccctgcccagtagaagtcatgaagcgtgtcgagctgagatgcatat
ggagcaggtctcgatagttatggtatgacggagaccagtcccgtgagtcacaaactcgcatggacgacgacttagaacaccgtacag
gtacggtcggtcgtgtacttccgcacattgaagtcaaagtagtggaccccgtgacaggtgtaacccaccccgcggggaggcagggg
agcttcgcactcgtggatacagcgtaatgctgggttattgggaggaacctggcaagacggctgaggctatcgatccgggtcgaggat
gcacacaggcgatcagcggtgatgcgtgaagatgggtatgagagattgagggcgcatcaaggacatgattattcgcggcggtgaa
aacatttatcctcgcgaggagaagaattatatatgcacacccaaagatcgcagacgtacaggtagtcggcgtgccacatgagcgttat
ggagaagaggtactggcgtgcgagtcgttcgcgacgcggccgacccactgaccctggaagaattacgcgcctactgtgcaggcca
gcttgctcattataaagtcccacgcgatacaacttaggattcgaccctatgaccgtgtcaggaaaggtacgtaaggagagttacgtga
gcgctacgggacacgcccgtga
LiuABCDE operon from Pseudomonas aeruginosa:
Amino acid sequences:
SEQ ID NO: 68: liuA:
MTYPSLNFALGETIDMLRDQVRGFVAAELQPRAAQIDQDNQFPMDMWRKFGEMGL
LGITVDEEYGGSALGYLAHAVVMEEISRASASVALSYGAHSNLCVNQIKRNGNAEQ
KARYLPALVSGEHIGALAMSEPNAGSDVVSMKLRADRVGDRFVLNGSKMWITNGP
DAHTYVIYAKTDADKGAHGITAFIVERDWKGFSRGPKLDKLGMRGSNTCELIFQDV
EVPEENVLGAVNGGVKVLMSGLDYERVVLSGGPVGIMQACMDVVVPYIHDRRQFG
QSIGEFQLVQGKVADMYTALNASRAYLYAVAAACDRGETTRKDAAGVILYSAERA
TQMALDAIQILGGNGYINEFPTGRLLRDAKLYEIGAGTSEIRRMLIGRELFNETR*
SEQ ID NO: 69 LiuB:
MAILHTQINPRSAEFAANAATMLEQVNALRTLLGRIHEGGGSAAQARHSARGKLLV
RERINRLLDPGSPFLELSALAAHEVYGEEVAAAGIVAGIGRVEGVECMIVGNDATVK
GGTYYPLTVKKHLRAQAIALENRLPCIYLVDSGGANLPRQDEVFPDREHFGRIFFNQ
ANMSARGIPQIAVVMGSCTAGGAYVPAMSDETVMVREQATIFLAGPPLVKAATGEV
VSAEELGGADVHCKVSGVADHYAEDDDHALAIARRCVANLNWRKQGQLQCRAPR
APLYPAEELYGVIPADSKQPYDVREVIARLVDGSEFDEFKALFGTTLVCGFAHLHGY
PIAILANNGILFAEAAQKGAHFIELACQRGIPLLFLQNITGFMVGQKYEAGGIAKHGA
KLVTAVACARVPKFTVLIGGSFGAGNYGMCGRAYDPRFLWMWPNARIGVMGGEQ
AAGVLAQVKREQAERAGQQLGVEEEAKIKAPILEQYEHQGHPYYSSARLWDDGVID
PAQTREVLALALSAALNAPIEPTAFGVFRM*
SEQ ID NO: 70: LiuC:
MSEFQTIQLEIDPRGVATLWLDRAEKNNAFNAVVIDELLQAIDRVGSDPQVRLLVLR
GRGRHFCGGADLAWMQQSVDLDYQGNLADAQRIAELMTHLYNLPKPTLAVVQGA
VFGGGVGLVSCCDMAIGSDDATFCLSEVRIGLIPATIAPFVVKAIGQRAARRYSLTAE
RFDGRRASELGLLSESCPAAELESQAEAWIANLLQNSPRALVACKALYHEVEAAELS
PALRRYTEAAIARIRISPEGQEGLRAFLEKRTPAWRNDA*
SEQ ID NO: 71 LiuD:
MNPDYRSIQRLLVANRGEIACRVMRSARALGIGSVAVHSDIDRHARHVAEADIAVDL
GGAKPADSYLRGDRIIAAALASGAQAIHPGYGFLSENADFARACEEAGLLFLGPPAA
AIDAMGSKSAAKALMEEAGVPLVPGYHGEAQDLETFRREAGRIGYPVLLKAAAGGG
GKGMKVVEREAELAEALSSAQREAKAAFGDARMLVEKYLLKPRHVEIQVFADRHG
HCLYLNERDCSIQRRHQKVVEEAPAPGLGAELRRAMGEAAVRAAQAIGYVGAGTV
EFLLDERGQFFFMEMNTRLQVEHPVTEAITGLDLVAWQIRVARGEALPLTQEQVPLN
GHAIEVRLYAEDPEGDFLPASGRLMLYREAAAGPGRRVDSGVREGDEVSPFYDPML
AKLIAWGETREEARQRLLAMLAETSVGGLRTNLAFLRRILGHPAFAAAELDTGFIAR
HQDDLLPAPQALPEHFWQAAAEAWLQSEPGHRRDDDPHSPWSRNDGWRSALARES
DLMLRCRDERRCVRLRHASPSQYRLDGDDLVSRVDGVTRRSAALRRGRQLFLEWE
GELLAIEAVDPIAEAEAAHAHQGGLSAPMNGSIVRVLVEPGQTVEAGATLVVLEAM
KMEHSIRAPHAGVVKALYCSEGELVEEGTPLVELDENQA*
SEQ ID NO: 72 LiuE:
MNLPKKVRLVEVGPRDGLQNEKQPIEVADKIRLVDDLSAAGLDYIEVGSFVSPKWVP
QMAGSAEVFAGIRQRPGVTYAALAPNLKGFEAALESGVKEVAVFAAASEAFSQRNI
NCSIKDSLERFVPVLEAARQHQVRVRGYISCVLGCPYDGDVDPRQVAWVARELQQ
MGCYEVSLGDTIGVGTAGATRRLIEAVASEVPRERLAGHFHDTYGQALANIYASLLE
GIAVFDSSVAGLGGCPYAKGATGNVASEDVLYLLNGLEIHTGVDMHALVDAGQRIC
AVLGKSNGSRAAKALLAKA**
SEQ ID NO: 73 liuABCDE codon optimized sequence:
atgacttacccgtccctgaattagcgctgggcgaaaccattgacatgagcgcgaccaagttcgtggcttcgagcagcggaactgcaa
cctcgcgcggctcaaattgaccaggataatcagtaccgatggatatgtggcgtaagttcggtgagatggggctcttaggtattacggtt
gatgaggaatacggaggtagcgcgctcggttacttagcccatgcggtcgtaatggaagaaatacccgtgcctctgcgagcgtagcgc
tgtcttatggtgcgcattcaaacctgtgcgttaaccagatcaaacgcaatggtaacgctgaacagaaagcgcgttatctgccggctagg
tgtccggcgaacacattggcgccctcgctatgtcggaacctaacgcagggtcggatgtggtgtctatgaaactgcgcgcggatcgcgt
tggcgatcgatcgtgctgaatggaccaaaatgtggatcaccaacgggcctgatgcacatacgtatgtgatctacgctaaaaccgacgc
agataaaggggcccatggcatcaccgcatttattgagagcgtgactggaaagggatagccgtggcccaaaactggataaactcggt
atgcgtggttcaaatacatgtgaactgattaccaagacgtcgaagtccccgaagaaaatgtgctgggtgcagtgaatgggggggtcaa
agtgttaatgtctggtctcgattatgaacgtgtagtgctgagcggtggtccggttggtattatgcaagcctgtatggacgtggtagtgccg
tacattcatgatcgccgccagttcggccagtcgatcggagaatttcagctggtgcagggtaaggagcggacatgtataccgctctgaat
gcactcgtgcgtacttgtatgctgtcgctgcagcctgcgatcgtggagaaacgactcgcaaagacgctgctggtgtgattctctacagc
gcagaacgtgctacccaaatggcacttgacgcgatccagatcagggaggcaatgggtatatcaatgagaccccacgggccgcctg
ctgcgcgatgcgaagctgtatgagatcggcgcgggtacgagcgaaatccgccgtatgttaatcggtcgtgaattatttaacgagactcg
ctgaagcctcgctcacccggccataccgccagggagagggcattccattgcatcgacaggcgcatcgccaggtcgggagcgggc
gccaaccgcaccgcccacctcgacacggagccaccgccatggccatcatcacacgcagattaacccgcgactgctgaattcgcg
gcgaatgccgcgaccatgctggagcaagttaacgcattgcgtacgctccaggtcgcatccacgaaggtggtggacggcggctcag
gctcgccattcggcacgtggcaaattgttggttcgcgaacgcatcaaccgcctgctggaccccggtagcccgtttttggagttgagcgc
gttagcagctcatgaggtgtatggggaagaagtcgcagcagcaggtatcgtggccgggatcgggcgtgtagaaggagtagaatgtat
gatcgaggtaatgatgccactgtgaaaggaggtacgtattacccgctgaccgtgaagaagcatctgcgcgcccaagcaatcgcatta
gaaaatcgtagccgtgtatctatctggtcgattcgggtggcgccaatctgcctcgccaggacgaggtctaccggatcgcgagcatttc
ggccgcatctattcaaccaagccaatatgagcgcccgcggtatcccgcagattgcggtggtaatgggctcatgtactgcgggtggcg
cctatgtcccggccatgtccgatgaaactgtgatggtccgtgagcaggcgacgatcacctggctggaccgcctctcgtgaaagcggc
cacgggtgaagtggatcagcagaggaattgggtggcgccgacgtgcattgtaaagtgtcaggcgtggcggaccactatgccgaag
atgatgaccatgcattggcgattgcgcgtcgctgtgagcgaatttaaattggcgcaaacagggtcagcttcagtgccgtgcgccgcgt
gctccgctgtatccggcggaagaactgtatggtgtgattccggcggatagcaaacagccgtatgatgtgcgcgaggtcattgcacgcc
tggagatggatctgaatttgatgaattcaaggcgctgacggaaccaccctggtgtgcggctagcacacctgcatggctacccaattgc
cattctcgcaaataatggcattctgacgcggaggcggcccagaaaggggcccatttcattgaactggcctgccaacgcggtattccatt
actgttcctgcaaaatatcaccggcttcatggttggtcagaagtatgaagctggcggtattgccaagcatggcgcgaaactggtcaccg
cggtcgcctgcgcccgcgtgccgaaatttacagtgctgattggcggaagatcggggcagggaactacggaatgtgtggtcgcgcgt
acgatccgcgcacctctggatgtggccgaatgcacgcattggcgtgatgggcggcgagcaggctgccggcgtcctggcacaggtc
aaacgtgagcaagcggaacgcgctggccaacagctgggggtggaggaagaagcgaaaattaaagcgccgatccttgaacagtatg
aacatcagggccatccgtactattcgtcagcacgtagtgggacgatggcgtcattgatcctgcccagacacgcgaagtccagcgctg
gcgctgagtgcggcgcttaacgctccgatcgaaccaactgcattcggtgtatttcgcatgtgacgagtagaccagcatgagcgaatttc
agacgatccagctggaaattgatccacgtggagtggcaaccctgtggctggaccgtgctgaaaaaaataacgcatttaacgccgtcgt
gatcgatgaactgctgcaggcgatcgaccgcgtaggcagcgacccccaggtccgtagctggtcttgcgtgggcgtggccgtcatttc
tgtggcggcgccgacctggcgtggatgcagcagtctgagacctggattatcagggtaaccagctgacgcccagcgcatcgcagag
ctcatgacccacttgtataatctgcccaaacctactttagcggtagttcaaggcgcagattcggcggcggggtcggtaggtgagctgct
gcgacatggcaattggtagtgatgacgccactattgatgtcagaggtacgcattgggctgattccagcaaccatcgccccgttcgtgg
tgaaagctattggtcaacgcgcagcgcgccgttattcactgactgctgaacgattgatgggcgccgcgcgtccgaactgggactgctt
agcgagtcttgcccggccgcagaactggaatcccaagcggaagcatggatcgcgaatcactccagaactctccacgtgcactcgtg
gcatgtaaagcgctgtatcacgaggtagaagcggctgaactgtcccctgcactgcgtcgctatacggaagccgcaattgcacgtatcc
gtatttcaccagaaggtcaagaaggcttgcgtgccatttagaaaaacgcacaccggcgtggagaaacgacgcatgaacccggacta
ccgttcaattcagcgtctcttagtagctaaccgtggcgagattgcctgtcgcgtaatgcgttcggcccgcgcgttaggtattggatcagtt
gcagttcattcggatatcgaccgccacgcacgtcacgtggctgaagctgatattgcggagacctgggcggcgccaaaccggcagatt
cgtatctgcgtggcgaccgtatcattgcagctgcactggcttcaggagcccaggccattcatccggggtatggctactgtctgagaatg
ctgattagcccgcgcgtgcgaagaagcaggatactgatagggcccaccggctgcggcaattgatgctatggggtctaagtcagcg
gcgaaagctttgatggaagaggcgggagtccccctggttccaggttaccacggtgaagcgcaggacttggaaacctttcgtcgcgag
gccggacgcatcggctatcccgtgctcttaaaggccgcggccggtggcggcggaaaagggatgaaagtcgtggaacgcgaggcc
gagctcgcagaagcgctgtccagcgcccaacgcgaagccaaagcggcctaggcgatgcgcgcatgctggtggagaagtatagtta
aaaccgcgtcacgtcgaaattcaggtattgcagatcgtcatggtcactgatatacctcaacgaacgtgactgacgatccaacgtcgcc
atcaaaaagagtagaagaagcgccggctcccggtagggcgcggaactgcgtcgtgccatgggcgaagcggccgttcgcgcagc
gcaagcgatcggctatgtgggggcgggcactgtagagtactcctggacgagcgcggtcaattcttattatggaaatgaacactcgcct
gcaggagaacaccctgtaactgaggccatcactggtctcgatttagtcgcgtggcagatccgtgtggcgcgtggtgaagcccaccgt
tgactcaagaacaagtaccgctgaacgggcacgcgatcgaagtccgcctgtacgcggaagaccctgaaggggattacttccggcaa
gtggacgcctgatgctgtatcgtgaagccgctgcaggtccgggccgccgcgtggattcgggagtccgtgagggcgacgaagtcag
ccccactacgatccgatgctggcaaaattgatcgcatggggggaaacccgtgaggaagctcgccaacgcctgctcgccatgaggc
cgagacctcggtcgggggcttgcgtacgaacctggcttattacgtcgtatcttaggccatcccgcattgccgccgctgaactggatac
cgggacattgctcgtcatcaagatgacctgctgccagcaccccaggctctgccagaacacactggcaagcagcagcagaagatgg
ctgcaaagcgaacctggtcatcgtcgcgatgacgatccgcattccccaggagccgtaacgatggaggcgctctgctaggcacgcg
aatctgatctgatgctgcgctgtcgcgatgaacgccgagtgtgcgtctgcgccatgatccccatctcaatatcgtcttgacggtgatgat
ctggtatcccgtgttgatggcgttacccgccgctccgcagcgttgcgtcgcggccgccagctgttcttagaatgggaaggtgaactgtt
agcgatcgaagctgagatccgattgcagaagccgaagcggcgcatgcccatcaaggcggatgagcgcgccaatgaacgggtctat
tgtacgcgactggttgagccggggcaaaccgtagaggcgggtgcgactcagtggattagaagcaatgaaaatggagcacagtatc
cgtgcgccacatgccggcgagttaaagcgctgtactgacagaaggagaattagttgaagagggcactcctctggagaactggacga
aaaccaggcctgacagccaagacgaggaacagcatgaacctgccgaagaaagttcgtctggagaagaggtccgcgcgatggactt
cagaacgaaaaacagccgatcgaagtggctgacaaaattcgccttgagatgacttgtcggcagccggcttagattatattgaagtggg
cagtttcgtctcaccgaaatgggttccgcagatggccgggagcgccgaagtgtttgctggcattcgtcaacgccctggcgtgacctac
gcggcactcgccccgaatttgaaaggcttcgaagcagctctggaatcgggtgtaaaagaagttgccgtgttcgcagcagcctccgaa
gcattctcccaacgcaacatcaactgctcgattaaagactcccttgagcgcttcgtcccggactggaagcggctcgccaacatcaggt
acgcgtccgcggatatatacctgcgtattgggagcccgtatgatggcgacgtagatccgcgccaggtcgcatgggtcgcacgtgaa
ctccagcagatgggctgctatgaggtcagtctcggcgatacaatcggtgtgggtaccgcgggcgcgacccgccgataattgaggcg
gtggcatctgaggaccccgcgaacgccagcaggccactacatgatacatatggacaggcgctggctaacatctatgcttattgctgg
agggcattgctgtatcgacagaccgtagctggcctcggtggctgcccatatgcaaaaggcgctaccggcaacgtcgcgagtgagg
atgtgctgtatcattaaatggtcttgaaattcataccggtgtggacatgcatgccctggtagacgcgggacagcgcatctgtgcggtgct
cggaaagtcgaatggctcccgtgctgcgaaggccctgctggccaaagcttaatga
SEQ ID NO: 91 Nucleotide sequence of the livKHMGF operon:
atgaaacggaatgcgaaaactatcatcgcagggatgattgcactggcaatttcacacaccgctatggctgacgatattaaagtcgccgt
tgtcggcgcgatgtccggcccgattgcccagtggggcgatatggaatttaacggcgcgcgtcaggcaattaaagacattaatgccaaa
gggggaattaagggcgataaactggaggcgtggaatatgacgacgcatgcgacccgaaacaagccgagcggtcgccaacaaaat
cgttaatgacggcattaaatacgttattggtcatctgtgacttcactacccagcctgcgtcagatatctatgaagacgaaggtattctgatg
atctcgccgggagcgaccaacccggagctgacccaacgcggttatcaacacattatgcgtactgccgggctggactcacccagggg
ccaacggcggcaaaatacattcttgagacggtgaagccccagcgcatcgccatcattcacgacaaacaacagtatggcgaagggct
ggcgcgttcggtgcaggacgggctgaaagcggctaacgccaacgtcgtatatcgacggtattaccgccggggagaaagatactc
cgcgctgatcgcccgcctgaaaaaagaaaacatcgacttcgatactacggcggttactacccggaaatggggcagatgctgcgcca
ggcccgaccgaggcctgaaaacccagatatggggccggaaggtgtgggtaatgcgtcgagtcgaacattgccggtgatgccgcc
gaaggcatgaggtcactatgccaaaacgctatgaccaggatccggcaaaccagggcatcgttgatgcgctgaaagcagacaagaa
agatccgtccgggccttatgtctggatcacctacgcggcggtgcaatctctggcgactgcccttgagcgtaccggcagcgatgagcc
gctggcgctggtgaaagatttaaaagctaacggtgcaaacaccgtgattgggccgctgaactgggatgaaaaaggcgatcttaaggg
atttgattaggtgtatccagtggcacgccgacggacatccacggcagccaagtgatcatcccaccgcccgtaaaatgcgggcgggt
ttagaaaggttaccttatgtctgagcagtattgtatacttgcagcagatgataacggcgtcacgctgggcagtacctacgcgctgatag
ccatcggctacaccatggatacggcattatcggcatgatcaacttcgcccacggcgaggatatatgattggcagctacgtctcatttatg
atcatcgccgcgctgatgatgatgggcattgataccggctggctgctggtagctgcgggattcgtcggcgcaatcgtcattgccagcg
cctacggctggagtatcgaacgggtggcttaccgcccggtgcgtaactctaagcgcctgattgcactcatctctgcaatcggtatgtcc
atcacctgcaaaactacgtcagcctgaccgaaggacgcgcgacgtggcgctgccgagcctgataacggtcagtgggtggtggggc
atagcgaaaacttctctgcctctattaccaccatgcaggcggtgatctggattgttaccttcctcgccatgctggcgctgacgattttcattc
gctattcccgcatgggtcgcgcgtgtcgtgcctgcgcggaagatctgaaaatggcgagtctgatggcattaacaccgaccgggtgat
tgcgctgacctagtgattggcgcggcgatggcggcggtggcgggtgtgctgctcggtcagactacggcgtcattaacccctacatcg
gattatggccgggatgaaagcattaccgcggcggtgctcggtgggattggcagcattccgggagcgatgattggcggcctgattct
ggggattgcggaggcgctctcactgcctatctgagtacggaatataaagatgtggtgtcattcgccctgctgattctggtgctgctggtg
atgccgaccggtattctgggtcgcccggaggtagagaaagtatgaaaccgatgcatattgcaatggcgctgctctctgccgcgatgac
tagtgctggcgggcgtattatgggcgtgcaactggagctggatggcaccaaactggtggtcgacacggcttcggatgtccgaggca
gtgggtgatatcggcacggcggtggtctattatccagatttgcgaccggattccagaaagggttgaaaagcgatccggaccgaag
tttattctgcccgccattgatggctccacggtgaagcagaaactgacctcgtggcgctgaggtgcttgcggtggcgtggccgatatgg
tacacgcgggacggtggatattgccaccctgaccatgatctacattatcctcggtctggggctgaacgtggagaggtctactggtctg
ctggtgctggggtacggcggtattacgccatcggcgcttacacattgcgctgctcaatcactattacggcttgggcttctggacctgcct
gccgattgctggattaatggcagcggcggcgggcacctgctcggattccggtgctgcgtagcgcggtgactatctggcgatcgttac
cctcggatcggcgaaattgtgcgcatattgctgctcaataacaccgaaattaccggcggcccgaacggaatcagtcagatcccgaaa
ccgacactatcggactcgagttcagccgtaccgctcgtgaaggcggctgggacacgttcagtaatactaggcctgaaatacgatccc
tccgatcgtgtcatcacctctacctggtggcgttgctgctggtggtgctaagcctgatgtcattaaccgcctgctgcggatgccgctggg
gcgtgcgtgggaagcgttgcgtgaagatgaaatcgcctgccgttcgctgggcttaagcccgcgtcgtatcaagctgactgcattacca
taagtgccgcgtagccggattgccggaacgctgatgcggcgcgtcagggctagtcagcccggaatcatcaccatgccgaatcgg
cgtagtgctggcgatagtggtgctcggcggtatgggctcgcaatttgcggtgattctggcggcaattagctggtggtgtcgcgcgagtt
gatgcgtgatttcaacgaatacagcatgttaatgctcggtggatgatggtgctgatgatgatctggcgtccgcagggcttgctgcccatg
acgcgcccgcaactgaagctgaaaaacggcgcagcgaaaggagagcaggcatgagtcagccattattatctgttaacggcctgatg
atgcgcttcggcggcctgctggcggtgaacaacgtcaatcttgaactgtacccgcaggagatcgtctcgttaatcggccctaacggtg
ccggaaaaaccacggtattaactgtctgaccggattctacaaacccaccggcggcaccatatactgcgcgatcagcacctggaaggt
ttaccggggcagcaaattgcccgcatgggcgtggtgcgcaccaccagcatgtgcgtctgaccgtgaaatgacggtaattgaaaacct
gctggtggcgcagcatcagcaactgaaaaccgggctgttctctggcctgttgaaaacgccatccttccgtcgcgcccagagcgaagc
gctcgaccgcgccgcgacctggcttgagcgcattggatgctggaacacgccaaccgtcaggcgagtaacctggcctatggtgacca
gcgccgtcttgagattgcccgctgcatggtgacgcagccggagattttaatgctcgacgaacctgcggcaggtcttaacccgaaagag
acgaaagagctggatgagctgattgccgaactgcgcaatcatcacaacaccactatcttgagattgaacacgatatgaagctggtgat
gggaatttcggaccgaatttacgtggtcaatcaggggacgccgctggcaaacggtacgccggagcagatccgtaataacccggacg
tgatccgtgcctatttaggtgaggcataagatggaaaaagtcatgagtcattgacaaagtcagcgcccactacggcaaaatccaggc
gctgcatgaggtgagcctgcatatcaatcagggcgagattgtcacgctgattggcgcgaacggggcggggaaaaccaccttgctcg
gcacgttatgcggcgatccgcgtgccaccagcgggcgaattgtgatgatgataaagacattaccgactggcagacagcgaaaatcat
gcgcgaagcggtggcgattgtcccggaagggcgtcgcgtcactcgcggatgacggtggaagagaacctggcgatgggcggatat
tgctgaacgcgaccagaccaggagcgcataaagtgggtgtatgagctgatccacgtctgcatgagcgccgtattcagcgggcgggc
accatgtccggcggtgaacagcagatgctggcgattggtcgtgcgctgatgagcaacccgcgtagctactgcttgatgagccatcgct
cggtatgcgccgattatcatccagcaaattacgacaccatcgagcagctgcgcgagcaggggatgactatattctcgtcgagcaga
acgccaaccaggcgctaaagctggcggatcgcggctacgtgctggaaaacggccatgtagtgctaccgatactggtgatgcgctgc
tggcgaatgaagcggtgagaagtgcgtatttaggcgggtaa
SEQ ID NO: 92 LivK
Amino acid sequence:
MKRNAKTIIAGMIALAISHTAMADDIKVAVVGAMSGPIAQWGDMEFNGARQAIKDI
NAKGGIKGDKLVGVEYDDACDPKQAVAVANKIVNDGIKYVIGHLCSSSTQPASDIYE
DEGILMISPGATNPELTQRGYQHIMRTAGLDSSQGPTAAKYILETVKPQRIAIIHDKQQ
YGEGLARSVQDGLKAANANVVFFDGITAGEKDFSALIARLKKENIDFVYYGGYYPE
MGQMLRQARSVGLKTQFMGPEGVGNASLSNIAGDAAEGMLVTMPKRYDQDPANQ
GIVDALKADKKDPSGPYVVVITYAAVQSLATALERTGSDEPLALVKDLKANGANTVI
GPLNWDEKGDLKGFDFGVFQWHADGSSTAAK*
SEQ ID NO: 93 Nucleotide sequence:
Atgaaacggaatgcgaaaactatcatcgcagggatgattgcactggcaatttcacacaccgctatggctgacgatattaaagtcgccg
agtcggcgcgatgtccggcccgattgcccagtggggcgatatggaatttaacggcgcgcgtcaggcaattaaagacattaatgccaa
agggggaattaagggcgataaactggaggcgtggaatatgacgacgcatgcgacccgaaacaagccgagcggtcgccaacaaaa
tcgttaatgacggcattaaatacgttattggtcatctgtgacttcactacccagcctgcgtcagatatctatgaagacgaaggtattctgat
gatctcgccgggagcgaccaacccggagctgacccaacgcggttatcaacacattatgcgtactgccgggctggactcacccaggg
gccaacggcggcaaaatacattcttgagacggtgaagccccagcgcatcgccatcattcacgacaaacaacagtatggcgaagggc
tggcgcgttcggtgcaggacgggctgaaagcggctaacgccaacgtcgtatatcgacggtattaccgccggggagaaagatactc
cgcgctgatcgcccgcctgaaaaaagaaaacatcgacttcgatactacggcggttactacccggaaatggggcagatgctgcgcca
ggcccgaccgaggcctgaaaacccagatatggggccggaaggtgtgggtaatgcgtcgagtcgaacattgccggtgatgccgcc
gaaggcatgaggtcactatgccaaaacgctatgaccaggatccggcaaaccagggcatcgttgatgcgctgaaagcagacaagaa
agatccgtccgggccttatgtctggatcacctacgcggcggtgcaatctctggcgactgcccttgagcgtaccggcagcgatgagcc
gctggcgctggtgaaagatttaaaagctaacggtgcaaacaccgtgattgggccgctgaactgggatgaaaaaggcgatcttaaggg
atttgattaggtgtatccagtggcacgccgacggacatccacggcagccaagtga
SEQ ID NO: 94 LivH
Amino acid sequence:
MSEQFLYFLQQMFNGVTLGSTYALIAIGYTMVYGIIGMINFAHGEVYMIGSYVSFMII
AALMMMGIDTGWLLVAAGFVGAIVIASAYGWSIERVAYRPVRNSKRLIALISAIGMS
IFLQNYVSLTEGSRDVALPSLFNGQWVVGHSENFSASITTMQAVIWIVTFLAMLALTI
FIRYSRMGRACRACAEDLKMASLLGINTDRVIALTFVIGAAMAAVAGVLLGQFYGVI
NPYIGFMAGMKAFTAAVLGGIGSIPGAMIGGLILGIAEALSSAYLSTEYKDVVSFALLI
LVLLVMPTGILGRPEVEKV*
SEQ ID NO: 95 Nucleotide sequence:
Atgtctgagcagtattgtatacttgcagcagatgataacggcgtcacgctgggcagtacctacgcgctgatagccatcggctacacc
atggatacggcattatcggcatgatcaacttcgcccacggcgaggatatatgattggcagctacgtctcatttatgatcatcgccgcgct
gatgatgatgggcattgataccggctggctgctggtagctgcgggattcgtcggcgcaatcgtcattgccagcgcctacggctggagt
atcgaacgggtggcttaccgcccggtgcgtaactctaagcgcctgattgcactcatctctgcaatcggtatgtccatcacctgcaaaact
acgtcagcctgaccgaaggttcgcgcgacgtggcgctgccgagcctgtttaacggtcagtgggtggtggggcatagcgaaaacttct
ctgcctctattaccaccatgcaggcggtgatctggattgttaccacctcgccatgctggcgctgacgattacattcgctattcccgcatg
ggtcgcgcgtgtcgtgcctgcgcggaagatctgaaaatggcgagtctgatggcattaacaccgaccgggtgattgcgctgacctagt
gattggcgcggcgatggcggcggtggcgggtgtgctgctcggtcagactacggcgtcattaacccctacatcggattatggccggg
atgaaagcattaccgcggcggtgctcggtgggattggcagcattccgggagcgatgattggcggcctgattctggggattgcggag
gcgctctcactgcctatctgagtacggaatataaagatgtggtgtcattcgccctgctgattctggtgctgctggtgatgccgaccggtat
tctgggtcgcccggaggtagagaaagtatga
LivM
SEQ ID NO: 96 Amino acid sequence:
MKPMHIAMALLSAAMFFVLAGVFMGVQLELDGTKLVVDTASDVRWQWVFIGTAV
VFFFQLLRPAFQKGLKSVSGPKFILPAIDGSTVKQKLFLVALLVLAVAWPFMVSRGT
VDIATLTMIYIILGLGLNVVVGLSGLLVLGYGGFYAIGAYTFALLNHYYGLGFWTCL
PIAGLMAAAAGFLLGFPVLRLRGDYLAIVTLGFGEIVRILLLNNTEITGGPNGISQIPKP
TLFGLEFSRTAREGGWDTFSNFFGLKYDPSDRVIFLYLVALLLVVLSLFVINRLLRMP
LGRAWEALREDEIACRSLGLSPRRIKLTAFTISAAFAGFAGTLFAARQGFVSPESFTFA
ESAFVLAIVVLGGMGSQFAVILAAILLVVSRELMRDFNEYSMLMLGGLMVLMMIWR
PQGLLPMTRPQLKLKNGAAKGEQA*
SEQ ID NO: 97 Nucleotide sequence:
atgaaaccgatgcatattgcaatggcgctgctctctgccgcgatgactagtgctggcgggcgtattatgggcgtgcaactggagctg
gatggcaccaaactggtggtcgacacggcttcggatgtccgaggcagtgggtgatatcggcacggcggtggtctattatccagatt
tgcgaccggctaccagaaagggagaaaagcgtaccggaccgaagatattctgcccgccattgatggctccacggtgaagcagaaa
ctgacctcgtggcgctgaggtgcttgcggtggcgtggccgatatggatcacgcgggacggtggatattgccaccctgaccatgatct
acattatcctcggtctggggctgaacgtggttgaggtctactggtctgctggtgctggggtacggcggatttacgccatcggcgcttac
acattgcgctgctcaatcactattacggcttgggatctggacctgcctgccgattgctggattaatggcagcggcggcgggatcctgc
tcggattccggtgctgcgtagcgcggtgactatctggcgatcgttaccctcggatcggcgaaattgtgcgcatattgctgctcaataac
accgaaattaccggcggcccgaacggaatcagtcagatcccgaaaccgacactatcggactcgagttcagccgtaccgctcgtgaa
ggcggctgggacacgttcagtaatactaggcctgaaatacgatccctccgatcgtgtcatcacctctacctggtggcgttgctgctggt
ggtgctaagcctgatgtcattaaccgcctgctgcggatgccgctggggcgtgcgtgggaagcgttgcgtgaagatgaaatcgcctgc
cgttcgctgggcttaagcccgcgtcgtatcaagctgactgcattaccataagtgccgcgtagccggattgccggaacgctgatgcgg
cgcgtcagggctagtcagcccggaatccttcaccatgccgaatcggcgtagtgctggcgatagtggtgctcggcggtatgggctcg
caatttgcggtgattctggcggcaattagctggtggtgtcgcgcgagttgatgcgtgatttcaacgaatacagcatgttaatgctcggtg
gatgatggtgctgatgatgatctggcgtccgcagggcttgctgcccatgacgcgcccgcaactgaagctgaaaaacggcgcagcga
aaggagagcaggcatga
LivG
SEQ ID NO: 98 Amino acid sequence:
MSQPLLSVNGLMMRFGGLLAVNNVNLELYPQEIVSLIGPNGAGKTTVFNCLTGFYKP
TGGTILLRDQHLEGLPGQQIARMGVVRTFQHVRLFREMTVIENLLVAQHQQLKTGLF
SGLLKTPSFRRAQSEALDRAATWLERIGLLEHANRQASNLAYGDQRRLEIARCMVTQ
PEILMLDEPAAGLNPKETKELDELIAELRNHHNTTILLIEHDMKLVMGISDRIYVVNQ
GTPLANGTPEQIRNNPDVIRAYLGEA*
SEQ ID NO: 100 Nucleotide sequence:
Atgagtcagccattattatctgttaacggcctgatgatgcgcttcggcggcctgctggcggtgaacaacgtcaatcttgaactgtacccg
caggagatcgtctcgttaatcggccctaacggtgccggaaaaaccacggtattaactgtctgaccggattctacaaacccaccggcgg
caccatatactgcgcgatcagcacctggaaggataccggggcagcaaattgcccgcatgggcgtggtgcgcaccaccagcatgtg
cgtctgttccgtgaaatgacggtaattgaaaacctgctggtggcgcagcatcagcaactgaaaaccgggctgttctctggcctgttgaa
aacgccatccaccgtcgcgcccagagcgaagcgctcgaccgcgccgcgacctggcttgagcgcattggatgctggaacacgcca
accgtcaggcgagtaacctggcctatggtgaccagcgccgtcttgagattgcccgctgcatggtgacgcagccggagattttaatgct
cgacgaacctgcggcaggtcttaacccgaaagagacgaaagagctggatgagctgattgccgaactgcgcaatcatcacaacacca
ctatcttgagattgaacacgatatgaagctggtgatgggaatttcggaccgaatttacgtggtcaatcaggggacgccgctggcaaac
ggtacgccggagcagatccgtaataacccggacgtgatccgtgcctatttaggtgaggcataa
LivF
SEQ ID NO: 101 Amino acid sequence:
MEKVMLSFDKVSAHYGKIQALHEVSLHINQGEIVTLIGANGAGKTTLLGTLCGDPRA
TSGRIVFDDKDITDWQTAKIMREAVAIVPEGRRVFSRMTVEENLAMGGFFAERDQFQ
ERIKWVYELFPRLHERRIQRAGTMSGGEQQMLAIGRALMSNPRLLLLDEPSLGLAPIII
QQIFDTIEQLREQGMTIFLVEQNANQALKLADRGYVLENGHVVLSDTGDALLANEA
VRSAYLGG*
SEQ ID NO: 102 Nucleotide sequence:
atggaaaaagtcatgagtccatgacaaagtcagcgcccactacggcaaaatccaggcgctgcatgaggtgagcctgcatatcaatca
gggcgagattgtcacgctgattggcgcgaacggggcggggaaaaccaccttgctcggcacgttatgcggcgatccgcgtgccacca
gcgggcgaattgtgatgatgataaagacattaccgactggcagacagcgaaaatcatgcgcgaagcggtggcgattgtcccggaag
ggcgtcgcgtcactcgcggatgacggtggaagagaacctggcgatgggcggatattgctgaacgcgaccagaccaggagcgcat
aaagtgggtgtatgagctgatccacgtctgcatgagcgccgtattcagcgggcgggcaccatgtccggcggtgaacagcagatgct
ggcgattggtcgtgcgctgatgagcaacccgcgtagctactgcttgatgagccatcgctcggtcttgcgccgattatcatccagcaaat
tacgacaccatcgagcagctgcgcgagcaggggatgactatctactcgtcgagcagaacgccaaccaggcgctaaagctggcgga
tcgcggctacgtgctggaaaacggccatgtagtgctttccgatactggtgatgcgctgctggcgaatgaagcggtgagaagtgcgtatt
taggcgggtaa

TABLE 27
Inducible promoter construct sequences

| | | SEQ |
|---|---|---|
| Description | Sequence | ID NO |
| Arabinose | CAGACATTGCCGTCACTGCGTCTTTTA | 103 |
| Promoter | CTGGCTCTTCTCGCTAACCCAACCGGT | |
| region | AACCCCGCTTATTAAAAGCATTCTGTA | |
| | ACAAAGCGGGACCAAAGCCATGACAAA | |
| | AACGCGTAACAAAAGTGTCTATAATCA | |
| | CGGCAGAAAAGTCCACATTGATTATTT | |
| | GCACGGCGTCACACTTTGCTATGCCAT | |
| | AGCATTTTTATCCATAAGATTAGCGGA | |
| | TCCAGCCTGACGCTTTTTTTCGCAACT | |
| | CTCTACTGTTTCTCCATACCTCTAGAA | |
| | ATAATTTTGTTTAACTTTAAGAAGGAG | |
| | ATATACAT | |
| AraC | TTATTCACAACCTGCCCTAAACTCGCT | 104 |
| (reverse | CGGACTCGCCCCGGTGCATTTTTTAAA | |
| orientation) | TACTCGCGAGAAATAGAGTTGATCGTC | |
| | AAAACCGACATTGCGACCGACGGTGGC | |
| | GATAGGCATCCGGGTGGTGCTCAAAAG | |
| | CAGCTTCGCCTGACTGATGCGCTGGTC | |
| | CTCGCGCCAGCTTAATACGCTAATCCC | |
| | TAACTGCTGGCGGAACAAATGCGACAG | |
| | ACGCGACGGCGACAGGCAGACATGCTG | |
| | TGCGACGCTGGCGATATCAAAATTACT | |
| | GTCTGCCAGGTGATCGCTGATGTACTG | |
| | ACAAGCCTCGCGTACCCGATTATCCAT | |
| | CGGTGGATGGAGCGACTCGTTAATCGC | |
| | TTCCATGCGCCGCAGTAACAATTGCTC | |
| | AAGCAGATTTATCGCCAGCAATTCCGA | |
| | ATAGCGCCCTTCCCCTTGTCCGGCATT | |
| | AATGATTTGCCCAAACAGGTCGCTGAA | |
| | ATGCGGCTGGTGCGCTTCATCCGGGCG | |
| | AAAGAAACCGGTATTGGCAAATATCGA | |
| | CGGCCAGTTAAGCCATTCATGCCAGTA | |
| | GGCGCGCGGACGAAAGTAAACCCACTG | |
| | GTGATACCATTCGTGAGCCTCCGGATG | |
| | ACGACCGTAGTGATGAATCTCTCCAGG | |
| | CGGGAACAGCAAAATATCACCCGGTCG | |
| | GCAGACAAATTCTCGTCCCTGATTTTT | |
| | CACCACCCCCTGACCGCGAATGGTGAG | |
| | ATTGAGAATATAACCTTTCATTCCCAG | |
| | CGGTCGGTCGATAAAAAAATCGAGATA | |
| | ACCGTTGGCCTCAATCGGCGTTAAACC | |
| | CGCCACCAGATGGGCGTTAAACGAGTA | |
| | TCCCGGCAGCAGGGGATCATTTTGCGC | |
| | TTCAGCCATACTTTTCATACTCCCGCC | |
| | ATTCAGAGAAGAAACCAATTGTCCATA | |
| | TTGCAT | |
| AraC | MQYGQLVSSLNGGSMKSMAEAQNDPLL | 105 |
| polypeptide | PGYSFNAHLVAGLTPIEANGYLDFFID | |
| | RPLGMKGYILNLTIRGQGVVKNQGREF | |
| | VCRPGDILLFPPGEIHHYGRHPEAHEW | |
| | YHQWVYFRPRAYWHEWLNWPSIFANTG | |
| | FFRPDEAHQPHFSDLFGQIINAGQGEG | |
| | RYSELLAINLLEQLLLRRMEAINESLH | |
| | PPMDNRVREACQYISDHLADSNFDIAS | |
| | VAQHVCLSPSRLSHLFRQQLGISVLSW | |
| | REDQRISQAKLLLSTTRMPIATVGRNV | |
| | GFDDQLYFSRVFKKCTGASPSEFRAGC | |
| | E* | |
| Region | CGGTGAGCATCACATCACCACAATTCA | 106 |
| comprising | GCAAATTGTGAACATCATCACGTTCAT | |
| rhamnose | CTTTCCCTGGTTGCCAATGGCCCATTT | |
| inducible | TCCTGTCAGTAACGAGAAGGTCGCGAA | |
| promoter | TCAGGCGCTTTTTAGACTGGTCGTAAT | |
| | GAAATTCAGCTGTCACCGGATGTGCTT | |
| | TCCGGTCTGATGAGTCCGTGAGGACGA | |
| | AACAGCCTCTACAAATAATTTTGTTTA | |
| | AAACAACACCCACTAAGATAACTCTAG | |
| | AAATAATTTTGTTTAACTTTAAGAAGG | |
| | AGATATACAT | |
| Lac | ATTCACCACCCTGAATTGACTCTCTTC | 107 |
| Promoter | CGGGCGCTATCATGCCATACCGCGAAA | |
| region | GGTTTTGCGCCATTCGATGGCGCGCCG | |
| | CTTCGTCAGGCCACATAGCTTTCTTGT | |
| | TCTGATCGGAACGATCGTTGGCTGTGT | |
| | TGACAATTAATCATCGGCTCGTATAAT | |
| | GTGTGGAATTGTGAGCGCTCACAATTA | |
| | GCTGTCACCGGATGTGCTTTCCGGTCT | |
| | GATGAGTCCGTGAGGACGAAACAGCCT | |
| | CTACAAATAATTTTGTTTAAAACAACA | |
| | CCCACTAAGATAACTCTAGAAATAATT | |
| | TTGTTTAACTTTAAGAAGGAGATATAC | |
| | AT | |
| LacO | GGAATTGTGAGCGCTCACAATT | 108 |
| LacI (in | TCACTGCCCGCTTTCCAGTCGGGAAAC | 109 |
| reverse | CTGTCGTGCCAGCTGCATTAATGAATC | |
| orientation) | GGCCAACGCGCGGGGAGAGGCGGTTTG | |
| | CGTATTGGGCGCCAGGGTGGTTTTTCT | |
| | TTTCACCAGTGAGACTGGCAACAGCTG | |
| | ATTGCCCTTCACCGCCTGGCCCTGAGA | |
| | GAGTTGCAGCAAGCGGTCCACGCTGGT | |
| | TTGCCCCAGCAGGCGAAAATCCTGTTT | |
| | GATGGTGGTTAACGGCGGGATATAACA | |
| | TGAGCTATCTTCGGTATCGTCGTATCC | |
| | CACTACCGAGATATCCGCACCAACGCG | |
| | CAGCCCGGACTCGGTAATGGCGCGCAT | |
| | TGCGCCCAGCGCCATCTGATCGTTGGC | |
| | AACCAGCATCGCAGTGGGAACGATGCC | |
| | CTCATTCAGCATTTGCATGGTTTGTTG | |
| | AAAACCGGACATGGCACTCCAGTCGCC | |
| | TTCCCGTTCCGCTATCGGCTGAATTTG | |
| | ATTGCGAGTGAGATATTTATGCCAGCC | |
| | AGCCAGACGCAGACGCGCCGAGACAGA | |
| | ACTTAATGGGCCCGCTAACAGCGCGAT | |
| | TTGCTGGTGACCCAATGCGACCAGATG | |
| | CTCCACGCCCAGTCGCGTACCGTCCTC | |
| | ATGGGAGAAAATAATACTGTTGATGGG | |
| | TGTCTGGTCAGAGACATCAAGAAATAA | |
| | CGCCGGAACATTAGTGCAGGCAGCTTC | |
| | CACAGCAATGGCATCCTGGTCATCCAG | |
| | CGGATAGTTAATGATCAGCCCACTGAC | |
| | GCGTTGCGCGAGAAGATTGTGCACCGC | |
| | CGCTTTACAGGCTTCGACGCCGCTTCG | |
| | TTCTACCATCGACACCACCACGCTGGC | |
| | ACCCAGTTGATCGGCGCGAGATTTAAT | |
| | CGCCGCGACAATTTGCGACGGCGCGTG | |
| | CAGGGCCAGACTGGAGGTGGCAACGCC | |
| | AATCAGCAACGACTGTTTGCCCGCCAG | |
| | TTGTTGTGCCACGCGGTTGGGAATGTA | |
| | ATTCAGCTCCGCCATCGCCGCTTCCAC | |
| | TTTTTCCCGCGTTTTCGCAGAAACGTG | |
| | GCTGGCCTGGTTCACCACGCGGGAAAC | |
| | GGTCTGATAAGAGACACCGGCATACTC | |
| | TGCGACATCGTATAACGTTACTGGTTT | |
| | CAT | |
| LacI | MKPVTLYDVAEYAGVSYQTVSRVVNQA | 110 |
| polypeptide | SHVSAKTREKVEAAMAELNYIPNRVAQ | |
| sequence | QLAGKQSLLIGVATSSLALHAPSQIVA | |
| | AIKSRADQLGASVVVSMVERSGVEACK | |
| | AAVHNLLAQRVSGLIINYPLDDQDAIA | |
| | VEAACTNVPALFLDVSDQTPINSIIFS | |
| | HEDGTRLGVEHLVALGHQQIALLAGPL | |
| | SSVSARLRLAGWHKYLTRNQIQPIAER | |
| | EGDWSAMSGFQQTMQMLNEGIVPTAML | |
| | VANDQMALGAMRAITESGLRVGADISV | |
| | VGYDDTEDSSCYIPPLTTIKQDFRLLG | |
| | QTSVDRLLQLSQGQAVKGNQLLPVSLV | |
| | KRKTTLAPNTQTASPRALADSLMQLAR | |
| | QVSRLESGQ | |
| TetR-tet | Ttaagacccactttcacatttaagttg | 111 |
| promoter | tttttctaatccgcatatgatcaattc | |
| construct | aaggccgaataagaaggctggctctgc | |
| | accttggtgatcaaataattcgatagc | |
| | ttgtcgtaataatggcggcatactatc | |
| | agtagtaggtgatccctttcttattag | |
| | cgacttgatgctcttgatatccaatac | |
| | gcaacctaaagtaaaatgccccacagc | |
| | gctgagtgcatataatgcattctctag | |
| | tgaaaaaccttgttggcataaaaaggc | |
| | taattgattttcgagagtttcatactg | |
| | tttttctgtaggccgtgtacctaaatg | |
| | tacttttgctccatcgcgatgacttag | |
| | taaagcacatctaaaacttttagcgtt | |
| | attacgtaaaaaatcttgccagctttc | |
| | cccttctaaagggcaaaagtgagtatg | |
| | gtgcctatctaacatctcaatggctaa | |
| | ggcgtcgagcaaagcccgcttattttt | |
| | tacatgccaatacaatgtaggctgctc | |
| | tacacctagcttctgggcgagtttacg | |
| | ggttgttaaaccttcgattccgacctc | |
| | attaagcagctctaatgcgctgttaat | |
| | cactttacttttatctaatctagacat | |
| | cattaattcctaatttttgttgacact | |
| | ctatcattgatagagttattttaccac | |
| | tccctatcagtgatagagaaaagtgaa | |
| | ctctagaaataattttgtttaacttta | |
| | agaaggagatatacat | |
| Region | ACGTTAAATCTATCACCGCAAGGGATA | 112 |
| comprising | AATATCTAACACCGTGCGTGTTGACTA | |
| Temperature | TTTTACCTCTGGCGGTGATAATGGTTG | |
| sensitive | CATAGCTGTCACCGGATGTGCTTTCCG | |
| promoter | GTCTGATGAGTCCGTGAGGACGAAACA | |
| | GCCTCTACAAATAATTTTGTTTAAAAC | |
| | AACACCCACTAAGATAACTCTAGAAAT | |
| | AATTTTGTTTAACTTTAAGAAGGAGAT | |
| | ATACAT | |
| mutant | TCAGCCAAACGTCTCTTCAGGCCACTG | 113 |
| cI857 | ACTAGCGATAACTTTCCCCACAACGGA | |
| repressor | ACAACTCTCATTGCATGGGATCATTGG | |
| | GTACTGTGGGTTTAGTGGTTGTAAAAA | |
| | CACCTGACCGCTATCCCTGATCAGTTT | |
| | CTTGAAGGTAAACTCATCACCCCCAAG | |
| | TCTGGCTATGCAGAAATCACCTGGCTC | |
| | AACAGCCTGCTCAGGGTCAACGAGAAT | |
| | TAACATTCCGTCAGGAAAGCTTGGCTT | |
| | GGAGCCTGTTGGTGCGGTCATGGAATT | |
| | ACCTTCAACCTCAAGCCAGAATGCAGA | |
| | ATCACTGGCTTTTTTGGTTGTGCTTAC | |
| | CCATCTCTCCGCATCACCTTTGGTAAA | |
| | GGTTCTAAGCTTAGGTGAGAACATCCC | |
| | TGCCTGAACATGAGAAAAAACAGGGTA | |
| | CTCATACTCACTTCTAAGTGACGGCTG | |
| | CATACTAACCGCTTCATACATCTCGTA | |
| | GATTTCTCTGGCGATTGAAGGGCTAAA | |
| | TTCTTCAACGCTAACTTTGAGAATTTT | |
| | TGTAAGCAATGCGGCGTTATAAGCATT | |
| | TAATGCATTGATGCCATTAAATAAAGC | |
| | ACCAACGCCTGACTGCCCCATCCCCAT | |
| | CTTGTCTGCGACAGATTCCTGGGATAA | |
| | GCCAAGTTCATTTTTCTTTTTTTCATA | |
| | AATTGCTTTAAGGCGACGTGCGTCCTC | |
| | AAGCTGCTCTTGTGTTAATGGTTTCTT | |
| | TTTTGTGCTCAT | |
| RBS and | CTCTAGAAATAATTTTGTTTAACTTTA | 114 |
| leader | AGAAGGAGATATACAT | |
| region | | |
| mutant | MSTKKKPLTQEQLEDARRLKAIYEKKK | 115 |
| cI857 | NELGLSQESVADKMGMGQSGVGALFNG | |
| repressor | INALNAYNAALLTKILKVSVEEFSPSI | |
| polypeptide | AREIYEMYEAVSMQPSLRSEYEYPVFS | |
| sequence | HVQAGMFSPKLRTFTKGDAERWVSTTK | |
| | KASDSAFWLEVEGNSMTAPTGSKPSFP | |
| | DGMLILVDPEQAVEPGDFCIARLGGDE | |
| | FTFKKLIRDSGQVFLQPLNPQYPMIPC | |
| | NESCSVVGKVIASQWPEETFG | |
| PssB | TCACCTTTCCCGGATTAAACGCTTTTT | 116 |
| promoter | TGCCCGGTGGCATGGTGCTACCGGCGA | |
| | TCACAAACGGTTAATTATGACACAAAT | |
| | TGACCTGAATGAATATACAGTATTGGA | |
| | ATGCATTACCCGGAGTGTTGTGTAACA | |
| | ATGTCTGGCCAGGTTTGTTTCCCGGAA | |
| | CCGAGGTCACAACATAGTAAAAGCGCT | |
| | ATTGGTAATGGTACAATCGCGCGTTTA | |
| | CACTTATTC | |

| Description | |
|---|---|
| and SEQ ID | |
| NO | Sequence |
| FNR | AGTTGTTCTTATTGGTGGTGTTGCTTT |
| promoter with | ATGGTTGCATCGTAGTAAATGGTTGTA |
| RBS and | ACAAAAGCAATTTTTCCGGCTGTCTGT |
| leader region | ATACAAAAACGCCGCAAAGTTTGAGCG |
| (underlined), | AAGTCAATAAACTCTCTACCCATTCAG |
| FNR binding | GGCAATATCTCTCTTggatccaaagtg |
| site bold | aactctagaaataattttgtttaactt |
| SEQ ID NO: | taagaaggagatatacat |
| 117 | |
| FNR binding | TTGAGCGAAGTCAA |
| site | |
| SEQ ID NO: | |
| 118 | |
| FNR | AGTTGTTCTTATTGGTGGTGTTGCTTT |
| promoter | ATGGTTGCATCGTAGTAAATGGTTGTA |
| without RBS | ACAAAAGCAATTTTTCCGGCTGTCTGT |
| and leader | ATACAAAAACGCCGCAAAGTTTGAGCG |
| region | AAGTCAATAAACTCTCTACCCATTCAG |
| SEQ ID NO: | GGCAATATCTCTCTTggatccaaagtg |
| 119 | aa |
| RBS and | ctctagaaataattttgtttaacttta |
| leader region | agaaggagatatacat |
| SEQ ID NO: | |
| 120 | |
| LeuDH-kivD- | ATGACTCTTGAAATCTTTGAATATTTA |
| adh2-brnQ | GAAAAGTACGACTACGAGCAGGTTGTA |
| construct | TTTTGTCAAGACAAGGAGTCTGGGCTG |
| SEQ ID NO: | AAGGCCATCATTGCCATCCACGACACA |
| 121 | ACCTTAGGCCCGGCGCTTGGCGGAACC |
| | CGCATGTGGACCTACGACTCCGAGGAG |
| | GCGGCCATCGAGGACGCACTTCGTCTT |
| | GCTAAGGGTATGACCTATAAGAACGCG |
| | GCAGCCGGTCTGAATCTGGGGGGTGCT |
| | AAGACTGTAATCATCGGTGATCCACGC |
| | AAGGATAAGAGTGAAGCAATGTTTCGC |
| | GCTTTAGGGCGCTATATTCAGGGCTTG |
| | AACGGCCGCTACATTACCGCAGAAGAC |
| | GTAGGGACAACAGTAGACGACATGGAC |
| | ATCATCCATGAGGAAACTGATTTCGTG |
| | ACCGGTATTTCACCTTCATTCGGGTCA |
| | TCCGGTAACCCTTCCCCCGTAACAGCC |
| | TATGGGGTTTATCGCGGAATGAAGGCC |
| | GCAGCCAAGGAGGCATTTGGCACTGAC |
| | AATTTAGAAGGAAAAGTAATTGCTGTC |
| | CAAGGCGTGGGCAATGTGGCCTACCAT |
| | TTGTGTAAACACCTTCACGCGGAAGGT |
| | GCAAAATTGATCGTTACGGATATTAAC |
| | AAGGAGGCAGTCCAGCGCGCTGTAGAG |
| | GAATTTGGAGCATCGGCTGTGGAACCA |
| | AATGAGATCTACGGTGTAGAATGTGAC |
| | ATTTACGCTCCATGCGCACTTGGTGCC |
| | ACGGTGAATGACGAGACCATCCCCCAA |
| | CTTAAGGCGAAGGTAATCGCTGGTTCA |
| | GCTAACAACCAATTAAAAGAGGACCGT |
| | CACGGAGATATCATCCACGAAATGGGT |
| | ATCGTGTACGCCCCCGATTATGTTATC |
| | AACGCGGGCGGCGTAATCAACGTAGCC |
| | GATGAGCTTTATGGATACAACCGCGAA |
| | CGTGCGCTGAAACGCGTGGAAAGCATT |
| | TATGACACGATCGCAAAGGTAATCGAG |
| | ATCAGTAAGCGCGACGGCATTGCGACA |
| | TACGTGGCAGCGGACCGTCTGGCCGAA |
| | GAACGCATCGCGAGTTTGAAGAATAGC |
| | CGTAGTACCTACTTGCGCAACGGGCAC |
| | GATATTATCAGCCGTCGCtgataagaa |
| | ggagatatacatatgtatacagtagga |
| | GATTACTTATTGGACCGGTTGCACGAA |
| | CTTGGAATTGAGGAAATTTTTGGAGTT |
| | CCGGGTGACTACAACCTGCAGTTCCTT |
| | GACCAAATCATCTCCCATAAGGACATG |
| | AAATGGGTCGGCAATGCCAATGAGCTG |
| | AACGCATCATATATGGCAGACGGGTAT |
| | GCTCGGACCAAAAAGGCTGCAGCATTC |
| | CTTACCACGTTTGGCGTGGGGGAATTA |
| | AGTGCTGTAAATGGACTGGCAGGATCC |
| | TATGCGGAGAATTTACCGGTAGTCGAA |
| | ATTGTTGGCTCGCCTACGTCCAAGGTG |
| | CAGAATGAGGGGAAATTCGTCCATCAC |
| | ACACTTGCAGACGGTGATTTTAAGCAC |
| | TTTATGAAGATGCATGAGCCGGTAACG |
| | GCTGCGCGGACGCTTCTTACTGCGGAA |
| | AACGCAACAGTAGAGATTGATCGCGTT |
| | CTGAGCGCACTGCTTAAGGAACGGAAG |
| | CCCGTCTATATTAACTTACCGGTAGAC |
| | GTGGCCGCAGCCAAAGCCGAAAAACCA |
| | AGCCTGCCTCTTAAGAAGGAGAATTCC |
| | ACGTCCAACACCAGTGACCAAGAGATT |
| | TTGAACAAAATTCAAGAGTCTTTGAAG |
| | AACGCGAAGAAGCCCATCGTAATTACA |
| | GGACATGAGATTATCTCGTTTGGCCTG |
| | GAGAAAACGGTTACACAGTTTATTTCC |
| | AAAACGAAGTTACCTATAACGACGTTA |
| | AACTTTGGAAAGAGCTCTGTGGATGAG |
| | GCACTTCCTAGTTTCTTAGGAATCTAT |
| | AATGGGACCCTTTCAGAGCCAAACTTA |
| | AAGGAATTCGTTGAAAGTGCGGATTTT |
| | ATCTTAATGCTTGGGGTTAAATTGACT |
| | GATTCCAGCACCGGAGCTTTTACGCAC |
| | CATTTAAACGAGAACAAAATGATCTCT |
| | TTGAATATCGACGAAGGCAAAATTTTT |
| | AATGAAAGAATTCAGAACTTTGATTTT |
| | GAATCCCTTATTAGTTCACTTTTAGAT |
| | TTAAGTGAAATAGAGTATAAGGGAAAG |
| | TATATAGACAAGAAGCAAGAGGATTTC |
| | GTTCCGTCTAATGCTCTTTTAAGTCAA |
| | GACAGACTTTGGCAGGCGGTTGAGAAC |
| | CTTACACAATCCAATGAAACGATAGTC |
| | GCCGAACAAGGGACCAGTTTCTTCGGC |
| | GCTTCTTCCATATTCCTGAAGTCTAAG |
| | TCTCATTTCATTGGACAGCCCCTGTGG |
| | GGGTCTATAGGATATACGTTTCCCGCA |
| | GCTCTTGGAAGCCAGATCGCCGATAAG |
| | GAGAGCAGACACCTGTTGTTCATCGGG |
| | GACGGCTCGTTGCAGCTGACTGTTCAG |
| | GAACTGGGGTTGGCGATCAGAGAGAAG |
| | ATTAATCCCATTTGCTTTATCATAAAT |
| | AATGATGGTTATACCGTAGAACGTGAG |
| | ATTCATGGACCTAATCAGAGCTATAAT |
| | GACATTCCTATGTGGAACTATTCAAAA |
| | TTGCCAGAGAGTTTTGGTGCAACTGAG |
| | GATCGCGTTGTTAGTAAAATAGTCCGC |
| | ACGGAGAACGAGTTTGTCAGCGTAATG |
| | AAGGAGGCCCAAGCGGACCCTAATCGG |
| | ATGTACTGGATCGAACTTATTCTGGCT |
| | AAAGAAGGAGCACCTAAAGTTTTAAAG |
| | AAGATGGGAAAACTTTTTgctgaacaa |
| | aataaatcataataagaaggagatata |
| | catatgtctattccaGAAACGCAGAAA |
| | GCCATCATATTTTATGAATCGAACGGA |
| | AAACTTGAGCACAAGGACATCCCCGTC |
| | CCGAAGCCAAAACCTAATGAGTTGCTT |
| | ATCAACGTTAAGTATTCGGGCGTATGC |
| | CACACAGACTTGCACGCATGGCACGGG |
| | GATTGGCCCTTACCGACTAAGTTGCCG |
| | TTAGTGGGCGGACATGAGGGGGCGGGA |
| | GTCGTAGTGGGAATGGGAGAGAACGTG |
| | AAGGGTTGGAAGATTGGAGATTATGCT |
| | GGGATTAAGTGGTTGAATGGGAGCTGC |
| | ATGGCCTGCGAATATTGTGAACTTGGA |
| | AATGAGAGCAATTGCCCACATGCTGAC |
| | TTGTCCGGTTACACACATGACGGTTCA |
| | TTCCAGGAATATGCTACGGCTGATGCA |
| | GTCCAAGCAGCGCATATCCCGCAAGGG |
| | ACGGACTTAGCAGAAGTAGCGCCCATT |
| | CTTTGCGCTGGGATCACCGTATATAAA |
| | GCGTTAAAGAGCGCAAATTTACGGGCC |
| | GGACATTGGGCGGCGATCAGCGGGGCC |
| | GCAGGGGGGCTGGGCAGCTTGGCCGTC |
| | CAGTACGCTAAAGCTATGGGTTATCGG |
| | GTTTTGGGCATTGACGGAGGACCGGGA |
| | AAGGAGGAATTATTCACGTCCTTGGGA |
| | GGAGAGGTATTCATTGACTTTACCAAG |
| | GAAAAAGATATCGTCTCTGCTGTAGTA |
| | AAGGCTACCAATGGCGGTGCCCACGGA |
| | ATCATAAATGTTTCAGTTTCTGAAGCG |
| | GCGATCGAAGCGTCCACTAGATATTGC |
| | CGTGCAAATGGGACAGTCGTACTTGTA |
| | GGACTTCCGGCTGGCGCCAAATGCAGC |
| | TCCGATGTATTTAATCATGTCGTGAAG |
| | TCAATCTCTATCGTTGGTTCATATGTA |
| | GGAAACCGCGCCGATACTCGTGAGGCT |
| | CTTGACTTTTTTGCCAGAGGCCTGGTT |
| | AAGTCCCCCATAAAAGTTGTTGGCTTA |
| | TCCAGCTTACCCGAAATATACGAGAAG |
| | ATGGAGAAGGGCCAGATCGCGGGGAGA |
| | tacgttgagacacttctaaataataag |
| | aaggagatatacatatgacccatcaat |
| | taagatcgcgcgatatcatcgctctgg |
| | gattatgacatttgcgttgacgtcggc |
| | gcaggtaacattattaccctccaatgg |
| | tcggcttgcaggcaggcgaacacgtct |
| | ggactgcggcattcggcacctcattac |
| | tgccgaggcctaccggtattaacggta |
| | gtggcgctggcaaaagaggcggcggtg |
| | agacagtctcagcacgccaattggtaa |
| | agtcgctggcgtactgctggcaacagt |
| | ttgttacctggcggtggggccgctttt |
| | tgctacgccgcgtacagctaccgatca |
| | ttgaagtgggcattgcgccgctgacgg |
| | gtgattccgcgctgccgctgatattta |
| | cagcctggtctatttcgctatcgttat |
| | tctggatcgctctatccgggcaagctg |
| | ctggataccgtgggcaacttccagcgc |
| | cgctgaaaattatcgcgctggtcatcc |
| | tgtctgagccgcaattatctggccggc |
| | gggactatcagtacggcgactgaggct |
| | tatcaaaacgctgcgttactaacggct |
| | tcgtcaacggctatctgaccatggata |
| | cgctgggcgcaatggtgtaggtatcgt |
| | tattgttaacgcggcgcgactcgtggc |
| | gttaccgaagcgcgtctgctgacccgt |
| | tataccgtctgggctggcctgatggcg |
| | ggtgttggtctgactctgctgtacctg |
| | gcgctgttccgtctgggttcagacagc |
| | gcgtcgctggtcgatcagtctgcaaac |
| | ggtgcggcgatcctgcatgcttacgtt |
| | cagcatacctaggcggcggcggtagat |
| | cctgctggcggcgttaatcttcatcgc |
| | ctgcctggtcacggcggaggcctgacc |
| | tgtgcttgtgcagaattatcgcccagt |
| | acgtaccgctctcttatcgtacgctgg |
| | tgatatcctcggcggcactcgatggtg |
| | gtgtctaacctcggcttgagccagctg |
| | attcagatctctgtaccggtgctgacc |
| | gccatttatccgccgtgtatcgcactg |
| | gagtattaagattacacgctcatggtg |
| | gcataattcgtcccgcgtgattgctcc |
| | gccgatgatatcagcctgctattggta |
| | ttctcgacgggatcaaggcatctgcat |
| | tcagcgatatcttaccgtcctgggcgc |
| | agcgataccgctggccgaacaaggtct |
| | ggcgtggttaatgccaacagtggtgat |
| | ggtggactggccattatctgggatcgt |
| | gcggcaggtcgtcaggtgacctccagc |
| | gctcactaa |
| Pfnrs-LeuDH- | AGTTGTTCTTATTGGTGGTGTTGCTTT |
| kivD-adh2- | ATGGTTGCATCGTAGTAAATGGTTGTA |
| brnQ | ACAAAAGCAATTTTTCCGGCTGTCTGT |
| construct | ATACAAAAACGCCGCAAAGTTTGAGCG |
| (with | AAGTCAATAAACTCTCTACCCATTCAG |
| terminator) | GGCAATATCTCTCTTggatccaaagtg |
| (RBS are | aactctagaaataattttgtttaactt |
| underlined) | taagaaggagatatacatATGACTCTT |
| SEQ ID NO: | GAAATCTTTGAATATTTAGAAAAGTAC |
| 122 | GACTACGAGCAGGTTGTATTTTGTCAA |
| | GACAAGGAGTCTGGGCTGAAGGCCATC |
| | ATTGCCATCCACGACACAACCTTAGGC |
| | CCGGCGCTTGGCGGAACCCGCATGTGG |
| | ACCTACGACTCCGAGGAGGCGGCCATC |
| | GAGGACGCACTTCGTCTTGCTAAGGGT |
| | ATGACCTATAAGAACGCGGCAGCCGGT |
| | CTGAATCTGGGGGGTGCTAAGACTGTA |
| | ATCATCGGTGATCCACGCAAGGATAAG |
| | AGTGAAGCAATGTTTCGCGCTTTAGGG |
| | CGCTATATTCAGGGCTTGAACGGCCGC |
| | TACATTACCGCAGAAGACGTAGGGACA |
| | ACAGTAGACGACATGGACATCATCCAT |
| | GAGGAAACTGATTTCGTGACCGGTATT |
| | TCACCTTCATTCGGGTCATCCGGTAAC |
| | CCTTCCCCCGTAACAGCCTATGGGGTT |
| | TATCGCGGAATGAAGGCCGCAGCCAAG |
| | GAGGCATTTGGCACTGACAATTTAGAA |
| | GGAAAAGTAATTGCTGTCCAAGGCGTG |
| | GGCAATGTGGCCTACCATTTGTGTAAA |
| | CACCTTCACGCGGAAGGTGCAAAATTG |
| | ATCGTTACGGATATTAACAAGGAGGCA |
| | GTCCAGCGCGCTGTAGAGGAATTTGGA |
| | GCATCGGCTGTGGAACCAAATGAGATC |
| | TACGGTGTAGAATGTGACATTTACGCT |
| | CCATGCGCACTTGGTGCCACGGTGAAT |
| | GACGAGACCATCCCCCAACTTAAGGCG |
| | AAGGTAATCGCTGGTTCAGCTAACAAC |
| | CAATTAAAAGAGGACCGTCACGGAGAT |
| | ATCATCCACGAAATGGGTATCGTGTAC |
| | GCCCCCGATTATGTTATCAACGCGGGC |
| | GGCGTAATCAACGTAGCCGATGAGCTT |
| | TATGGATACAACCGCGAACGTGCGCTG |
| | AAACGCGTGGAAAGCATTTATGACACG |
| | ATCGCAAAGGTAATCGAGATCAGTAAG |
| | CGCGACGGCATTGCGACATACGTGGCA |
| | GCGGACCGTCTGGCCGAAGAACGCATC |
| | GCGAGTTTGAAGAATAGCCGTAGTACC |
| | TACTTGCGCAACGGGCACGATATTATC |
| | AGCCGTCGCtgataagaaggagatata |
| | catatgtatacagtaggaGATTACTTA |
| | TTGGACCGGTTGCACGAACTTGGAATT |
| | GAGGAAATTTTTGGAGTTCCGGGTGAC |
| | TACAACCTGCAGTTCCTTGACCAAATC |
| | ATCTCCCATAAGGACATGAAATGGGTC |
| | GGCAATGCCAATGAGCTGAACGCATCA |
| | TATATGGCAGACGGGTATGCTCGGACC |
| | AAAAAGGCTGCAGCATTCCTTACCACG |
| | TTTGGCGTGGGGGAATTAAGTGCTGTA |
| | AATGGACTGGCAGGATCCTATGCGGAG |
| | AATTTACCGGTAGTCGAAATTGTTGGC |
| | TCGCCTACGTCCAAGGTGCAGAATGAG |
| | GGGAAATTCGTCCATCACACACTTGCA |
| | GACGGTGATTTTAAGCACTTTATGAAG |
| | ATGCATGAGCCGGTAACGGCTGCGCGG |
| | ACGCTTCTTACTGCGGAAAACGCAACA |
| | GTAGAGATTGATCGCGTTCTGAGCGCA |
| | CTGCTTAAGGAACGGAAGCCCGTCTAT |
| | ATTAACTTACCGGTAGACGTGGCCGCA |
| | GCCAAAGCCGAAAAACCAAGCCTGCCT |
| | CTTAAGAAGGAGAATTCCACGTCCAAC |
| | ACCAGTGACCAAGAGATTTTGAACAAA |
| | ATTCAAGAGTCTTTGAAGAACGCGAAG |
| | AAGCCCATCGTAATTACAGGACATGAG |
| | ATTATCTCGTTTGGCCTGGAGAAAACG |
| | GTTACACAGTTTATTTCCAAAACGAAG |
| | TTACCTATAACGACGTTAAACTTTGGA |
| | AAGAGCTCTGTGGATGAGGCACTTCCT |
| | AGTTTCTTAGGAATCTATAATGGGACC |
| | CTTTCAGAGCCAAACTTAAAGGAATTC |
| | GTTGAAAGTGCGGATTTTATCTTAATG |
| | CTTGGGGTTAAATTGACTGATTCCAGC |
| | ACCGGAGCTTTTACGCACCATTTAAAC |
| | GAGAACAAAATGATCTCTTTGAATATC |
| | GACGAAGGCAAAATTTTTAATGAAAGA |
| | ATTCAGAACTTTGATTTTGAATCCCTT |
| | ATTAGTTCACTTTTAGATTTAAGTGAA |
| | ATAGAGTATAAGGGAAAGTATATAGAC |
| | AAGAAGCAAGAGGATTTCGTTCCGTCT |
| | AATGCTCTTTTAAGTCAAGACAGACTT |
| | TGGCAGGCGGTTGAGAACCTTACACAA |
| | TCCAATGAAACGATAGTCGCCGAACAA |
| | GGGACCAGTTTCTTCGGCGCTTCTTCC |
| | ATATTCCTGAAGTCTAAGTCTCATTTC |
| | ATTGGACAGCCCCTGTGGGGGTCTATA |
| | GGATATACGTTTCCCGCAGCTCTTGGA |
| | AGCCAGATCGCCGATAAGGAGAGCAGA |
| | CACCTGTTGTTCATCGGGGACGGCTCG |
| | TTGCAGCTGACTGTTCAGGAACTGGGG |
| | TTGGCGATCAGAGAGAAGATTAATCCC |
| | ATTTGCTTTATCATAAATAATGATGGT |
| | TATACCGTAGAACGTGAGATTCATGGA |
| | CCTAATCAGAGCTATAATGACATTCCT |
| | ATGTGGAACTATTCAAAATTGCCAGAG |
| | AGTTTTGGTGCAACTGAGGATCGCGTT |
| | GTTAGTAAAATAGTCCGCACGGAGAAC |
| | GAGTTTGTCAGCGTAATGAAGGAGGCC |
| | CAAGCGGACCCTAATCGGATGTACTGG |
| | ATCGAACTTATTCTGGCTAAAGAAGGA |
| | GCACCTAAAGTTTTAAAGAAGATGGGA |
| | AAACTTTTTgctgaacaaaataaatca |
| | taataagaaggagatatacatatgtct |
| | attccaGAAACGCAGAAAGCCATCATA |
| | TTTTATGAATCGAACGGAAAACTTGAG |
| | CACAAGGACATCCCCGTCCCGAAGCCA |
| | AAACCTAATGAGTTGCTTATCAACGTT |
| | AAGTATTCGGGCGTATGCCACACAGAC |
| | TTGCACGCATGGCACGGGGATTGGCCC |
| | TTACCGACTAAGTTGCCGTTAGTGGGC |
| | GGACATGAGGGGGCGGGAGTCGTAGTG |
| | GGAATGGGAGAGAACGTGAAGGGTTGG |
| | AAGATTGGAGATTATGCTGGGATTAAG |
| | TGGTTGAATGGGAGCTGCATGGCCTGC |
| | GAATATTGTGAACTTGGAAATGAGAGC |
| | AATTGCCCACATGCTGACTTGTCCGGT |
| | TACACACATGACGGTTCATTCCAGGAA |
| | TATGCTACGGCTGATGCAGTCCAAGCA |
| | GCGCATATCCCGCAAGGGACGGACTTA |
| | GCAGAAGTAGCGCCCATTCTTTGCGCT |
| | GGGATCACCGTATATAAAGCGTTAAAG |
| | AGCGCAAATTTACGGGCCGGACATTGG |
| | GCGGCGATCAGCGGGGCCGCAGGGGGG |
| | CTGGGCAGCTTGGCCGTCCAGTACGCT |
| | AAAGCTATGGGTTATCGGGTTTTGGGC |
| | ATTGACGGAGGACCGGGAAAGGAGGAA |
| | TTATTCACGTCCTTGGGAGGAGAGGTA |
| | TTCATTGACTTTACCAAGGAAAAAGAT |
| | ATCGTCTCTGCTGTAGTAAAGGCTACC |
| | AATGGCGGTGCCCACGGAATCATAAAT |
| | GTTTCAGTTTCTGAAGCGGCGATCGAA |
| | GCGTCCACTAGATATTGCCGTGCAAAT |
| | GGGACAGTCGTACTTGTAGGACTTCCG |
| | GCTGGCGCCAAATGCAGCTCCGATGTA |
| | TTTAATCATGTCGTGAAGTCAATCTCT |
| | ATCGTTGGTTCATATGTAGGAAACCGC |
| | GCCGATACTCGTGAGGCTCTTGACTTT |
| | TTTGCCAGAGGCCTGGTTAAGTCCCCC |
| | ATAAAAGTTGTTGGCTTATCCAGCTTA |
| | CCCGAAATATACGAGAAGATGGAGAAG |
| | GGCCAGATCGCGGGGAGAtacgttgag |
| | acacttctaaataataagaaggagata |
| | tacatatgacccatcaattaagatcgc |
| | gcgatatcatcgctctgggattatgac |
| | atttgcgttgacgtcggcgcaggtaac |
| | attattaccctccaatggtcggcttgc |
| | aggcaggcgaacacgtctggactgcgg |
| | cattcggcacctcattactgccgaggc |
| | ctaccggtattaacggtagtggcgctg |
| | gcaaaagaggcggcggtgagacagtct |
| | cagcacgccaattggtaaagtcgctgg |
| | cgtactgctggcaacagtttgttacct |
| | ggcggtggggccgctttttgctacgcc |
| | gcgtacagctaccgatcattgaagtgg |
| | gcattgcgccgctgacgggtgattccg |
| | cgctgccgctgatatttacagcctggt |
| | ctatttcgctatcgttattctggatcg |
| | ctctatccgggcaagctgctggatacc |
| | gtgggcaacttccagcgccgctgaaaa |
| | ttatcgcgctggtcatcctgtctgagc |
| | cgcaattatctggccggcgggactatc |
| | agtacggcgactgaggcttatcaaaac |
| | gctgcgttactaacggcttcgtcaacg |
| | gctatctgaccatggatacgctgggcg |
| | caatggtgtaggtatcgttattgttaa |
| | cgcggcgcgactcgtggcgttaccgaa |
| | gcgcgtctgctgacccgttataccgtc |
| | tgggctggcctgatggcgggtgttggt |
| | ctgactctgctgtacctggcgctgttc |
| | cgtctgggttcagacagcgcgtcgctg |
| | gtcgatcagtctgcaaacggtgcggcg |
| | atcctgcatgcttacgttcagcatacc |
| | taggcggcggcggtagatcctgctggc |
| | ggcgttaatcttcatcgcctgcctggt |
| | cacggcggaggcctgacctgtgcttgt |
| | gcagaattatcgcccagtacgtaccgc |
| | tctcttatcgtacgctggtgatatcct |
| | cggcggcactcgatggtggtgtctaac |
| | ctcggcttgagccagctgattcagatc |
| | tctgtaccggtgctgaccgccatttat |
| | ccgccgtgtatcgcactggagtattaa |
| | gattacacgctcatggtggcataattc |
| | gtcccgcgtgattgctccgccgatgat |
| | atcagcctgctattggtattctcgacg |
| | ggatcaaggcatctgcattcagcgata |
| | tcttaccgtcctgggcgcagcgatacc |
| | gctggccgaacaaggtctggcgtggtt |
| | aatgccaacagtggtgatggtggactg |
| | gccattatctgggatcgtgcggcaggt |
| | cgtcaggtgacctccagcgctcactaa |
| | tacgcatggcatggatgaCCGATGGTA |
| | GTGTGGGGTCTCCCCATGCGAGAGTAG |
| | GGAACTGCCAGGCATCAAATAAAACGA |
| | AAGGCTCAGTCGAAAGACTGGGCCTTT |
| | CGTTTTATCTGTTGTTTGTCGGTGAAC |
| | GCTCTCCTGAGTAGGACAAAT |
| Tet-LeuDH- | ttaagacccactacacatttaagttga |
| kivD-adh2- | tactaatccgcatatgatcaattcaag |
| brnQ | gccgaataagaaggctggctctgcacc |
| construct | aggtgatcaaataattcgatagatgtc |
| (tet | gtaataatggcggcatactatcagtag |
| Repressor | taggtgatccctttatattagcgactt |
| is in | gatgctcttgatcaccaatacgcaacc |
| reverse | taaagtaaaatgccccacagcgctgag |
| orientation | tgcatataatgcattctctagtgaaaa |
| and | accttgaggcataaaaaggctaattga |
| underlined; | ttacgagagatcatactgatactgtag |
| tet promoter | gccgtgtacctaaatgtacttttgctc |
| with RBS and | catcgcgatgacttagtaaagcacatc |
| leader region | taaaacttttagcgttattacgtaaaa |
| is in bold | aatcttgccagctttccccttctaaag |
| italics) | ggcaaaagtgagtatggtgcctatcta |
| SEQ ID NO: | acatctcaatggctaaggcgtcgagca |
| 123 | aagcccgcttattattacatgccaata |
| | caatgtaggctgctctacacctagatc |
| | tgggcgagtttacgggttgttaaacct |
| | tcgattccgacctcattaagcagctct |
| | aatgcgctgttaatcactttactttta |
| | tctaatctagacat |
| | [symbol] |
| | [symbol] |
| | [symbol] |
| | [symbol] |
| | [symbol] |
| | [symbol] |
| | ATGACTCTTGAAATCTTTGAATATTTA |
| | GAAAAGTACGACTACGAGCAGGTTGTA |
| | TTTTGTCAAGACAAGGAGTCTGGGCTG |
| | AAGGCCATCATTGCCATCCACGACACA |
| | ACCTTAGGCCCGGCGCTTGGCGGAACC |
| | CGCATGTGGACCTACGACTCCGAGGAG |
| | GCGGCCATCGAGGACGCACTTCGTCTT |
| | GCTAAGGGTATGACCTATAAGAACGCG |
| | GCAGCCGGTCTGAATCTGGGGGGTGCT |
| | AAGACTGTAATCATCGGTGATCCACGC |
| | AAGGATAAGAGTGAAGCAATGTTTCGC |
| | GCTTTAGGGCGCTATATTCAGGGCTTG |
| | AACGGCCGCTACATTACCGCAGAAGAC |
| | GTAGGGACAACAGTAGACGACATGGAC |
| | ATCATCCATGAGGAAACTGATTTCGTG |
| | ACCGGTATTTCACCTTCATTCGGGTCA |
| | TCCGGTAACCCTTCCCCCGTAACAGCC |
| | TATGGGGTTTATCGCGGAATGAAGGCC |
| | GCAGCCAAGGAGGCATTTGGCACTGAC |
| | AATTTAGAAGGAAAAGTAATTGCTGTC |
| | CAAGGCGTGGGCAATGTGGCCTACCAT |
| | TTGTGTAAACACCTTCACGCGGAAGGT |
| | GCAAAATTGATCGTTACGGATATTAAC |
| | AAGGAGGCAGTCCAGCGCGCTGTAGAG |
| | GAATTTGGAGCATCGGCTGTGGAACCA |
| | AATGAGATCTACGGTGTAGAATGTGAC |
| | ATTTACGCTCCATGCGCACTTGGTGCC |
| | ACGGTGAATGACGAGACCATCCCCCAA |
| | CTTAAGGCGAAGGTAATCGCTGGTTCA |
| | GCTAACAACCAATTAAAAGAGGACCGT |
| | CACGGAGATATCATCCACGAAATGGGT |
| | ATCGTGTACGCCCCCGATTATGTTATC |
| | AACGCGGGCGGCGTAATCAACGTAGCC |
| | GATGAGCTTTATGGATACAACCGCGAA |
| | CGTGCGCTGAAACGCGTGGAAAGCATT |
| | TATGACACGATCGCAAAGGTAATCGAG |
| | ATCAGTAAGCGCGACGGCATTGCGACA |
| | TACGTGGCAGCGGACCGTCTGGCCGAA |
| | GAACGCATCGCGAGTTTGAAGAATAGC |
| | CGTAGTACCTACTTGCGCAACGGGCAC |
| | GATATTATCAGCCGTCGCtgataagaa |
| | ggagatatacatatgtatacagtagga |
| | GATTACTTATTGGACCGGTTGCACGAA |
| | CTTGGAATTGAGGAAATTTTTGGAGTT |
| | CCGGGTGACTACAACCTGCAGTTCCTT |
| | GACCAAATCATCTCCCATAAGGACATG |
| | AAATGGGTCGGCAATGCCAATGAGCTG |
| | AACGCATCATATATGGCAGACGGGTAT |
| | GCTCGGACCAAAAAGGCTGCAGCATTC |
| | CTTACCACGTTTGGCGTGGGGGAATTA |
| | AGTGCTGTAAATGGACTGGCAGGATCC |
| | TATGCGGAGAATTTACCGGTAGTCGAA |
| | ATTGTTGGCTCGCCTACGTCCAAGGTG |
| | CAGAATGAGGGGAAATTCGTCCATCAC |
| | ACACTTGCAGACGGTGATTTTAAGCAC |
| | TTTATGAAGATGCATGAGCCGGTAACG |
| | GCTGCGCGGACGCTTCTTACTGCGGAA |
| | AACGCAACAGTAGAGATTGATCGCGTT |
| | CTGAGCGCACTGCTTAAGGAACGGAAG |
| | CCCGTCTATATTAACTTACCGGTAGAC |
| | GTGGCCGCAGCCAAAGCCGAAAAACCA |
| | AGCCTGCCTCTTAAGAAGGAGAATTCC |
| | ACGTCCAACACCAGTGACCAAGAGATT |
| | TTGAACAAAATTCAAGAGTCTTTGAAG |
| | AACGCGAAGAAGCCCATCGTAATTACA |
| | GGACATGAGATTATCTCGTTTGGCCTG |
| | GAGAAAACGGTTACACAGTTTATTTCC |
| | AAAACGAAGTTACCTATAACGACGTTA |
| | AACTTTGGAAAGAGCTCTGTGGATGAG |
| | GCACTTCCTAGTTTCTTAGGAATCTAT |
| | AATGGGACCCTTTCAGAGCCAAACTTA |
| | AAGGAATTCGTTGAAAGTGCGGATTTT |
| | ATCTTAATGCTTGGGGTTAAATTGACT |
| | GATTCCAGCACCGGAGCTTTTACGCAC |
| | CATTTAAACGAGAACAAAATGATCTCT |
| | TTGAATATCGACGAAGGCAAAATTTTT |
| | AATGAAAGAATTCAGAACTTTGATTTT |
| | GAATCCCTTATTAGTTCACTTTTAGAT |
| | TTAAGTGAAATAGAGTATAAGGGAAAG |
| | TATATAGACAAGAAGCAAGAGGATTTC |
| | GTTCCGTCTAATGCTCTTTTAAGTCAA |
| | GACAGACTTTGGCAGGCGGTTGAGAAC |
| | CTTACACAATCCAATGAAACGATAGTC |
| | GCCGAACAAGGGACCAGTTTCTTCGGC |
| | GCTTCTTCCATATTCCTGAAGTCTAAG |
| | TCTCATTTCATTGGACAGCCCCTGTGG |
| | GGGTCTATAGGATATACGTTTCCCGCA |
| | GCTCTTGGAAGCCAGATCGCCGATAAG |
| | GAGAGCAGACACCTGTTGTTCATCGGG |
| | GACGGCTCGTTGCAGCTGACTGTTCAG |
| | GAACTGGGGTTGGCGATCAGAGAGAAG |
| | ATTAATCCCATTTGCTTTATCATAAAT |
| | AATGATGGTTATACCGTAGAACGTGAG |
| | ATTCATGGACCTAATCAGAGCTATAAT |
| | GACATTCCTATGTGGAACTATTCAAAA |
| | TTGCCAGAGAGTTTTGGTGCAACTGAG |
| | GATCGCGTTGTTAGTAAAATAGTCCGC |
| | ACGGAGAACGAGTTTGTCAGCGTAATG |
| | AAGGAGGCCCAAGCGGACCCTAATCGG |
| | ATGTACTGGATCGAACTTATTCTGGCT |
| | AAAGAAGGAGCACCTAAAGTTTTAAAG |
| | AAGATGGGAAAACTTTTTgctgaacaa |
| | aataaatcataataagaaggagatata |
| | catatgtctattccaGAAACGCAGAAA |
| | GCCATCATATTTTATGAATCGAACGGA |
| | AAACTTGAGCACAAGGACATCCCCGTC |
| | CCGAAGCCAAAACCTAATGAGTTGCTT |
| | ATCAACGTTAAGTATTCGGGCGTATGC |
| | CACACAGACTTGCACGCATGGCACGGG |
| | GATTGGCCCTTACCGACTAAGTTGCCG |
| | TTAGTGGGCGGACATGAGGGGGCGGGA |
| | GTCGTAGTGGGAATGGGAGAGAACGTG |
| | AAGGGTTGGAAGATTGGAGATTATGCT |
| | GGGATTAAGTGGTTGAATGGGAGCTGC |
| | ATGGCCTGCGAATATTGTGAACTTGGA |
| | AATGAGAGCAATTGCCCACATGCTGAC |
| | TTGTCCGGTTACACACATGACGGTTCA |
| | TTCCAGGAATATGCTACGGCTGATGCA |
| | GTCCAAGCAGCGCATATCCCGCAAGGG |
| | ACGGACTTAGCAGAAGTAGCGCCCATT |
| | CTTTGCGCTGGGATCACCGTATATAAA |
| | GCGTTAAAGAGCGCAAATTTACGGGCC |
| | GGACATTGGGCGGCGATCAGCGGGGCC |
| | GCAGGGGGGCTGGGCAGCTTGGCCGTC |
| | CAGTACGCTAAAGCTATGGGTTATCGG |
| | GTTTTGGGCATTGACGGAGGACCGGGA |
| | AAGGAGGAATTATTCACGTCCTTGGGA |
| | GGAGAGGTATTCATTGACTTTACCAAG |
| | GAAAAAGATATCGTCTCTGCTGTAGTA |
| | AAGGCTACCAATGGCGGTGCCCACGGA |
| | ATCATAAATGTTTCAGTTTCTGAAGCG |
| | GCGATCGAAGCGTCCACTAGATATTGC |
| | CGTGCAAATGGGACAGTCGTACTTGTA |
| | GGACTTCCGGCTGGCGCCAAATGCAGC |
| | TCCGATGTATTTAATCATGTCGTGAAG |
| | TCAATCTCTATCGTTGGTTCATATGTA |
| | GGAAACCGCGCCGATACTCGTGAGGCT |
| | CTTGACTTTTTTGCCAGAGGCCTGGTT |
| | AAGTCCCCCATAAAAGTTGTTGGCTTA |
| | TCCAGCTTACCCGAAATATACGAGAAG |
| | ATGGAGAAGGGCCAGATCGCGGGGAGA |
| | tacgttgagacacttctaaataataag |
| | aaggagatatacatatgacccatcaat |
| | taagatcgcgcgatatcatcgctctgg |
| | gctttatgacatttgcgttgttcgtcg |
| | gcgcaggtaacattattttccctccaa |
| | tggtcggcttgcaggcaggcgaacacg |
| | tctggactgcggcattcggcacctcat |
| | tactgccgaggcctaccggtattaacg |
| | gtagtggcgctggcaaaagaggcggcg |
| | gtgagacagtctcagcacgccaattgg |
| | taaagtcgctggcgtactgctggcaac |
| | agtagttacctggcggtggggccgcta |
| | ttgctacgccgcgtacagctaccgatc |
| | attgaagtgggcattgcgccgctgacg |
| | ggtgattccgcgctgccgctgatattt |
| | acagcctggtctatttcgctatcgtta |
| | ttctggatcgctctatccgggcaagct |
| | gctggataccgtgggcaacttccagcg |
| | ccgctgaaaattatcgcgctggtcatc |
| | ctgtctgagccgcaattatctggccgg |
| | cgggactatcagtacggcgactgaggc |
| | ttatcaaaacgctgcgttactaacggc |
| | ttcgtcaacggctatctgaccatggat |
| | acgctgggcgcaatggtgtaggtatcg |
| | ttattgttaacgcggcgcgactcgtgg |
| | cgttaccgaagcgcgtctgctgacccg |
| | ttataccgtctgggctggcctgatggc |
| | gggtgaggtctgactctgctgtacctg |
| | gcgctgaccgtctgggttcagacagcg |
| | cgtcgctggtcgatcagtctgcaaacg |
| | gtgcggcgatcctgcatgcttacgttc |
| | agcatacctaggcggcggcggtagatc |
| | ctgctggcggcgttaatcttcatcgcc |
| | tgcctggtcacggcggttggcctgacc |
| | tgtgcttgtgcagaattcttcgcccag |
| | tacgtaccgctctcttatcgtacgctg |
| | gtgtttatcctcggcggcttctcgatg |
| | gtggtgtctaacctcggcttgagccag |
| | ctgattcagatctctgtaccggtgctg |
| | accgccatttatccgccgtgtatcgca |
| | ctggttgtattaagttttacacgctca |
| | tggtggcataattcgtcccgcgtgatt |
| | gctccgccgatgatatcagcctgctat |
| | tggtattctcgacgggatcaaggcatc |
| | tgcattcagcgatatcttaccgtcctg |
| | ggcgcagcgataccgctggccgaacaa |
| | ggtctggcgtggttaatgccaacagtg |
| | gtgatggtggttctggccattatctgg |
| | gatcgtgcggcaggtcgtcaggtgacc |
| | tccagcgctcactaatacgcatggcat |
| | ggatgaCCGATGGTAGTGTGGGGTCTC |
| | CCCATGCGAGAGTAGGGAACTGCCAGG |
| | CATCAAATAAAACGAAAGGCTCAGTCG |
| | AAAGACTGGGCCTTTCGTTTTATCTGT |
| | TGTTTGTCGGTGAACGCTCTCCTGAGT |
| | AGGACAAATCCGCCGGGAGCGGATTTG |
| | AACGTTGCGAAGCAACGGCCCGGAGGG |
| | TGGCGGGCAGGACGCCCGCCATAAACT |
| | GCCAGGCATCAAATTAAGCAGAAGGCC |
| | ATCCTGACGGATGGCCTTTTTGCGTGG |
| | CCAGTGCCAAGCTTGCATGCGTGC |
| Tet-LeuDH- | ttaagacccactacacatttaagttga |
| kivD-padA- | tactaatccgcatatgatcaattcaag |
| brnQ | gccgaataagaaggctggctctgcacc |
| construct | ttggtgatcaaataattcgatagcttg |
| (tet | tcgtaataatggcggcatactatcagt |
| Repressor is | agtaggtgtttccctttcttctttagc |
| in reverse | gacttgatgctcttgatcttccaatac |
| orientation | gcaacctaaagtaaaatgccccacagc |
| and | gctgagtgcatataatgcattctctag |
| underlined; | tgaaaaaccttgttggcataaaaaggc |
| tet promoter | taattgattttcgagagtttcatactg |
| with RBS and | tttttctgtaggccgtgtacctaaatg |
| leader region | tacttttgctccatcgcgatgacttag |
| is in bold | taaagcacatctaaaacttttagcgtt |
| italics) | attacgtaaaaaatcttgccagctttc |
| SEQ ID NO: | cccttctaaagggcaaaagtgagtatg |
| 124 | gtgcctatctaacatctcaatggctaa |
| | ggcgtcgagcaaagcccgcttattatt |
| | acatgccaatacaatgtaggctgctct |
| | acacctagatctgggcgagtttacggg |
| | ttgttaaaccttcgattccgacctcat |
| | taagcagctctaatgcgctgttaatca |
| | ctttacttttatctaatctagacat |
| | [symbol] |
| | [symbol] |
| | [symbol] |
| | [symbol] |
| | [symbol] |
| | [symbol] |
| | ATGACTCTTGAAATCTTTGAATATTTA |
| | GAAAAGTACGACTACGAGCAGGTTGTA |
| | TTTTGTCAAGACAAGGAGTCTGGGCTG |
| | AAGGCCATCATTGCCATCCACGACACA |
| | ACCTTAGGCCCGGCGCTTGGCGGAACC |
| | CGCATGTGGACCTACGACTCCGAGGAG |
| | GCGGCCATCGAGGACGCACTTCGTCTT |
| | GCTAAGGGTATGACCTATAAGAACGCG |
| | GCAGCCGGTCTGAATCTGGGGGGTGCT |
| | AAGACTGTAATCATCGGTGATCCACGC |
| | AAGGATAAGAGTGAAGCAATGTTTCGC |
| | GCTTTAGGGCGCTATATTCAGGGCTTG |
| | AACGGCCGCTACATTACCGCAGAAGAC |
| | GTAGGGACAACAGTAGACGACATGGAC |
| | ATCATCCATGAGGAAACTGATTTCGTG |
| | ACCGGTATTTCACCTTCATTCGGGTCA |
| | TCCGGTAACCCTTCCCCCGTAACAGCC |
| | TATGGGGTTTATCGCGGAATGAAGGCC |
| | GCAGCCAAGGAGGCATTTGGCACTGAC |
| | AATTTAGAAGGAAAAGTAATTGCTGTC |
| | CAAGGCGTGGGCAATGTGGCCTACCAT |
| | TTGTGTAAACACCTTCACGCGGAAGGT |
| | GCAAAATTGATCGTTACGGATATTAAC |
| | AAGGAGGCAGTCCAGCGCGCTGTAGAG |
| | GAATTTGGAGCATCGGCTGTGGAACCA |
| | AATGAGATCTACGGTGTAGAATGTGAC |
| | ATTTACGCTCCATGCGCACTTGGTGCC |
| | ACGGTGAATGACGAGACCATCCCCCAA |
| | CTTAAGGCGAAGGTAATCGCTGGTTCA |
| | GCTAACAACCAATTAAAAGAGGACCGT |
| | CACGGAGATATCATCCACGAAATGGGT |
| | ATCGTGTACGCCCCCGATTATGTTATC |
| | AACGCGGGCGGCGTAATCAACGTAGCC |
| | GATGAGCTTTATGGATACAACCGCGAA |
| | CGTGCGCTGAAACGCGTGGAAAGCATT |
| | TATGACACGATCGCAAAGGTAATCGAG |
| | ATCAGTAAGCGCGACGGCATTGCGACA |
| | TACGTGGCAGCGGACCGTCTGGCCGAA |
| | GAACGCATCGCGAGTTTGAAGAATAGC |
| | CGTAGTACCTACTTGCGCAACGGGCAC |
| | GATATTATCAGCCGTCGCtgataagaa |
| | ggagatatacatatgtatacagtagga |
| | GATTACTTATTGGACCGGTTGCACGAA |
| | CTTGGAATTGAGGAAATTTTTGGAGTT |
| | CCGGGTGACTACAACCTGCAGTTCCTT |
| | GACCAAATCATCTCCCATAAGGACATG |
| | AAATGGGTCGGCAATGCCAATGAGCTG |
| | AACGCATCATATATGGCAGACGGGTAT |
| | GCTCGGACCAAAAAGGCTGCAGCATTC |
| | CTTACCACGTTTGGCGTGGGGGAATTA |
| | AGTGCTGTAAATGGACTGGCAGGATCC |
| | TATGCGGAGAATTTACCGGTAGTCGAA |
| | ATTGTTGGCTCGCCTACGTCCAAGGTG |
| | CAGAATGAGGGGAAATTCGTCCATCAC |
| | ACACTTGCAGACGGTGATTTTAAGCAC |
| | TTTATGAAGATGCATGAGCCGGTAACG |
| | GCTGCGCGGACGCTTCTTACTGCGGAA |
| | AACGCAACAGTAGAGATTGATCGCGTT |
| | CTGAGCGCACTGCTTAAGGAACGGAAG |
| | CCCGTCTATATTAACTTACCGGTAGAC |
| | GTGGCCGCAGCCAAAGCCGAAAAACCA |
| | AGCCTGCCTCTTAAGAAGGAGAATTCC |
| | ACGTCCAACACCAGTGACCAAGAGATT |
| | TTGAACAAAATTCAAGAGTCTTTGAAG |
| | AACGCGAAGAAGCCCATCGTAATTACA |
| | GGACATGAGATTATCTCGTTTGGCCTG |
| | GAGAAAACGGTTACACAGTTTATTTCC |
| | AAAACGAAGTTACCTATAACGACGTTA |
| | AACTTTGGAAAGAGCTCTGTGGATGAG |
| | GCACTTCCTAGTTTCTTAGGAATCTAT |
| | AATGGGACCCTTTCAGAGCCAAACTTA |
| | AAGGAATTCGTTGAAAGTGCGGATTTT |
| | ATCTTAATGCTTGGGGTTAAATTGACT |
| | GATTCCAGCACCGGAGCTTTTACGCAC |
| | CATTTAAACGAGAACAAAATGATCTCT |
| | TTGAATATCGACGAAGGCAAAATTTTT |
| | AATGAAAGAATTCAGAACTTTGATTTT |
| | GAATCCCTTATTAGTTCACTTTTAGAT |
| | TTAAGTGAAATAGAGTATAAGGGAAAG |
| | TATATAGACAAGAAGCAAGAGGATTTC |
| | GTTCCGTCTAATGCTCTTTTAAGTCAA |
| | GACAGACTTTGGCAGGCGGTTGAGAAC |
| | CTTACACAATCCAATGAAACGATAGTC |
| | GCCGAACAAGGGACCAGTTTCTTCGGC |
| | GCTTCTTCCATATTCCTGAAGTCTAAG |
| | TCTCATTTCATTGGACAGCCCCTGTGG |
| | GGGTCTATAGGATATACGTTTCCCGCA |
| | GCTCTTGGAAGCCAGATCGCCGATAAG |
| | GAGAGCAGACACCTGTTGTTCATCGGG |
| | GACGGCTCGTTGCAGCTGACTGTTCAG |
| | GAACTGGGGTTGGCGATCAGAGAGAAG |
| | ATTAATCCCATTTGCTTTATCATAAAT |
| | AATGATGGTTATACCGTAGAACGTGAG |
| | ATTCATGGACCTAATCAGAGCTATAAT |
| | GACATTCCTATGTGGAACTATTCAAAA |
| | TTGCCAGAGAGTTTTGGTGCAACTGAG |
| | GATCGCGTTGTTAGTAAAATAGTCCGC |
| | ACGGAGAACGAGTTTGTCAGCGTAATG |
| | AAGGAGGCCCAAGCGGACCCTAATCGG |
| | ATGTACTGGATCGAACTTATTCTGGCT |
| | AAAGAAGGAGCACCTAAAGTTTTAAAG |
| | AAGATGGGAAAACTTTTTgctgaacaa |
| | aataaatcataataagaaggagatata |
| | catATGACAGAGCCGCATGTAGCAGTA |
| | TTAAGCCAGGTCCAACAGTTTCTCGAT |
| | CGTCAACACGGTCTTTATATTGATGGT |
| | CGTCCTGGCCCCGCACAAAGTGAAAAA |
| | CGGTTGGCGATCTTTGATCCGGCCACC |
| | GGGCAAGAAATTGCGTCTACTGCTGAT |
| | GCCAACGAAGCGGATGTAGATAACGCA |
| | GTCATGTCTGCCTGGCGGGCCTTTGTC |
| | TCGCGTCGCTGGGCCGGGCGATTACCC |
| | GCAGAGCGTGAACGTATTCTGCTACGT |
| | TTTGCTGATCTGGTGGAGCAGCACAGT |
| | GAGGAGCTGGCGCAACTGGAAACCCTG |
| | GAGCAAGGCAAGTCAATTGCCATTTCC |
| | CGTGCTTTTGAAGTGGGCTGTACGCTG |
| | AACTGGATGCGTTATACCGCCGGGTTA |
| | ACGACCAAAATCGCGGGTAAAACGCTG |
| | GACTTGTCGATTCCCTTACCCCAGGGG |
| | GCGCGTTATCAGGCCTGGACGCGTAAA |
| | GAGCCGGTTGGCGTAGTGGCGGGAATT |
| | GTGCCATGGAACTTTCCGTTGATGATT |
| | GGTATGTGGAAGGTGATGCCAGCACTG |
| | GCAGCAGGCTGTTCAATCGTGATTAAG |
| | CCTTCGGAAACCACGCCACTGACGATG |
| | TTGCGCGTGGCGGAACTGGCCAGCGAG |
| | GCTGGTATCCCTGATGGCGTTTTTAAT |
| | GTCGTCACCGGGTCAGGTGCTGTATGC |
| | GGCGCGGCCCTGACGTCACATCCTCAT |
| | GTTGCGAAAATCAGTTTTACCGGTTCA |
| | ACCGCGACGGGAAAAGGTATTGCCAGA |
| | ACTGCTGCTGATCACTTAACGCGTGTA |
| | ACGCTGGAACTGGGCGGTAAAAACCCG |
| | GCAATTGTATTAAAAGATGCTGATCCG |
| | CAATGGGTTATTGAAGGCTTGATGACC |
| | GGAAGCTTCCTGAATCAAGGGCAAGTA |
| | TGCGCCGCCAGTTCGCGAATTTATATT |
| | GAAGCGCCGTTGTTTGACACGCTGGTT |
| | AGTGGATTTGAGCAGGCGGTAAAATCG |
| | TTGCAAGTGGGACCGGGGATGTCACCT |
| | GTTGCACAGATTAACCCTTTGGTTTCT |
| | CGTGCGCACTGCGACAAAGTGTGTTCA |
| | TTCCTCGACGATGCGCAGGCACAGCAA |
| | GCAGAGCTGATTCGCGGGTCGAATGGA |
| | CCAGCCGGAGAGGGGTATTATGTTGCG |
| | CCAACGCTGGTGGTAAATCCCGATGCT |
| | AAATTGCGCTTAACTCGTGAAGAGGTG |
| | TTTGGTCCGGTGGTAAACCTGGTGCGA |
| | GTAGCGGATGGAGAAGAGGCGTTACAA |
| | CTGGCAAACGACACGGAATATGGCTTA |
| | ACTGCCAGTGTCTGGACGCAAAATCTC |
| | TCCCAGGCTCTGGAATATAGCGATCGC |
| | TTACAGGCAGGGACGGTGTGGGTAAAC |
| | AGCCATACCTTAATTGACGCTAACTTA |
| | CCGTTTGGTGGGATGAAGCAGTCAGGA |
| | ACGGGCCGTGATTTTGGCCCCGACTGG |
| | CTGGACGGTTGGTGTGAAACTAAGTCG |
| | GTGTGTGTACGGTATTAAtaagaagga |
| | gatatacatatgacccatcaattaaga |
| | tcgcgcgatatcatcgctctgggcata |
| | tgacatttgcgttgacgtcggcgcagg |
| | taacattattaccctccaatggtcggc |
| | ttgcaggcaggcgaacacgtctggact |
| | gcggcattcggcacctcattactgccg |
| | aggcctaccggtattaacggtagtggc |
| | gctggcaaaagaggcggcggtgagaca |
| | gtctcagcacgccaattggtaaagtcg |
| | ctggcgtactgctggcaacagtagtta |
| | cctggcggtggggccgctattgctacg |
| | ccgcgtacagctaccgatcattgaagt |
| | gggcattgcgccgctgacgggtgattc |
| | cgcgctgccgctgatatttacagcctg |
| | gtctatttcgctatcgttattctggat |
| | cgctctatccgggcaagctgctggata |
| | ccgtgggcaacttccttgcgccgctga |
| | aaattatcgcgctggtcatcctgtctg |
| | agccgcaattatctggccggcgggact |
| | atcagtacggcgactgaggcttatcaa |
| | aacgctgcgttactaacggcttcgtca |
| | acggctatctgaccatggatacgctgg |
| | gcgcaatggtgtaggtatcgttattgt |
| | taacgcggcgcgactcgtggcgttacc |
| | gaagcgcgtctgctgacccgttatacc |
| | gtctgggctggcctgatggcgggtgag |
| | gtctgactctgctgtacctggcgctga |
| | ccgtctgggttcagacagcgcgtcgct |
| | ggtcgatcagtctgcaaacggtgcggc |
| | gatcctgcatgcttacgttcagcatac |
| | catggcggcggcggtagcacctgctgg |
| | cggcgttaatcttcatcgcctgcctgg |
| | tcacggcggaggcctgacctgtgcttg |
| | tgcagaattcacgcccagtacgtaccg |
| | ctctcttatcgtacgctggtgatatcc |
| | tcggcggcttctcgatggtggtgtcta |
| | acctcggcttgagccagctgattcaga |
| | tctctgtaccggtgctgaccgccattt |
| | atccgccgtgtatcgcactggagtatt |
| | aagattacacgctcatggtggcataat |
| | tcgtcccgcgtgattgctccgccgatg |
| | atatcagcctgctattggtattctcga |
| | cgggatcaaggcatctgcattcagcga |
| | tatcttaccgtcctgggcgcagcgata |
| | ccgctggccgaacaaggtctggcgtgg |
| | ttaatgccaacagtggtgatggtggac |
| | tggccattatctgggatcgtgcggcag |
| | gtcgtcaggtgacctccagcgctcact |
| | aatacgcatggcatggatgaCCGATGG |
| | TAGTGTGGGGTCTCCCCATGCGAGAGT |
| | AGGGAACTGCCAGGCATCAAATAAAAC |
| | GAAAGGCTCAGTCGAAAGACTGGGCCT |
| | TTCGTTTTATCTGTTGTTTGTCGGTGA |
| | ACGCTCTCCTGAGTAGGACAAATCCGC |
| | CGGGAGCGGATTTGAACGTTGCGAAGC |
| | AACGGCCCGGAGGGTGGCGGGCAGGAC |
| | GCCCGCCATAAACTGCCAGGCATCAAA |
| | TTAAGCAGAAGGCCATCCTGACGGATG |
| | GCCTTTTTGCGTGGCCAGTGCCAAGCT |
| | TGCATGCGTGC |
| LeuDH-kivD- | ATGACTCTTGAAATCTTTGAATATTTA |
| padA-brnQ | GAAAAGTACGACTACGAGCAGGTTGTA |
| (RBS are | TTTTGTCAAGACAAGGAGTCTGGGCTG |
| underlined) | AAGGCCATCATTGCCATCCACGACACA |
| SEQ ID NO: | ACCTTAGGCCCGGCGCTTGGCGGAACC |
| 125 | CGCATGTGGACCTACGACTCCGAGGAG |
| | GCGGCCATCGAGGACGCACTTCGTCTT |
| | GCTAAGGGTATGACCTATAAGAACGCG |
| | GCAGCCGGTCTGAATCTGGGGGGTGCT |
| | AAGACTGTAATCATCGGTGATCCACGC |
| | AAGGATAAGAGTGAAGCAATGTTTCGC |
| | GCTTTAGGGCGCTATATTCAGGGCTTG |
| | AACGGCCGCTACATTACCGCAGAAGAC |
| | GTAGGGACAACAGTAGACGACATGGAC |
| | ATCATCCATGAGGAAACTGATTTCGTG |
| | ACCGGTATTTCACCTTCATTCGGGTCA |
| | TCCGGTAACCCTTCCCCCGTAACAGCC |
| | TATGGGGTTTATCGCGGAATGAAGGCC |
| | GCAGCCAAGGAGGCATTTGGCACTGAC |
| | AATTTAGAAGGAAAAGTAATTGCTGTC |
| | CAAGGCGTGGGCAATGTGGCCTACCAT |
| | TTGTGTAAACACCTTCACGCGGAAGGT |
| | GCAAAATTGATCGTTACGGATATTAAC |
| | AAGGAGGCAGTCCAGCGCGCTGTAGAG |
| | GAATTTGGAGCATCGGCTGTGGAACCA |
| | AATGAGATCTACGGTGTAGAATGTGAC |
| | ATTTACGCTCCATGCGCACTTGGTGCC |
| | ACGGTGAATGACGAGACCATCCCCCAA |
| | CTTAAGGCGAAGGTAATCGCTGGTTCA |
| | GCTAACAACCAATTAAAAGAGGACCGT |
| | CACGGAGATATCATCCACGAAATGGGT |
| | ATCGTGTACGCCCCCGATTATGTTATC |
| | AACGCGGGCGGCGTAATCAACGTAGCC |
| | GATGAGCTTTATGGATACAACCGCGAA |
| | CGTGCGCTGAAACGCGTGGAAAGCATT |
| | TATGACACGATCGCAAAGGTAATCGAG |
| | ATCAGTAAGCGCGACGGCATTGCGACA |
| | TACGTGGCAGCGGACCGTCTGGCCGAA |
| | GAACGCATCGCGAGTTTGAAGAATAGC |
| | CGTAGTACCTACTTGCGCAACGGGCAC |
| | GATATTATCAGCCGTCGCtgataagaa |
| | ggagatatacatatgtatacagtagga |
| | GATTACTTATTGGACCGGTTGCACGAA |
| | CTTGGAATTGAGGAAATTTTTGGAGTT |
| | CCGGGTGACTACAACCTGCAGTTCCTT |
| | GACCAAATCATCTCCCATAAGGACATG |
| | AAATGGGTCGGCAATGCCAATGAGCTG |
| | AACGCATCATATATGGCAGACGGGTAT |
| | GCTCGGACCAAAAAGGCTGCAGCATTC |
| | CTTACCACGTTTGGCGTGGGGGAATTA |
| | AGTGCTGTAAATGGACTGGCAGGATCC |
| | TATGCGGAGAATTTACCGGTAGTCGAA |
| | ATTGTTGGCTCGCCTACGTCCAAGGTG |
| | CAGAATGAGGGGAAATTCGTCCATCAC |
| | ACACTTGCAGACGGTGATTTTAAGCAC |
| | TTTATGAAGATGCATGAGCCGGTAACG |
| | GCTGCGCGGACGCTTCTTACTGCGGAA |
| | AACGCAACAGTAGAGATTGATCGCGTT |
| | CTGAGCGCACTGCTTAAGGAACGGAAG |
| | CCCGTCTATATTAACTTACCGGTAGAC |
| | GTGGCCGCAGCCAAAGCCGAAAAACCA |
| | AGCCTGCCTCTTAAGAAGGAGAATTCC |
| | ACGTCCAACACCAGTGACCAAGAGATT |
| | TTGAACAAAATTCAAGAGTCTTTGAAG |
| | AACGCGAAGAAGCCCATCGTAATTACA |
| | GGACATGAGATTATCTCGTTTGGCCTG |
| | GAGAAAACGGTTACACAGTTTATTTCC |
| | AAAACGAAGTTACCTATAACGACGTTA |
| | AACTTTGGAAAGAGCTCTGTGGATGAG |
| | GCACTTCCTAGTTTCTTAGGAATCTAT |
| | AATGGGACCCTTTCAGAGCCAAACTTA |
| | AAGGAATTCGTTGAAAGTGCGGATTTT |
| | ATCTTAATGCTTGGGGTTAAATTGACT |
| | GATTCCAGCACCGGAGCTTTTACGCAC |
| | CATTTAAACGAGAACAAAATGATCTCT |
| | TTGAATATCGACGAAGGCAAAATTTTT |
| | AATGAAAGAATTCAGAACTTTGATTTT |
| | GAATCCCTTATTAGTTCACTTTTAGAT |
| | TTAAGTGAAATAGAGTATAAGGGAAAG |
| | TATATAGACAAGAAGCAAGAGGATTTC |
| | GTTCCGTCTAATGCTCTTTTAAGTCAA |
| | GACAGACTTTGGCAGGCGGTTGAGAAC |
| | CTTACACAATCCAATGAAACGATAGTC |
| | GCCGAACAAGGGACCAGTTTCTTCGGC |
| | GCTTCTTCCATATTCCTGAAGTCTAAG |
| | TCTCATTTCATTGGACAGCCCCTGTGG |
| | GGGTCTATAGGATATACGTTTCCCGCA |
| | GCTCTTGGAAGCCAGATCGCCGATAAG |
| | GAGAGCAGACACCTGTTGTTCATCGGG |
| | GACGGCTCGTTGCAGCTGACTGTTCAG |
| | GAACTGGGGTTGGCGATCAGAGAGAAG |
| | ATTAATCCCATTTGCTTTATCATAAAT |
| | AATGATGGTTATACCGTAGAACGTGAG |
| | ATTCATGGACCTAATCAGAGCTATAAT |
| | GACATTCCTATGTGGAACTATTCAAAA |
| | TTGCCAGAGAGTTTTGGTGCAACTGAG |
| | GATCGCGTTGTTAGTAAAATAGTCCGC |
| | ACGGAGAACGAGTTTGTCAGCGTAATG |
| | AAGGAGGCCCAAGCGGACCCTAATCGG |
| | ATGTACTGGATCGAACTTATTCTGGCT |
| | AAAGAAGGAGCACCTAAAGTTTTAAAG |
| | AAGATGGGAAAACTTTTTgctgaacaa |
| | aataaatcataataagaaggagatata |
| | catATGACAGAGCCGCATGTAGCAGTA |
| | TTAAGCCAGGTCCAACAGTTTCTCGAT |
| | CGTCAACACGGTCTTTATATTGATGGT |
| | CGTCCTGGCCCCGCACAAAGTGAAAAA |
| | CGGTTGGCGATCTTTGATCCGGCCACC |
| | GGGCAAGAAATTGCGTCTACTGCTGAT |
| | GCCAACGAAGCGGATGTAGATAACGCA |
| | GTCATGTCTGCCTGGCGGGCCTTTGTC |
| | TCGCGTCGCTGGGCCGGGCGATTACCC |
| | GCAGAGCGTGAACGTATTCTGCTACGT |
| | TTTGCTGATCTGGTGGAGCAGCACAGT |
| | GAGGAGCTGGCGCAACTGGAAACCCTG |
| | GAGCAAGGCAAGTCAATTGCCATTTCC |
| | CGTGCTTTTGAAGTGGGCTGTACGCTG |
| | AACTGGATGCGTTATACCGCCGGGTTA |
| | ACGACCAAAATCGCGGGTAAAACGCTG |
| | GACTTGTCGATTCCCTTACCCCAGGGG |
| | GCGCGTTATCAGGCCTGGACGCGTAAA |
| | GAGCCGGTTGGCGTAGTGGCGGGAATT |
| | GTGCCATGGAACTTTCCGTTGATGATT |
| | GGTATGTGGAAGGTGATGCCAGCACTG |
| | GCAGCAGGCTGTTCAATCGTGATTAAG |
| | CCTTCGGAAACCACGCCACTGACGATG |
| | TTGCGCGTGGCGGAACTGGCCAGCGAG |
| | GCTGGTATCCCTGATGGCGTTTTTAAT |
| | GTCGTCACCGGGTCAGGTGCTGTATGC |
| | GGCGCGGCCCTGACGTCACATCCTCAT |
| | GTTGCGAAAATCAGTTTTACCGGTTCA |
| | ACCGCGACGGGAAAAGGTATTGCCAGA |
| | ACTGCTGCTGATCACTTAACGCGTGTA |
| | ACGCTGGAACTGGGCGGTAAAAACCCG |
| | GCAATTGTATTAAAAGATGCTGATCCG |
| | CAATGGGTTATTGAAGGCTTGATGACC |
| | GGAAGCTTCCTGAATCAAGGGCAAGTA |
| | TGCGCCGCCAGTTCGCGAATTTATATT |
| | GAAGCGCCGTTGTTTGACACGCTGGTT |
| | AGTGGATTTGAGCAGGCGGTAAAATCG |
| | TTGCAAGTGGGACCGGGGATGTCACCT |
| | GTTGCACAGATTAACCCTTTGGTTTCT |
| | CGTGCGCACTGCGACAAAGTGTGTTCA |
| | TTCCTCGACGATGCGCAGGCACAGCAA |
| | GCAGAGCTGATTCGCGGGTCGAATGGA |
| | CCAGCCGGAGAGGGGTATTATGTTGCG |
| | CCAACGCTGGTGGTAAATCCCGATGCT |
| | AAATTGCGCTTAACTCGTGAAGAGGTG |
| | TTTGGTCCGGTGGTAAACCTGGTGCGA |
| | GTAGCGGATGGAGAAGAGGCGTTACAA |
| | CTGGCAAACGACACGGAATATGGCTTA |
| | ACTGCCAGTGTCTGGACGCAAAATCTC |
| | TCCCAGGCTCTGGAATATAGCGATCGC |
| | TTACAGGCAGGGACGGTGTGGGTAAAC |
| | AGCCATACCTTAATTGACGCTAACTTA |
| | CCGTTTGGTGGGATGAAGCAGTCAGGA |
| | ACGGGCCGTGATTTTGGCCCCGACTGG |
| | CTGGACGGTTGGTGTGAAACTAAGTCG |
| | GTGTGTGTACGGTATTAAtaagaagga |
| | gatatacatatgacccatcaattaaga |
| | tcgcgcgatatcatcgctctgggcttt |
| | atgacatttgcgttgacgtcggcgcag |
| | gtaacattattaccctccaatggtcgg |
| | cttgcaggcaggcgaacacgtctggac |
| | tgcggcattcggcacctcattactgcc |
| | gaggcctaccggtattaacggtagtgg |
| | cgctggcaaaagaggcggcggtgagac |
| | agtctcagcacgccaattggtaaagtc |
| | gctggcgtactgctggcaacagtagtt |
| | acctggcggtggggccgctattgctac |
| | gccgcgtacagctaccgatcattgaag |
| | tgggcattgcgccgctgacgggtgatt |
| | ccgcgctgccgctgatatttacagcct |
| | ggtctatttcgctatcgttattctgga |
| | tcgctctatccgggcaagctgctggat |
| | accgtgggcaacttccagcgccgctga |
| | aaattatcgcgctggtcatcctgtctg |
| | agccgcaattatctggccggcgggttc |
| | tatcagtacggcgactgaggcttatca |
| | aaacgctgcgttttctaacggcttcgt |
| | caacggctatctgaccatggatacgct |
| | gggcgcaatggtgtaggtatcgttatt |
| | gttaacgcggcgcgactcgtggcgtta |
| | ccgaagcgcgtctgctgacccgttata |
| | ccgtctgggctggcctgatggcgggtg |
| | aggtctgactctgctgtacctggcgct |
| | gaccgtctgggttcagacagcgcgtcg |
| | ctggtcgatcagtctgcaaacggtgcg |
| | gcgatcctgcatgcttacgttcagcat |
| | acctaggcggcggcggtagatcctgct |
| | ggcggcgttaatcttcatcgcctgcct |
| | ggtcacggcggaggcctgacctgtgat |
| | gtgcagaattatcgcccagtacgtacc |
| | gctctcttatcgtacgctggtgatatc |
| | ctcggcggcactcgatggtggtgtcta |
| | acctcggcttgagccagctgattcaga |
| | tctctgtaccggtgctgaccgccattt |
| | atccgccgtgtatcgcactggagtatt |
| | aagattacacgctcatggtggcataat |
| | tcgtcccgcgtgattgctccgccgatg |
| | atatcagcctgctattggtattctcga |
| | cgggatcaaggcatctgcattcagcga |
| | tatcttaccgtcctgggcgcagcgata |
| | ccgctggccgaacaaggtctggcgtgg |
| | ttaatgccaacagtggtgatggtggac |
| | tggccattatctgggatcgtgcggcag |
| | gtcgtcaggtgacctccagcgctcact |
| | aa |
| Fnrs-LeuDH- | AGTTGTTCTTATTGGTGGTGTTGCTTT |
| kivD-padA- | ATGGTTGCATCGTAGTAAATGGTTGTA |
| brnQ (RBS | ACAAAAGCAATTTTTCCGGCTGTCTGT |
| are | ATACAAAAACGCCGCAAAGTTTGAGCG |
| underlined); | AAGTCAATAAACTCTCTACCCATTCAG |
| FNR | GGCAATATCTCTCTTggatccaaagtg |
| promoter with | aactctagaaataattagataacttta |
| RBS and | agaaggagatatacatATGACTCTTGA |
| leader region | AATCTTTGAATATTTAGAAAAGTACGA |
| (underlined), | CTACGAGCAGGTTGTATTTTGTCAAGA |
| FNR binding | CAAGGAGTCTGGGCTGAAGGCCATCAT |
| site bold | TGCCATCCACGACACAACCTTAGGCCC |
| SEQ ID NO: | GGCGCTTGGCGGAACCCGCATGTGGAC |
| 126 | CTACGACTCCGAGGAGGCGGCCATCGA |
| | GGACGCACTTCGTCTTGCTAAGGGTAT |
| | GACCTATAAGAACGCGGCAGCCGGTCT |
| | GAATCTGGGGGGTGCTAAGACTGTAAT |
| | CATCGGTGATCCACGCAAGGATAAGAG |
| | TGAAGCAATGTTTCGCGCTTTAGGGCG |
| | CTATATTCAGGGCTTGAACGGCCGCTA |
| | CATTACCGCAGAAGACGTAGGGACAAC |
| | AGTAGACGACATGGACATCATCCATGA |
| | GGAAACTGATTTCGTGACCGGTATTTC |
| | ACCTTCATTCGGGTCATCCGGTAACCC |
| | TTCCCCCGTAACAGCCTATGGGGTTTA |
| | TCGCGGAATGAAGGCCGCAGCCAAGGA |
| | GGCATTTGGCACTGACAATTTAGAAGG |
| | AAAAGTAATTGCTGTCCAAGGCGTGGG |
| | CAATGTGGCCTACCATTTGTGTAAACA |
| | CCTTCACGCGGAAGGTGCAAAATTGAT |
| | CGTTACGGATATTAACAAGGAGGCAGT |
| | CCAGCGCGCTGTAGAGGAATTTGGAGC |
| | ATCGGCTGTGGAACCAAATGAGATCTA |
| | CGGTGTAGAATGTGACATTTACGCTCC |
| | ATGCGCACTTGGTGCCACGGTGAATGA |
| | CGAGACCATCCCCCAACTTAAGGCGAA |
| | GGTAATCGCTGGTTCAGCTAACAACCA |
| | ATTAAAAGAGGACCGTCACGGAGATAT |
| | CATCCACGAAATGGGTATCGTGTACGC |
| | CCCCGATTATGTTATCAACGCGGGCGG |
| | CGTAATCAACGTAGCCGATGAGCTTTA |
| | TGGATACAACCGCGAACGTGCGCTGAA |
| | ACGCGTGGAAAGCATTTATGACACGAT |
| | CGCAAAGGTAATCGAGATCAGTAAGCG |
| | CGACGGCATTGCGACATACGTGGCAGC |
| | GGACCGTCTGGCCGAAGAACGCATCGC |
| | GAGTTTGAAGAATAGCCGTAGTACCTA |
| | CTTGCGCAACGGGCACGATATTATCAG |
| | CCGTCGCtgataagaaggagatataca |
| | tatgtatacagtaggaGATTACTTATT |
| | GGACCGGTTGCACGAACTTGGAATTGA |
| | GGAAATTTTTGGAGTTCCGGGTGACTA |
| | CAACCTGCAGTTCCTTGACCAAATCAT |
| | CTCCCATAAGGACATGAAATGGGTCGG |
| | CAATGCCAATGAGCTGAACGCATCATA |
| | TATGGCAGACGGGTATGCTCGGACCAA |
| | AAAGGCTGCAGCATTCCTTACCACGTT |
| | TGGCGTGGGGGAATTAAGTGCTGTAAA |
| | TGGACTGGCAGGATCCTATGCGGAGAA |
| | TTTACCGGTAGTCGAAATTGTTGGCTC |
| | GCCTACGTCCAAGGTGCAGAATGAGGG |
| | GAAATTCGTCCATCACACACTTGCAGA |
| | CGGTGATTTTAAGCACTTTATGAAGAT |
| | GCATGAGCCGGTAACGGCTGCGCGGAC |
| | GCTTCTTACTGCGGAAAACGCAACAGT |
| | AGAGATTGATCGCGTTCTGAGCGCACT |
| | GCTTAAGGAACGGAAGCCCGTCTATAT |
| | TAACTTACCGGTAGACGTGGCCGCAGC |
| | CAAAGCCGAAAAACCAAGCCTGCCTCT |
| | TAAGAAGGAGAATTCCACGTCCAACAC |
| | CAGTGACCAAGAGATTTTGAACAAAAT |
| | TCAAGAGTCTTTGAAGAACGCGAAGAA |
| | GCCCATCGTAATTACAGGACATGAGAT |
| | TATCTCGTTTGGCCTGGAGAAAACGGT |
| | TACACAGTTTATTTCCAAAACGAAGTT |
| | ACCTATAACGACGTTAAACTTTGGAAA |
| | GAGCTCTGTGGATGAGGCACTTCCTAG |
| | TTTCTTAGGAATCTATAATGGGACCCT |
| | TTCAGAGCCAAACTTAAAGGAATTCGT |
| | TGAAAGTGCGGATTTTATCTTAATGCT |
| | TGGGGTTAAATTGACTGATTCCAGCAC |
| | CGGAGCTTTTACGCACCATTTAAACGA |
| | GAACAAAATGATCTCTTTGAATATCGA |
| | CGAAGGCAAAATTTTTAATGAAAGAAT |
| | TCAGAACTTTGATTTTGAATCCCTTAT |
| | TAGTTCACTTTTAGATTTAAGTGAAAT |
| | AGAGTATAAGGGAAAGTATATAGACAA |
| | GAAGCAAGAGGATTTCGTTCCGTCTAA |
| | TGCTCTTTTAAGTCAAGACAGACTTTG |
| | GCAGGCGGTTGAGAACCTTACACAATC |
| | CAATGAAACGATAGTCGCCGAACAAGG |
| | GACCAGTTTCTTCGGCGCTTCTTCCAT |
| | ATTCCTGAAGTCTAAGTCTCATTTCAT |
| | TGGACAGCCCCTGTGGGGGTCTATAGG |
| | ATATACGTTTCCCGCAGCTCTTGGAAG |
| | CCAGATCGCCGATAAGGAGAGCAGACA |
| | CCTGTTGTTCATCGGGGACGGCTCGTT |
| | GCAGCTGACTGTTCAGGAACTGGGGTT |
| | GGCGATCAGAGAGAAGATTAATCCCAT |
| | TTGCTTTATCATAAATAATGATGGTTA |
| | TACCGTAGAACGTGAGATTCATGGACC |
| | TAATCAGAGCTATAATGACATTCCTAT |
| | GTGGAACTATTCAAAATTGCCAGAGAG |
| | TTTTGGTGCAACTGAGGATCGCGTTGT |
| | TAGTAAAATAGTCCGCACGGAGAACGA |
| | GTTTGTCAGCGTAATGAAGGAGGCCCA |
| | AGCGGACCCTAATCGGATGTACTGGAT |
| | CGAACTTATTCTGGCTAAAGAAGGAGC |
| | ACCTAAAGTTTTAAAGAAGATGGGAAA |
| | ACTTTTTgctgaacaaaataaatcata |
| | ataagaaggagatatacatATGACAGA |
| | GCCGCATGTAGCAGTATTAAGCCAGGT |
| | CCAACAGTTTCTCGATCGTCAACACGG |
| | TCTTTATATTGATGGTCGTCCTGGCCC |
| | CGCACAAAGTGAAAAACGGTTGGCGAT |
| | CTTTGATCCGGCCACCGGGCAAGAAAT |
| | TGCGTCTACTGCTGATGCCAACGAAGC |
| | GGATGTAGATAACGCAGTCATGTCTGC |
| | CTGGCGGGCCTTTGTCTCGCGTCGCTG |
| | GGCCGGGCGATTACCCGCAGAGCGTGA |
| | ACGTATTCTGCTACGTTTTGCTGATCT |
| | GGTGGAGCAGCACAGTGAGGAGCTGGC |
| | GCAACTGGAAACCCTGGAGCAAGGCAA |
| | GTCAATTGCCATTTCCCGTGCTTTTGA |
| | AGTGGGCTGTACGCTGAACTGGATGCG |
| | TTATACCGCCGGGTTAACGACCAAAAT |
| | CGCGGGTAAAACGCTGGACTTGTCGAT |
| | TCCCTTACCCCAGGGGGCGCGTTATCA |
| | GGCCTGGACGCGTAAAGAGCCGGTTGG |
| | CGTAGTGGCGGGAATTGTGCCATGGAA |
| | CTTTCCGTTGATGATTGGTATGTGGAA |
| | GGTGATGCCAGCACTGGCAGCAGGCTG |
| | TTCAATCGTGATTAAGCCTTCGGAAAC |
| | CACGCCACTGACGATGTTGCGCGTGGC |
| | GGAACTGGCCAGCGAGGCTGGTATCCC |
| | TGATGGCGTTTTTAATGTCGTCACCGG |
| | GTCAGGTGCTGTATGCGGCGCGGCCCT |
| | GACGTCACATCCTCATGTTGCGAAAAT |
| | CAGTTTTACCGGTTCAACCGCGACGGG |
| | AAAAGGTATTGCCAGAACTGCTGCTGA |
| | TCACTTAACGCGTGTAACGCTGGAACT |
| | GGGCGGTAAAAACCCGGCAATTGTATT |
| | AAAAGATGCTGATCCGCAATGGGTTAT |
| | TGAAGGCTTGATGACCGGAAGCTTCCT |
| | GAATCAAGGGCAAGTATGCGCCGCCAG |
| | TTCGCGAATTTATATTGAAGCGCCGTT |
| | GTTTGACACGCTGGTTAGTGGATTTGA |
| | GCAGGCGGTAAAATCGTTGCAAGTGGG |
| | ACCGGGGATGTCACCTGTTGCACAGAT |
| | TAACCCTTTGGTTTCTCGTGCGCACTG |
| | CGACAAAGTGTGTTCATTCCTCGACGA |
| | TGCGCAGGCACAGCAAGCAGAGCTGAT |
| | TCGCGGGTCGAATGGACCAGCCGGAGA |
| | GGGGTATTATGTTGCGCCAACGCTGGT |
| | GGTAAATCCCGATGCTAAATTGCGCTT |
| | AACTCGTGAAGAGGTGTTTGGTCCGGT |
| | GGTAAACCTGGTGCGAGTAGCGGATGG |
| | AGAAGAGGCGTTACAACTGGCAAACGA |
| | CACGGAATATGGCTTAACTGCCAGTGT |
| | CTGGACGCAAAATCTCTCCCAGGCTCT |
| | GGAATATAGCGATCGCTTACAGGCAGG |
| | GACGGTGTGGGTAAACAGCCATACCTT |
| | AATTGACGCTAACTTACCGTTTGGTGG |
| | GATGAAGCAGTCAGGAACGGGCCGTGA |
| | TTTTGGCCCCGACTGGCTGGACGGTTG |
| | GTGTGAAACTAAGTCGGTGTGTGTACG |
| | GTATTAAtaagaaggagatatacatat |
| | gacccatcaattaagatcgcgcgatat |
| | catcgctctgggctttatgacatttgc |
| | gttgacgtcggcgcaggtaacattatt |
| | accctccaatggtcggcttgcaggcag |
| | gcgaacacgtctggactgcggcattcg |
| | gcacctcattactgccgaggcctaccg |
| | gtattaacggtagtggcgctggcaaaa |
| | gaggcggcggtgagacagtctcagcac |
| | gccaattggtaaagtcgctggcgtact |
| | gctggcaacagtagttacctggcggtg |
| | gggccgctattgctacgccgcgtacag |
| | ctaccgatcattgaagtgggcattgcg |
| | ccgctgacgggtgattccgcgctgccg |
| | ctgatatttacagcctggtctatttcg |
| | ctatcgttattctggatcgctctatcc |
| | gggcaagctgctggataccgtgggcaa |
| | cttccagcgccgctgaaaattatcgcg |
| | ctggtcatcctgtctgagccgcaatta |
| | tctggccggcgggttctatcagtacgg |
| | cgactgaggcttatcaaaacgctgcgt |
| | tttctaacggcttcgtcaacggctatc |
| | tgaccatggatacgctgggcgcaatgg |
| | tgtaggtatcgttattgttaacgcggc |
| | gcgactcgtggcgttaccgaagcgcgt |
| | ctgctgacccgttataccgtctgggct |
| | ggcctgatggcgggtgaggtctgactc |
| | tgctgtacctggcgctgaccgtctggg |
| | ttcagacagcgcgtcgctggtcgatca |
| | gtctgcaaacggtgcggcgatcctgca |
| | tgcttacgttcagcatacctaggcggc |
| | ggcggtagatcctgctggcggcgttaa |
| | tcttcatcgcctgcctggtcacggcgg |
| | aggcctgacctgtgatgtgcagaatta |
| | tcgcccagtacgtaccgctctcttatc |
| | gtacgctggtgatatcctcggcggcac |
| | tcgatggtggtgtctaacctcggcttg |
| | agccagctgattcagatctctgtaccg |
| | gtgctgaccgccatttatccgccgtgt |
| | atcgcactggagtattaagattacacg |
| | ctcatggtggcataattcgtcccgcgt |
| | gattgctccgccgatgatatcagcctg |
| | ctattggtattctcgacgggatcaagg |
| | catctgcattcagcgatatcttaccgt |
| | cctgggcgcagcgataccgctggccga |
| | acaaggtctggcgtggttaatgccaac |
| | agtggtgatggtggactggccattatc |
| | tgggatcgtgcggcaggtcgtcaggtg |
| | acctccagcgctcactaatacgcatgg |
| | catggatgaCCGATGGTAGTGTGGGGT |
| | CTCCCCATGCGAGAGTAGGGAACTGCC |
| | AGGCATCAAATAAAACGAAAGGCTCAG |
| | TCGAAAGACTGGGCCTTTCGTTTTATC |
| | TGTTGTTTGTCGGTGAACGCTCTCCTG |
| | AGTAGGACAAATCCGCCGGGAGCGGAT |
| | TTGAACGTTGCGAAGCAACGGCCCGGA |
| | GGGTGGCGGGCAGGACGCCCGCCATAA |
| | ACTGCCAGGCATCAAATTAAGCAGAAG |
| | GCCATCCTGACGGATGGCCTTTTTGCG |
| | TGGCCAGTGCCAAGCTTGCATGCGTGC |
| Ptet-LeuDH- | ttaagacccactacacatttaagttga |
| kivD-yqhD- | tactaatccgcatatgatcaattcaag |
| brnQ | gccgaataagaaggctggctctgcacc |
| construct tet | aggtgatcaaataattcgatagatgtc |
| Repressor is | gtaataatggcggcatactatcagtag |
| in reverse | taggtgatccctttatattagcgactt |
| orientation | gatgctcttgatcaccaatacgcaacc |
| and | taaagtaaaatgccccacagcgctgag |
| underlined; | tgcatataatgcattctctagtgaaaa |
| tet promoter | accttgaggcataaaaaggctaattga |
| with RBS and | ttacgagagatcatactgatactgtag |
| leader region | gccgtgtacctaaatgtacattgctcc |
| is in bold | atcgcgatgacttagtaaagcacatct |
| italics) | aaaactatagcgttattacgtaaaaaa |
| SEQ ID NO: | tcttgccagctttccccttctaaaggg |
| 127 | caaaagtgagtatggtgcctatctaac |
| | atctcaatggctaaggcgtcgagcaaa |
| | gcccgcttattattacatgccaataca |
| | atgtaggctgctctacacctagatctg |
| | ggcgagtttacgggttgttaaaccttc |
| | gattccgacctcattaagcagctctaa |
| | tgcgctgttaatcactttacttttatc |
| | taatctagacat |
| | [symbol] |
| | [symbol] |
| | [symbol] |
| | [symbol] |
| | [symbol] |
| | [symbol] |
| | ATGACTCTTGAAATCTTTGAATATTTA |
| | GAAAAGTACGACTACGAGCAGGTTGTA |
| | TTTTGTCAAGACAAGGAGTCTGGGCTG |
| | AAGGCCATCATTGCCATCCACGACACA |
| | ACCTTAGGCCCGGCGCTTGGCGGAACC |
| | CGCATGTGGACCTACGACTCCGAGGAG |
| | GCGGCCATCGAGGACGCACTTCGTCTT |
| | GCTAAGGGTATGACCTATAAGAACGCG |
| | GCAGCCGGTCTGAATCTGGGGGGTGCT |
| | AAGACTGTAATCATCGGTGATCCACGC |
| | AAGGATAAGAGTGAAGCAATGTTTCGC |
| | GCTTTAGGGCGCTATATTCAGGGCTTG |
| | AACGGCCGCTACATTACCGCAGAAGAC |
| | GTAGGGACAACAGTAGACGACATGGAC |
| | ATCATCCATGAGGAAACTGATTTCGTG |
| | ACCGGTATTTCACCTTCATTCGGGTCA |
| | TCCGGTAACCCTTCCCCCGTAACAGCC |
| | TATGGGGTTTATCGCGGAATGAAGGCC |
| | GCAGCCAAGGAGGCATTTGGCACTGAC |
| | AATTTAGAAGGAAAAGTAATTGCTGTC |
| | CAAGGCGTGGGCAATGTGGCCTACCAT |
| | TTGTGTAAACACCTTCACGCGGAAGGT |
| | GCAAAATTGATCGTTACGGATATTAAC |
| | AAGGAGGCAGTCCAGCGCGCTGTAGAG |
| | GAATTTGGAGCATCGGCTGTGGAACCA |
| | AATGAGATCTACGGTGTAGAATGTGAC |
| | ATTTACGCTCCATGCGCACTTGGTGCC |
| | ACGGTGAATGACGAGACCATCCCCCAA |
| | CTTAAGGCGAAGGTAATCGCTGGTTCA |
| | GCTAACAACCAATTAAAAGAGGACCGT |
| | CACGGAGATATCATCCACGAAATGGGT |
| | ATCGTGTACGCCCCCGATTATGTTATC |
| | AACGCGGGCGGCGTAATCAACGTAGCC |
| | GATGAGCTTTATGGATACAACCGCGAA |
| | CGTGCGCTGAAACGCGTGGAAAGCATT |
| | TATGACACGATCGCAAAGGTAATCGAG |
| | ATCAGTAAGCGCGACGGCATTGCGACA |
| | TACGTGGCAGCGGACCGTCTGGCCGAA |
| | GAACGCATCGCGAGTTTGAAGAATAGC |
| | CGTAGTACCTACTTGCGCAACGGGCAC |
| | GATATTATCAGCCGTCGCtgataagaa |
| | ggagatatacatatgtatacagtagga |
| | GATTACTTATTGGACCGGTTGCACGAA |
| | CTTGGAATTGAGGAAATTTTTGGAGTT |
| | CCGGGTGACTACAACCTGCAGTTCCTT |
| | GACCAAATCATCTCCCATAAGGACATG |
| | AAATGGGTCGGCAATGCCAATGAGCTG |
| | AACGCATCATATATGGCAGACGGGTAT |
| | GCTCGGACCAAAAAGGCTGCAGCATTC |
| | CTTACCACGTTTGGCGTGGGGGAATTA |
| | AGTGCTGTAAATGGACTGGCAGGATCC |
| | TATGCGGAGAATTTACCGGTAGTCGAA |
| | ATTGTTGGCTCGCCTACGTCCAAGGTG |
| | CAGAATGAGGGGAAATTCGTCCATCAC |
| | ACACTTGCAGACGGTGATTTTAAGCAC |
| | TTTATGAAGATGCATGAGCCGGTAACG |
| | GCTGCGCGGACGCTTCTTACTGCGGAA |
| | AACGCAACAGTAGAGATTGATCGCGTT |
| | CTGAGCGCACTGCTTAAGGAACGGAAG |
| | CCCGTCTATATTAACTTACCGGTAGAC |
| | GTGGCCGCAGCCAAAGCCGAAAAACCA |
| | AGCCTGCCTCTTAAGAAGGAGAATTCC |
| | ACGTCCAACACCAGTGACCAAGAGATT |
| | TTGAACAAAATTCAAGAGTCTTTGAAG |
| | AACGCGAAGAAGCCCATCGTAATTACA |
| | GGACATGAGATTATCTCGTTTGGCCTG |
| | GAGAAAACGGTTACACAGTTTATTTCC |
| | AAAACGAAGTTACCTATAACGACGTTA |
| | AACTTTGGAAAGAGCTCTGTGGATGAG |
| | GCACTTCCTAGTTTCTTAGGAATCTAT |
| | AATGGGACCCTTTCAGAGCCAAACTTA |
| | AAGGAATTCGTTGAAAGTGCGGATTTT |
| | ATCTTAATGCTTGGGGTTAAATTGACT |
| | GATTCCAGCACCGGAGCTTTTACGCAC |
| | CATTTAAACGAGAACAAAATGATCTCT |
| | TTGAATATCGACGAAGGCAAAATTTTT |
| | AATGAAAGAATTCAGAACTTTGATTTT |
| | GAATCCCTTATTAGTTCACTTTTAGAT |
| | TTAAGTGAAATAGAGTATAAGGGAAAG |
| | TATATAGACAAGAAGCAAGAGGATTTC |
| | GTTCCGTCTAATGCTCTTTTAAGTCAA |
| | GACAGACTTTGGCAGGCGGTTGAGAAC |
| | CTTACACAATCCAATGAAACGATAGTC |
| | GCCGAACAAGGGACCAGTTTCTTCGGC |
| | GCTTCTTCCATATTCCTGAAGTCTAAG |
| | TCTCATTTCATTGGACAGCCCCTGTGG |
| | GGGTCTATAGGATATACGTTTCCCGCA |
| | GCTCTTGGAAGCCAGATCGCCGATAAG |
| | GAGAGCAGACACCTGTTGTTCATCGGG |
| | GACGGCTCGTTGCAGCTGACTGTTCAG |
| | GAACTGGGGTTGGCGATCAGAGAGAAG |
| | ATTAATCCCATTTGCTTTATCATAAAT |
| | AATGATGGTTATACCGTAGAACGTGAG |
| | ATTCATGGACCTAATCAGAGCTATAAT |
| | GACATTCCTATGTGGAACTATTCAAAA |
| | TTGCCAGAGAGTTTTGGTGCAACTGAG |
| | GATCGCGTTGTTAGTAAAATAGTCCGC |
| | ACGGAGAACGAGTTTGTCAGCGTAATG |
| | AAGGAGGCCCAAGCGGACCCTAATCGG |
| | ATGTACTGGATCGAACTTATTCTGGCT |
| | AAAGAAGGAGCACCTAAAGTTTTAAAG |
| | AAGATGGGAAAACTTTTTgctgaacaa |
| | aataaatcataataagaaggagatata |
| | catatgaacaactttaatctgcacacc |
| | ccaacccgcattctgtaggtaaaggcg |
| | caatcgctggatacgcgaacaaattcc |
| | tcacgatgctcgcgtattgattaccta |
| | cggcggcggcagcgtgaaaaaaaccgg |
| | cgactcgatcaagactggatgccctga |
| | aaggcatggacgtactggaataggcgg |
| | tattgaaccaaacccggcttatgaaac |
| | gctgatgaacgccgtgaaactggacgc |
| | gaacagaaagtgacgacctgctggcgg |
| | ttggcggcggactgtactggacggcac |
| | caaatttatcgccgcagcggctaacta |
| | tccggaaaatatcgatccgtggcacat |
| | tctgcaaacgggcggtaaagagattaa |
| | aagcgccatcccgatgggctgtgtgct |
| | gacgctgccagcaaccggacagaatcc |
| | aacgcaggcgcggtgatctcccgtaaa |
| | accacaggcgacaagcaggcgaccatt |
| | ctgcccatgacagcccgtatttgccgt |
| | gctcgatccggatatacctacaccctg |
| | ccgccgcgtcaggtggctaacggcgta |
| | gtggacgcctagtacacaccgtggaac |
| | agtatgttaccaaaccggagatgccaa |
| | aattcaggaccgatcgcagaaggcatt |
| | agctgacgctgatcgaagatggtccga |
| | aagccctgaaagagccagaaaactacg |
| | atgtgcgcgccaacgtcatgtgggcgg |
| | cgactcaggcgctgaacggatgatcgg |
| | cgctggcgtaccgcaggactgggcaac |
| | gcatatgctgggccacgaactgactgc |
| | gatgcacggtctggatcacgcgcaaac |
| | actggctatcgtcctgcctgcactgtg |
| | gaatgaaaaacgcgataccaagcgcgc |
| | taagctgctgcaatatgctgaacgcgt |
| | ctggaacatcactgaaggacagacgat |
| | gagcgtattgacgccgcgattgccgca |
| | acccgcaatactttgagcaattaggcg |
| | tgctgacccacctctccgactacggtc |
| | tggacggcagctccatcccggctagct |
| | gaaaaaactggaagagcacggcatgac |
| | ccaactgggcgaaaatcatgacattac |
| | gctggatgtcagccgccgtatatacga |
| | agccgcccgctaataagaaggagatat |
| | acatatgacccatcaattaagatcgcg |
| | cgatatcatcgctctgggattatgaca |
| | tttgcgttgacgtcggcgcaggtaaca |
| | ttattaccctccaatggtcggcttgca |
| | ggcaggcgaacacgtctggactgcggc |
| | attcggcttcctcattactgccgttgg |
| | cctaccggtattaacggtagtggcgct |
| | ggcaaaagttggcggcggtgttgacag |
| | tctcagcacgccaattggtaaagtcgc |
| | tggcgtactgctggcaacagtagttac |
| | ctggcggtggggccgctattgctacgc |
| | cgcgtacagctaccgatcattgaagtg |
| | ggcattgcgccgctgacgggtgattcc |
| | gcgctgccgctgatatttacagcctgg |
| | tctatttcgctatcgttattctggttt |
| | cgctctatccgggcaagctgctggata |
| | ccgtgggcaacttccttgcgccgctga |
| | aaattatcgcgctggtcatcctgtctg |
| | agccgcaattatctggccggcgggact |
| | atcagtacggcgactgaggcttatcaa |
| | aacgctgcgttactaacggcttcgtca |
| | acggctatctgaccatggatacgctgg |
| | gcgcaatggtgtaggtatcgttattgt |
| | taacgcggcgcgactcgtggcgttacc |
| | gaagcgcgtctgctgacccgttatacc |
| | gtctgggctggcctgatggcgggtgag |
| | gtctgactctgctgtacctggcgctga |
| | ccgtctgggttcagacagcgcgtcgct |
| | ggtcgatcagtctgcaaacggtgcggc |
| | gatcctgcatgcttacgttcagcatac |
| | ctaggcggcggcggtagatcctgctgg |
| | cggcgttaatcttcatcgcctgcctgg |
| | tcacggcggttggcctgacctgtgctt |
| | gtgcagaattcttcgcccagtacgtac |
| | cgctctcttatcgtacgctggtgttta |
| | tcctcggcggcttctcgatggtggtgt |
| | ctaacctcggcttgagccagctgattc |
| | agatctctgtaccggtgctgaccgcca |
| | tttatccgccgtgtatcgcactggagt |
| | attaagattacacgctcatggtggcat |
| | aattcgtcccgcgtgattgctccgccg |
| | atgatatcagcctgctattggtattct |
| | cgacgggatcaaggcatctgcattcag |
| | cgatatcttaccgtcctgggcgcagcg |
| | ataccgctggccgaacaaggtctggcg |
| | tggttaatgccaacagtggtgatggtg |
| | gactggccattatctgggatcgtgcgg |
| | caggtcgtcaggtgacctccagcgctc |
| | actaatacgcatggcatggatgaCCGA |
| | TGGTAGTGTGGGGTCTCCCCATGCGAG |
| | AGTAGGGAACTGCCAGGCATCAAATAA |
| | AACGAAAGGCTCAGTCGAAAGACTGGG |
| | CCTTTCGTTTTATCTGTTGTTTGTCGG |
| | TGAACGCTCTCCTGAGTAGGACAAATC |
| | CGCCGGGAGCGGATTTGAACGTTGCGA |
| | AGCAACGGCCCGGAGGGTGGCGGGCAG |
| | GACGCCCGCCATAAACTGCCAGGCATC |
| | AAATTAAGCAGAAGGCCATCCTGACGG |
| | ATGGCCTTTTTGCGTGGCCAGTGCCAA |
| | GCTTGCATGCGTGC |
| LeuDH-kivD- | ATGACTCTTGAAATCTTTGAATATTTA |
| yqhD-brnQ | GAAAAGTACGACTACGAGCAGGTTGTA |
| construct | TTTTGTCAAGACAAGGAGTCTGGGCTG |
| (RBS are | AAGGCCATCATTGCCATCCACGACACA |
| underlined) | ACCTTAGGCCCGGCGCTTGGCGGAACC |
| SEQ ID NO: | CGCATGTGGACCTACGACTCCGAGGAG |
| 128 | GCGGCCATCGAGGACGCACTTCGTCTT |
| | GCTAAGGGTATGACCTATAAGAACGCG |
| | GCAGCCGGTCTGAATCTGGGGGGTGCT |
| | AAGACTGTAATCATCGGTGATCCACGC |
| | AAGGATAAGAGTGAAGCAATGTTTCGC |
| | GCTTTAGGGCGCTATATTCAGGGCTTG |
| | AACGGCCGCTACATTACCGCAGAAGAC |
| | GTAGGGACAACAGTAGACGACATGGAC |
| | ATCATCCATGAGGAAACTGATTTCGTG |
| | ACCGGTATTTCACCTTCATTCGGGTCA |
| | TCCGGTAACCCTTCCCCCGTAACAGCC |
| | TATGGGGTTTATCGCGGAATGAAGGCC |
| | GCAGCCAAGGAGGCATTTGGCACTGAC |
| | AATTTAGAAGGAAAAGTAATTGCTGTC |
| | CAAGGCGTGGGCAATGTGGCCTACCAT |
| | TTGTGTAAACACCTTCACGCGGAAGGT |
| | GCAAAATTGATCGTTACGGATATTAAC |
| | AAGGAGGCAGTCCAGCGCGCTGTAGAG |
| | GAATTTGGAGCATCGGCTGTGGAACCA |
| | AATGAGATCTACGGTGTAGAATGTGAC |
| | ATTTACGCTCCATGCGCACTTGGTGCC |
| | ACGGTGAATGACGAGACCATCCCCCAA |
| | CTTAAGGCGAAGGTAATCGCTGGTTCA |
| | GCTAACAACCAATTAAAAGAGGACCGT |
| | CACGGAGATATCATCCACGAAATGGGT |
| | ATCGTGTACGCCCCCGATTATGTTATC |
| | AACGCGGGCGGCGTAATCAACGTAGCC |
| | GATGAGCTTTATGGATACAACCGCGAA |
| | CGTGCGCTGAAACGCGTGGAAAGCATT |
| | TATGACACGATCGCAAAGGTAATCGAG |
| | ATCAGTAAGCGCGACGGCATTGCGACA |
| | TACGTGGCAGCGGACCGTCTGGCCGAA |
| | GAACGCATCGCGAGTTTGAAGAATAGC |
| | CGTAGTACCTACTTGCGCAACGGGCAC |
| | GATATTATCAGCCGTCGCtgataagaa |
| | ggagatatacatatgtatacagtagga |
| | GATTACTTATTGGACCGGTTGCACGAA |
| | CTTGGAATTGAGGAAATTTTTGGAGTT |
| | CCGGGTGACTACAACCTGCAGTTCCTT |
| | GACCAAATCATCTCCCATAAGGACATG |
| | AAATGGGTCGGCAATGCCAATGAGCTG |
| | AACGCATCATATATGGCAGACGGGTAT |
| | GCTCGGACCAAAAAGGCTGCAGCATTC |
| | CTTACCACGTTTGGCGTGGGGGAATTA |
| | AGTGCTGTAAATGGACTGGCAGGATCC |
| | TATGCGGAGAATTTACCGGTAGTCGAA |
| | ATTGTTGGCTCGCCTACGTCCAAGGTG |
| | CAGAATGAGGGGAAATTCGTCCATCAC |
| | ACACTTGCAGACGGTGATTTTAAGCAC |
| | TTTATGAAGATGCATGAGCCGGTAACG |
| | GCTGCGCGGACGCTTCTTACTGCGGAA |
| | AACGCAACAGTAGAGATTGATCGCGTT |
| | CTGAGCGCACTGCTTAAGGAACGGAAG |
| | CCCGTCTATATTAACTTACCGGTAGAC |
| | GTGGCCGCAGCCAAAGCCGAAAAACCA |
| | AGCCTGCCTCTTAAGAAGGAGAATTCC |
| | ACGTCCAACACCAGTGACCAAGAGATT |
| | TTGAACAAAATTCAAGAGTCTTTGAAG |
| | AACGCGAAGAAGCCCATCGTAATTACA |
| | GGACATGAGATTATCTCGTTTGGCCTG |
| | GAGAAAACGGTTACACAGTTTATTTCC |
| | AAAACGAAGTTACCTATAACGACGTTA |
| | AACTTTGGAAAGAGCTCTGTGGATGAG |
| | GCACTTCCTAGTTTCTTAGGAATCTAT |
| | AATGGGACCCTTTCAGAGCCAAACTTA |
| | AAGGAATTCGTTGAAAGTGCGGATTTT |
| | ATCTTAATGCTTGGGGTTAAATTGACT |
| | GATTCCAGCACCGGAGCTTTTACGCAC |
| | CATTTAAACGAGAACAAAATGATCTCT |
| | TTGAATATCGACGAAGGCAAAATTTTT |
| | AATGAAAGAATTCAGAACTTTGATTTT |
| | GAATCCCTTATTAGTTCACTTTTAGAT |
| | TTAAGTGAAATAGAGTATAAGGGAAAG |
| | TATATAGACAAGAAGCAAGAGGATTTC |
| | GTTCCGTCTAATGCTCTTTTAAGTCAA |
| | GACAGACTTTGGCAGGCGGTTGAGAAC |
| | CTTACACAATCCAATGAAACGATAGTC |
| | GCCGAACAAGGGACCAGTTTCTTCGGC |
| | GCTTCTTCCATATTCCTGAAGTCTAAG |
| | TCTCATTTCATTGGACAGCCCCTGTGG |
| | GGGTCTATAGGATATACGTTTCCCGCA |
| | GCTCTTGGAAGCCAGATCGCCGATAAG |
| | GAGAGCAGACACCTGTTGTTCATCGGG |
| | GACGGCTCGTTGCAGCTGACTGTTCAG |
| | GAACTGGGGTTGGCGATCAGAGAGAAG |
| | ATTAATCCCATTTGCTTTATCATAAAT |
| | AATGATGGTTATACCGTAGAACGTGAG |
| | ATTCATGGACCTAATCAGAGCTATAAT |
| | GACATTCCTATGTGGAACTATTCAAAA |
| | TTGCCAGAGAGTTTTGGTGCAACTGAG |
| | GATCGCGTTGTTAGTAAAATAGTCCGC |
| | ACGGAGAACGAGTTTGTCAGCGTAATG |
| | AAGGAGGCCCAAGCGGACCCTAATCGG |
| | ATGTACTGGATCGAACTTATTCTGGCT |
| | AAAGAAGGAGCACCTAAAGTTTTAAAG |
| | AAGATGGGAAAACTTTTTgctgaacaa |
| | aataaatcataataagaaggagatata |
| | catatgaacaactttaatctgcacacc |
| | ccaacccgcattctgtaggtaaaggcg |
| | caatcgctggatacgcgaacaaattcc |
| | tcacgatgctcgcgtattgattaccta |
| | cggcggcggcagcgtgaaaaaaaccgg |
| | cgactcgatcaagactggatgccctga |
| | aaggcatggacgtactggaataggcgg |
| | tattgaaccaaacccggcttatgaaac |
| | gctgatgaacgccgtgaaactggacgc |
| | gaacagaaagtgacgacctgctggcgg |
| | aggcggcggactgtactggacggcacc |
| | aaatttatcgccgcagcggctaactat |
| | ccggaaaatatcgatccgtggcacatt |
| | ctgcaaacgggcggtaaagagattaaa |
| | agcgccatcccgatgggctgtgtgctg |
| | acgctgccagcaaccggttcagaatcc |
| | aacgcaggcgcggtgatctcccgtaaa |
| | accacaggcgacaagcaggcgaccatt |
| | ctgcccatgacagcccgtatttgccgt |
| | gctcgatccggatatacctacaccctg |
| | ccgccgcgtcaggtggctaacggcgta |
| | gtggacgcctagtacacaccgtggaac |
| | agtatgttaccaaaccggagatgccaa |
| | aattcaggaccgatcgcagaaggcatt |
| | agctgacgctgatcgaagatggtccga |
| | aagccctgaaagagccagaaaactacg |
| | atgtgcgcgccaacgtcatgtgggcgg |
| | cgactcaggcgctgaacggtttgatcg |
| | gcgctggcgtaccgcaggactgggcaa |
| | cgcatatgctgggccacgaactgactg |
| | cgatgcacggtctggatcacgcgcaaa |
| | cactggctatcgtcctgcctgcactgt |
| | ggaatgaaaaacgcgataccaagcgcg |
| | ctaagctgctgcaatatgctgaacgcg |
| | tctggaacatcactgaaggacagacga |
| | tgagcgtattgacgccgcgattgccgc |
| | aacccgcaatactagagcaattaggcg |
| | tgctgacccacctctccgactacggtc |
| | tggacggcagctccatcccggctagct |
| | gaaaaaactggaagagcacggcatgac |
| | ccaactgggcgaaaatcatgacattac |
| | gctggatgtcagccgccgtatatacga |
| | agccgcccgctaataagaaggagatat |
| | acatatgacccatcaattaagatcgcg |
| | cgatatcatcgctctgggattatgaca |
| | tttgcgttgacgtcggcgcaggtaaca |
| | ttattaccctccaatggtcggcttgca |
| | ggcaggcgaacacgtctggactgcggc |
| | attcggcacctcattactgccgaggcc |
| | taccggtattaacggtagtggcgctgg |
| | caaaagaggcggcggtgagacagtctc |
| | agcacgccaattggtaaagtcgctggc |
| | gtactgctggcaacagtagttacctgg |
| | cggtggggccgctattgctacgccgcg |
| | tacagctaccgatcattgaagtgggca |
| | ttgcgccgctgacgggtgattccgcgc |
| | tgccgctgatatttacagcctggtcta |
| | tacgctatcgttattctggatcgctct |
| | atccgggcaagctgctggataccgtgg |
| | gcaacttccttgcgccgctgaaaatta |
| | tcgcgctggtcatcctgtctgagccgc |
| | aattatctggccggcgggactatcagt |
| | acggcgactgaggcttatcaaaacgct |
| | gcgttactaacggcttcgtcaacggct |
| | atctgaccatggatacgctgggcgcaa |
| | tggtgtaggtatcgttattgttaacgc |
| | ggcgcgactcgtggcgttaccgaagcg |
| | cgtctgctgacccgttataccgtctgg |
| | gctggcctgatggcgggtgaggtctga |
| | ctctgctgtacctggcgctgaccgtct |
| | gggttcagacagcgcgtcgctggtcga |
| | tcagtctgcaaacggtgcggcgatcct |
| | gcatgcttacgttcagcatacctaggc |
| | ggcggcggtagcttcctgctggcggcg |
| | ttaatcttcatcgcctgcctggtcacg |
| | gcggttggcctgacctgtgcttgtgca |
| | gaattatcgcccagtacgtaccgctct |
| | cttatcgtacgctggtgatatcctcgg |
| | cggcactcgatggtggtgtctaacctc |
| | ggcttgagccagctgattcagatctct |
| | gtaccggtgctgaccgccatttatccg |
| | ccgtgtatcgcactggagtattaagat |
| | tacacgctcatggtggcataattcgtc |
| | ccgcgtgattgctccgccgatgatatc |
| | agcctgctttttggtattctcgacggg |
| | atcaaggcatctgcattcagcgatatc |
| | ttaccgtcctgggcgcagcgtttaccg |
| | ctggccgaacaaggtctggcgtggtta |
| | atgccaacagtggtgatggtggactgg |
| | ccattatctgggatcgtgcggcaggtc |
| | gtcaggtgacctccagcgctcactaa |
| Pfnrs-LeuDH- | AGTTGTTCTTATTGGTGGTGTTGCTTT |
| kivD-yqhD- | ATGGTTGCATCGTAGTAAATGGTTGTA |
| brnQ | ACAAAAGCAATTTTTCCGGCTGTCTGT |
| construct | ATACAAAAACGCCGCAAAGTTTGAGCG |
| (RBS are | AAGTCAATAAACTCTCTACCCATTCAG |
| underlined); | GGCAATATCTCTCTTggatccaaagtg |
| FNR | aactctagaaataattagataacttta |
| promoter with | agaaggagatatacatATGACTCTTGA |
| RBS and | AATCTTTGAATATTTAGAAAAGTACGA |
| leader region | CTACGAGCAGGTTGTATTTTGTCAAGA |
| (underlined), | CAAGGAGTCTGGGCTGAAGGCCATCAT |
| FNR binding | TGCCATCCACGACACAACCTTAGGCCC |
| site bold | GGCGCTTGGCGGAACCCGCATGTGGAC |
| SEQ ID NO: | CTACGACTCCGAGGAGGCGGCCATCGA |
| 129 | GGACGCACTTCGTCTTGCTAAGGGTAT |
| | GACCTATAAGAACGCGGCAGCCGGTCT |
| | GAATCTGGGGGGTGCTAAGACTGTAAT |
| | CATCGGTGATCCACGCAAGGATAAGAG |
| | TGAAGCAATGTTTCGCGCTTTAGGGCG |
| | CTATATTCAGGGCTTGAACGGCCGCTA |
| | CATTACCGCAGAAGACGTAGGGACAAC |
| | AGTAGACGACATGGACATCATCCATGA |
| | GGAAACTGATTTCGTGACCGGTATTTC |
| | ACCTTCATTCGGGTCATCCGGTAACCC |
| | TTCCCCCGTAACAGCCTATGGGGTTTA |
| | TCGCGGAATGAAGGCCGCAGCCAAGGA |
| | GGCATTTGGCACTGACAATTTAGAAGG |
| | AAAAGTAATTGCTGTCCAAGGCGTGGG |
| | CAATGTGGCCTACCATTTGTGTAAACA |
| | CCTTCACGCGGAAGGTGCAAAATTGAT |
| | CGTTACGGATATTAACAAGGAGGCAGT |
| | CCAGCGCGCTGTAGAGGAATTTGGAGC |
| | ATCGGCTGTGGAACCAAATGAGATCTA |
| | CGGTGTAGAATGTGACATTTACGCTCC |
| | ATGCGCACTTGGTGCCACGGTGAATGA |
| | CGAGACCATCCCCCAACTTAAGGCGAA |
| | GGTAATCGCTGGTTCAGCTAACAACCA |
| | ATTAAAAGAGGACCGTCACGGAGATAT |
| | CATCCACGAAATGGGTATCGTGTACGC |
| | CCCCGATTATGTTATCAACGCGGGCGG |
| | CGTAATCAACGTAGCCGATGAGCTTTA |
| | TGGATACAACCGCGAACGTGCGCTGAA |
| | ACGCGTGGAAAGCATTTATGACACGAT |
| | CGCAAAGGTAATCGAGATCAGTAAGCG |
| | CGACGGCATTGCGACATACGTGGCAGC |
| | GGACCGTCTGGCCGAAGAACGCATCGC |
| | GAGTTTGAAGAATAGCCGTAGTACCTA |
| | CTTGCGCAACGGGCACGATATTATCAG |
| | CCGTCGCtgataagaaggagatataca |
| | tatgtatacagtaggaGATTACTTATT |
| | GGACCGGTTGCACGAACTTGGAATTGA |
| | GGAAATTTTTGGAGTTCCGGGTGACTA |
| | CAACCTGCAGTTCCTTGACCAAATCAT |
| | CTCCCATAAGGACATGAAATGGGTCGG |
| | CAATGCCAATGAGCTGAACGCATCATA |
| | TATGGCAGACGGGTATGCTCGGACCAA |
| | AAAGGCTGCAGCATTCCTTACCACGTT |
| | TGGCGTGGGGGAATTAAGTGCTGTAAA |
| | TGGACTGGCAGGATCCTATGCGGAGAA |
| | TTTACCGGTAGTCGAAATTGTTGGCTC |
| | GCCTACGTCCAAGGTGCAGAATGAGGG |
| | GAAATTCGTCCATCACACACTTGCAGA |
| | CGGTGATTTTAAGCACTTTATGAAGAT |
| | GCATGAGCCGGTAACGGCTGCGCGGAC |
| | GCTTCTTACTGCGGAAAACGCAACAGT |
| | AGAGATTGATCGCGTTCTGAGCGCACT |
| | GCTTAAGGAACGGAAGCCCGTCTATAT |
| | TAACTTACCGGTAGACGTGGCCGCAGC |
| | CAAAGCCGAAAAACCAAGCCTGCCTCT |
| | TAAGAAGGAGAATTCCACGTCCAACAC |
| | CAGTGACCAAGAGATTTTGAACAAAAT |
| | TCAAGAGTCTTTGAAGAACGCGAAGAA |
| | GCCCATCGTAATTACAGGACATGAGAT |
| | TATCTCGTTTGGCCTGGAGAAAACGGT |
| | TACACAGTTTATTTCCAAAACGAAGTT |
| | ACCTATAACGACGTTAAACTTTGGAAA |
| | GAGCTCTGTGGATGAGGCACTTCCTAG |
| | TTTCTTAGGAATCTATAATGGGACCCT |
| | TTCAGAGCCAAACTTAAAGGAATTCGT |
| | TGAAAGTGCGGATTTTATCTTAATGCT |
| | TGGGGTTAAATTGACTGATTCCAGCAC |
| | CGGAGCTTTTACGCACCATTTAAACGA |
| | GAACAAAATGATCTCTTTGAATATCGA |
| | CGAAGGCAAAATTTTTAATGAAAGAAT |
| | TCAGAACTTTGATTTTGAATCCCTTAT |
| | TAGTTCACTTTTAGATTTAAGTGAAAT |
| | AGAGTATAAGGGAAAGTATATAGACAA |
| | GAAGCAAGAGGATTTCGTTCCGTCTAA |
| | TGCTCTTTTAAGTCAAGACAGACTTTG |
| | GCAGGCGGTTGAGAACCTTACACAATC |
| | CAATGAAACGATAGTCGCCGAACAAGG |
| | GACCAGTTTCTTCGGCGCTTCTTCCAT |
| | ATTCCTGAAGTCTAAGTCTCATTTCAT |
| | TGGACAGCCCCTGTGGGGGTCTATAGG |
| | ATATACGTTTCCCGCAGCTCTTGGAAG |
| | CCAGATCGCCGATAAGGAGAGCAGACA |
| | CCTGTTGTTCATCGGGGACGGCTCGTT |
| | GCAGCTGACTGTTCAGGAACTGGGGTT |
| | GGCGATCAGAGAGAAGATTAATCCCAT |
| | TTGCTTTATCATAAATAATGATGGTTA |
| | TACCGTAGAACGTGAGATTCATGGACC |
| | TAATCAGAGCTATAATGACATTCCTAT |
| | GTGGAACTATTCAAAATTGCCAGAGAG |
| | TTTTGGTGCAACTGAGGATCGCGTTGT |
| | TAGTAAAATAGTCCGCACGGAGAACGA |
| | GTTTGTCAGCGTAATGAAGGAGGCCCA |
| | AGCGGACCCTAATCGGATGTACTGGAT |
| | CGAACTTATTCTGGCTAAAGAAGGAGC |
| | ACCTAAAGTTTTAAAGAAGATGGGAAA |
| | ACTTTTTgctgaacaaaataaatcata |
| | ataagaaggagatatacatatgaacaa |
| | ctttaatctgcacaccccaacccgcat |
| | tctgtaggtaaaggcgcaatcgctgga |
| | tacgcgaacaaattcctcacgatgctc |
| | gcgtattgattacctacggcggcggca |
| | gcgtgaaaaaaaccggcgactcgatca |
| | agactggatgccctgaaaggcatggac |
| | gtactggaataggcggtattgaaccaa |
| | acccggcttatgaaacgctgatgaacg |
| | ccgtgaaactggacgcgaacagaaagt |
| | gacgacctgctggcggaggcggcggac |
| | tgtactggacggcaccaaatttatcgc |
| | cgcagcggctaactatccggaaaatat |
| | cgatccgtggcacattctgcaaacggg |
| | cggtaaagagattaaaagcgccatccc |
| | gatgggctgtgtgctgacgctgccagc |
| | aaccggttcagaatccaacgcaggcgc |
| | ggtgatctcccgtaaaaccacaggcga |
| | caagcaggcgaccattctgcccatgac |
| | agcccgtatttgccgtgctcgatccgg |
| | atatacctacaccctgccgccgcgtca |
| | ggtggctaacggcgtagtggacgccta |
| | gtacacaccgtggaacagtatgttacc |
| | aaaccggagatgccaaaattcaggacc |
| | gatcgcagaaggcattagctgacgctg |
| | atcgaagatggtccgaaagccctgaaa |
| | gagccagaaaactacgatgtgcgcgcc |
| | aacgtcatgtgggcggcgactcaggcg |
| | ctgaacggtttgatcggcgctggcgta |
| | ccgcaggactgggcaacgcatatgctg |
| | ggccacgaactgactgcgatgcacggt |
| | ctggatcacgcgcaaacactggctatc |
| | gtcctgcctgcactgtggaatgaaaaa |
| | cgcgataccaagcgcgctaagctgctg |
| | caatatgctgaacgcgtctggaacatc |
| | actgaaggacagacgatgagcgtattg |
| | acgccgcgattgccgcaacccgcaata |
| | ctagagcaattaggcgtgctgacccac |
| | ctctccgactacggtctggacggcagc |
| | tccatcccggctagctgaaaaaactgg |
| | aagagcacggcatgacccaactgggcg |
| | aaaatcatgacattacgctggatgtca |
| | gccgccgtatatacgaagccgcccgct |
| | aataagaaggagatatacatatgaccc |
| | atcaattaagatcgcgcgatatcatcg |
| | ctctgggattatgacatttgcgttgac |
| | gtcggcgcaggtaacattattaccctc |
| | caatggtcggcttgcaggcaggcgaac |
| | acgtctggactgcggcattcggcacct |
| | cattactgccgaggcctaccggtatta |
| | acggtagtggcgctggcaaaagaggcg |
| | gcggtgagacagtctcagcacgccaat |
| | tggtaaagtcgctggcgtactgctggc |
| | aacagtagttacctggcggtggggccg |
| | ctattgctacgccgcgtacagctaccg |
| | atcattgaagtgggcattgcgccgctg |
| | acgggtgattccgcgctgccgctgata |
| | tttacagcctggtctatacgctatcgt |
| | tattctggatcgctctatccgggcaag |
| | ctgctggataccgtgggcaacttcctt |
| | gcgccgctgaaaattatcgcgctggtc |
| | atcctgtctgagccgcaattatctggc |
| | cggcgggactatcagtacggcgactga |
| | ggcttatcaaaacgctgcgttactaac |
| | ggcttcgtcaacggctatctgaccatg |
| | gatacgctgggcgcaatggtgtaggta |
| | tcgttattgttaacgcggcgcgactcg |
| | tggcgttaccgaagcgcgtctgctgac |
| | ccgttataccgtctgggctggcctgat |
| | ggcgggtgaggtctgactctgctgtac |
| | ctggcgctgaccgtctgggttcagaca |
| | gcgcgtcgctggtcgatcagtctgcaa |
| | acggtgcggcgatcctgcatgcttacg |
| | ttcagcatacctaggcggcggcggtag |
| | cttcctgctggcggcgttaatcttcat |
| | cgcctgcctggtcacggcggttggcct |
| | gacctgtgcttgtgcagaattatcgcc |
| | cagtacgtaccgctctcttatcgtacg |
| | ctggtgatatcctcggcggcactcgat |
| | ggtggtgtctaacctcggcttgagcca |
| | gctgattcagatctctgtaccggtgct |
| | gaccgccatttatccgccgtgtatcgc |
| | actggagtattaagattacacgctcat |
| | ggtggcataattcgtcccgcgtgattg |
| | ctccgccgatgatatcagcctgctttt |
| | tggtattctcgacgggatcaaggcatc |
| | tgcattcagcgatatcttaccgtcctg |
| | ggcgcagcgtttaccgctggccgaaca |
| | aggtctggcgtggttaatgccaacagt |
| | ggtgatggtggactggccattatctgg |
| | gatcgtgcggcaggtcgtcaggtgacc |
| | tccagcgctcactaatacgcatggcat |
| | ggatgaCCGATGGTAGTGTGGGGTCTC |
| | CCCATGCGAGAGTAGGGAACTGCCAGG |
| | CATCAAATAAAACGAAAGGCTCAGTCG |
| | AAAGACTGGGCCTTTCGTTTTATCTGT |
| | TGTTTGTCGGTGAACGCTCTCCTGAGT |
| | AGGACAAATCCGCCGGGAGCGGATTTG |
| | AACGTTGCGAAGCAACGGCCCGGAGGG |
| | TGGCGGGCAGGACGCCCGCCATAAACT |
| | GCCAGGCATCAAATTAAGCAGAAGGCC |
| | ATCCTGACGGATGGCCTTTTTGCGTGG |
| | CCAGTGCCAAGCTTGCATGCGTGC |

TABLE 26
Primer Sequences Example 27

| Name | Sequence | SEQ ID NO |
|---|---|---|
| SR36 Primer | tagaactgatgcaaaaagtgctcgacgaaggcacacagaTGTGTAG | SEQ ID NO: 130 |
| | GCTGGAGCTGCTTC | |
| SR38 Primer | gtttcgtaattagatagccaccggcgctttaatgcccggaCATATGAA | SEQ ID NO: 131 |
| | TATCCTCCTTAG | |
| SR33 Primer | caacacgtacctgaggaaccatgaaacagtatttagaactgatgcaaaaag | SEQ ID NO: 132 |
| SR34 Primer | cgcacactggcgtcggctctggcaggatgatcgtaattagatagc | SEQ ID NO: 133 |
| SR43 Primer | atatcgtcgcagcccacagcaacacgtttcctgagg | SEQ ID NO: 134 |
| SR44 Primer | aagaatttaacggagggcaaaaaaaaccgacgcacactggcgtcggc | SEQ ID NO: 135 |

1. A bacterium comprising gene sequence(s) encoding one or more branched chain amino acid catabolism enzyme(s) operably linked to a directly or indirectly inducible promoter that is not associated with the branched chain amino acid catabolism enzyme gene in nature.

2. The bacterium of claim 1, wherein the bacterium further comprises gene sequence(s) encoding one or more transporter(s) of a branched chain amino acid operably linked to a promoter that is not associated with the transporter gene in nature.

3. The bacterium of claim 2, wherein the promoter is a directly or indirectly inducible promoter.

4. The bacterium of claim 1 or claim 2, wherein the bacterium further comprises a genetic modification that reduces export of a branched chain amino acid from the bacterium.

5. The bacterium of claim 1 or claim 2, wherein the bacterium further comprises a genetic modification that reduces endogenous biosynthesis of a branched chain amino acid in the bacterium.

6. The bacterium of claim 1 or claim 2, wherein the bacterium further comprises gene sequence(s) encoding one or more branched chain amino acid binding protein(s).

7. The bacterium of claim 2, wherein the promoter operably linked to the gene sequence(s) encoding a branched chain amino acid catabolism enzyme and the promoter operably linked to the gene sequence(s) encoding a transporter of a branched chain amino acid are separate copies of the same promoter.

8. The bacterium of claim 2, wherein the promoter operably linked to the gene sequence(s) encoding a branched chain amino acid catabolism enzyme and the promoter operably linked to the gene sequence(s) encoding a transporter of a branched chain amino acid are the same copy of the same promoter.

9. The bacterium of claim 2, wherein the promoter operably linked to the gene sequence(s) encoding a branched chain amino acid catabolism enzyme and the promoter operably linked to the gene sequence(s) encoding a transporter of a branched chain amino acid are different promoters.

10. The bacterium of claim 1, wherein the promoter operably linked to the gene sequence(s) encoding a branched chain amino acid catabolism enzyme is directly or indirectly induced by exogenous environmental conditions found in the mammalian gut.

11. The bacterium of claim 1 or claim 2, wherein the promoter operably linked to the gene sequence(s) encoding a branched chain amino acid catabolism enzyme is directly or indirectly induced under low-oxygen or anaerobic conditions.

12. The bacterium of claim 11, wherein the promoter operably linked to the gene sequence(s) encoding a branched chain amino acid catabolism enzyme is selected from the group consisting of an FNR-responsive promoter, an ANR-responsive promoter, and a DNR-responsive promoter.

13. The bacterium of claim 12, wherein the promoter operably linked to the gene sequence(s) encoding a branched chain amino acid catabolism enzyme is an FNRS promoter.

14. The bacterium of claim 2, wherein the promoter operably linked to the gene sequence(s) encoding a transporter of a branched chain amino acid is directly or indirectly induced by exogenous environmental conditions found in the mammalian gut.

15. The bacterium of claim 2, wherein the promoter operably linked to the gene sequence(s) encoding a transporter of a branched chain amino acid is directly or indirectly induced under low-oxygen or anaerobic conditions.

16. The bacterium of claim 15, wherein the promoter operably linked to the gene sequence(s) encoding a transporter of a branched chain amino acid is selected from the group consisting of an FNR-responsive promoter, an ANR-responsive promoter, and a DNR-responsive promoter.

17. The bacterium of claim 16, wherein the promoter operably linked to the gene sequence(s) encoding a transporter of a branched chain amino acid catabolism enzyme is an FNRS promoter.

18. The bacterium of claim 1, wherein the gene sequence(s) encoding a branched chain amino acid catabolism enzyme is located on a chromosome in the bacterium.

19. The bacterium of claim 1, wherein the gene sequence(s) encoding a branched chain amino acid catabolism enzyme is located on a plasmid in the bacterium.

20. The bacterium of claim 1, wherein at least one gene sequence(s) encoding a branched chain amino acid catabolism enzyme is located on a plasmid in the bacterium and at least one gene sequence(s) encoding a branched chain amino acid catabolism enzyme is located on a chromosome in the bacterium.

21. The bacterium of claim 2, wherein the gene sequence(s) encoding a transporter of a branched chain amino acid is located on a chromosome in the bacterium.

22. The bacterium of claim 2, wherein the gene sequence(s) encoding a transporter of a branched chain amino acid is located on a plasmid in the bacterium.

23. The bacterium of claim 2, wherein at least one gene sequence(s) encoding a transporter of a branched chain amino acid is located on a plasmid in the bacterium and at least one gene sequence(s) encoding a transporter of a branched chain amino acid is located on a chromosome in the bacterium.

24. The bacterium of claim 1, wherein the branched chain amino acid is leucine, isoleucine, or valine.

25. The bacterium of claim 24, wherein the branched chain amino acid is leucine.

26. The bacterium of claim 1, wherein the bacterium comprises gene sequence(s) encoding one or more branched chain amino acid catabolism enzyme(s) that are capable of converting α-ketoisocaproate, α-keto-β-methylvalerate, and/or α-ketoisovalerate to isovaleraldehyde, 2-methylbutyraldehyde, and/or isobutyraldehyde, respectively.

27. The bacterium of claim 1, wherein the bacterium comprises gene sequence(s) encoding one or more α-ketoacid decarboxylase(s).

28. The bacterium of claim 27, wherein the gene sequence(s) encodes kivD.

29. The bacterium of claim 28, wherein the kivD gene is a Lactococcus lactis kivD gene.

30. The bacterium of claim 1, wherein the bacterium comprises gene sequence(s) encoding one or more branched chain amino acid catabolism enzyme(s) that are capable of converting leucine, isoleucine and/or valine to α-ketoisocaproate, α-keto-β-methylvalerate, and/or α-ketoisovalerate, respectively.

31. The bacterium of claim 1, wherein the bacterium comprises gene sequence(s) encoding one or more branched chain amino acid deamination enzymes.

32. The bacterium of claim 31, wherein the bacterium comprises gene sequence(s) encoding a gene selected from a branched chain amino acid dehydrogenase, branched chain amino acid aminotransferase, and amino acid oxidase.

33. The bacterium of claim 32, wherein the branched chain amino acid dehydrogenase is leucine dehydrogenase.

34. The bacterium of claim 33, wherein the leucine dehydrogenase is a Bacillus cereus leucine dehydrogenase.

35. The bacterium of claim 32, wherein the branched chain amino acid aminotransferase is ilvE.

36. The bacterium of claim 32, wherein amino acid oxidase is L-AAD.

37. The bacterium of claim 36, wherein the L-AAD gene is a proteus vulgaris L-AAD gene or a proteus mirabilis L-AAD gene.

38. The bacterium of claim 1, wherein the bacterium comprises gene sequence(s) encoding one or more branched chain amino acid catabolism enzyme(s) that are capable of converting isovaleraldehyde, isobutyraldehyde and/or 2-methylbutyraldehyde to isopentanol, isobutanol, and/or 2-methybutanol, respectively.

39. The bacterium of claim 1, wherein the bacterium comprises gene sequence(s) encoding one or more branched chain amino acid alcohol dehydrogenases.

40. The bacterium of claim 39, wherein the branched chain amino acid alcohol dehydrogenase gene is selected from adh1, adh2, adh2, adh4, adh5, adh6, adh7, sfa1, and yqhD.

41. The bacterium of claim 40, wherein the branched chain amino acid alcohol dehydrogenase gene is adh2.

42. The bacterium of claim 41, wherein the adh2 is a S. cerevisiae adh2.

43. The bacterium of claim 40, wherein the branched chain amino acid alcohol dehydrogenase gene is yqhD.

44. The bacterium of claim 43, wherein the yqhD gene is a E. coli yqhD gene.

45. The bacterium of claim 1, wherein the bacterium comprises gene sequence(s) encoding one or more branched chain amino acid catabolism enzyme(s) that are capable of converting isovaleraldehyde, isobutyraldehyde and/or 2-methylbutyraldehyde to isovalerate, isobutyrate, and/or 2-methybutyrate, respectively.

46. The bacterium of claim 1, wherein the bacterium comprises gene sequence(s) encoding one or more branched chain amino acid aldehyde dehydrogenases.

47. The bacterium of claim 46, wherein the branched chain amino acid aldehyde dehydrogenase gene is padA.

48. The bacterium of claim 47, wherein the padA is an E. coli padA.

49. The bacterium of claim 2, wherein the bacterium comprises gene sequence(s) encoding one or more transporters of branched chain amino acid selected from livKHMGF and brnQ.

50. The bacterium of claim 49, wherein the livKHMGF is an E. coli livKHMGF.

51. The bacterium of claim 4, wherein the genetic modification that reduces export of a branched chain amino acid from the bacterium is gene modification in the leuE gene.

52. The bacterium of claim 51, wherein the leuE gene is deleted from the bacterium.

53. The bacterium of claim 5, wherein the genetic modification that reduces endogenous biosynthesis of a branched chain amino acid in the bacterium is a gene modification in the ilvC gene.

54. The bacterium of claim 53, wherein the ilvC gene is deleted from the bacterium.

55. The bacterium of claim 6, wherein the bacterium further comprises gene sequence(s) encoding ilvJ.

56. A bacterium comprising gene sequence(s) encoding one or more transporter(s) of a branched chain amino acid operably linked to a directly or indirectly inducible promoter that is not associated with the transporter of a branched chain amino acid gene in nature.

57. The bacterium of claim 56, wherein the bacterium further comprises a genetic modification that reduces export of a branched chain amino acid from the bacterial cell.

58. The bacterium of claim 56 or claim 57, wherein the bacterium further comprises a genetic modification that reduces endogenous biosynthesis of a branched chain amino acid in the bacterium.

59. The bacterium of claim 56, wherein the bacterium further comprises gene sequence(s) encoding one or more branched chain amino acid binding protein(s).

60. The bacterium claim 56, wherein the bacterium comprises gene sequence(s) encoding one or more transporters of branched chain amino acid selected from livKHMGF and brnQ.

61. The bacterium of claim 60, wherein the livKHMGF is an E. coli livKHMGF.

62. The bacterium of claim 57, wherein the genetic modification that reduces export of a branched chain amino acid from the bacterium is gene modification in the leuE gene.

63. The bacterium of claim 62, wherein the leuE gene is deleted from the bacterium.

64. The bacterium of claim 58, wherein the genetic modification that reduces endogenous biosynthesis of a branched chain amino acid in the bacterium is a gene modification in the ilvC gene.

65. The bacterium of claim 64, wherein the ilvC gene is deleted from the bacterium.

66. The bacterium of claim 59, wherein the bacterium further comprises gene sequence(s) encoding ilvJ.

67. A bacterium comprising a genetic modification that reduces export of a branched chain amino acid from the bacterial cell.

68. The bacterium of claim 67, wherein the bacterium further comprises a genetic modification that reduces endogenous biosynthesis of a branched chain amino acid in the bacterium.

69. The bacterium of claim 67 or claim 68, wherein the bacterium further comprises gene sequence(s) encoding one or more branched chain amino acid binding protein(s).

70. The bacterium of claim 67 or claim 68, wherein the genetic modification that reduces export of a branched chain amino acid from the bacterium is gene modification in the leuE gene.

71. The bacterium of claim 70, wherein the leuE gene is deleted from the bacterium.

72. The bacterium of claim 68, wherein the genetic modification that reduces endogenous biosynthesis of a branched chain amino acid in the bacterium is a gene modification in the ilvC gene.

73. The bacterium of claim 72, wherein the ilvC gene is deleted from the bacterium.

74. The bacterium of any one of claims 69-73, wherein the bacterium further comprises gene sequence(s) encoding ilvJ.

75. A bacterium comprising a genetic modification that reduces endogenous biosynthesis of a branched chain amino acid in the bacterium.

76. The bacterium of claim 75, wherein the bacterium further comprises gene sequence(s) encoding one or more branched chain amino acid binding protein(s).

77. The bacterium of claim 75 or claim 76, wherein the genetic modification that reduces endogenous biosynthesis of a branched chain amino acid in the bacterium is a gene modification in the ilvC gene.

78. The bacterium of claim 77, wherein the ilvC gene is deleted from the bacterium.

79. The bacterium of claim 76, wherein the bacterium further comprises gene sequence(s) encoding ilvJ.

80. The bacterium of claim 1 or claim 2, wherein the bacterium is a probiotic bacterium.

81. The bacterium of claim 80, wherein the bacterium is selected from the group consisting of Bacteroides, Bifidobacterium, Clostridium, Escherichia, Lactobacillus, and Lactococcus.

82. The bacterium of claim 81, wherein the bacterium is Escherichia coli strain Nissle.

83. The bacterium of any claim 1 or claim 2, wherein the bacterium is an auxotroph in a gene that is complemented when the bacterium is present in a mammalian gut.

84. The bacterium of claim 83, wherein mammalian gut is a human gut.

85. The bacterium of claim 83 or 84, wherein the bacterium is an auxotroph in diaminopimelic acid or an enzyme in the thymidine biosynthetic pathway.

86. The bacterium of claim 1, wherein the bacterium is further engineered to harbor a gene encoding a substance toxic to the bacterium, wherein the gene is under the control of a promoter that is directly or indirectly induced by an environmental factor not naturally present in a mammalian gut.

87. The bacterium of claim 1, wherein the bacterium is a genetically engineered bacterium.

88. A pharmaceutically acceptable composition comprising the bacterium of claim 1 or claim 2; and a pharmaceutically acceptable carrier.

89. The composition of claim 88 formulated for oral administration.

90. A method of reducing the level of a branched amino acid or treating a disease associated with excess branched chain amino acid comprising the step of administering to a subject in need thereof, the composition of claim 88 or claim 89.

91. The method of claim 90, wherein the branch chain amino acid is selected from leucine, valine, and isoleucine.

92. The method of claim 91, wherein the branch chain amino acid is leucine.

93. A method of reducing the level of a branched amino acid metabolite or treating a disease associated with excess branched chain amino acid metabolite comprising the step of administering to a subject in need thereof, the composition of claim 87 or 88.

94. The method of claim 93, wherein the branch chain amino acid metabolite is selected from α-ketoisocaproate, α-keto-β-methylyvalerate, and α-ketoisovalerate.

95. The method of claim 90, wherein the disease is selected from the group consisting of: MSUD, isovaleric acidemia (IVA), propionic acidemia, methylmalonic acidemia, and diabetes ketoacidosis, as well as other diseases, for example, 3-MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria.

96. The method of claim 95, wherein the disease is MSUD.

97. A method for treating a metabolic disorder involving the abnormal catabolism of a branched amino acid in a subject, the method comprising administering the composition of claim 88 or claim 89 to the subject, thereby treating the metabolic disorder involving the abnormal catabolism of a branched chain amino acid in the subject.

98. A method for decreasing a plasma level of at least one branched chain amino acid or branched chain α keto-acid in a subject, the method comprising administering the composition of claim 88 or claim 89 to the subject, thereby decreasing the plasma level of the at least one branched chain amino acid or branched chain α keto-acid in the subject.

99. The method of claim 97 or claim 98, wherein the disease is selected from the group consisting of: MSUD, isovaleric acidemia (IVA), propionic acidemia, methylmalonic acidemia, and diabetes ketoacidosis, as well as other diseases, for example, 3-MCC Deficiency, 3-Methylglutaconyl-CoA hydratase Deficiency, HMG-CoA Lyase Deficiency, Acetyl-CoA Carboxylase Deficiency, Malonyl-CoA Decarboxylase Deficiency, short-branched chain acylCoA dehydrogenase deficiency, 2-methyl-3-hydroxybutyric acidemia, beta-ketothiolase deficiency, isobutyryl-CoA dehydrogenase deficiency, HIBCH deficiency), and 3-Hydroxyisobutyric aciduria.

100. The method of claim 90, wherein the subject has a disease caused by activation of mTor.

101. The method of claim 100, wherein the disease caused by activation of mTor is cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, or Friedrich's ataxia.
